(19) United States
(12) Patent Application Publication
(10) Pub. No.: US 2020/0299661 A1
(43) Pub. Date: Sep. 24, 2020

(54) CPF1-RELATED METHODS AND COMPOSITIONS FOR GENE EDITING

(71) Applicant: EDITAS MEDICINE, INC., Cambridge, MA (US)

(72) Inventors: Jennifer Gori, Jamaica Plain, MA (US); John Zuris, Cambridge, MA (US); Hariharan Jayaram, Cambridge, MA (US)

(73) Assignee: EDITAS MEDICINE, INC., Cambridge, MA (US)

(21) Appl. No.: 16/899,302

(22) Filed: Jun. 11, 2020

Related U.S. Application Data
(63) Continuation of application No. PCT/US18/65032, filed on Dec. 11, 2018.
(60) Provisional application No. 62/597,118, filed on Dec. 11, 2017.
(60) Provisional application No. 62/623,501, filed on Jan. 29, 2018.
(60) Provisional application No. 62/664,905, filed on Apr. 30, 2018.
(60) Provisional application No. 62/746,494, filed on Oct. 16, 2018.

(51) Int. Cl. C12N 9/22; C12N 5/0783; C12N 15/11; C12N 5/0789; A61K 35/17; A61K 35/28; C12Q 1/44

(52) U.S. Cl. CPC C12N 9/22; C12N 5/0636; C12N 15/11; C12N 5/0647; A61K 35/17; A61K 2035/124; C12Q 1/44; C12N 2510/00; C12N 2310/20; C12N 2800/80; A61K 35/28

(57) ABSTRACT

The present disclosure relates to CRISPR/Cpf1-related methods and components for editing a target nucleic acid sequence and/or modulating expression of a target nucleic acid sequence, as well as methods and compositions for evaluating such editing and/or modulation of expression.

### CROSS-REFERENCE TO RELATED APPLICATIONS

This application is a continuation of International Patent Application No. PCT/US2018/065032, filed Dec. 11, 2018, which claims priority to U.S. Provisional Application Ser. No. 62/597,118, filed Dec. 11, 2017, U.S. Provisional Application Ser. No. 62/623,501, filed Jan. 29, 2018, U.S. Provisional Application Ser. No. 62/664,905, filed Apr. 30, 2018, and U.S. Provisional Application Ser. No. 62/746,494, filed Oct. 16, 2018, to each of which priority is claimed and the contents of each of which are incorporated herein in their entireties.

### SEQUENCE LISTING

The specification further incorporates by reference the Sequence Listing submitted herewith via EFS on Jun. 11, 2020. Pursuant to 37 C.F.R. § 1.52(e)(5), the Sequence Listing text file, identified as 084177_0231_SL.txt, is 444,079 bytes and was created on Jun. 11, 2020. The entire contents of the Sequence Listing are hereby incorporated by reference. The Sequence Listing does not extend beyond the scope of the specification and thus does not contain new matter.

### FIELD

The present disclosure relates to CRISPR/Cpf1-related methods and components for editing a target nucleic acid sequence and/or modulating expression of a target nucleic acid sequence, as well as methods and compositions for evaluating such editing and/or modulation of expression.

### BACKGROUND

CRISPRs (Clustered Regularly Interspaced Short Palindromic Repeats) evolved in bacteria and archaea as an adaptive immune system to defend against viral attack. Upon exposure to a virus, short segments of viral DNA are integrated into the CRISPR locus. RNA is transcribed from a portion of the CRISPR locus that includes the viral sequence. That RNA, which contains sequence complementary to the viral genome, mediates targeting of a Cpf1 protein to a target sequence in the viral genome. The Cpf1 protein (“CRISPR from Prevotella and Franciscella 1”), also known as Cas12a, in turn, cleaves and thereby silences the viral target.

Recently, the CRISPR/Cpf1 system has been adapted for genome editing in eukaryotic cells. The introduction of site-specific double strand breaks (DSBs) allows for target sequence alteration through endogenous DNA repair mechanisms, for example non-homologous end-joining (NHEJ) or homology-directed repair (HDR).

### SUMMARY

The instant disclosure provides improved CRISPR/Cpf1-related methods and components for the editing of a target nucleic acid sequence and/or modulating the expression of a target nucleic acid sequence, e.g., in therapeutically-relevant cell lines and with respect to therapeutically-relevant target sequences, as well as strategies for evaluating the efficiency of such target editing and/or modulation of expression.

In one aspect, the present disclosure relates to the use of CRISPR/Cpf1-mediated editing of therapeutically-relevant target sites in therapeutically-relevant cell populations. For example, but not by way of limitation, the present disclosure provides isolated cells that include a modification of a therapeutically-relevant target site. In certain embodiments, the cell is a T cell, e.g., CD8⁺ T cell, a CD8⁺ naïve T cell, a CD4⁺ central memory T cell, a CD8⁺ central memory T cell, a CD4⁺ effector memory T cell, a CD4⁺ effector memory T cell, a CD4⁺ T cell, a CD4⁺ stem cell memory T cell, a CD8⁺ stem cell memory T cell, a CD4⁺ helper T cell, a regulatory T cell, a cytotoxic T cell, a natural killer T cell, a CD4+ naïve T cell, a TH17 CD4⁺ T cell, a TH1 CD4⁺ T cell, a TH2 CD4⁺ T cell, a TH9 CD4⁺ T cell, a CD4⁺ Foxp3⁺ T cell, a CD4⁺ CD25⁺ CD127⁻ T cell or a CD4⁺ CD25⁺ CD127⁻ Foxp3⁺ T cell. In certain embodiments, the cell is a lymphoid progenitor cell, a hematopoietic stem cell (HSC), a human umbilical cord blood-derived erythroid progenitor (HUDEP) cell, a natural killer cell or a dendritic cell. In certain embodiments, the cell is a HSC or a HUDEP cell.

In certain embodiments, the present disclosure provides an isolated cell or a population of cells that include a modification, e.g., disruption, in an HBG locus, e.g., generated by the delivery of an RNP complex comprising a Cpf1 RNA-guided nuclease and a gRNA molecule that targets the HBG locus including, for example, the regulatory region of an HBG gene. In certain embodiments, an RNP complex includes a complex between a Cpf1 RNA-guided nuclease and a gRNA molecule. In certain embodiments, any region of the HBG locus can be targeted. In certain embodiments, a cis-regulatory region of the HBG gene is targeted. In certain embodiments, the instant disclosure relates to the use of CRISPR/Cpf1-mediated editing, e.g., disruption, of the promoter region of the HBG locus. In certain embodiments, the instant disclosure relates to the use of CRISPR/Cpf1-mediated editing of the −800 to the −60 nt promoter region of the HBG locus, e.g., the −110 nt promoter region. In certain embodiments, the cis-regulatory region of the HBG locus can be edited, e.g., disrupted. For example, but not by way of limitation, CRISPR/Cpf1-mediated editing can be employed to disrupt the CAAT box present in the cis-regulatory region of the HBG locus. Disruption of the HBG promoter region generally and the CAAT box specifically can be accomplished via the delivery of a CRISPR/Cpf1 editing system targeted to those sequences. Non-limiting examples of gRNA molecules for use in such a CRISPR/Cpf1 editing system targeting those sequences of the HBG locus are identified in FIGS. 6, 9 and 11 and Table 19. In certain embodiments, a gRNA molecule that targets the HBG gene sequence comprises the sequence of gRNA molecule, referred to as HBG1-1.

In certain embodiments, the instant disclosure is directed to an isolated CRISPR/Cpf1-edited cell wherein the −110 nt promoter region of the HBG locus is disrupted using a complex comprising a CRISPR/Cpf1 RNA guided nuclease and a guide RNA which targets the −110 nt promoter region of the HBG locus. In certain embodiments, such a CRISPR/Cpf1-edited cell can include one or more components of a CRISPR/Cpf1 editing system. In certain embodiments, such a CRISPR/Cpf1-edited cell does not include one or more components of a CRISPR/Cpf1 editing system, as determined using suitable methods used to detect such components. In certain embodiments, the instant disclosure is directed to a population of CRISPR/Cpf1-edited cells wherein the −110 nt promoter region of the HBG locus is disrupted using a complex comprising a CRISPR/Cpf1 RNA guided nuclease and a guide RNA which targets the −110 nt promoter region of the HBG locus. In certain embodiments, such a CRISPR/Cpf1-edited cell population can include cells comprising one or more components of a CRISPR/Cpf1 editing system. In certain embodiments, such a CRISPR/Cpf1 edited cell population does not include one or more components of a CRISPR/Cpf1 editing system, as determined using suitable methods used to detect such components. In certain embodiments, the instant disclosure is directed to a CRISPR/Cpf1-edited cell wherein the CAAT box present in the HBG promoter region is disrupted using a complex comprising a CRISPR/Cpf1 RNA guided nuclease and a guide RNA which targets the CAAT box present in the promoter region of the HBG locus. In certain embodiments, such a cell comprises one or more components of a CRISPR/Cpf1 editing system. In certain embodiments, such a CRISPR/Cpf1-edited cell does not include one or more components of a CRISPR/Cpf1 editing system, as determined using suitable methods used to detect such components. In certain embodiments, the instant disclosure is directed to a population of CRISPR/Cpf1-edited cells wherein the CAAT box present in the HBG promoter region is disrupted using a complex comprising a CRISPR/Cpf1 RNA guided nuclease and a guide RNA which targets the CAAT box present in the promoter region of the HBG locus. In certain embodiments, such a CRISPR/Cpf1-edited cell population can include cells comprising one or more components of a CRISPR/Cpf1 editing system.

In certain embodiments, the present disclosure provides a CRISPR/Cpf1-edited cell or a population of cells edited using CRISPR/Cpf1 that include a modification, e.g., disruption, in the erythroid cell specific expression of a transcriptional repressor, BCL11a, e.g., generated by the delivery of a complex comprising a Cpf1 RNA-guided nuclease and a gRNA molecule that targets the BCL11a gene sequence. In certain embodiments, any region of the BCL11a gene sequence can be targeted. For example, but not by way of limitation, the erythroid enhancer region of the BCL11a gene can be targeted, e.g., the erythroid enhancer region between +55 kb and +62 kb from the Transcription Start Site (TSS). In certain embodiments, CRISPR/Cpf1-mediated editing can be employed to disrupt the GATA1 binding motif of BCL11a, present in the +58 DHS region of intron 2 of the BCL11a gene. Disruption of the GATA1 binding motif of BCL11a can be accomplished via the delivery of a CRISPR/Cpf1 editing system targeted to that motif. Non-limiting examples of gRNA molecules for use in such a CRISPR/Cpf1 editing system targeting the GATA1 motif of BCL11a are identified in FIGS. 7, 10 and 12.

In certain embodiments, the instant disclosure is directed to a CRISPR/Cpf1-edited cell wherein the +58 DHS region of intron 2 of the BCL11a gene is disrupted. In certain embodiments, such a CRISPR/Cpf1-edited cell can include one or more components of a CRISPR/Cpf1 editing system. In certain embodiments, the instant disclosure is directed to a population of CRISPR/Cpf1-edited cells wherein the +58 DHS region of intron 2 of the BCL11a gene is disrupted. In certain embodiments, such a CRISPR/Cpf1-edited cell population can include cells comprising one or more components of a CRISPR/Cpf1 editing system. In certain embodiments, the instant disclosure is directed to a CRISPR/Cpf1-edited cell wherein the GATA1 motif of the BCL11a gene is disrupted. In certain embodiments, such a CRISPR/Cpf1-edited cell can include one or more components of a CRISPR/Cpf1 editing system. In certain embodiments, the instant disclosure is directed to a population of CRISPR/Cpf1-edited cells wherein the GATA1 motif of the BCL11a gene is disrupted. In certain embodiments, such a CRISPR/Cpf1-edited cell population can include cells comprising one or more components of a CRISPR/Cpf1 editing system. In certain embodiments, one or more components of a CRISPR/Cpf1 system used to modify or disrupt the BCL11a gene in a cell or population of cells are undetectable using suitable means used to detect such components.

In certain embodiments, the present disclosure provides an isolated CRISPR/Cpf1-edited T cell or population of CRISPR/Cpf1-edited T cells that include a modification, e.g., disruption, in one or more endogenous genes of a T cell. In certain embodiments, the instant disclosure relates to the use of CRISPR/Cpf1-mediated editing of an endogenous gene of a T cell selected from the group consisting of FAS, BID, CTLA4, PDCD1, CBLB, PTPN6, B2M, TRAC, CIITA, TRBC and any combination thereof. For example, but not by way of limitation, the modification is generated by the delivery of one or more complexes comprising a Cpf1 RNA-guided nuclease and a gRNA molecule, e.g., RNP complexes, that targets a portion of a FAS gene sequence, a portion of a BID gene sequence, a portion of a CTLA4 gene sequence, a portion of a PDCD1 gene sequence, a portion of a CBLB gene sequence, a portion of a PTPN6 gene sequence, a portion of a B2M gene sequence, a portion of a TRAC gene sequence, a portion of a CIITA gene sequence, a portion of a TRBC gene sequence or a combination thereof. For example, and not by way of limitation, two or more, three or more, four or more, five or more, six or more, seven or more, eight or more, nine or more or ten complexes, e.g., RNP complexes, can be delivered, where each of the complexes target a different gene. In certain embodiments, the gRNA can be complementary to either strand of the gene to be targeted. In certain embodiments, the gRNA molecule can target a regulatory region, an intron or an exon of the gene to be targeted.

In certain embodiments, the CRISPR/Cpf1 system encompassed by the disclosure herein targets the TRAC gene, e.g., to generate an isolated CRISPR/Cpf1-edited T cell or population of CRISPR/Cpf1-edited T cells that include a modification, e.g., disruption, in the TRAC gene. In certain embodiments, the CRISPR system comprises a gRNA complementary to a portion of the TRAC gene sequence. In certain embodiments, the gRNA can be complementary to either strand of the TRAC gene. In certain embodiments, the targeted portion of the TRAC gene sequence is within the coding sequence of the TRAC gene. In certain embodiments, the targeted portion of the TRAC gene sequence is within an exon. In certain embodiments, the targeted portion of the TRAC gene sequence is within an intron. In certain embodiments, the targeted portion of the TRAC gene sequence is within a regulatory region of the gene. In certain embodiments, more than one sequence is targeted and the targeted portions of the TRAC gene sequence are within one or more exons, one or more introns, one or more regulatory regions or one or more exons, one or more introns and one or more regulatory regions. In certain embodiments, a targeting domain of a gRNA molecule for use in such a CRISPR/Cpf1 system targeting TRAC comprises a targeting domain sequence listed in Tables 2 and 3.

In certain embodiments, the CRISPR/Cpf1 system encompassed by the disclosure herein targets the TRBC gene, e.g., to generate an isolated CRISPR/Cpf1-edited T cell or population of CRISPR/Cpf1-edited T cells that include a modification, e.g., disruption, in the TRBC gene. In certain embodiments, the CRISPR system comprises a gRNA complementary to a portion of the TRBC gene sequence. In certain embodiments, the gRNA can be complementary to either strand of the TRBC gene. In certain embodiments, the targeted portion of the TRBC gene sequence is within the coding sequence of the TRBC gene. In certain embodiments, the targeted portion of the TRBC gene sequence is within an exon. In certain embodiments, the targeted portion of the TRBC gene sequence is within an intron. In certain embodiments, the targeted portion of the TRBC gene sequence is within a regulatory region of the gene. In certain embodiments, more than one sequence is targeted and the targeted portions of the TRBC gene sequence are within one or more exons, one or more introns, one or more regulatory regions or one or more exons, one or more introns and one or more regulatory regions. In certain embodiments, a targeting domain of a gRNA molecule for use in such a CRISPR/Cpf1 system targeting TRBC comprises a targeting domain sequence listed in Tables 4 and 5.

In certain embodiments, the CRISPR/Cpf1 system encompassed by the disclosure herein targets the B2M gene, e.g., to generate an isolated CRISPR/Cpf1-edited T cell or population of CRISPR/Cpf1-edited T cells that include a modification, e.g., disruption, in the B2M gene. In certain embodiments, the CRISPR system comprises a gRNA complementary to a portion of the B2M gene sequence. In certain embodiments, the gRNA can be complementary to either strand of the B2M gene. In certain embodiments, the targeted portion of the B2M gene sequence is within the coding sequence of the B2M gene. In certain embodiments, the targeted portion of the B2M gene sequence is within an exon. In certain embodiments, the targeted portion of the B2M gene sequence is within an intron. In certain embodiments, the targeted portion of the B2M gene sequence is within a regulatory region of the gene. In certain embodiments, more than one sequence is targeted and the targeted portions of the B2M gene sequence are within one or more exons, one or more introns, one or more regulatory regions or one or more exons, one or more introns and one or more regulatory regions. In certain embodiments, a targeting domain of a gRNA molecule for use in such a CRISPR/Cpf1 system targeting B2M comprises a targeting domain sequence listed in Tables 6, 7 and 8. In certain embodiments, a targeting domain of a gRNA molecule for use in such a CRISPR/Cpf1 system targeting B2M comprises the nucleic acid sequence AGUGGGGGUGAAUUCAGUGU.

In certain embodiments, the CRISPR/Cpf1 system encompassed by the disclosure herein targets the CIITA gene, e.g., to generate an isolated CRISPR/Cpf1-edited T cell or population of CRISPR/Cpf1-edited T cells that include a modification, e.g., disruption, in the CIITA gene. In certain embodiments, the CRISPR system comprises a gRNA complementary to a portion of the CIITA gene sequence. In certain embodiments, the gRNA can be complementary to either strand of the CIITA gene. In certain embodiments, the targeted portion of the CIITA gene sequence is within the coding sequence of the CIITA gene. In certain embodiments, the targeted portion of the CIITA gene sequence is within an exon. In certain embodiments, the targeted portion of the CIITA gene sequence is within an intron. In certain embodiments, the targeted portion of the CIITA gene sequence is within a regulatory region of the gene. In certain embodiments, more than one sequence is targeted and the targeted portions of the CIITA gene sequence are within one or more exons, one or more introns, one or more regulatory regions or one or more exons, one or more introns and one or more regulatory regions. In certain embodiments, a targeting domain of a gRNA molecule for use in such a CRISPR/Cpf1 system targeting CIITA comprises a targeting domain sequence listed in Table 9.

In certain embodiments, the CRISPR/Cpf1 system encompassed by the disclosure herein targets a combination of two or more of the TRAC, CIITA, TRBC and B2M genes, using a gRNA which targets one or more exons, one or more introns or one or more regulatory regions of two or more of these genes, e.g., to generate an isolated CRISPR/Cpf1-edited T cell or population of CRISPR/Cpf1-edited T cells that include a modification, e.g., disruption, in two or more of the TRAC, CIITA, TRBC and B2M genes. In certain embodiments, a CRISPR/Cpf1 system of the present disclosure can include one or more complexes comprising a Cpf1 RNA-guided nuclease and a gRNA molecule that target one or more of genes, e.g., selected from the group consisting of B2M, TRAC, CIITA and TRBC. For example, but not by way of limitation, a CRISPR/Cpf1 system of the present disclosure can include (a) a first RNP complex comprising a first gRNA that includes a first targeting domain that is complementary to a target sequence of a first gene and a first Cpf1 RNA-guided nuclease; and (b) a second RNP complex comprising a second gRNA molecule that includes a second targeting domain that is complementary to a target sequence of a second gene and a second Cpf1 RNA-guided nuclease. In certain embodiments, the first gene and the second gene are selected from the group consisting of B2M, TRAC, CIITA and TRBC. The CRISPR/Cpf1 system can further include additional RNP complexes targeting one or more additional genes. For example, but not by way of limitation, in the case of multiplexing, each RNP complex can contain the same Cpf1 protein or each RNP complex can include different Cpf1 proteins, e.g., Cpf1 protein variants.

In certain embodiments, an isolated cell, e.g., isolated CRISPR/Cpf1-edited HSCs or CRISPR/Cpf1-edited T cells, or population of such CRISPR/Cpf1-edited cells do not include one or more components of a CRISPR/Cpf1 editing system. In certain embodiments, less than about 10%, less than about 5% or less than about 1% of the CRISPR/Cpf1-edited cells in the population of cells include one or more components of a CRISPR/Cpf1 editing system, as determined using suitable means to detect such components. In certain embodiments, at least about 5%, at least about 10%, at least about 20%, at least about 30%, at least about 40%, at least about 50%, at least about 60%, at least about 70%, at least about 80% or at least about 90% of the cells in the population of cells are edited and/or modified, e.g., have a disruption in the BCL11a gene, disruption in an HBG locus and/or a disruption in one or more genes selected from FAS, BID, CTLA4, PDCD1, CBLB, PTPN6, B2M, TRAC, CIITA and TRBC. In certain embodiments, the population of cells has greater than about 15% editing, greater than about 20% editing, greater than about 25% editing, greater than about 30% editing, greater than about 35% editing, greater than about 40% editing, greater than about 45% editing, greater than about 50% editing, greater than about 55% editing or greater than about 60% editing. In certain embodiments, at least about 5%, at least about 10%, at least about 20%, at least about 30%, at least about 40%, at least about 50%, at least about 60%, at least about 70%, at least about 80% or at least about 90% of the cells in the population of cells have a productive indel.

In another aspect, the present disclosure relates to modified Cpf1 proteins and their use in CRISPR/Cpf1-related methods for editing a target nucleic acid sequence and/or modulating expression of a target nucleic acid sequence. The present disclosure further provides nucleic acids that encode the modified Cpf1 proteins.

In certain embodiments, the modified Cpf1 proteins are derived from a Cpf1 protein selected from the group consisting of Acidaminococcus sp. strain BV3L6 Cpf1 protein (AsCpf1), Francisella novicida U112 (FnCpf1), Moraxella bovoculi 237 (MbCpf1), Candidatus Methanomethylphilus alvus Mx1201 (CMaCpf1), Sneatia amnii (SaCpfq), Moraxella lacunata (M1Cpf1), Moraxella bovoculi AAX08_00205 (Mb2Cpf1), Moraxella bovoculi AAX11_00205 (Mb3 Cpf1), Lachnospiraceae bacterium ND2006 Cpf1 protein (LbCpf1), Lachnospiraceae bacterium MA2020 (Lb5Cpf1), Lachnospiraceae bacterium MC2017 (Lb4Cpf1), Flavobacterium brachiophilum FL-15 (FbCpf1), Thiomicrospira sp. XS5 (TsCpf1), Parcubacteria group bacterium GW2011 (PgCpf1), Candidatus Roizmanbacteria bacterium GW2011 (CRbCpf1), Candidatus Peregrinbacteria bacterium GW2011 (CPbCpf1), Bytrivibrio sp. NC3005 (BsCpf1), Butyrivibrio fibrisolvens (BfCpf1), Prevotella bryantii B14 (Pb2Cpf1) and Bacteroidetes oral taxon 274 (BoCpf1) (see, e.g., Zetsche et al., bioRxiv 134015; doi: https://doi.org/10.1101/134015, the contents of which is incorporated by reference herein in its entirety).

In certain embodiments, the modified Cpf1 protein comprises a nuclear localization signal (NLS). For example, but not by way of limitation, such NLS sequences are selected from the group consisting of: the nucleoplasmin NLS (nNLS) (SEQ ID NO: 1) and the simian virus 40 “SV40” NLS (sNLS) (SEQ ID NO: 2).

In certain embodiments, the NLS sequence of the modified Cpf1 protein is positioned at or near the C-terminus of the Cpf1 protein sequence. For example, but not by way of limitation, the modified Cpf1 protein can be selected from the following: His-AsCpf1-nNLS (SEQ ID NO: 3); His-AsCpf1-sNstaneyLS (SEQ ID NO: 4); and His-AsCpf1-sNLS-sNLS (SEQ ID NO: 5). In certain embodiments, the NLS sequence of the modified Cpf1 protein is positioned at or near the N-terminus of the Cpf1 protein sequence. For example, but not by way of limitation, the modified Cpf1 protein can be selected from the following: His-sNLS-AsCpf1 (SEQ ID NO: 6), His-sNLS-sNLS-AsCpf1 (SEQ ID NO: 7), and sNLS-sNLS-AsCpf1 (SEQ ID NO: 8). In certain embodiments, the modified Cpf1 protein comprises NLS sequences positioned at or near both the N-terminus and C-terminus of the Cpf1 protein sequence. For example, but not by way of limitation, the modified Cpf1 protein can be selected from the following: His-sNLS-AsCpf1-sNLS (SEQ ID NO: 9) and His-sNLS-sNLS-AsCpf1-sNLS-sNLS (SEQ ID NO: 10). Additional permutations of the identity and N-terminal/C-terminal positions of NLS sequences, e.g., appending two or more nNLS sequences or combinations of nNLS and sNLS sequences (or other NLS sequences), as well as sequences with and without purification sequences, e.g., six-histidine sequences, are within the scope of the instantly disclosed subject matter.

In certain embodiments, the modified Cpf1 protein comprises an alteration (e.g., a deletion or substitution) at one or more cysteine residues of the Cpf1 protein sequence. For example, but not by way of limitation, modified Cpf1 protein comprises an alteration at a position selected from the group consisting of: C65, C205, C334, C379, C608, C674, C1025 and C1248. In certain embodiments, the modified Cpf1 protein comprises a substitution of one or more cysteine residues for a serine or alanine. In certain embodiments, the modified Cpf1 protein comprises an alteration selected from the group consisting of: C65S, C205S, C334S, C379S, C608S, C674S, C1025S and C1248S. In certain embodiments, the modified Cpf1 protein comprises an alteration selected from the group consisting of: C65A, C205A, C334A, C379A, C608A, C674A, C1025A and C1248A. In certain embodiments, the modified Cpf1 protein comprises alterations at positions C334 and C674 or C334, C379 and C674. In certain embodiments, the modified Cpf1 protein comprises the following alterations: C334S and C674S or C334S, C379S and C674S. In certain embodiments, the modified Cpf1 protein comprises the following alterations: C334A and C674A or C334A, C379A and C674A. In certain embodiments, the modified Cpf1 protein comprises both one or more cysteine residue alterations as well as the introduction of one or more NLS sequences, e.g., His-AsCpf1-nNLS Cys-less (SEQ ID NO: 11) or His-AsCpf1-nNLS Cys-low (SEQ ID NO: 12). In certain embodiments, the Cpf1 protein comprising a deletion or substitution in one or more cysteine residues exhibits reduced aggregation.

In a further aspect, the present disclosure provides methods of modifying one or more target sequences in a cell. In certain embodiments, such methods include contacting a cell or a population of cells with (a) a gRNA molecule complementary to the target sequence of interest; and (b) a Cpf1 RNA-guided nuclease. In certain embodiments, the Cpf1 RNA-guided nuclease modifies the target sequence of interest in the cell or population of cells. In certain embodiments, the cell is a T cell, a hematopoietic stem cell (HSC) or a human umbilical cord blood-derived erythroid progenitor (HUDEP) cell. In certain embodiments, at least about 10%, at least about 20%, at least about 30%, at least about 40%, at least about 50%, at least about 60%, at least about 70%, at least about 80% or at least about 90% of the cells in the population of cells are modified. In certain embodiments, the target sequence of interest is an HBG1 gene sequence, e.g., promoter region, and the gRNA molecule includes the sequence of gRNA molecule HBG1-1. In certain embodiments, the target sequence of interest is an BCL11a gene sequence. Alternatively, the target nucleic acid sequence is selected from the group consisting of: a portion of a FAS gene sequence, a portion of a BID gene sequence, a portion of a CTLA4 gene sequence, a portion of a PDCD1 gene sequence, a portion of a CBLB gene sequence, a portion of a PTPN6 gene sequence, a portion of a B2M gene sequence, a portion of a TRAC gene sequence, a portion of the CIITA gene sequence, a portion of a TRBC gene sequence and a combination thereof.

The present disclosure further provides methods for modifying one or more, e.g., two or more, three or more or four or more, genes in a cell that include contacting the cell with (a) a first RNP complex comprising a first gRNA that includes a first targeting domain that is complementary to a target sequence of a first gene and a first Cpf1 RNA-guided nuclease; and (b) a second RNP complex comprising a second gRNA molecule that includes a second targeting domain that is complementary to a target sequence of a second gene and a second Cpf1 RNA-guided nuclease. In certain embodiments, the methods can further include (c) a third RNP complex comprising a third gRNA molecule comprising a third targeting domain that is complementary to a target sequence of a third gene and a third Cpf1 RNA-guided nuclease and/or (d) a fourth RNP complex comprising a fourth gRNA molecule comprising a fourth targeting domain that is complementary to a target sequence of a fourth gene and a fourth Cpf1 RNA-guided nuclease. In certain embodiments, each RNP complex can comprise the same Cpf1 protein or each RNP complex can include different Cpf1 proteins, e.g., Cpf1 protein variants. In certain embodiments, the methods for modifying one or more, e.g., two or more, three or more or four or more genes in a cell can include contacting the cell with (a) a first gRNA that includes a first targeting domain that is complementary to a target sequence of a first gene; (b) a second gRNA molecule that includes a second targeting domain that is complementary to a target sequence of a second gene; and (c) a Cpf1 RNA-guided nuclease disclosed herein or encoded by a nucleic acid encoding a disclosed Cpf1 RNA-guided nuclease. In certain embodiments, the methods can further include (d) a third gRNA molecule comprising a third targeting domain that is complementary to a target sequence of a third gene and/or (e) a fourth gRNA molecule comprising a fourth targeting domain that is complementary to a target sequence of a fourth gene, wherein the Cpf1 RNA-guided nuclease modifies the first gene, the second gene, the third gene and/or the fourth gene. In certain embodiments, the first gene, the second gene, the third gene and the fourth gene are selected from the group consisting of the B2M, TRAC, CIITA and TRBC genes. In certain embodiments, the cell is a T cell.

In another aspect, the present disclosure relates to methods of treating a subject by administering to the subject, one or more cells modified using the CRISPR/Cpf1 systems encompassed by the present disclosure. In certain embodiments, the one or more cells are modified ex vivo or in vitro and then administered to the subject. In certain embodiments, the methods for treating a subject includes contacting a cell obtained from the subject with a CRISPR/Cpf1 system comprising: (a) a gRNA molecule complementary to a target sequence of a target nucleic acid; and (b) a Cpf1 RNA-guided nuclease disclosed herein. In certain embodiments, the present disclosure relates to a method of treating a subject in need thereof by administering to the subject one or more cells that are obtained from a donor and genetically modified ex vivo or in vitro using a CRISPR/Cpf1 system of the present disclosure prior to administration to the subject. In certain embodiments, the subject suffers from a hemoglobinopathy, e.g., sickle cell disease or beta-thalassemia. In certain embodiments, the subject suffers from cancer or an autoimmune disorder.

In certain embodiments, the present disclosure further provides methods of administering a population of cells to a subject suffering from a hemoglobinopathy, where the population of cells include a modification in an HBG gene sequence or a BCL11a gene sequence generated by the delivery of a complex comprising a Cpf1 RNA-guided nuclease and a gRNA molecule that targets the HBG gene sequence or the BCL11a gene sequence. In certain embodiments, at least about 5%, at least about 10%, at least about 20%, at least about 30%, at least about 40%, at least about 50%, at least about 60%, at least about 70%, at least about 80% or at least about 90% of the cells in the population of cells are modified. In certain embodiments, the cell is a hematopoietic stem cell (HSC) or a human umbilical cord blood-derived erythroid progenitor (HUDEP) cell.

In a further aspect, the present disclosure provides gRNA molecules for the targeting of a nucleic acid sequence of interest to generate modified cells, e.g., CRISPR/Cpf1-edited cells. In certain embodiments, the gRNA molecule includes a first targeting domain that is complementary to a target sequence, wherein the target sequence is a HBG gene sequence or a BCL11a gene sequence. Non-limiting examples of such gRNAs are provided in FIGS. 6-12 and 46 and Table 19. In certain embodiments, the present disclosure provides a CRISPR/Cpf1 system that includes a gRNA molecule that when introduced into a cell, an indel is formed at or near the target sequence complementary to the first targeting domain of the gRNA molecule and/or when a CRISPR/Cpf1 system comprising the gRNA molecule is introduced into a cell, a deletion is created in a sequence complementary to the gRNA first targeting domain in the HBG1 or HBG2 promoter region. In certain embodiments, a CRISPR/Cpf1 system that includes a gRNA molecule of the present disclosure results in an increase in the expression of fetal hemoglobin when introduced into a cell. In certain embodiments, a CRISPR/Cpf1 system that includes a gRNA molecule of the present disclosure results in an increase in the expression of fetal hemoglobin in an amount suitable to partially or completely alleviate the symptoms of a hemoglobinopathy, e.g., sickle cell disease or beta-thalassemia. For example, but not by way of limitation, expression of fetal hemoglobin can increased by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to the level of expression of fetal hemoglobin in a cell or population of cells without a disruption in the BCL11a gene or an HBG locus and/or gene. In certain embodiments, the increase in the expression of fetal hemoglobin can be greater than about 1 picogram (pg), greater than about 2 pg, greater than about 3 pg, greater than about 4 pg, greater than about 5 pg, greater than about 6 pg, greater than about 7 pg, greater than about 8 pg, greater than about 9 pg or greater than about 10 pg.

The present disclosure further provides gRNA molecules that include a first targeting domain that is complementary to a target sequence, wherein the target sequence is selected from the group consisting of a portion of a B2M gene sequence, a portion of a TRAC gene sequence, a portion of a CIITA gene sequence, a portion of a TRBC gene sequence and a combination thereof. Non-limiting examples of such gRNAs are provided in Tables 2-9.

The present disclosure provides compositions that include the gRNA molecules disclosed herein. In certain embodiments, the gRNA molecules comprise the gRNAs disclosed in Tables 2-9 and 19 and FIGS. 6-12. In certain embodiments, the gRNAs target the chromosomal locations (e.g., genomic coordinates) provided in Table 18. In certain embodiments, the compositions can further include a Cpf1 protein, e.g., to produce an RNP complex. In the certain embodiments, the present disclosure provides a composition that comprises one or more RNP complexes, e.g., a population of RNP complexes, where each RNP complex targets a different gene or region of a gene. In certain embodiments, the compositions can be used to treat a subject in need thereof, e.g., a subject suffering from cancer, an autoimmune disorder or a hemoglobinopathy.

In another aspect, the present disclosure relates to genome-editing systems for modifying a target nucleic acid sequence. In certain embodiments, the genome editing system can include a gRNA molecule; and a Cpf1 RNA-guided nuclease disclosed herein. The present disclosure further provides a multiplex genome editing system, e.g., for the editing of two or more genes selected from the group consisting of B2M, TRAC, CIITA and TRBC.

In a further aspect, the present disclosure relates to methods for evaluating the CRISPR/Cpf1-mediated editing of a target nucleic acid sequence and/or modulation of expression of a target nucleic acid sequence, as well as components for accomplishing the same.

In certain embodiments, the methods for evaluating CRISPR/Cpf1-mediated editing of a target nucleic acid sequence and/or modulation of expression of a target nucleic acid sequence comprise comparing the activity of a test Cpf1 protein to a control Cpf1 protein with respect to a target nucleic acid sequence. In certain embodiments, the test Cpf1 protein comprises one or more modifications relative to the control, e.g., wild type, Cpf1 protein. Examples of such modifications include, but are not limited to, the incorporation of one or more NLS sequence, the incorporation of a six-histidine purification sequence, and the alteration of a Cpf1 protein cysteine amino acid, as well as combinations thereof.

In certain embodiments, the methods for evaluating CRISPR/Cpf1-mediated editing of a target nucleic acid sequence and/or modulation of expression of a target nucleic acid sequence comprises comparing the activity with respect to a “matched site” target nucleic acid sequence of a test Cpf1 protein to a control Cas9 protein. As used herein, a matched site target nucleic acid sequence incorporates both the requirements to be edited by Cpf1 as well as Cas9, e.g., the TTTV AsCpf1 wild type protospacer adjacent motif (“PAM”) and a NGG SpCas9 wild type PAM. As noted above, the test Cpf1 protein can comprise one or more modifications relative to the wild type Cpf1 protein. Examples of such modifications include, but are not limited to, the aforementioned modifications to incorporate one or more NLS sequence, to incorporate a six-histidine purification sequence, and the alteration of a Cpf1 protein cysteine amino acid, as well as combinations thereof.

In certain embodiments, the present disclosure relates to assays for the comparison of CRISPR/Cpf1-mediated editing of a target nucleic acid sequence and/or modulation of expression of a target nucleic acid sequence by a test CRISPR/Cpf1 genome editing system to a control RNA-guided nuclease genome editing system. For example, but not by way of limitation, the test and control genome editing systems can differ by any one or more of the following aspects: the sequence of the RNA-guided nuclease; the source, e.g., method of manufacture, of a component of a genome editing system; the formulation of one or more component of the genome editing system; and the identity of the cell into which the genome editing system is introduced, e.g., cell type or method of preparation of the cell. In certain embodiments, the assays described herein allow for quality control analysis of test genome editing systems. In certain embodiments, the assays of the present disclosure will assess CRISPR/Cpf1-mediated editing of a target nucleic acid sequence and/or modulation of expression of a target nucleic acid sequence wherein the target comprises a matched site sequence.

In certain embodiments, the use of a matched site target nucleic acid allows for the assay and/or evaluation of CRISPR/Cpf1-mediated versus CRISPR/Cas9-mediated editing (or editing by another CRISPR-based system) of a target nucleic acid sequence and/or modulation of expression of a target nucleic acid sequence.

In certain embodiments, the use of a matched site target nucleic acid allows for the assay and/or evaluation of CRISPR/Cpf1-mediated versus CRISPR/Cas9-mediated editing (or editing by another CRISPR-based system) of a target nucleic acid sequence and/or modulation of expression of a target nucleic acid sequence in specific cell types. For example, but not by way of limitation, such methods can be used to evaluate CRISPR/Cpf1-mediated versus CRISPR/Cas9-mediated editing of a target nucleic acid sequence and/or modulation of expression of a target nucleic acid sequence in T cells, hematopoietic stem cells (HSCs, including, but not limited to, CD34⁺ HSCs), and human umbilical cord blood-derived erythroid progenitor cells (HUDEPs), among other cell types.

In certain embodiments, the use of a matched site target nucleic acid allows for the assay and/or evaluation of a CRISPR/Cpf1-mediated versus CRISPR/Cas9-mediated editing (or editing by another CRISPR-based system) of a target nucleic acid sequence and/or modulation of expression of a target nucleic acid sequence with respect to particular attributes of the CRISPR/Cpf1-mediated editing system employed. For example, but not by way of limitation, such methods can be used to evaluate CRISPR/Cpf1-mediated versus CRISPR/Cas9-mediated editing of a target nucleic acid sequence and/or modulation of expression of a target nucleic acid sequence to identify differences in activity of Cpf1 RNA-guided nucleases and/or gRNAs prepared by distinct manufacturing process. Such methods can also identify differences in activity of Cpf1 RNA-guided nucleases and/or gRNAs present in distinct formulations as well as those employing distinct delivery strategies.

In certain embodiments, the matched site target nucleic acid sequence is selected from the group consisting of Matched Site 1 (“MS1”; SEQ ID NO: 13), Matched Site 5 (“MS5”; SEQ ID NO: 14), Matched Site 11 (“MS11”; SEQ ID NO: 15), and Matched Site 18 (“MS18”; SEQ ID NO: 16). In certain embodiments, the matched site target nucleic acid sequence is MS5.

A variety of strategies can be employed to deliver the CRISPR/Cpf1 editing systems of the present disclosure to a cell. For example, but not by way of limitation, vector(s), e.g., AAV or other viral vectors, encoding the components of the CRISPR/Cpf1 editing system can be used to induce expression of the components of the CRISPR/Cpf1 editing system in the cell. Alternatively, RNP complexes comprising various components of the CRISPR/Cpf1 editing system can be delivered into a cell, e.g., by electroporation or any other suitable method which can be used for delivering RNP complexes into cells. In certain embodiments, lipid nanoparticles can be used to deliver RNP complexes into cells.

### BRIEF DESCRIPTION OF THE DRAWINGS

The accompanying drawings are intended to provide illustrative, and schematic rather than comprehensive, examples of certain aspects and embodiments of the present disclosure. The drawings are not intended to be limiting or binding to any particular theory or model, and are not necessarily drawn to scale. Without limiting the foregoing, nucleic acids and polypeptides may be depicted as linear sequences, or as schematic two- or three-dimensional structures; these depictions are intended to be illustrative rather than limiting or binding to any particular model or theory regarding their structure.

FIG. 1 provides a summary of how engineered Cpf1 variants expand the PAM targeting space.

FIG. 2 provides a summary of the sequences of four matched sites from Kleinstiver et al., Nature Biotechnology, 34(8):869-74 August 2016 (MS1, MS5, MS11, and MS18) and the cell types used in evaluating the performance of Cpf1 and Cas9 in connection with those match site target sequences.

FIGS. 3A-3B depict the results of a dose response experiment comparing increasing concentrations of Cpf1/gRNA RNPs to Cas9/gRNA RNPs at two matched site loci (MS1 and MS5) (FIG. 3A) as well as the results of an assay comparing the activity of AsCpf1 and SpCas9 on matched site targets MS1, MS5, MS11 and MS18, where Cpf1 edits certain target sites more efficiently than Cas9 (FIG. 3B).

FIG. 4 depicts a comparison of various AsCpf1 NLS variants across multiple cell types at a fixed 4.4 μM RNP dose with matched site 5 guide. The data is normalized to the variant displaying maximal editing for each cell type.

FIGS. 5A-5B depict a comparison of various two optimal AsCpf1 NLS variants at 4.4 μM RNP dose with guide RNA GWED545 targeting the TRAC locus in primary T cells (FIG. 5A) and a comparison of the His-AsCpf1-sNLS-sNLS variants at 4.4 μM RNP dose with guide RNA B2M-12 targeting the TRAC locus in primary T cells (FIG. 5B). In both instances, the data is normalized to variant displaying maximal editing.

FIG. 6 depicts the gRNA sequences employed in the HBG1 assays in HSCs and HUDEPs.

FIG. 7 depicts the gRNA sequences employed in the BCL11a assays in HSCs and HUDEPs.

FIG. 8 depicts specific sequences and their corresponding % editing of HBG1 or BCL11a in either HSCs or HUDEPs. Proposed gRNAs targeting HBB are also provided.

FIG. 9 depicts the HBG1 promoter region with gRNA AsCpf1 WT HBG1-1 binding at the CAAT box motif

FIG. 10 depicts a portion of the BCL11a enhancer region with gRNA BCL11a AsCpf1 RR-8 binding at the GATA1 motif.

FIG. 11 depicts the region of the HBG1 promoter screened using the gRNAs identified in FIG. 6. This region spans approximately 150 bp. HBG1-1 is shown overlapping with the CAAT box motif.

FIG. 12 depicts the region of the BCL11a erythroid enhancer screened using gRNAs identified in FIG. 7. This region spans approximately 600 base pairs and BCL11a RR-8 is shown overlapping with the GATA1 motif.

FIG. 13 depicts the cysteine mutants identified for the AsCpf1 Cysteine-low construct.

FIG. 14 depicts the results of an AlexaFluor maleimide assay demonstrating the significantly reduced accessibility of cysteine residues in AsCpf1 C334S C379S C674S.

FIG. 15 depicts a demonstration of equivalent endonuclease activity of WT AsCpf1, AsCpf1 no cysteines and two cysteine-low variants on MS5 substrate DNA.

FIG. 16 depicts the targeting of the HBG1 promoter region with AsCpf1 WT and RR PAM variant in HUDEPs and HSCs. The HUDEP experiment was performed with the optimal CA-137 pulse program and Lonza solution SE. The HSC screen was run with pulse code EO-100 and Lonza solution P3 as recommended by manufacturer. Dose was 4.4 μM RNP for all guides, with 2:1 guide:protein ratio. 50,000 HSCs were treated per condition. AsCpf1 WT and RR proteins had endotoxin levels of <5 EU/mL.

FIG. 17 depicts screening of the BCL11a enhancer region with AsCpf1 WT and RR and RVR PAM variants along with one WT FnCpf1 target in HUDEPs and HSCs. The HUDEP screen run was performed with the optimal CA-137 pulse program and Lonza solution SE. The HSC screen was run with pulse code EO-100 and Lonza solution P3 as recommended by manufacturer. Control guide for BCL11a (named KOBEH) shown as well. Dose was 4.4 μM RNP for all guides, with 2:1 guide:protein ratio. 50,000 HSCs were treated per condition. AsCpf1 WT, RR, and RVR proteins had endotoxin levels of <5 EU/mL.

FIG. 18 depicts nucleofection screening for AsCpf1 in HUDEPs. Dose was 2.2 μM AsCpf1 RNP using matched site 5 (MS5) guide RNA, at 2:1 guide:protein. AsCpf1 WT protein had endotoxin levels <5 EU/mL. Lonza solutions SE, SF and SG were tested with 50,000 HUDEPs/condition using different pulse programs. Pulse codes CA-137 and CA-138 with solution SE demonstrated optimal editing.

FIG. 19 depicts nucleofection screening for AsCpf1 in HSCs. Dose was 2.2 μM AsCpf1 RNP using matched site 5 (MS5) guide RNA, at 2:1 guide:protein. AsCpf1 WT protein had endotoxin levels <5 EU/mL. Lonza solutions P1, P2, P3, P4 and P5 were tested with 50,000 HSCs/condition using different pulse programs. Pulse codes CA-137 and CA-138 with solution P2 demonstrated optimal editing, as well as FF-100 and FF-104.

FIG. 20 depicts the use of a particular pulse code in Lonza Amaxa increases editing in HSCs across targets and PAM variants. Dose was 4.4 μM RNP for all guides, with 2:1 guide:protein ratio. 50,000 HSCs were treated per condition. AsCpf1 WT, RR, and RVR proteins had endotoxin levels of <5 EU/mL.

FIG. 21 depicts screening a T cell therapeutic target with AsCpf1 and its RR and RVR PAM variants at TRBC, TRAC and B2M loci. About 30% of gRNAs show more than 50% editing in the preliminary screen which was on par with generally observed SpCas9 hit rate, demonstrating that Cpf1 can potentially be used for gene editing on a patient's T cells at a therapeutic locus, including but not limited to, e.g., TRAC, TRBC and/or B2M.

FIG. 22 depicts that changes in the electroporation pulse code improve maximal editing significantly in T cells at multiple therapeutic target loci.

FIGS. 23A-23B depict efficient knockout editing in primary T cells at disease relevant loci with Cpf1 RNPs. FIG. 23A depicts RNP workflow for an ex vivo cellular therapy. FIG. 23B depicts efficient single KO at multiple therapeutically relevant T cell loci using AsCpf1 or an engineered PAM variant.

FIG. 24 depicts highly efficient double knockout of two therapeutic targets in T cells treated with Cpf1 RNP as measured by flow cytometry.

FIG. 25 depicts screening a T cell therapeutic target with AsCpf1 and its RR and RVR PAM variants at the TRBC, TRAC and B2M loci.

FIG. 26 summarizes high editing efficiency for AsCpf1 WT, RR, and RVR in T cells on three allogeneic T cell targets.

FIG. 27 illustrates the double knockout of two T cell targets with Cpf1 or Cas9 in human primary T cells.

FIG. 28 depicts screening a T cell therapeutic target with Cpf1 at the CIITA locus.

FIG. 29 summarizes high editing efficiency for Cpf1 in T cells on three allogeneic T cell targets, TRAC, CIITA and B2M, as compared to SpCas9.

FIG. 30 illustrates the efficiency for the triple knockout of three T cell targets with Cpf1 RNPs in T cells.

FIGS. 31A-31B. FIG. 31A summarizes the specificity of the top Cpf1 candidate guides for three T cell targets, CIITA, TRAC and B2M, and depicts the number of off-targets that were detected. FIG. 31B depicts that no detectable off-targets were found by targeted amplicon sequencing.

FIG. 32 depicts that the identification of electroporation conditions that improved maximal editing in T cells. Condition 1 was DS-130 and Condition 2 was CA-137.

FIG. 33 depicts the identification of NLS configuration that improved potency of gene editing in T cells. NLS v1 represents the sequence KRPAATKKAGQAKKKK (SEQ ID NO: 1) and NLS v2 represents the sequence 2×PKKKRKV (SEQ ID NO: 2).

FIG. 34 depicts the editing efficiency at the HBG-1 locus in HSCs using AsCpf1 with the HBG1-1 guide.

FIG. 35 depicts that editing efficiency of the NLS variants in T cells at matched-sited 5 using the MS5 guide RNA.

FIG. 36 depicts the reduction in MHC II in T cells that were edited at the CIITA locus as measured by flow cytometry.

FIG. 37A depicts the editing efficiency in T cells that were edited at the CIITA locus.

FIG. 37B depicts the genomic location that is targeted by the CIITA gRNAs CIITA-34, CIITA-41, CIITA-45 and CIITA-10.

FIG. 38 summarizes the percent reduction in MHC II in T cells that were edited at the CIITA locus.

FIG. 39 depicts the editing efficiency of Cpf1 CIITA gRNAs and depicts the number of off-targets that were detected for the gRNAs.

FIG. 40 depicts the editing efficiency of AspCpf1 RR and WT TRAC, CIITA and B2M gRNAs.

FIG. 41 depicts the editing efficiency of AspCpf1 RR and WT B2M gRNAs of different lengths.

FIG. 42 depicts the editing efficiency of AspCpf1 RR and WT TRAC gRNAs of different lengths.

FIG. 43 depicts the editing efficiency of AspCpf1 RR and WT CIITA gRNAs of different lengths.

FIG. 44A is a schematic representation of an unedited genomic DNA targeting site, an exemplary DNA donor template for targeted integration, potential insertion outcomes (i.e., non-targeted integration at the cleavage site or targeted integration at the cleavage site) and three potential PCR amplicons resulting from use of a primer pair targeting the P1 priming site and the P2 primer site (Amplicon X), a primer pair targeting the P1 primer site and the P2′ priming site (Amplicon Y), or a primer pair targeting the P1′ primer site and the P2 primer site (Amplicon Z). The depicted exemplary DNA donor template contains integrated primer sites (P1′ and P2′) and stuffer sequences (S1 and S2). A1/A2: donor homology arms, S1/S2: donor stuffer sequences, P1/P2: genomic primer sites, P1′/P2′: integrated primer sites, H1/H2: genomic homology arms, N: cargo, X: cleavage site.

FIG. 44B is a schematic representation of an unedited genomic DNA targeting site, an exemplary DNA donor template for targeted integration, potential insertion outcomes (i.e., non-targeted integration at the cleavage site or targeted integration at the cleavage site), and two potential PCR amplicons resulting from the use of a primer pair targeting the P1 primer site and the P2 primer site (Amplicon X), or a primer pair targeting the P1′ primer site and the P2 primer site (Amplicon Y). The exemplary DNA donor template contains an integrated primer site (P1′) and a stuffer sequence (S2). A1/A2: donor homology arms, S1/S2: donor stuffer sequences, P1/P2: genomic primer sites, P1′: integrated primer sites, H1/H2: genomic homology arms, N: cargo, X: cleavage site.

FIG. 44C is a schematic representation of an unedited genomic DNA targeting site, an exemplary DNA donor template for targeted integration, potential insertion outcomes (i.e., non-targeted integration at the cleavage site or targeted integration at the cleavage site), and two potential PCR amplicons resulting from the use of a primer pair targeting the P1 primer site and the P2 primer site (Amplicon X), or a primer pair targeting the P1 primer site and the P2′ primer site (Amplicon Y). The exemplary DNA donor template contains an integrated primer site (P2′) and a stuffer sequence (S1). A1/A2: donor homology arms, S1/S2: donor stuffer sequences, P1/P2: genomic primer sites, P2′: integrated primer sites, H1/H2: genomic homology arms, N: cargo, X: cleavage site.

FIG. 45 depicts exemplary DNA donor templates designed for gRNA targeting of the T cell receptor alpha constant (TRAC) locus.

FIG. 46 depicts gRNAs identified from a screening of the promoter region of HBG1 and HB G2.

### DETAILED DESCRIPTION

### Definitions and Abbreviations

Unless otherwise specified, each of the following terms has the meaning associated with it in this section.

The indefinite articles “a” and “an” refer to at least one of the associated noun, and are used interchangeably with the terms “at least one” and “one or more.” For example, “a module” means at least one module, or one or more modules.

The conjunctions “or” and “and/or” are used interchangeably as non-exclusive disjunctions.

The term “about” or “approximately,” as used herein, can mean within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” can mean an acceptable error range for the particular value, such as ±10% of the value modified by the term “about.”

The phrase “consisting essentially of” means that the species recited are the predominant species, but that other species may be present in trace amounts or amounts that do not affect structure, function or behavior of the subject composition. For instance, a composition that consists essentially of a particular species will generally comprise 90%, 95%, 96%, or more of that species.

“Domain” is used to describe a segment of a protein or nucleic acid. Unless otherwise indicated, a domain is not required to have any specific functional property.

An “indel” is an insertion and/or deletion in a nucleic acid sequence. An indel may be the product of the repair of a DNA double strand break, such as a double strand break formed by a genome editing system of the present disclosure. An indel is most commonly formed when a break is repaired by an “error prone” repair pathway such as the NHEJ pathway described below.

A “productive indel” with regards to HSCs refers to an indel (deletion and/or insertion) that results in HbF expression. In certain embodiments, a productive indel in an HSC may induce HbF expression. In certain embodiments, a productive indel in an HSC may result in an increased level of HbF expression. A “productive indel” with regards to T cells refers to an indel (deletion and/or insertion) that reduces the expression of a target gene in a T cell, e.g., an endogenous T cell gene. In certain embodiments, a “productive indel” in a T cell results in reducing or eliminating the expression of a cell surface protein or marker on the T cell.

“Gene conversion” refers to the alteration of a DNA sequence by incorporation of an endogenous homologous sequence (e.g. a homologous sequence within a gene array). “Gene correction” refers to the alteration of a DNA sequence by incorporation of an exogenous homologous sequence, such as an exogenous single- or double stranded donor template DNA. Gene conversion and gene correction are products of the repair of DNA double-strand breaks by HDR pathways such as those described below.

Indels, gene conversion, gene correction, and other genome editing outcomes are typically assessed by sequencing (most commonly by “next-gen” or “sequencing-by-synthesis” methods, though Saner sequencing may still be used) and are quantified by the relative frequency of numerical changes (e.g., ±1, ±2 or more bases) at a site of interest among all sequencing reads. DNA samples for sequencing may be prepared by a variety of methods known in the art, and may involve the amplification of sites of interest by polymerase chain reaction (PCR), the capture of DNA ends generated by double strand breaks, as in the GUIDEseq process described in Tsai et al. (Nat. Biotechnol. 34(5): 483 (2016), incorporated by reference herein) or by other means well known in the art. Genome editing outcomes may also be assessed by in situ hybridization methods such as the FiberComb™ system commercialized by Genomic Vision (Bagneux, France), and by any other suitable methods known in the art.

As used herein, the phrase “modification of a target sequence” as well as equivalents thereof, encompass, but are not limited to, the introduction of a deletion, an insertion, a gene conversion, a gene correction and/or an indel into the target sequence. A modification of a target sequence can result in an alteration of the expression of the target sequence, e.g., a modification to a coding sequence can disrupt expression of the protein encoded by that sequence, while modification of a regulatory sequence can result in increased or decreased expression of a protein under the control of that regulatory sequence, depending on whether the regulatory sequence activates or inhibits expression of the protein.

“Alt-HDR,” “alternative homology-directed repair,” or “alternative HDR” are used interchangeably to refer to the process of repairing DNA damage using a homologous nucleic acid (e.g., an endogenous homologous sequence, e.g., a sister chromatid, or an exogenous nucleic acid, e.g., a template nucleic acid). Alt-HDR is distinct from canonical HDR in that the process utilizes different pathways from canonical HDR, and can be inhibited by the canonical HDR mediators, RAD51 and BRCA2. Alt-HDR is also distinguished by the involvement of a single-stranded or nicked homologous nucleic acid template, whereas canonical HDR generally involves a double-stranded homologous template.

“Canonical HDR,” “canonical homology-directed repair” or “cHDR” refer to the process of repairing DNA damage using a homologous nucleic acid (e.g., an endogenous homologous sequence, e.g., a sister chromatid, or an exogenous nucleic acid, e.g., a template nucleic acid). Canonical HDR typically acts when there has been significant resection at the double strand break, forming at least one single stranded portion of DNA. In a normal cell, cHDR typically involves a series of steps such as recognition of the break, stabilization of the break, resection, stabilization of single stranded DNA, formation of a DNA crossover intermediate, resolution of the crossover intermediate, and ligation. The process requires RAD51 and BRCA2, and the homologous nucleic acid is typically double-stranded.

Unless indicated otherwise, the term “HDR” as used herein encompasses both canonical HDR and alt-HDR.

“Non-homologous end joining” or “NHEJ” refers to ligation mediated repair and/or non-template mediated repair including canonical NHEJ (cNHEJ) and alternative NHEJ (altNHEJ), which in turn includes microhomology-mediated end joining (MMEJ), single-strand annealing (SSA), and synthesis-dependent microhomology-mediated end joining (SD-MMEJ).

“Replacement” or “replaced,” when used with reference to a modification of a molecule (e.g., a nucleic acid or protein), does not require a process limitation but merely indicates that the replacement entity is present.

“Subject” means a human or non-human animal. A human subject can be any age (e.g., an infant, child, young adult, or adult), and may suffer from a disease, or may be in need of modification of a gene. Alternatively, the subject may be an animal, which term includes, but is not limited to, mammals, birds, fish, reptiles, amphibians, and more particularly non-human primates, rodents (such as mice, rats, hamsters, etc.), rabbits, guinea pigs, dogs, cats, and so on. In certain embodiments of this disclosure, the subject is livestock, e.g., a cow, a horse, a sheep or a goat. In certain embodiments, the subject is poultry. In certain embodiments, the subject is a plant.

“Treat,” “treating,” and “treatment” mean the treatment of a disease in a subject (e.g., a human subject), including one or more of inhibiting the disease, i.e., arresting or preventing its development or progression; relieving the disease, i.e., causing regression of the disease state; relieving one or more symptoms of the disease; and curing the disease.

“Prevent,” “preventing,” and “prevention” refer to the prevention of a disease in a mammal, e.g., in a human, including (a) avoiding or precluding the disease; (b) affecting the predisposition toward the disease; or (c) preventing or delaying the onset of at least one symptom of the disease.

A “kit” refers to any collection of two or more components that together constitute a functional unit that can be employed for a specific purpose. By way of illustration (and not limitation), one kit according to this disclosure can include a guide RNA complexed or able to complex with an RNA-guided nuclease, and accompanied by (e.g., suspended in, or suspendable in) a pharmaceutically acceptable carrier. The kit can be used to introduce the complex into, for example, a cell or a subject, for the purpose of causing a desired genomic alteration in such cell or subject. The components of a kit can be packaged together, or they may be separately packaged. Kits according to this disclosure also optionally include directions for use (DFU) that describe the use of the kit e.g., according to a method of this disclosure. The DFU can be physically packaged with the kit, or it can be made available to a user of the kit, for instance by electronic means.

The terms “polynucleotide”, “nucleotide sequence”, “nucleic acid”, “nucleic acid molecule”, “nucleic acid sequence”, and “oligonucleotide” refer to a series of nucleotide bases (also called “nucleotides”) in DNA and RNA, and mean any chain of two or more nucleotides. The polynucleotides, nucleotide sequences, nucleic acids etc. can be chimeric mixtures or derivatives or modified versions thereof, single-stranded or double-stranded. They can be modified at the base moiety, sugar moiety, or phosphate backbone, for example, to improve stability of the molecule, its hybridization parameters, etc. A nucleotide sequence typically carries genetic information, including, but not limited to, the information used by cellular machinery to make proteins and enzymes. These terms include double- or single-stranded genomic DNA, RNA, any synthetic and genetically manipulated polynucleotide, and both sense and antisense polynucleotides. These terms also include nucleic acids containing modified bases.

Conventional IUPAC notation is used in nucleotide sequences presented herein, as shown in Table 1, below (see also Cornish-Bowden A, Nucleic Acids Res. 1985 May 10; 13(9):3021-30, incorporated by reference herein). It should be noted, however, that “T” denotes “Thymine or Uracil” in those instances where a sequence may be encoded by either DNA or RNA, for example in gRNA targeting domains.

TABLE 1
IUPAC nucleic acid notation

| Character | Base |
|---|---|
| A | Adenine |
| T | Thymine or Uracil |
| G | Guanine |
| C | Cytosine |
| U | Uracil |
| K | G or T/U |
| M | A or C |
| R | A or G |
| Y | C or T/U |
| S | C or G |
| W | A or T/U |
| B | C, G or T/U |
| V | A, C or G |
| H | A, C or T/U |
| D | A, G or T/U |
| N | A, C, G or T/U |

The terms “protein,” “peptide” and “polypeptide” are used interchangeably to refer to a sequential chain of amino acids linked together via peptide bonds. The terms include individual proteins, groups or complexes of proteins that associate together, as well as fragments or portions, variants, derivatives and analogs of such proteins. Peptide sequences are presented herein using conventional notation, beginning with the amino or N-terminus on the left, and proceeding to the carboxyl or C-terminus on the right. Standard one-letter or three-letter abbreviations can be used.

The term “variant” refers to an entity such as a polypeptide, polynucleotide or small molecule that shows significant structural identity with a reference entity (e.g., a wild-type or naturally occurring entity) but differs structurally from the reference entity in the presence or level of one or more chemical moieties, e.g., an amino acid in the context of polypeptide or a nucleotide in the context of a polynucleotide, as compared with the reference entity. The term variant, as used herein, also encompasses an entity such as a polypeptide, a polynucleotide or small molecule which is functionally better or superior than a reference entity, in one or more properties associated with such an entity. In many embodiments, a variant also differs functionally from its reference entity. For example, by not by limitation, a “variant Cpf1 polypeptide” encompasses an AsCpf1 variant comprising the S542R/K607R substitution, and which recognizes TYCV PAM, as well as the AsCpf1 variant comprising the S542R/K548V/N552R substitution, and which recognizes TATV PAM.

As used herein, the term “cleavage event” refers to a break in a nucleic acid molecule. A cleavage event may be a single-strand cleavage event, or a double-strand cleavage event. A single-strand cleavage event may result in a 5′ overhang or a 3′ overhang. A double-stranded cleavage event may result in blunt ends, two 5′ overhangs, or two 3′ overhangs.

The term “cleavage site,” as used herein in reference to a site on a target nucleic acid sequence, refers to a target position between two nucleotide residues of the target nucleic acid where a double-stranded break occurs, or alternatively, to a target position within a span of several nucleotide residues of the target nucleic acid wherein two single stranded breaks occur, as mediated by a RNA-guided nuclease-dependent process. A cleavage site may be the target position for, e.g., a blunt double stranded break. Alternatively, a cleavage site may be a target site within a span of several nucleotide residues of the target nucleic acid for, e.g., two single strand breaks or nicks which form a double strand break and which are separated by, e.g., about 10 base pairs. The double strand break(s) or the closer of the two single strand nicks in a pair will ideally be within 0-500 bp of a target position (e.g., no more than 450, 400, 350, 300, 250, 200, 150, 100, 50, or 25 bp from the target position). When dual nickases are used, the two nicks in a pair are within 25-55 bp of each other (e.g., between 25 and 50, 25 and 45, 25 and 40, 25 and 35, 25 and 30, 50 and 55, 45 and 55, 40 and 55, 35 and 55, 30 and 55, 30 and 50, 35 and 50, 40 and 50, 45 and 50, 35 and 45, or 40 and 45 bp) and no more than 100 bp away from each other (e.g., no more than 90, 80, 70, 60, 50, 40, 30, 20, or 10 bp).

### Overview

The present disclosure provides CRISPR/Cpf1-related methods and components for the editing of a target nucleic acid sequence and/or modulating expression of a target nucleic acid sequence. For example, the present disclosure provides CRISPR/Cpf1-related methods for targeting nucleic acid sequences that affect hematopoietic stem cell (HSC) proliferation, survival, persistence and/or function. In certain non-limiting embodiments, the present disclosure provides the first evidence of efficient editing of a target nucleic acid sequence in CD34⁺ cells by a Cpf1 RNA-guided nuclease. Further, the present disclosure provides the first evidence of efficient editing by a Cpf1 RNA-guided nuclease of genes, BCL11a and HBG1, associated with hereditary persistence of fetal hemoglobin (referred to herein as “HPFH”). The present disclosure also provides CRISPR/Cpf1-related methods for targeting nucleic acid sequences that affect T cell proliferation, survival, persistence and/or function. The present disclosure further provides modified Cpf1 proteins that exhibit significant editing efficiency and exhibit improved properties, and strategies for evaluating the efficiency of such modified Cpf1 proteins.

Modified Cpf1 Proteins

In one aspect, the present disclosure relates to modified Cpf1 proteins and their use in CRISPR/Cpf1-related methods for editing a target nucleic acid sequence and/or modulating expression of a target nucleic acid sequence.

In certain embodiments, the modified Cpf1 protein is derived from a Cpf1 protein selected from the group consisting of Acidaminococcus sp. strain BV3L6 Cpf1 protein (AsCpf1), Francisella novicida U112 (FnCpf1), Moraxella bovoculi 237 (MbCpf1), Candidatus Methanomethylphilus alvus Mx1201 (CMaCpf1), Sneatia amnii (SaCpfq), Moraxella lacunata (M1Cpf1), Moraxella bovoculi AAX08_00205 (Mb2Cpf1), Moraxella bovoculi AAX11_00205 (Mb3Cpf1), Lachnospiraceae bacterium ND2006 Cpf1 protein (LbCpf1), Lachnospiraceae bacterium MA2020 (Lb5Cpf1), Lachnospiraceae bacterium MC2017 (Lb4Cpf1), Flavobacterium brachiophilum FL-15 (FbCpf1), Thiomicrospira sp. XS5 (TsCpf1), Parcubacteria group bacterium GW2011 (PgCpf1), Candidatus Roizmanbacteria bacterium GW2011 (CRbCpf1), Candidatus Peregrinbacteria bacterium GW2011 (CPbCpf1), Btyrivibrio sp. NC3005 (BsCpf1), Butyrivibrio fibrisolvens (BfCpf1), Prevotella bryantii B14 (Pb2Cpf1) and Bacteroidetes oral taxon 274 (BoCpf1) (see, e.g., Zetsche et al., bioRxiv 134015; doi: https://doi.org/10.1101/134015, the contents of which is incorporated by reference herein in its entirety). In certain embodiments, the Cpf1 protein comprises a sequence selected from the group consisting of SEQ ID NOs: 17-19, having the codon-optimized nucleic acid sequences of SEQ ID NOs: 20-22, respectively.

Cpf1 Nuclear Localization Signal (NLS) Variants

In certain embodiments, the modified Cpf1 protein comprises a nuclear localization signal (NLS) (also referred to herein as “Cpf1 NLS variants”). For example, but not by way of limitation, NLS sequences useful in connection with the methods and compositions disclosed herein will comprise an amino acid sequence capable of facilitating protein import into the cell nucleus. NLS sequences useful in connection with the methods and compositions disclosed herein are known in the art. Non-limiting examples of such NLS sequences include the nucleoplasmin NLS having the amino acid sequence: KRPAATKKAGQAKKKK (SEQ ID NO: 1) and the simian virus 40 “SV40” NLS having the amino acid sequence PKKKRKV (SEQ ID NO: 2).

In certain embodiments, the modified Cpf1 protein can have one or more, e.g., two or more, three or more or four or more, NLS sequences. For example, but not by way of limitation, the modified Cpf1 protein can have two NLS sequences, three NLS sequences or four NLS sequences. In certain embodiments, the modified Cpf1 protein can have two NLS sequences. In certain embodiments, the NLS sequence of the modified Cpf1 protein is positioned at or near the C-terminus of the Cpf1 protein sequence. In certain embodiments, the NLS sequence of the modified Cpf1 protein is positioned at or near the N-terminus of the Cpf1 protein sequence. In certain embodiments, a modified Cpf1 protein of the present disclosure can have one or more NLS sequences positioned at or near the N-terminus of the Cpf1 protein sequence and one or more NLS sequences positioned at or near the C-terminus of the Cpf1 protein sequence, e.g., the modified Cpf1 protein comprises NLS sequences positioned at or near both the N-terminus and C-terminus of the Cpf1 protein sequence.

In certain embodiments, a modified Cpf1 protein having an NLS sequence positioned at or near the C-terminus of the Cpf1 protein sequence can be selected from the following: His-AsCpf1-nNLS (also referred to herein as “Asp Cpf1 NLS v1”) (SEQ ID NO: 3); His-AsCpf1-sNLS (SEQ ID NO: 4); and His-AsCpf1-sNLS-sNLS (also referred to herein as “Asp Cpf1 NLS v2”) (SEQ ID NO: 5), where “His” refers to a six-histidine purification sequence, “AsCpf1” refers to the Acidaminococcus sp. Cpf1 protein sequence, “nNLS” refers to the nucleoplasmin NLS, and “sNLS” refers to the SV40 NLS. Additional permutations of the identity and C-terminal positions of NLS sequences, e.g., appending two or more nNLS sequences or combinations of nNLS and sNLS sequences (or other NLS sequences), as well as sequences with and without purification sequences, e.g., six-histidine sequences, are within the scope of the instantly disclosed subject matter.

In certain embodiments, a modified Cpf1 protein having an NLS sequence positioned at or near the N-terminus of the Cpf1 protein sequence can be selected from the following: His-sNLS-AsCpf1 (SEQ ID NO: 6), His-sNLS-sNLS-AsCpf1 (SEQ ID NO: 7), and sNLS-sNLS-AsCpf1 (SEQ ID NO: 8). Additional permutations of the identity and N-terminal positions of NLS sequences, e.g., appending two or more nNLS sequences or combinations of nNLS and sNLS sequences (or other NLS sequences), as well as sequences with and without purification sequences, e.g., six-histidine sequences, are within the scope of the instantly disclosed subject matter.

In certain embodiments, a modified Cpf1 protein having NLS sequences positioned at or near both the N-terminus and C-terminus of the Cpf1 protein sequence can be selected from the following: His-sNLS-AsCpf1-sNLS (SEQ ID NO: 9) and His-sNLS-sNLS-AsCpf1-sNLS-sNLS (SEQ ID NO: 10). Additional permutations of the identity and N-terminal/C-terminal positions of NLS sequences, e.g., appending two or more nNLS sequences or combinations of nNLS and sNLS sequences (or other NLS sequences) to either the N-terminal/C-terminal positions, as well as sequences with and without purification sequences, e.g., six-histidine sequences, are within the scope of the instantly disclosed subject matter.

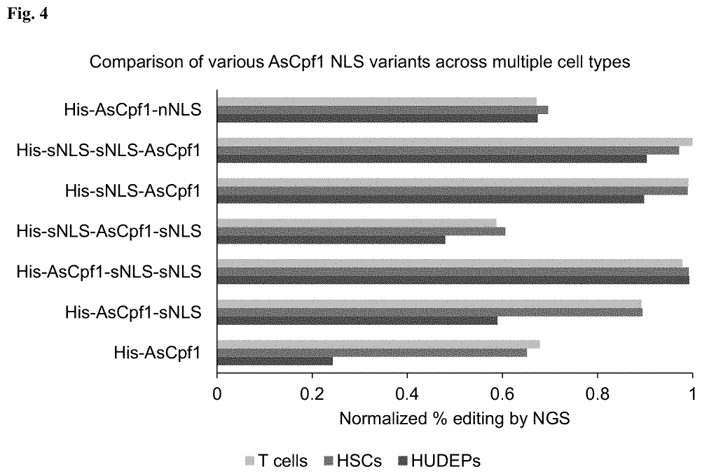

To determine the Cpf1 protein modifications, e.g., NLS modifications, advantageous for editing in CD34⁺ cells and T cells, AsCpf1 proteins were synthesized containing different locations and types of NLS sequences. The protein variants were complexed to Matched Site 5 targeting gRNAs and electroporated into CD34⁺ cells, T cells, and HUDEPs (4.4 μM RNP). In FIG. 4, the results are depicted as % editing normalized to the variant displaying maximal editing for each cell type. The data indicate that different species of nucleases have variable activity at the same target site in CD34⁺ cells and T cells (among other cells) and efficient editing by AsCpf1 can be achieved in CD34⁺ cells and T cells (among other cells).

Cysteine-Modified Cpf1 Proteins and RNPs

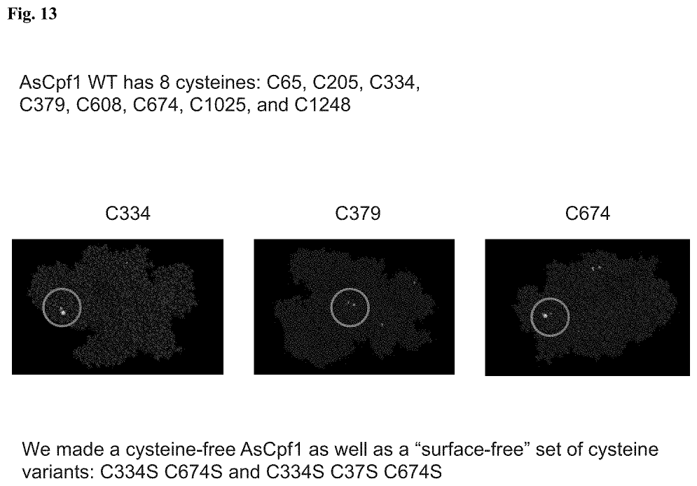

Disulfide bond formation is known to promote protein aggregation. Accordingly, the Cpf1 crystal structure and the known Cpf1 primary amino acid sequence were analyzed in an effort to identify cysteines that could be altered to reduce the possibility of such disulfide bond formation (FIG. 13).

The modified Cpf1 proteins of the present disclosure can comprise an alteration (e.g., a deletion or substitution) at one or more cysteine residues of the Cpf1 protein sequence. Such modified Cpf1 proteins exhibit reduced aggregation, which is especially useful when scaling up manufacturing of the protein. For example, but not by way of limitation, a modified Cpf1 protein comprises an alteration at one or more positions, e.g., two or more, three or more, four or more, five or more, six or more, seven or more or eight positions, selected from the group consisting of: C65, C205, C334, C379, C608, C674, C1025, and C1248. In certain embodiments, the modified Cpf1 protein comprises a substitution of one or more cysteine residues for a serine or alanine. In certain embodiments, the modified Cpf1 protein comprises one or more alterations, e.g., substitutions, selected from the group consisting of: C65S, C2055, C334S, C379S, C608S, C674S, C1025S, and C1248S. In certain embodiments, the modified Cpf1 protein comprises one or more alterations selected from the group consisting of: C65A, C205A, C334A, C379A, C608A, C674A, C1025A and C1248A. In certain embodiments, the modified Cpf1 protein comprises alterations at positions C334 and C674 or C334, C379, and C674. In certain embodiments, the modified Cpf1 protein comprises the following alterations: C334S and C674S, or C334S, C379S, and C674S. In certain embodiments, the modified Cpf1 protein comprises the following alterations: C334A and C674A, or C334A, C379A and C674A. In certain embodiments, the modified Cpf1 protein comprises both one or more cysteine residue alterations as well as the introduction of one or more NLS sequences, e.g., His-AsCpf1-nNLS Cys-less (SEQ ID NO: 11) or His-AsCpf1-nNLS Cys-low (SEQ ID NO: 12), as described herein.

Cpf1 Editing of CD34⁺ HSCs at Target Sites Associated with Hemoglobinopathies

The present disclosure further provides CRISPR/Cpf1-related methods for editing a target nucleic acid sequence for treating hemoglobinopathies, e.g., beta thalassemia and sickle cell disease. For example, but not by way of limitation, the CRISPR/Cpf1-related methods result in the disruption of one or more genes in CD34⁺ cells that regulate the expression of Fetal hemoglobin (HbF).

One therapeutic strategy for treating hemoglobinopathies involves inducing an increase in the expression of HbF. HbF expression can be induced via the targeted disruption of the erythroid cell specific expression of a transcriptional repressor, BCL11a (Canvers et al., Nature, 527(12): 192-197). One strategy to increase HbF expression is to employ gene editing disrupt BCL11a expression. For example, but not by way of limitation, an RNA-guided nuclease, e.g., Cpf1 RNA-guided nuclease, can target a particular target sequence influencing expression of the BCL11a gene. In certain embodiments, any region of the BCL11a gene can be targeted.

The present disclosure provides a cell or a population of cells that include a modification in the BCL11a gene, e.g., to disrupt, knockdown or knockout BCL11a expression. For example, but not by way of limitation, the cell or population of cells can be generated by the delivery of a complex comprising a Cpf1 RNA-guided nuclease and a gRNA molecule, e.g., an RNP complex, that targets the BCL11a gene sequence. In certain embodiments, at least about 5%, at least about 10%, at least about 20%, at least about 30%, at least about 40%, at least about 50%, at least about 60%, at least about 70%, at least about 80% or at least about 90% of the cells in the population of cells modified. In certain embodiments, at least about 5%, at least about 10%, at least about 20%, at least about 30%, at least about 40%, at least about 50%, at least about 60%, at least about 70%, at least about 80% or at least about 90% of the cells in the population of cells have a productive indel.

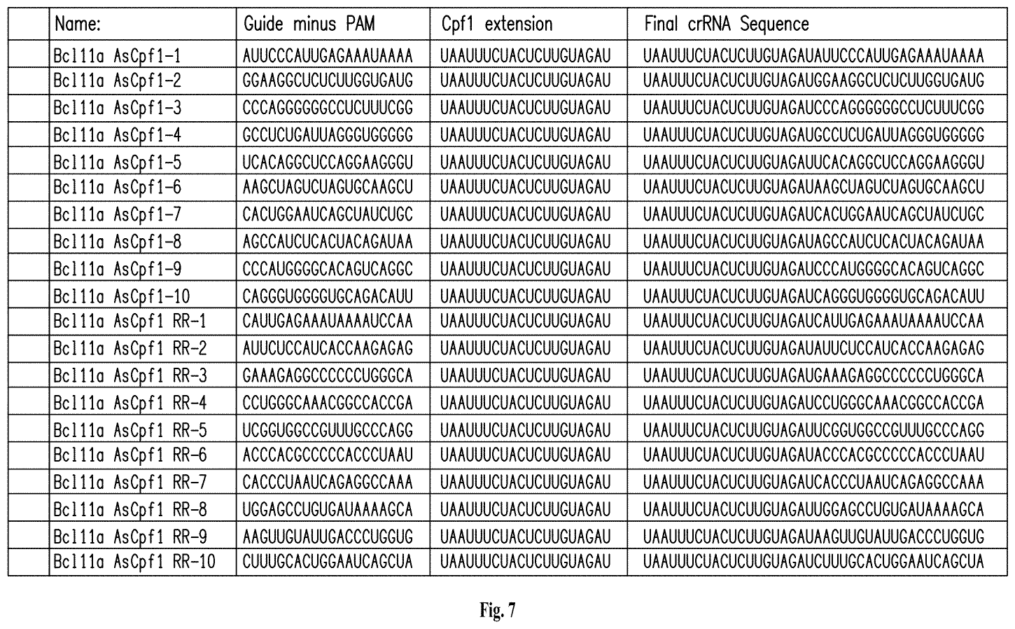

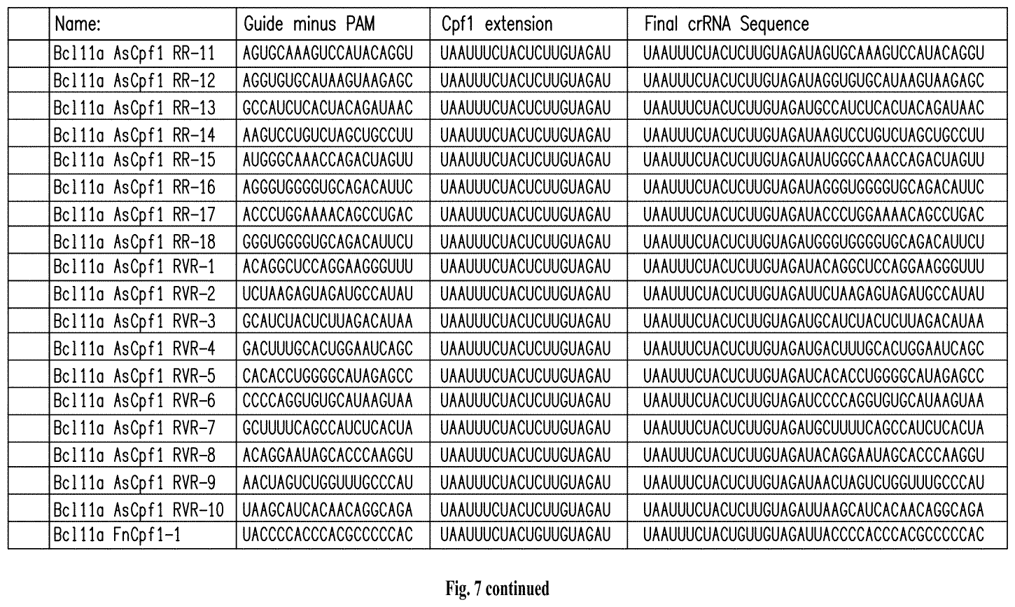

In certain embodiments, the Cpf1 RNA-guided nuclease can target intron 2 of the BCL11a gene. In certain embodiments, the Cpf1 RNA-guided nuclease will be targeted to disrupt the GATA1 binding motif in the erythroid specific enhancer of BCL11a that is in the +58 DHS region of intron 2 of the BCL11a gene. Exemplary gRNA molecules for use in such a CRISPR/Cpf1 editing system targeting BCL11a are identified in FIGS. 7, 10 and 12.

In certain embodiments, the instant disclosure is directed to a cell where the BCL11a gene is disrupted. In certain embodiments, the erythroid enhancer region of the BCL11a gene can be targeted, e.g., the erythroid enhancer region between +55 kb and +62 kb from the Transcription Start Site (TSS). For example, but not by way of limitation, the present disclosure is directed to a cell where the +58 DHS region of intron 2 of the BCL11a gene is disrupted. In certain embodiments, such a cell can include one or more components of a CRISPR/Cpf1 editing system. In certain embodiments, the instant disclosure is directed to a population of cells where the BCL11a gene is disrupted, e.g., where the +58 DHS region of intron 2 of the BCL11a gene is disrupted. In certain embodiments, such a cell population comprises cells comprising one or more components of a CRISPR/Cpf1 editing system. In certain embodiments, the instant disclosure is directed to a cell wherein the GATA1 motif of the BCL11a gene is disrupted. In certain embodiments, such a cell can include one or more components of a CRISPR/Cpf1 editing system. In certain embodiments, the instant disclosure is directed to a population of cells wherein the GATA1 motif of the BCL11a gene is disrupted. In certain embodiments, such a cell population can include cells comprising one or more components of a CRISPR/Cpf1 editing system.

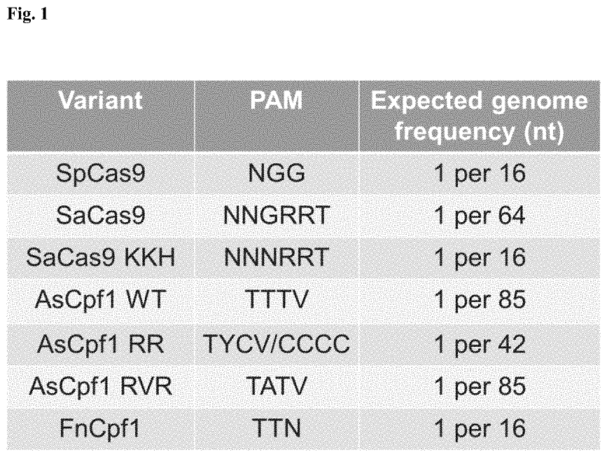

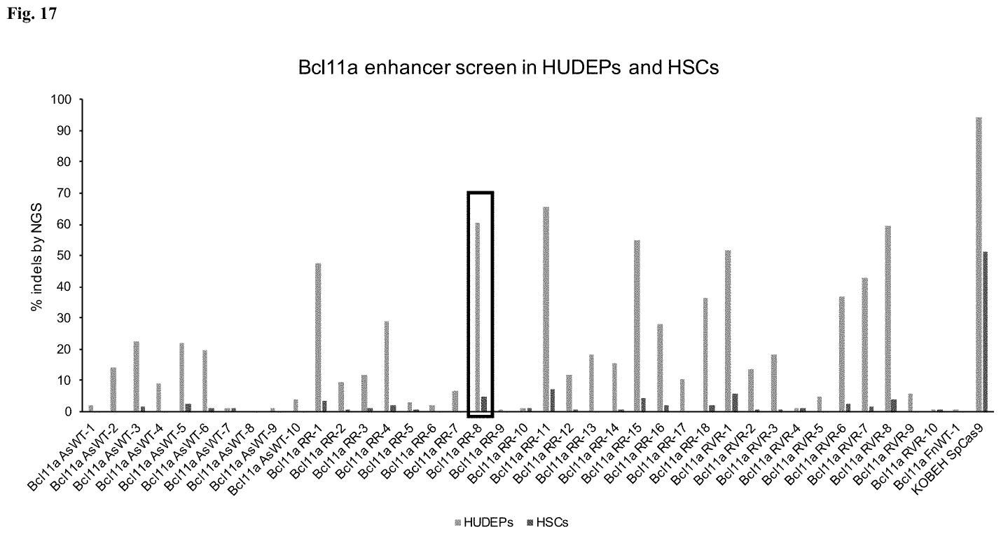

As outlined in Example 3, below, AsCpf1 successfully mediated editing of target sites in the +58 DHS region of intron 2 of the BCL11a gene. First, a number of AsCpf1 variant guide RNAs with different PAMs (FIG. 1) were screened in HUDEP2 cells and then the most efficient guide RNAs and nuclease variants were tested in mPB CD34⁺ cells (FIG. 17). In particular, FIG. 17 depicts screening of the BCL11a enhancer region with AsCpf1 WT and RR and RVR PAM variants along with one WT FnCpf1 target in HUDEPs and HSCs.

Another strategy to induce the expression of fetal hemoglobin in connection with the treatment of hemoglobinopathies, e.g., beta thalassemia and sickle cell disease, is to disrupt the expression of the HBG locus, and in particular the expression of HGB1 and/or HGB2.

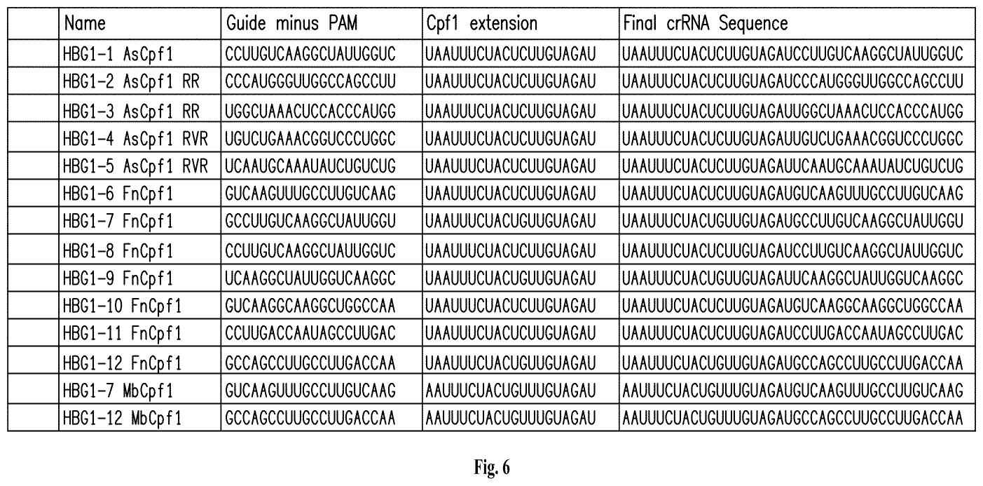

In certain embodiments, the instant disclosure relates to the use of CRISPR/Cpf1-mediated editing of the HBG locus. In certain embodiments, any region of the HBG locus can be targeted. In certain embodiments, CRISPR/Cpf1-mediated editing, as described herein, can be employed to disrupt a non-coding region of the HBG locus (see, e.g., Table 18). In certain embodiments, CRISPR/Cpf1-mediated editing, as described herein, can be employed to disrupt an intron of the HBG locus. In certain embodiments, CRISPR/Cpf1-mediated editing, as described herein, can be employed to disrupt a cis-regulatory region of the HBG gene is targeted. For example, but not by way of limitation, a cis-regulatory region can include a promoter and/or an enhancer. In certain embodiments, the instant disclosure relates to the use of CRISPR/Cpf1-mediated editing of the promoter region of the HBG locus. In certain embodiments, CRISPR/Cpf1-mediated editing, as described herein, can be employed to disrupt a region between −800 and −60 nt of the promoter region of the HBG locus. For example, but not by way of limitation, CRISPR/Cpf1-mediated editing can be employed to disrupt the −110 nt promoter region of the HBG promoter region and/or the CAAT box present in the HBG promoter region. Disruption of the HBG promoter region generally and the CAAT box specifically can be accomplished via the delivery of a CRISPR/Cpf1 editing system targeted to those sequences. Exemplary gRNA molecules for use in such a CRISPR/Cpf1 editing system targeting those sequences of the HBG locus are identified in FIGS. 6, 9 and 11 and Table 19. Chromosomal regions (e.g., genomic coordinates) that can be targeted to disrupt an HBG locus is provided in Table 18. In certain embodiments, the gRNA molecule for use in disrupting the HBG1 locus is HBG1-1.

The present disclosure provides a cell or a population of cells that include a modification in the HBG locus, e.g., to disrupt, knockdown or knockout HBG expression. For example, but not by way of limitation, the cell or population of cells can be generated by the delivery of a complex comprising a Cpf1 RNA-guided nuclease and a gRNA molecule, e.g., an RNP complex, that targets the HBG locus. In certain embodiments, at least about 5%, at least about 10%, at least about 20%, at least about 30%, at least about 40%, at least about 50%, at least about 60%, at least about 70%, at least about 80% or at least about 90% of the cells in the population of cells are modified. In certain embodiments, at least about 5%, at least about 10%, at least about 20%, at least about 30%, at least about 40%, at least about 50%, at least about 60%, at least about 70%, at least about 80% or at least about 90% of the cells in the population of cells have a productive indel.

In certain embodiments, the instant disclosure is directed to a cell, e.g., a CD34+ hematopoietic stem and progenitor cell, where the HBG locus is disrupted. For example, but not by way of limitation, the present disclosure is directed to a cell where the promoter region of the HBG locus is disrupted. In certain embodiments, the −110 nt promoter region of the HBG locus is disrupted. In certain embodiments, such a cell can include one or more components of a CRISPR/Cpf1 editing system. In certain embodiments, the instant disclosure is directed to a population of cells wherein the −110 nt promoter region of the HBG locus is disrupted. In certain embodiments, such a cell population can include cells comprising one or more components of a CRISPR/Cpf1 editing system, as determined using suitable methods to detect such components. In certain embodiments, the instant disclosure is directed to a cell wherein the CAAT box present in the HBG promoter region is disrupted. In certain embodiments, such a cell comprises one or more components of a CRISPR/Cpf1 editing system. In certain embodiments, the instant disclosure is directed to a population of cells wherein the CAAT box present in the HBG promoter region is disrupted. In certain embodiments, such a cell population can include cells comprising one or more components of a CRISPR/Cpf1 editing system, as determined using suitable methods to detect such components. In certain embodiments, the present disclosure provides a population of cells where the HBG1 locus is disrupted by the use of a CRISPR/Cpf1 editing system that includes the gRNA HBG1-1.

In certain embodiments, a CRISPR/Cpf1-edited cell or population of CRISPR/Cpf1-edited cells that include a modification in the HBG locus or the BCL11a gene do not include one or more components of a CRISPR/Cpf1 editing system, as determined using suitable methods to detect such components. In certain embodiments, less than about 10%, less than about 9%, less than about 8%, less than about 7%, less than about 6%, less than about 5%, less than about 4%, less than about 3%, less than about 2% or less than about 1% of the cells in the population of CRISPR/Cpf1-edited cells include one or more components of a CRISPR/Cpf1 editing system, as determined using suitable methods to detect such components. In certain embodiments, the present disclosure provides a population of CRISPR/Cpf1-edited cells that are to administered to a subject in need thereof, where less than about 10%, less than about 9%, less than about 8%, less than about 7%, less than about 6%, less than about 5%, less than about 4%, less than about 3%, less than about 2% or less than about 1% of the cells in the CRISPR/Cpf1-edited cell population include one or more components of a CRISPR/Cpf1 editing system.

In certain embodiments, the disruption of the BCL11a gene or an HBG gene in a cell by a CRISPR/Cpf1 editing system of the present disclosure can result in an increase in the expression of fetal hemoglobin in the cell as compared to a cell that does not have a disruption in the BCL11a gene or an HBG gene. For example, but not by way of limitation, expression of fetal hemoglobin can be increased by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% relative to the level of expression of fetal hemoglobin in a cell that does not have a disruption in the BCL11a gene or an HBG locus and/or gene.

In certain embodiments, the disruption of the BCL11a gene or an HBG gene in a cell by a CRISPR/Cpf1 editing system of the present disclosure can result in an increase in the expression of fetal hemoglobin in an amount suitable to partially or completely alleviate the symptoms of a hemoglobinopathy, e.g., sickle cell disease or beta-thalassemia. For example, but not by way of limitation, the increase in the expression of fetal hemoglobin can be greater than about 1 picogram (pg), greater than about 2 pg, greater than about 3 pg, greater than about 4 pg, greater than about 5 pg, greater than about 6 pg, greater than about 7 pg, greater than about 8 pg, greater than about 9 pg, greater than about 10 pg, greater than about 11 pg, greater than about 12 pg, greater than about 13 pg, greater than about 14 pg or greater than about 15 pg.

In certain embodiments, the disruption of the BCL11a gene or an HBG gene in a cell by a CRISPR/Cpf1 editing system of the present disclosure can result in the production of at least about 1 picogram, at least about 2 picograms, at least about 3 picograms, at least about 4 picograms, at least about 5 picograms, at least about 6 picograms, at least about 7 picograms, at least about 8 picograms, at least about 9 picograms, at least about 10 picograms, or from about 8 to about 9 picograms or from about 9 to about 10 picograms fetal hemoglobin per cell.

The disclosure also relates to a population of cells modified by the genome editing system described above, wherein a higher percentage of the population of cells are capable of differentiating into a population of cells of an erythroid lineage that express HbF relative to a population of cells not modified by the genome editing system. In certain embodiments, the higher percentage may be at least about 15%, at least about 20%, at least about 25%, at least about 30% or at least about 40% higher. In certain embodiments, the cells may be hematopoietic stem cells. In certain embodiments, the cells may be capable of differentiating into an erythroblast, erythrocyte, or a precursor of an erythrocyte or erythroblast.

In certain embodiments, the expression levels, e.g., relative expression levels of HbF (e.g., over total beta-like globin chains) can be measured by ultra performance liquid chromatography (UPLC).

A variety of strategies can be employed to deliver the CRISPR/Cpf1 editing systems of the present disclosure to a cell. For example, but not by way of limitation, vector(s), e.g., AAV or other viral vectors, encoding the components of the CRISPR/Cpf1 editing system can be used to induce expression of the components of the CRISPR/Cpf1 editing system in the cell. Alternatively, RNP complexes comprising the components of the CRISPR/Cpf1 editing system can be introduced, e.g., via electroporation, into the cell. In certain embodiments, the RNP complexes can be delivered by lipid nanoparticles into the cell.

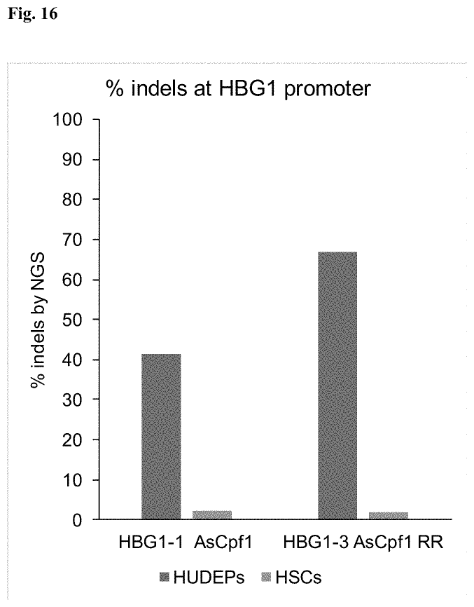

As outlined in Example 3, below, FIG. 16 depicts the successful targeting of the HBG1 promoter region with AsCpf1 WT and RR PAM variant in HUDEPs and HSCs.

Together, these data relating to the disruption of the BCL11a gene and the HBG locus show efficient editing by AsCpf1 variants in CD34⁺ cells at clinically relevant loci (i.e., known HPFH target sites).

Cpf1 Editing of T Cells at Target Sites Associated with T Cell Proliferation, Survival and/or Function

One therapeutic strategy proposed for treating cancer involves adoptive T cell transfer. Factors limiting the efficacy of genetically modified T cells as cancer therapeutics include (1) T cell proliferation, e.g., limited proliferation of T cells following adoptive transfer; (2) T cell survival, e.g., induction of T cell apoptosis by factors in the tumor environment; and (3) T cell function, e.g., inhibition of cytotoxic T cell function by inhibitory factors secreted by host immune cells and cancer cells. One strategy to increase efficacy is to employ gene editing to modify or disrupt T cell genes associated to T cell proliferation, survival and/or function. For example, but not by way of limitation, an RNA-guided nuclease, e.g., Cpf1 RNA-guided nuclease, can target a particular sequence influencing expression of the T cell genes.

Methods and compositions encompassed by the present disclosure can be used to affect T cell proliferation, survival, persistence, and/or function by modifying one or more T cell expressed gene(s), e.g., one or more of FAS, BID, CTLA4, PDCD1, CBLB, PTPN6, B2M, TRAC, CIITA and TRBC genes. In certain embodiments, methods and compositions disclosed herein can be used to affect T cell proliferation by modifying one or more T cell expressed gene, e.g., the CBLB and/or PTPN6 gene. In certain embodiments, methods and compositions disclosed herein can be used to affect T cell survival by modifying one or more T cell expressed gene, e.g., FAS and/or BID gene. In certain embodiments, methods and compositions disclosed herein can be used to affect T cell function by modifying one or more T cell expressed gene, e.g., CTLA4, PDCD1, TRAC, CIITA and/or TRBC gene. In certain embodiments, methods and compositions disclosed herein can be used to improve T cell persistence by modifying the B2M gene.

In certain embodiments, one or more T cell expressed gene, including, but not limited to, FAS, BID, CTLA4, PDCD1, CBLB, PTPN6, B2M, TRAC, CIITA and TRBC genes, are independently targeted as a targeted knockout, e.g., to influence T cell proliferation, survival, persistence and/or function. In certain embodiments, a presently disclosed method comprises knocking out one T cell expressed gene (e.g., one selected from the group consisting of FAS, BID, CTLA4, PDCD1, CBLB, PTPN6, B2M, TRAC, CIITA and TRBC genes). In certain embodiments, a presently disclosed method comprises independently knocking out two T cell expressed genes (e.g., two selected from the group consisting of FAS, BID, CTLA4, PDCD1, CBLB, PTPN6, B2M, TRAC, CIITA and TRBC genes). In certain embodiments, a presently disclosed method comprises independently knocking out three T cell expressed genes, e.g., three selected from the group consisting of FAS, BID, CTLA4, PDCD1, CBLB, PTPN6, B2M, TRAC, CIITA and TRBC genes. In certain embodiments, a presently disclosed method comprises independently knocking out four T cell expressed genes, e.g., four selected from the group consisting of FAS, BID, CTLA4, PDCD1, CBLB, PTPN6, B2M, TRAC, CIITA and TRBC genes. In certain embodiments, a presently disclosed method comprises independently knocking out five T cell expressed genes, e.g., five selected from the group consisting of FAS, BID, CTLA4, PDCD1, CBLB, PTPN6, B2M, TRAC, CIITA and TRBC genes. In certain embodiments, a presently disclosed method comprises independently knocking out six T cell expressed genes, e.g., six selected from the group consisting of FAS, BID, CTLA4, PDCD1, CBLB, PTPN6, B2M, TRAC, CIITA and TRBC genes. In certain embodiments, a presently disclosed method comprises independently knocking out seven T cell expressed genes, e.g., seven selected from the group consisting of FAS, BID, CTLA4, PDCD1, CBLB, PTPN6, B2M, TRAC, CIITA and TRBC genes. In certain embodiments, a presently disclosed method comprises independently knocking out eight T cell expressed genes, e.g., selected from FAS, BID, CTLA4, PDCD1, CBLB, PTPN6, B2M, TRAC, CIITA and TRBC genes. In certain embodiments, a presently disclosed method comprises independently knocking out nine T cell expressed genes, e.g., selected from FAS, BID, CTLA4, PDCD1, CBLB, PTPN6, B2M, TRAC, CIITA and TRBC genes. In certain embodiments, a presently disclosed method comprises independently knocking out nine T cell expressed genes, e.g., FAS, BID, CTLA4, PDCD1, CBLB, PTPN6, B2M, TRAC, CIITA and TRBC genes.

In addition to the genes described above, a number of other T cell expressed genes may be targeted to affect the efficacy of engineered T cells. These genes include, but are not limited to, TGFBRI, TGFBRII and TGFBRIII (Kershaw et al. 2013 Nat. Rev. Cancer 13, 525-541). In certain embodiments, one or more of TGFBRI, TGFBRII and TGFBRIII genes can be modified either individually or in combination using the methods disclosed herein. In certain embodiments, one or more of TGFBRI, TGFBRII and TGFBRIII genes can be modified either individually or in combination with any one or more of the eight genes described above (i.e., FAS, BID, CTLA4, PDCD1, CBLB, PTPN6, B2M, TRAC, CIITA and TRBC genes) using the presently disclosed methods.

In certain embodiments, methods and compositions disclosed herein modify the FAS, BID, CTLA4, PDCD1, CBLB, PTPN6, B2M, TRAC, CIITA and/or TRBC genes by targeting a position (e.g., a knockout position) of the gene(s), e.g., a position within the non-coding region (e.g., the promoter region or a regulatory region) or a position within the coding region, or by targeting a transcribed sequence of the gene(s), e.g., an intronic sequence or an exonic sequence. In certain embodiments, a coding sequence, e.g., a coding region, e.g., an early coding region of the gene(s) (e.g., FAS, BID, CTLA4, PDCD1, CBLB, PTPN6, B2M, TRAC, CIITA and/or TRBC genes) is targeted for modification and knockout of expression. In certain embodiments, a position in the non-coding region (e.g., the promoter region or regulatory region) of the T cell expressed gene(s) (e.g., FAS, BID, CTLA4, PDCD1, CBLB, PTPN6, B2M, TRAC, CIITA and/or TRBC genes) is targeted for modification and knockout of expression of the T cell expressed gene(s).

In certain embodiments, the methods and compositions disclosed herein modify FAS, BID, CTLA4, PDCD1, CBLB, PTPN6, B2M, TRAC, CIITA and/or TRBC genes by targeting a coding sequence of the gene(s). In certain embodiments, the coding sequence is an early coding sequence. In certain embodiments, the coding sequence of the gene(s) is targeted for knockout of expression of the T cell expressed gene(s).

In certain embodiments, the methods and compositions disclosed herein modify FAS, BID, CTLA4, PDCD1, CBLB, PTPN6, B2M, TRAC, CIITA and/or TRBC genes by targeting a non-coding sequence of the gene(s). In certain embodiments, the non-coding sequence comprises a sequence within the promoter region, an enhancer sequence, an intronic sequence, a sequence within the 3′UTR, a polyadenylation signal sequence, or a combination thereof. In certain embodiments, the non-coding sequence of the gene(s) is targeted for knockout of expression of the gene(s).

In certain embodiments, a presently disclosed method comprises knocking out one or two alleles of FAS, BID, CTLA4, PDCD1, CBLB, PTPN6, B2M, TRAC, CIITA and/or TRBC gene(s), e.g., by inducing a modification in the gene(s). In certain embodiments, the modification comprises an insertion, a deletion, a mutation, or a combination thereof.

In certain embodiments, the targeted knockout approach is mediated by non-homologous end joining (NHEJ) using a CRISPR/Cpf1 system comprising a Cpf1 enzyme.

In certain embodiments, a CRISPR/Cpf1 system disclosed herein targets the TRAC gene. In certain embodiments, the CRISPR system comprises a gRNA complementary to a portion of the TRAC gene sequence. In certain embodiments, the gRNA can be complementary to either strand of the TRAC gene. In certain embodiments, the targeted portion of the TRAC gene sequence is within the coding sequence of the TRAC gene. In certain embodiments, the targeted portion of the TRAC gene sequence is within an exon. In certain embodiments, the targeted portion of the TRAC gene sequence is within an intron. In certain embodiments, the targeted portion of the TRAC gene sequence is within a regulatory region of the gene. In certain embodiments, more than one sequence is targeted and the targeted portions of the TRAC gene sequence are within one or more exons, one or more introns, one or more regulatory regions or one or more exons, one or more introns and one or more regulatory regions. In certain embodiments, the portion of the TRAC gene sequence is within the first 500 bp of the coding sequence of the TRAC gene. In certain embodiments, a targeting domain of a gRNA molecule for use in such a CRISPR/Cpf1 system targeting TRAC comprises a targeting domain sequence listed in Tables 2 and 3. The present disclosure provides compositions that include one or more of the gRNAs provided in Tables 2 and 3. The present disclosure further provides compositions that include one or more RNP complexes that include one or more of the gRNAs provided in Tables 2 and 3.

In certain embodiments, a CRISPR/Cpf1 system disclosed herein targets the TRBC gene. In certain embodiments, the CRISPR system comprises a gRNA complementary to a portion of the TRBC gene sequence. In certain embodiments, the gRNA can be complementary to either strand of the TRBC gene. In certain embodiments, the targeted portion of the TRBC gene sequence is within the coding sequence of the TRBC gene. In certain embodiments, the targeted portion of the TRBC gene sequence is within an exon. In certain embodiments, the targeted portion of the TRBC gene sequence is within an intron. In certain embodiments, the targeted portion of the TRBC gene sequence is within a regulatory region of the gene. In certain embodiments, more than one sequence is targeted and the targeted portions of the TRBC gene sequence are within one or more exons, one or more introns, one or more regulatory regions or one or more exons, one or more introns and one or more regulatory regions. In certain embodiments, the portion of the TRBC gene sequence is within the first 500 bp of the coding sequence of the TRBC gene. In certain embodiments, a targeting domain of a gRNA molecule for use in such a CRISPR/Cpf1 system targeting TRBC comprises a targeting domain sequence listed in Tables 4 and 5. The present disclosure provides compositions that include one or more of the gRNAs provided in Tables 4 and 5. The present disclosure further provides compositions that include one or more RNP complexes that include one or more of the gRNAs provided in Tables 4 and 5.

In certain embodiments, a CRISPR/Cpf1 system disclosed herein targets the B2M gene. In certain embodiments, the CRISPR system comprises a gRNA complementary to a portion of the B2M gene sequence. In certain embodiments, the gRNA can be complementary to either strand of the B2M gene. In certain embodiments, the targeted portion of the B2M gene sequence is within the coding sequence of the B2M gene. In certain embodiments, the targeted portion of the B2M gene sequence is within an exon. In certain embodiments, the targeted portion of the B2M gene sequence is within an intron. In certain embodiments, the targeted portion of the B2M gene sequence is within a regulatory region of the gene. In certain embodiments, more than one sequence is targeted and the targeted portions of the B2M gene sequence are within one or more exons, one or more introns, one or more regulatory regions or one or more exons, one or more introns and one or more regulatory regions. In certain embodiments, the portion of the B2M gene sequence is within the first 500 bp of the coding sequence of the B2M gene. In certain embodiments, the portion of the B2M gene sequence is between the 501st nucleotide and the last nucleotide of the coding sequence of the B2M gene. In certain embodiments, a targeting domain of a gRNA molecule for use in such a CRISPR/Cpf1 system targeting B2M comprises a targeting domain sequence listed in Tables 6, 7 and 8. In certain embodiments, a targeting domain of a gRNA molecule for use in such a CRISPR/Cpf1 system targeting B2M comprises AGUGGGGGUGAAUUCAGUGU. The present disclosure provides compositions that include one or more of the gRNAs provided in Tables 6, 7 and 8. The present disclosure further provides compositions that include one or more RNP complexes that include one or more of the gRNAs provided in Tables 6, 7 and 8.

In certain embodiments, a CRISPR/Cpf1 system disclosed herein targets the CIITA gene. In certain embodiments, the CRISPR system comprises a gRNA complementary to a portion of the CIITA gene sequence. In certain embodiments, the CRISPR system comprises a gRNA complementary to a portion of the CIITA gene sequence. In certain embodiments, the gRNA can be complementary to either strand of the CIITA gene. In certain embodiments, the targeted portion of the CIITA gene sequence is within the coding sequence of the CIITA gene. In certain embodiments, the targeted portion of the CIITA gene sequence is within an exon. In certain embodiments, the targeted portion of the CIITA gene sequence is within an intron. In certain embodiments, the targeted portion of the CIITA gene sequence is within a regulatory region of the gene. In certain embodiments, more than one sequence is targeted and the targeted portions of the CIITA gene sequence are within one or more exons, one or more introns, one or more regulatory regions or one or more exons, one or more introns and one or more regulatory regions. In certain embodiments, the portion of the CIITA gene sequence is within the first 500 bp of the coding sequence of the CIITA gene. In certain embodiments, a targeting domain of a gRNA molecule for use in such a CRISPR/Cpf1 system targeting CIITA comprises a targeting domain sequence listed in Table 9. The present disclosure provides compositions that include one or more of the gRNAs provided in Table 9. The present disclosure further provides compositions that include one or more RNP complexes that include one or more of the gRNAs provided in Table 9.

TABLE 2

| Gene | gRNA name | Targeting domain |
|---|---|---|
| TRAC | TRAC-1 AsCpf1 | UUGCUCCAGGCCACAGCACU |
| TRAC | TRAC-2 AsCpf1 | UCGACCAGCUUGACAUCACA |
| TRAC | TRAC-3 AsCpf1 | AGAAUCAAAAUCGGUGAAUA |
| TRAC | TRAC-4 AsCpf1 | CAUGUGCAAACGCCUUCAAC |
| TRAC | TRAC-5 AsCpf1 | AAAGUUUAGGUUCGUAUCUG |
| TRAC | TRAC-6 AsCpf1 | UUUGAGAAUCAAAAUCGGUG |
| TRAC | TRAC-7 AsCpf1 | AUUCUCAAACAAAUGUGUCA |
| TRAC | TRAC-8 AsCpf1 | CUUUUAGAAAGUUCCUGUGA |
| TRAC | TRAC-9 AsCpf1 | AAAGCUUUUCUCGACCAGCU |
| TRAC | TRAC-10 AsCpf1 | GAGUCUCUCAGCUGGUACAC |
| TRAC | TRAC-11 AsCpf1 | UCUGUGAUAUACACAUCAGA |
| TRAC | TRAC-12 AsCpf1 | UAAAAGGAAAAACAGACAUU |
| TRAC | TRAC-13 AsCpf1 | CAGAUACGAACCUAAACUUU |
| TRAC | TRAC-14 AsCpf1 | GAAAGUUCCUGUGAUGUCAA |
| TRAC | TRAC-15 AsCpf1 | GGUUCGUAUCUGUAAAACCA |
| TRAC | TRAC-16 AsCpf1 | AAAACCUGUCAGUGAUUGGG |
| TRAC | TRAC-17 AsCpf1 | AUCUGCUCAUGACGCUGCGG |
| TRAC | TRAC-18 AsCpf1 | CACAUGCAAAGUCAGAUUUG |
| TRAC | TRAC-19 AsCpf1 | UGACACAUUUGUUUGAGAAU |
| TRAC | TRAC-20 AsCpf1 | AAACAGGUAAGACAGGGGUC |
| TRAC | TRAC-21 AsCpf1 | AGGAGGAGGAUUCGGAACCC |
| TRAC | TRAC-1 AsCpf1 RR | GGCCACAGCACUGUUGCUCU |
| TRAC | TRAC-2 AsCpf1 RR | AGAAGACACCUUCUUCCCCA |
| TRAC | TRAC-3 AsCpf1 RR | UCACCUCAGCUGGACCACAG |
| TRAC | TRAC-4 AsCpf1 RR | ACAGAUAUCCAGAACCCUGA |
| TRAC | TRAC-5 AsCpf1 RR | UAUCUGUAAAACCAAGAGGC |
| TRAC | TRAC-6 AsCpf1 RR | AAUCCUCCUCCUGAAAGUGG |
| TRAC | TRAC-7 AsCpf1 RR | AGGCCCCUCACCUCAGCUGG |
| TRAC | TRAC-8 AsCpf1 RR | GAACCCAAUCACUGACAGGU |
| TRAC | TRAC-9 AsCpf1 RR | UGUGAUGUCAAGCUGGUCGA |
| TRAC | TRAC-10 AsCpf1 RR | ACUCCCAGCUUCAAGGCCCC |
| TRAC | TRAC-11 AsCpf1 RR | UGUCUUACCUGUUUCAAAGC |
| TRAC | TRAC-12 AsCpf1 RR | CAGCCCAGGUAAGGGCAGCU |
| TRAC | TRAC-13 AsCpf1 RR | GAACCCUGACCCUGCCGUGU |
| TRAC | TRAC-14 AsCpf1 RR | GAAUCCUCCUCCUGAAAGUG |
| TRAC | TRAC-15 AsCpf1 RR | GAAGACACCUUCUUCCCCAG |
| TRAC | TRAC-16 AsCpf1 RR | GGAGGAGGAUUCGGAACCCA |
| TRAC | TRAC-17 AsCpf1 RR | GCUGAGGUGAGGGGCCUUGA |
| TRAC | TRAC-18 AsCpf1 RR | GUGACAAGUCUGUCUGCCUA |
| TRAC | TRAC-19 AsCpf1 RR | AGCUUCAAGGCCCCUCACCU |
| TRAC | TRAC-20 AsCpf1 RR | AAGCUUUUCUCGACCAGCUU |
| TRAC | TRAC-21 AsCpf1 RR | CCAGCCCAGGUAAGGGCAGC |
| TRAC | TRAC-22 AsCpf1 RR | UUUUAGAAAGUUCCUGUGAU |
| TRAC | TRAC-23 AsCpf1 RR | AGCCCAGGUAAGGGCAGCUU |
| TRAC | TRAC-24 AsCpf1 RR | AGAGCAACAGUGCUGUGGCC |
| TRAC | TRAC-25 AsCpf1 RR | CCGAUUUUGAUUCUCAAACA |
| TRAC | TRAC-26 AsCpf1 RR | ACAACAGCAUUAUUCCAGAA |
| TRAC | TRAC-27 AsCpf1 RR | AAACCUGUCAGUGAUUGGGU |
| TRAC | TRAC-28 AsCpf1 RR | UAGACCUCAUGUCUAGCACA |
| TRAC | TRAC-1 AsCpf1 RVR | UCACAGACAAAACUGUGCUA |
| TRAC | TRAC-2 AsCpf1 RVR | CAGAACCCUGACCCUGCCGU |
| TRAC | TRAC-3 AsCpf1 RVR | GACUUCAAGAGCAACAGUGC |
| TRAC | TRAC-4 AsCpf1 RVR | UGUGGGACAAGAGGAUCAGG |
| TRAC | TRAC-5 AsCpf1 RVR | ACAGACAAAACUGUGCUAGA |
| TRAC | TRAC-6 AsCpf1 RVR | UGUAAAACCAAGAGGCCACA |
| TRAC | TRAC-7 AsCpf1 RVR | CACAUCAGAAUCCUUACUUU |

TABLE 3

| | | Within the first 500bp of coding | |
|---|---|---|---|
| | | sequence | |
| | | | Target |
| AsCpf1 | DNA | | Site |
| gRNA Name | Strand | Targeting Domain | Length |
| TRAC-c1 | - | AAAACCUGUCAGUGAUUG | 18 |
| TRAC-c2 | - | AAAACCUGUCAGUGAUUGG | 19 |
| TRAC-c3 | - | AAAACCUGUCAGUGAUUGGG | 20 |
| TRAC-c4 | - | AAAACCUGUCAGUGAUUGGGU | 21 |
| TRAC-c5 | - | AAAACCUGUCAGUGAUUGGGUU | 22 |
| TRAC-c6 | - | AAAACCUGUCAGUGAUUGGGUUC | 23 |
| TRAC-c7 | - | AAAACCUGUCAGUGAUUGGGUUCC | 24 |
| TRAC-c8 | - | AAACAGGUAAGACAGGGG | 18 |
| TRAC-c9 | - | AAACAGGUAAGACAGGGGU | 19 |
| TRAC-c10 | - | AAACAGGUAAGACAGGGGUC | 20 |
| TRAC-c11 | - | AAACAGGUAAGACAGGGGUCU | 21 |
| TRAC-c12 | - | AAACAGGUAAGACAGGGGUCUA | 22 |
| TRAC-c13 | - | AAACAGGUAAGACAGGGGUCUAG | 23 |
| TRAC-c14 | - | AAACAGGUAAGACAGGGGUCUAGC | 24 |
| TRAC-c15 | + | AAAGCUUUUCUCGACCAG | 18 |
| TRAC-c16 | + | AAAGCUUUUCUCGACCAGC | 19 |
| TRAC-c17 | + | AAAGCUUUUCUCGACCAGCU | 20 |
| TRAC-c18 | + | AAAGCUUUUCUCGACCAGCUU | 21 |
| TRAC-c19 | + | AAAGCUUUUCUCGACCAGCUUG | 22 |
| TRAC-c20 | + | AAAGCUUUUCUCGACCAGCUUGA | 23 |
| TRAC-c21 | + | AAAGCUUUUCUCGACCAGCUUGAC | 24 |
| TRAC-c22 | + | AAAGUUUAGGUUCGUAUC | 18 |
| TRAC-c23 | + | AAAGUUUAGGUUCGUAUCU | 19 |
| TRAC-c24 | + | AAAGUUUAGGUUCGUAUCUG | 20 |
| TRAC-c25 | + | AAAGUUUAGGUUCGUAUCUGU | 21 |
| TRAC-c26 | + | AAAGUUUAGGUUCGUAUCUGUA | 22 |
| TRAC-c27 | + | AAAGUUUAGGUUCGUAUCUGUAA | 23 |
| TRAC-c28 | + | AAAGUUUAGGUUCGUAUCUGUAAA | 24 |
| TRAC-c29 | - | ACAGAUACGAACCUAAAC | 18 |
| TRAC-c30 | - | ACAGAUACGAACCUAAACU | 19 |
| TRAC-c31 | - | ACAGAUACGAACCUAAACUU | 20 |
| TRAC-c32 | - | ACAGAUACGAACCUAAACUUU | 21 |
| TRAC-c33 | - | ACAGAUACGAACCUAAACUUUC | 22 |
| TRAC-c34 | - | ACAGAUACGAACCUAAACUUUCA | 23 |
| TRAC-c35 | - | ACAGAUACGAACCUAAACUUUCAA | 24 |
| TRAC-c36 | - | AGAAAGUUCCUGUGAUGU | 18 |
| TRAC-c37 | - | AGAAAGUUCCUGUGAUGUC | 19 |
| TRAC-c38 | - | AGAAAGUUCCUGUGAUGUCA | 20 |
| TRAC-c39 | - | AGAAAGUUCCUGUGAUGUCAA | 21 |
| TRAC-c40 | - | AGAAAGUUCCUGUGAUGUCAAG | 22 |
| TRAC-c41 | - | AGAAAGUUCCUGUGAUGUCAAGC | 23 |
| TRAC-c42 | - | AGAAAGUUCCUGUGAUGUCAAGCU | 24 |
| TRAC-c43 | + | AGAAUCAAAAUCGGUGAA | 18 |
| TRAC-c44 | + | AGAAUCAAAAUCGGUGAAU | 19 |
| TRAC-c45 | + | AGAAUCAAAAUCGGUGAAUA | 20 |
| TRAC-c46 | + | AGAAUCAAAAUCGGUGAAUAG | 21 |
| TRAC-c47 | + | AGAAUCAAAAUCGGUGAAUAGG | 22 |
| TRAC-c48 | + | AGAAUCAAAAUCGGUGAAUAGGC | 23 |
| TRAC-c49 | + | AGAAUCAAAAUCGGUGAAUAGGCA | 24 |
| TRAC-c50 | + | AGGAGGAGGAUUCGGAAC | 18 |
| TRAC-c51 | + | AGGAGGAGGAUUCGGAACC | 19 |
| TRAC-c52 | + | AGGAGGAGGAUUCGGAACCC | 20 |
| TRAC-c53 | + | AGGAGGAGGAUUCGGAACCCA | 21 |
| TRAC-c54 | + | AGGAGGAGGAUUCGGAACCCAA | 22 |
| TRAC-c55 | + | AGGAGGAGGAUUCGGAACCCAAU | 23 |
| TRAC-c56 | + | AGGAGGAGGAUUCGGAACCCAAUC | 24 |
| TRAC-c57 | - | AUCUGCUCAUGACGCUGC | 18 |
| TRAC-c58 | - | AUCUGCUCAUGACGCUGCG | 19 |
| TRAC-c59 | - | AUCUGCUCAUGACGCUGCGG | 20 |
| TRAC-c60 | - | AUCUGCUCAUGACGCUGCGGC | 21 |
| TRAC-c61 | - | AUCUGCUCAUGACGCUGCGGCU | 22 |
| TRAC-c62 | - | AUCUGCUCAUGACGCUGCGGCUG | 23 |
| TRAC-c63 | - | AUCUGCUCAUGACGCUGCGGCUGU | 24 |
| TRAC-c64 | - | AUUCUCAAACAAAUGUGU | 18 |
| TRAC-c65 | - | AUUCUCAAACAAAUGUGUC | 19 |
| TRAC-c66 | - | AUUCUCAAACAAAUGUGUCA | 20 |
| TRAC-c67 | - | AUUCUCAAACAAAUGUGUCAC | 21 |
| TRAC-c68 | - | AUUCUCAAACAAAUGUGUCACA | 22 |
| TRAC-c69 | - | AUUCUCAAACAAAUGUGUCACAA | 23 |
| TRAC-c70 | - | AUUCUCAAACAAAUGUGUCACAAA | 24 |
| TRAC-c71 | + | CACAUGCAAAGUCAGAUU | 18 |
| TRAC-c72 | + | CACAUGCAAAGUCAGAUUU | 19 |
| TRAC-c73 | + | CACAUGCAAAGUCAGAUUUG | 20 |
| TRAC-c74 | + | CACAUGCAAAGUCAGAUUUGU | 21 |
| TRAC-c75 | + | CACAUGCAAAGUCAGAUUUGUU | 22 |
| TRAC-c76 | + | CACAUGCAAAGUCAGAUUUGUUG | 23 |
| TRAC-c77 | + | CACAUGCAAAGUCAGAUUUGUUGC | 24 |
| TRAC-c78 | - | CAGAUACGAACCUAAACU | 18 |
| TRAC-c79 | - | CAGAUACGAACCUAAACUU | 19 |
| TRAC-c80 | - | CAGAUACGAACCUAAACUUU | 20 |
| TRAC-c81 | - | CAGAUACGAACCUAAACUUUC | 21 |
| TRAC-c82 | - | CAGAUACGAACCUAAACUUUCA | 22 |
| TRAC-c83 | - | CAGAUACGAACCUAAACUUUCAA | 23 |
| TRAC-c84 | - | CAGAUACGAACCUAAACUUUCAAA | 24 |
| TRAC-c85 | - | CAUGUGCAAACGCCUUCA | 18 |
| TRAC-c86 | - | CAUGUGCAAACGCCUUCAA | 19 |
| TRAC-c87 | - | CAUGUGCAAACGCCUUCAAC | 20 |
| TRAC-c88 | - | CAUGUGCAAACGCCUUCAACA | 21 |
| TRAC-c89 | - | CAUGUGCAAACGCCUUCAACAA | 22 |
| TRAC-c90 | - | CAUGUGCAAACGCCUUCAACAAC | 23 |
| TRAC-c91 | - | CAUGUGCAAACGCCUUCAACAACA | 24 |
| TRAC-c92 | - | CCUUUUAGAAAGUUCCUG | 18 |
| TRAC-c93 | - | CCUUUUAGAAAGUUCCUGU | 19 |
| TRAC-c94 | - | CCUUUUAGAAAGUUCCUGUG | 20 |
| TRAC-c95 | - | CCUUUUAGAAAGUUCCUGUGA | 21 |
| TRAC-c96 | - | CCUUUUAGAAAGUUCCUGUGAU | 22 |
| TRAC-c97 | - | CCUUUUAGAAAGUUCCUGUGAUG | 23 |
| TRAC-c98 | - | CCUUUUAGAAAGUUCCUGUGAUGU | 24 |
| TRAC-c99 | + | CUCGACCAGCUUGACAUC | 18 |
| TRAC-c100 | + | CUCGACCAGCUUGACAUCA | 19 |
| TRAC-c101 | + | CUCGACCAGCUUGACAUCAC | 20 |
| TRAC-c102 | + | CUCGACCAGCUUGACAUCACA | 21 |
| TRAC-c103 | + | CUCGACCAGCUUGACAUCACAG | 22 |
| TRAC-c104 | + | CUCGACCAGCUUGACAUCACAGG | 23 |
| TRAC-c105 | + | CUCGACCAGCUUGACAUCACAGGA | 24 |
| TRAC-c106 | - | CUUUUAGAAAGUUCCUGU | 18 |
| TRAC-c107 | - | CUUUUAGAAAGUUCCUGUG | 19 |
| TRAC-c108 | - | CUUUUAGAAAGUUCCUGUGA | 20 |
| TRAC-c109 | - | CUUUUAGAAAGUUCCUGUGAU | 21 |
| TRAC-c110 | - | CUUUUAGAAAGUUCCUGUGAUG | 22 |
| TRAC-c111 | - | CUUUUAGAAAGUUCCUGUGAUGU | 23 |
| TRAC-c112 | - | CUUUUAGAAAGUUCCUGUGAUGUC | 24 |
| TRAC-c113 | - | GAAAGUUCCUGUGAUGUC | 18 |
| TRAC-c114 | - | GAAAGUUCCUGUGAUGUCA | 19 |
| TRAC-c115 | - | GAAAGUUCCUGUGAUGUCAA | 20 |
| TRAC-c116 | - | GAAAGUUCCUGUGAUGUCAAG | 21 |
| TRAC-c117 | - | GAAAGUUCCUGUGAUGUCAAGC | 22 |
| TRAC-c118 | - | GAAAGUUCCUGUGAUGUCAAGCU | 23 |
| TRAC-c119 | - | GAAAGUUCCUGUGAUGUCAAGCUG | 24 |
| TRAC-c120 | + | GAAAGUUUAGGUUCGUAU | 18 |
| TRAC-c121 | + | GAAAGUUUAGGUUCGUAUC | 19 |
| TRAC-c122 | + | GAAAGUUUAGGUUCGUAUCU | 20 |
| TRAC-c123 | + | GAAAGUUUAGGUUCGUAUCUG | 21 |
| TRAC-c124 | + | GAAAGUUUAGGUUCGUAUCUGU | 22 |
| TRAC-c125 | + | GAAAGUUUAGGUUCGUAUCUGUA | 23 |
| TRAC-c126 | + | GAAAGUUUAGGUUCGUAUCUGUAA | 24 |
| TRAC-c127 | + | GAGUCUCUCAGCUGGUAC | 18 |
| TRAC-c128 | + | GAGUCUCUCAGCUGGUACA | 19 |
| TRAC-c129 | + | GAGUCUCUCAGCUGGUACAC | 20 |
| TRAC-c130 | + | GAGUCUCUCAGCUGGUACACG | 21 |
| TRAC-c131 | + | GAGUCUCUCAGCUGGUACACGG | 22 |
| TRAC-c132 | + | GAGUCUCUCAGCUGGUACACGGC | 23 |
| TRAC-c133 | + | GAGUCUCUCAGCUGGUACACGGCA | 24 |
| TRAC-c134 | - | GAUUCUCAAACAAAUGUG | 18 |
| TRAC-c135 | - | GAUUCUCAAACAAAUGUGU | 19 |
| TRAC-c136 | - | GAUUCUCAAACAAAUGUGUC | 20 |
| TRAC-c137 | - | GAUUCUCAAACAAAUGUGUCA | 21 |
| TRAC-c138 | - | GAUUCUCAAACAAAUGUGUCAC | 22 |
| TRAC-c139 | - | GAUUCUCAAACAAAUGUGUCACA | 23 |
| TRAC-c140 | - | GAUUCUCAAACAAAUGUGUCACAA | 24 |
| TRAC-c141 | + | GGUUCGUAUCUGUAAAAC | 18 |
| TRAC-c142 | + | GGUUCGUAUCUGUAAAACC | 19 |
| TRAC-c143 | + | GGUUCGUAUCUGUAAAACCA | 20 |
| TRAC-c144 | + | GGUUCGUAUCUGUAAAACCAA | 21 |
| TRAC-c145 | + | GGUUCGUAUCUGUAAAACCAAG | 22 |
| TRAC-c146 | + | GGUUCGUAUCUGUAAAACCAAGA | 23 |
| TRAC-c147 | + | GGUUCGUAUCUGUAAAACCAAGAG | 24 |
| TRAC-c148 | + | GUCUGUGAUAUACACAUC | 18 |
| TRAC-c149 | + | GUCUGUGAUAUACACAUCA | 19 |
| TRAC-c150 | + | GUCUGUGAUAUACACAUCAG | 20 |
| TRAC-c151 | + | GUCUGUGAUAUACACAUCAGA | 21 |
| TRAC-c152 | + | GUCUGUGAUAUACACAUCAGAA | 22 |
| TRAC-c153 | + | GUCUGUGAUAUACACAUCAGAAU | 23 |
| TRAC-c154 | + | GUCUGUGAUAUACACAUCAGAAUC | 24 |
| TRAC-c155 | + | UAAAAGGAAAAACAGACA | 18 |
| TRAC-c156 | + | UAAAAGGAAAAACAGACAU | 19 |
| TRAC-c157 | + | UAAAAGGAAAAACAGACAUU | 20 |
| TRAC-c158 | + | UAAAAGGAAAAACAGACAUUC | 21 |
| TRAC-c159 | + | UAAAAGGAAAAACAGACAUUCU | 22 |
| TRAC-c160 | + | UAAAAGGAAAAACAGACAUUCUU | 23 |
| TRAC-c161 | + | UAAAAGGAAAAACAGACAUUCUUU | 24 |
| TRAC-c162 | - | UCCUUUUAGAAAGUUCCU | 18 |
| TRAC-c163 | - | UCCUUUUAGAAAGUUCCUG | 19 |
| TRAC-c164 | - | UCCUUUUAGAAAGUUCCUGU | 20 |
| TRAC-c165 | - | UCCUUUUAGAAAGUUCCUGUG | 21 |
| TRAC-c166 | - | UCCUUUUAGAAAGUUCCUGUGA | 22 |
| TRAC-c167 | - | UCCUUUUAGAAAGUUCCUGUGAU | 23 |
| TRAC-c168 | - | UCCUUUUAGAAAGUUCCUGUGAUG | 24 |
| TRAC-c169 | + | UCGACCAGCUUGACAUCA | 18 |
| TRAC-c170 | + | UCGACCAGCUUGACAUCAC | 19 |
| TRAC-c171 | + | UCGACCAGCUUGACAUCACA | 20 |
| TRAC-c172 | + | UCGACCAGCUUGACAUCACAG | 21 |
| TRAC-c173 | + | UCGACCAGCUUGACAUCACAGG | 22 |
| TRAC-c174 | + | UCGACCAGCUUGACAUCACAGGA | 23 |
| TRAC-c175 | + | UCGACCAGCUUGACAUCACAGGAA | 24 |
| TRAC-c176 | + | UCUGUGAUAUACACAUCA | 18 |
| TRAC-c177 | + | UCUGUGAUAUACACAUCAG | 19 |
| TRAC-c178 | + | UCUGUGAUAUACACAUCAGA | 20 |
| TRAC-c179 | + | UCUGUGAUAUACACAUCAGAA | 21 |
| TRAC-c180 | + | UCUGUGAUAUACACAUCAGAAU | 22 |
| TRAC-c181 | + | UCUGUGAUAUACACAUCAGAAUC | 23 |
| TRAC-c182 | + | UCUGUGAUAUACACAUCAGAAUCC | 24 |
| TRAC-c183 | + | UGACACAUUUGUUUGAGA | 18 |
| TRAC-c184 | + | UGACACAUUUGUUUGAGAA | 19 |
| TRAC-c185 | + | UGACACAUUUGUUUGAGAAU | 20 |
| TRAC-c186 | + | UGACACAUUUGUUUGAGAAUC | 21 |
| TRAC-c187 | + | UGACACAUUUGUUUGAGAAUCA | 22 |
| TRAC-c188 | + | UGACACAUUUGUUUGAGAAUCAA | 23 |
| TRAC-c189 | + | UGACACAUUUGUUUGAGAAUCAAA | 24 |
| TRAC-c190 | + | UUGCUCCAGGCCACAGCA | 18 |
| TRAC-c191 | + | UUGCUCCAGGCCACAGCAC | 19 |
| TRAC-c192 | + | UUGCUCCAGGCCACAGCACU | 20 |
| TRAC-c193 | + | UUGCUCCAGGCCACAGCACUG | 21 |
| TRAC-c194 | + | UUGCUCCAGGCCACAGCACUGU | 22 |
| TRAC-c195 | + | UUGCUCCAGGCCACAGCACUGUU | 23 |
| TRAC-c196 | + | UUGCUCCAGGCCACAGCACUGUUG | 24 |
| TRAC-c197 | + | UUUGAGAAUCAAAAUCGG | 18 |
| TRAC-c198 | + | UUUGAGAAUCAAAAUCGGU | 19 |
| TRAC-c199 | + | UUUGAGAAUCAAAAUCGGUG | 20 |
| TRAC-c200 | + | UUUGAGAAUCAAAAUCGGUGA | 21 |
| TRAC-c201 | + | UUUGAGAAUCAAAAUCGGUGAA | 22 |
| TRAC-c202 | + | UUUGAGAAUCAAAAUCGGUGAAU | 23 |
| TRAC-c203 | + | UUUGAGAAUCAAAAUCGGUGAAUA | 24 |

TABLE 4

| Gene | gRNA name | Targeting domain |
|---|---|---|
| TRBC | TRBC-1 AsCpf1 | CAGAGGACCUGAAAAACGUG |
| TRBC | TRBC-2 AsCpf1 | AGGUCCUCUGGAAAGGGAAG |
| TRBC | TRBC-3 AsCpf1 | AGCCAUCAGAAGCAGAGAUC |
| TRBC | TRBC-4 AsCpf1 | GGUGUGGGAGAUCUCUGCUU |
| TRBC | TRBC-5 AsCpf1 | GCCCUAUCCUGGGUCCACUC |
| TRBC | TRBC-6 AsCpf1 | UUCCCCUGUUUUCUUUCAGA |
| TRBC | TRBC-7 AsCpf1 | UUUCAGACUGUGGCUUCACC |
| TRBC | TRBC-1 AsCpf1 RR | AGGCCUCGGCGCUGACGAUC |
| TRBC | TRBC-2 AsCpf1 RR | CAGGCCCCACUCACCUGCUC |
| TRBC | TRBC-3 AsCpf1 RR | AGGCCCCACUCACCUGCUCU |
| TRBC | TRBC-4 AsCpf1 RR | ACUCACCUGCUCUACCCCAG |
| TRBC | TRBC-5 AsCpf1 RR | AGAGCCCGUAGAACUGGACU |
| TRBC | TRBC-6 AsCpf1 RR | CUCGUCAUUCUCCGAGAGCC |
| TRBC | TRBC-7 AsCpf1 RR | GCAACCACUUCCGCUGUCAA |
| TRBC | TRBC-8 AsCpf1 RR | GCUGUCAAGUCCAGUUCUAC |
| TRBC | TRBC-9 AsCpf1 RR | CUGUCAAGUCCAGUUCUACG |
| TRBC | TRBC-10 AsCpf1 RR | GUUCUACGGGCUCUCGGAGA |
| TRBC | TRBC-11 AsCpf1 RR | CUUUCCAGAGGACCUGAAAA |
| TRBC | TRBC-12 AsCpf1 RR | UUUCCAGAGGACCUGAAAAA |
| TRBC | TRBC-13 AsCpf1 RR | AGAGGACCUGAAAAACGUGU |
| TRBC | TRBC-14 AsCpf1 RR | GGUCCUCUGGAAAGGGAAGA |
| TRBC | TRBC-15 AsCpf1 RR | GAGGACCUGAAAAACGUGUU |
| TRBC | TRBC-16 AsCpf1 RR | CACCCGAGGUCGCUGUGUUU |
| TRBC | TRBC-17 AsCpf1 RR | ACACCCAAAAGGCCACACUG |
| TRBC | TRBC-18 AsCpf1 RR | GACCACGUGGAGCUGAGCUG |
| TRBC | TRBC-19 AsCpf1 RR | CGUGGUCGGGGUAGAAGCCU |
| TRBC | TRBC-20 AsCpf1 RR | CCCACCAGCUCAGCUCCACG |
| TRBC | TRBC-21 AsCpf1 RR | AUUCACCCACCAGCUCAGCU |
| TRBC | TRBC-22 AsCpf1 RR | CAUUCACCCACCAGCUCAGC |
| TRBC | TRBC-23 AsCpf1 RR | ACUGUGCACCUCCUUCCCAU |
| TRBC | TRBC-24 AsCpf1 RR | UCAAGGAGCAGCCCGCCCUC |
| TRBC | TRBC-25 AsCpf1 RR | GAUACUGCCUGAGCAGCCGC |
| TRBC | TRBC-26 AsCpf1 RR | CGCAACCACUUCCGCUGUCA |
| TRBC | TRBC-27 AsCpf1 RR | GGCAUCUCCCCAGGCCCCAC |
| TRBC | TRBC-28 AsCpf1 RR | CUGUUUUCUUUCAGACUGUG |
| TRBC | TRBC-29 AsCpf1 RR | GACUGUGGCUUCACCUCCGG |
| TRBC | TRBC-30 AsCpf1 RR | CCUCCGGUAAGUGAGUCUCU |
| TRBC | TRBC-31 AsCpf1 RR | UAGCAAGAUCUCAUAGAGGA |
| TRBC | TRBC-32 AsCpf1 RR | CUAGCAAGAUCUCAUAGAGG |
| TRBC | TRBC-33 AsCpf1 RR | ACCCUCCUCCUUACCAUGGC |
| TRBC | TRBC-34 AsCpf1 RR | AUUACCUCUUCCCUUUCCAG |
| TRBC | TRBC-1 AsCpf1 RVR | UGGAGUCAUUGAGGGCGGGC |
| TRBC | TRBC-2 AsCpf1 RVR | CUGGGUCCACUCGUCAUUCU |
| TRBC | TRBC-3 AsCpf1 RVR | AGAUCUUGCUAGGGAAGGCC |
| TRBC | TRBC-4 AsCpf1 RVR | CCGUGCUGGUCAGUGCCCUC |
| TRBC | TRBC-5 AsCpf1 RVR | CCACCCUCCUCCUUACCAUG |

TABLE 5

| | Within the first 500 bp of coding | | |
|---|---|---|---|
| | sequence | | |
| AsCpf1 | | | Target |
| gRNA | DNA | | Site |
| Name | Strand | Targeting Domain | Length |
| TRBC-c1 | − | AGACUGUGGCUUCACCUC | 18 |
| TRBC-c2 | − | AGACUGUGGCUUCACCUCC | 19 |
| TRBC-c3 | − | AGACUGUGGCUUCACCUCCG | 20 |
| TRBC-c4 | − | AGACUGUGGCUUCACCUCCGG | 21 |
| TRBC-c5 | − | AGACUGUGGCUUCACCUCCGGU | 22 |
| TRBC-c6 | − | AGACUGUGGCUUCACCUCCGGUA | 23 |
| TRBC-c7 | − | AGACUGUGGCUUCACCUCCGGUAA | 24 |
| TRBC-c8 | − | AGCCAUCAGAAGCAGAGA | 18 |
| TRBC-c9 | − | AGCCAUCAGAAGCAGAGAU | 19 |
| TRBC-c10 | − | AGCCAUCAGAAGCAGAGAUC | 20 |
| TRBC-c11 | − | AGCCAUCAGAAGCAGAGAUCU | 21 |
| TRBC-c12 | − | AGCCAUCAGAAGCAGAGAUCUC | 22 |
| TRBC-c13 | − | AGCCAUCAGAAGCAGAGAUCUCC | 23 |
| TRBC-c14 | − | AGCCAUCAGAAGCAGAGAUCUCCC | 24 |
| TRBC-c15 | + | AGGUCCUCUGGAAAGGGA | 18 |
| TRBC-c16 | + | AGGUCCUCUGGAAAGGGAA | 19 |
| TRBC-c17 | + | AGGUCCUCUGGAAAGGGAAG | 20 |
| TRBC-c18 | + | AGGUCCUCUGGAAAGGGAAGA | 21 |
| TRBC-c19 | + | AGGUCCUCUGGAAAGGGAAGAG | 22 |
| TRBC-c20 | + | AGGUCCUCUGGAAAGGGAAGAGG | 23 |
| TRBC-c21 | + | AGGUCCUCUGGAAAGGGAAGAGGU | 24 |
| TRBC-c22 | − | CAGAGGACCUGAAAAACG | 18 |
| TRBC-c23 | − | CAGAGGACCUGAAAAACGU | 19 |
| TRBC-c24 | − | CAGAGGACCUGAAAAACGUG | 20 |
| TRBC-c25 | − | CAGAGGACCUGAAAAACGUGU | 21 |
| TRBC-c26 | − | CAGAGGACCUGAAAAACGUGUU | 22 |
| TRBC-c27 | − | CAGAGGACCUGAAAAACGUGUUC | 23 |
| TRBC-c28 | − | CAGAGGACCUGAAAAACGUGUUCC | 24 |
| TRBC-c29 | + | CAGGUCCUCUGGAAAGGG | 18 |
| TRBC-c30 | + | CAGGUCCUCUGGAAAGGGA | 19 |
| TRBC-c31 | + | CAGGUCCUCUGGAAAGGGAA | 20 |
| TRBC-c32 | + | CAGGUCCUCUGGAAAGGGAAG | 21 |
| TRBC-c33 | + | CAGGUCCUCUGGAAAGGGAAGA | 22 |
| TRBC-c34 | + | CAGGUCCUCUGGAAAGGGAAGAG | 23 |
| TRBC-c35 | + | CAGGUCCUCUGGAAAGGGAAGAGG | 24 |
| TRBC-c36 | − | CUUCCCCUGUUUUCUUUC | 18 |
| TRBC-c37 | − | CUUCCCCUGUUUUCUUUCA | 19 |
| TRBC-c38 | − | CUUCCCCUGUUUUCUUUCAG | 20 |
| TRBC-c39 | − | CUUCCCCUGUUUUCUUUCAGA | 21 |
| TRBC-c40 | − | CUUCCCCUGUUUUCUUUCAGAC | 22 |
| TRBC-c41 | − | CUUCCCCUGUUUUCUUUCAGACU | 23 |
| TRBC-c42 | − | CUUCCCCUGUUUUCUUUCAGACUG | 24 |
| TRBC-c43 | − | CUUUCAGACUGUGGCUUC | 18 |
| TRBC-c44 | − | CUUUCAGACUGUGGCUUCA | 19 |
| TRBC-c45 | − | CUUUCAGACUGUGGCUUCAC | 20 |
| TRBC-c46 | − | CUUUCAGACUGUGGCUUCACC | 21 |
| TRBC-c47 | − | CUUUCAGACUGUGGCUUCACCU | 22 |
| TRBC-c48 | − | CUUUCAGACUGUGGCUUCACCUC | 23 |
| TRBC-c49 | − | CUUUCAGACUGUGGCUUCACCUCC | 24 |
| TRBC-c50 | + | GAGCUAGCCUCUGGAAUC | 18 |
| TRBC-c51 | + | GAGCUAGCCUCUGGAAUCC | 19 |
| TRBC-c52 | + | GAGCUAGCCUCUGGAAUCCU | 20 |
| TRBC-c53 | + | GAGCUAGCCUCUGGAAUCCUU | 21 |
| TRBC-c54 | + | GAGCUAGCCUCUGGAAUCCUUU | 22 |
| TRBC-c55 | + | GAGCUAGCCUCUGGAAUCCUUUC | 23 |
| TRBC-c56 | + | GAGCUAGCCUCUGGAAUCCUUUCU | 24 |
| TRBC-c57 | + | GCCCUAUCCUGGGUCCAC | 18 |
| TRBC-c58 | + | GCCCUAUCCUGGGUCCACU | 19 |
| TRBC-c59 | + | GCCCUAUCCUGGGUCCACUC | 20 |
| TRBC-c60 | + | GCCCUAUCCUGGGUCCACUCG | 21 |
| TRBC-c61 | + | GCCCUAUCCUGGGUCCACUCGU | 22 |
| TRBC-c62 | + | GCCCUAUCCUGGGUCCACUCGUC | 23 |
| TRBC-c63 | + | GCCCUAUCCUGGGUCCACUCGUCA | 24 |
| TRBC-c64 | + | GGAGCUAGCCUCUGGAAU | 18 |
| TRBC-c65 | + | GGAGCUAGCCUCUGGAAUC | 19 |
| TRBC-c66 | + | GGAGCUAGCCUCUGGAAUCC | 20 |
| TRBC-c67 | + | GGAGCUAGCCUCUGGAAUCCU | 21 |
| TRBC-c68 | + | GGAGCUAGCCUCUGGAAUCCUU | 22 |
| TRBC-c69 | + | GGAGCUAGCCUCUGGAAUCCUUU | 23 |
| TRBC-c70 | + | GGAGCUAGCCUCUGGAAUCCUUUC | 24 |
| TRBC-c71 | + | GGGUGUGGGAGAUCUCUG | 18 |
| TRBC-c72 | + | GGGUGUGGGAGAUCUCUGC | 19 |
| TRBC-c73 | + | GGGUGUGGGAGAUCUCUGCU | 20 |
| TRBC-c74 | + | GGGUGUGGGAGAUCUCUGCUU | 21 |
| TRBC-c75 | + | GGGUGUGGGAGAUCUCUGCUUC | 22 |
| TRBC-c76 | + | GGGUGUGGGAGAUCUCUGCUUCU | 23 |
| TRBC-c77 | + | GGGUGUGGGAGAUCUCUGCUUCUG | 24 |
| TRBC-c78 | + | GGUGUGGGAGAUCUCUGC | 18 |
| TRBC-c79 | + | GGUGUGGGAGAUCUCUGCU | 19 |
| TRBC-c80 | + | GGUGUGGGAGAUCUCUGCUU | 20 |
| TRBC-c81 | + | GGUGUGGGAGAUCUCUGCUUC | 21 |
| TRBC-c82 | + | GGUGUGGGAGAUCUCUGCUUCU | 22 |
| TRBC-c83 | + | GGUGUGGGAGAUCUCUGCUUCUG | 23 |
| TRBC-c84 | + | GGUGUGGGAGAUCUCUGCUUCUGA | 24 |
| TRBC-c85 | + | UCAGGUCCUCUGGAAAGG | 18 |
| TRBC-c86 | + | UCAGGUCCUCUGGAAAGGG | 19 |
| TRBC-c87 | + | UCAGGUCCUCUGGAAAGGGA | 20 |
| TRBC-c88 | + | UCAGGUCCUCUGGAAAGGGAA | 21 |
| TRBC-c89 | + | UCAGGUCCUCUGGAAAGGGAAG | 22 |
| TRBC-c90 | + | UCAGGUCCUCUGGAAAGGGAAGA | 23 |
| TRBC-c91 | + | UCAGGUCCUCUGGAAAGGGAAGAG | 24 |
| TRBC-c92 | − | UCUUCCCCUGUUUUCUUU | 18 |
| TRBC-c93 | − | UCUUCCCCUGUUUUCUUUC | 19 |
| TRBC-c94 | − | UCUUCCCCUGUUUUCUUUCA | 20 |
| TRBC-c95 | − | UCUUCCCCUGUUUUCUUUCAG | 21 |
| TRBC-c96 | − | UCUUCCCCUGUUUUCUUUCAGA | 22 |
| TRBC-c97 | − | UCUUCCCCUGUUUUCUUUCAGAC | 23 |
| TRBC-c98 | − | UCUUCCCCUGUUUUCUUUCAGACU | 24 |
| TRBC-c99 | + | UCUUGACCUGUGGAAGAG | 18 |
| TRBC-c100 | + | UCUUGACCUGUGGAAGAGA | 19 |
| TRBC-c101 | + | UCUUGACCUGUGGAAGAGAG | 20 |
| TRBC-c102 | + | UCUUGACCUGUGGAAGAGAGA | 21 |
| TRBC-c103 | + | UCUUGACCUGUGGAAGAGAGAA | 22 |
| TRBC-c104 | + | UCUUGACCUGUGGAAGAGAGAAC | 23 |
| TRBC-c105 | + | UCUUGACCUGUGGAAGAGAGAACA | 24 |
| TRBC-c106 | − | UUCCCCUGUUUUCUUUCA | 18 |
| TRBC-c107 | − | UUCCCCUGUUUUCUUUCAG | 19 |
| TRBC-c108 | − | UUCCCCUGUUUUCUUUCAGA | 20 |
| TRBC-c109 | − | UUCCCCUGUUUUCUUUCAGAC | 21 |
| TRBC-c110 | − | UUCCCCUGUUUUCUUUCAGACU | 22 |
| TRBC-c111 | − | UUCCCCUGUUUUCUUUCAGACUG | 23 |
| TRBC-c112 | − | UUCCCCUGUUUUCUUUCAGACUGU | 24 |
| TRBC-c113 | − | UUUCAGACUGUGGCUUCA | 18 |
| TRBC-c114 | − | UUUCAGACUGUGGCUUCAC | 19 |
| TRBC-c115 | − | UUUCAGACUGUGGCUUCACC | 20 |
| TRBC-c116 | − | UUUCAGACUGUGGCUUCACCU | 21 |
| TRBC-c117 | − | UUUCAGACUGUGGCUUCACCUC | 22 |
| TRBC-c118 | − | UUUCAGACUGUGGCUUCACCUCC | 23 |
| TRBC-c119 | − | UUUCAGACUGUGGCUUCACCUCCG | 24 |

TABLE 6

| Gene | gRNA Name | Targeting Domain |
|---|---|---|
| B2M | B2M-1 AsCpf1 | UGGCCUGGAGGCUAUCCAGC |
| B2M | B2M-2 AsCp1 | CCGAUAUUCCUCAGGUACUC |
| B2M | B2M-3 AsCpf1 | GAGUACCUGAGGAAUAUCGG |
| B2M | B2M-4 AsCpf1 | CUCACGUCAUCCAGCAGAGA |
| B2M | B2M-5 AsCpf1 | CAUUCUCUGCUGGAUGACGU |
| B2M | B2M-6 AsCpf1 | ACUUUCCAUUCUCUGCUGGA |
| B2M | B2M-7 AsCpf1 | CUGAAUUGCUAUGUGUCUGG |
| B2M | B2M-8 AsCpf1 | AUCCAUCCGACAUUGAAGUU |
| B2M | B2M-9 AsCpf1 | AAUUCUCUCUCCAUUCUUCA |
| B2M | B2M-10 AsCpf1 | AGCAAGGACUGGUCUUUCUA |
| B2M | B2M-11 AsCpf1 | UAUCUCUUGUACUACACUGA |
| B2M | B2M-12 AsCpf1 | AGUGGGGGUGAAUUCAGUGU |
| B2M | B2M-13 AsCpf1 | UCACAGCCCAAGAUAGUUAA |
| B2M | B2M-14 AsCpf1 | AGCAGCUUACAAAAGAAUGU |
| B2M | B2M-1 AsCpf1 RR | UGAAGCUGACAGCAUUCGGG |
| B2M | B2M-2 AsCpf1 RR | GGCCGAGAUGUCUCGCUCCG |
| B2M | B2M-3 AsCpf1 RR | UGGCCUUAGCUGUGCUCGCG |
| B2M | B2M-4 AsCpf1 RR | GCGUGAGUCUCUCCUACCCU |
| B2M | B2M-5 AsCpf1 RR | GGCCAGAAAGAGAGAGUAGC |
| B2M | B2M-6 AsCpf1 RR | CGAUAUUCCUCAGGUACUCC |
| B2M | B2M-7 AsCpf1 RR | UCAGGUACUCCAAAGAUUCA |
| B2M | B2M-8 AsCpf1 RR | AAGAUUCAGGUUUACUCACG |
| B2M | B2M-9 AsCpf1 RR | GGUUUACUCACGUCAUCCAG |
| B2M | B2M-10 AsCpf1 RR | GCAGAGAAUGGAAAGUCAAA |
| B2M | B2M-11 AsCpf1 RR | UUCUCUGCUGGAUGACGUGA |
| B2M | B2M-12 AsCpf1 RR | AUUCUCUGCUGGAUGACGUG |
| B2M | B2M-13 AsCpf1 RR | UGAAUUGCUAUGUGUCUGGG |
| B2M | B2M-14 AsCpf1 RR | GGAAAUUUGACUUUCCAUUC |
| B2M | B2M-15 AsCpf1 RR | UCCAUCCGACAUUGAAGUUG |
| B2M | B2M-16 AsCpf1 RR | UCCGACAUUGAAGUUGACUU |
| B2M | B2M-17 AsCpf1 RR | ACAUUGAAGUUGACUUACUG |
| B2M | B2M-18 AsCpf1 RR | AUGUCGGAUGGAUGAAACCC |
| B2M | B2M-19 AsCpf1 RR | GUAAGUCAACUUCAAUGUCG |
| B2M | B2M-20 AsCpf1 RR | UUCUUCAGUAAGUCAACUUC |
| B2M | B2M-21 AsCpf1 RR | AUUCUCUCUCCAUUCUUCAG |
| B2M | B2M-22 AsCpf1 RR | CUUUUUCAAUUCUCUCUCCA |
| B2M | B2M-23 AsCpf1 RR | GACUUGUCUUUCAGCAAGGA |
| B2M | B2M-24 AsCpf1 RR | GCAAGGACUGGUCUUUCUAU |
| B2M | B2M-25 AsCpf1 RR | GUGUAGUACAAGAGAUAGAA |
| B2M | B2M-26 AsCpf1 RR | CCCCCACUGAAAAAGAUGAG |
| B2M | B2M-27 AsCpf1 RR | CACUGAAAAAGAUGAGUAUG |
| B2M | B2M-28 AsCpf1 RR | ACUGAAAAAGAUGAGUAUGC |
| B2M | B2M-29 AsCpf1 RR | GUGGGGGUGAAUUCAGUGUA |
| B2M | B2M-30 AsCpf1 RR | CACGGCAGGCAUACUCAUCU |
| B2M | B2M-31 AsCpf1 RR | ACUUAACUAUCUUGGGCUGU |
| B2M | B2M-32 AsCpf1 RR | GCAGCUUACAAAAGAAUGUA |
| B2M | B2M-1 AsCpf1 RVR | CAGCGUGAGUCUCUCCUACC |
| B2M | B2M-2 AsCpf1 RVR | UGUCUGGGUUUCAUCCAUCC |
| B2M | B2M-3 AsCpf1 RVR | UCUUGUACUACACUGAAUUC |
| B2M | B2M-4 AsCpf1 RVR | CCUGCCGUGUGAACCAUGUG |
| B2M | B2M-5 AsCpf1 RVR | UUGGGCUGUGACAAAGUCAC |

TABLE 7

| | Within the first 500 bp of coding | | |
|---|---|---|---|
| | sequence | | |
| AsCpf1 | | | Target |
| gRNA | DNA | | Site |
| Name | Strand | Targeting Domain | Length |
| B2M-c1 | + | AAUUCUCUCUCCAUUCUU | 18 |
| B2M-c2 | + | AAUUCUCUCUCCAUUCUUC | 19 |
| B2M-c3 | + | AAUUCUCUCUCCAUUCUUCA | 20 |
| B2M-c4 | + | AAUUCUCUCUCCAUUCUUCAG | 21 |
| B2M-c5 | + | AAUUCUCUCUCCAUUCUUCAGU | 22 |
| B2M-c6 | + | AAUUCUCUCUCCAUUCUUCAGUA | 23 |
| B2M-c7 | + | AAUUCUCUCUCCAUUCUUCAGUAA | 24 |
| B2M-c8 | + | ACUUUCCAUUCUCUGCUG | 18 |
| B2M-c9 | + | ACUUUCCAUUCUCUGCUGG | 19 |
| B2M-c10 | + | ACUUUCCAUUCUCUGCUGGA | 20 |
| B2M-c11 | + | ACUUUCCAUUCUCUGCUGGAU | 21 |
| B2M-c12 | + | ACUUUCCAUUCUCUGCUGGAUG | 22 |
| B2M-c13 | + | ACUUUCCAUUCUCUGCUGGAUGA | 23 |
| B2M-c14 | + | ACUUUCCAUUCUCUGCUGGAUGAC | 24 |
| B2M-c15 | − | AGCAAGGACUGGUCUUUC | 18 |
| B2M-c16 | − | AGCAAGGACUGGUCUUUCU | 19 |
| B2M-c17 | − | AGCAAGGACUGGUCUUUCUA | 20 |
| B2M-c18 | − | AGCAAGGACUGGUCUUUCUAU | 21 |
| B2M-c19 | − | AGCAAGGACUGGUCUUUCUAUC | 22 |
| B2M-c20 | − | AGCAAGGACUGGUCUUUCUAUCU | 23 |
| B2M-c21 | − | AGCAAGGACUGGUCUUUCUAUCUC | 24 |
| B2M-c22 | + | AGUGGGGGUGAAUUCAGU | 18 |
| B2M-c23 | + | AGUGGGGGUGAAUUCAGUG | 19 |
| B2M-c24 | + | AGUGGGGGUGAAUUCAGUGU | 20 |
| B2M-c25 | + | AGUGGGGGUGAAUUCAGUGUA | 21 |
| B2M-c26 | + | AGUGGGGGUGAAUUCAGUGUAG | 22 |
| B2M-c27 | + | AGUGGGGGUGAAUUCAGUGUAGU | 23 |
| B2M-c28 | + | AGUGGGGGUGAAUUCAGUGUAGUA | 24 |
| B2M-c29 | − | AUCCAUCCGACAUUGAAG | 18 |
| B2M-c30 | − | AUCCAUCCGACAUUGAAGU | 19 |
| B2M-c31 | − | AUCCAUCCGACAUUGAAGUU | 20 |
| B2M-c32 | − | AUCCAUCCGACAUUGAAGUUG | 21 |
| B2M-c33 | − | AUCCAUCCGACAUUGAAGUUGA | 22 |
| B2M-c34 | − | AUCCAUCCGACAUUGAAGUUGAC | 23 |
| B2M-c35 | − | AUCCAUCCGACAUUGAAGUUGACU | 24 |
| B2M-c36 | + | CAAUUCUCUCUCCAUUCU | 18 |
| B2M-c37 | + | CAAUUCUCUCUCCAUUCUU | 19 |
| B2M-c38 | + | CAAUUCUCUCUCCAUUCUUC | 20 |
| B2M-c39 | + | CAAUUCUCUCUCCAUUCUUCA | 21 |
| B2M-c40 | + | CAAUUCUCUCUCCAUUCUUCAG | 22 |
| B2M-c41 | + | CAAUUCUCUCUCCAUUCUUCAGU | 23 |
| B2M-c42 | + | CAAUUCUCUCUCCAUUCUUCAGUA | 24 |
| B2M-c43 | + | CAGUGGGGGUGAAUUCAG | 18 |
| B2M-c44 | + | CAGUGGGGGUGAAUUCAGU | 19 |
| B2M-c45 | + | CAGUGGGGGUGAAUUCAGUG | 20 |
| B2M-c46 | + | CAGUGGGGGUGAAUUCAGUGU | 21 |
| B2M-c47 | + | CAGUGGGGGUGAAUUCAGUGUA | 22 |
| B2M-c48 | + | CAGUGGGGGUGAAUUCAGUGUAG | 23 |
| B2M-c49 | + | CAGUGGGGGUGAAUUCAGUGUAGU | 24 |
| B2M-c50 | + | CAUUCUCUGCUGGAUGAC | 18 |
| B2M-c51 | + | CAUUCUCUGCUGGAUGACG | 19 |
| B2M-c52 | + | CAUUCUCUGCUGGAUGACGU | 20 |
| B2M-c53 | + | CAUUCUCUGCUGGAUGACGUG | 21 |
| B2M-c54 | + | CAUUCUCUGCUGGAUGACGUGA | 22 |
| B2M-c55 | + | CAUUCUCUGCUGGAUGACGUGAG | 23 |
| B2M-c56 | + | CAUUCUCUGCUGGAUGACGUGAGU | 24 |
| B2M-c57 | − | CCCGAUAUUCCUCAGGUA | 18 |
| B2M-c58 | − | CCCGAUAUUCCUCAGGUAC | 19 |
| B2M-c59 | − | CCCGAUAUUCCUCAGGUACU | 20 |
| B2M-c60 | − | CCCGAUAUUCCUCAGGUACUC | 21 |
| B2M-c61 | − | CCCGAUAUUCCUCAGGUACUCC | 22 |
| B2M-c62 | − | CCCGAUAUUCCUCAGGUACUCCA | 23 |
| B2M-c63 | − | CCCGAUAUUCCUCAGGUACUCCAA | 24 |
| B2M-c64 | − | CCGAUAUUCCUCAGGUAC | 18 |
| B2M-c65 | − | CCGAUAUUCCUCAGGUACU | 19 |
| B2M-c66 | − | CCGAUAUUCCUCAGGUACUC | 20 |
| B2M-c67 | − | CCGAUAUUCCUCAGGUACUCC | 21 |
| B2M-c68 | − | CCGAUAUUCCUCAGGUACUCCA | 22 |
| B2M-c69 | − | CCGAUAUUCCUCAGGUACUCCAA | 23 |
| B2M-c70 | − | CCGAUAUUCCUCAGGUACUCCAAA | 24 |
| B2M-c71 | − | CUCACGUCAUCCAGCAGA | 18 |
| B2M-c72 | − | CUCACGUCAUCCAGCAGAG | 19 |
| B2M-c73 | − | CUCACGUCAUCCAGCAGAGA | 20 |
| B2M-c74 | − | CUCACGUCAUCCAGCAGAGAA | 21 |
| B2M-c75 | − | CUCACGUCAUCCAGCAGAGAAU | 22 |
| B2M-c76 | − | CUCACGUCAUCCAGCAGAGAAUG | 23 |
| B2M-c77 | − | CUCACGUCAUCCAGCAGAGAAUGG | 24 |
| B2M-c78 | − | CUGAAUUGCUAUGUGUCU | 18 |
| B2M-c79 | − | CUGAAUUGCUAUGUGUCUG | 19 |
| B2M-c80 | − | CUGAAUUGCUAUGUGUCUGG | 20 |
| B2M-c81 | − | CUGAAUUGCUAUGUGUCUGGG | 21 |
| B2M-c82 | − | CUGAAUUGCUAUGUGUCUGGGU | 22 |
| B2M-c83 | − | CUGAAUUGCUAUGUGUCUGGGUU | 23 |
| B2M-c84 | − | CUGAAUUGCUAUGUGUCUGGGUUU | 24 |
| B2M-c85 | + | GAGUACCUGAGGAAUAUC | 18 |
| B2M-c86 | + | GAGUACCUGAGGAAUAUCG | 19 |
| B2M-c87 | + | GAGUACCUGAGGAAUAUCGG | 20 |
| B2M-c88 | + | GAGUACCUGAGGAAUAUCGGG | 21 |
| B2M-c89 | + | GAGUACCUGAGGAAUAUCGGGA | 22 |
| B2M-c90 | + | GAGUACCUGAGGAAUAUCGGGAA | 23 |
| B2M-c91 | + | GAGUACCUGAGGAAUAUCGGGAAA | 24 |
| B2M-c92 | − | UAUCUCUUGUACUACACU | 18 |
| B2M-c93 | − | UAUCUCUUGUACUACACUG | 19 |
| B2M-c94 | − | UAUCUCUUGUACUACACUGA | 20 |
| B2M-c95 | − | UAUCUCUUGUACUACACUGAA | 21 |
| B2M-c96 | − | UAUCUCUUGUACUACACUGAAU | 22 |
| B2M-c97 | − | UAUCUCUUGUACUACACUGAAUU | 23 |
| B2M-c98 | − | UAUCUCUUGUACUACACUGAAUUC | 24 |
| B2M-c99 | + | UCAAUUCUCUCUCCAUUC | 18 |
| B2M-c100 | + | UCAAUUCUCUCUCCAUUCU | 19 |
| B2M-c101 | + | UCAAUUCUCUCUCCAUUCUU | 20 |
| B2M-c102 | + | UCAAUUCUCUCUCCAUUCUUC | 21 |
| B2M-c103 | + | UCAAUUCUCUCUCCAUUCUUCA | 22 |
| B2M-c104 | + | UCAAUUCUCUCUCCAUUCUUCAG | 23 |
| B2M-c105 | + | UCAAUUCUCUCUCCAUUCUUCAGU | 24 |
| B2M-c106 | − | UCACAGCCCAAGAUAGUU | 18 |
| B2M-c107 | − | UCACAGCCCAAGAUAGUUA | 19 |
| B2M-c108 | − | UCACAGCCCAAGAUAGUUAA | 20 |
| B2M-c109 | − | UCACAGCCCAAGAUAGUUAAG | 21 |
| B2M-c110 | − | UCACAGCCCAAGAUAGUUAAGU | 22 |
| B2M-c111 | − | UCACAGCCCAAGAUAGUUAAGUG | 23 |
| B2M-c112 | − | UCACAGCCCAAGAUAGUUAAGUGG | 24 |
| B2M-c113 | + | UCAGUGGGGGUGAAUUCA | 18 |
| B2M-c114 | + | UCAGUGGGGGUGAAUUCAG | 19 |
| B2M-c115 | + | UCAGUGGGGGUGAAUUCAGU | 20 |
| B2M-c116 | + | UCAGUGGGGGUGAAUUCAGUG | 21 |
| B2M-c117 | + | UCAGUGGGGGUGAAUUCAGUGU | 22 |
| B2M-c118 | + | UCAGUGGGGGUGAAUUCAGUGUA | 23 |
| B2M-c119 | + | UCAGUGGGGGUGAAUUCAGUGUAG | 24 |
| B2M-c120 | − | UGGCCUGGAGGCUAUCCA | 18 |
| B2M-c121 | − | UGGCCUGGAGGCUAUCCAG | 19 |
| B2M-c122 | − | UGGCCUGGAGGCUAUCCAGC | 20 |
| B2M-c123 | − | UGGCCUGGAGGCUAUCCAGCG | 21 |
| B2M-c124 | − | UGGCCUGGAGGCUAUCCAGCGU | 22 |
| B2M-c125 | − | UGGCCUGGAGGCUAUCCAGCGUG | 23 |
| B2M-c126 | − | UGGCCUGGAGGCUAUCCAGCGUGA | 24 |

TABLE 8

| | The rest of the coding sequence | | |
|---|---|---|---|
| AsCpf1 | | | Target |
| gRNA | DNA | | Site |
| Name | Strand | Targeting Domain | Length |
| B2M-c127 | − | AUAGAUCGAGACAUGUAA | 18 |
| B2M-c128 | − | AUAGAUCGAGACAUGUAAG | 19 |
| B2M-c129 | − | AUAGAUCGAGACAUGUAAGC | 20 |
| B2M-c130 | − | AUAGAUCGAGACAUGUAAGCA | 21 |
| B2M-c131 | − | AUAGAUCGAGACAUGUAAGCAG | 22 |
| B2M-c132 | − | AUAGAUCGAGACAUGUAAGCAGC | 23 |
| B2M-c133 | − | AUAGAUCGAGACAUGUAAGCAGCA | 24 |
| B2M-c134 | − | CAUAGAUCGAGACAUGUA | 18 |
| B2M-c135 | − | CAUAGAUCGAGACAUGUAA | 19 |
| B2M-c136 | − | CAUAGAUCGAGACAUGUAAG | 20 |
| B2M-c137 | − | CAUAGAUCGAGACAUGUAAGC | 21 |
| B2M-c138 | − | CAUAGAUCGAGACAUGUAAGCA | 22 |
| B2M-c139 | − | CAUAGAUCGAGACAUGUAAGCAG | 23 |
| B2M-c140 | − | CAUAGAUCGAGACAUGUAAGCAGC | 24 |
| B2M-c141 | − | CUCCACUGUCUUUUUCAU | 18 |
| B2M-c142 | − | CUCCACUGUCUUUUUCAUA | 19 |
| B2M-c143 | − | CUCCACUGUCUUUUUCAUAG | 20 |
| B2M-c144 | − | CUCCACUGUCUUUUUCAUAGA | 21 |
| B2M-c145 | − | CUCCACUGUCUUUUUCAUAGAU | 22 |
| B2M-c146 | − | CUCCACUGUCUUUUUCAUAGAUC | 23 |
| B2M-c147 | − | CUCCACUGUCUUUUUCAUAGAUCG | 24 |
| B2M-c148 | − | UCAUAGAUCGAGACAUGU | 18 |
| B2M-c149 | − | UCAUAGAUCGAGACAUGUA | 19 |
| B2M-c150 | − | UCAUAGAUCGAGACAUGUAA | 20 |
| B2M-c151 | − | UCAUAGAUCGAGACAUGUAAG | 21 |
| B2M-c152 | − | UCAUAGAUCGAGACAUGUAAGC | 22 |
| B2M-c153 | − | UCAUAGAUCGAGACAUGUAAGCA | 23 |
| B2M-c154 | − | UCAUAGAUCGAGACAUGUAAGCAG | 24 |
| B2M-c155 | − | UCCACUGUCUUUUUCAUA | 18 |
| B2M-c156 | − | UCCACUGUCUUUUUCAUAG | 19 |
| B2M-c157 | − | UCCACUGUCUUUUUCAUAGA | 20 |
| B2M-c158 | − | UCCACUGUCUUUUUCAUAGAU | 21 |
| B2M-c159 | − | UCCACUGUCUUUUUCAUAGAUC | 22 |
| B2M-c160 | − | UCCACUGUCUUUUUCAUAGAUCG | 23 |
| B2M-c161 | − | UCCACUGUCUUUUUCAUAGAUCGA | 24 |
| B2M-c162 | − | UCUCCACUGUCUUUUUCA | 18 |
| B2M-c163 | − | UCUCCACUGUCUUUUUCAU | 19 |
| B2M-c164 | − | UCUCCACUGUCUUUUUCAUA | 20 |
| B2M-c165 | − | UCUCCACUGUCUUUUUCAUAG | 21 |
| B2M-c166 | − | UCUCCACUGUCUUUUUCAUAGA | 22 |
| B2M-c167 | − | UCUCCACUGUCUUUUUCAUAGAU | 23 |
| B2M-c168 | − | UCUCCACUGUCUUUUUCAUAGAUC | 24 |
| B2M-c169 | − | UUCUCCACUGUCUUUUUC | 18 |
| B2M-c170 | − | UUCUCCACUGUCUUUUUCA | 19 |
| B2M-c171 | − | UUCUCCACUGUCUUUUUCAU | 20 |
| B2M-c172 | − | UUCUCCACUGUCUUUUUCAUA | 21 |
| B2M-c173 | − | UUCUCCACUGUCUUUUUCAUAG | 22 |
| B2M-c174 | − | UUCUCCACUGUCUUUUUCAUAGA | 23 |
| B2M-c175 | − | UUCUCCACUGUCUUUUUCAUAGAU | 24 |
| B2M-c176 | − | UUUCUCCACUGUCUUUUU | 18 |
| B2M-c177 | − | UUUCUCCACUGUCUUUUUC | 19 |
| B2M-c178 | − | UUUCUCCACUGUCUUUUUCA | 20 |
| B2M-c179 | − | UUUCUCCACUGUCUUUUUCAU | 21 |
| B2M-c180 | − | UUUCUCCACUGUCUUUUUCAUA | 22 |
| B2M-c181 | − | UUUCUCCACUGUCUUUUUCAUAG | 23 |
| B2M-c182 | − | UUUCUCCACUGUCUUUUUCAUAGA | 24 |
| B2M-c183 | − | UUUUCUCCACUGUCUUUU | 18 |
| B2M-c184 | − | UUUUCUCCACUGUCUUUUU | 19 |
| B2M-c185 | − | UUUUCUCCACUGUCUUUUUC | 20 |
| B2M-c186 | − | UUUUCUCCACUGUCUUUUUCA | 21 |
| B2M-c187 | − | UUUUCUCCACUGUCUUUUUCAU | 22 |
| B2M-c188 | − | UUUUCUCCACUGUCUUUUUCAUA | 23 |
| B2M-c189 | − | UUUUCUCCACUGUCUUUUUCAUAG | 24 |

TABLE 9

| Gene | gRNA Name | Targeting Domain |
|---|---|---|
| CITTA | CITTA AsCpf1-1 | UCGAGUUGGAUGUGGAAGGU |
| CITTA | CITTA AsCpf1-2 | UUUUCAUCCCCACUUCACAC |
| CITTA | CITTA AsCpf1-3 | CCUCGGGGGAGAGAGAGGUG |
| CITTA | CITTA AsCpf1-4 | UGGGCUCAGGUGCUUCCUCA |
| CITTA | CITTA AsCpf1-5 | UCAAAGUAGAGCACAUAGGA |
| CITTA | CITTA AsCpf1-6 | CCAUCAAAAGUCCUUUUUGG |
| CITTA | CITTA AsCpf1-7 | GUGUCUACACUUAGCCUUUC |
| CITTA | CITTA AsCpf1-8 | GGGUGAAAUUUCCCAACUUU |
| CITTA | CITTA AsCpf1-9 | CCGGCCUUUUUACCUUGGGG |
| CITTA | CITTA AsCpf1-10 | UCUGCAGCCUUCCCAGAGGA |
| CITTA | CITTA AsCpf1-11 | AAAGAGGACCUUCUAAAAAU |
| CITTA | CITTA AsCpf1-12 | GGGUUUAUUUUCAUCCCCAC |
| CITTA | CITTA AsCpf1-13 | AUCCCCACUUCACACUGCAU |
| CITTA | CITTA AsCpf1-14 | CUUGUCUGGGCAGCGGAACU |
| CITTA | CITTA AsCpf1-15 | AGAAAAACCAGAGACCAACU |
| CITTA | CITTA AsCpf1-16 | AGUCUGAGUUAGAACAUUGU |
| CITTA | CITTA AsCpf1-17 | UGACUUUUCUGCCCAACUUC |
| CITTA | CITTA AsCpf1-18 | AGGAGCCAUGUGGGGGCAGG |
| CITTA | CITTA AsCpf1-19 | AAACUGUGCUUCCCCCUGGG |
| CITTA | CITTA AsCpf1-20 | GAAGGUCCUCUUUGAAACUG |
| CITTA | CITTA AsCpf1-21 | AGACACCUGUUUAGUGUCUA |
| CITTA | CITTA AsCpf1-22 | AAAGGCUAAGUGUAGACACU |
| CITTA | CITTA AsCpf1-23 | CCAAAAAGACACAGACCGCG |
| CITTA | CITTA AsCpf1-24 | CCAACUUUCAGGUUAUCCCU |
| CITTA | CITTA AsCpf1-25 | AGUAAGUUUGUGGUGGGUGG |
| CITTA | CITTA AsCpf1-26 | AAAUCCUGCAUGCAGUGUGA |
| CITTA | CITTA AsCpf1-27 | AAAAAUGAACUUACCCAGAU |
| CITTA | CITTA AsCpf1-28 | ACCCCAAAGCUCACCAUCUG |
| CITTA | CITTA AsCpf1-29 | UGCCCAACUUCUGCUGGCAU |
| CITTA | CITTA AsCpf1-30 | AUGGCAAAAAGAUCAGGAAU |
| CITTA | CITTA AsCpf1-31 | GUAAAUGGGCAGUAUUUUUA |
| CITTA | CITTA AsCpf1-32 | CUCCCAGAACCCGACACAGA |
| CITTA | CITTA AsCpf1-33 | AGGUUAUCCCUACCUACCAA |
| CITTA | CITTA AsCpf1-34 | CCUUGGGGCUCUGACAGGUA |
| CITTA | CITTA AsCpf1-35 | UGGUGGGUGGGGAGGUCUUG |
| CITTA | CITTA AsCpf1 RR-1 | UUUUUUAAACCACUUGGAGC |
| CITTA | CITTA AsCpf1 RR-2 | UCCCCACUUCACACUGCAUG |
| CITTA | CITTA AsCpf1 RR-3 | AGGGACUUUUCCUCCCAGAA |
| CITTA | CITTA AsCpf1 RR-4 | GCAGACCUGAAGCACUGGAA |
| CITTA | CITTA AsCpf1 RR-5 | GUAAGUUUGUGGUGGGUGGG |
| CITTA | CITTA AsCpf1 RR-6 | AGGCAGCUCACAGUGUGCCA |
| CITTA | CITTA AsCpf1 RR-7 | AGGACUCCCAGCUGGAGGGC |
| CITTA | CITTA AsCpf1 RR-8 | CGCCCUGCUGGGUCCUACCU |
| CITTA | CITTA AsCpf1 RR-9 | CAAGGAUGCCUUCGGAUGCC |
| CITTA | CITTA AsCpf1 RR-10 | ACUCGAGAAAAUAUUCCUGA |
| CITTA | CITTA AsCpf1 RR-11 | AUCUCAGCUGGUGGGAGAUG |
| CITTA | CITTA AsCpf1 RR-12 | CCCUGGGCCACCACCUUCCA |
| CITTA | CITTA AsCpf1 RR-13 | CUGGGCCACCACCUUCCACA |
| CITTA | CITTA AsCpf1 RR-14 | AACUGGUGACUGGUUAGUGA |
| CITTA | CITTA AsCpf1 RR-15 | UCGGGGGAGAGAGAGGUGAA |
| CITTA | CITTA AsCpf1 RR-16 | UGAUCUUUUUGCCAUCAAAA |
| CITTA | CITTA AsCpf1 RR-17 | CCUGGGCCACCACCUUCCAC |
| CITTA | CITTA AsCpf1 RR-18 | UCCCAGAACCCGACACAGAC |
| CITTA | CITTA AsCpf1 RR-19 | AGUGCUUCAGGUCUGCCGGA |
| CITTA | CITTA AsCpf1 RR-20 | UUCAUCUCCCACCAGCUGAG |
| CITTA | CITTA AsCpf1 RR-21 | AAGAGGACCUUCUAAAAAUA |
| CITTA | CITTA AsCpf1 RR-22 | CUGUGCCUCUACCACUUCUA |
| CITTA | CITTA AsCpf1 RR-23 | CAGAGGAGCUUCCGGCAGAC |
| CITTA | CITTA AsCpf1 RR-24 | AACUUUCAGGUUAUCCCUAC |
| CITTA | CITTA AsCpf1 RR-25 | AAAGCUCACCAUCUGAGCUC |
| CITTA | CITTA AsCpf1 RR-26 | CAGAAGAGAUGCAUGCACUG |
| CITTA | CITTA AsCpf1 RR-27 | CUCCCAGGCAGCUCACAGUG |
| CITTA | CITTA AsCpf1 RR-28 | CACCCACCACAAACUUACUG |
| CITTA | CITTA AsCpf1 RR-29 | GGAGUCUGGCAGCCCCUCCU |
| CITTA | CITTA AsCpf1 RR-30 | GGCAGACCUGAAGCACUGGA |
| CITTA | CITTA AsCpf1 RR-31 | GGACUCCCAGCUGGAGGGCC |
| CITTA | CITTA AsCpf1 RR-32 | AACUCCAUGGUGGCACACUG |
| CITTA | CITTA AsCpf1 RR-33 | UCACCUUCCAUGUCACACAA |
| CITTA | CITTA AsCpf1 RR-34 | AAGGCAUCCUUGGGGAAGCU |
| CITTA | CITTA AsCpf1 RR-35 | ACAUCCAACUCGAGAAAAUA |
| CITTA | CITTA AsCpf1 RR-36 | CUCGGGGGAGAGAGAGGUGA |
| CITTA | CITTA AsCpf1 RR-37 | AGAGGAGCUUCCGGCAGACC |
| CITTA | CITTA AsCpf1 RR-38 | GACACCUGUUUAGUGUCUAC |
| CITTA | CITTA AsCpf1 RR-39 | AGAAGAGAUGCAUGCACUGA |
| CITTA | CITTA AsCpf1 RR-40 | GUUCCGCUGCCCAGACAAGG |
| CITTA | CITTA AsCpf1 RR-41 | UCCCAGGCAGCUCACAGUGU |
| CITTA | CITTA AsCpf1 RR-42 | CACUUCACACUGCAUGCAGG |
| CITTA | CITTA AsCpf1 RR-43 | CCUCUCUCUCCCCCGAGGGA |
| CITTA | CITTA AsCpf1 RR-44 | UGGUCUCUUCAUCACCUUCC |
| CITTA | CITTA AsCpf1 RR-45 | GCAGGCUGUUGUGUGACAUG |
| CITTA | CITTA AsCpf1 RR-46 | ACCAGCUGAGAUGGAACGUU |
| CITTA | CITTA AsCpf1 RR-47 | UGGUGGCACACUGUGAGCUG |
| CITTA | CITTA AsCpf1 RR-48 | GCUGGGAGUCCUGGAAGACA |
| CITTA | CITTA AsCpf1 RR-49 | GGAGCUGCUGCCUGGCUGGG |
| CITTA | CITTA AsCpf1 RR-50 | UCUCAGCUGGUGGGAGAUGA |
| CITTA | CITTA AsCpf1 RR-51 | GUGCUUCAGGUCUGCCGGAA |
| CITTA | CITTA AsCpf1 RR-52 | CAACUUUCAGGUUAUCCCUA |
| CITTA | CITTA AsCpf1 RR-53 | GGUAGCCACCUUCUAGGGGC |
| CITTA | CITTA AsCpf1 RR-54 | UGUCACACAACAGCCUGCUG |
| CITTA | CITTA AsCpf1 RR-55 | GCUGCCCAGACAAGGAAAAG |
| CITTA | CITTA AsCpf1 RR-56 | CCAAGGAUGCCUUCGGAUGC |
| CITTA | CITTA AsCpf1 RR-57 | GAUAGUUUAAGUCUGAGUUA |
| CITTA | CITTA AsCpf1 RR-58 | AGAACCCGACACAGACACCA |
| CITTA | CITTA AsCpf1 RR-59 | AAAAAGGACUUUUGAUGGCA |
| CITTA | CITTA AsCpf1 RR-60 | GGUUAUCCCUACCUACCAAC |
| CITTA | CITTA AsCpf1 RR-61 | GAGGGAAAUCAGGUGUCGCC |
| CITTA | CITTA AsCpf1 RR-62 | UACUCUCACCGAUCACUUCA |
| CITTA | CITTA AsCpf1 RR-63 | CGAGGGAAAUCAGGUGUCGC |
| CITTA | CITTA AsCpf1 RR-64 | UCACCGAUAUUGGCAUAAGC |
| CITTA | CITTA AsCpf1 RR-65 | ACUUCACACUGCAUGCAGGA |
| CITTA | CITTA AsCpf1 RR-66 | AGUGGUUUAAAAAAUGAACU |
| CITTA | CITTA AsCpf1 RR-67 | AAGGUAAAAAGGCCGGGAAA |
| CITTA | CITTA AsCpf1 RR-68 | GGUCUGCCGGAAGCUCCUCU |
| CITTA | CITTA AsCpf1 RR-69 | CCGAGGGAAAUCAGGUGUCG |
| CITTA | CITTA AsCpf1 RR-70 | UCCUCGUGCCCUCAGCUUCC |
| CITTA | CITTA AsCpf1 RR-71 | CAUCCAACUCGAGAAAAUAU |
| CITTA | CITTA AsCpf1 RR-72 | AGCCCACCUGCCCUGCACAC |
| CITTA | CITTA AsCpf1 RR-73 | CGGCCUUUUUACCUUGGGGC |
| CITTA | CITTA AsCpf1 RR-74 | AACCCCAGCCCACCUGCCCU |
| CITTA | CITTA AsCpf1 RR-75 | AAUCCUGCAUGCAGUGUGAA |
| CITTA | CITTA AsCpf1 RR-76 | UACACAAUGCGUUGCCUGGC |
| CITTA | CITTA AsCpf1 RR-77 | CCCCAAAGCUCACCAUCUGA |
| CITTA | CITTA AsCpf1 RR-78 | UGGGCCACCACCUUCCACAU |
| CITTA | CITTA AsCpf1 RR-79 | GCCAGGUCCAUCUGGUCAUA |
| CITTA | CITTA AsCpf1 RR-80 | AUGUCACACAACAGCCUGCU |
| CITTA | CITTA AsCpf1 RR-81 | GGCCUUUUUACCUUGGGGCU |
| CITTA | CITTA AsCpf1 RR-82 | UACCUACCAACGCACUACAA |
| CITTA | CITTA AsCpf1 RR-83 | UCUGGUCAUAGAAGUGGUAG |
| CITTA | CITTA AsCpf1 RR-84 | CACUGCAUGCAGGAUUUGAA |
| CITTA | CITTA AsCpf1 RR-85 | AGCUGGAGGGCCUGAGCAAG |
| CITTA | CITTA AsCpf1 RR-86 | AAGGAUGCCUUCGGAUGCCC |
| CITTA | CITTA AsCpf1 RR-87 | UGUGCCUCUACCACUUCUAU |
| CITTA | CITTA AsCpf1 RR-88 | UAGAAGGUGGCUACCUGGAG |
| CITTA | CITTA AsCpf1 RR-89 | UUGUCUGGGCAGCGGAACUG |
| CITTA | CITTA AsCpf1 RR-90 | ACCCACCACAAACUUACUGA |
| CITTA | CITTA AsCpf1 RR-91 | UCCAAGGGACUUUUCCUCCC |
| CITTA | CITTA AsCpf1 RR-92 | UCUGGUCCUAUGUGCUCUAC |
| CITTA | CITTA AsCpf1 RR-93 | CUGCCCAGACAAGGAAAAGC |
| CITTA | CITTA AsCpf1 RR-94 | AGCCAGGCAGCAGCUCCCGG |
| CITTA | CITTA AsCpf1 RR-95 | GAUGCCCAGCUCAGAAGCAC |
| CITTA | CITTA AsCpf1 RR-96 | UCUCCCACCAGCUGAGAUGG |
| CITTA | CITTA AsCpf1 RVR-1 | UGAAGAUCAGUGCAUGCAUC |
| CITTA | CITTA AsCpf1 RVR-2 | CCUACCUACCAACGCACUAC |
| CITTA | CITTA AsCpf1 RVR-3 | ACCAGAUGGACCUGGCUGGA |
| CITTA | CITTA AsCpf1 RVR-4 | UGCUCUACUUUGAGAAAAAC |
| CITTA | CITTA AsCpf1 RVR-5 | UCUUCCAGGACUCCCAGCUG |
| CITTA | CITTA AsCpf1 RVR-6 | CUGUUCCCAGAAGAGAUGCA |
| CITTA | CITTA AsCpf1 RVR-7 | GGUGAGGAAGCACCUGAGCC |
| CITTA | CITTA AsCpf1 RVR-8 | CCAAUAUCGGUGAGGAAGCA |
| CITTA | CITTA AsCpf1 RVR-9 | GAGAUGCCAGCAGAAGUUGG |

Knock-out and/or knock down of FAS, BID, CTLA4, PDCD1, CBLB, PTPN6, B2M, TRAC, CIITA and TRBC may be useful in a variety of settings, including without limitation in the context of adoptive immunotherapy for treating cancer and non-cancer diseases, e.g., an autoimmune disorder. According to certain embodiments of this disclosure, FAS, BID, CTLA4, PDCD1, CBLB, PTPN6, B2M, TRAC, CIITA and TRBC are knocked out in an immune cell, such as a T cell, that will be used in therapy. As one non-limiting example, the T cell may express an engineered receptor such as a chimeric antigen receptor (CAR) or a heterologous T cell receptor (TCR), which receptor may be configured to recognize an antigen on a cell or tissue that is implicated in a pathology such as a tumor cell. Whether or not they express an engineered receptor, TCR, MHCI and/or MHCII knockout T cells according the present disclosure may be employed in the targeting of a tissue or organ in which GvH or HvG response may present a safety or efficacy concern.

TCR, MHCI and/or MHCII knock-out and/or knock down cells may be employed in “allogeneic” cell therapies, in which cells are harvested from a subject, modified to knock-out or knock-down, e.g., disrupt, FAS, BID, CTLA4, PDCD1, CBLB, PTPN6, B2M, TRAC, CIITA and TRBC expression, and then returned to a different subject. In either approach, between harvesting and administration TCR, MHCI and/or MHCII knock-out and/or knock down cells of this disclosure may be manipulated in a variety of ways, such as expanded, stimulated, purified or sorted, transduced with a transgene, frozen and/or thawed.

Knocking out or knocking down the FAS, BID, CTLA4, PDCD1, CBLB, PTPN6, B2M, TRAC, CIITA and/or TRBC genes as described herein can: (1) prevent GvH response; (2) prevent HvG response; and/or (3) improve T cell safety and efficacy. Knocking down the expression of the FAS, BID, CTLA4, PDCD1, CBLB, PTPN6, B2M, TRAC, CIITA and/or TRBC proteins as described herein can similarly: (1) prevent GvH response; (2) prevent HvG response; and/or (3) improve T cell safety and efficacy.

In certain embodiments, a presently disclosed method comprises independently knocking out and/or knocking down one or more genes selected from the group consisting of B2M, TRAC, CIITA and TRBC in a T cell. In certain embodiments, a presently disclosed method comprises independently knocking out and/or knocking down two genes selected from the group consisting of B2M, TRAC, CIITA and TRBC in a T cell. In certain embodiments, a presently disclosed method comprises independently knocking out and/or knocking down three genes selected from the group consisting of B2M, TRAC, CIITA and TRBC in a T cell. In certain embodiments, a presently disclosed method comprises independently knocking out and/or knocking down all four genes B2M, TRAC, CIITA and TRBC in a T cell.

In certain embodiments, a presently disclosed method comprises knocking out and/or knocking down the B2M gene in a T cell. In certain embodiments, a presently disclosed method comprises knocking out and/or knocking down the TRAC gene in a T cell. In certain embodiments, a presently disclosed method comprises knocking out and/or knocking down the CIITA gene in a T cell. In certain embodiments, a presently disclosed method comprises knocking out and/or knocking down the TRBC gene in a T cell. In certain embodiments, a presently disclosed method comprises knocking out and/or knocking down the B2M and TRAC genes in a T cell. In certain embodiments, a presently disclosed method comprises knocking out and/or knocking down the B2M and CIITA genes in a T cell. In certain embodiments, a presently disclosed method comprises knocking out and/or knocking down the B2M and TRBC genes in a T cell. In certain embodiments, a presently disclosed method comprises knocking out and/or knocking down the TRAC and CIITA genes in a T cell. In certain embodiments, a presently disclosed method comprises knocking out and/or knocking down the TRAC and TRBC genes in a T cell. In certain embodiments, a presently disclosed method comprises knocking out and/or knocking down the CIITA and TRBC genes in a T cell. In certain embodiments, a presently disclosed method comprises knocking out and/or knocking down the B2M, TRAC and CIITA genes in a T cell. In certain embodiments, a presently disclosed method comprises knocking out and/or knocking down the B2M, TRAC and TRBC genes in a T cell. In certain embodiments, a presently disclosed method comprises knocking out and/or knocking down the B2M, CIITA and TRBC genes in a T cell. In certain embodiments, a presently disclosed method comprises knocking out and/or knocking down the TRAC, CIITA and TRBC genes in a T cell. In certain embodiments, a presently disclosed method comprises knocking out and/or knocking down the B2M, TRAC, CIITA and TRBC genes in a T cell.

In certain embodiments, the knocking out and/or knocking down of one or more genes, two or more genes, three or more genes or four or more genes selected from the group consisting of B2M, TRAC, CIITA and TRBC in a T cell can: (1) prevent GvH response; (2) prevent HvG response; and/or (3) improve T cell safety and efficacy. For example, but not by way of limitation, the knocking out and/or knocking down of one or more genes selected from the group consisting of B2M, TRAC, CIITA and TRBC in a T cell can be used to generate an “allogeneic” cell, e.g., an allogeneic T cell. In certain embodiments, the knocking out and/or knocking down of one or more genes selected from the group consisting of B2M, TRAC, CIITA and TRBC can be employed in “allogeneic” cell therapies, in which cells are harvested from a subject, modified to knock-out or knock-down, e.g., disrupt, B2M, TRAC, CIITA and/or TRBC expression, and then returned to a different subject.

In certain embodiments, the knocking out and/or knocking down of one or more genes, two or more genes, three or more genes or four or more genes selected from the group consisting of B2M, TRAC, CIITA and TRBC in a T cell results in the reduction of MHC II receptor expression in the T cell as compared to a T cell that is not modified. In certain embodiments, a population of cells that has been modified to knockout and/or knockdown one or more genes selected from the group consisting of B2M, TRAC, CIITA and TRBC exhibits a reduction in MHC II receptor, TCR or B2M expression of at least about 10%, at least about 20%, at least about 30%, at least about 40%, at least about 50%, at least about 60%, at least about 70%, at least about 80% or at least about 90% relative to the amount of MHC II receptor, TCR or B2M expression in a population of cells that have not been modified.

In certain embodiments, the knocking out and/or knocking down of more than one gene can involve the use of different nucleases for the editing of each target gene. For example, but not by way of limitation, a CRISPR/Cpf1 editing system can be used to knock out and/or knock down one target gene and a CRISPR/Cas9 editing system can be used to knock out and/or knock down a second target gene.

The present disclosure provides an isolated CRISPR/Cpf1-edited T cell or a population of CRISPR/Cpf1-edited T cells that include one or more modifications in one or more endogenous genes of a T cell disclosed herein. In certain embodiments, the CRISPR/Cpf1-edited T cell or population of CRISPR/Cpf1-edited T cells include one or more components of a CRISPR/Cpf1 editing system. Alternatively, the CRISPR/Cpf1-edited T cell or population of CRISPR/Cpf1-edited T cells do not include one or more components of a CRISPR/Cpf1 editing system. In certain embodiments, less than about 10%, less than about 9%, less than about 8%, less than about 7%, less than about 6%, less than about 5%, less than about 4%, less than about 3%, less than about 2% or less than about 1% of the cells in the population of CRISPR/Cpf1-edited cells include one or more components of a CRISPR/Cpf1 editing system.

In certain embodiments, the T cell is a CD8⁺ T cell, a CD8⁺ naïve T cell, a CD4⁺ central memory T cell, a CD8⁺ central memory T cell, a CD4⁺ effector memory T cell, a CD4⁺ effector memory T cell, a CD4⁺ T cell, a CD4⁺ stem cell memory T cell, a CD8⁺ stem cell memory T cell, a CD4⁺ helper T cell, a regulatory T cell, a cytotoxic T cell, a natural killer T cell, a CD4+ naïve T cell, a TH17 CD4⁺ T cell, a TH1 CD4⁺ T cell, a TH2 CD4⁺ T cell, a TH9 CD4⁺ T cell, a CD4⁺ Foxp3⁺ T cell, a CD4⁺ CD25⁺ CD127⁻ T cell or a CD4⁺ CD25⁺ CD127⁻ Foxp3⁺ T cell.

In certain embodiments, the instant disclosure relates to the use of CRISPR/Cpf1-mediated editing of an endogenous gene of a T cell selected from the group consisting of FAS, BID, CTLA4, PDCD1, CBLB, PTPN6, B2M, TRAC, CIITA, TRBC and any combination thereof. For example, but not by way of limitation, the modification is generated by the delivery of one or more complexes comprising a Cpf1 RNA-guided nuclease and a gRNA molecule, e.g., RNP complexes, that targets a portion of a FAS gene sequence, a portion of a BID gene sequence, a portion of a CTLA4 gene sequence, a portion of a PDCD1 gene sequence, a portion of a CBLB gene sequence, a portion of a PTPN6 gene sequence, a portion of a B2M gene sequence, a portion of a TRAC gene sequence, a portion of a CIITA gene sequence, a portion of a TRBC gene sequence or a combination thereof. In certain embodiments, two or more, three or more, four or more, five or more, six or more, seven or more, eight or more, nine or more or ten complexes, e.g., RNP complexes, can be delivered, where each of the complexes target a different gene. In certain embodiments, at least about 5%, at least about 10%, at least about 20%, at least about 30%, at least about 40%, at least about 50%, at least about 60%, at least about 70%, at least about 80% or at least about 90% of the cells in the population of T cells are edited and/or modified. In certain embodiments, at least about 5%, at least about 10%, at least about 20%, at least about 30%, at least about 40%, at least about 50%, at least about 60%, at least about 70%, at least about 80% or at least about 90% of the cells in the population of T cells have a productive indel, e.g., in at least one of the endogenous T cell genes selected from the group consisting of FAS, BID, CTLA4, PDCD1, CBLB, PTPN6, B2M, TRAC, CIITA and TRBC.

Benchmarking Assays for Cpf1 Variants, Distinct Cell Types, and Formulations

CRISPR/Cpf1-mediated editing of a target nucleic acid sequence and/or modulation of expression of a target nucleic acid sequence can be evaluated by comparing the activity of a test CRISPR/Cpf1 editing system to a control CRISPR/RNA-guided nuclease editing system with respect to a target nucleic acid sequence, e.g., a “matched site” target nucleic acid sequence.

A matched site target nucleic acid sequence incorporates both the requirements to be edited by Cpf1 as well as a second RNA-guided nuclease, e.g., Cas9. For example, the TTTV AsCpf1 wild type protospacer adjacent motif (“PAM”) and a NGG SpCas9 wild type PAM can be employed in the instant example. As noted above, the test Cpf1 protein can comprise one or more modifications relative to the wild type Cpf1 protein. Examples of such modifications include, but are not limited to, the aforementioned modifications to incorporate one or more NLS sequence, to incorporate a six-histidine purification sequence, and the alteration of a Cpf1 protein cysteine amino acid, as well as combinations thereof.

Exemplary matched site target nucleic acid sequences that can be employed in the instant example include Matched Site 1 (“MS1”; SEQ ID NO: 13), Matched Site 5 (“MS5”; SEQ ID NO: 14), Matched Site 11 (“MS11”; SEQ ID NO: 15), and Matched Site 18 (“MS18”; SEQ ID NO: 16).

To evaluate CRISPR/Cpf1-mediated versus CRISPR/Cas9-mediated editing of a target nucleic acid sequence and/or modulation of expression of a target nucleic acid sequence in a particular cell type, e.g. CD34⁺ HSCs, a CRISPR/Cpf1 genome editing system, i.e., a system comprising a Cpf1 RNA-guided nuclease and a gRNA complementary to at least a portion of a target nucleic acid comprising a matched site target, is introduced, e.g., as an RNP or via the use of a vector coding for the components of the system, into the cell of the cell type of interest. The editing of the target nucleic acid sequence and/or modulation of expression of a target nucleic acid sequence can be detected as disclosed herein. The detected editing of the target nucleic acid sequence and/or modulation of expression of a target nucleic acid sequence can then be compared to the editing of the target nucleic acid sequence and/or modulation of expression of a target nucleic acid sequence detected when a CRISPR/Cas9 genome editing system is employed with the same matched site target and the same cell type.

The above-described method of comparing CRISPR/Cpf1-mediated versus CRISPR/Cas9-mediated editing (or editing by another CRISPR-based system) of a target nucleic acid sequence and/or modulation of expression of a target nucleic acid sequence allows for an evaluation of particular attributes of the CRISPR/Cpf1-mediated editing system employed. For example, but not by way of limitation, such methods can be used to evaluate CRISPR/Cpf1-mediated versus CRISPR/Cas9-mediated editing of a target nucleic acid sequence and/or modulation of expression of a target nucleic acid sequence to identify differences in activity of Cpf1 RNA-guided nucleases and/or gRNAs prepared by distinct manufacturing process. Such methods can also identify differences in activity of Cpf1 RNA-guided nucleases and/or gRNAs present in distinct formulations as well as those employing distinct delivery strategies.

In certain embodiments, the present disclosure relates to assays for the comparison of CRISPR/Cpf1-mediated editing of a target nucleic acid sequence and/or modulation of expression of a target nucleic acid sequence by a test CRISPR/Cpf1 genome editing system to a control RNA-guided nuclease genome editing system. More specifically, the present disclosure provides assays which employ a matched site (e.g., a cell containing a matched site 5) to which a gene editing system is targeted (e.g., CRISPR/Cas9 or CRISPR/Cpf1 or a variant thereof with a gRNA that is complementary to the matched site), such that the level or efficiency of editing at the matched site is an indication of how efficient the gene editing system will be in editing at any other site. In other words, the various components of the gene editing system can be varied and evaluated for editing efficiency by measuring the level or efficiency of editing that is achieved at a matched site (e.g., matched site 5).

For example, but not by way of limitation, the test and control gene or genome editing systems can differ by any one or more of the following aspects: the sequence of the RNA-guided nuclease; the source, e.g., method of manufacture, of a component of a genome editing system; the formulation of one or more component of the genome editing system; and the identity of the cell into which the genome editing system is introduced, e.g., cell type or method of preparation of the cell. In certain embodiments, the assays described herein allow for quality control analysis of test genome editing systems. In certain embodiments, the assays of the present disclosure will assess CRISPR/Cpf1-mediated editing of a target nucleic acid sequence and/or modulation of expression of a target nucleic acid sequence wherein the target comprises a matched site sequence.

Electroporation Pulse Code Screening

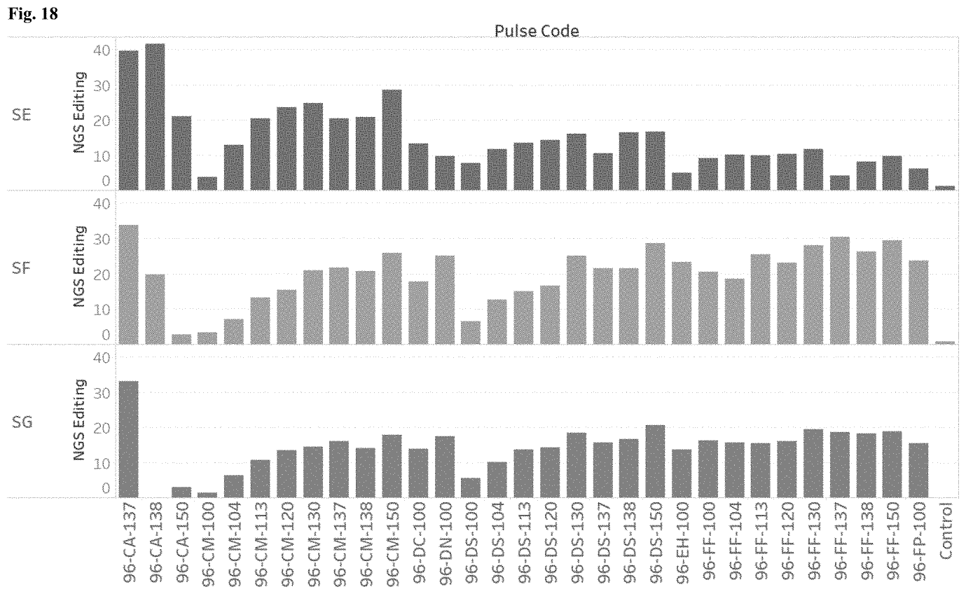

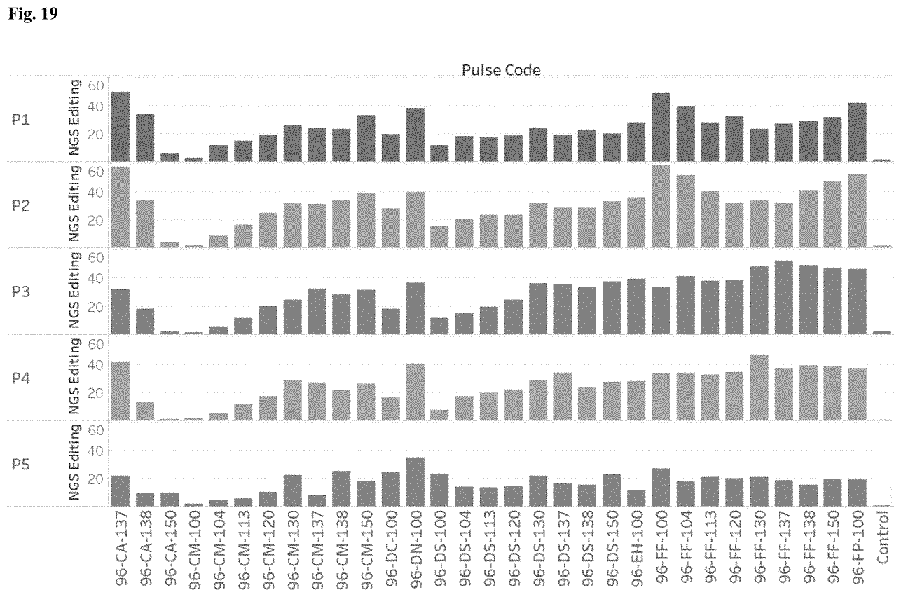

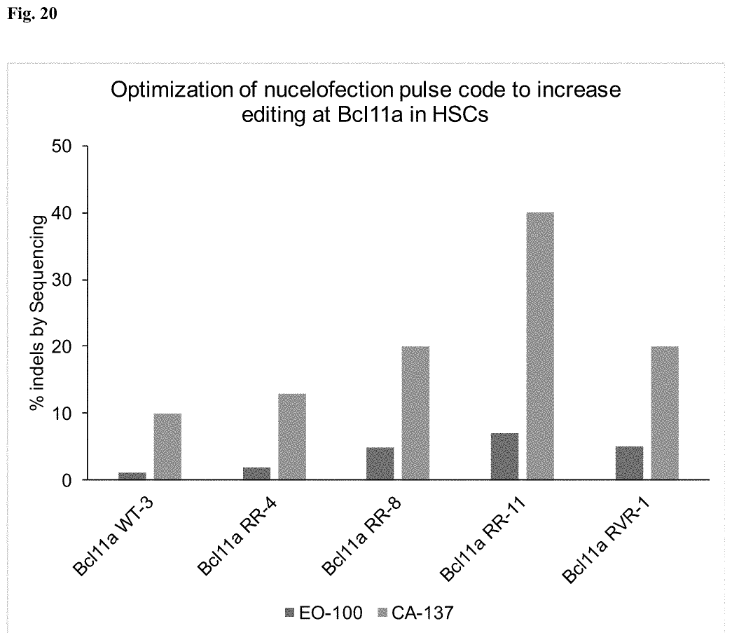

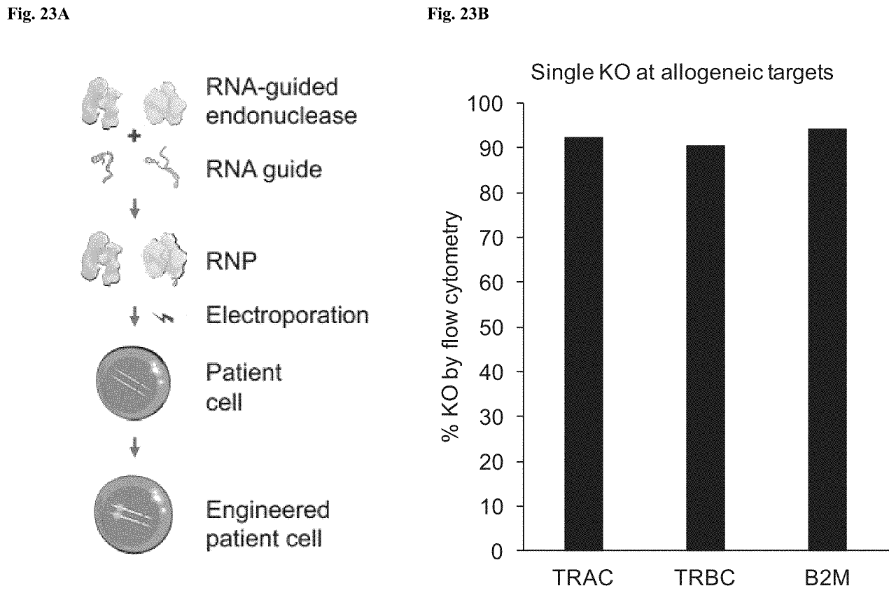

The present disclosure further provides electroporation pulse codes that result in higher editing at target sites. As shown in the examples, the screening of electroporation pulse codes allows for the identification of codes leading to higher efficiency editing of by the Cpf1 RNA-guided nucleases of the present disclosure. For example, but not by way of limitation, FIG. 18 depicts nucleofection screening for AsCpf1 in HUDEPs using a series of specific pulse codes and solutions. Similarly, FIG. 19 depicts an exemplary nucleofection screening for AsCpf1 in HSCs. In certain embodiments, the pulse codes CA-137 and CA-138 can be used to facilitate higher efficiency editing by Cpf1 RNA-guided nucleases. For example, but not by way of limitation, FIGS. 20 and 23C confirm the increased efficiency of the CA-137 pulse code.

### Methods of Treatment

The present disclosure further provides methods of treating diseases and/or disorders by administering cells that have been edited using the genome editing methods disclosed. In certain embodiments, the present disclosure relates to methods of treating a subject by modifying one or more cells of the subject. In certain embodiments, the one or more cells are modified ex vivo and then administered to the subject. For example, but not by way of limitation, methods for treating a subject can include contacting a cell from the subject, e.g., ex vivo, with (a) a gRNA molecule complementary to a target sequence of a target nucleic acid; and (b) a Cpf1 RNA-guided nuclease disclosed herein. In certain embodiments, the present disclosure provides methods for treating a subject that includes administering to the subject one or more cells modified by a CRISPR/Cpf1 system of the present disclosure. In certain embodiments, the one or more cells are obtained from a donor, genetically modified using a CRISPR/Cpf1 system of the present disclosure and then administered to a subject.

In certain embodiments, methods of the present disclosure can include administering to a subject in need thereof T cells that have been edited using the using the genome editing methods disclosed, e.g., to produce allogeneic T cells. For example, but not by way of limitation, a method of the present disclosure can include the administration of one or more T cells that have been edited to knock-out or knock-down FAS, BID, CTLA4, PDCD1, CBLB, PTPN6, B2M, TRAC, CIITA and/or TRBC expression. In certain embodiments, the T cells have been edited to knock-out or knock-down B2M, TRAC, CIITA and/or TRBC expression. In certain embodiments, the one or more T cells have been edited ex vivo and then administered to the subject. In certain embodiments, the one or more cells are obtained from a donor. In certain embodiments, such T cells can be used to treat a subject that has cancer or an autoimmune disorder. In certain embodiments, in the population of CRISPR/Cpf1-edited T cells that are administered to the subject, less than about 10%, less than about 9%, less than about 8%, less than about 7%, less than about 6%, less than about 5%, less than about 4%, less than about 3%, less than about 2% or less than about 1% of the cells in the population of CRISPR/Cpf1-edited cells include one or more components of a CRISPR/Cpf1 editing system.

In certain embodiments, methods of the present disclosure can include administering to a subject in need thereof CD34+ hematopoietic stem and progenitor cells (HSPCs) that have been edited using the using the genome editing methods disclosed. In certain embodiments, the CD34+ cells can be edited to knock-out or knock-down BCL11a or HBG expression. For example, but not by way of limitation, CD34+ hematopoietic stem and progenitor cells (HSPCs) that have been edited using the genome editing methods disclosed herein may be used for the treatment of a hemoglobinopathy in a subject in need thereof. In certain embodiments, the hemoglobinopathy may be severe sickle cell disease (SCD) or thalassemia, such as β-thalassemia, δ-thalassemia or β/δ-thalassemia. In certain embodiments, an exemplary protocol for treatment of a hemoglobinopathy may include harvesting CD34+ HSPCs from a subject in need thereof, ex vivo editing of the autologous CD34+ HSPCs using the genome editing methods disclosed herein, followed by reinfusion of the edited autologous CD34+ HSPCs into the subject. In certain embodiments, treatment with edited autologous CD34+ HSPCs may result in increased HbF induction.

Prior to harvesting CD34+ HSPCs, in certain embodiments, a subject may discontinue treatment with hydroxyurea, if applicable, and receive blood transfusions to maintain sufficient hemoglobin (Hb) levels. In certain embodiments, a subject may be administered intravenous plerixafor (e.g., 0.24 mg/kg) to mobilize CD34+ HSPCs from bone marrow into peripheral blood. In certain embodiments, a subject may undergo one or more leukapheresis cycles (e.g., approximately one month between cycles, with one cycle defined as two plerixafor-mobilized leukapheresis collections performed on consecutive days). In certain embodiments, the number of leukapheresis cycles performed for a subject may be the number required to achieve a dose of edited autologous CD34+ HSPCs (e.g., ≥2×10⁶ cells/kg, ≥3×10⁶ cells/kg, ≥4×10⁶ cells/kg, ≥5×10⁶ cells/kg, 2×10⁶ cells/kg to 3×10⁶ cells/kg, 3×10⁶ cells/kg to 4×10⁶ cells/kg, 4×10⁶ cells/kg to 5×10⁶ cells/kg) to be reinfused back into the subject, along with a dose of unedited autologous CD34+ HSPCs/kg for backup storage (e.g., ≥1.5×10⁶ cells/kg). In certain embodiments, the CD34+ HSPCs harvested from the subject may be edited using any of the genome editing methods discussed herein. In certain embodiments, any one or more of the gRNAs and one or more of the RNA-guided nucleases disclosed herein may be used in the genome editing methods.

In certain embodiments, the treatment may include an autologous stem cell transplant. In certain embodiments, a subject may undergo myeloablative conditioning with busulfan conditioning (e.g., dose-adjusted based on first-dose pharmacokinetic analysis, with a test dose of 1 mg/kg). In certain embodiments, conditioning may occur for four consecutive days. In certain embodiments, following a three-day busulfan washout period, edited autologous CD34+ HSPCs (e.g., ≥2×10⁶ cells/kg, ≥3×10⁶ cells/kg, ≥4×10⁶ cells/kg, ≥5×10⁶ cells/kg, 2×10⁶ cells/kg to 3×10⁶ cells/kg, 3×10⁶ cells/kg to 4×10⁶ cells/kg, 4×10⁶ cells/kg to 5×10⁶ cells/kg) may be reinfused into the subject (e.g., into peripheral blood). In certain embodiments, the edited autologous CD34+ HSPCs may be manufactured and cryopreserved for a particular subject. In certain embodiments, a subject may attain neutrophil engraftment following a sequential myeloablative conditioning regimen and infusion of edited autologous CD34+ cells. Neutrophil engraftment may be defined as three consecutive measurements of ANC≥0.5×10⁹/L. In certain embodiments, in the population of CRISPR/Cpf1-edited CD34+ HSPCs that are administered to the subject, less than about 10%, less than about 9%, less than about 8%, less than about 7%, less than about 6%, less than about 5%, less than about 4%, less than about 3%, less than about 2% or less than about 1% of the cells in the population of CRISPR/Cpf1-edited CD34+ HSPCs include one or more components of a CRISPR/Cpf1 editing system.

In certain embodiments, the CRISPR/Cpf1-mediated editing systems of the present disclosure can result in clinically relevant or therapeutically relevant editing efficiencies of about 10% or more. For example, but not by way of limitation, CRISPR/Cpf1-mediated editing systems of the present disclosure can result in clinically relevant or therapeutically relevant editing efficiencies of about 5% or more, about 10 or more, 15% or more, of about 20% or more, of about 25% or more, of about 30% or more, of about 35% or more, of about 40% or more, of about 45% or more, of about 50% or more, of about 55% or more, of about 60% or more, of about 65% or more, of about 70% or more, of about 75% or more, of about 80% or more, of about 85% or more, of about 90% or more, of about 95% or more, of about 96% or more, of about 97% or more, of about 98% or more or of about 99% or more.

In certain embodiments, at least about 5%, at least about 10%, at least about 20%, at least about 30%, at least about 40%, at least about 50%, at least about 60%, at least about 70%, at least about 80% or at least about 90% of the cells in the population of cells that are to be administered in a method of treatment disclosed herein are modified.

In certain embodiments, less than about 10%, less than about 5%, less than about 1%, less than about 0.5%, less than about 0.25% or less than about 0.1% of the cells in the population of CRISPR/Cpf1-edited cells include one or more components of a CRISPR/Cpf1 editing system.

### Genome Editing Systems

The term “genome editing system” or “gene editing system” refers to any system having RNA-guided DNA editing activity. Genome editing systems of the present disclosure include at least two components adapted from naturally occurring CRISPR systems: a guide RNA (gRNA) and an RNA-guided nuclease. These two components form a complex that is capable of associating with a specific nucleic acid sequence and editing the DNA in or around that nucleic acid sequence, for instance by making one or more of a single-strand break (an SSB or nick), a double-strand break (a DSB) and/or a point mutation.

Naturally occurring CRISPR systems are organized evolutionarily into two classes and five types (Makarova et al. Nat Rev Microbiol. 2011 June; 9(6): 467-477 (Makarova), incorporated by reference herein), and while genome editing systems of the present disclosure may adapt components of any type or class of naturally occurring CRISPR system, the embodiments presented herein are generally adapted from Class 2, and type II or V CRISPR systems. Class 2 systems, which encompass types II and V, are characterized by relatively large, multidomain RNA-guided nuclease proteins (e.g., Cas9 or Cpf1) and one or more guide RNAs (e.g., a crRNA and, optionally, a tracrRNA) that form ribonucleoprotein (RNP) complexes that associate with (i.e., target) and cleave specific loci complementary to a targeting (or spacer) sequence of the crRNA. Genome editing systems according to the present disclosure similarly target and edit cellular DNA sequences, but differ significantly from CRISPR systems occurring in nature. For example, the unimolecular guide RNAs described herein do not occur in nature, and both guide RNAs and RNA-guided nucleases according to this disclosure may incorporate any number of non-naturally occurring modifications.

Genome editing systems can be implemented (e.g., administered or delivered to a cell or a subject) in a variety of ways, and different implementations may be suitable for distinct applications. For instance, a genome editing system is implemented, in certain embodiments, as a protein/RNA complex (a ribonucleoprotein, or RNP), which can be included in a pharmaceutical composition that optionally includes a pharmaceutically acceptable carrier and/or an encapsulating agent, such as a lipid or polymer micro- or nano-particle, micelle, liposome, etc. In certain embodiments, a genome editing system is implemented as one or more nucleic acids encoding the RNA-guided nuclease and guide RNA components described above (optionally with one or more additional components); in certain embodiments, the genome editing system is implemented as one or more vectors comprising such nucleic acids, for instance a viral vector such as an adeno-associated virus; and in certain embodiments, the genome editing system is implemented as a combination of any of the foregoing. Additional or modified implementations that operate according to the principles set forth herein will be apparent to the skilled artisan and are within the scope of this disclosure.

It should be noted that the genome editing systems of the present disclosure can be targeted to a single specific nucleotide sequence, or may be targeted to—and capable of editing in parallel—two or more specific nucleotide sequences through the use of two or more guide RNAs. The use of multiple gRNAs is referred to as “multiplexing” throughout this disclosure, and can be employed to target multiple, unrelated target sequences of interest, or to form multiple SSBs or DSBs within a single target domain and, in some cases, to generate specific edits within such target domain. For example, International Patent Publication No. WO 2015/138510 by Maeder et al. (Maeder), which is incorporated by reference herein, describes a genome editing system for correcting a point mutation (C.2991+1655A to G) in the human CEP290 gene that results in the creation of a cryptic splice site, which in turn reduces or eliminates the function of the gene. The genome editing system of Maeder utilizes two guide RNAs targeted to sequences on either side of (i.e., flanking) the point mutation, and forms DSBs that flank the mutation. This, in turn, promotes deletion of the intervening sequence, including the mutation, thereby eliminating the cryptic splice site and restoring normal gene function.

As another example, WO 2016/073990 by Cotta-Ramusino et al. (“Cotta-Ramusino et al.”), incorporated by reference herein in its entirety, describes a genome editing system that utilizes two gRNAs in combination with a Cas9 nickase (a Cas9 that makes a single strand nick such as S. pyogenes D10A), an arrangement termed a “dual-nickase system.” The dual-nickase system of Cotta-Ramusino et al. is configured to make two nicks on opposite strands of a sequence of interest that are offset by one or more nucleotides, which nicks combine to create a double strand break having an overhang (5′ in the case of Cotta-Ramusino et al., though 3′ overhangs are also possible). The overhang, in turn, can facilitate homology directed repair events in some circumstances. And, as another example, WO 2015/070083 by Palestrant et al. (“Palestrant”, incorporated by reference herein in its entirety) describes a gRNA targeted to a nucleotide sequence encoding Cas9 (referred to as a “governing RNA”), which can be included in a genome editing system comprising one or more additional gRNAs to permit transient expression of a Cas9 that might otherwise be constitutively expressed, for example in some virally transduced cells. These multiplexing applications are intended to be exemplary, rather than limiting, and the skilled artisan will appreciate that other applications of multiplexing are generally compatible with the genome editing systems described here.

Genome editing systems can, in some instances, form double strand breaks that are repaired by cellular DNA double-strand break mechanisms such as NHEJ or HDR. These mechanisms are described throughout the literature, for example by Davis & Maizels, PNAS, 111(10):E924-932, Mar. 11, 2014 (Davis) (describing Alt-HDR); Frit et al. DNA Repair 17(2014) 81-97 (Frit) (describing Alt-NHEJ); and Iyama and Wilson III, DNA Repair (Amst.) 2013-August; 12(8): 620-636 (Iyama) (describing canonical HDR and NHEJ pathways generally).

Where genome editing systems operate by forming DSBs, such systems optionally include one or more components that promote or facilitate a particular mode of double-strand break repair or a particular repair outcome. For instance, Cotta-Ramusino et al. also describes genome editing systems in which a single stranded oligonucleotide “donor template” is added; the donor template is incorporated into a target region of cellular DNA that is cleaved by the genome editing system, and can result in a change in the target sequence.

In certain embodiments, genome editing systems modify a target sequence, or modify expression of a gene in or near the target sequence, without causing single- or double-strand breaks. For example, a genome editing system may include an RNA-guided nuclease fused to a functional domain that acts on DNA, thereby modifying the target sequence or its expression. As one example, an RNA-guided nuclease can be connected to (e.g., fused to) a cytidine deaminase functional domain, and may operate by generating targeted C-to-A substitutions. Exemplary nuclease/deaminase fusions are described in Komor et al. Nature 533, 420-424 (19 May 2016) (“Komor”), which is incorporated by reference. Alternatively, a genome editing system may utilize a cleavage-inactivated (i.e., a “dead”) nuclease, such as a dead Cas9 (dCas9), and may operate by forming stable complexes on one or more targeted regions of cellular DNA, thereby interfering with functions involving the targeted region(s) including, without limitation, mRNA transcription, chromatin remodeling, etc.

In certain embodiments, the genome editing systems encompassed by the present disclosure will exhibit certain minimal percentages of editing in standard assays. For example, but not by way of limitation, certain genome editing systems encompassed by the present disclosure will exhibit at least 10%, 20%, 30%, 40%, 50%, 60%, 70%, 80%, 90% or 95% editing in certain standard assays. One or more assays known in the art or those described herein, such as, for example, those described in Example 1 below, can be used for assessing CRISPR/Cpf1 mediated editing of a target nucleic acid sequence. As an example, Example 1, below, describes the evaluation of CRISPR/Cpf1-mediated versus CRISPR/Cas9-mediated editing of a target nucleic acid sequence and/or modulation of expression of a target nucleic acid sequence in a particular cell type, e.g. CD34⁺ HSCs, a CRISPR/Cpf1 genome editing system, i.e., a system comprising a Cpf1 RNA-guided nuclease and a gRNA complementary to at least a portion of a target nucleic acid comprising a matched site target, is introduced, e.g., as an RNP or via the use of a vector coding for the components of the system, into the cell of the cell type of interest. The editing of the target nucleic acid sequence and/or modulation of expression of a target nucleic acid sequence is detected as disclosed herein. The detected editing of the target nucleic acid sequence and/or modulation of expression of a target nucleic acid sequence is compared to the editing of the target nucleic acid sequence and/or modulation of expression of a target nucleic acid sequence detected when a CRISPR/Cas9 genome editing system is employed with the same matched site target and the same cell type.

In certain embodiments, a genome editing system of the present disclosure can knock out or knockdown one or more, two or more, three or more or four or more genes selected from the group consisting of B2M, TRAC, CIITA and TRBC simultaneously in a cell population. In certain embodiments, a genome editing system of the present disclosure can comprise one or more, two or more, three or more or four or more gRNA molecules, where each gRNA molecule comprises a targeting domain for a different gene, e.g., a gene selected from the B2M, TRAC, CIITA and TRBC genes. For example, but not by way of limitation, a multiplex genome editing system of the present disclosure can include (i) a first RNP complex comprising a first guide RNA (gRNA) comprising a first targeting domain that is complementary to a target sequence of first gene and a first Cpf1 RNA-guided nuclease, (ii) a second RNP complex comprising a second gRNA molecule comprising a second targeting domain that is complementary to a target sequence of a second gene and a second Cpf1 RNA-guided nuclease, (iii) a third RNP complex comprising a third gRNA molecule comprising a third targeting domain that is complementary to a target sequence of a third gene and a fourth Cpf1 RNA-guided nuclease and/or (iv) a fourth RNP complex comprising a fourth gRNA molecule comprising a fourth targeting domain that is complementary to a target sequence of a fourth gene and a fourth Cpf1 RNA-guided nuclease. In certain embodiments, the first gene, the second gene, the third gene and the fourth gene are selected from the group consisting of B2M, TRAC, CIITA and TRBC. In certain embodiments, a targeting domain of a gRNA molecule for targeting B2M comprises a targeting domain sequence listed in Tables 6, 7 and 8. In certain embodiments, a targeting domain of a gRNA molecule for targeting TRAC comprises a targeting domain sequence listed in Tables 2 and 3. In certain embodiments, a targeting domain of a gRNA molecule for targeting CIITA comprises a targeting domain sequence listed in Table 9. In certain embodiments, a targeting domain of a gRNA molecule for targeting TRBC comprises a targeting domain sequence listed in Tables 4 and 5. In certain embodiments, the editing efficiency can be >80%, >85%, >90%, >95%, >98% or >99% for all target genes. In certain embodiments, the cell population can be a T cell population.

Guide RNA (gRNA) Molecules

The terms “guide RNA” and “gRNA” refer to any nucleic acid that promotes the specific association (or “targeting”) of an RNA-guided nuclease such as Cpf1 to a target sequence such as a genomic or episomal sequence in a cell. gRNAs can be unimolecular (comprising a single RNA molecule, and referred to alternatively as chimeric), or modular (comprising more than one, and typically two, separate RNA molecules, such as a crRNA and a tracrRNA, which are usually associated with one another, for instance by duplexing). gRNAs and their component parts are described throughout the literature, for instance in Briner et al. (Molecular Cell 56(2), 333-339, Oct. 23, 2014 (Briner), which is incorporated by reference), and in Cotta-Ramusino.

In bacteria and archaea, type II CRISPR systems generally comprise an RNA-guided nuclease protein such as Cas9, a CRISPR RNA (crRNA) that includes a 5′ region that is complementary to a foreign sequence, and a trans-activating crRNA (tracrRNA) that includes a 5′ region that is complementary to, and forms a duplex with, a 3′ region of the crRNA. While not intending to be bound by any theory, it is thought that this duplex facilitates the formation of—and is necessary for the activity of—the Cas9/gRNA complex. As type II CRISPR systems were adapted for use in gene editing, it was discovered that the crRNA and tracrRNA could be joined into a single unimolecular or chimeric guide RNA, in one non-limiting example, by means of a four nucleotide (e.g., GAAA) “tetraloop” or “linker” sequence bridging complementary regions of the crRNA (at its 3′ end) and the tracrRNA (at its 5′ end). (Mali et al. Science. 2013 Feb. 15; 339(6121): 823-826 (“Mali”); Jiang et al. Nat Biotechnol. 2013 March; 31(3): 233-239 (“Jiang”); and Jinek et al., 2012 Science August 17; 337(6096): 816-821 (“Jinek”), all of which are incorporated by reference herein.)

Guide RNAs, whether unimolecular or modular, include a “targeting domain” that is fully or partially complementary to a target domain within a target sequence, such as a DNA sequence in the genome of a cell where editing is desired. Targeting domains are referred to by various names in the literature, including without limitation “guide sequences” (Hsu et al., Nat Biotechnol. 2013 September; 31(9): 827-832, (“Hsu”), incorporated by reference herein), “complementarity regions” (Cotta-Ramusino et al.), “spacers” (Briner) and generically as “crRNAs” (Jiang). Irrespective of the names they are given, targeting domains are typically 10-30 nucleotides in length, and in certain embodiments are 16-24 nucleotides in length (for instance, 16, 17, 18, 19, 20, 21, 22, 23 or 24 nucleotides in length), and are at or near the 5′ terminus of in the case of a Cas9 gRNA, and at or near the 3′ terminus in the case of a Cpf1 gRNA.

In addition to the targeting domains, gRNAs typically (but not necessarily, as discussed below) include a plurality of domains that may influence the formation or activity of gRNA/Cas9 and gRNA/Cpf1 complexes. For instance, as mentioned above, the duplexed structure formed by first and secondary complementarity domains of a gRNA (also referred to as a repeat:anti-repeat duplex) interacts with the recognition (REC) lobe of Cas9 and can mediate the formation of Cas9/gRNA complexes. (Nishimasu et al., Cell 156, 935-949, Feb. 27, 2014 (Nishimasu 2014) and Nishimasu et al., Cell 162, 1113-1126, Aug. 27, 2015 (Nishimasu 2015), both incorporated by reference herein). It should be noted that the first and/or second complementarity domains may contain one or more poly-A tracts, which can be recognized by RNA polymerases as a termination signal. The sequence of the first and second complementarity domains are, therefore, optionally modified to eliminate these tracts and promote the complete in vitro transcription of gRNAs, for instance through the use of A-G swaps as described in Briner, or A-U swaps. These and other similar modifications to the first and second complementarity domains are within the scope of the present disclosure.

Along with the first and second complementarity domains, Cas9 gRNAs typically include two or more additional duplexed regions that are involved in nuclease activity in vivo but not necessarily in vitro. (Nishimasu 2015). A first stem-loop one near the 3′ portion of the second complementarity domain is referred to variously as the “proximal domain,” (Cotta-Ramusino) “stem loop 1” (Nishimasu 2014 and 2015) and the “nexus” (Briner). One or more additional stem loop structures are generally present near the 3′ end of the gRNA, with the number varying by species: S. pyogenes gRNAs typically include two 3′ stem loops (for a total of four stem loop structures including the repeat:anti-repeat duplex), while S. aureus and other species have only one (for a total of three stem loop structures). A description of conserved stem loop structures (and gRNA structures more generally) organized by species is provided in Briner.

While the foregoing description has focused on gRNAs for use with Cas9, it should be appreciated that other RNA-guided nucleases have been (or may in the future be) discovered or invented which utilize gRNAs that differ in some ways from those described to this point. For instance, Cpf1 (“CRISPR from Prevotella and Franciscella 1”) is a recently discovered RNA-guided nuclease that does not require a tracrRNA to function. (Zetsche et al., 2015, Cell 163, 759-771 Oct. 22, 2015 (Zetsche I), incorporated by reference herein). A gRNA for use in a Cpf1 genome editing system generally includes a targeting domain and a complementarity domain (alternately referred to as a “handle”). It should also be noted that, in gRNAs for use with Cpf1, the targeting domain is usually present at or near the 3′ end, rather than the 5′ end as described above in connection with Cas9 gRNAs (the handle is at or near the 5′ end of a Cpf1 gRNA).

Those of skill in the art will appreciate that, although structural differences may exist between gRNAs from different prokaryotic species, or between Cpf1 and Cas9 gRNAs, the principles by which gRNAs operate are generally consistent. Because of this consistency of operation, gRNAs can be defined, in broad terms, by their targeting domain sequences, and skilled artisans will appreciate that a given targeting domain sequence can be incorporated in any suitable gRNA, including a unimolecular or chimeric gRNA, or a gRNA that includes one or more chemical modifications and/or sequential modifications (substitutions, additional nucleotides, truncations, etc.). Thus, for economy of presentation in this disclosure, gRNAs may be described solely in terms of their targeting domain sequences.

More generally, skilled artisans will appreciate that some aspects of the present disclosure relate to systems, methods and compositions that can be implemented using multiple RNA-guided nucleases. For this reason, unless otherwise specified, the term gRNA should be understood to encompass any suitable gRNA that can be used with any RNA-guided nuclease, and not only those gRNAs that are compatible with a particular species of Cas9 or Cpf1. By way of illustration, the term gRNA can, in certain embodiments, include a gRNA for use with any RNA-guided nuclease occurring in a Class 2 CRISPR system, such as a type II or type V or CRISPR system, or an RNA-guided nuclease derived or adapted therefrom.

The present disclosure provides gRNA molecules that comprise the sequence of any one of the gRNAs provided in Tables 2-9 and 19, and compositions thereof. The present disclosure further provides compositions that include one or more gRNAs comprising a sequence of a gRNA set forth in Tables 2-9 and 19, and compositions thereof. The present disclosure provides gRNAs that target the chromosomal regions (e.g., genomic coordinates) provided in Table 18, and compositions thereof.

The present disclosure provides gRNAs that result in greater than about 10% editing at a target site, e.g., in a population of cells. For example, but not by way of limitation, gRNAs of the present disclosure result in greater than about 15% editing, greater than about 20% editing, greater than about 25% editing, greater than about 30% editing, greater than about 35% editing, greater than about 40% editing, greater than about 45% editing, greater than about 50% editing, greater than about 55% editing, greater than about 60% editing, greater than about 65% editing, greater than about 70% editing, greater than about 75% editing, greater than about 80% editing, greater than about 85% editing, greater than about 90% editing, greater than about 95% editing, greater than about 96% editing, greater than about 97% editing, greater than about 98% editing or greater than about 99% editing at a target site, e.g., in a population of cells.

gRNA Design

Methods for selection and validation of target sequences as well as off-target analyses have been described previously, e.g., in Mali; Hsu; Fu et al., 2014 Nat biotechnol 32(3): 279-84, Heigwer et al., 2014 Nat methods 11(2):122-3; Bae et al. (2014) Bioinformatics 30(10): 1473-5; and Xiao A et al. (2014) Bioinformatics 30(8): 1180-1182. Each of these references is incorporated by reference herein. As a non-limiting example, gRNA design may involve the use of a software tool to optimize the choice of potential target sequences corresponding to a user's target sequence, e.g., to minimize total off-target activity across the genome. While off-target activity is not limited to cleavage, the cleavage efficiency at each off-target sequence can be predicted, e.g., using an experimentally-derived weighting scheme. These and other guide selection methods are described in detail in Maeder and Cotta-Ramusino et al.

gRNA Modifications

The activity, stability, or other characteristics of gRNAs can be altered through the incorporation of certain modifications. As one example, transiently expressed or delivered nucleic acids can be prone to degradation by, e.g., cellular nucleases. Accordingly, the gRNAs described herein can contain one or more modified nucleosides or nucleotides which introduce stability toward nucleases. While not wishing to be bound by theory it is also believed that certain modified gRNAs described herein can exhibit a reduced innate immune response when introduced into cells. Those of skill in the art will be aware of certain cellular responses commonly observed in cells, e.g., mammalian cells, in response to exogenous nucleic acids, particularly those of viral or bacterial origin. Such responses, which can include induction of cytokine expression and release and cell death, may be reduced or eliminated altogether by the modifications presented herein.

Certain exemplary modifications discussed in this section can be included at any position within a gRNA sequence including, without limitation at or near the 5′ end (e.g., within 1-10, 1-5, or 1-2 nucleotides of the 5′ end) and/or at or near the 3′ end (e.g., within 1-10, 1-5, or 1-2 nucleotides of the 3′ end). In some cases, modifications are positioned within functional motifs, such as the repeat-anti-repeat duplex of a Cas9 gRNA, a stem loop structure of a Cas9 or Cpf1 gRNA, and/or a targeting domain of a gRNA.

As one example, the 5′ end of a gRNA can include a eukaryotic mRNA cap structure or cap analog (e.g., a G(5)ppp(5)G cap analog, a m7G(5)ppp(5)G cap analog, or a 3′-O-Me-m7G(5)ppp(5)G anti reverse cap analog (ARCA)), as shown below:

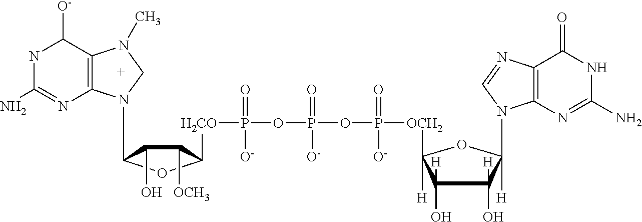

The cap or cap analog can be included during either chemical synthesis or in vitro transcription of the gRNA.

Along similar lines, the 5′ end of the gRNA can lack a 5′ triphosphate group. For instance, in vitro transcribed gRNAs can be phosphatase-treated (e.g., using calf intestinal alkaline phosphatase) to remove a 5′ triphosphate group.

Another common modification involves the addition, at the 3′ end of a gRNA, of a plurality (e.g., 1-10, 10-20, or 25-200) of adenine (A) residues referred to as a polyA tract. The polyA tract can be added to a gRNA during chemical synthesis, following in vitro transcription using a polyadenosine polymerase (e.g., E. coli Poly(A)Polymerase), or in vivo by means of a polyadenylation sequence, as described in Maeder.

It should be noted that the modifications described herein can be combined in any suitable manner, e.g. a gRNA, whether transcribed in vivo from a DNA vector, or in vitro transcribed gRNA, can include either or both of a 5′ cap structure or cap analog and a 3′ polyA tract.

Guide RNAs can be modified at a 3′ terminal U ribose. For example, the two terminal hydroxyl groups of the U ribose can be oxidized to aldehyde groups and a concomitant opening of the ribose ring to afford a modified nucleoside as shown below:

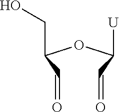

wherein “U” can be an unmodified or modified uridine.

The 3′ terminal U ribose can be modified with a 2′3′ cyclic phosphate as shown below:

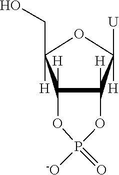

wherein “U” can be an unmodified or modified uridine.

Guide RNAs can contain 3′ nucleotides which can be stabilized against degradation, e.g., by incorporating one or more of the modified nucleotides described herein. In certain embodiments, uridines can be replaced with modified uridines, e.g., 5-(2-amino)propyl uridine, and 5-bromo uridine, or with any of the modified uridines described herein; adenosines and guanosines can be replaced with modified adenosines and guanosines, e.g., with modifications at the 8-position, e.g., 8-bromo guanosine, or with any of the modified adenosines or guanosines described herein.

In certain embodiments, sugar-modified ribonucleotides can be incorporated into the gRNA, e.g., wherein the 2′ OH-group is replaced by a group selected from H, —OR, —R (wherein R can be, e.g., alkyl, cycloalkyl, aryl, aralkyl, heteroaryl or sugar), halo, —SH, —SR (wherein R can be, e.g., alkyl, cycloalkyl, aryl, aralkyl, heteroaryl or sugar), amino (wherein amino can be, e.g., NH₂; alkylamino, dialkylamino, heterocyclyl, arylamino, diarylamino, heteroarylamino, diheteroarylamino, or amino acid); or cyano (—CN). In certain embodiments, the phosphate backbone can be modified as described herein, e.g., with a phosphothioate (PhTx) group. In certain embodiments, one or more of the nucleotides of the gRNA can each independently be a modified or unmodified nucleotide including, but not limited to 2′-sugar modified, such as, 2′-O-methyl, 2′-O-methoxyethyl, or 2′-Fluoro modified including, e.g., 2′-F or 2′-O-methyl, adenosine (A), 2′-F or 2′-O-methyl, cytidine (C), 2′-F or 2′-O-methyl, uridine (U), 2′-F or 2′-O-methyl, thymidine (T), 2′-F or 2′-O-methyl, guanosine (G), 2′-O-methoxyethyl-5-methyluridine (Teo), 2′-O-methoxyethyladenosine (Aeo), 2′-O-methoxyethyl-5-methylcytidine (m5Ceo), and any combinations thereof.

Guide RNAs can also include “locked” nucleic acids (LNA) in which the 2′ OH-group can be connected, e.g., by a C1-6 alkylene or C1-6 heteroalkylene bridge, to the 4′ carbon of the same ribose sugar. Any suitable moiety can be used to provide such bridges, include without limitation methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy or O(CH₂)ₙ-amino (wherein amino can be, e.g., NH₂; alkylamino, dialkylamino, heterocyclyl, arylamino, diarylamino, heteroarylamino, or diheteroarylamino, ethylenediamine, or polyamino).

In certain embodiments, a gRNA can include a modified nucleotide which is multicyclic (e.g., tricyclo; and “unlocked” forms, such as glycol nucleic acid (GNA) (e.g., R-GNA or S-GNA, where ribose is replaced by glycol units attached to phosphodiester bonds), or threose nucleic acid (TNA, where ribose is replaced with α-L-threofuranosyl-(3′→2′)).

Generally, gRNAs include the sugar group ribose, which is a 5-membered ring having an oxygen. Exemplary modified gRNAs can include, without limitation, replacement of the oxygen in ribose (e.g., with sulfur (S), selenium (Se), or alkylene, such as, e.g., methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for example, anhydrohexitol, altritol, mannitol, cyclohexanyl, cyclohexenyl, and morpholino that also has a phosphoramidate backbone). Although the majority of sugar analog alterations are localized to the 2′ position, other sites are amenable to modification, including the 4′ position. In certain embodiments, a gRNA comprises a 4′-S, 4′-Se or a 4′-C-aminomethyl-2′-O-Me modification.

In certain embodiments, deaza nucleotides, e.g., 7-deaza-adenosine, can be incorporated into the gRNA. In certain embodiments, O- and N-alkylated nucleotides, e.g., N6-methyl adenosine, can be incorporated into the gRNA. In certain embodiments, one or more or all of the nucleotides in a gRNA are deoxynucleotides.

In certain embodiments, the gRNA will comprise one or more linkers and/or processes of gRNA synthesis selected from those described in the international patent application having serial number PCT/US17/69019, which is incorporated by reference herein in its entirety.

### RNA-Guided Nucleases

RNA-guided nucleases according to the present disclosure include, but are not limited to, naturally-occurring Class 2 CRISPR nucleases such as Cpf1, as well as other nucleases derived or obtained therefrom, e.g., variants. RNA-guided nucleases can also be defined in functional terms. For example, RNA-guided nucleases are defined as those nucleases that: (a) interact with (e.g., complex with) a gRNA; and (b) together with the gRNA, associate with, and optionally cleave or modify, a target region of a DNA that includes (i) a sequence complementary to the targeting domain of the gRNA and, optionally, (ii) an additional sequence referred to as a “protospacer adjacent motif,” or “PAM,” which is described in greater detail below. As the following examples will illustrate, RNA-guided nucleases can be defined, in broad terms, by their PAM specificity and cleavage activity, even though variations may exist between individual RNA-guided nucleases that share the same PAM specificity or cleavage activity. Skilled artisans will appreciate that some aspects of the present disclosure relate to systems, methods and compositions that can be implemented using any suitable RNA-guided nuclease having a certain PAM specificity and/or cleavage activity. For this reason, unless otherwise specified, the term RNA-guided nuclease should be understood as a generic term, and not limited to any particular type (e.g., Cas9 vs. Cpf1), species (e.g., S. pyogenes vs. S. aureus) or variation (e.g., full-length vs. truncated or split; naturally-occurring PAM specificity vs. engineered PAM specificity, etc.) of RNA-guided nuclease.

The PAM sequence takes its name from its sequential relationship to the “protospacer” sequence that is complementary to gRNA targeting domains (or “spacers”). Together with protospacer sequences, PAM sequences define target regions or sequences for specific RNA-guided nuclease/gRNA combinations.

Various RNA-guided nucleases may require different sequential relationships between PAMs and protospacers. In general, Cas9s recognize PAM sequences that are 3′ of the protospacer. Cpf1, on the other hand, generally recognizes PAM sequences that are 5′ of the protospacer.

In addition to recognizing specific sequential orientations of PAMs and protospacers, RNA-guided nucleases can also recognize specific PAM sequences. S. aureus Cas9, for instance, recognizes a PAM sequence of NNGRRT or NNGRRV, wherein the N residues are immediately 3′ of the region recognized by the gRNA targeting domain. S. pyogenes Cas9 recognizes NGG PAM sequences. And F. novicida Cpf1 recognizes a TTN PAM sequence. PAM sequences have been identified for a variety of RNA-guided nucleases, and a strategy for identifying novel PAM sequences has been described by Shmakov et al., 2015, Molecular Cell 60, 385-397, Nov. 5, 2015. It should also be noted that engineered RNA-guided nucleases can have PAM specificities that differ from the PAM specificities of reference molecules (for instance, in the case of an engineered RNA-guided nuclease, the reference molecule may be the naturally occurring variant from which the RNA-guided nuclease is derived, or the naturally occurring variant having the greatest amino acid sequence homology to the engineered RNA-guided nuclease).

In addition to their PAM specificity, RNA-guided nucleases can be characterized by their DNA cleavage activity: naturally-occurring RNA-guided nucleases typically form DSBs in target nucleic acids, but engineered variants have been produced that generate only SSBs (discussed above) Ran & Hsu, et al., Cell 154(6), 1380-1389, Sep. 12, 2013 (Ran), incorporated by reference herein), or that that do not cut at all.

Cpf1

The crystal structure of Acidaminococcus sp. Cpf1 in complex with crRNA and a double-stranded (ds) DNA target including a TTTN PAM sequence has been solved by Yamano et al. (Cell. 2016 May 5; 165(4): 949-962 (Yamano), incorporated by reference herein). Cpf1, like Cas9, has two lobes: a REC (recognition) lobe, and a NUC (nuclease) lobe. The REC lobe includes REC1 and REC2 domains, which lack similarity to any known protein structures. The NUC lobe, meanwhile, includes three RuvC domains (RuvC-I, -II and -III) and a BH domain. However, in contrast to Cas9, the Cpf1 REC lobe lacks an HNH domain, and includes other domains that also lack similarity to known protein structures: a structurally unique PI domain, three Wedge (WED) domains (WED-I, -II and -III), and a nuclease (Nuc) domain.

While Cas9 and Cpf1 share similarities in structure and function, it should be appreciated that certain Cpf1 activities are mediated by structural domains that are not analogous to any Cas9 domains. For instance, cleavage of the complementary strand of the target DNA appears to be mediated by the Nuc domain, which differs sequentially and spatially from the HNH domain of Cas9. Additionally, the non-targeting portion of Cpf1 gRNA (the handle) adopts a pseudoknot structure, rather than a stem loop structure formed by the repeat:antirepeat duplex in Cas9 gRNAs.

Modifications of RNA-Guided Nucleases

The RNA-guided nucleases described above have activities and properties that can be useful in a variety of applications, but the skilled artisan will appreciate that RNA-guided nucleases can also be modified in certain instances, to alter cleavage activity, PAM specificity, or other structural or functional features.

Turning first to modifications that alter cleavage activity, mutations that reduce or eliminate the activity of domains within the NUC lobe have been described above. Exemplary mutations that may be made in the RuvC domains, in the Cas9 HNH domain, or in the Cpf1 Nuc domain are described in Ran and Yamano, as well as in Cotta-Ramusino. In general, mutations that reduce or eliminate activity in one of the two nuclease domains result in RNA-guided nucleases with nickase activity, but it should be noted that the type of nickase activity varies depending on which domain is inactivated. As one example, inactivation of a RuvC domain of a Cas9 will result in a nickase that cleaves the complementary or top strand. On the other hand, inactivation of a Cas9 HNH domain results in a nickase that cleaves the bottom or non-complementary strand.

Modifications of PAM specificity relative to naturally occurring Cas9 reference molecules has been described by Kleinstiver et al. for both S. pyogenes (Kleinstiver et al., Nature. 2015 Jul. 23; 523(7561):481-5 (Kleinstiver I) and S. aureus (Kleinstiver et al., Nat Biotechnol. 2015 December; 33(12): 1293-1298 (Klienstiver II)). Kleinstiver et al. have also described modifications that improve the targeting fidelity of Cas9 (Nature, 2016 Jan. 28; 529, 490-495 (Kleinstiver III)). Modifications of PAM specificity relative to naturally occurring Cas9 reference molecules has been described by Kleinstiver et al. for both S. pyogenes (Kleinstiver et al., Nature. 2015 Jul. 23; 523(7561):481-5 (Kleinstiver I). Each of these references is incorporated by reference herein.

Modifications of PAM specificity relative to naturally occurring Cpf1 reference molecules has been described by Gao et al. (Gao et al., Nat Biotechnol. 2017 August; 35(8):789-792, which is incorporated by reference herein.) In certain embodiments, an RNA-guided nuclease can be an Cpf1 variant, e.g., an AsCpf1 variant. In certain embodiments, the Cpf1 variant is an AsCpf1 variant comprising an S542R/K607R variation, and which recognize TYCV PAM. In certain embodiments, the Cpf1 variant is an AsCpf1 variant comprising an S542R/K548V/N552R variation, and which recognize TATV PAM.

RNA-guided nucleases have been split into two or more parts, as described by Zetsche et al. (Nat Biotechnol. 2015 February; 33(2):139-42 (Zetsche II), incorporated by reference), and by Fine et al. (Sci Rep. 2015 Jul. 1; 5:10777 (Fine), incorporated by reference).

RNA-guided nucleases can be, in certain embodiments, size-optimized or truncated, for instance via one or more deletions that reduce the size of the nuclease while still retaining gRNA association, target and PAM recognition, and cleavage activities. In certain embodiments, RNA guided nucleases are bound, covalently or non-covalently, to another polypeptide, nucleotide, or other structure, optionally by means of a linker. Exemplary bound nucleases and linkers are described by Guilinger et al., Nature Biotechnology 32, 577-582 (2014), which is incorporated by reference for all purposes herein.

RNA-guided nucleases also optionally include a tag, such as, but not limited to, a nuclear localization signal to facilitate movement of RNA-guided nuclease protein into the nucleus. In certain embodiments, the RNA-guided nuclease can incorporate C- and/or N-terminal nuclear localization signals. Nuclear localization sequences are known in the art and are described in Maeder and elsewhere.

The foregoing list of modifications is intended to be exemplary in nature, and the skilled artisan will appreciate, in view of the instant disclosure, that other modifications may be possible or desirable in certain applications. For brevity, therefore, exemplary systems, methods and compositions of the present disclosure are presented with reference to particular RNA-guided nucleases, but it should be understood that the RNA-guided nucleases used may be modified in ways that do not alter their operating principles. Such modifications are within the scope of the present disclosure.

### Nucleic Acids Encoding RNA-Guided Nucleases

Nucleic acids encoding RNA-guided nucleases, e.g., Cpf1 or functional fragments thereof, are provided herein. Exemplary nucleic acids encoding RNA-guided nucleases have been described previously (see, e.g., Cong 2013; Wang 2013; Mali 2013; Jinek 2012).

In some cases, a nucleic acid encoding an RNA-guided nuclease can be a synthetic nucleic acid sequence. For example, the synthetic nucleic acid molecule can be chemically modified. In certain embodiments, an mRNA encoding an RNA-guided nuclease will have one or more (e.g., all) of the following properties: it can be capped; polyadenylated; and substituted with 5-methylcytidine and/or pseudouridine.

Synthetic nucleic acid sequences can also be codon optimized, e.g., at least one non-common codon or less-common codon has been replaced by a common codon. For example, the synthetic nucleic acid can direct the synthesis of an optimized messenger mRNA, e.g., optimized for expression in a mammalian expression system, e.g., described herein. Examples of codon optimized Cas9 coding sequences are presented in Cotta-Ramusino.

In addition, or alternatively, a nucleic acid encoding an RNA-guided nuclease may comprise a nuclear localization sequence (NLS). Nuclear localization sequences are known in the art.

### Functional Analysis of Candidate Molecules

Candidate RNA-guided nucleases, gRNAs, and complexes thereof, can be evaluated by standard methods known in the art. See, e.g. Cotta-Ramusino et al. The stability of RNP complexes may be evaluated by differential scanning fluorimetry, as described below.

Differential Scanning Fluorimetry (DSF)

The thermostability of ribonucleoprotein (RNP) complexes comprising gRNAs and RNA-guided nucleases can be measured via DSF. The DSF technique measures the thermostability of a protein, which can increase under favorable conditions such as the addition of a binding RNA molecule, e.g., a gRNA.

A DSF assay can be performed according to any suitable protocol, and can be employed in any suitable setting, including without limitation (a) testing different conditions (e.g., different stoichiometric ratios of gRNA: RNA-guided nuclease protein, different buffer solutions, etc.) to identify optimal conditions for RNP formation; and (b) testing modifications (e.g., chemical modifications, alterations of sequence, etc.) of an RNA-guided nuclease and/or a gRNA to identify those modifications that improve RNP formation or stability. One readout of a DSF assay is a shift in melting temperature of the RNP complex; a relatively high shift suggests that the RNP complex is more stable (and may thus have greater activity or more favorable kinetics of formation, kinetics of degradation, or another functional characteristic) relative to a reference RNP complex characterized by a lower shift. When the DSF assay is deployed as a screening tool, a threshold melting temperature shift may be specified, so that the output is one or more RNPs having a melting temperature shift at or above the threshold. For instance, the threshold can be 5-10° C. (e.g., 5°, 6°, 7°, 8°, 9°, 10°) or more, and the output may be one or more RNPs characterized by a melting temperature shift greater than or equal to the threshold.

Two non-limiting examples of DSF assay conditions are set forth below (while the conditions reference the use of Cas9, similar conditions can be employed with respect to Cpf1):

To determine the best solution to form RNP complexes, a fixed concentration (e.g., 2 μM) of Cas9 in water+10×SYPRO Orange® (Life Technologies cat # S-6650) is dispensed into a 384 well plate. An equimolar amount of gRNA diluted in solutions with varied pH and salt is then added. After incubating at room temperature for 10′ and brief centrifugation to remove any bubbles, a Bio-Rad CFX384™ Real-Time System C1000 Touch™ Thermal Cycler with the Bio-Rad CFX Manager software is used to run a gradient from 20° C. to 90° C. with a 1° C. increase in temperature every 10 seconds.

The second assay consists of mixing various concentrations of gRNA with fixed concentration (e.g. 2 μM) Cas9 in optimal buffer from assay 1 above and incubating (e.g. at RT for 10′) in a 384 well plate. An equal volume of optimal buffer+10×SYPRO Orange® (Life Technologies cat # S-6650) is added and the plate sealed with Microseal® B adhesive (MSB-1001). Following brief centrifugation to remove any bubbles, a Bio-Rad CFX384™ Real-Time System C1000 Touch™ Thermal Cycler with the Bio-Rad CFX Manager software is used to run a gradient from 20° C. to 90° C. with a 1° C. increase in temperature every 10 seconds.

### Genome Editing Strategies

The genome editing systems described above are used, in various embodiments of the present disclosure, to generate edits in (i.e., to modify) targeted regions of DNA within or obtained from a cell. Various strategies are described herein to generate particular edits, and these strategies are generally described in terms of the desired repair outcome, the number and positioning of individual edits (e.g., SSBs or DSBs), and the target sites of such edits.

Genome editing strategies that involve the formation of SSBs or DSBs are characterized by repair outcomes including: (a) deletion of all or part of a targeted region; (b) insertion into or replacement of all or part of a targeted region; or (c) interruption of all or part of a targeted region. This grouping is not intended to be limiting, or to be binding to any particular theory or model, and is offered solely for economy of presentation. Skilled artisans will appreciate that the listed outcomes are not mutually exclusive and that some repairs may result in other outcomes. The description of a particular editing strategy or method should not be understood to require a particular repair outcome unless otherwise specified.

Replacement of a targeted region generally involves the replacement of all or part of the existing sequence within the targeted region with a homologous sequence, for instance through gene correction or gene conversion, two repair outcomes that are mediated by HDR pathways. HDR is promoted by the use of a donor template, which can be single-stranded or double stranded, as described in greater detail below. Single or double stranded templates can be exogenous, in which case they will promote gene correction, or they can be endogenous (e.g., a homologous sequence within the cellular genome), to promote gene conversion. Exogenous templates can have asymmetric overhangs (i.e., the portion of the template that is complementary to the site of the DSB may be offset in a 3′ or 5′ direction, rather than being centered within the donor template), for instance as described by Richardson et al. (Nature Biotechnology 34, 339-344 (2016), (Richardson), incorporated by reference). In instances where the template is single stranded, it can correspond to either the complementary (top) or non-complementary (bottom) strand of the targeted region.

Gene conversion and gene correction are facilitated, in some cases, by the formation of one or more nicks in or around the targeted region, as described in Ran and Cotta-Ramusino et al. In some cases, a dual-nickase strategy is used to form two offset SSBs that, in turn, form a single DSB having an overhang (e.g., a 5′ overhang).

Interruption and/or deletion of all or part of a targeted sequence can be achieved by a variety of repair outcomes. As one example, a sequence can be deleted by simultaneously generating two or more DSBs that flank a targeted region, which is then excised when the DSBs are repaired, as is described in Maeder for the LCA10 mutation. As another example, a sequence can be interrupted by a deletion generated by formation of a double strand break with single-stranded overhangs, followed by exonucleolytic processing of the overhangs prior to repair.

One specific subset of target sequence interruptions is mediated by the formation of an indel within the targeted sequence, where the repair outcome is typically mediated by NHEJ pathways (including Alt-NHEJ). NHEJ is referred to as an “error prone” repair pathway because of its association with indel mutations. In some cases, however, a DSB is repaired by NHEJ without alteration of the sequence around it (a so-called “perfect” or “scarless” repair); this generally requires the two ends of the DSB to be perfectly ligated. Indels, meanwhile, are thought to arise from enzymatic processing of free DNA ends before they are ligated that adds and/or removes nucleotides from either or both strands of either or both free ends.

Because the enzymatic processing of free DSB ends may be stochastic in nature, indel mutations tend to be variable, occurring along a distribution, and can be influenced by a variety of factors, including the specific target site, the cell type used, the genome editing strategy used, etc. Even so, it is possible to draw limited generalizations about indel formation: deletions formed by repair of a single DSB are most commonly in the 1-50 bp range, but can reach greater than 100-200 bp. Insertions formed by repair of a single DSB tend to be shorter and often include short duplications of the sequence immediately surrounding the break site. However, it is possible to obtain large insertions, and in these cases, the inserted sequence has often been traced to other regions of the genome or to plasmid DNA present in the cells.

Indel mutations—and genome editing systems configured to produce indels—are useful for interrupting target sequences, for example, when the generation of a specific final sequence is not required and/or where a frameshift mutation would be tolerated. They can also be useful in settings where particular sequences are preferred, insofar as the certain sequences desired tend to occur preferentially from the repair of an SSB or DSB at a given site. Indel mutations are also a useful tool for evaluating or screening the activity of particular genome editing systems and their components. In these and other settings, indels can be characterized by (a) their relative and absolute frequencies in the genomes of cells contacted with genome editing systems and (b) the distribution of numerical differences relative to the unedited sequence, e.g., ±1, ±2, ±3, etc. As one example, in a lead-finding setting, multiple gRNAs can be screened to identify those gRNAs that most efficiently drive cutting at a target site based on an indel readout under controlled conditions. Guides that produce indels at or above a threshold frequency, or that produce a particular distribution of indels, can be selected for further study and development. Indel frequency and distribution can also be useful as a readout for evaluating different genome editing system implementations or formulations and delivery methods, for instance by keeping the gRNA constant and varying certain other reaction conditions or delivery methods.

Multiplex Strategies

While exemplary strategies discussed above have focused on repair outcomes mediated by single DSBs, genome editing systems according to this disclosure may also be employed to generate two or more DSBs, either in the same locus or in different loci. Strategies for editing that involve the formation of multiple DSBs, or SSBs, are described in, for instance, Cotta-Ramusino et al. As described herein, methods and compositions encompassed by the present disclosure can be used to affect T cell proliferation, survival, persistence, and/or function by modifying two or more T cell expressed gene(s), e.g., two or more of, three or more of, four or more of, five or more of, six or more of, seven or more of, eight or more, nine or more of or ten or more of the FAS, BID, CTLA4, PDCD1, CBLB, PTPN6, B2M, TRAC, CIITA and TRBC genes.

Donor Template Design

Donor template design is described in detail in the literature, for instance in Cotta-Ramusino. DNA oligomer donor templates (oligodeoxynucleotides or ODNs), which can be single stranded (ssODNs) or double-stranded (dsODNs), can be used to facilitate HDR-based repair of DSBs, and are particularly useful for introducing modifications into a target DNA sequence, inserting a new sequence into the target sequence, or replacing the target sequence altogether.

Whether single-stranded or double stranded, donor templates generally include regions that are homologous to regions of DNA within or near (e.g., flanking or adjoining) a target sequence to be cleaved. These homologous regions are referred to here as “homology arms,” and are illustrated schematically below:

[5′ homology arm]-[replacement sequence]--[3′ homology arm].

The homology arms can have any suitable length (including 0 nucleotides if only one homology arm is used), and 3′ and 5′ homology arms can have the same length, or can differ in length. The selection of appropriate homology arm lengths can be influenced by a variety of factors, such as the desire to avoid homologies or microhomologies with certain sequences such as Alu repeats or other very common elements. For example, a 5′ homology arm can be shortened to avoid a sequence repeat element. In other embodiments, a 3′ homology arm can be shortened to avoid a sequence repeat element. In certain embodiments, both the 5′ and the 3′ homology arms can be shortened to avoid including certain sequence repeat elements. In addition, some homology arm designs can improve the efficiency of editing or increase the frequency of a desired repair outcome. For example, Richardson et al. Nature Biotechnology 34, 339-344 (2016) (Richardson), which is incorporated by reference, found that the relative asymmetry of 3′ and 5′ homology arms of single stranded donor templates influenced repair rates and/or outcomes.

Replacement sequences in donor templates have been described elsewhere, including in Cotta-Ramusino et al. A replacement sequence can be any suitable length (including zero nucleotides, where the desired repair outcome is a deletion), and typically includes one, two, three or more sequence modifications relative to the naturally-occurring sequence within a cell in which editing is desired. One common sequence modification involves the alteration of the naturally-occurring sequence to repair a mutation that is related to a disease or condition of which treatment is desired. Another common sequence modification involves the alteration of one or more sequences that are complementary to, or code for, the PAM sequence of the RNA-guided nuclease or the targeting domain of the gRNA(s) being used to generate an SSB or DSB, to reduce or eliminate repeated cleavage of the target site after the replacement sequence has been incorporated into the target site.

Where a linear ssODN is used, it can be configured to (i) anneal to the nicked strand of the target nucleic acid, (ii) anneal to the intact strand of the target nucleic acid, (iii) anneal to the plus strand of the target nucleic acid, and/or (iv) anneal to the minus strand of the target nucleic acid. An ssODN may have any suitable length, e.g., about, at least, or no more than 150-200 nucleotides (e.g., 150, 160, 170, 180, 190, or 200 nucleotides).

It should be noted that a template nucleic acid can also be a nucleic acid vector, such as a viral genome or circular double stranded DNA, e.g., a plasmid. Nucleic acid vectors comprising donor templates can include other coding or non-coding elements. For example, a template nucleic acid can be delivered as part of a viral genome (e.g., in an AAV or lentiviral genome) that includes certain genomic backbone elements (e.g., inverted terminal repeats, in the case of an AAV genome) and optionally includes additional sequences coding for a gRNA and/or an RNA-guided nuclease. In certain embodiments, the donor template can be adjacent to, or flanked by, target sites recognized by one or more gRNAs, to facilitate the formation of free DSBs on one or both ends of the donor template that can participate in repair of corresponding SSBs or DSBs formed in cellular DNA using the same gRNAs. Exemplary nucleic acid vectors suitable for use as donor templates are described in Cotta-Ramusino et al.

Whatever format is used, a template nucleic acid can be designed to avoid undesirable sequences. In certain embodiments, one or both homology arms can be shortened to avoid overlap with certain sequence repeat elements, e.g., Alu repeats, LINE elements, etc.

### Targeted Integration

The present disclosure further provides genome editing systems comprising a donor template specifically designed to allow for the quantitative assessment of gene editing events that occur upon resolution of a cleavage event at a cleavage site of a target nucleic acid in a cell. The donor template of the genome editing systems described herein is a DNA oligodeoxynucleotides (ODNs), which can be single-stranded (ssODNs) or double-stranded (dsODNs), and can be used to facilitate HDR-based repair of a double-stranded break. The donor template is particularly useful for introducing modifications into a target DNA sequence, inserting a new sequence into the target sequence, or replacing the target sequence altogether. The disclosure provides donor templates comprising a cargo, one or two homology arms and one or more priming sites. The priming site(s) of the donor templates are spatially arranged in such a manner such that the frequency of integration of a portion of the donor template into the target nucleic acid may be readily assessed and quantified.

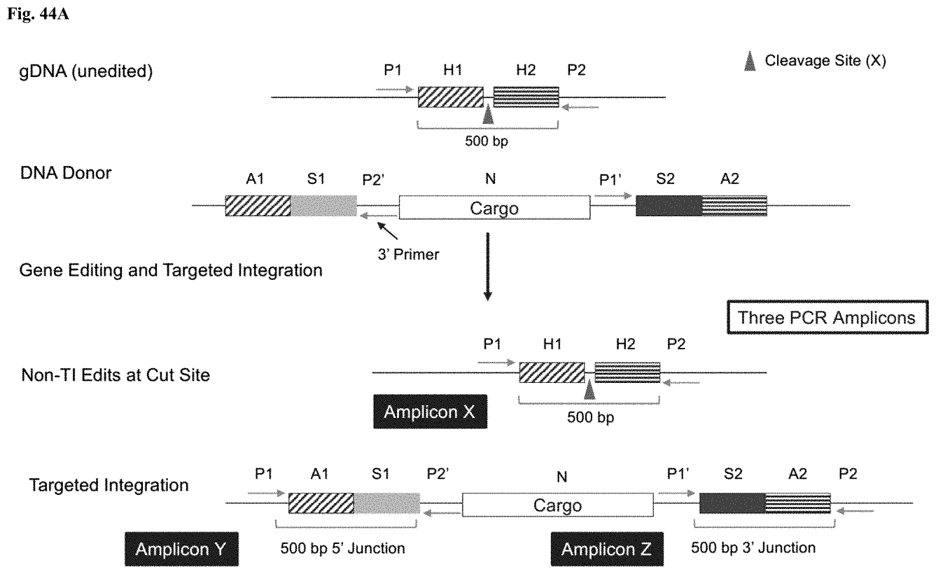

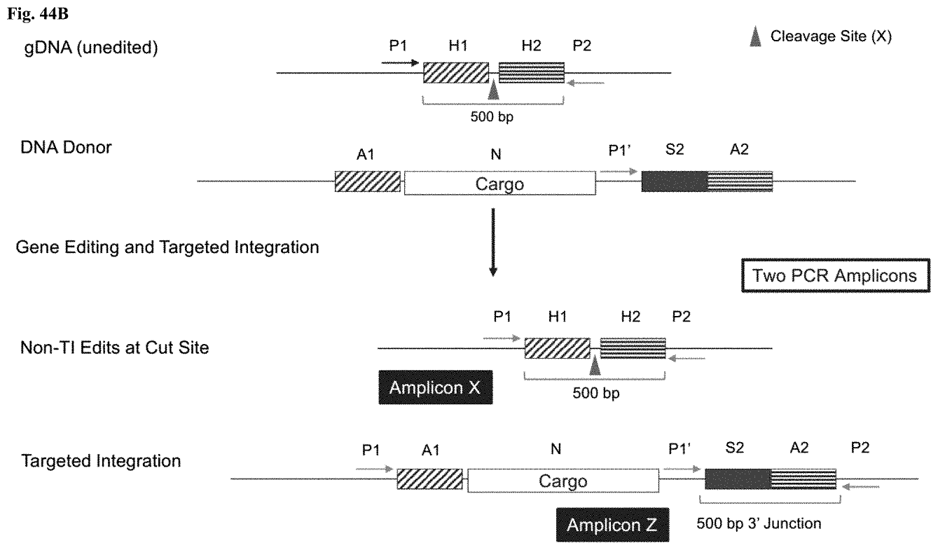

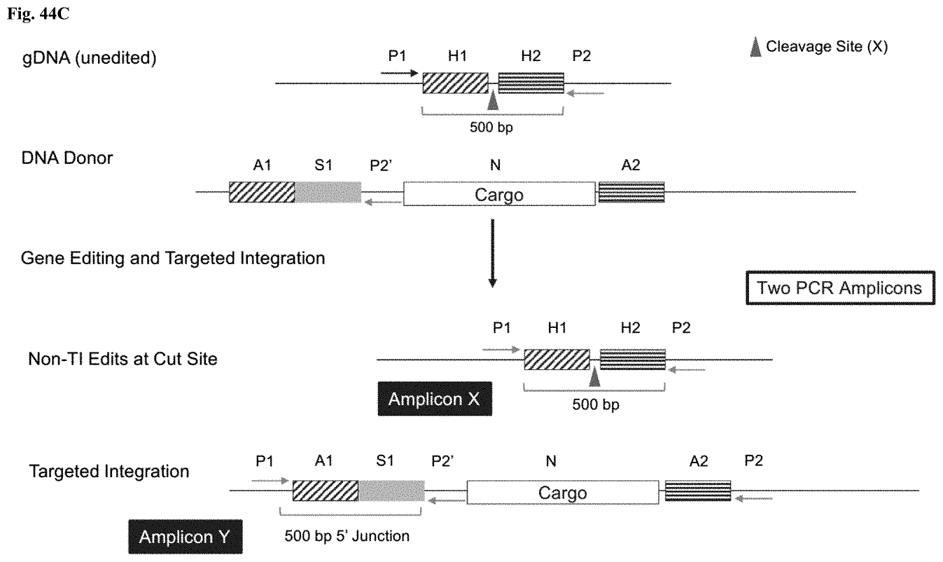

FIGS. 44A, 44B and 44C are diagrams illustrating representative donor templates and the potential targeted integration outcomes resulting from the use of these donor templates. The use of the exemplary donor templates described herein results in the targeted integration of at least one priming site in the targeted nucleic acid which may be used to generate an amplicon that can be sequenced to determine the frequency of targeted integration of a cargo (e.g., a transgene) to the targeted nucleic acid in the target cell.

For example, FIG. 44A illustrates an exemplary donor template comprising from 5′ to 3′, a first homology arm (A1), a first stuffer sequence (S1), a second priming site (P2′), a cargo, a first priming site, a second stuffer sequence, and a second homology arm. The first homology arm (A1) of the donor template is substantially identical to the first homology arm of the target nucleic acid, while the second homology arm (A2) of the donor template is substantially identical to the second homology arm of the target nucleic acid. The donor template is designed such that the second priming site (P2′) is substantially identical to the first priming site of the target nucleic acid (P1), and such that the first priming site (P1′) is substantially identical to the second priming site of the target nucleic acid (P2). Upon resolution of a target nucleic acid cleavage event using a nuclease described herein, a single primer pair set can be used to amplify the nucleic acid sequence surrounding the cleavage site of the target nucleic acid (i.e., the nucleic acid present between P1 and P2, between P1 and P2′, and between P1′ and P2). Advantageously, the size of the amplicons (illustrated as Amplicon X, Y and Z) resulting from resolution of a cleavage event without targeted integration or with targeted integration is approximately the same. The amplicons may then be assessed—for instance by sequencing, or hybridization to a probe sequence—to determine the frequency of targeted integration.

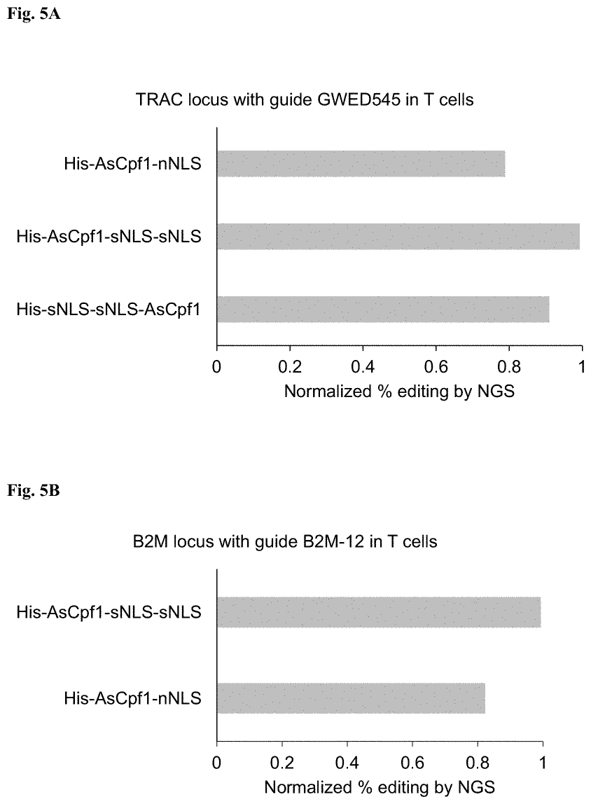

Alternatively, FIGS. 44B and 44C illustrate exemplary donor templates comprising a single priming site that is located either 3′ (FIG. 44B) or 5′ (FIG. 44C) from the cargo nucleic acid sequence. Again, upon resolution of a target nucleic acid cleavage event using a nuclease described herein, these exemplary donor templates are designed such that a single primer pair set can be used to amplify he nucleic acid sequence surrounding the cleavage site of the target nucleic acid, such that two amplicons of approximately the same size are obtained. When the priming site of the donor template is located 3′ from the cargo nucleic acid, amplicons corresponding to a non-targeted integration event, or an amplicon corresponding to the 5′ junction of the targeted integration site may be amplified. When the priming site of the donor template is located 5′ from the cargo nucleic acid, amplicons corresponding to a non-targeted integration event, or an amplicon corresponding to the 3′ junction of the targeted integration site may be amplified. These amplicons may be sequenced to determine the frequency of targeted integration.

Donor templates according to this disclosure may be implemented in any suitable way, including without limitation single stranded or double stranded DNA, linear or circular, naked or comprised within a vector, and/or associated, covalently or non-covalently (e.g., by direct hybridization or splint hybridization) with a guide RNA. In certain embodiments, the donor template is a ssODN. Where a linear ssODN is used, it can be configured to (i) anneal to a nicked strand of the target nucleic acid, (ii) anneal to the intact strand of the target nucleic acid, (iii) anneal to the plus strand of the target nucleic acid, and/or (iv) anneal to the minus strand of the target nucleic acid. An ssODN may have any suitable length, e.g., about, or no more than 150-200 nucleotides (e.g., 150, 160, 170, 180, 190, or 200 nucleotides). In other embodiments, the donor template is a dsODN. In certain embodiments, the donor template comprises a first strand. In another embodiment, a donor template comprises a first strand and a second strand. In certain embodiments, a donor template is an exogenous oligonucleotide, e.g., an oligonucleotide that is not naturally present in a cell.

It should be noted that a donor template can also be comprised within a nucleic acid vector, such as a viral genome or circular double-stranded DNA, e.g., a plasmid. In certain embodiments, the donor template can be a doggy-bone shaped DNA (see, e.g., U.S. Pat. No. 9,499,847). Nucleic acid vectors comprising donor templates can include other coding or non-coding elements. For example, a donor template nucleic acid can be delivered as part of a viral genome (e.g., in an AAV or lentiviral genome) that includes certain genomic backbone elements (e.g., inverted terminal repeats, in the case of an AAV genome) and optionally includes additional sequences coding for a gRNA and/or an RNA-guided nuclease. In certain embodiments, the donor template can be adjacent to, or flanked by, target sites recognized by one or more gRNAs, to facilitate the formation of free DSBs on one or both ends of the donor template that can participate in repair of corresponding SSBs or DSBs formed in cellular DNA using the same gRNAs. Exemplary nucleic acid vectors suitable for use as donor templates are described in Cotta-Ramusino et al.

Homology Arms

Whether single-stranded or double-stranded, donor templates generally include one or more regions that are homologous to regions of DNA, e.g., a target nucleic acid, within or near (e.g., flanking or adjoining) a target sequence to be cleaved, e.g., the cleavage site. These homologous regions are referred to here as “homology arms,” and are illustrated schematically below:

[5′ homology arm]-[replacement sequence]--[3′ homology arm].

The homology arms of the donor templates described herein may be of any suitable length, provided such length is sufficient to allow efficient resolution of a cleavage site on a targeted nucleic acid by a DNA repair process requiring a donor template. In certain embodiments, where amplification by, e.g., PCR, of the homology arm is desired, the homology arm is of a length such that the amplification may be performed. In certain embodiments, where sequencing of the homology arm is desired, the homology arm is of a length such that the sequencing may be performed. In certain embodiments, where quantitative assessment of amplicons is desired, the homology arms are of such a length such that a similar number of amplifications of each amplicon is achieved, e.g., by having similar G/C content, amplification temperatures, etc. In certain embodiments, the homology arm is double-stranded. In certain embodiments, the double stranded homology arm is single stranded.

In certain embodiments, the 5′ homology arm is between 50 to 250 nucleotides in length. In certain embodiments, the 5′ homology arm is between 50-2000 nucleotides in length. In certain embodiments, the 5′ homology arm is between 50-1500 nucleotides in length. In certain embodiments, the 5′ homology arm is between 50-1000 nucleotides in length. In certain embodiments, the 5′ homology arm is between 50-500 nucleotides in length. In certain embodiments, the 5′ homology arm is between 150 to 250 nucleotides in length. In certain embodiments, the 5′ homology arm is 2000 nucleotides or less in length. In certain embodiments, the 5′ homology arm is 1500 nucleotides or less in length. In certain embodiments, the 5′ homology arm is 1000 nucleotides or less in length. In certain embodiments, the 5′ homology arm is 700 nucleotides or less in length. In certain embodiments, the 5′ homology arm is 650 nucleotides or less in length. In certain embodiments, the 5′ homology arm is 600 nucleotides or less in length. In certain embodiments, the 5′ homology arm is 550 nucleotides or less in length. In certain embodiments, the 5′ homology arm is 500 nucleotides or less in length. In certain embodiments, the 5′ homology arm is 400 nucleotides or less in length. In certain embodiments, the 5′ homology arm is 300 nucleotides or less in length. In certain embodiments, the 5′ homology arm is 250 nucleotides or less in length. In certain embodiments, the 5′ homology arm is 200 nucleotides or less in length. In certain embodiments, the 5′ homology arm is 150 nucleotides or less in length. In certain embodiments, the 5′ homology arm is less than 100 nucleotides in length. In certain embodiments, the 5′ homology arm is 50 nucleotides in length or less. In certain embodiments, the 5′ homology arm is 250, 200, 190, 180, 170, 160, 150, 140, 130, 120, 110, 100, 90, 80, 70, 60, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, or 20 nucleotides in length. In certain embodiments, the 5′ homology arm is at least 20 nucleotides in length. In certain embodiments, the 5′ homology arm is at least 40 nucleotides in length. In certain embodiments, the 5′ homology arm is at least 50 nucleotides in length. In certain embodiments, the 5′ homology arm is at least 70 nucleotides in length. In certain embodiments, the 5′ homology arm is at least 100 nucleotides in length. In certain embodiments, the 5′ homology arm is at least 200 nucleotides in length. In certain embodiments, the 5′ homology arm is at least 300 nucleotides in length. In certain embodiments, the 5′ homology arm is at least 400 nucleotides in length. In certain embodiments, the 5′ homology arm is at least 500 nucleotides in length. In certain embodiments, the 5′ homology arm is at least 600 nucleotides in length. In certain embodiments, the 5′ homology arm is at least 700 nucleotides in length. In certain embodiments, the 5′ homology arm is at least 1000 nucleotides in length. In certain embodiments, the 5′ homology arm is at least 1500 nucleotides in length. In certain embodiments, the 5′ homology arm is at least 2000 nucleotides in length. In certain embodiments, the 5′ homology arm is about 20 nucleotides in length. In certain embodiments, the 5′ homology arm is about 40 nucleotides in length. In certain embodiments, the 5′ homology arm is 250 nucleotides in length or less. In certain embodiments, the 5′ homology arm is about 100 nucleotides in length. In certain embodiments, the 5′ homology arm is about 200 nucleotides in length.

In certain embodiments, the 3′ homology arm is between 50 to 250 nucleotides in length. In certain embodiments, the 3′ homology arm is between 50-2000 nucleotides in length. In certain embodiments, the 3′ homology arm is between 50-1500 nucleotides in length. In certain embodiments, the 3′ homology arm is between 50-1000 nucleotides in length. In certain embodiments, the 3′ homology arm is between 50-500 nucleotides in length. In certain embodiments, the 3′ homology arm is between 150 to 250 nucleotides in length. In certain embodiments, the 3′ homology arm is 2000 nucleotides or less in length. In certain embodiments, the 3′ homology arm is 1500 nucleotides or less in length. In certain embodiments, the 3′ homology arm is 1000 nucleotides or less in length. In certain embodiments, the 3′ homology arm is 700 nucleotides or less in length. In certain embodiments, the 3′ homology arm is 650 nucleotides or less in length. In certain embodiments, the 3′ homology arm is 600 nucleotides or less in length. In certain embodiments, the 3′ homology arm is 550 nucleotides or less in length. In certain embodiments, the 3′ homology arm is 500 nucleotides or less in length. In certain embodiments, the 3′ homology arm is 400 nucleotides or less in length. In certain embodiments, the 3′ homology arm is 300 nucleotides or less in length. In certain embodiments, the 3′ homology arm is 200 nucleotides in length or less. In certain embodiments, the 3′ homology arm is 150 nucleotides in length or less. In certain embodiments, the 3′ homology arm is 100 nucleotides in length or less. In certain embodiments, the 3′ homology arm is 50 nucleotides in length or less. In certain embodiments, the 3′ homology arm is 250, 200, 190, 180, 170, 160, 150, 140, 130, 120, 110, 100, 90, 80, 70, 60, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, or 20 nucleotides in length. In certain embodiments, the 3′ homology arm is at least 20 nucleotides in length. In certain embodiments, the 3′ homology arm is at least 40 nucleotides in length. In certain embodiments, the 3′ homology arm is at least 50 nucleotides in length. In certain embodiments, the 3′ homology arm is at least 70 nucleotides in length. In certain embodiments, the 3′ homology arm is at least 100 nucleotides in length. In certain embodiments, the 3′ homology arm is at least 200 nucleotides in length. In certain embodiments, the 3′ homology arm is at least 300 nucleotides in length. In certain embodiments, the 3′ homology arm is at least 400 nucleotides in length. In certain embodiments, the 3′ homology arm is at least 500 nucleotides in length. In certain embodiments, the 3′ homology arm is at least 600 nucleotides in length. In certain embodiments, the 3′ homology arm is at least 700 nucleotides in length. In certain embodiments, the 3′ homology arm is at least 1000 nucleotides in length. In certain embodiments, the 3′ homology arm is at least 1500 nucleotides in length. In certain embodiments, the 3′ homology arm is at least 2000 nucleotides in length. In certain embodiments, the 3′ homology arm is about 20 nucleotides in length. In certain embodiments, the 3′ homology arm is about 40 nucleotides in length. In certain embodiments, the 3′ homology arm is 250 nucleotides in length or less. In certain embodiments, the 3′ homology arm is about 100 nucleotides in length. In certain embodiments, the 3′ homology arm is about 200 nucleotides in length.

In certain embodiments, the 5′ homology arm is between 50 to 250 base pairs in length. In certain embodiments, the 5′ homology arm is between 50-2000 base pairs in length. In certain embodiments, the 5′ homology arm is between 50-1500 base pairs in length. In certain embodiments, the 5′ homology arm is between 50-1000 base pairs in length. In certain embodiments, the 5′ homology arm is between 50-500 base pairs in length. In certain embodiments, the 5′ homology arm is between 150 base pairs to 250 base pairs in length. In certain embodiments, the 5′ homology arm is 2000 base pairs or less in length. In certain embodiments, the 5′ homology arm is 1500 base pairs or less in length. In certain embodiments, the 5′ homology arm is 1000 base pairs or less in length. In certain embodiments, the 5′ homology arm is 700 base pairs or less in length.

In certain embodiments, the 5′ homology arm is 650 base pairs or less in length. In certain embodiments, the 5′ homology arm is 600 base pairs or less in length. In certain embodiments, the 5′ homology arm is 550 base pairs or less in length. In certain embodiments, the 5′ homology arm is 500 base pairs or less in length. In certain embodiments, the 5′ homology arm is 400 base pairs or less in length. In certain embodiments, the 5′ homology arm is 300 base pairs or less in length. In certain embodiments, the 5′ homology arm is 250 base pairs or less in length. In certain embodiments, the 5′ homology arm is 200 base pairs or less in length. In certain embodiments, the 5′ homology arm is 150 base pairs or less in length. In certain embodiments, the 5′ homology arm is less than 100 base pairs in length. In certain embodiments, the 5′ homology arm is 50 base pairs in length or less. In certain embodiments, the 5′ homology arm is 250, 200, 190, 180, 170, 160, 150, 140, 130, 120, 110, 100, 90, 80, 70, 60, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, or 20 base pairs in length. In certain embodiments, the 5′ homology arm is at least 20 base pairs in length. In certain embodiments, the 5′ homology arm is at least 40 base pairs in length. In certain embodiments, the 5′ homology arm is at least 50 base pairs in length. In certain embodiments, the 5′ homology arm is at least 70 base pairs in length. In certain embodiments, the 5′ homology arm is at least 100 base pairs in length. In certain embodiments, the 5′ homology arm is at least 200 base pairs in length. In certain embodiments, the 5′ homology arm is at least 300 base pairs in length. In certain embodiments, the 5′ homology arm is at least 400 base pairs in length. In certain embodiments, the 5′ homology arm is at least 500 base pairs in length. In certain embodiments, the 5′ homology arm is at least 600 base pairs in length. In certain embodiments, the 5′ homology arm is at least 700 base pairs in length. In certain embodiments, the 5′ homology arm is at least 1000 base pairs in length. In certain embodiments, the 5′ homology arm is at least 1500 base pairs in length. In certain embodiments, the 5′ homology arm is at least 2000 base pairs in length. In certain embodiments, the 5′ homology arm is about 20 base pairs in length. In certain embodiments, the 5′ homology arm is about 40 base pairs in length. In certain embodiments, the 5′ homology arm is 250 base pairs in length or less. In certain embodiments, the 5′ homology arm is about 100 base pairs in length. In certain embodiments, the 5′ homology arm is about 200 base pairs in length.

In certain embodiments, the 3′ homology arm is between 50 to 250 base pairs in length. In certain embodiments, the 3′ homology arm is between 50-2000 base pairs in length. In certain embodiments, the 3′ homology arm is between 50-1500 base pairs in length. In certain embodiments, the 3′ homology arm is between 50-1000 base pairs in length. In certain embodiments, the 3′ homology arm is between 50-500 base pairs in length. In certain embodiments, the 3′ homology arm is between 150 base pairs to 250 base pairs in length. In certain embodiments, the 3′ homology arm is 2000 base pairs or less in length. In certain embodiments, the 3′ homology arm is 1500 base pairs or less in length. In certain embodiments, the 3′ homology arm is 1000 base pairs or less in length. In certain embodiments, the 3′ homology arm is 700 base pairs or less in length. In certain embodiments, the 3′ homology arm is 650 base pairs or less in length. In certain embodiments, the 3′ homology arm is 600 base pairs or less in length. In certain embodiments, the 3′ homology arm is 550 base pairs or less in length. In certain embodiments, the 3′ homology arm is 500 base pairs or less in length. In certain embodiments, the 3′ homology arm is 400 base pairs or less in length. In certain embodiments, the 3′ homology arm is 300 base pairs or less in length. In certain embodiments, the 3′ homology arm is 250 base pairs or less in length. In certain embodiments, the 3′ homology arm is 200 base pairs or less in length. In certain embodiments, the 3′ homology arm is 150 base pairs or less in length. In certain embodiments, the 3′ homology arm is less than 100 base pairs in length. In certain embodiments, the 3′ homology arm is 50 base pairs in length or less. In certain embodiments, the 3′ homology arm is 250, 200, 190, 180, 170, 160, 150, 140, 130, 120, 110, 100, 90, 80, 70, 60, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, or 20 base pairs in length. In certain embodiments, the 3′ homology arm is at least 20 base pairs in length. In certain embodiments, the 3′ homology arm is at least 40 base pairs in length. In certain embodiments, the 3′ homology arm is at least 50 base pairs in length. In certain embodiments, the 3′ homology arm is at least 70 base pairs in length. In certain embodiments, the 3′ homology arm is at least 100 base pairs in length. In certain embodiments, the 3′ homology arm is at least 200 base pairs in length. In certain embodiments, the 3′ homology arm is at least 300 base pairs in length. In certain embodiments, the 3′ homology arm is at least 400 base pairs in length. In certain embodiments, the 3′ homology arm is at least 500 base pairs in length. In certain embodiments, the 3′ homology arm is at least 600 base pairs in length. In certain embodiments, the 3′ homology arm is at least 700 base pairs in length. In certain embodiments, the 3′ homology arm is at least 1000 base pairs in length. In certain embodiments, the 3′ homology arm is at least 1500 base pairs in length. In certain embodiments, the 3′ homology arm is at least 2000 base pairs in length. In certain embodiments, the 3′ homology arm is about 20 base pairs in length. In certain embodiments, the 3′ homology arm is about 40 base pairs in length. In certain embodiments, the 3′ homology arm is 250 base pairs in length or less. In certain embodiments, the 3′ homology arm is about 100 base pairs in length. In certain embodiments, the 3′ homology arm is about 200 base pairs in length. In certain embodiments, the 3′ homology arm is 250 base pairs in length or less. In certain embodiments, the 3′ homology arm is 200 base pairs in length or less. In certain embodiments, the 3′ homology arm is 150 base pairs in length or less. In certain embodiments, the 3′ homology arm is 100 base pairs in length or less. In certain embodiments, the 3′ homology arm is 50 base pairs in length or less. In certain embodiments, the 3′ homology arm is 250, 200, 190, 180, 170, 160, 150, 140, 130, 120, 110, 100, 90, 80, 70, 60, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, or 20 base pairs in length. In certain embodiments, the 3′ homology arm is 40 base pairs in length.

The 5′ and 3′ homology arms can be of the same length or can differ in length. In certain embodiments, the 5′ and 3′ homology arms are amplified to allow for the quantitative assessment of gene editing events, such as targeted integration, at a target nucleic acid. In certain embodiments, the quantitative assessment of the gene editing events may rely on the amplification of both the 5′ junction and 3′ junction at the site of targeted integration by amplifying the whole or a part of the homology arm using a single pair of PCR primers in a single amplification reaction. Accordingly, although the length of the 5′ and 3′ homology arms may differ, the length of each homology arm should be capable of amplification (e.g., using PCR), as desired. Moreover, when amplification of both the 5′ and the difference in lengths of the 5′ and 3′ homology arms in a single PCR reaction is desired, the length difference between the 5′ and 3′ homology arms should allow for PCR amplification using a single pair of PCR primers.

In certain embodiments, the length of the 5′ and 3′ homology arms do not differ by more than 75 nucleotides. Thus, in certain embodiments, when the 5′ and 3′ homology arms differ in length, the length difference between the homology arms is less than 70, 60, 50, 40, 30, 20, 10, 9, 8, 7, 6, 5, 4, 3, 2, 1 nucleotides or base pairs. In certain embodiments, the 5′ and 3′ homology arms differ in length by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75 nucleotides. In certain embodiments, the length difference between the 5′ and 3′ homology arms is less than 70, 60, 50, 40, 30, 20, 10, 9, 8, 7, 6, 5, 4, 3, 2, 1 base pairs. In certain embodiments, the 5′ and 3′ homology arms differ in length by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75 base pairs.

Donor templates of the disclosure are designed to facilitate homologous recombination with a target nucleic acid having a cleavage site, wherein the target nucleic acid comprises, from 5′ to 3′,

P1--H1--X--H2--P2,

wherein P1 is a first priming site; H1 is a first homology arm; X is the cleavage site; H2 is a second homology arm; and P2 is a second priming site; and wherein the donor template comprises, from 5′ to 3′,

A1--P2′--N--A2, or A1--N--P1′--A2,

wherein A1 is a homology arm that is substantially identical to H1; P2′ is a priming site that is substantially identical to P2; N is a cargo; P1′ is a priming site that is substantially identical to P1; and A2 is a homology arm that is substantially identical to H2. In certain embodiments, the target nucleic acid is double stranded. In certain embodiments, the target nucleic acid comprises a first strand and a second strand. In another embodiment, the target nucleic acid is single stranded. In certain embodiments, the target nucleic acid comprises a first strand.

In certain embodiments, the donor template comprises, from 5′ to 3′,

A1--P2′--N--A2.

In certain embodiments, the donor template comprises, from 5′ to 3′,

A1--P2′--N--P1′--A2.

In certain embodiments, the target nucleic acid comprises, from 5′ to 3′,

P1--H1--X--H2--P2,

wherein P1 is a first priming site; H1 is a first homology arm; X is the cleavage site; H2 is a second homology arm; and P2 is a second priming site; and the first strand of the donor template comprises, from 5′ to 3′,

A1--P2′--N--A2, or A1--N--P1′--A2,

wherein A1 is a homology arm that is substantially identical to H1; P2′ is a priming site that is substantially identical to P2; N is a cargo; P1′ is a priming site that is substantially identical to P1; and A2 is a homology arm that is substantially identical to H2.

In certain embodiments, a first strand of the donor template comprises, from 5′ to 3′,

A1--P2′--N--P1′--A2.

In certain embodiments, a first strand of the donor template comprises, from 5′ to 3′,

A1--N--P1′--A2.

In certain embodiments, A1 is 700 base pairs or less in length. In certain embodiments, A1 is 650 base pairs or less in length. In certain embodiments, A1 is 600 base pairs or less in length. In certain embodiments, A1 is 550 base pairs or less in length. In certain embodiments, A1 is 500 base pairs or less in length. In certain embodiments, A1 is 400 base pairs or less in length. In certain embodiments, A1 is 300 base pairs or less in length. In certain embodiments, A1 is less than 250 base pairs in length. In certain embodiments, A1 is less than 200 base pairs in length. In certain embodiments, A1 is less than 150 base pairs in length. In certain embodiments, A1 is less than 100 base pairs in length. In certain embodiments, A1 is less than 50 base pairs in length. In certain embodiments, the A1 is 250, 200, 190, 180, 170, 160, 150, 140, 130, 120, 110, 100, 90, 80, 70, 60, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, or 20 base pairs in length. In certain embodiments, A1 is 40 base pairs in length. In certain embodiments, A1 is 30 base pairs in length. In certain embodiments, A1 is 20 base pairs in length.

In certain embodiments, A2 is 700 base pairs or less in length. In certain embodiments, A2 is 650 base pairs or less in length. In certain embodiments, A2 is 600 base pairs or less in length. In certain embodiments, A2 is 550 base pairs or less in length. In certain embodiments, A2 is 500 base pairs or less in length. In certain embodiments, A2 is 400 base pairs or less in length. In certain embodiments, A2 is 300 base pairs or less in length. In certain embodiments, A2 is less than 250 base pairs in length. In certain embodiments, A2 is less than 200 base pairs in length. In certain embodiments, A2 is less than 150 base pairs in length. In certain embodiments, A2 is less than 100 base pairs in length. In certain embodiments, A2 is less than 50 base pairs in length. In certain embodiments, A2 is 250, 200, 190, 180, 170, 160, 150, 140, 130, 120, 110, 100, 90, 80, 70, 60, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, or 20 base pairs in length. In certain embodiments, A2 is 40 base pairs in length. In certain embodiments, A2 is 30 base pairs in length. In certain embodiments, A2 is 20 base pairs in length.

In certain embodiments, A1 is 700 nucleotides or less in length. In certain embodiments, A1 is 650 nucleotides or less in length. In certain embodiments, A1 is 600 nucleotides or less in length. In certain embodiments, A1 is 550 nucleotides or less in length. In certain embodiments, A1 is 500 nucleotides or less in length. In certain embodiments, A1 is 400 nucleotides or less in length. In certain embodiments, A1 is 300 nucleotides or less in length. In certain embodiments, A1 is less than 250 nucleotides in length. In certain embodiments, A1 is less than 200 nucleotides in length. In certain embodiments, A1 is less than 150 nucleotides in length. In certain embodiments, A1 is less than 100 nucleotides in length. In certain embodiments, A1 is less than 50 nucleotides in length. In certain embodiments, the A1 is 250, 200, 190, 180, 170, 160, 150, 140, 130, 120, 110, 100, 90, 80, 70, 60, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, or 20 nucleotides in length. In certain embodiments, A1 is at least 40 nucleotides in length. In certain embodiments, A1 is at least 30 nucleotides in length. In certain embodiments, A1 is at least 20 nucleotides in length.

In certain embodiments, A2 is 700 nucleotides or less in length. In certain embodiments, A2 is 650 base pairs or less in length. In certain embodiments, A2 is 600 nucleotides or less in length. In certain embodiments, A2 is 550 nucleotides or less in length. In certain embodiments, A2 is 500 nucleotides or less in length. In certain embodiments, A2 is 400 nucleotides or less in length. In certain embodiments, A2 is 300 nucleotides or less in length. In certain embodiments, A2 is less than 250 nucleotides in length. In certain embodiments, A2 is less than 200 nucleotides in length. In certain embodiments, A2 is less than 150 nucleotides in length. In certain embodiments, A2 is less than 100 nucleotides in length. In certain embodiments, A2 is less than 50 nucleotides in length. In certain embodiments, A2 is 250, 200, 190, 180, 170, 160, 150, 140, 130, 120, 110, 100, 90, 80, 70, 60, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, or 20 nucleotides in length. In certain embodiments, A2 is at least 40 nucleotides in length. In certain embodiments, A2 is at least 30 nucleotides in length. In certain embodiments, A2 is at least 20 nucleotides in length.

In certain embodiments, the nucleic acid sequence of A1 is substantially identical to the nucleic acid sequence of H1. In certain embodiments A1 has a sequence that is identical to, or differs by no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides from H1. In certain embodiments A1 has a sequence that is identical to, or differs by no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 base pairs from H1.

In certain embodiments, the nucleic acid sequence of A2 is substantially identical to the nucleic acid sequence of H2. In certain embodiments A2 has a sequence that is identical to, or differs by no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides from H2. In certain embodiments A2 has a sequence that is identical to, or differs by no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 base pairs from H2.

Whatever format is used, a donor template can be designed to avoid undesirable sequences. In certain embodiments, one or both homology arms can be shortened to avoid overlap with certain sequence repeat elements, e.g., Alu repeats, LINE elements, etc.

Priming Sites

The donor templates described herein comprise at least one priming site having a sequence that is substantially similar to, or identical to, the sequence of a priming site within the target nucleic acid, but is in a different spatial order or orientation relative to a homology sequence/homology arm in the donor template. When the donor template is homologously recombined with the target nucleic acid, the priming site(s) are advantageously incorporated into the target nucleic acid, thereby allowing for the amplification of a portion of the modified nucleic acid sequence that results from the recombination event. In certain embodiments, the donor template comprises at least one priming site. In certain embodiments, the donor template comprises a first and a second priming site. In certain embodiments, the donor template comprises three or more priming sites.

In certain embodiments, the donor template comprises a priming site P1′, that is substantially similar or identical to a priming site, P1, within the target nucleic acid, wherein upon integration of the donor template at the target nucleic acid, P1′, is incorporated downstream from P1. In certain embodiments, the donor template comprises a first priming site, P1′, and a second priming site, P2′; wherein, P1′, is substantially similar or identical to a first priming site, P1, within the target nucleic acid; wherein P2′ is substantially similar or identical to second priming site, P2, within the target nucleic acid; and wherein P1 and P2 are not substantially similar or identical. In certain embodiments, the donor template comprises a first priming site, P1′, and a second priming site, P2′; wherein, P1′, is substantially similar or identical to a first priming site, P1, within the target nucleic acid; wherein P2′ is substantially similar or identical to second priming site, P2, within the target nucleic acid; wherein P2 is located downstream from P1 on the target nucleic acid; wherein P1 and P2 are not substantially similar or identical; and wherein upon integration of the donor template at the target nucleic acid, P1′, is incorporated downstream from P1. P2′ is incorporated upstream from P2, and P2′ is incorporated upstream from P1.

In certain embodiments, the target nucleic acid comprises a first priming site (P1) and a second priming site (P2). The first priming site in the target nucleic acid may be within the first homology arm. Alternatively, the first priming site in the target nucleic acid may be 5′ and adjacent to the first homology arm. The second priming site in the target nucleic acid may be within the second homology arm. Alternatively, the second priming site in the target nucleic acid may be 3′ and adjacent to the second homology arm.

The donor template may comprise a cargo sequence, a first priming site (P1′), and a second priming site (P2′), wherein P2′ is located 5′ from the cargo sequence, wherein P1′ is located 3′ from the cargo sequence (i.e., A1--P2′--N--P1′--A2), wherein P1′ is substantially identical to P1, and wherein P2′ is substantially identical to P2. In this scenario, a primer pair comprising an oligonucleotide targeting P1′ and P1 and an oligonucleotide comprising P2′ and P2 may be used to amplify the targeted locus, thereby generation three amplicons of similar size which may be sequenced to determine whether targeted integration has occurred. The first amplicon, Amplicon X, results from the amplification of the nucleic acid sequence between P1 and P2 as a result of non-targeted integration at the target nucleic acid. The second amplicon, Amplicon Y, results from the amplification of the nucleic acid sequence between P1 and P2′ following a targeted integration event at the target nucleic acid, thereby amplifying the 5′ junction. The third amplicon, Amplicon Z, results from the amplification of the nucleic acid sequence between P1′ and P2 following a targeted integration event at the target nucleic acid, thereby amplifying the 3′ junction. In other embodiments, P1′ may be identical to P1. Moreover, P2′ may be identical to P2.

In certain embodiments, the donor template comprises a cargo and a priming site (P1′), wherein P1′ is located 3′ from the cargo nucleic acid sequence (rnp A1--N--P1′--A2) and P1′ is substantially identical to P1. In this scenario, a primer pair comprising an oligonucleotide targeting P1′ and P1 and an oligonucleotide targeting P2 may be used to amplify the targeted locus, thereby generation two amplicons of similar size which may be sequenced to determine whether targeted integration has occurred. The first amplicon, Amplicon X, results from the amplification of the nucleic acid sequence between P1 and P2 as a result of non-targeted integration at the target nucleic acid. The second amplicon, Amplicon Z, results from the amplification of the nucleic acid sequence between P1′ and P2 following a targeted integration event at the target nucleic acid, thereby amplifying the 3′ junction. In other embodiments, P1′ may be identical to P1. Moreover, P2′ may be identical to P2.

In certain embodiments, the target nucleic acid comprises a first priming site (P1) and a second priming site (P2), and the donor template comprises a priming site P2′, wherein P2′ is located 5′ from the cargo nucleic acid sequence (i.e., A1--P2′--N--A2), and P2′ is substantially identical to P2. In this scenario, a primer pair comprising an oligonucleotide targeting P2′ and P2 and an oligonucleotide targeting P1 may be used to amplify the targeted locus, thereby generation two amplicons of similar size which may be sequenced to determine whether targeted integration has occurred. The first amplicon, Amplicon X, results from the amplification of the nucleic acid sequence between P1 and P2 as a result of non-targeted integration at the target nucleic acid. The second amplicon, Amplicon Y, results from the amplification of the nucleic acid sequence between P1 and P2′ following a targeted integration event at the target nucleic acid, thereby amplifying the 5′ junction. In other embodiments, P1′ may be identical to P1. Moreover, P2′ may be identical to P2.

A priming site of the donor template may be of any length that allows for the quantitative assessment of gene editing events at a target nucleic acid by amplification and/or sequencing of a portion of the target nucleic acid. For example, in certain embodiments, the target nucleic acid comprises a first priming site (P1) and the donor template comprises a priming site (P1′). In these embodiments, the length of the P1′ priming site and the P1 primer site is such that a single primer can specifically anneal to both priming sites (for example, in certain embodiments, the length of the P1′ priming site and the P1 priming site is such that both have the same or very similar GC content).

In certain embodiments, the priming site of the donor template is 60 nucleotides in length. In certain embodiments, the priming site of the donor template is less than 60 nucleotides in length. In certain embodiments, the priming site of the donor template is less than 50 nucleotides in length. In certain embodiments, the priming site of the donor template is less than 40 nucleotides in length. In certain embodiments, the priming site of the donor template is less than 30 nucleotides in length. In certain embodiments the priming site of the donor template is 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 nucleotides in length. In certain embodiments, the priming site of the donor template is 60 base pairs in length. In certain embodiments, the priming site of the donor template is less than 60 base pairs in length. In certain embodiments, the priming site of the donor template is less than 50 base pairs in length. In certain embodiments, the priming site of the donor template is less than 40 base pairs in length. In certain embodiments, the priming site of the donor template is less than 30 base pairs in length. In certain embodiments the priming site of the donor template is 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 base pairs in length.

In certain embodiments, upon resolution of the cleavage event at the cleavage site in the target nucleic acid and homologous recombination of the donor template with the target nucleic acid, the distance between the first priming site of the target nucleic acid (P1) and now integrated P2′ priming site is 600 base pairs or less. In certain embodiments, upon resolution of the cleavage event and homologous recombination of the donor template with the target nucleic acid, the distance between the first priming site of the target nucleic acid (P1) and now integrated P2′ priming site is 550, 500, 450, 400, 350, 300, 250, 200, 150 base pairs or less. In certain embodiments, upon resolution of the cleavage event at the target nucleic acid and homologous recombination of the donor template with the target nucleic acid, the distance between the first priming site of the target nucleic acid (P1) and now integrated P2′ priming site is 600 nucleotides or less. In certain embodiments, upon resolution of the cleavage event at the target nucleic acid and homologous recombination of the donor template with the target nucleic acid, the distance between the first priming site of the target nucleic acid (P1) and now integrated P2′ priming site is 550, 500, 450, 400, 350, 300, 250, 200, 150 nucleotides or less.

In certain embodiments, the target nucleic acid comprises a second priming site (P2) and the donor template comprises a priming site (P2′) that is substantially identical to P2. In certain embodiments, upon resolution of the cleavage event at the target nucleic acid and homologous recombination of the donor template with the target nucleic acid, the distance between the second priming site of the target nucleic acid (P2) and now integrated P1′ priming site is 600 base pairs or less. In certain embodiments, upon resolution of the cleavage event at the target nucleic acid and homologous recombination of the donor template with the target nucleic acid, the distance between the second priming site of the target nucleic acid (P2) and now integrated P1′ priming site is 550, 500, 450, 400, 350, 300, 250, 200, 150 base pairs or less. In certain embodiments, upon resolution of the cleavage event at the target nucleic acid and homologous recombination of the donor template with the target nucleic acid, the distance between the second priming site of the target nucleic acid (P2) and now integrated P1′ priming site is 600 nucleotides or less. In certain embodiments, upon resolution of the cleavage event at the target nucleic acid and homologous recombination of the donor template with the target nucleic acid, the distance between the second priming site of the target nucleic acid (P2) and now integrated P1′ priming site is 550, 500, 450, 400, 350, 300, 250, 200, 150 nucleotides or less.

In certain embodiments, the nucleic acid sequence of P2′ is comprised within the nucleic acid sequence of A1. In certain embodiments, the nucleic acid sequence of P2′ is immediately adjacent to the nucleic acid sequence of A1. In certain embodiments, the nucleic acid sequence of P2′ is immediately adjacent to the nucleic acid sequence of N. In certain embodiments, the nucleic acid sequence of P2′ is comprised within the nucleic acid sequence of N.

In certain embodiments, the nucleic acid sequence of P1′ is comprised within the nucleic acid sequence of A2. In certain embodiments, the nucleic acid sequence of P1′ is immediately adjacent to the nucleic acid sequence of A2. In certain embodiments, the nucleic acid sequence of P1′ is immediately adjacent to the nucleic acid sequence of N. In certain embodiments, the nucleic acid sequence of P1′ is comprised within the nucleic acid sequence of N.

In certain embodiments, the nucleic acid sequence of P2′ is comprised within the nucleic acid sequence of S1. In certain embodiments, the nucleic acid sequence of P2′ is immediately adjacent to the nucleic acid sequence of S 1. In certain embodiments, the nucleic acid sequence of P1′ is comprised within the nucleic acid sequence of S2. In certain embodiments, the nucleic acid sequence of P1′ is immediately adjacent to the nucleic acid sequence of S2.

Cargo

The donor template of the gene editing systems described herein comprises a cargo (N). The cargo may be of any length necessary in order to achieve the desired outcome. For example, a cargo sequence may be less than 2500 base pairs or less than 2500 nucleotides in length. In other embodiments, the cargo sequence may be 12 kb or less. In other embodiments, the cargo sequence may be 10 kb or less. In other embodiments, the cargo sequence may be 7 kb or less. In other embodiments, the cargo sequence may be 5 kb or less. In other embodiments, the cargo sequence may be 4 kb or less. In other embodiments, the cargo sequence may be 3 kb or less. In other embodiments, the cargo sequence may be 2 kb or less. In other embodiments, the cargo sequence may be 1 kb or less. In certain embodiments, the cargo can be between about 5-10 kb in length. In another embodiment, the cargo can be between about 1-5 kb in length. In another embodiment, the cargo can be between about 0-1 kb in length. For example, in exemplary embodiments, the cargo can be about 1000, 900, 800, 700, 600, 500, 400, 300, 200, or 100 base pairs or nucleotides in length. In other exemplary embodiments, the cargo can be about 100, 90, 80, 70, 60, 50, 40, 30, 20, 10, 9, 8, 7, 6, 5, 4, 3, 2, 1 or 0 base pairs or nucleotides in length. Those of skill in the art will readily ascertain that when the donor template is delivered using a delivery vehicle (e.g., a viral delivery vehicle such as an adeno-associated virus (AAV), adenovirus, lentivirus, integration-deficient lentivirus (IDLV), or herpes simplex virus (HSV) delivery vehicle) with size limitations, the size of the donor template, including cargo, should not exceed the size limitation of the delivery system.

In certain embodiments, the cargo comprises a replacement sequence. In certain embodiments, the cargo comprises an exon of a gene sequence. In certain embodiments, the cargo comprises an intron of a gene sequence. In certain embodiments, the cargo comprises a cDNA sequence. In certain embodiments, the cargo comprises a transcriptional regulatory element. In certain embodiments, the cargo comprises a reverse complement of a replacement sequence, an exon of a gene sequence, an intron of a gene sequence, a cDNA sequence or a transcriptional regulatory element. In certain embodiments, the cargo comprises a portion of a replacement sequence, an exon of a gene sequence, an intron of a gene sequence, a cDNA sequence or a transcriptional regulatory element. In certain embodiments, the cargo is a transgene sequence. In certain embodiments, the cargo introduces a deletion into a target nucleic acid. In certain embodiments, the cargo comprises an exogenous sequence. In other embodiments, the cargo comprises an endogenous sequence.

Replacement sequences in donor templates have been described elsewhere, including in Cotta-Ramusino et al. A replacement sequence can be any suitable length (including zero nucleotides, where the desired repair outcome is a deletion), and typically includes one, two, three or more sequence modifications relative to the naturally-occurring sequence within a cell in which editing is desired. One common sequence modification involves the alteration of the naturally-occurring sequence to repair a mutation that is related to a disease or condition of which treatment is desired.

Another common sequence modification involves the alteration of one or more sequences that are complementary to, or code for, the PAM sequence of the RNA-guided nuclease or the targeting domain of the gRNA(s) being used to generate an SSB or DSB, to reduce or eliminate repeated cleavage of the target site after the replacement sequence has been incorporated into the target site.

Specific cargo can be selected for a given application based on the cell type to be edited, the target nucleic acid, and the effect to be achieved.

For example, it may be desirable, in certain embodiments, to “knock in” a desired gene sequence at a selected chromosomal locus in a target cell. In such cases, the cargo can comprise the desired gene sequence. In certain embodiments, the gene sequence encodes a desired protein, e.g., an exogenous protein, an orthologous protein, or an endogenous protein, or a combination thereof.

In certain embodiments, the cargo can contain a wild-type sequence, or a sequence comprising one or more modifications with respect to a wild-type sequence. For example, in embodiments in which it is desirable to correct a mutation in a target gene in a cell, the cargo can be designed to restore the wild-type sequence to the target protein.

It may also be desirable, in other embodiments, to “knock out” a gene sequence at a selected chromosomal locus in the target cell. In such cases, the cargo can be designed to integrate at site that disrupts expression of the target gene sequence, for example, at a coding region of the target gene sequence, or at an expression control region for the target gene sequence, e.g., a promoter or enhancer of the target gene sequence. In other embodiments, the cargo can be designed to disrupt the target gene sequence. For example, in certain embodiments, the cargo can introduce a deletion, insertion, stop codon, or frameshift mutation into the target nucleic acid.

In certain embodiments, the donor is designed to delete all or a portion of the target nucleic acid sequence. In certain embodiments, the homology arms of the donor can be designed to flank the desired deletion site. In certain embodiments, the donor does not contain a cargo sequence between the homology arms, resulting in a deletion of the portion of the target nucleic acid positioned between the homology arms following targeted integration of the donor. In other embodiments, the donor contains a cargo sequence homologous to the target nucleic acid in which one or more nucleotides of the target nucleic acid sequence are absent from the cargo. Following targeted integration of the donor, the target nucleic acid will comprise a deletion at the residues absent from the cargo sequence. The size of the deletion can be selected based on the size of the target nucleic acid and the desired effect. In certain embodiments, the donor is designed to introduce a deletion of 1-2000 nucleotides in the target nucleic acid following targeted integration. In other embodiments, the donor is designed to introduce a deletion of 1-1000 nucleotides in the target nucleic acid following targeted integration. In other embodiments, the donor is designed to introduce a deletion of 1-500 nucleotides in the target nucleic acid following targeted integration. In other embodiments, the donor is designed to introduce a deletion of 1-100 nucleotides in the target nucleic acid following targeted integration. In exemplary embodiments, the donor is designed to introduce a deletion of about 2000, 1500, 1000, 900, 800, 700, 600, 500, 400, 300, 200, 100, 90, 80, 70, 60, 50, 40, 30, 20, 15, 10, 9, 8, 7, 6, 5, 4, 3, 2, or 1 nucleotides in the target nucleic acid following targeted integration. In other embodiments, the donor is designed to introduce a deletion of more than 2000 nucleotides from the target nucleic acid following targeted integration, for example, a deletion of about 2000, 3000, 4000, 5000, 6000, 7000, 8000, 9000, 10,000 nucleotides or more.

In certain embodiments, the cargo can comprise a promoter sequence. In other embodiments, the cargo is designed to integrate at a site that is under the control of a promoter endogenous to the target cell.

In certain embodiments, a cargo encoding an exogenous or orthologous protein or polypeptide can be integrated into a chromosomal sequence encoding a protein, such that the chromosomal sequence is inactivated, but the exogenous sequence is expressed. In other embodiments, the cargo sequence may be integrated into a chromosomal sequence without altering expression of a chromosomal sequence. This can be achieved by integrating the cargo at a “safe harbor” locus, such as the Rosa26 locus, HPRT locus, or AAV locus.

In certain embodiments, the cargo encodes a protein related to a disease or disorder. In certain embodiments, the cargo can encode a wild-type form of a protein, or is designed to restore expression of a wild-type form of a protein, where the protein is deficient in a subject afflicted with a disease or disorder. In other embodiments, the cargo encodes a protein related to a disease or disorder, where the protein encoded by the cargo comprises at least one modification, such that the altered version of the protein protects against the development of the disease or disorder. In other embodiments, the cargo encodes a protein comprising at least one modification, such that the altered version of the protein causes or potentiates a disease or disorder.

In certain embodiments, the cargo can be used to insert a gene from one species into the genome of a different species. For example, “humanized” animal models and/or “humanized” animal cells can be generated through targeted integration of human genes into the genome of a non-human animal species, e.g., mouse, rat, or non-human primate species. In certain embodiments, such humanized animal models and animal cells contain an integrated sequence encoding one or more human proteins.

In another embodiment, the cargo encodes a protein that confers a benefit on plant species, including crops such as grains, fruits, or vegetables. For example, the cargo can encode a protein that allows plants to be cultivated at higher temperatures, have a prolonged shelf life following harvest, or confer disease resistance. In certain embodiments, the cargo can encode a protein that confers resistance to diseases or pests (see, e.g., Jones et al. (1994) Science 266:789 (cloning of the tomato Cf-9 gene for resistance to Cladosporium fulvum); Martin et al. (1993) Science 262:1432; Mindrinos et al. (1994) Cell 78:1089 (RSP2 gene for resistance to Pseudomonas syringae); PCT International Patent Publication No. WO 96/30517 (resistance to soybean cyst nematode)). In other embodiments, the cargo can encode a protein that encodes resistance to an herbicide, as described in US2013/0326645A1, the entire contents of which are incorporated herein by reference. In another embodiment, the cargo encodes a protein that confers a value-added trait to a plant cell, for example and without limitation: modified fatty acid metabolism, decreased phytate content, and modified carbohydrate composition effected, e.g., by transforming plants with a gene encoding an enzyme that alters the branching pattern of starch (See, e.g., Shiroza et al. (1988) J. Bacteol. 170:810 (nucleotide sequence of Streptococcus mutant fructosyltransferase gene); Steinmetz et al. (1985) Mol. Gen. Genet. 20:220 (levansucrase gene); Pen et al. (1992) Bio/Technology 10:292 (α-amylase); Elliot et al. (1993) Plant Molec. Biol. 21:515 (nucleotide sequences of tomato invertase genes); Sogaard et al. (1993) J. Biol. Chem. 268:22480 (barley α-amylase gene); and Fisher et al. (1993) Plant Physiol. 102:1045 (maize endosperm starch branching enzyme II)). Other exemplary cargo useful for targeted integration in plant cells are described in US2013/0326645A1, the entire contents of which are incorporated herein by reference.

Additional cargo can be selected by the skilled artisan for a given application based on the cell type to be edited, the target nucleic acid, and the effect to be achieved.

Stuffers

In certain embodiments, the donor template may optionally comprise one or more stuffer sequences. Generally, a stuffer sequence is a heterologous or random nucleic acid sequence that has been selected to (a) facilitate (or to not inhibit) the targeted integration of a donor template of the present disclosure into a target site and the subsequent amplification of an amplicon comprising the stuffer sequence according to certain methods of this disclosure, but (b) to avoid driving integration of the donor template into another site. The stuffer sequence may be positioned, for instance, between a homology arm A1 and a primer site P2′ to adjust the size of the amplicon that will be generated when the donor template sequence is integrated into the target site. Such size adjustments may be employed, as one example, to balance the size of the amplicons produced by integrated and non-integrated target sites and, consequently to balance the efficiencies with which each amplicon is produced in a single PCR reaction; this in turn may facilitate the quantitative assessment of the rate of targeted integration based on the relative abundance of the two amplicons in a reaction mixture.

To facilitate targeted integration and amplification, the stuffer sequence may be selected to minimize the formation of secondary structures which may interfere with the resolution of the cleavage site by the DNA repair machinery (e.g., via homologous recombination) or which may interfere with amplification. In certain embodiments, the donor template comprises, from 5′ to 3′,

A1--S1--P2′--N--A2, or

A1--N--P1′--S2--A2;

wherein S1 is a first stuffer sequence and S2 is a second stuffer sequence.

In certain embodiments, the donor template comprises from 5′ to 3′,

A1--S1--P2′--N--P1′--S2--A2,

wherein S1 is a first stuffer sequence and S2 is a second stuffer sequence.

In certain embodiments, the stuffer sequence comprises about the same guanine-cytosine content (“GC content”) as the genome of the cell as a whole. In certain embodiments, the stuffer sequences comprise about the same GC content as the targeted locus. For example, when the target cell is a human cell, the stuffer sequence comprises about 40% GC content. In certain embodiments, a stuffer sequence may be designed by generating random nucleic acid sequence sequences comprising the desired GC content. For example, to generate a stuffer sequence comprising 40% GC content, nucleic acid sequences having the following distribution of nucleotides may be designed: A=30%, T=30%, G=20%, C=20%. Methods for determining the GC content of the genome or the GC content of the target locus are known to those of skill in the art. Thus, in certain embodiments, the stuffer sequence comprises 20%, 25%, 30%, 35%, 40%, 45%, 50%, 55% 60%, 65%, 70%, or 75% GC content. Exemplary 2.0 kilobase stuffer sequences having 40±5% GC content are provided herein as SEQ ID NOs: 23-123.

In certain embodiments, the first stuffer has a sequence comprising at least 5, at least 10, at least 15, at least 20, at least 25, at least 30, at least 35, at least 40, at least 45, at lest 50, at least 55, at least 60, at least 65, at least 70, at least 75, at least 80, at least 85, at least 90, at least 95, at least 100, at least 105, at least 110, at least 115, at least 120, at least 125, at least 130, at least 135, at least 140, at least 145, at least 150, at least 155, at least 160, at least 165, at least 170, at least 175, at least 180, at least 185, at least 190, at least 195, at least 200, at least 205, at least 210, at least 215, at least 220, at least 225, at least 230, at least 235, at least 240, at least 245, at least 250, at least 275, at least 300, at least 325, at least 350, at least 375, at least 400, at least 425, at least 450, at least 475, or at least 500 nucleotides of a sequence set forth in SEQ ID NOs: 23-123. In another embodiment, the second stuffer has a sequence comprising at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, at least 125, at least 130, at least 135, at least 140, at least 145, at least 150, at least 155, at least 160, at least 165, at least 170, at least 175, at least 180, at least 185, at least 190, at least 195, at least 200, at least 205, at least 210, at least 215, at least 220, at least 225, at least 230, at least 235, at least 240, at least 245, at least 250, at least 275, at least 300, at least 325, at least 350, at least 375, at least 400, at least 425, at least 450, at least 475, or at least 500 nucleotides of a sequence set forth in SEQ ID NOs: 23-123.

It is preferable that the stuffer sequence not interfere with the resolution of the cleavage site at the target nucleic acid. Thus, the stuffer sequence should have minimal sequence identity to the nucleic acid sequence at the cleavage site of the target nucleic acid. In certain embodiments, the stuffer sequence is less than 80%, 70%, 60%, 55%, 50%, 45%, 40%, 35%, 30%, 25%, 20%, or 10% identical to any nucleic acid sequence within 500, 450, 400, 350, 300, 250, 200, 150, 100, 50 nucleotides from the cleavage site of the target nucleic acid. In certain embodiments, the stuffer sequence is less than 80%, 70%, 60%, 55%, 50%, 45%, 40%, 35%, 30%, 25%, 20%, or 10% identical to any nucleic acid sequence within 500, 450, 400, 350, 300, 250, 200, 150, 100, 50 base pairs from the cleavage site of the target nucleic acid.

In order to avoid off-target molecular recombination events, it is preferable that the stuffer sequence have minimal homology to a nucleic acid sequence in the genome of the target cell. In certain embodiments, the stuffer sequence has minimal sequence identity to a nucleic acid in the genome of the target cell. In certain embodiments, the stuffer sequence is less than 80%, 70%, 60%, 55%, 50%, 45%, 40%, 35%, 30%, 25%, 20%, or 10% identical to any nucleic acid sequence of the same length (as measured in base pairs or nucleotides) in the genome of the target cell. In certain embodiments, a 20 base pair stretch of the stuffer sequence is less than 80%, 70%, 60%, 55%, 50%, 45%, 40%, 35%, 30%, 25%, 20%, or 10% identical to any at least 20 base pair stretch of nucleic acid of the target cell genome. In certain embodiments, a 20 nucleotide stretch of the stuffer sequence is less than 60%, 55%, 50%, 45%, 40%, 35%, 30%, 25%, 20%, or 10% identical to any at least 20 nucleotide stretch of nucleic acid of the target cell genome.

In certain embodiments, the stuffer sequence has minimal sequence identity to a nucleic acid sequence in the donor template (e.g., the nucleic acid sequence of the cargo, or the nucleic acid sequence of a priming site present in the donor template). In certain embodiments, the stuffer sequence is less than 80%, 70%, 60%, 55%, 50%, 45%, 40%, 35%, 30%, 25%, 20%, or 10% identical to any nucleic acid sequence of the same length (as measured in base pairs or nucleotides) in the donor template. In certain embodiments, a 20 base pair stretch of the stuffer sequence is less than 80%, 70%, 60%, 55%, 50%, 45%, 40%, 35%, 30%, 25%, 20%, or 10% identical to any 20 base pair stretch of nucleic acid of the donor template. In certain embodiments, a 20 nucleotide stretch of the stuffer sequence is less than 80%, 70%, 60%, 55%, 50%, 45%, 40%, 35%, 30%, 25%, 20%, or 10% identical to any 20 nucleotide stretch of nucleic acid of the donor template.

In certain embodiments, the length of the first homology arm and its adjacent stuffer sequence (i.e., A1+S1) is approximately equal to the length of the second homology arm and its adjacent stuffer sequence (i.e., A2+S2). For example, in certain embodiments the length of A1+S1 is the same as the length of A2+S2 (as determined in base pairs or nucleotides). In certain embodiments, the length of A1+S1 differs from the length of A2+S2 by 25 nucleotides or less. In certain embodiments, the length of A1+S1 differs from the length of A2+S2 by 24, 23, 22, 21, 20, 19 18, 17, 16, 15, 14, 13, 12, 11, 10, 9, 8, 7, 6, 5, 4, 3, or 2 nucleotides or less. In certain embodiments, the length of A1+S1 differs from the length of A2+S2 by 25 base pairs or less. In certain embodiments, the length of A1+S1 differs from the length of A2+S2 by 24, 23, 22, 21, 20, 19 18, 17, 16, 15, 14, 13, 12, 11, 10, 9, 8, 7, 6, 5, 4, 3, or 2 base pairs or less.

In certain embodiments, the length of A1+H1 is 250 base pairs or less. In certain embodiments, the length of A1+H1 is 200 base pairs or less. In certain embodiments, the length of A1+H1 is 150 base pairs or less. In certain embodiments, the length of A1+H1 is 100 base pairs or less. In certain embodiments, the length of A1+H1 is 50 base pairs or less. In certain embodiments, the length of A1+H1 is 250, 200, 190, 180, 170, 160, 150, 140, 130, 120, 110, 100, 90, 80, 70, 60, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, or 20 base pairs. In certain embodiments, the length of A1+H1 is 40 base pairs. In certain embodiments, the length of A2+H2 is 250 base pairs or less. In certain embodiments, the length of A2+H2 is 200 base pairs or less. In certain embodiments, the length of A2+H2 is 150 base pairs or less. In certain embodiments, the length of A2+H2 is 100 base pairs or less. In certain embodiments, the length of A2+H2 is 50 base pairs or less. In certain embodiments, the length of A2+H2 is 250, 200, 190, 180, 170, 160, 150, 140, 130, 120, 110, 100, 90, 80, 70, 60, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, or 20 base pairs. In certain embodiments, the length of A2+H2 is 40 base pairs.

In certain embodiments, the length of A1+S1 is the same as the length of H1+X+H2 (as determined in nucleotides or base pairs). In certain embodiments, the length of A1+S1 differs from the length of H1+X+H2 by less than 25 nucleotides. In certain embodiments, the length of A1+S1 differs from the length of H1+X+H2 by 24, 23, 22, 21, 20, 19 18, 17, 16, 15, 14, 13, 12, 11, 10, 9, 8, 7, 6, 5, 4, 3, or 2 nucleotides. In certain embodiments, the length of A1+S1 differs from the length of H1+X+H2 by less than 25 base pairs. In certain embodiments, the length of A1+S1 differs from the length of H1+X+H2 by 24, 23, 22, 21, 20, 19 18, 17, 16, 15, 14, 13, 12, 11, 10, 9, 8, 7, 6, 5, 4, 3, or 2 base pairs.

In certain embodiments, the length of A2+S2 is the same as the length of H1+X+H2 (as determined in nucleotides or base pairs). In certain embodiments, the length of A2+S2 differs from the length of H1+X+H2 by less than 25 nucleotides. In certain embodiments, the length of A2+S2 differs from the length of H1+X+H2 by 24, 23, 22, 21, 20, 19 18, 17, 16, 15, 14, 13, 12, 11, 10, 9, 8, 7, 6, 5, 4, 3, or 2 nucleotides. In certain embodiments, the length of A2+S2 differs from the length of H1+X+H2 by less than 25 base pairs. In certain embodiments, the length of A2+S2 differs from the length of H1+X+H2 by 24, 23, 22, 21, 20, 19 18, 17, 16, 15, 14, 13, 12, 11, 10, 9, 8, 7, 6, 5, 4, 3, or 2 base pairs.

### Target Cells

Genome editing systems according to this disclosure can be used to manipulate or modify a cell, e.g., to edit or modify a target nucleic acid. The manipulating can occur, in various embodiments, in vivo or ex vivo.

A variety of cell types can be manipulated or modified according to the embodiments of this disclosure, and in some cases, such as in vivo applications, a plurality of cell types are modified or manipulated, for example by delivering genome editing systems according to this disclosure to a plurality of cell types. In other cases, however, it may be desirable to limit manipulation or modification to a particular cell type or types. For instance, it can be desirable in some instances to edit a cell with limited differentiation potential or a terminally differentiated cell, such as a photoreceptor cell in the case of Maeder, in which modification of a genotype is expected to result in a change in cell phenotype. In other cases, however, it may be desirable to edit a less differentiated, multipotent or pluripotent, stem or progenitor cell. By way of example, the cell may be an embryonic stem cell, induced pluripotent stem cell (iPSC), hematopoietic stem/progenitor cell (HSPC), or other stem or progenitor cell type that differentiates into a cell type of relevance to a given application or indication

In certain embodiments, the cell being manipulated is a eukaryotic cell. For example, but not by way of limitation, the cell is a vertebrate, mammalian, rodent, goat, pig, bird, chicken, turkey, cow, horse, sheep, fish, primate, or human cell. In certain embodiments, the cell being manipulated is a somatic cell, a germ cell, or a prenatal cell. In certain embodiments, the cell being manipulated is a zygotic cell, a blastocyst cell, an embryonic cell, a stem cell, a mitotically competent cell, or a meiotically competent cell. In certain embodiments, the cell being manipulated is not part of a human embryo. In certain embodiments, the cell being manipulated is a T cell, a CD8⁺ T cell, a CD8⁺ naïve T cell, a CD4⁺ central memory T cell, a CD8⁺ central memory T cell, a CD4⁺ effector memory T cell, a CD4⁺ effector memory T cell, a CD4⁺ T cell, a CD4⁺ stem cell memory T cell, a CD8⁺ stem cell memory T cell, a CD4⁺ helper T cell, a regulatory T cell, a cytotoxic T cell, a natural killer T cell, a CD4+ naïve T cell, a TH17 CD4⁺ T cell, a TH1 CD4⁺ T cell, a TH2 CD4⁺ T cell, a TH9 CD4⁺ T cell, a CD4⁺ Foxp3⁺ T cell, a CD4⁺ CD25⁺ CD127⁻ T cell, a CD4⁺ CD25⁺ CD127⁻ Foxp3⁺ T cell. In certain embodiments, the cell being manipulated is a long term hematopoietic stem cell, a short term hematopoietic stem cell, a multipotent progenitor cell, a lineage restricted progenitor cell, a lymphoid progenitor cell, a myeloid progenitor cell, a common myeloid progenitor cell, an erythroid progenitor cell, a megakaryocyte erythroid progenitor cell, a retinal cell, a photoreceptor cell, a rod cell, a cone cell, a retinal pigmented epithelium cell, a trabecular meshwork cell, a cochlear hair cell, an outer hair cell, an inner hair cell, a pulmonary epithelial cell, a bronchial epithelial cell, an alveolar epithelial cell, a pulmonary epithelial progenitor cell, a striated muscle cell, a cardiac muscle cell, a muscle satellite cell, a neuron, a neuronal stem cell, a mesenchymal stem cell, an induced pluripotent stem (iPS) cell, an embryonic stem cell, a monocyte, a megakaryocyte, a neutrophil, an eosinophil, a basophil, a mast cell, a reticulocyte, a B cell, e.g., a progenitor B cell, a Pre B cell, a Pro B cell, a memory B cell, a plasma B cell, a gastrointestinal epithelial cell, a biliary epithelial cell, a pancreatic ductal epithelial cell, an intestinal stem cell, a hepatocyte, a liver stellate cell, a Kupffer cell, an osteoblast, an osteoclast, an adipocyte, a preadipocyte, a pancreatic islet cell (e.g., a beta cell, an alpha cell, a delta cell), a pancreatic exocrine cell, a Schwann cell, or an oligodendrocyte. In certain embodiments, the manipulated cell is a plant cell, e.g., a monocot or a dicot cell.

In certain embodiments, the target cell is a circulating blood cell, e.g., a reticulocyte, megakaryocyte erythroid progenitor (MEP) cell, myeloid progenitor cell (CMP/GMP), lymphoid progenitor (LP) cell, hematopoietic stem/progenitor cell (HSC), or endothelial cell (EC). In certain embodiments, the target cell is a bone marrow cell (e.g., a reticulocyte, an erythroid cell (e.g., erythroblast), an MEP cell, myeloid progenitor cell (CMP/GMP), LP cell, erythroid progenitor (EP) cell, HSC, multipotent progenitor (MPP) cell, endothelial cell (EC), hemogenic endothelial (HE) cell, or mesenchymal stem cell). In certain embodiments, the target cell is a myeloid progenitor cell (e.g., a common myeloid progenitor (CMP) cell or granulocyte macrophage progenitor (GMP) cell). In certain embodiments, the target cell is a lymphoid progenitor cell, e.g., a common lymphoid progenitor (CLP) cell. In certain embodiments, the target cell is an erythroid progenitor cell (e.g., an MEP cell). In certain embodiments, the target cell is a hematopoietic stem/progenitor cell (e.g., a long term HSC (LT-HSC), short term HSC (ST-HSC), MPP cell, or lineage restricted progenitor (LRP) cell). In certain embodiments, the target cell is a CD34⁺ cell, CD34⁺ CD90⁺ cell, CD34⁺ CD38⁻ cell, CD34⁺ CD90⁺ CD49rCD38⁻ CD45RA⁻ cell, CD105⁺ cell, CD31⁺, or CD133⁺ cell, or a CD34⁺ CD90⁺ CD133⁺ cell. In certain embodiments, the target cell is an umbilical cord blood CD34⁺ HSPC, umbilical cord venous endothelial cell, umbilical cord arterial endothelial cell, amniotic fluid CD34⁺ cell, amniotic fluid endothelial cell, placental endothelial cell, or placental hematopoietic CD34⁺ cell. In certain embodiments, the target cell is a mobilized peripheral blood hematopoietic CD34⁺ cell (after the patient is treated with a mobilization agent, e.g., G-CSF or Plerixafor). In certain embodiments, the target cell is a peripheral blood endothelial cell.

As a corollary, the cell being modified or manipulated is, variously, a dividing cell or a non-dividing cell, depending on the cell type(s) being targeted and/or the desired editing outcome.

When cells are manipulated or modified ex vivo, the cells can be used (e.g., administered to a subject) immediately, or they can be maintained or stored for later use. Those of skill in the art will appreciate that cells can be maintained in culture or stored (e.g., frozen in liquid nitrogen) using any suitable method known in the art.

### Implementation of Genome Editing Systems: Delivery, Formulations and Routes of Administration

As discussed above, the genome editing systems of this disclosure can be implemented in any suitable manner, meaning that the components of such systems, including without limitation the RNA-guided nuclease, gRNA, and optional donor template nucleic acid, can be delivered, formulated, or administered in any suitable form or combination of forms that results in the transduction, expression or introduction of a genome editing system and/or causes a desired repair outcome in a cell, tissue or subject. Tables 10 and 11 set forth several, non-limiting examples of genome editing system implementations. Those of skill in the art will appreciate, however, that these listings are not comprehensive, and that other implementations are possible. With reference to Table 10 in particular, the table lists several exemplary implementations of a genome editing system comprising a single gRNA and an optional donor template. However, genome editing systems according to this disclosure can incorporate multiple gRNAs, multiple RNA-guided nucleases, and other components such as proteins, and a variety of implementations will be evident to the skilled artisan based on the principles illustrated in the table. In the table, [N/A] indicates that the genome editing system does not include the indicated component.

TABLE 10
Genome Editing System Components

| RNA- | | | |
|---|---|---|---|
| guided | | Donor | |
| Nuclease | gRNA | Template | Comments |
| Protein | RNA | [N/A] | An RNA-guided nuclease protein |
| | | | complexed with a gRNA molecule |
| | | | (an RNP complex) |
| Protein | RNA | DNA | An RNP complex as described above |
| | | | plus a single-stranded or double |
| | | | stranded donor template. |
| Protein | DNA | [N/A] | An RNA-guided nuclease protein plus |
| | | | gRNA transcribed from DNA. |
| Protein | DNA | DNA | An RNA-guided nuclease protein |
| | | | plus gRNA-encoding DNA and a |
| | | | separate DNA donor template. |
| Protein | DNA | | An RNA-guided nuclease protein and |
| | | | a single DNA encoding both a gRNA |
| | | | and a donor template. |
| DNA | | | A DNA or DNA vector encoding an |
| | | | RNA-guided nuclease, a gRNA and a |
| | | | donor template. |
| DNA | DNA | [N/A] | Two separate DNAs, or two separate |
| | | | DNA vectors, encoding the RNA- |
| | | | guided nuclease and the gRNA, |
| | | | respectively. |
| DNA | DNA | DNA | Three separate DNAs, or three |
| | | | separate DNA vectors, encoding the |
| | | | RNA-guided nuclease, the gRNA and |
| | | | the donor template, respectively. |
| DNA | | [N/A] | A DNA or DNA vector encoding an |
| | | | RNA-guided nuclease and a gRNA |
| DNA | | DNA | A first DNA or DNA vector encoding |
| | | | an RNA-guided nuclease and a gRNA, |
| | | | and a second DNA or DNA vector |
| | | | encoding a donor template. |
| DNA | DNA | | A first DNA or DNA vector encoding |
| | | | an RNA-guided nuclease and second |
| | | | DNA or DNA vector encoding a |
| | | | gRNA and a donor template. |
| | DNA | | A first DNA or DNA vector encoding |
| | DNA | | an RNA-guided nuclease and a donor |
| | | | template, and a second DNA or DNA |
| | | | vector encoding a gRNA |
| | DNA | | A DNA or DNA vector encoding an |
| | RNA | | RNA-guided nuclease and a donor |
| | | | template, and a gRNA |

| | | |
|---|---|---|
| RNA | [N/A] | An RNA or RNA vector encoding an |
| | | RNA-guided nuclease and comprising |
| | | a gRNA |
| RNA | DNA | An RNA or RNA vector encoding an |
| | | RNA-guided nuclease and comprising |
| | | a gRNA, and a DNA or DNA vector |
| | | encoding a donor template. |

Table 11 summarizes various delivery methods for the components of genome editing systems, as described herein. Again, the listing is intended to be exemplary rather than limiting.

TABLE 11

| | | Delivery | | | |
|---|---|---|---|---|---|
| | | into Non- | | | Type of |
| | | Dividing | Duration of | Genome | Molecule |
| Delivery Vector/Mode | | Cells | Expression | Integration | Delivered |
| Physical (e.g., electroporation, | | YES | Transient | NO | Nucleic Acids |
| particle gun, Calcium | | | | | and Proteins |
| Phosphate transfection, cell | | | | | |
| compression or squeezing) | | | | | |
| Viral | Retrovirus | NO | Stable | YES | RNA |
| | Lentivirus | YES | Stable | YES/NO with | RNA |
| | | | | modifications | |
| | Adenovirus | YES | Transient | NO | DNA |
| | Adeno- | YES | Stable | NO | DNA |
| | Associated Virus | | | | |
| | (AAV) | | | | |
| | Vaccinia Virus | YES | Very | NO | DNA |
| | | | Transient | | |
| | Herpes Simplex | YES | Stable | NO | DNA |
| | Virus | | | | |
| Non-Viral | Cationic | YES | Transient | Depends on | Nucleic Acids |
| | Liposomes | | | what is | and Proteins |
| | | | | delivered | |
| | Polymeric | YES | Transient | Depends on | Nucleic Acids |
| | Nanoparticles | | | what is | and Proteins |
| | | | | delivered | |
| Biological | Attenuated | YES | Transient | NO | Nucleic Acids |
| Non-Viral | Bacteria | | | | |
| Delivery | Engineered | YES | Transient | NO | Nucleic Acids |
| Vehicles | Bacteriophages | | | | |
| | Mammalian | YES | Transient | NO | Nucleic Acids |
| | Virus-like | | | | |
| | Particles | | | | |
| | Biological | YES | Transient | NO | Nucleic Acids |
| | liposomes: | | | | |
| | Erythrocyte | | | | |
| | Ghosts and | | | | |
| | Exosomes | | | | |

Nucleic Acid-Based Delivery of Genome Editing Systems

Nucleic acids encoding the various elements of a genome editing system according to the present disclosure can be administered to subjects or delivered into cells by art-known methods or as described herein. For example, RNA-guided nuclease-encoding and/or gRNA-encoding DNA, as well as donor template nucleic acids can be delivered by, e.g., vectors (e.g., viral or non-viral vectors), non-vector based methods (e.g., using naked DNA or DNA complexes), or a combination thereof.

Nucleic acids encoding genome editing systems or components thereof can be delivered directly to cells as naked DNA or RNA, for instance by means of transfection or electroporation, or can be conjugated to molecules (e.g., N-acetylgalactosamine) promoting uptake by the target cells (e.g., erythrocytes, HSCs). Nucleic acid vectors, such as the vectors summarized in Table 11, can also be used.

Nucleic acid vectors can comprise one or more sequences encoding genome editing system components, such as an RNA-guided nuclease, a gRNA and/or a donor template. A vector can also comprise a sequence encoding a signal peptide (e.g., for nuclear localization, nucleolar localization, or mitochondrial localization), associated with (e.g., inserted into or fused to) a sequence coding for a protein. As one example, a nucleic acid vectors can include a Cpf1 coding sequence that includes one or more nuclear localization sequences (e.g., a nuclear localization sequence from SV40).

The nucleic acid vector can also include any suitable number of regulatory/control elements, e.g., promoters, enhancers, introns, polyadenylation signals, Kozak consensus sequences, or internal ribosome entry sites (IRES). These elements are well known in the art, and are described in Cotta-Ramusino et al.

Nucleic acid vectors according to this disclosure include recombinant viral vectors. Exemplary viral vectors are set forth in Table 11, and additional suitable viral vectors and their use and production are described in Cotta-Ramusino et al. Other viral vectors known in the art can also be used. In addition, viral particles can be used to deliver genome editing system components in nucleic acid and/or peptide form. For example, “empty” viral particles can be assembled to contain any suitable cargo. Viral vectors and viral particles can also be engineered to incorporate targeting ligands to alter target tissue specificity.

In addition to viral vectors, non-viral vectors can be used to deliver nucleic acids encoding genome editing systems according to the present disclosure. One important category of non-viral nucleic acid vectors are nanoparticles, which can be organic or inorganic. Nanoparticles are well known in the art, and are summarized in Cotta-Ramusino et al. Any suitable nanoparticle design can be used to deliver genome editing system components or nucleic acids encoding such components. For instance, organic (e.g., lipid and/or polymer) nanoparticles can be suitable for use as delivery vehicles in certain embodiments of this disclosure. Exemplary lipids for use in nanoparticle formulations, and/or gene transfer are shown in Table 12, and Table 13 lists exemplary polymers for use in gene transfer and/or nanoparticle formulations.

TABLE 12
Lipids Used for Gene Transfer

| | Abbre- | |
|---|---|---|
| Lipid | viation | Feature |
| 1,2-Dioleoyl-sn-glycero-3-phosphatidylcholine | DOPC | Helper |
| 1,2-Dioleoyl-sn-glycero-3- | DOPE | Helper |
| phosphatidylethanolamine | | |
| Cholesterol | | Helper |
| N-[1-(2,3-Dioleyloxy)propyl]N,N,N- | DOTMA | Cationic |
| trimethylammonium chloride | | |
| 1,2-Dioleoyloxy-3-trimethylammonium-propane | DOTAP | Cationic |
| Dioctadecylamidoglycylspermine | DOGS | Cationic |
| N-(3-Aminopropyl)-N,N-dimethyl-2,3- | GAP- | Cationic |
| bis(dodecyloxy)-1-propanaminium bromide | DLRIE | |
| Cetyltrimethylammonium bromide | CTAB | Cationic |
| 6-Lauroxyhexyl ornithinate | LHON | Cationic |
| 1-(2,3-Dioleoyloxypropyl)-2,4,6- | 2Oc | Cationic |
| trimethylpyridinium | | |
| 2,3-Dioleyloxy-N-[2(sperminecarboxamido- | DOSPA | Cationic |
| ethyl]-N,N-dimethyl-1- | | |
| propanaminium trifluoroacetate | | |
| 1,2-Dioleyl-3-trimethylammonium-propane | DOPA | Cationic |
| N-(2-Hydroxyethyl)-N,N-dimethyl-2,3- | MDRIE | Cationic |
| bis(tetradecyloxy)-1-propanaminium bromide | | |
| Dimyristooxypropyl dimethyl hydroxyethyl | DMRI | Cationic |
| ammonium bromide | | |
| 3β-[N-(N′,N′-Dimethylaminoethane)- | DC-Chol | Cationic |
| carbamoyl]cholesterol | | |
| Bis-guanidium-tren-cholesterol | BGTC | Cationic |
| 1,3-Diodeoxy-2-(6-carboxy-spermyl)-propylamide | DOSPER | Cationic |
| Dimethyloctadecylammonium bromide | DDAB | Cationic |
| Dioctadecylamidoglicylspermidin | DSL | Cationic |
| rac-[(2,3-Dioctadecyloxypropyl)(2- | CLIP-1 | Cationic |
| hydroxyethyl)]-dimethylammonium chloride | | |
| rac-[2(2,3-Dihexadecyloxypropyl- | CLIP-6 | Cationic |
| oxymethyloxy)ethyl]trimethylammonium bromide | | |
| Ethyldimyristoylphosphatidylcholine | EDMPC | Cationic |
| 1,2-Distearyloxy-N,N-dimethyl-3-aminopropane | DSDMA | Cationic |
| 1,2-Dimyristoyl-trimethylammonium propane | DMTAP | Cationic |
| O,O′-Dimyristyl-N-lysyl aspartate | DMKE | Cationic |
| 1,2-Distearoyl-sn-glycero-3-ethylphosphocholine | DSEPC | Cationic |
| N-Palmitoyl D-erythro-sphingosyl | CCS | Cationic |
| carbamoyl-spermine | | |
| N-t-Butyl-N0-tetradecyl-3- | diC14- | Cationic |
| tetradecylaminopropionamidine | amidine | |
| Octadecenolyoxy[ethyl-2-heptadecenyl- | DOTIM | Cationic |
| 3 hydroxyethyl]imidazolinium chloride | | |
| N1-Cholesteryloxycarbonyl-3,7-diazanonane- | CDAN | Cationic |
| 1,9-diamine | | |
| 2-(3-[Bis(3-amino-propyl)-amino]propylamino)- | RPR209120 | Cationic |
| N-ditetradecylcarbamoylme-ethyl-acetamide | | |
| 1,2-dilinoleyloxy-3-dimethylaminopropane | DLinDMA | Cationic |
| 2,2-dilinoleyl-4-dimethylaminoethyl- | DLin- | Cationic |
| [1,3]-dioxolane | KC2-DMA | |
| dilinoleyl-methyl-4-dimethylaminobutyrate | DLin- | Cationic |
| | MC3-DMA | |

TABLE 13
Polymers Used for Gene Transfer

| Polymer | Abbreviation |
|---|---|
| Poly(ethylene)glycol | PEG |
| Polyethylenimine | PEI |
| Dithiobis(succinimidylpropionate) | DSP |
| Dimethyl-3,3′-dithiobispropionimidate | DTBP |
| Poly(ethylene imine) biscarbamate | PEIC |
| Poly(L-lysine) | PLL |
| Histidine modified PLL | |
| Poly(N-vinylpyrrolidone) | PVP |
| Poly(propylenimine) | PPI |
| Poly(amidoamine) | PAMAM |
| Poly(amido ethylenimine) | SS-PAEI |
| Triethylenetetramine | TETA |
| Poly(β-aminoester) | |
| Poly(4-hydroxy-L-proline ester) | PHP |
| Poly(allylamine) | |
| Poly(α-[4-aminobutyl]-L-glycolic acid) | PAGA |
| Poly(D,L-lactic-co-glycolic acid) | PLGA |
| Poly(N-ethyl-4-vinylpyridinium bromide) | |
| Poly(phosphazene)s | PPZ |
| Poly(phosphoester)s | PPE |
| Poly(phosphoramidate)s | PPA |
| Poly(N-2-hydroxypropylmethacrylamide) | pHPMA |
| Poly (2-(dimethylamino)ethyl methacrylate) | pDMAEMA |
| Poly(2-aminoethyl propylene phosphate) | PPE-EA |
| Chitosan | |
| Galactosylated chitosan | |
| N-Dodacylated chitosan | |
| Histone | |
| Collagen | |
| Dextran-spermine | D-SPM |

Non-viral vectors optionally include targeting modifications to improve uptake and/or selectively target certain cell types. These targeting modifications can include e.g., cell specific antigens, monoclonal antibodies, single chain antibodies, aptamers, polymers, sugars (e.g., N-acetylgalactosamine (GalNAc)), and cell penetrating peptides. Such vectors also optionally use fusogenic and endosome-destabilizing peptides/polymers, undergo acid-triggered conformational changes (e.g., to accelerate endosomal escape of the cargo), and/or incorporate a stimuli-cleavable polymer, e.g., for release in a cellular compartment. For example, disulfide-based cationic polymers that are cleaved in the reducing cellular environment can be used.

In certain embodiments, one or more nucleic acid molecules (e.g., DNA molecules) other than the components of a genome editing system, e.g., the RNA-guided nuclease component and/or the gRNA component described herein, are delivered. In certain embodiments, the nucleic acid molecule is delivered at the same time as one or more of the components of the Genome editing system. In certain embodiments, the nucleic acid molecule is delivered before or after (e.g., less than about 30 minutes, 1 hour, 2 hours, 3 hours, 6 hours, 9 hours, 12 hours, 1 day, 2 days, 3 days, 1 week, 2 weeks, or 4 weeks) one or more of the components of the Genome editing system are delivered. In certain embodiments, the nucleic acid molecule is delivered by a different means than one or more of the components of the genome editing system, e.g., the RNA-guided nuclease component and/or the gRNA component, are delivered. The nucleic acid molecule can be delivered by any of the delivery methods described herein. For example, the nucleic acid molecule can be delivered by a viral vector, e.g., an integration-deficient lentivirus, and the RNA-guided nuclease molecule component and/or the gRNA component can be delivered by electroporation, e.g., such that the toxicity caused by nucleic acids (e.g., DNAs) can be reduced. In certain embodiments, the nucleic acid molecule encodes a therapeutic protein, e.g., a protein described herein. In certain embodiments, the nucleic acid molecule encodes an RNA molecule, e.g., an RNA molecule described herein.

Delivery of RNPs and/or RNA Encoding Genome Editing System Components

RNPs (complexes of gRNAs and RNA-guided nucleases) and/or RNAs encoding RNA-guided nucleases and/or gRNAs, can be delivered into cells or administered to subjects by art-known methods, some of which are described in Cotta-Ramusino et al. In vitro, RNA-guided nuclease-encoding and/or gRNA-encoding RNA can be delivered, e.g., by microinjection, electroporation, transient cell compression or squeezing (see, e.g., Lee 2012). Lipid-mediated transfection, peptide-mediated delivery, GalNAc- or other conjugate-mediated delivery, and combinations thereof, can also be used for delivery in vitro and in vivo.

In vitro, delivery via electroporation comprises mixing the cells with the RNA encoding RNA-guided nucleases and/or gRNAs, with or without donor template nucleic acid molecules, in a cartridge, chamber or cuvette and applying one or more electrical impulses of defined duration and amplitude. Systems and protocols for electroporation are known in the art, and any suitable electroporation tool and/or protocol can be used in connection with the various embodiments of this disclosure. Exemplary systems include, but are not limited to, Nucleofector™ technologies (Lonza), Gene Pulser Xcell™ (BioRad), Flow Electroporation™ transfection systems (MaxCyte) and the Neon™ transfection systems (ThermoFisher).

Route of Administration

Genome editing systems, or cells modified or manipulated using such systems, can be administered to subjects by any suitable mode or route, whether local or systemic. Systemic modes of administration include oral and parenteral routes. Parenteral routes include, by way of example, intravenous, intramarrow, intrarterial, intramuscular, intradermal, subcutaneous, intranasal, and intraperitoneal routes. Components administered systemically can be modified or formulated to target, e.g., HSCs, hematopoietic stem/progenitor cells, or erythroid progenitors or precursor cells.

Local modes of administration include, by way of example, intramarrow injection into the trabecular bone or intrafemoral injection into the marrow space, and infusion into the portal vein. In certain embodiments, significantly smaller amounts of the components (compared with systemic approaches) can exert an effect when administered locally (for example, directly into the bone marrow) compared to when administered systemically (for example, intravenously). Local modes of administration can reduce or eliminate the incidence of potentially toxic side effects that may occur when therapeutically effective amounts of a component are administered systemically.

Administration can be provided as a periodic bolus (for example, intravenously) or as continuous infusion from an internal reservoir or from an external reservoir (for example, from an intravenous bag or implantable pump). Components can be administered locally, for example, by continuous release from a sustained release drug delivery device.

In addition, components can be formulated to permit release over a prolonged period of time. A release system can include a matrix of a biodegradable material or a material which releases the incorporated components by diffusion. The components can be homogeneously or heterogeneously distributed within the release system. A variety of release systems can be useful; however, the choice of the appropriate system will depend upon rate of release required by a particular application. Both non-degradable and degradable release systems can be used. Suitable release systems include polymers and polymeric matrices, non-polymeric matrices, or inorganic and organic excipients and diluents such as, but not limited to, calcium carbonate and sugar (for example, trehalose). Release systems may be natural or synthetic. However, synthetic release systems are preferred because generally they are more reliable, more reproducible and produce more defined release profiles. The release system material can be selected so that components having different molecular weights are released by diffusion through or degradation of the material.

Representative synthetic, biodegradable polymers include, for example: polyamides such as poly(amino acids) and poly(peptides); polyesters such as poly(lactic acid), poly(glycolic acid), poly(lactic-co-glycolic acid), and poly(caprolactone); poly(anhydrides); polyorthoesters; polycarbonates; and chemical derivatives thereof (substitutions, additions of chemical groups, for example, alkyl, alkylene, hydroxylations, oxidations, and other modifications routinely made by those skilled in the art), copolymers and mixtures thereof. Representative synthetic, non-degradable polymers include, for example: polyethers such as poly(ethylene oxide), poly(ethylene glycol), and poly(tetramethylene oxide); vinyl polymers-polyacrylates and polymethacrylates such as methyl, ethyl, other alkyl, hydroxyethyl methacrylate, acrylic and methacrylic acids, and others such as poly(vinyl alcohol), poly(vinyl pyrolidone), and poly(vinyl acetate); poly(urethanes); cellulose and its derivatives such as alkyl, hydroxyalkyl, ethers, esters, nitrocellulose, and various cellulose acetates; polysiloxanes; and any chemical derivatives thereof (substitutions, additions of chemical groups, for example, alkyl, alkylene, hydroxylations, oxidations, and other modifications routinely made by those skilled in the art), copolymers and mixtures thereof.

Poly(lactide-co-glycolide) microsphere can also be used. Typically, the microspheres are composed of a polymer of lactic acid and glycolic acid, which are structured to form hollow spheres. The spheres can be approximately 15-30 microns in diameter and can be loaded with components described herein.

Multi-Modal or Differential Delivery of Components

Skilled artisans will appreciate, in view of the instant disclosure, that different components of genome editing systems disclosed herein can be delivered together or separately and simultaneously or non-simultaneously. Separate and/or asynchronous delivery of genome editing system components can be particularly desirable to provide temporal or spatial control over the function of genome editing systems and to limit certain effects caused by their activity.

Different or differential modes as used herein refer to modes of delivery that confer different pharmacodynamic or pharmacokinetic properties on the subject component molecule, e.g., a RNA-guided nuclease molecule, gRNA, template nucleic acid, or payload. For example, the modes of delivery can result in different tissue distribution, different half-life, or different temporal distribution, e.g., in a selected compartment, tissue, or organ.

Some modes of delivery, e.g., delivery by a nucleic acid vector that persists in a cell, or in progeny of a cell, e.g., by autonomous replication or insertion into cellular nucleic acid, result in more persistent expression of and presence of a component. Examples include viral, e.g., AAV or lentivirus, delivery.

By way of example, the components of a genome editing system, e.g., a RNA-guided nuclease and a gRNA, can be delivered by modes that differ in terms of resulting half-life or persistent of the delivered component the body, or in a particular compartment, tissue or organ. In certain embodiments, a gRNA can be delivered by such modes. The RNA-guided nuclease molecule component can be delivered by a mode which results in less persistence or less exposure to the body or a particular compartment or tissue or organ.

More generally, in certain embodiments, a first mode of delivery is used to deliver a first component and a second mode of delivery is used to deliver a second component. The first mode of delivery confers a first pharmacodynamic or pharmacokinetic property. The first pharmacodynamic property can be, e.g., distribution, persistence, or exposure, of the component, or of a nucleic acid that encodes the component, in the body, a compartment, tissue or organ. The second mode of delivery confers a second pharmacodynamic or pharmacokinetic property. The second pharmacodynamic property can be, e.g., distribution, persistence, or exposure, of the component, or of a nucleic acid that encodes the component, in the body, a compartment, tissue or organ.

In certain embodiments, the first pharmacodynamic or pharmacokinetic property, e.g., distribution, persistence or exposure, is more limited than the second pharmacodynamic or pharmacokinetic property.

In certain embodiments, the first mode of delivery is selected to optimize, e.g., minimize, a pharmacodynamic or pharmacokinetic property, e.g., distribution, persistence or exposure.

In certain embodiments, the second mode of delivery is selected to optimize, e.g., maximize, a pharmacodynamic or pharmacokinetic property, e.g., distribution, persistence or exposure.

In certain embodiments, the first mode of delivery comprises the use of a relatively persistent element, e.g., a nucleic acid, e.g., a plasmid or viral vector, e.g., an AAV or lentivirus. As such vectors are relatively persistent product transcribed from them would be relatively persistent.

In certain embodiments, the second mode of delivery comprises a relatively transient element, e.g., an RNA or protein.

In certain embodiments, the first component comprises gRNA, and the delivery mode is relatively persistent, e.g., the gRNA is transcribed from a plasmid or viral vector, e.g., an AAV or lentivirus. Transcription of these genes would be of little physiological consequence because the genes do not encode for a protein product, and the gRNAs are incapable of acting in isolation. The second component, a RNA-guided nuclease molecule, is delivered in a transient manner, for example as mRNA or as protein, ensuring that the full RNA-guided nuclease molecule/gRNA complex is only present and active for a short period of time.

Furthermore, the components can be delivered in different molecular form or with different delivery vectors that complement one another to enhance safety and tissue specificity.

Use of differential delivery modes can enhance performance, safety, and/or efficacy, e.g., the likelihood of an eventual off-target modification can be reduced. Delivery of immunogenic components, e.g., Cas9 molecules, by less persistent modes can reduce immunogenicity, as peptides from the bacterially-derived Cas enzyme are displayed on the surface of the cell by MEW molecules. A two-part delivery system can alleviate these drawbacks.

Differential delivery modes can be used to deliver components to different, but overlapping target regions. The formation active complex is minimized outside the overlap of the target regions. Thus, in certain embodiments, a first component, e.g., a gRNA is delivered by a first delivery mode that results in a first spatial, e.g., tissue, distribution. A second component, e.g., a RNA-guided nuclease molecule is delivered by a second delivery mode that results in a second spatial, e.g., tissue, distribution. In certain embodiments, the first mode comprises a first element selected from a liposome, nanoparticle, e.g., polymeric nanoparticle, and a nucleic acid, e.g., viral vector. The second mode comprises a second element selected from the group. In certain embodiments, the first mode of delivery comprises a first targeting element, e.g., a cell specific receptor or an antibody, and the second mode of delivery does not include that element. In certain embodiments, the second mode of delivery comprises a second targeting element, e.g., a second cell specific receptor or second antibody.

When the RNA-guided nuclease molecule is delivered in a virus delivery vector, a liposome, or polymeric nanoparticle, there is the potential for delivery to and therapeutic activity in multiple tissues, when it may be desirable to only target a single tissue. A two-part delivery system can resolve this challenge and enhance tissue specificity. If the gRNA and the RNA-guided nuclease molecule are packaged in separated delivery vehicles with distinct but overlapping tissue tropism, the fully functional complex is only formed in the tissue that is targeted by both vectors.

### Exemplary Non-Limiting Embodiments

A. In certain non-limiting embodiments, the presently disclosed subject matter provides an isolated CRISPR from Prevotella and Franciscella 1 (Cpf1) RNA-guided nuclease comprising a nuclear localization signal (NLS).

A1. The foregoing Cpf1 RNA-guided nuclease of A, wherein the Cpf1 RNA-guided nuclease comprises an NLS at or near the N-terminus of the nuclease.

A2. The foregoing Cpf1 RNA-guided nuclease of A, wherein the Cpf1 RNA-guided nuclease comprises an NLS at or near the C-terminus of the nuclease.

A3. The foregoing Cpf1 RNA-guided nuclease of A1, where the Cpf1 RNA-guided nuclease comprises two NLS sequences at or near the N-terminus of the nuclease.

A4. The foregoing Cpf1 RNA-guided nuclease of A2, where the Cpf1 RNA-guided nuclease comprises two NLS sequences at or near the C-terminus of the nuclease.

A5. The foregoing Cpf1 RNA-guided nuclease of A, wherein the Cpf1 RNA-guided nuclease comprises an NLS at or near both the N-terminus and C-terminus of the nuclease.

A6. The foregoing Cpf1 RNA-guided nuclease of A, wherein if the Cpf1 RNA-guided nuclease comprises more than one NLS sequence, the NLS sequences are the same or different.

A7. The foregoing Cpf1 RNA-guided nuclease of A, wherein the NLS sequence or sequences are selected from the group consisting of: the nucleoplasmin NLS (nNLS) (SEQ ID NO: 1) and the simian virus 40 “SV40” NLS (sNLS) (SEQ ID NO: 2).

A8. The foregoing Cpf1 RNA-guided nuclease of A, wherein the sequence of the Cpf1 RNA-guided nuclease is selected from the group consisting: His-AsCpf1-nNLS (SEQ ID NO: 3); His-AsCpf1-sNLS (SEQ ID NO: 4; His-AsCpf1-sNLS-sNLS (SEQ ID NO: 5); His-sNLS-AsCpf1 (SEQ ID NO: 6); His-sNLS-sNLS-AsCpf1 (SEQ ID NO: 7); sNLS-sNLS-AsCpf1 (SEQ ID NO: 8); His-sNLS-AsCpf1-sNLS (SEQ ID NO: 9); and His-sNLS-sNLS-AsCpf1-sNLS-sNLS (SEQ ID NO: 10).

B. In certain non-limiting embodiments, the presently disclosed subject matter provides an isolated Cpf1 RNA-guided nuclease comprising a deletion or substitution of a cysteine amino acid.

B1. The foregoing Cpf1 RNA-guided nuclease of B, wherein the Cpf1 RNA-guided nuclease comprises a deletion or substitution at C65, C205, C334, C379, C608, C674, C1025, or C1248 of the wild type AsCpf1 amino acid sequence.

B2. The foregoing Cpf1 RNA-guided nuclease of B1, wherein the Cpf1 RNA-guided nuclease comprises a substitution selected from the group consisting of C65S/A, C205S/A, C334S/A, C379S/A, C608S/A, C674S/A, and C1025S/A relative to the wild type AsCpf1 amino acid sequence.

B3. The foregoing Cpf1 RNA-guided nuclease of B1, wherein the Cpf1 RNA-guided nuclease comprises a deletion or substitution at either C334 and C674 or C334, C379, and C674 of the wild type AsCpf1 amino acid sequence.

B4. The foregoing Cpf1 RNA-guided nuclease of B3, wherein the Cpf1 RNA-guided nuclease comprises a substitution selected from the group consisting of: (1) C334S/A and C674S/A; and (2) C334S/A, C379S/A, and C674S/A relative to the wild type AsCpf1 amino acid sequence

B5. The foregoing Cpf1 RNA-guided nuclease of B, wherein the Cpf1 RNA-guided nuclease further comprises an NLS.

B6. The foregoing Cpf1 RNA-guided nuclease of B5, wherein the sequence of the Cpf1 RNA-guided nuclease is selected from His-AsCpf1-nNLS Cys-less (SEQ ID NO: 11) and His-AsCpf1-nNLS Cys-low (SEQ ID NO: 12)

C. In certain embodiments, the presently disclosed subject matter provides for an isolated nucleic acid encoding a foregoing Cpf1 RNA-guided nuclease of any of A-A8 and B-B8.

D. In certain embodiments, the presently disclosed subject matter provides for a genome editing system, the genome editing system comprising:

a guide RNA (gRNA); and

a foregoing Cpf1 RNA-guided nuclease of any of A-A8 and B-B8 or encoded by a foregoing nucleic acid of C.

E. In certain embodiments, the presently disclosed subject matter provides for a method of modifying a target sequence of interest in a cell, comprising contacting the cell with:

a gRNA complementary with a target sequence of interest; and

a foregoing Cpf1 RNA-guided nuclease of any of A-A8 and B-B8 or encoded by a foregoing nucleic acid of C,

wherein said Cpf1 RNA-guided nuclease modifies the target sequence of interest.

E1. The foregoing method of E, wherein the cell is a T cell, a hematopoietic stem cell (HSC), or a human umbilical cord blood-derived erythroid progenitor cell (HUDEP cell).

E2. The foregoing method of E1, wherein the HSC is a CD34+ cell, CD34+CD90+ cell, CD34+CD38− cell, CD34+CD90+CD49f+CD38−CD45RA− cell, CD105+ cell, CD31+, or CD133+ cell, or a CD34+CD90+CD133+ cell.

E3. The foregoing method of E1, wherein the T cell is a CD8⁺ T cell, a CD8⁺ naïve T cell, a CD4⁺ central memory T cell, a CD8⁺ central memory T cell, a CD4⁺ effector memory T cell, a CD4⁺ effector memory T cell, a CD4⁺ T cell, a CD4⁺ stem cell memory T cell, a CD8⁺ stem cell memory T cell, a CD4⁺ helper T cell, a regulatory T cell, a cytotoxic T cell, a natural killer T cell, a CD4+ naïve T cell, a TH17 CD4⁺ T cell, a TH1 CD4⁺ T cell, a TH2 CD4⁺ T cell, a TH9 CD4⁺ T cell, a CD4⁺ Foxp3⁺ T cell, a CD4⁺ CD25⁺ CD127⁻ T cell or a CD4⁺ CD25⁺ CD127⁻ Foxp3⁺ T cell.

E4. The foregoing method of E, wherein the Cpf1 RNA-guided nuclease modifies the target sequence of interest to achieve at least 20%, 30%, 40%, 50%, 60%, 70%, 80%, or 90% editing.

E5. The foregoing method of E, further comprising a second gRNA complementary with a second target sequence of interest.

E6. The foregoing method of E, further comprising a second RNA-guided nuclease.

E7. The foregoing method of E, wherein the target sequence of interest is selected from the group consisting of: a portion of the HBG1 gene sequence; and a portion of the BCL11a gene sequence.

E8. The foregoing method of E7, wherein the portion of the HBG1 gene sequence is the −110 nt promoter region of the HBG gene.

E9. The foregoing method of E8, wherein the portion of the HBG1 gene sequence is the CAAT box of the −110 nt promoter region of the HBG gene.

E10. The foregoing method of E7, wherein the portion of the Bcl11a gene sequence is the +58 DHS region of intron 2 of the BCL11a gene.

E11. The foregoing method of E10, wherein the portion of the Bcl11a gene sequence is GATA1 motif of the +58 DHS region of intron 2 of the BCL11a gene.

E12. The foregoing method of E, wherein the target sequence of interest is selected from the group consisting of: a portion of the FAS gene sequence; a portion of the BID gene sequence; a portion of the CTLA4 gene sequence; a portion of the PDCD1 gene sequence; a portion of the CBLB gene sequence; a portion of the PTPN6 gene sequence; a portion of the B2M gene sequence; a portion of the TRAC gene sequence; and a portion of the TRBC gene sequence.

E13. The foregoing method of E12, wherein the target sequence of interest is selected from the group consisting of: a portion of the B2M gene sequence; a portion of the TRAC gene sequence; and a portion of the TRBC gene sequence.

E14. The foregoing method of E13, wherein the portion of the B2M gene sequence is within the first 500 bp of the coding sequence of the B2M gene.

E15. The foregoing method of E13, wherein the portion of the B2M gene sequence is between the 501st nucleotide and the last nucleotide of the coding sequence of the B2M gene.

E16. The foregoing cell of E12, wherein the portion of the TRAC gene sequence is within the first 500 bp of the coding sequence of the TRAC gene.

E17. The foregoing cell of E12, wherein the portion of the TRBC gene sequence is within the first 500 bp of the coding sequence of the TRBC gene.

F. In certain embodiments, the presently disclosed subject matter provides for a method of treating a subject, comprising contacting a cell from a subject with:

a gRNA complementary to a target sequence of a target nucleic acid; and

a foregoing Cpf1 RNA-guided nuclease of any of A-A8 and B-B8.

F1. The foregoing method of F, wherein the Cpf1 molecule forms a double strand break in the target nucleic acid.

F2. The foregoing method of F or F1, wherein the Cpf1 molecule is a selected from the group consisting of Acidaminococcus sp. strain BV3L6 Cpf1 molecule (AsCpf1), Lachnospiraceae bacterium ND2006 Cpf1 molecule (LbCpf1), and Lachnospiraceae bacterium MA2020 (Lb2Cpf1).

F3. The foregoing method of any one of F-F2, wherein the subject suffers from a hemoglobinopathy.

F4. The foregoing method of F3, wherein the hemoglobinopathy is sickle cell disease or beta-thalassemia.

F5. The foregoing method of any one of F-F4, wherein the cell is a T cell, a hematopoietic stem cell (HSC), or a human umbilical cord blood-derived erythroid progenitor cell (HUDEP cell).

F6. The foregoing method of F5, wherein the T cell is a CD8⁺ T cell, a CD8⁺ naïve T cell, a CD4⁺ central memory T cell, a CD8⁺ central memory T cell, a CD4⁺ effector memory T cell, a CD4⁺ effector memory T cell, a CD4⁺ T cell, a CD4⁺ stem cell memory T cell, a CD8⁺ stem cell memory T cell, a CD4⁺ helper T cell, a regulatory T cell, a cytotoxic T cell, a natural killer T cell, a CD4+ naïve T cell, a TH17 CD4⁺ T cell, a TH1 CD4⁺ T cell, a TH2 CD4⁺ T cell, a TH9 CD4⁺ T cell, a CD4⁺ Foxp3⁺ T cell, a CD4⁺ CD25⁺ CD127⁻ T cell or a CD4⁺ CD25⁺ CD127⁻ Foxp3⁺ T cell.

F7. The foregoing method of F5, wherein the HSC cell is CD34⁺ cell, CD34⁺ CD90⁺ cell, CD34⁺ CD38⁻ cell, CD34⁺ CD90⁺ CD49f⁺CD38⁻ CD45RA⁻ cell, CD105⁺ cell, CD31⁺, or CD133⁺ cell, or a CD34⁺ CD90⁺ CD133⁺ cell.

F8. The foregoing method of any one of F-F7, wherein the contacting is performed ex vivo.

F9. The foregoing method of any one of F-F8, wherein the contacted cell is returned to the subject's body.

G. In certain embodiments, the presently disclosed subject matter provides for a reaction mixture comprising:

(a) a foregoing Cpf1 RNA-guided nuclease of any of A-A8 and B-B8,

(b) a gRNA complementary to a target sequence of a target nucleic acid, and

(c) a cell from a subject who would benefit from one or more modifications of the target nucleic acid.

H. In certain embodiments, the presently disclosed subject matter provides for a kit comprising:

(a) a foregoing Cpf1 RNA-guided nuclease of any one of A-A8 and B-B8, or a nucleic acid composition that encodes the Cpf1 RNA-guided nuclease, and

(b) a gRNA complementary to a target sequence of a target nucleic acid or a nucleic acid composition the gRNA.

I. In certain embodiments, the presently disclosed subject matter provides for a cell comprising a modification in a target nucleic acid sequence introduced via the foregoing genome editing system of D.

I1. The foregoing cell of I, wherein the modification is to the HBG1 gene sequence or the Bcl11a gene sequence.

I2. The foregoing cell of I1, wherein the modified HBG1 gene sequence is the −110 nt promoter region of the HBG gene.

I3. The foregoing cell of claim I1, wherein the modified HBG1 gene sequence is the CAAT box of the −110 nt promoter region of the HBG gene.

I4. The foregoing cell of claim I1, wherein the modified Bcl11a gene sequence is the +58 DHS region of intron 2 of the BCL11a gene.

I5. The foregoing cell of claim I1, wherein the modified Bcl11a gene sequence is GATA1 motif of the +58 DHS region of intron 2 of the BCL11a gene.

J. In certain embodiments, the presently disclosed subject matter provides for a method of evaluating CRISPR/Cpf1-mediated editing of a target nucleic acid sequence and/or modulation of expression of a target nucleic acid sequence by a test Cpf1 RNA-guided nuclease comprising:

(a) determining the activity of the test Cpf1 RNA-guided nuclease with respect to the editing and/or modulation of expression of a target nucleic acid sequence comprising a matched site target nucleic acid sequence;

(b) comparing the activity of the test Cpf1 RNA-guided nuclease to the activity of a control RNA-guided nuclease with respect to the editing and/or modulation of expression of the target nucleic acid sequence comprising the matched site target nucleic acid sequence.

J1. The foregoing method of J, wherein the matched side target nucleic acid sequence is selected from the group consisting of: Matched Site 1 (SEQ ID NO: 13), Matched Site 5 (SEQ ID NO: 14), Matched Site 11 (SEQ ID NO: 15), and Matched Site 18 (SEQ ID NO: 16).

J2. The foregoing method of J wherein the test Cpf1 RNA-guided nuclease and the control RNA-guided nuclease:

(a) have the same amino acid sequence; and

(b) are assayed for activity in distinct cell types.

J3. The foregoing method of J, wherein the test Cpf1 RNA-guided nuclease and the control RNA-guided nuclease:

(a) have the same amino acid sequence; and

(b) are assayed for activity in distinct formulations.

J4. The foregoing method of J, wherein the test Cpf1 RNA-guided nuclease and the control RNA-guided nuclease:

(a) have the same amino acid sequence; and
(b) are assayed for activity at distinct concentrations.

J5. The foregoing method of J, wherein the test Cpf1 RNA-guided nuclease and the control RNA-guided nuclease:

(a) have the same amino acid sequence; and
(b) are assayed for activity after having been manufactured via distinct processes.

J6. The foregoing method of J, wherein the test Cpf1 RNA-guided nuclease and the control RNA-guided nuclease:

(a) have the same amino acid sequence; and

(b) are assayed for activity after having been delivered to a cell via distinct processes.

J7. The foregoing method of J, wherein the test Cpf1 RNA-guided nuclease and the control RNA-guided nuclease comprise distinct amino acid sequences.

K. In certain embodiments, the presently disclosed subject matter provides for a cell comprising a CRISPR system capable of downregulating gene expression of an endogenous gene selected from the group consisting of BC11a and HBG1.

K1. The foregoing cell of K, wherein the CRISPR system comprises a gRNA complementary to a portion of the BC11a gene sequence.

K2. The foregoing cell of K1, wherein the portion of the BC11a gene sequence is the +58 DHS region of intron 2 of the BCL11a gene.

K3. The foregoing cell of K1, wherein the portion of the BC11a gene sequence is the GATA1 motif of the +58 DHS region of intron 2 of the BCL11a gene.

K4. The foregoing cell of K, wherein the CRISPR system comprises a gRNA complementary to a portion of the HBG1 gene sequence.

K5. The foregoing cell of K4, wherein the portion of the HBG1 gene sequence is the −110 nt promoter region of the HBG1 gene.

K6. The foregoing cell of K4, wherein the portion of the HBG1 gene sequence is the CAAT box of the −110 nt promoter region of the HBG1 gene.

K7. The foregoing cell of K, wherein the cell is a CD34+ cell, CD34+CD90+ cell, CD34+CD38− cell, CD34+CD90+CD49f+CD38−CD45RA− cell, CD105+cell, CD31+, or CD133+ cell, or a CD34+CD90+CD133+ cell.

L. In certain embodiments, the presently disclosed subject matter provides for a cell comprising a CRISPR system capable of downregulating gene expression of at least one endogenous gene selected from the group consisting of FAS, BID, CTLA4, PDCD1, CBLB, PTPN6, B2M, TRAC, CIITA and TRBC.

L1. The foregoing cell of L, wherein the CRISPR system comprises a gRNA complementary to a portion of the B2M gene sequence.

L2. The foregoing cell of L1, wherein the portion of the B2M gene sequence is within the first 500 bp of the coding sequence of the B2M gene.

L3. The foregoing cell of L1, wherein the portion of the B2M gene sequence is between the 501st nucleotide and the last nucleotide of the coding sequence of the B2M gene.

L4. The foregoing cell of any of L-L3, wherein the CRISPR system comprises a gRNA complementary to a portion of the TRAC gene sequence.

L5. The foregoing cell of L4, wherein the portion of the TRAC gene sequence is within the first 500 bp of the coding sequence of the TRAC gene.

L6. The foregoing cell of any of L-L5, wherein the CRISPR system comprises a gRNA complementary to a portion of the TRBC gene sequence.

L7. The foregoing cell of L6, wherein the portion of the TRBC gene sequence is within the first 500 bp of the coding sequence of the TRBC gene.

L8. The foregoing cell of any of L-L7, wherein the CRISPR system comprises a gRNA complementary to a portion of the CIITA gene sequence.

L9. The foregoing cell of L8, wherein the portion of the CIITA gene sequence is within the first 500 bp of the coding sequence of the CIITA gene.

L10. The foregoing cell of L, wherein the CRISPR system is capable of downregulating gene expression of the group consisting of B2M, TRAC, and CIITA.

L11. The foregoing cell of L, wherein the CRISPR system is capable of downregulating gene expression of the group consisting of B2M, TRAC, TRBC, and CIITA.

L12. The foregoing cell of any one of L-L11, wherein the cell is a CD8⁺ T cell, a CD8⁺ naïve T cell, a CD4⁺ central memory T cell, a CD8⁺ central memory T cell, a CD4⁺ effector memory T cell, a CD4⁺ effector memory T cell, a CD4⁺ T cell, a CD4⁺ stem cell memory T cell, a CD8⁺ stem cell memory T cell, a CD4⁺ helper T cell, a regulatory T cell, a cytotoxic T cell, a natural killer T cell, a CD4+ naïve T cell, a TH17 CD4⁺ T cell, a TH1 CD4⁺ T cell, a TH2 CD4⁺ T cell, a TH9 CD4⁺ T cell, a CD4⁺ Foxp3⁺ T cell, a CD4⁺ CD25⁺ CD127⁻ T cell or a CD4⁺ CD25⁺ CD127⁻ Foxp3⁺ T cell.

M. In certain embodiments, the presently disclosed subject matter provides for an assay for evaluating CRISPR/Cpf1-mediated editing of a target nucleic acid sequence and/or modulation of expression of a target nucleic acid sequence by a test Cpf1 RNA-guided nuclease comprising:

(a) determining the activity of the test Cpf1 RNA-guided nuclease with respect to the editing and/or modulation of expression of a target nucleic acid sequence comprising a matched site target nucleic acid sequence;

(b) comparing the activity of the test Cpf1 RNA-guided nuclease to the activity of a control RNA-guided nuclease with respect to the editing and/or modulation of expression of the target nucleic acid sequence comprising the matched site target nucleic acid sequence.

M1. The foregoing assay of M wherein the matched side target nucleic acid sequence is selected from the group consisting of: Matched Site 1 (SEQ ID NO: 13), Matched Site 5 (SEQ ID NO: 14), Matched Site 11 (SEQ ID NO: 15), and Matched Site 18 (SEQ ID NO: 16).

M2. The foregoing assay of M1 wherein the test Cpf1 RNA-guided nuclease and the control RNA-guided nuclease:

(a) have the same amino acid sequence; and

(b) are assayed for activity in distinct cell types.

M3. The foregoing assay of M2 wherein the test Cpf1 RNA-guided nuclease and the control RNA-guided nuclease:

(a) have the same amino acid sequence; and

(b) are assayed for activity in distinct formulations.

M4. The foregoing assay of M wherein the test Cpf1 RNA-guided nuclease and the control RNA-guided nuclease:

(a) have the same amino acid sequence; and

(b) are assayed for activity at distinct concentrations.

M5. The foregoing assay of M wherein the test Cpf1 RNA-guided nuclease and the control RNA-guided nuclease:

(a) have the same amino acid sequence; and

(b) are assayed for activity after having been manufactured via distinct processes.

M6. The foregoing assay of M wherein the test Cpf1 RNA-guided nuclease and the control RNA-guided nuclease:

(a) have the same amino acid sequence; and

(b) are assayed for activity after having been delivered to a cell via distinct processes.

N. In certain embodiments, the presently disclosed subject matter provides for a multiplex genome editing system, the multiplex genome editing system comprising:

a first guide RNA (gRNA) comprising a first targeting domain that is complementary to a target sequence of first gene;

a second gRNA molecule comprising a second targeting domain that is complementary to a target sequence of a second gene; and

a foregoing Cpf1 RNA-guided nuclease of any of A-A8 and B-B8 or encoded by a foregoing nucleic acid of C.

N1. The foregoing multiplex genome editing system of N, wherein the first gene and the second gene are selected from the group consisting of B2M, TRAC, CIITA and TRBC.

N2. The foregoing multiplex genome editing system of N further comprising: a third gRNA molecule comprising a third targeting domain that is complementary to a target sequence of a third gene.

N3. The foregoing multiplex genome editing system of N2, wherein the first gene, the second gene and the third gene are selected from the group consisting of B2M, TRAC, CIITA and TRBC.

N4. The foregoing multiplex genome editing system of N2 further comprising: a fourth gRNA molecule comprising a fourth targeting domain that is complementary to a target sequence of a fourth gene.

N5. The foregoing multiplex genome editing system of N4, wherein the first gene, the second gene, the third gene and the fourth gene are selected from the group consisting of B2M, TRAC, CIITA and TRBC.

O. In certain embodiments, the presently disclosed subject matter provides for a method of modifying multiple genes in a cell, comprising contacting the cell with:

a first (gRNA) comprising a first targeting domain that is complementary to a target sequence of first gene;

a second gRNA molecule comprising a second targeting domain that is complementary to a target sequence of a second gene; and

a foregoing Cpf1 RNA-guided nuclease of any of A-A8 and B-B8 or encoded by a foregoing nucleic acid of C,

wherein said Cpf1 RNA-guided nuclease modifies the first gene and the second gene.

O1. The foregoing method of O further comprising: a third gRNA molecule comprising a third targeting domain that is complementary to a target sequence of a third gene, wherein said Cpf1 RNA-guided nuclease modifies the first gene, the second gene and the third gene.

O2. The foregoing method of O1 further comprising: a fourth gRNA molecule comprising a fourth targeting domain that is complementary to a target sequence of a fourth gene, wherein said Cpf1 RNA-guided nuclease modifies the first gene, the second gene, the third gene and the fourth gene.

O3. The foregoing method of O2, wherein the first gene, the second gene, the third gene and the fourth gene are selected from the group consisting of B2M, TRAC, CIITA and TRBC genes.

O4. The foregoing method of O, wherein the cell is a T cell.

### EXAMPLES

The following Examples are merely illustrative and are not intended to limit the scope or content of the invention in any way.

### Example 1—Efficient Editing of Adult Human CD34+ Cells with S. pyogenes Cas9 and AsCpf1 Variants as Evaluated by a Benchmarking Assay Using Matched Sites

CRISPR/Cpf1-mediated editing of a target nucleic acid sequence and/or modulation of expression of a target nucleic acid sequence can be evaluated by comparing the activity of a test CRISPR/Cpf1 editing system to a control CRISPR/RNA-guided nuclease editing system with respect to a target nucleic acid sequence, e.g., a “matched site” target nucleic acid sequence.

As matched site target nucleic acid sequences incorporate both the requirements to be edited by Cpf1 as well as a second RNA-guided nuclease, e.g., Cas9. For example, the TTTV AsCpf1 wild type protospacer adjacent motif (“PAM”) and a NGG SpCas9 wild type PAM were employed in the instant example. As noted above, the test Cpf1 protein can comprise one or more modifications relative to the wild type Cpf1 protein.

Examples of such modifications include, but are not limited to, the aforementioned modifications to incorporate one or more NLS sequence, to incorporate a six-histidine purification sequence, and the alteration of a Cpf1 protein cysteine amino acid, as well as combinations thereof.

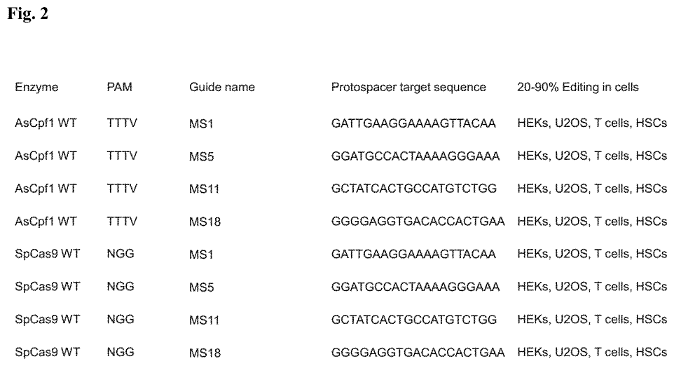

Exemplary matched site target nucleic acid sequences that were employed in the instant example include Matched Site 1 (“MS1”; SEQ ID NO: 13), Matched Site 5 (“MS5”; SEQ ID NO: 14), Matched Site 11 (“MS11”; SEQ ID NO: 15), and Matched Site 18 (“MS18”; SEQ ID NO: 18) (FIG. 2).

To evaluate CRISPR/Cpf1-mediated versus CRISPR/Cas9-mediated editing of a target nucleic acid sequence and/or modulation of expression of a target nucleic acid sequence in a particular cell type, e.g. CD34⁺ HSCs, a CRISPR/Cpf1 genome editing system, i.e., a system comprising a Cpf1 RNA-guided nuclease and a gRNA complementary to at least a portion of a target nucleic acid comprising a matched site target, is introduced, e.g., as an RNP or via the use of a vector coding for the components of the system, into the cell of the cell type of interest. The editing of the target nucleic acid sequence and/or modulation of expression of a target nucleic acid sequence is detected as disclosed herein. The detected editing of the target nucleic acid sequence and/or modulation of expression of a target nucleic acid sequence is compared to the editing of the target nucleic acid sequence and/or modulation of expression of a target nucleic acid sequence detected when a CRISPR/Cas9 genome editing system is employed with the same matched site target and the same cell type.

The above-described method of comparing CRISPR/Cpf1-mediated versus CRISPR/Cas9-mediated editing (or editing by another CRISPR-based system) of a target nucleic acid sequence and/or modulation of expression of a target nucleic acid sequence allows for an evaluation of particular attributes of the CRISPR/Cpf1-mediated editing system employed. For example, but not by way of limitation, such methods can be used to evaluate CRISPR/Cpf1-mediated versus CRISPR/Cas9-mediated editing of a target nucleic acid sequence and/or modulation of expression of a target nucleic acid sequence to identify differences in activity of Cpf1 RNA-guided nucleases and/or gRNAs prepared by distinct manufacturing process. Such methods can also identify differences in activity of Cpf1 RNA-guided nucleases and/or gRNAs present in distinct formulations as well as those employing distinct delivery strategies.

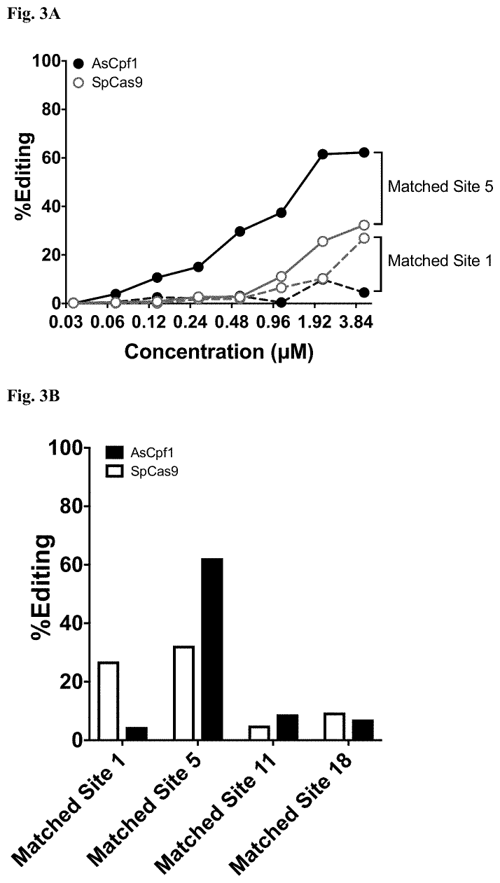

In this example, the baseline level of editing of wild type (WT) S. pyogenes (Sp) Cas9 and AsCpf1 nuclease were compared in adult human mobilized peripheral blood CD34⁺ hematopoietic stem/progenitor cells. These CD34⁺ cells are clinical targets for the treatment of hematologic disorders (e.g., β-hemoglobinopathies), where a diseased phenotype can be corrected by a nuclease modified genotype. To determine baseline editing by SpCas9 and AsCpf1 in CD34⁺ cells, the cells were thawed and pre-stimulated in cytokines and then electroporated with AsCpf1 or SpCas9 protein complexed to guide RNAs targeting the matched sites (MS) in the human genome (FIG. 2). The term ‘matched site’ refers to the fact that the site targeted by the nuclease is the same for both AsCpf1 and SpCas9, despite their utilization of different PAM sequences (NGG and TTTV, respectively). To determine the minimal effective concentration of ribonucleoprotein (RNP) required for efficient editing in CD34⁺ cells, RNP dose responses were performed in CD34⁺ cells for several matched sites, two of which are depicted in FIG. 3A. To determine percentage of editing at the target sites, genomic (g)DNA was extracted from AsCpf1 or SpCas9 electroporated cells, amplicon PCR performed on the target sites, followed by DNA sequencing analysis.

FIG. 3A depicts the results where, in one instance, AsCpf1 is substantially more efficient than SpCas9 for editing the same target site (MS5) and one instance in which SpCas9 is more efficient at editing the same target site compared to AsCpf1 (MS1). The gRNA used to target MS5 was the MS5 guide RNA. In this example, ˜4 μM Cpf1 RNP supported efficient (˜60%) editing at Matched Site 5 and the editing was higher compared to editing achieved with the same dose of SpCas9 RNP targeting that site (FIG. 3A).

FIG. 3B depicts the results when multiple matched sites were compared after electroporation with 4.4 μM RNP. These results establish that editing is occurring at sites in which: a) SpCas9 is more efficient than AsCpf1, b) AsCpf1 is more efficient than SpCas9, and c) the levels of editing are similar between SpCas9 and AsCpf1.

To determine the optimal protein configuration for editing in CD34⁺ cells, AsCpf1 proteins were synthesized containing different types of NLS sequences that were located at different locations, e.g., the C-terminus or N-terminus, of the AsCpf1 protein. As described herein, nNLS represents the nucleoplasmin NLS, and sNLS refers to the SV40 NLS (FIG. 4). The following NLS configurations were analyzed in this example, His-AsCpf1-nNLS (SEQ ID NO: 3), His-sNLS-sNLS-AsCpf1 (SEQ ID NO: 7), His-sNLS-AsCpf1 (SEQ ID NO: 6), His-sNLS-AsCpf1-sNLS (SEQ ID NO: 9), His-AsCpf1-sNLS-sNLS (SEQ ID NO: 5) and His-AsCpf1-sNLS (SEQ ID NO: 4). The different protein variants were complexed to MS5 gRNA and then electroporated into CD34⁺ cells, T cells, and HUDEPs (4.4 μM RNP). In FIG. 4, the results are depicted % editing normalized to the variant displaying maximal editing for each cell type. Together, these data show that different species of nucleases have variable activity at the same target site in CD34⁺ cells (among other cells) and that efficient editing by AsCpf1 can be achieved in CD34⁺ cells (among other cells). In particular, as shown in FIG. 4, the protein variants with the following NLS configurations His-sNLS-sNLS-AsCpf1, His-sNLS-AsCpf1 and His-AsCpf1-sNLS-sNLS exhibited high editing across all cell types at MS5.

### Example 2—Electroporation Pulse Code Screening

In order to identify electroporation pulse codes allowing for higher efficiency editing by the Cpf1 RNA-guided nucleases of the present disclosure, a screen of possible pulse codes was performed. FIG. 18 depicts nucleofection screening for AsCpf1 in HUDEPs. The dose was 2.2 μM AsCpf1 RNP using matched site 5 guide RNA, at 2:1 guide:protein. AsCpf1 WT protein had endotoxin levels <5 EU/mL. Lonza solutions SE, SF, and SG were tested with 50,000 HUDEPs/condition using different pulse programs. Pulse codes CA-137 and CA-138 with solution SE demonstrated optimal editing.

FIG. 19 depicts nucleofection screening for AsCpf1 in HSCs. The dose was 2.2 uM AsCpf1 RNP using matched site 5 (MS5) guide RNA, at 2:1 guide:protein. The AsCpf1 WT protein had endotoxin levels <5 EU/mL. Lonza solutions P1, P2, P3, P4, and P5 were tested with 50,000 HSCs/condition using different pulse programs. Pulse codes CA-137 (also referred to herein as “Condition 2”) and CA-138 with solution P2 demonstrated optimal editing, as well as FF-100 and FF-104.

FIG. 20 confirms the increased efficiency of a pulse code identified in the above-described screens. Specifically, FIG. 20 depicts the use of a particular pulse code in Lonza Amaxa increases editing at the BCL11a locus in HSCs using various gRNAs and PAM variants. The dose was 4.4 μM RNP for all guides, with 2:1 guide:protein ratio. 50,000 HSCs were treated per condition. AsCpf1 WT, RR, and RVR proteins had endotoxin levels of <5 EU/mL.

### Example 3—AsCpf1 Directed Editing of CD34⁺ Cells at Target Sites in the Human Genome that are Associated with Increased Production of Fetal Hemoglobin

Fetal hemoglobin (HbF) expression can be induced through targeted disruption of the erythroid cell specific expression of a transcriptional repressor, BCL11A (Canvers et al., Nature, 527(12): 192-197). One potential strategy to increase HbF expression through a gene editing strategy is to direct Cpf1 to disrupt the GATA1 binding motif in the erythroid specific enhancer of the BCL11A gene that is in the +58 DHS region of intron 2 of the BCL11A gene. In the example, AsCpf1 mediated editing of target sites in the +58 DHS region of intron 2 of the BCL11A gene were evaluated.

First, AsCpf1 variant guide RNAs with different PAMs (FIG. 1) were screened in HUDEP2 cells and then the most efficient guide RNAs and nuclease variants were tested in mPB CD34⁺ cells (FIG. 17). The sequences of the guide RNAs tested in FIG. 17 are provided in FIG. 7. In particular, FIG. 17 depicts screening of the BCL11a enhancer region with AsCpf1 WT and RR and RVR PAM variants along with one WT FnCpf1 target in HUDEPs and HSCs. The HUDEP screen was performed with the CA-137 pulse program and Lonza solution SE. The HSC screen was performed with the pulse code EO-100 and Lonza solution P3. The control guide for BCL11a (named KOBEH in FIG. 17) is shown as well. The dose was 4.4 uM RNP for all guides, with 2:1 guide:protein ratio. Approximately 50,000 HSCs were treated per condition. The AsCpf1 WT, RR, and RVR proteins had endotoxin levels of <5 EU/mL.).

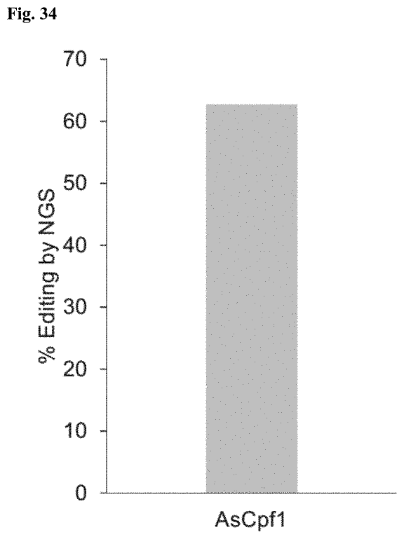

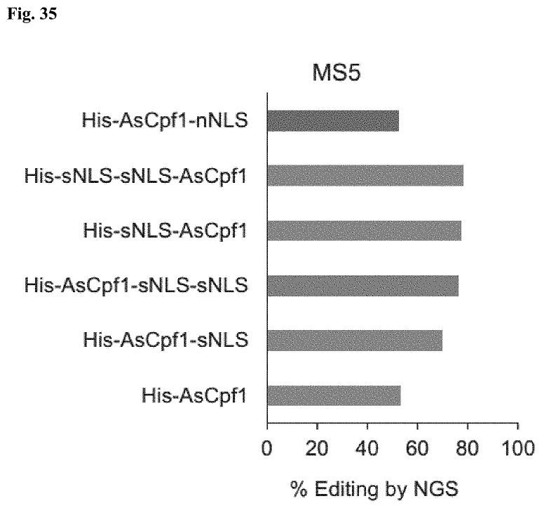

Another potential strategy to increase HbF expression is through targeted disruption of the HBG genes, e.g., HBG1 or HBG2. FIG. 16 depicts the targeting of the HBG1 promoter region with AsCpf1 WT and RR PAM variant in HUDEPs and HSCs. The sequences of the guide RNAs tested in FIG. 16 are provided in FIG. 6. Moraxella bovoculi AAX11_00205 (Mb3Cpf1) was also tested, which is referred to as MbCpf1 in FIG. 6. The HUDEP experiment was performed with the CA-137 pulse program and Lonza solution SE. The HSC screen was performed with pulse code EO-100 and Lonza solution P3. The dose was 4.4 uM RNP for all guides, with 2:1 guide:protein ratio. Approximately 50,000 HSCs were treated per condition. The AsCpf1 WT and RR proteins had endotoxin levels of <5 EU/mL. FIG. 34 depicts the editing of the HBG1 locus using the HBG1-1 gRNA. AsCpf1 was complexed with gRNA at a 1:4 protein:guide ratio for a final RNP dose of 8 uM in cells. The RNPs were incubated for 30 mins at RT for complexation. As shown in FIG. 34, the use of the HBG1-1 gRNA resulted in greater than 60% editing in HSCs. The differences between the editing efficiencies represented in FIG. 16 and FIG. 34 are reflective of the different conditions under which the experiments were performed, e.g., such as electroporation pulse code.

Together, these data show efficient editing by AsCpf1 variants in CD34⁺ cells at clinically relevant loci (i.e., known HPFH target sites).

### Example 4—Generation of Cysteine-Modified Cpf1 Proteins and RNPs

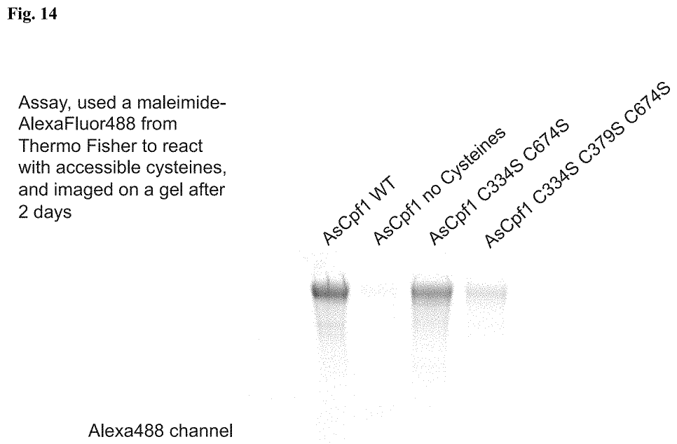

Because disulfide bond formation is known to promote protein aggregation, the Cpf1 crystal structure and the known Cpf1 primary amino acid sequence were analyzed in an effort to identify cysteines that could be altered to reduce the possibility of disulfide bond formation (FIG. 13). Of the eight cysteines present in Cpf1, several appeared to be solvent exposed while others appeared to be buried and inaccessible to other intermolecular cysteines and therefore not a high risk for disulfide bond formation (FIG. 13). A cysteine labeling assay with AlexaFluor 488 C5 maleimide (Part # A10254 ThermoFisher Scientific) was employed to demonstrate significantly reduced accessibility of cysteine residues in AsCpf1 C334S C379S C674S after 48 hours of incubation as compared to wild type and a variant where residue C379 is not mutated to serine (FIG. 14). The “AsCpf1 no Cysteines” sample shows no labeling with maleimide reagent. AsCpf1 C334S C674S sample, the variant which is not mutated at C379, shows labeling nearly equivalent to wild type, indicating that C379, which appears partially exposed in the crystal structure, is readily accessible to AlexaFluor 488 C5 maleimide reagent. All labeling reactions were performed according to manufacturer's recommendations. Briefly, this requires a 20-fold molar excess of AlexaFluor 488 C5 maleimide dye with 10 μM protein, incubated at 4° C. for a minimum of 24 hours in H150 buffer and 10% DMSO.

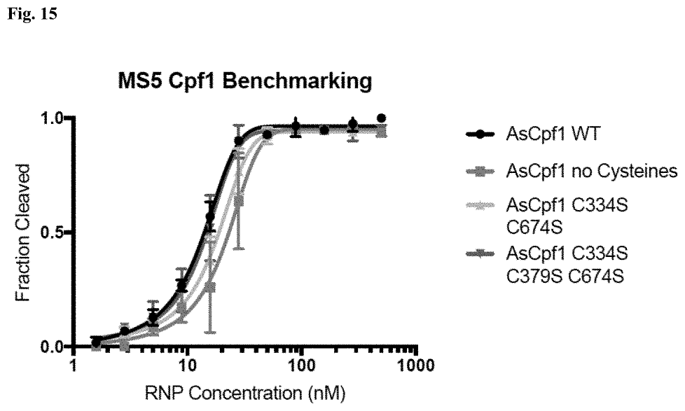

The editing capability of the wild type and three variants described above were compared in FIG. 15. While a reduction in editing is observed with the Cys-less AsCpf1, the AsCpf1-C334S-C674S and AsCpf1-C334S-C379S-C674S variants achieved levels of editing similar to that of the AsCpf1 wild type (FIG. 15).

### Example 5—Highly Efficient Editing with CRISPR-Cpf1 in Primary T Cells

Introduction

The CRISPR-Cpf1 (Cas12a) system can offer several potential advantages over other nucleases for ex vivo genome editing therapies, including a smaller single crRNA that can be readily synthesized, the ability to target T- and C-rich PAMs with the wild-type protein and engineered PAM variants, and a 5′-staggered cut which may lead to different repair outcomes.

For ex vivo delivery, the use of ribonucleoprotein (RNP) complexes can be preferable, in many instances, to nucleic acid-based delivery such as plasmid DNA. Here several Cpf1 orthologs were made as RNPs and edited robustly at multiple genomic loci that were also targetable by SpCas9 in multiple cell types. Editing over 90% in T cells with AsCpf1 and its engineered RR and RVR PAM variants were demonstrated.

Improvement of the Cpf1 RNP complex activity, both at the protein and guide level were demonstrated, which improved efficacy across cell types. Collectively, these findings underscore the promise of RNP delivery for Cpf1 nucleases for genome editing therapeutics.

Results

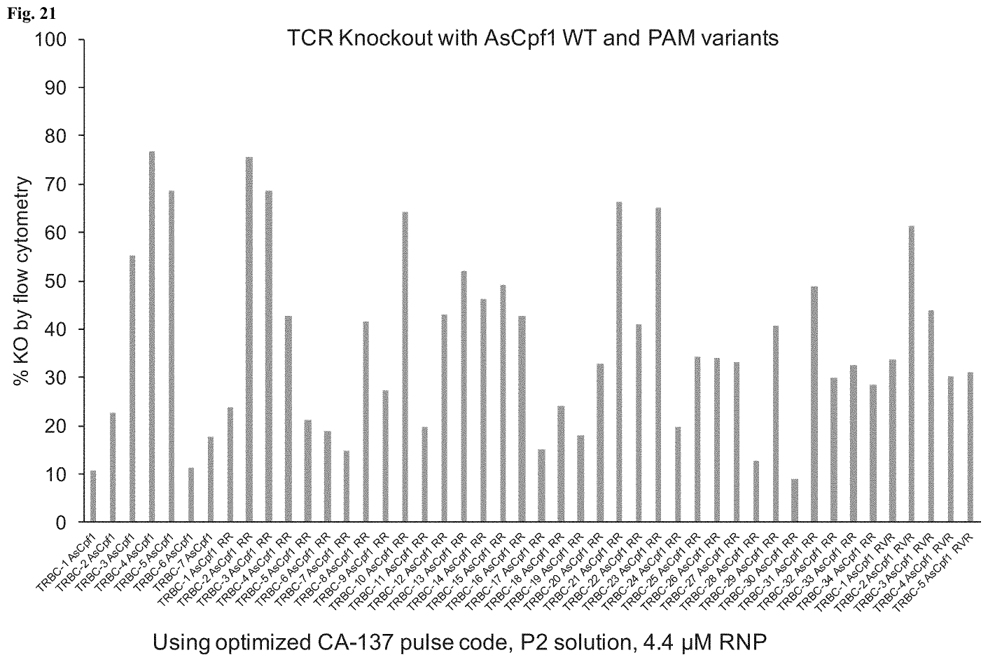

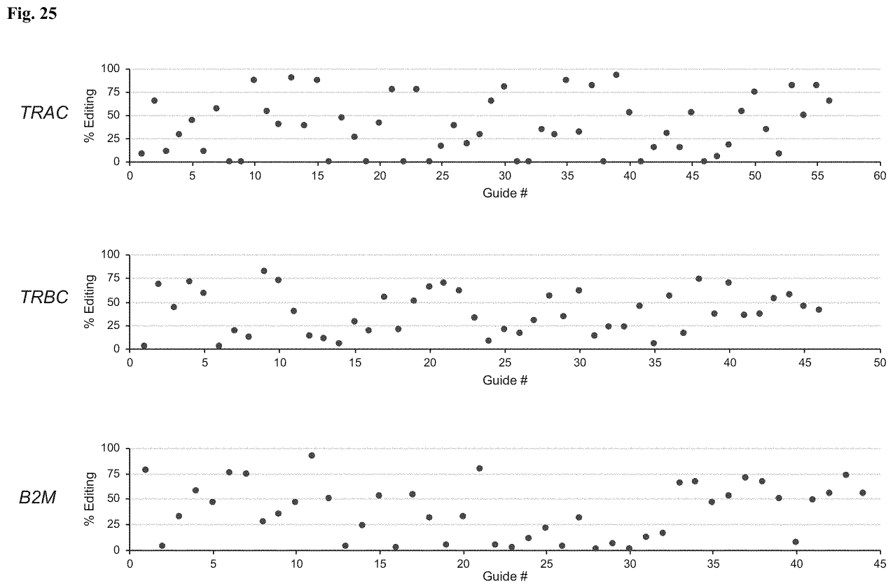

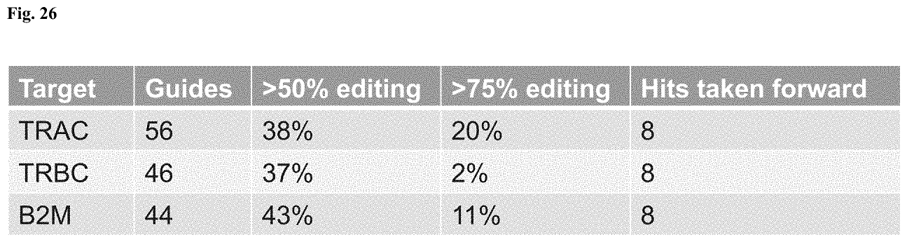

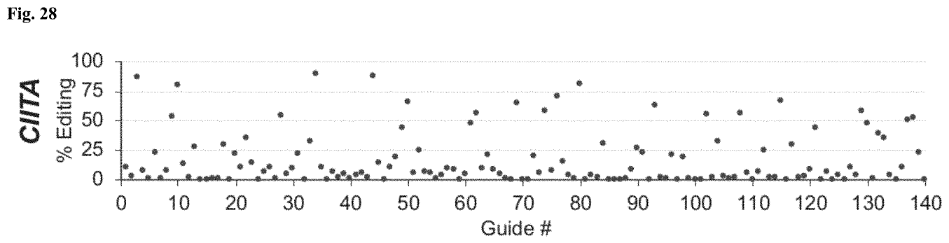

AsCpf1 was selected from several tested Cpf1 orthologs. An AsCpf1 screen in primary T cells yielded several suitable target sites. FIG. 21 and FIG. 25 depict screening of a T cell therapeutic targets with AsCpf1 and its RR and RVR PAM variants at TRBC, TRAC and B2M loci. The sequences of the guide RNAs tested in FIG. 21 are provided in Table 4. For each target, 500,000 T cells were electroporated with 2 μL of 50 μM Cas9 or Cpf1 TRAC guide (2:1 ratio guide to protein) for a final concentration of 4.4 μM using the Amaxa nucleofector (Lonza) with pulse code CA-137 and buffer P2. Percent knockout of protein was measured by flow cytometry. About 30% of gRNAs showed more than 50% editing in the preliminary screen which was on par with generally observed SpCas9 hit rate, showing that Cpf1 can potentially be used for gene editing a patient's T cells at a key therapeutic locus or multiple therapeutic loci. The results outlined in FIG. 21, FIG. 25 and FIG. 28 indicate high editing efficiency for AsCpf1 WT, RR, and RVR in T cells on four allogeneic T cell targets (TRBC, TRAC, B2M and CIITA), which is summarized in FIG. 26. In particular, between 37-43% of the guides give >50% editing and are classified as hits.

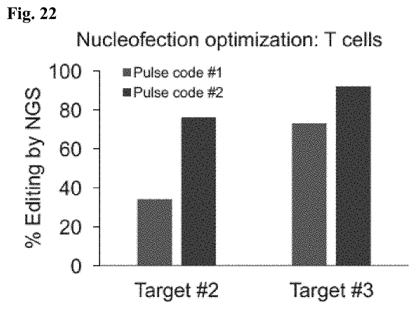

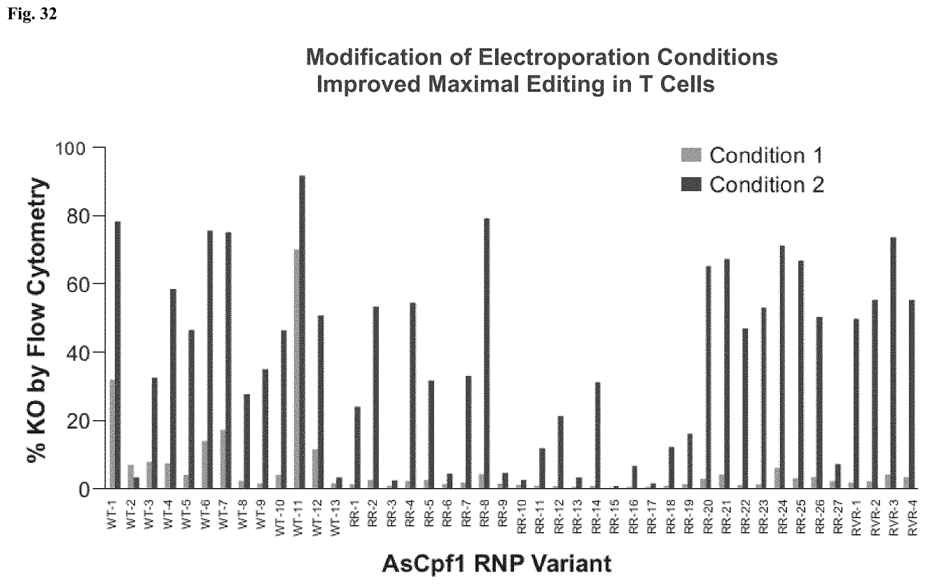

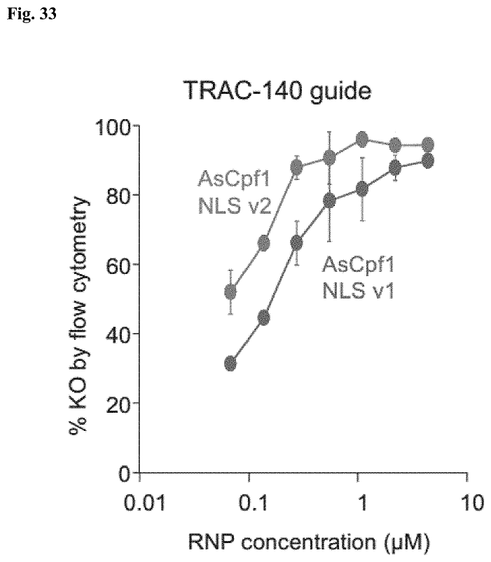

Efficient editing in T cells was achieved by modifying the NLS configuration and electroporation conditions. CAR and TCR engineered T cell therapies have the potential to be transformative additions to the immuno-oncology landscape. As shown in FIG. 32, certain electroporation conditions improved maximal editing in T cells. The guide RNA labeled as RR-25 in FIG. 32 is also referred to herein as “B2M-2,” “B2M-29” and “B2M29-RR” herein. The guide RNA labeled as WT-11 in FIG. 32 is also referred to herein as “B2M-1,” “B2M-12” and “B2M12-WT” herein. Further, changes in the electroporation pulse code also improved maximal editing significantly in T cells at multiple therapeutic target loci as shown in FIG. 22. Target #2 was TRBC and Target #3 was B2M. Pulse code #1 was DS-130 (also referred to herein as “Condition 1”) and Pulse code #2 was CA-137 (also referred to herein as “Condition 2”). For each target, 500,000 T cells were electroporated with 2 μL of 50 μM Cas9 or Cpf1 RNP with a guide targeting TRBC or B2M (2:1 ratio guide to protein) for a final concentration of 4.4 μM for each RNP using the Amaxa nucleofector (Lonza) with pulse code DS-130 and buffer P2 or pulse code CA-137 and buffer P2. Percent knockout of protein was measured by flow cytometry four days later. As shown in FIG. 33, modification of NLS configuration also improved potency in T cells. AspCpf1 NLS v2 (also referred to herein as “His-AsCpf1-sNLS-sNLS”) exhibited better editing efficiency than AspCpf1 NLS v1 (also referred to herein as “His-AsCpf1-sNLS”).

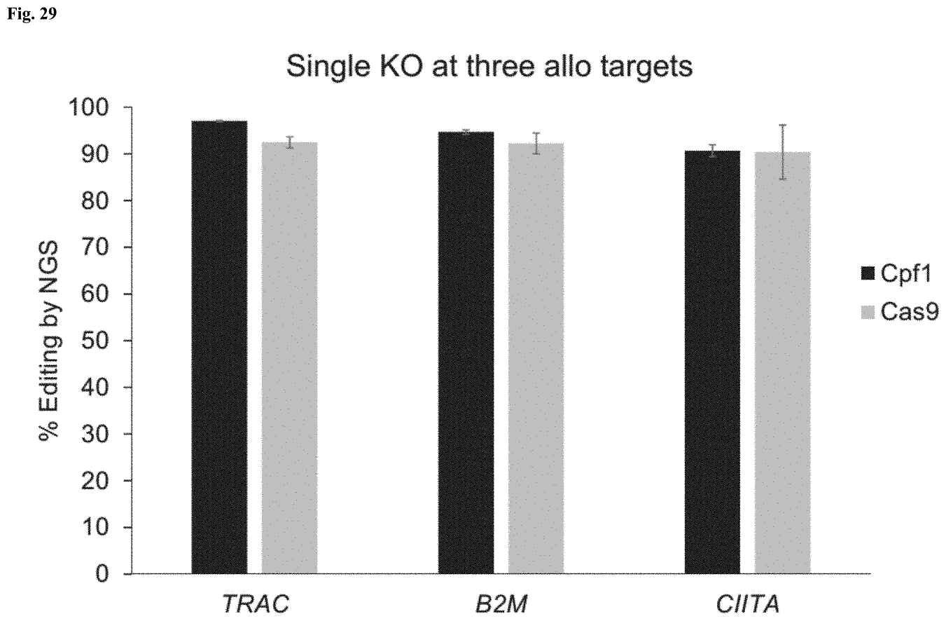

Efficient single and multiple knockout editing were achieved in primary T cells at disease relevant loci with Cpf1 RNPs. FIG. 23A depicts RNP workflow for an ex-vivo cellular therapy. Efficient single knockout at multiple therapeutically relevant T cell loci (TRAC, TRBC and B2M) using AsCpf1 or an engineered PAM variant is shown in FIG. 23B. Comparison was made on single knockout at three T cell targets (TRAC, TRBC and B2M). For each target, 500,000 T cells were electroporated with 2 μL of 50 μM Cas9 or Cpf1 RNP (2:1 ratio guide to protein) for a final concentration of 4.4 μM using the Amaxa nucleofector (Lonza) with pulse code DS-130 and buffer P2. Percent knockout of protein was measured by flow cytometry four days later. TRAC guide was TRAC-140 (also referred to herein as “TRAC-2” and “TRAC-14ORR”) with AsCpf1 RR enzyme. TRBC guide was TRBC-4 with AsCpf1 WT enzyme. B2M guide was B2M-12 with AsCpf1 WT enzyme. FIG. 29 shows the efficiency of a single knockout at multiple therapeutically relevant T cell loci using Cpf1 RNPs as compared to SpCas 9.

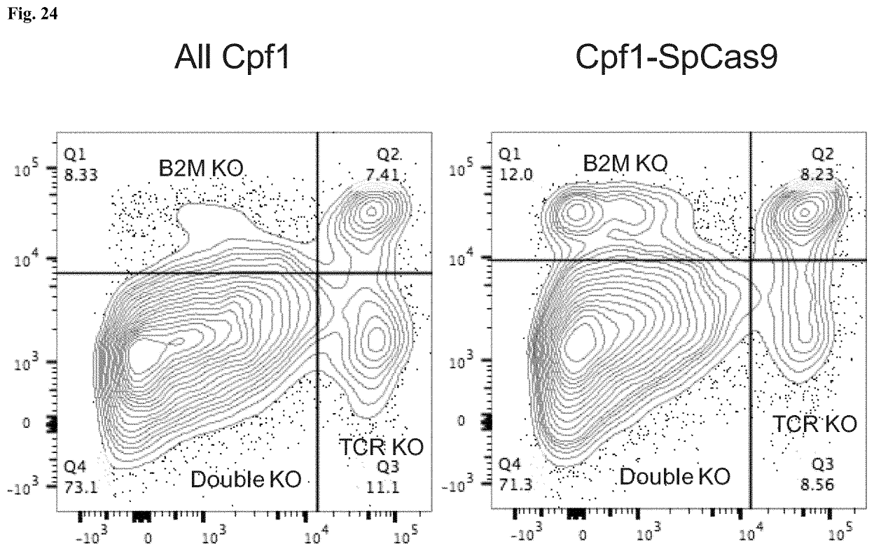

Highly efficient double knockout of two therapeutic targets (TCR and B2M) in T cells treated with Cpf1 RNP was measured by flow cytometry as shown in FIG. 24. FIG. 24 shows the distribution of T cells that had TRAC and B2M effectively knocked down. For each target, 500,000 T cells were electroporated with 1 μL of 100 μM Cas9 or Cpf1 RNP with a guide targeting TRAC along with 1 μL of 100 μM Cas9 or Cpf1 RNP with a guide targeting B2M (2:1 ratio guide to protein) for a final concentration of 4.4 μM for each RNP using the Amaxa nucleofector (Lonza) with pulse code DS-130 and buffer P2. Protein % KO was measured by flow cytometry four days later. TRAC guide was TRAC-140 with AsCpf1 RR enzyme. B2M guide was B2M-12 with AsCpf1 WT enzyme.

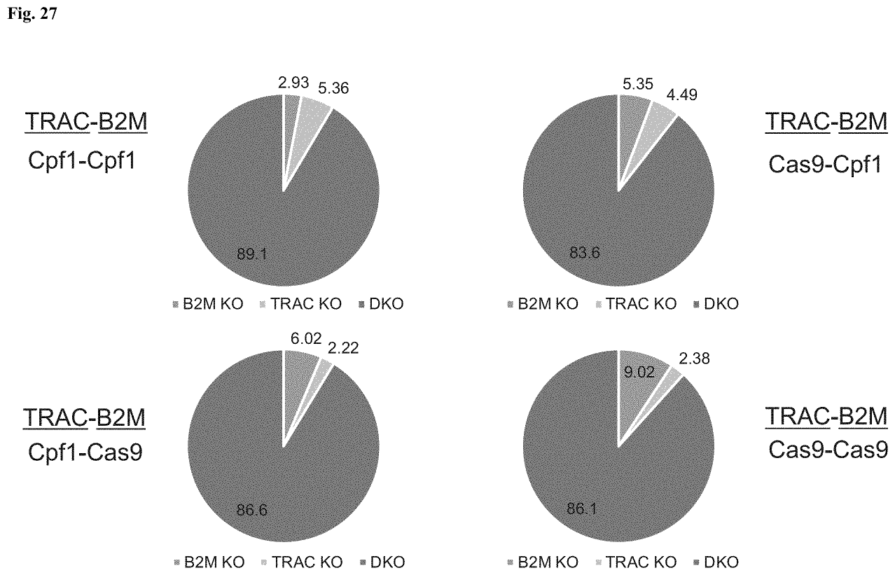

FIG. 27 illustrates the double knockout of two T cell targets, B2M and TRAC, with Cpf1 or Cas9 in human primary T cells. For each target, 500,000 T cells were electroporated with 2 μL of 50 μM Cas9 or Cpf1 protein complexed with either a TRAC or B2M Cas9 or Cpf1 guide (4:1 ratio guide to protein) for a final RNP concentration of 4.4 μM using the Amaxa nucleofector (Lonza) with pulse code CA-137 and buffer P2. Percent knockout of protein was measured by flow cytometry. As shown in FIG. 27, most of the T cells were successfully edited to knock down both B2M and TRAC. These results also show that different nucleases can be used for each T cell target. The Cpf1 TRAC guide used was TRAC-140 and the Cpf1 B2M guide used was B2M-12.

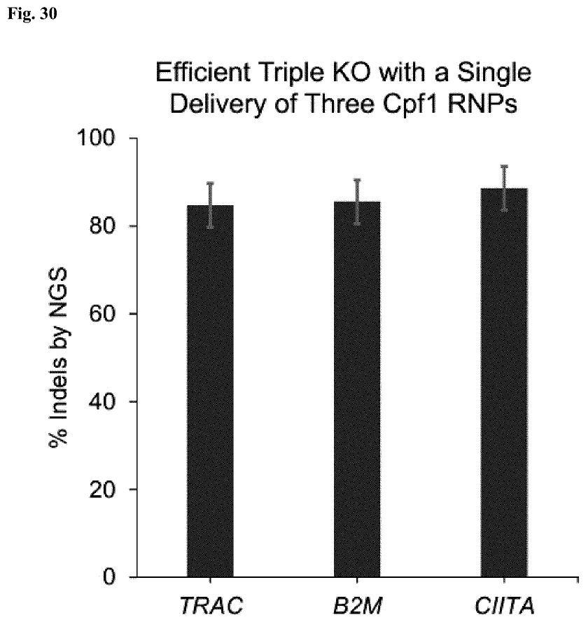

FIG. 30 illustrates the triple knockout of three T cell targets (TRAC, B2M and CIITA) with Cpf1 RNPs in human primary T cells. Percent knockout of protein was measured by flow cytometry. As shown in FIG. 30, efficient editing at all three T cell targets was observed. This experiment was performed with 2.9 μM of RNP with AsCpf1 RR (PRO282) complexed with TRAC guide TRAC-140, 2.9 μM of RNP containing AsCpf1 WT (PRO281) complexed with B2M guide B2M-12, and 2.9 μM of RNP containing AsCpf1 WT (PRO281) complexed with CIITA guide CIITA-34 were delivered together for a total RNP concentration of 8.7 μM. The guide:protein ratio for each RNP was 2:1. RNP was delivered to 500,000 T cells using pulse code CA-137 and buffer P2 on the Lonza system. Editing was assessed by flow cytometry and NGS for TRAC and B2M and by NGS only for CIITA. Similar results were also obtained using TRAC guide TRAC-13, B2M guide B2M-29, and CIITA guides CIITA-45, CIITA-41 and CIITA-10 under the same conditions.

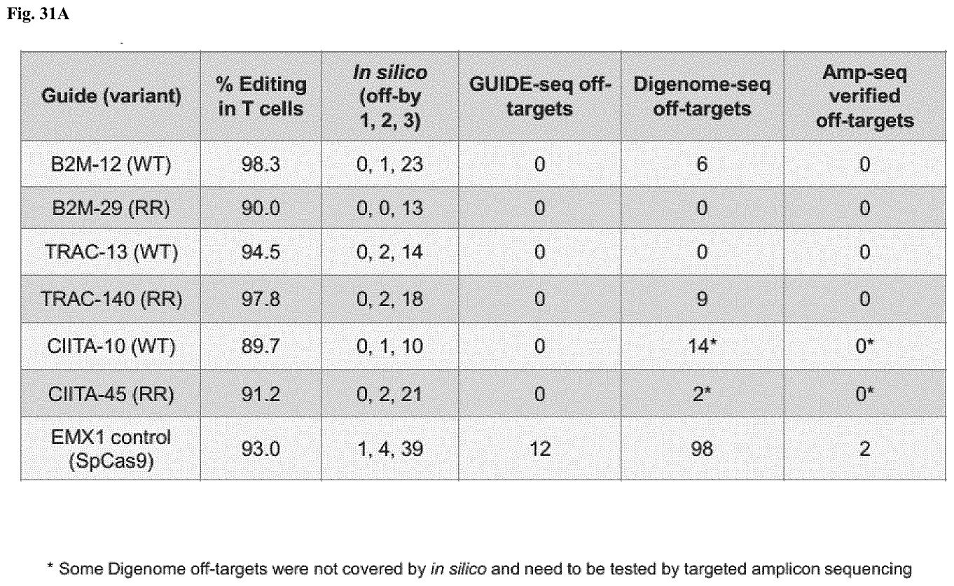

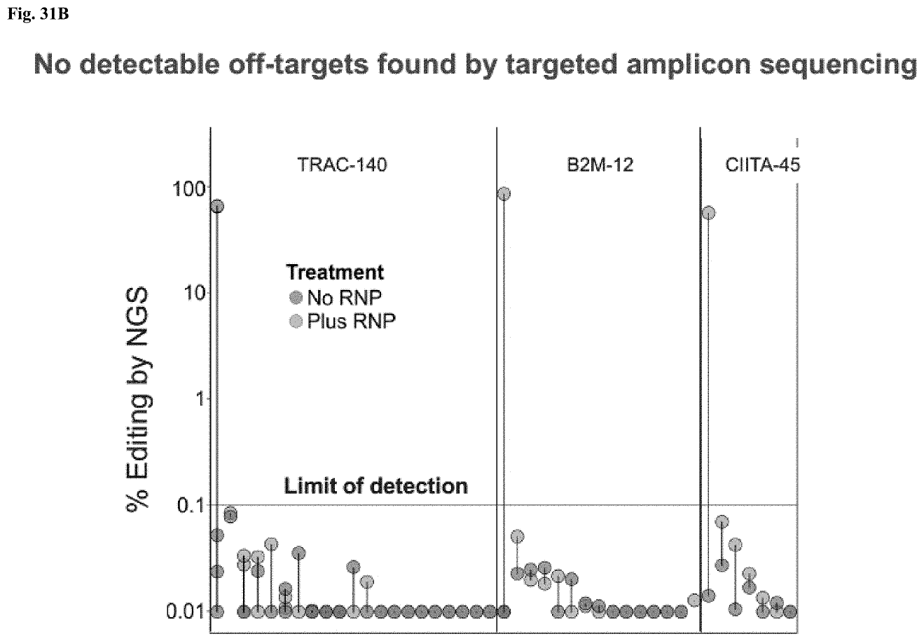

FIG. 31A illustrates the workflow used to identify and verify potential off-targets. FIG. 31B summarizes the specificity of the top Cpf1 candidate guides for three T cell targets, CIITA, TRAC and B2M. As shown in FIG. 31A and FIG. 31B, no detectable off-targets were found by targeted amplicon sequencing of potential off-target sites from in silico, Digenome-seq and GUIDE-seq off-target assays and all the guide RNAs tested resulted in high editing efficiency.

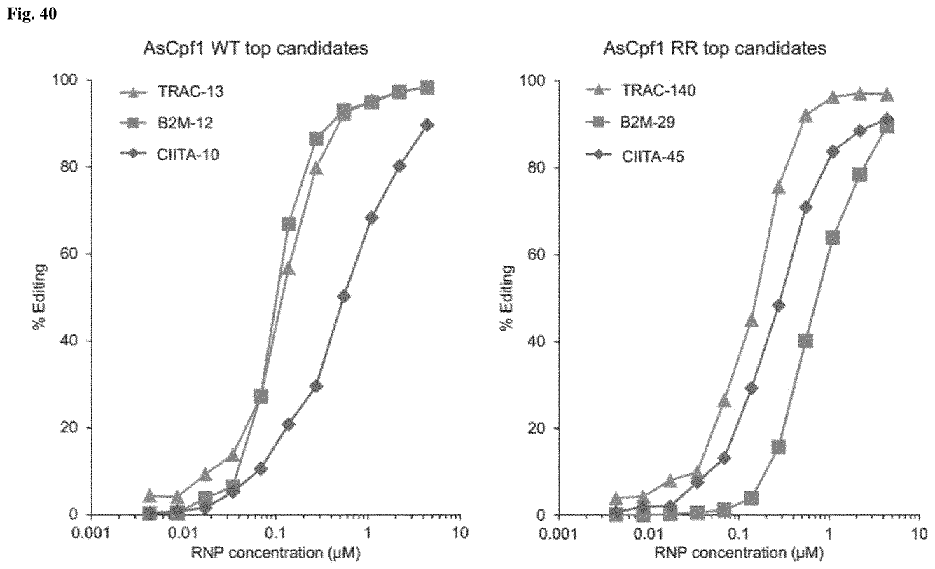

FIG. 40 shows the dose response of the top allogeneic guide RNAs in T cells for WT AsCpf1 and the RR AsCpf1 variant for T cell targets, TRAC, B2M and CIITA. Genomic DNA from cells treated with the highest dose of RNPs were sent for targeted amplicon sequencing to assess indels at each of the guides respective target site. This experiment was performed in T cells using Lonza electroporator and pulse code CA-137.

### Example 6—Phenotypic Analysis of Cpf1-Mediated Knock Out of CIITA

To determine the effect that knocking out CIITA in T cells had on the expression of the major histocompatibility complex class II (MHC II) receptors, cells were transfected with RNPs engineered to target the exons of the CIITA gene. This gene is involved in surface expression of MHC II receptors. Indels in exons result in truncations which inactivate CIITA, and prevent expression of MHC II (HLA DR, DP, DQ) receptors on the T cell surface.

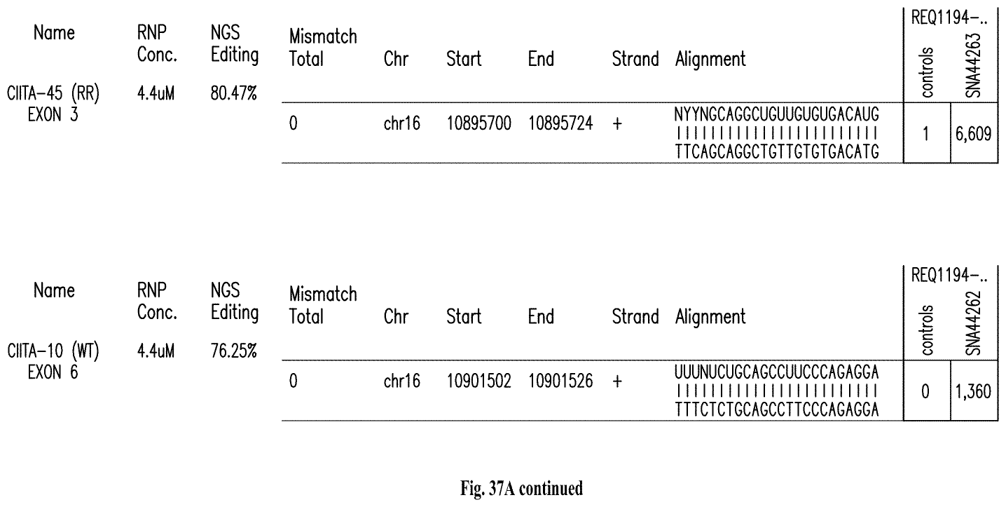

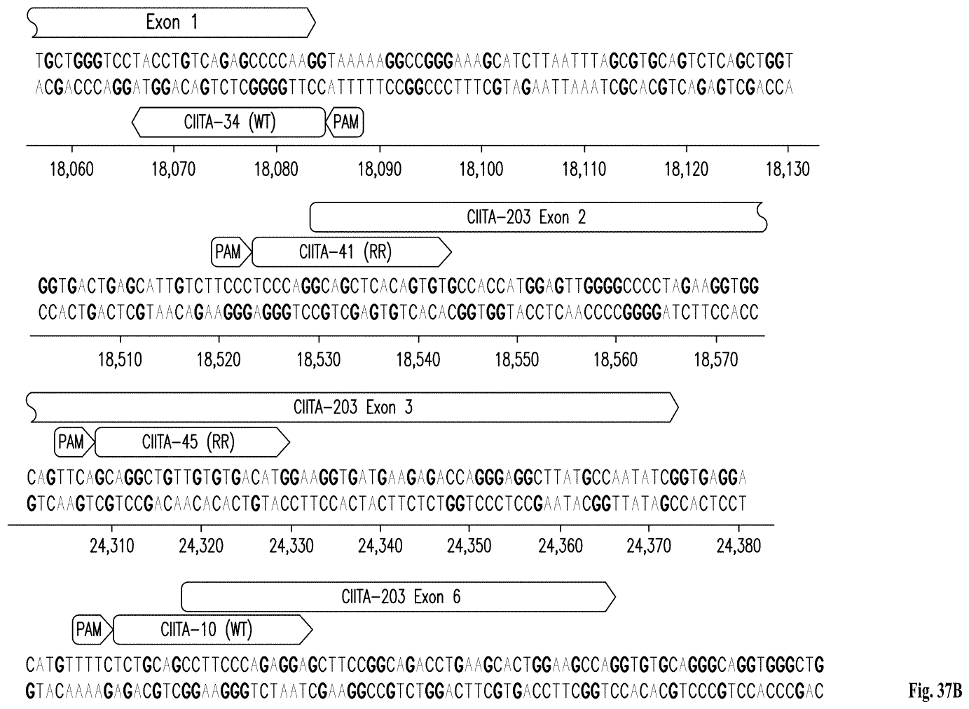

AsCpf1 RNPs were complexed by combining AsCpf1 variants with guide RNA at a 1:2 ratio. The gRNAs used were CIITA-34 (targets Exon 1), CIITA-41 (targets Exon 2), CIITA-45 (targets Exon 3) and CIITA-10 (targets Exon 6) (FIG. 37B). The gRNA CIITA-45 is also referred to herein as “CIITA-45 RR” and “CIITA-2.” The gRNA CIITA-41 is also referred to herein as “CIITA-41 RR.” The gRNA CIITA-34 is also referred to herein as “CIITA-34 WT.” The gRNA CIITA-10 is also referred to herein as “CIITA-1” and “CIITA-10 WT.” The sample was then incubated at room temperature for 30 minutes prior to the tube being submerged in liquid nitrogen and stored at −80° C. until nucleofection. 3 μL RNPs were transferred to each well of a 96 well Lonza nucleofection plate. 500,000 T cells per condition were centrifuged at 1500 rpm for 5 minutes. The pellet was resuspended in 204, of Lonza P2 nucleofection buffer per sample, and then 20 μL of resuspended T cells were added to each well of the Lonza 96 well plate. The cells were promptly nucleofection using the pulse code CA-137. 80 μL of prewarmed (37 C) expansion media was then mixed into each well. The entire volume (3 μL RNPs, 20 μL T cells in P2 buffer, and 80 μL media) was transferred to prewarmed 96 well non-TC treated plates with 100 μL expansion media. The cells were incubated at 37° C. at 5% CO₂ until analysis. On day 4 post nucleofection, a subset of cells was lysed and the genomic DNA was submitted for Illumina sequencing. The remaining cells were expanded to day 6 post nucleofection and then activated using CD3/CD28 beads in stimulation media (High IL-2, IL-7 and IL-15) to stimulate surface expression of MHC II. On day 7, cells were washed off the beads and stained with a monoclonal antibody targeting MHC II (HLA DR, DP, DQ) receptors to phenotypically assess knockout of CIITA. This binding was quantified via flow cytometry.

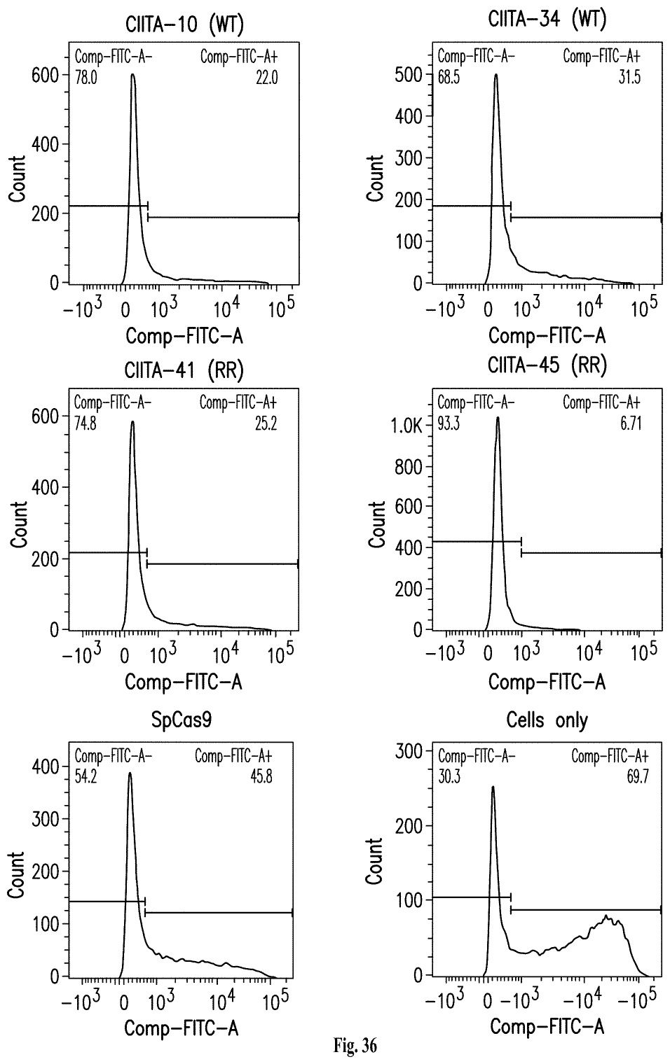

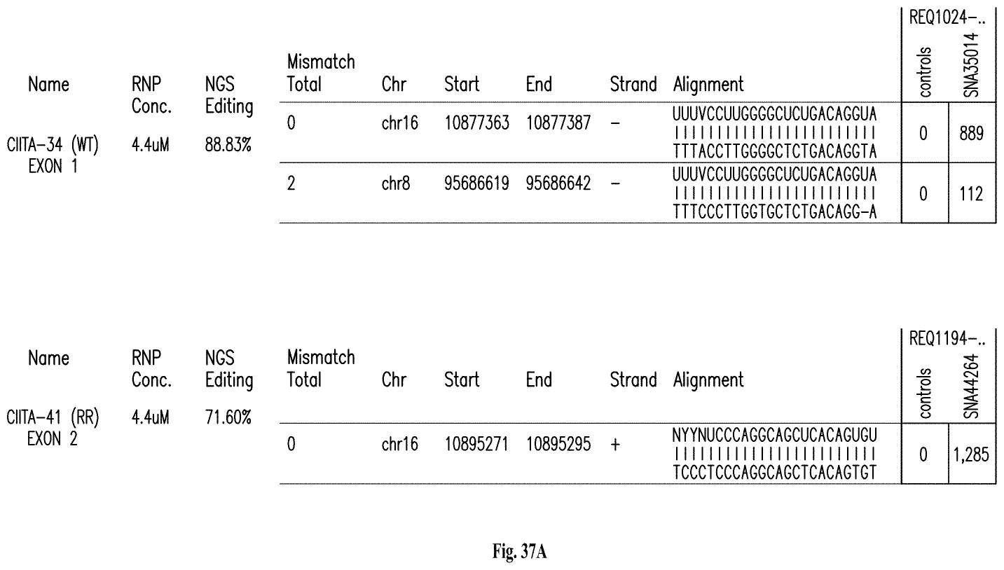

The images provided in FIG. 36 illustrate the detection of the FITC-A fluorophore on the mAb (on the cell surface) by the flow cytometer. The fluorescence intensity directly correlates to the presence or absence of mAb binding to MHC II receptors on the cell surface. High fluorescence indicates high surface expression of MHC II receptors, meanwhile absence of signal indicates successful knockout of these receptors. The X axis indicates increasing (left to right) fluorescence intensity on a logarithmic scale. The Y axis linearly represents incidence of events (cells). The threshold at 10³ was determined as the point which separates the knockout population from the unedited population. Any cells to left of 10³ are classified as knockout cells, and cells to the right of this threshold are considered unedited. As shown in FIG. 36, Cpf1 guide CIITA-45 showed a clear reduction in MHC II positive cells and this is not seen in the untreated cells. T cells were treated with AsCpf1 RR complexed with CIITA-45 using the Lonza system with pulse code CA-137. In addition, the guides had high editing efficiency at the CIITA locus (FIG. 37A).

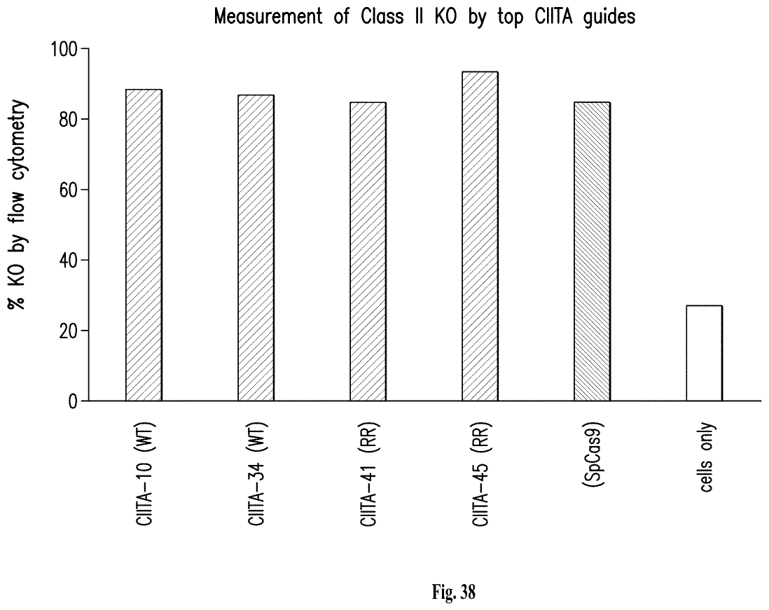

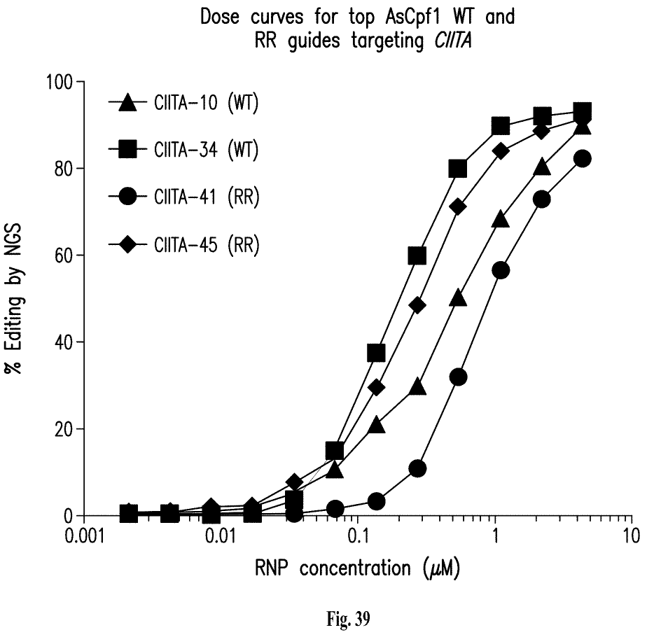

Guides CIITA-41, CIITA-10, and CIITA-34 showed a similar reduction of MHC II as CIITA-45 (FIG. 38). This data validated that each of these four guides not only editing CIITA efficiently but also showed the desired phenotypic effect. A SpCas9 guide known to edit CIITA with high efficiency is shown as a positive control. All Cpf1 or SpCas9 CIITA guides were tested with a 4 μM RNP dose in T cells using the Lonza system with pulse code CA-137. The genomic DNA from cells treated with highest dose of RNPs were sent for targeted amplicon sequencing to assess indels at off target sites. No indel formation was observed above the threshold for detection at predicted off target sites for CIITA-45 and CIITA-10 while CIITA-34 did have one off-target (FIG. 39).

### Example 7—Protospacer Length on Editing Efficiency

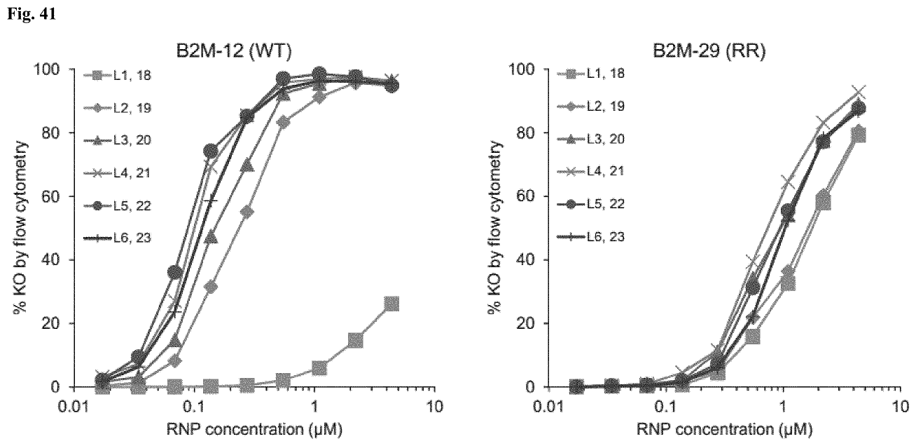

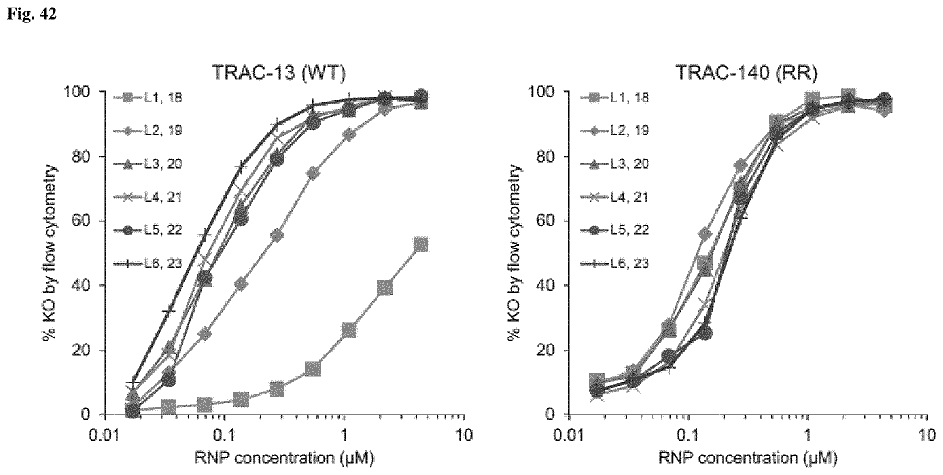

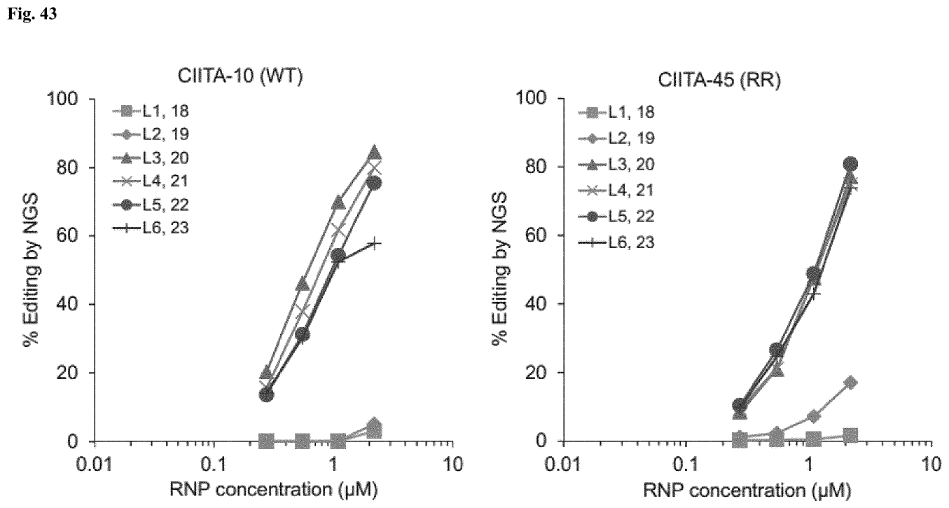

The standard protospacer for a guide RNA is 20 nucleotides long, and this sequence is complementary to the target DNA sequence. By increasing or decreasing the number of nucleotides that are complementary to the target sequence, the binding energy of the guide RNA to its target DNA can be altered and the percentage of indel formed can be altered. Adjusting the length to 18 and 19 reduces indel formation for guides B2M-12, B2M-29, TRAC-13 (also referred to herein as “TRAC-13 WT” and “TRAC-1”), CIITA-10, and CIITA-45 (FIG. 41, FIG. 42 and FIG. 43). Further, as shown in FIG. 41, FIG. 42 and FIG. 43, increasing the length from 20 to 21, 22 or 23 nucleotides has minimal effects on some guides such as TRAC-140 and enhances potency of most others such as TRAC-13, B2M-12, B2M-29, CIITA-10, and CIITA-45. Experiments were performed in T cells in dose response with either AsCpf1 WT or AsCpf1 RR using the Lonza electroporator and pulse code CA-137.

### Example 8—Targeted Integration at the TRAC Locus

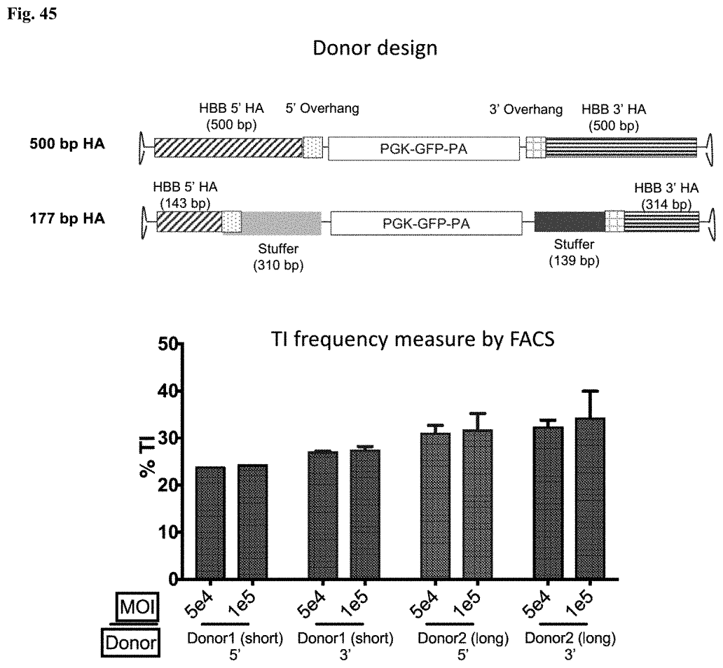

Exemplary DNA donor templates were designed for gRNA targeting the T cell receptor alpha constant (TRAC) locus, as shown in FIG. 45. Each donor contained the same cargo (hPGK-GFP-polyA sequence), but with different homology arm sequences including the 5′ and 3′ overhang regions (Table 14). The homology arm length and arm sequences for each donor is provided in Table Y and Z respectively. Donor1 has a stuffer sequence (Table 16) to keep both donor lengths similar. Targeted integration experiments were conducted in primary CD4+ T cells using AsCpf1RR ribonucleoprotein with the appropriate gRNA and associated AAV donor template at two donor concentrations. Cells were expanded after the experiment until Day 7, when flow cytometry was conducted to check the rate of targeted integration by GFP expression.

The gRNA that was used is TRAC-140: GUGACAAGUCUGUCUGCCUA (RNA sequence); GTGACAAGTCTGTCTGCCTA (DNA sequence).

TABLE 14
Donors for targeted integration at the TRAC locus

| Donor | | | | |
|---|---|---|---|---|
| name | gRNA | HA Length | Stuffer | Cargo |
| Donor1 | TRAC-140 | Short | Yes | PGK + GFP |
| Donor2 | TRAC-140 | Long (500 bp) | No | PGK + GFP |

TABLE 15
Homology Arm (HA) Length in donor templates
for targeted integration at the TRAC

| | 5′ HA Length | 3′ HA Length |
|---|---|---|
| Donor1 | 143 bp + 4 bp overhang | 314 bp + 4 bp overhang |
| Donor2 | 500 bp + 4 bp overhang | 500 bp + 4 bp overhang |

TABLE 16
Homology Arm (HA) Sequences for TRAC donor templates

| | HA Sequences |
|---|---|
| Donor1 | 5′: |
| | ACTCCAGCCTGGGTTGGGGCAAAGAGGGAAATGAGATCATGTCCTAACCCT |
| | GATCCTCTTGTCCCACAGATATCCAGAACCCTGACCCTGCCGTGTACCAGCT |
| | GAGAGACTCTAAATCGAGTGACAAGTCTGTCTGCCTATTC |
| | 3': |
| | ATTCACCGATTTTGATTCTCAAACAAATGTGTCACAAAGTAAGGATTCTGAT |
| | GTGTATATCACAGACAAAACTGTGCTAGACATGAGGTCTATGGACTTCAAG |
| | AGCAACAGTGCTGTGGCCTGGAGCAACAAATCTGACTTTGCATGTGCAAAC |
| | GCCTTCAACAACAGCATTATTCCAGAAGACACCTTCTTCCCCAGCCCAGGTA |
| | AGGGCAGCTTTGGTGCCTTCGCAGGCTGTTTCCTTGCTTCAGGAATGGCCAG |
| | GTTCTGCCCAGAGCTCTGGTCAATGATGTCTAAAACTCCTCTGATTGGTGGT |
| | CTCG |
| Donor2 | 5′: |
| | TGGGGAGACCACTCCAGATTCCAAGATGTACAGTTTGCTTTGCTGGGCCTTT |
| | TTCCCATGCCTGCCTTTACTCTGCCAGAGTTATATTGCTGGGGTTTTGAAGA |
| | AGATCCTATTAAATAAAAGAATAAGCAGTATTATTAAGTAGCCCTGCATTTC |
| | AGGTTTCCTTGAGTGGCAGGCCAGGCCTGGCGTGAACGTTCACTGAAATCA |
| | TGGCCTCTTGGCCAAGATTGATAGCTTGTGCCTGTCCCTGAGTCCCAGTCCA |
| | TCACGAGCAGCTGGTTTCTAAGATGCTATTTCCCGTATAAAGCATGAGACCG |
| | TGACTTGCCAGCCCCACAGAGCCCCGCCCTTGTCCATCACTGGCATCTGGAC |
| | TCCAGCCTGGGTTGGGGCAAAGAGGGAAATGAGATCATGTCCTAACCCTGA |
| | TCCTCTTGTCCCACAGATATCCAGAACCCTGACCCTGCCGTGTACCAGCTGA |
| | GAGACTCTAAATCGAGTGACAAGTCTGTCTGCCT |
| | 3′: |
| | ACCGATTTTGATTCTCAAACAAATGTGTCACAAAGTAAGGATTCTGATGTGT |
| | ATATCACAGACAAAACTGTGCTAGACATGAGGTCTATGGACTTCAAGAGCA |
| | ACAGTGCTGTGGCCTGGAGCAACAAATCTGACTTTGCATGTGCAAACGCCTT |
| | CAACAACAGCATTATTCCAGAAGACACCTTCTTCCCCAGCCCAGGTAAGGG |
| | CAGCTTTGGTGCCTTCGCAGGCTGTTTCCTTGCTTCAGGAATGGCCAGGTTC |
| | TGCCCAGAGCTCTGGTCAATGATGTCTAAAACTCCTCTGATTGGTGGTCTCG |
| | GCCTTATCCATTGCCACCAAAACCCTCTTTTTACTAAGAAACAGTGAGCCTT |
| | GTTCTGGCAGTCCAGAGAATGACACGGGAAAAAAGCAGATGAAGAGAAGG |
| | TGGCAGGAGAGGGCACGTGGCCCAGCCTCAGTCTCTCCAACTGAGTTCCTG |
| | CCTGCCTGCCTTTGCTCAGACTGTTTGCCCCTTACTG |
| Stuffer for | 5′: |
| Donor1 | TACTCTTAATTCATTACATATTGTGCGGTCGAATTCAGGGAGCCGATAATGC |
| | GGTTACAATAATTCCTATACTTAAATATACAAAGATTTAAAATTTCAAAAAA |
| | TGGTTACCAGCATCGTTAGTGCGTATACATCAAGAGGCACGTGCCCCGGAG |
| | ACAGCAAGTAAGCTCTTTAAACATGCTTTGACATACGATTTTTAATAAAACA |
| | TGAGCATTTGAATAAAAACGACTTCCTCATACTGTAAACATCACGCATGCAC |
| | ATTAGACAATAATCCAGTAACGAAACGGCTTCAGTCGTAATCGCCCATATA |
| | 3′: |
| | GCATATTACGGAATAATCCTATCGTTATCAGATCTCCCCTGTCATATCACAA |
| | CATGTTTCGATGTTCCAAAACCGGGAACATTTTGGATCGGTTAAATGATTGT |
| | ACATCATTTGTTGCAGACCTTAGGAACATCCATCA |

Targeted integration efficiency at the TRAC locus using higher AAV donor concentration is shown in Table 17.

TABLE 17
Targeted integration frequency

| | Flow Cytometry (GFP) |
|---|---|
| Donor1 | 26.5% |
| Donor2 | 33.5% |

As shown in Table 17, donor templates containing long homology arms (500 bp) had slightly higher levels of targeted integration than donors containing shorter homology arms.

### Example 9: Screen of Cpf1 gRNAs Targeting the HBG Promoter Region

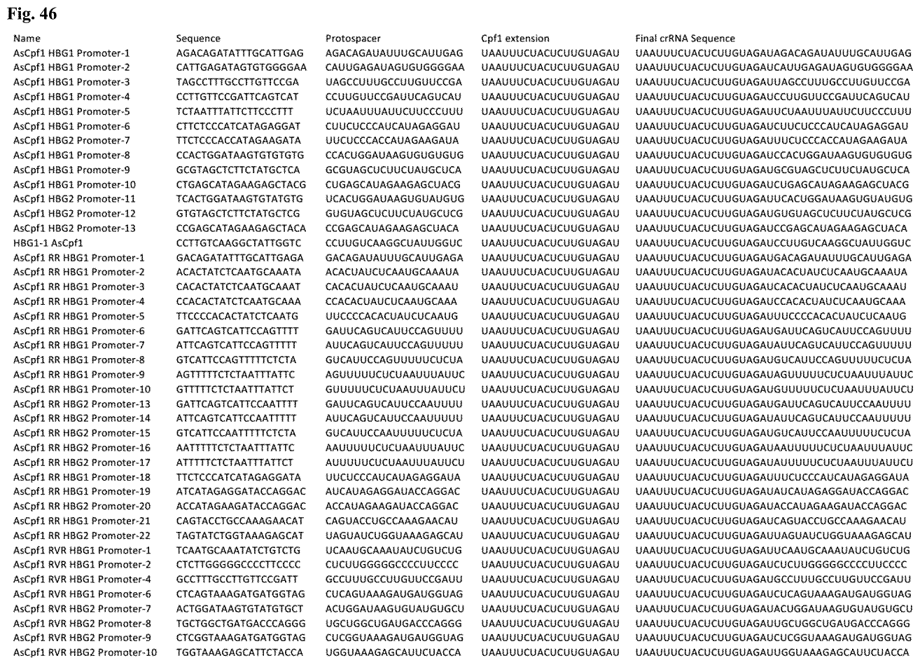

To identify other AsCpf1 gRNA that could be used as a component of a single RNP or in combination with a “booster element” to increase editing of the HBG promoter region in CD34+ cells and induce fetal globin expression in the erythroid progeny of modified cells, His-AsCpf1-NLS-NLS (“AsCpf1”); AsCpf1 S542R/K607R (“AsCpf1 RR); or AsCpf1 S542R/K548V/N552R (“AsCpf1 RVR”) gRNA sequences targeting several domains of the HBG promoter (Table 18) were designed (listed in Table 19 and FIG. 46). AsCpf1 RR and AsCpf1 RVR are engineered AsCpf1 variants which recognize TYCV/ACCC/CCCC and TATV/RATR PAMs, respectively (Gao 2017).

TABLE 18
Subdomains of the HBG genomic region

| Genomic | | |
|---|---|---|
| Coordinate of | | |
| HBG* | Nucleotides | Name of Region |
| Chr 11 | TCCTAAAGCT TGGAACACTT | Region 1: Downstream of |
| (NC_000011.10): | TCCCTTCCTT AAGAACCATC | HBG1 |
| 5,247,883- | CTTGCTACTC AGCTGCAATC | |
| 5,248,186 | AATCCAGCCC CCAGGTCTTC | |
| | ACTGAACCTT TTCCCATCTC | |
| | TTCCAAAACA TCTGTTTCTG | |
| | AGAAGTCCTG TCCTATAGAG | |
| | GTCTTTCTTC CCACCGGATT | |
| | TCTCCTACAC CATTTACTCC | |
| | CACTTGCAGA ACTCCCGTGT | |
| | ACAAGTGTCT TTACTGCTTT | |
| | TATTTGCTCA TCAAAATGCA | |
| | CATCTCATAT AAAAATAAAT | |
| | GAGGAGCATG CACACACCAC | |
| | AAACACAAAC AGGCATGCAG | |
| | AAAT | |
| Chr 11 | ATAAAGATGA ACCCATAGTG | Region 2: HBG1 Intron 2 - A |
| (NC_000011.10): | AGCTGAGAGC TCCAGCCTGG | |
| 5,248,509- | CCTCCAGATA ACTACACACC | |
| 5,249,173 | AAGCTTCCAC CCAGAATCAA | |
| | GCCTATGTTA ACTTCCCTCA | |
| | AAGCCTGAGA TTTTGCCTTC | |
| | CCATTAAATG CAGGTAGTTG | |
| | TTCCCCTTCA AGCACTAGTC | |
| | ACTGGCCATA ATTTAAATCT | |
| | TGCTATCTTC TTGCCACCAT | |
| | GAACCCTGTA TGTTGTAGGC | |
| | TGAAGACGTT AAAAGAAACA | |
| | CACGCTGACA CACACACACA | |
| | CACGCGCGCG CGCACACACA | |
| | CACACACACA CAGAGCTGAC | |
| | TTTCAAAATC TACTCCAGCC | |
| | CAAATGTTTC AATTGTTCCT | |
| | CACCCCTGGA CATACTTTGC | |
| | CCCCATCTGG AATTAAAGGA | |
| | TATAAGTTTG TAATGAAGCA | |
| | TTAGCAGCAT TTTATATGTG | |
| | TCCAGCTGAT ATAGGAATAG | |
| | CCTTAGCAAT GTATGTTTGG | |
| | CCACCAAAGT TCCCCACTTT | |
| | GACTGAGCCA ATATATGCCT | |
| | TCTGCCTGCA TCTTTTTAAC | |
| | GACCATACTT GTCCTGCCTC | |
| | CAGATAGATG TTTTAAAACA | |
| | ACAAAAATGA GGGAAAGATG | |
| | AAAGTTCTTT CTACTGGAAT | |
| | CTAATAAAGA AAAGTCATTT | |
| | TCCTCATTTC CACCTCTCTT | |
| | TTCTCAAAGT CAAAATTGTC | |
| | CATCT | |
| Chr 11 | CCCTAAAACA TTACCACTGG | Region 3: HBG1 Intron 2- B |
| (NC_000011.10): | GTCTCAGCCC AGTTAGTCCT | |
| 5,249,198- | CTGCAGTTTC TTCACCCCCA | |
| 5,249,362 | ACCCCAGTAT CTTCAAACAG | |
| | CTCACACCCT GCTGTGCTCA | |
| | GATCAATACT CCGTTGTCTA | |
| | AGTTGCCTCG AGACTAAAGG | |
| | CAACAGGGCT GAAACATCTC | |
| | CTGGA | |
| Chr 11 | CTGTGAGATT GACAAGAACA | Region 4: HBG1 Intron 1 |
| (NC_000011.10): | GTTTGACAGT CAGAAGGTGC | |
| 5,249,591- | CACAAATCCT GAGAAGCGAC | |
| 5,249,712 | CTGGACTTTT GCCAGGCACA | |
| | GGGTCCTTCC TTCCCTCCCT | |
| | TGTCCTGGTC ACCAGAGCCT AC | |
| Chr 11 | GCCGCCGGCC CCTGGCCTCA | Region 5: HBG1 −60 nt region |
| (NC_000011.10): | CTGG | from Transcription Start Site |
| 5,249,904- | | (TSS) |
| 5,249,927 | | |
| Chr 11 | CCTTGTCAAG GCTATTGGTC | Region 6: HBG1 −110 nt region |
| (NC_000011.10): | AAGGCAAGGC TGG | from TSS |
| 5,249,955- | | |
| 5,249,987 | | |
| Chr 11 | TGAGATAGTG TGGGGAAGGG | Region 7: HBG1 −200 nt region |
| (NC_000011.10): | GCCCCC AAGAGGATAC | from TSS |
| 5,250,040- | | |
| 5,250,075 | | |
| Chr 11 | TATAGCCTTT GCCTTGTTCC | Region 8: HBG1 −250 nt region |
| (NC_000011.10): | GATTCAGTCA TTCCAGTTTT T | from TSS |
| 5,250,089- | | |
| 5,250,129 | | |
| Chr 11 | TCTTCCCTTT AGCTAGTTTC | Region 9: HBG1 −333 nt region |
| (NC_000011.10): | CTTCTCCCAT CATAGAGGAT | from TSS |
| 5,250,141- | ACCAGGACTT CTTTTGTCAG | |
| 5,250,254 | CCGTTTTTTA CCTTCTTGTC | |
| | TCTAGCTCCA GTGAGGCCTG | |
| | TAGTTTAAAG CTAA | |
| Chr 11 | CCACAGTTTC AGCGCAGTAA | Region 10: HBG1 −650 nt |
| (NC_000011.10): | TAGATTAGTG TTACATAATA | region from TSS |
| 5,250,464- | TAAGACCTAA TGCTTACCTC | |
| 5,250,549 | AATATCTACT TATCCGTACC | |
| | TATTTG | |
| Chr 11 | TATTCAGGTA TGTATGTATA | Region 11: HBG1 −800 nt |
| (NC_000011.10): | CACCAGATGA TGTGTATTTA | region from TSS |
| 5,250,594- | CCACTGGATA AGTGTGTGTG | |
| 5,250,735 | CTGGCTGATG ACCCAGGGTT | |
| | TTGGCGTAGC TCTTCTATGC | |
| | TCAGTAAAGA TGATGGTAGA | |
| | ATGTTCTTTG GCAGGTACTG TG | |
| Chr 11 | CAATAAAGAT GAACCCATAG | Region 12: HBG2 Intron 2 - A |
| (NC_000011.10): | TGAGCTGAGA GCTCCAGCCT | |
| 5,253,425- | GGCCTCCAGA TAACTACACA | |
| 5,254,121 | CCAAGCTTCC ACCCAGAATC | |
| | AAGCCTATGT TAACTTCCCT | |
| | CAAAGCCTGA GATTTTGCTT | |
| | TCCCATTAAA TGCAGGTAGT | |
| | TGTTCTTCTT GCAGCACTAG | |
| | TCACTGGCCA TAATTTAAAT | |
| | CTTGTTATCT TCTTGCCACC | |
| | ATGAACCCTG TATGCTGTAG | |
| | GCTGAAAACG TTAAAAGAAA | |
| | CACACGCTCT CACACACACA | |
| | CAAACACACG CGCGCACACA | |
| | CACACACACA CACACAGAGC | |
| | TGACTTTCAA AATCTACTCC | |
| | AGCCCAAATG TTTCAATTGT | |
| | TCCTCACCCC TGGACATACT | |
| | TTGCCCCCAT CTGGAATTAA | |
| | AGGATATAAG TTTGTAATGA | |
| | AGCATTAGCA GCATTTTATA | |
| | TGTGTCCAGC TGATATAGGA | |
| | ATAGCCTTAG CAATGTATGT | |
| | TTGGCCACCA AAGTTCCCCA | |
| | CTTTGACTGA GCCAATATAT | |
| | GCCTTCTGCC TGCATCTTTT | |
| | TAATGACCAT ACTTGTCCTG | |
| | CCTCCAGATA GATGTTTTAA | |
| | AACGAATAAC AAAAATAGGG | |
| | GAAAGGTGAA AGTTCTTTCT | |
| | ACCGAAATCT AATAAAGAAA | |
| | AGTCATTTTC CTCATTTCCA | |
| | CCTCTCTTTT CTCAAAGTCA | |
| | AAGTTGTCCA TCTAGATTTT | |
| | CAGAGGCACT CCTTAGG | |
| Chr 11 | CCCTAAAACA TTGCCACTGG | Region 13: HBG2 Intron 2 - B |
| (NC_000011.10): | GTCTCAGCCC AGTTAGTCCT | |
| 5,254,122- | CTGCAGTTTC TTCACTCCCA | |
| 5,254,306 | ACCCCAGTAT CTTCAAACAG | |
| | CTCACACCCT GCTGTGCTCA | |
| | GATCAATACT CAGTTGTCTA | |
| | AGTTGCCTCG AGACTAAAGG | |
| | CAACAGTGCT GAAACATCTC | |
| | CTGGACTCAC CTTGAAGTTC | |
| | TCAGG | |
| Chr 11 | AGCCTGTGAG ATTGACAAGA | Region 14: HBG2 Intron 1 |
| (NC_000011.10): | ACAGTTTGAC AGTCAGAAGG | |
| 5,254,511- | TGCCACAAAT CCTGAGAAGC | |
| 5,254,648 | GACCTGGACT TTTGCCAGGC | |
| | ACAGGGTCCT TCCTTCCCTC | |
| | CCTTGTCCTG GTCACCAGAG | |
| | CCTACCTTCC CAGGGTT | |
| Chr 11 | CCGCCGGCCC CTGGCCTCAC | Region 15: HBG2 −60 nt region |
| (NC_000011.10): | TGGATACTCT AAGACTAT | from TSS |
| 5,254,829- | | |
| 5,254,866 | | |
| Chr 11 | CCTTGTCAAG GCTATTGGTC | Region 16: HBG2 −110 nt |
| (NC_000011.10): | AAGGCAAGGC T | region from TSS |
| 5,254,879- | | |
| 5,254,909 | | |
| Chr 11 | CAGGGACCGT TTCAGACAGA | Region 17: HBG2 −200 nt |
| (NC_000011.10): | TATTTGCATT GAGATAGTGT | region from TSS |
| 5,254,935 | GGGGAAGGGG CCCCCAAGAG | |
| 5,255,009 | GATACTGCTG CTTAA | |
| Chr 11 | TTGCCTTGTT CCGATTCAGT | Region 18: HBG2 −250 nt |
| (NC_000011.10): | CATTCCAAT | region from TSS |
| 5,255,025- | | |
| 5,255,053 | | |
| Chr 11 | TTTAGCTAGT TTTCTTCTCC | Region 19: HBG2 −330 nt |
| (NC_000011.10): | CACCATAGAA GATACCAGGA | region from TSS |
| 5,255,076- | CTTCTTTTGT CAGCCGTTTT | |
| 5,255,179 | TCACCTTCTT GTCTGTAGCT | |
| | CCAGTGAGGC CTGTAGTTTA | |
| | AAGT | |
| Chr 11 | GGACACGTCT TAGTCTCATT | Region 20: HBG2 −500 nt |
| (NC_000011.10): | TAGTAAGCAT TGGTTTCC | region from TSS |
| 5,255,255- | | |
| 5,255,292 | | |
| Chr 11 | TTTTTTATAT TCAGGTATGT | Region 21: HBG2 −800 nt |
| (NC_000011.10): | ATGTAGGCAC CCGATGATGT | region from TSS |
| 5,255,518- | GTATTTATCA CTGGATAAGT | |
| 5,255,641 | GTATGTGCTG GCTGATGACC | |
| | CAGGGTTTTG GTGTAGCTCT | |
| | TCTATGCTCG GTAAAGATGA | |
| | TGGT | |

*NCBI Reference Sequence NC_000011, the coordinates are reported using the One-based coordinate system, “Homo sapiens chromosome 11, GRCh38.p12 Primary Assembly,” (Version NC_000011.10).

TABLE 19
Cpf1 guide RNAs

| | gRNA | gRNA | | | |
|---|---|---|---|---|---|
| | Targeting | Targeting | | | |
| | domain | domain | Genomic | | |
| | sequence | sequence | coordinates of | % | |
| gRNA ID* | (RNA) | (DNA) | HbG** | Editing | Strand |
| AsCpf1 HBG1 | AGACAGAUA | AGACAGATA | Chr11:5250024:5250043 | 5.43 | + |
| Promoter-1 | UUUGCAUUG | TTTGCATTG | | | |
| | AG | AG | | | |
| AsCpf1 HBG1 | CAUUGAGAU | CATTGAGAT | Chr11:5250037:5250056 | 8.30 | + |
| Promoter-2 | AGUGUGGGG | AGTGTGGGG | | | |
| | AA | AA | | | |
| AsCpf1 HBG1 | UAGCCUUUG | TAGCCTTTG | Chr11:5250091:5250110 | 0.23 | + |
| Promoter-3 | CCUUGUUCC | CCTTGTTCC | | | |
| | GA | GA | | | |
| AsCpf1 HBG1 | CCUUGUUCC | CCTTGTTCC | Chr11:5250100:5250119 | 1.15 | + |
| Promoter-4 | GAUUCAGUC | GATTCAGTC | | | |
| | AU | AT | | | |
| AsCpf1 HBG1 | UCUAAUUUA | TCTAATTTA | Chr11:5250131:5250150 | 0.16 | + |
| Promoter-5 | UUCUUCCCU | TTCTTCCCTT | | | |
| | UU | T | | | |
| AsCpf1 HBG1 | CUUCUCCCA | CTTCTCCCA | Chr11:5250161:5250180 | 12.73 | + |
| Promoter-6 | UCAUAGAGG | TCATAGAGG | | | |
| | AU | AT | | | |
| AsCpf1 HBG2 | UUCUCCCAC | TTCTCCCAC | Chr11:5255090:5255109 | 8.11 | + |
| Promoter-7 | CAUAGAAGA | CATAGAAGA | | | |
| | UA | TA | | | |
| AsCpf1 HBG1 | CCACUGGAU | CCACTGGAT | Chr11:5250634:5250653 | 13.33 | + |
| Promoter-8 | AAGUGUGUG | AAGTGTGTG | | | |
| | UG | TG | | | |
| AsCpf1 HBG1 | GCGUAGCUC | GCGTAGCTC | Chr11:5250677:5250696 | 13.48 | + |
| Promoter-9 | UUCUAUGCU | TTCTATGCT | | | |
| | CA | CA | | | |
| AsCpf1 HBG1 | CUGAGCAUA | CTGAGCATA | Chr11:5250678:5250697 | 10.73 | − |
| Promoter-10 | GAAGAGCUA | GAAGAGCTA | | | |
| | CG | CG | | | |
| AsCpf1 HBG2 | UCACUGGAU | TCACTGGAT | Chr11:5255565:5255584 | 0.43 | + |
| Promoter-11 | AAGUGUAUG | AAGTGTATG | | | |
| | UG | TG | | | |
| AsCpf1 HBG2 | GUGUAGCUC | GTGTAGCTC | Chr11:5255608:5255627 | 5.78 | + |
| Promoter-12 | UUCUAUGCU | TTCTATGCT | | | |
| | CG | CG | | | |
| AsCpf1 HBG2 | CCGAGCAUA | CCGAGCATA | Chr11:5255609:5255628 | 3.24 | − |
| Promoter-13 | GAAGAGCUA | GAAGAGCTA | | | |
| | CA | CA | | | |
| HBG1-1 | CCUUGUCAA | CCTTGTCAA | Chr11:5249955:5249974 | 17.96 | + |
| AsCpf1 | GGCUAUUGG | GGCTATTGG | | | |
| | UC | TC | | | |
| AsCpf1 RR | GACAGAUAU | GACAGATAT | Chr11:5250025:5250044 | 8.48 | + |
| HBG1 | UUGCAUUGA | TTGCATTGA | | | |
| Promoter-1 | GA | GA | | | |
| AsCpf1 RR | ACACUAUCU | ACACTATCT | Chr11:5250031:5250050 | 0.09 | − |
| HBG1 | CAAUGCAAA | CAATGCAAA | | | |
| Promoter-2 | UA | TA | | | |
| AsCpf1 RR | CACACUAUC | CACACTATC | Chr11:5250032:5250051 | 2.10 | − |
| HBG1 | UCAAUGCAA | TCAATGCAA | | | |
| Promoter-3 | AU | AT | | | |
| AsCpf1 RR | CCACACUAU | CCACACTAT | Chr11:5250033:5250052 | 2.52 | − |
| HBG1 | CUCAAUGCA | CTCAATGCA | | | |
| Promoter-4 | AA | AA | | | |
| AsCpf1 RR | UUCCCCACA | TTCCCCACA | Chr11:5250037:5250056 | 0.05 | − |
| HBG1 | CUAUCUCAA | CTATCTCAA | | | |
| Promoter-5 | UG | TG | | | |
| AsCpf1 RR | GAUUCAGUC | GATTCAGTC | Chr11:5250109:5250128 | 0.77 | + |
| HBG1 | AUUCCAGUU | ATTCCAGTT | | | |
| Promoter-6 | UU | TT | | | |
| AsCpf1 RR | AUUCAGUCA | ATTCAGTCA | Chr11:5250110:5250129 | 0.24 | + |
| HBG1 | UUCCAGUUU | TTCCAGTTT | | | |
| Promoter-7 | UU | TT | | | |
| AsCpf1 RR | GUCAUUCCA | GTCATTCCA | Chr11:5250115:5250134 | 1.00 | + |
| HBG1 | GUUUUUCUC | GTTTTTCTCT | | | |
| Promoter-8 | UA | A | | | |
| AsCpf1 RR | AGUUUUUCU | AGTTTTTCT | Chr11:5250123:5250142 | 0.15 | + |
| HBG1 | CUAAUUUAU | CTAATTTAT | | | |
| Promoter-9 | UC | TC | | | |
| AsCpf1 RR | GUUUUUCUC | GTTTTTCTCT | Chr11:5250124:5250143 | 0.15 | + |
| HBG1 | UAAUUUAUU | AATTTATTC | | | |
| Promoter-10 | CU | T | | | |
| AsCpf1 RR | GAUUCAGUC | CAAGAGGAT | Chr11:5254989:5255008 | *** | + |
| HBG2 | AUUCCAAUU | ACTGCTGCT | | | |
| Promoter-11 | UU | TA | | | |
| AsCpf1 RR | AUUCAGUCA | AAGAGGAT | Chr11:5254990:5255009 | *** | + |
| HBG2 | UUCCAAUUU | ACTGCTGCT | | | |
| Promoter-12 | UU | TAA | | | |
| AsCpf1 RR | GUCAUUCCA | GATTCAGTC | Chr11:5255037:5255056 | 0.25 | + |
| HBG2 | AUUUUUCUC | ATTCCAATT | | | |
| Promoter-13 | UA | TT | | | |
| AsCpf1 RR | AAUUUUUCU | ATTCAGTCA | Chr11:5255038:5255057 | 0.13 | + |
| HBG2 | CUAAUUUAU | TTCCAATTT | | | |
| Promoter-14 | UC | TT | | | |
| AsCpf1 RR | AUUUUUCUC | GTCATTCCA | Chr11:5255043:5255062 | 0.32 | + |
| HBG2 | UAAUUUAUU | ATTTTTCTCT | | | |
| Promoter-15 | CU | A | | | |
| AsCpf1 RR | UUCUCCCAU | AATTTTTCT | Chr11:5255051:5255070 | 0.14 | + |
| HBG2 | CAUAGAGGA | CTAATTTAT | | | |
| Promoter-16 | UA | TC | | | |
| AsCpf1 RR | AUCAUAGAG | ATTTTTCTCT | Chr11:5255052:5255071 | 0.10 | + |
| HBG2 | GAUACCAGG | AATTTATTC | | | |
| Promoter-17 | AC | T | | | |
| AsCpf1 RR | ACCAUAGAA | TTCTCCCAT | Chr11:5250162:5250181 | 1.40 | + |
| HBG1 | GAUACCAGG | CATAGAGGA | | | |
| Promoter-18 | AC | TA | | | |
| AsCpf1 RR | CAGUACCUG | ATCATAGAG | Chr11:5250169:5250188 | 7.88 | + |
| HBG1 | CCAAAGAAC | GATACCAGG | | | |
| Promoter-19 | AU | AC | | | |
| AsCpf1 RR | UAGUAUCUG | ACCATAGAA | Chr11:5255097:5255116 | 13.03 | + |
| HBG2 | GUAAAGAGC | GATACCAGG | | | |
| Promoter-20 | AU | AC | | | |
| AsCpf1 RR | UCAAUGCAA | CAGTACCTG | Chr11:5250714:5250733 | 13.31 | − |
| HBG1 | AUAUCUGUC | CCAAAGAAC | | | |
| Promoter-21 | UG | AT | | | |
| AsCpf1 RR | CUCUUGGGG | TAGTATCTG | Chr11:5255645:5255664 | 4.07 | − |
| HBG2 | GCCCCUUCC | GTAAAGAGC | | | |
| Promoter-22 | CC | AT | | | |
| AsCpf1 RVR | GAUUCAGUC | TCAATGCAA | Chr11:5250023:5250042 | 0.15 | − |
| HBG1 | AUUCCAAUU | ATATCTGTC | | | |
| Promoter-1 | UU | TG | | | |
| AsCpf1 RVR | AUUCAGUCA | CTCTTGGGG | Chr11:5250051:5250070 | 1.09 | − |
| HBG1 | UUCCAAUUU | GCCCCTTCC | | | |
| Promoter-2 | UU | CC | | | |
| AsCpf1 RVR | AAAAAAAUU | AAAAAAATT | Chr11:5250069:5250088 | *** | − |
| HBG1 | AGCAGUAUC | AGCAGTATC | | | |
| Promoter-3 | CU | CT | | | |
| AsCpf1 RVR | GCCUUUGCC | GCCTTTGCC | Chr11:5250093:5250112 | 3.96 | + |
| HBG1 | UUGUUCCGA | TTGTTCCGA | | | |
| Promoter-4 | UU | TT | | | |
| AsCpf1 RVR | AAAAAAAUU | AAAAAAATT | Chr11:5254997:5255016 | *** | - |
| HBG2 | AAGCAGCAG | AAGCAGCA | | | |
| Promoter-5 | UA | GTA | | | |
| AsCpf1 RVR | CUCAGUAAA | CTCAGTAAA | Chr11:5250693:5250712 | 5.32 | + |
| HBG1 | GAUGAUGGU | GATGATGGT | | | |
| Promoter-6 | AG | AG | | | |
| AsCpf1 RVR | ACUGGAUAA | ACTGGATAA | Chr11:5255567:5255586 | 9.78 | + |
| HBG2 | GUGUAUGUG | GTGTATGTG | | | |
| Promoter-7 | CU | CT | | | |
| AsCpf1 RVR | UGCUGGCUG | TGCTGGCTG | Chr11:5250652:5250671 | 0.24 | + |
| HBG2 | AUGACCCAG | ATGACCCAG | | | |
| Promoter-8 | GG | GG | | | |
| AsCpf1 RVR | CUCGGUAAA | CTCGGTAAA | Chr11:5255624:5255643 | 5.75 | + |
| HBG2 | GAUGAUGGU | GATGATGGT | | | |
| Promoter-9 | AG | AG | | | |
| AsCpf1 RVR | UGGUAAAGA | TGGTAAAGA | Chr11:5255638:5255657 | 8.55 | − |
| HBG2 | GCAUUCUAC | GCATTCTAC | | | |
| Promoter-10 | CA | CA | | | |

*the gRNA ID name provides the particular Cpf1 molecule used in the RNP complex
**NCBI Reference Sequence NC_000011, the coordinates are reported using the One-based coordinate system, “Homo sapiens chromosome 11, GRCh38.p12 Primary Assembly,” (Version NC_000011.10).
***represents gRNAs that were not tested.

RNPs (5 μM) containing AsCpf1 protein, AsCpf1 RR protein, or AsCpf1 RVR complexed with single gRNAs from Table 19 (see gRNA ID name for the particular Cpf1 molecule used; FIG. 46 provides the registry identification numbers for the gRNAs) were delivered to mobilized peripheral blood (mPB) CD34+ cells using the Amaxa electroporator device (Lonza). After 72 hours, genomic DNA was extracted from cells and the level of insertions/deletions at the target site was then analyzed by Illumina sequencing (NGS) of the PCR amplified target site. The percentage of editing (indels=deletions and insertions) for each gRNA is shown in Table 19 above. In certain embodiments, Cpf1 RNPs comprising one or more of the gRNAs set forth in Table 19 (and FIG. 46) may be used to target the regions listed in Table 18 to induce HbF expression.

To generate RNP complexation, Cpf1 gRNA was diluted to 352 μM in 1×H150+Magnesium (28.4 μl for 10 nmoles) and transferred to a AB1400 PCR plate and placed in a PCR machine that was run on a slow-anneal protocol (90° C. to 25° C. with 2% ramp, followed by 4° C.). 5 μl of 352 uM Annealed Guide was added to AB1400L PCR Plate and Cpf1 was diluted to 176 μM in 1×HG300, and 5 μl of 176 uM Cpf1 was added to the 5 μl 1352 uM Annealed Guide to yield 10 μM RNP.

To introduce RNPs into adult HSCs by Lonza nucleofection, 130 μl Complete HSC media with necessary growth factors was added to CellStar plates 130 μl and placed in a 37° C. incubator until needed. Adult HSCs were counted on Countess and based on the following formula, 50K cells/well: 2.50e6 cells/ml (10.5e6 cells for 2 plate bioreps), and sufficient cells were pipetted to cover 2 bioreps (2 plates) into 15 ml or 50 ml conical tubes. 1 tube (2 plate bioreps worth) was then spun down at 1000 rpm for 5 minutes in Beckman centrifuge. The media was removed and the cells were resuspended in in P2 solution (4.2 mls for complete plates). Using Mantis with Large Volume dispense chip, 20 μl of cell solution was dispenses per well of a Nucleocuvette plate. Using Biomek with P50 tips, 2 μl of RNP were transferred to Nucleocuvette plates containing cells and mixed by pipetting up and down. The plate was then placed in Amaxa shuttle and the following program was run, Cpf1: CA-137/Solution P2. Using Biomek with P50 tips, cells were transferred from Nucleocuvette plates to pre-warmed CellStar plates with media and incubated at 37° C. for 72 hours. Genomic DNA was extracted from cells using a DNAdvance kit according to manufacturer's instructions. The insertions/deletions relative to hg38 reference genome were quantified using NGS analysis of target site.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments described herein. Such equivalents are intended to be encompassed by the following claims.

1. An isolated cell comprising a modification in an HBG gene sequence or a BCL11a gene sequence generated by the delivery of a RNP complex comprising a CRISPR from Prevotella and Franciscella 1 (Cpf1) RNA-guided nuclease and a gRNA molecule that targets the HBG gene sequence or the BCL11a gene sequence.

2. A population of CD34+ cells or hematopoietic stem cells (HSCs), with one or more cells comprising a disruption in the cis-regulatory region of the HBG gene, wherein the disruption is generated using an RNP complex comprising a CRISPR/Cpf1 RNA-guided nuclease and a gRNA that targets the cis-regulatory region of the HBG gene.

3. The population of cells of claim 2, wherein the cis-regulatory region includes the CAAT box of the HBG gene promoter.

4. A method of treating or alleviating the symptoms of a hemoglobinopathy in a subject in need thereof, comprising administering a population of cells according to claim 2 or 3 to the subject.

5. The method of claim 4, wherein the populations of cells have an increase in the expression of fetal hemoglobin expression compared to an unmodified cell population or result in increased expression of fetal hemoglobin subsequent to administration, wherein the increase in the expression of fetal hemoglobin is an amount suitable to partially or completely alleviate the symptoms of a hemoglobinopathy.

6. An isolated T cell comprising a modification in a nucleic acid sequence generated by the delivery of a complex comprising a CRISPR from Prevotella and Franciscella 1 (Cpf1) RNA-guided nuclease and a gRNA molecule that targets the nucleic acid sequence, wherein the nucleic acid sequence is selected from the group consisting of: a portion of a FAS gene sequence, a portion of a BID gene sequence, a portion of a CTLA4 gene sequence, a portion of a PDCD1 gene sequence, a portion of a CBLB gene sequence, a portion of a PTPN6 gene sequence, a portion of a B2M gene sequence, a portion of a TRAC gene sequence, a portion of a CIITA gene sequence, a portion of a TRBC gene sequence and a combination thereof.

7. A population of T cells comprising a disruption in one or more genes selected from the group consisting of TRAC, TRBC, B2M and CIITA, wherein the disruption is generated using one or more RNP complexes comprising a CRISPR/CPf1 RNA-guided nuclease and a gRNA that targets a gene selected from the group consisting of TRAC, TRBC, CIITA and B2M, wherein at least 60% of the T cells of the population of T cells do not comprise a detectable level of MHC II receptor, TCR or B2M on the surface of the T cells.

8. The population of T cells according to claim 7, further comprising a chimeric antigen receptor (CAR) or engineered T cell receptor (eTCR) inserted at the disrupted TRAC locus.

9. The isolated T cell or population of T cells of claim 6, 7 or 8, wherein the T-cell is CD8⁺ T cell, a CD8⁺ naïve T cell, a CD4⁺ central memory T cell, a CD8⁺ central memory T cell, a CD4⁺ effector memory T cell, a CD4⁺ effector memory T cell, a CD4⁺ T cell, a CD4⁺ stem cell memory T cell, a CD8⁺ stem cell memory T cell, a CD4⁺ helper T cell, a regulatory T cell, a cytotoxic T cell, a natural killer T cell, a CD4+ naïve T cell, a TH17 CD4⁺ T cell, a TH1 CD4⁺ T cell, a TH2 CD4⁺ T cell, a TH9 CD4⁺ T cell, a CD4⁺ Foxp3⁺ T cell, a CD4⁺ CD25⁺ CD127⁻ T cell or a CD4⁺ CD25⁺ CD127⁻ Foxp3⁺ T cell.

10. An isolated CRISPR from Prevotella and Franciscella 1 (Cpf1) RNA-guided nuclease comprising a nuclear localization signal (NLS), wherein the Cpf1 RNA-guided nuclease comprises one or more NLS sequences at or near the N-terminus of the nuclease, one or more NLS sequences at or near the C-terminus of the nuclease or one or more NLS sequences at or near the N-terminus and C-terminus of the nuclease.

11. The isolated Cpf1 RNA-guided nuclease of claim 10, wherein the NLS sequences are selected from the group consisting of: the nucleoplasmin NLS (nNLS) (SEQ ID NO: 1) and the simian virus 40 “SV40” NLS (sNLS) (SEQ ID NO: 2).

12. An isolated CRISPR from Prevotella and Franciscella 1 (Cpf1) RNA-guided nuclease comprising a deletion or substitution of a cysteine amino acid, wherein the Cpf1 RNA-guided nuclease comprises a deletion or substitution at C65, C205, C334, C379, C608, C674, C1025, or C1248 of the wild type AsCpf1 amino acid sequence, and wherein the substitution is selected from the group consisting of C65S/A, C205S/A, C334S/A, C379S/A, C608S/A, C674S/A, and C1025S/A.

13. An isolated nucleic acid encoding a Cpf1 RNA-guided nuclease of any one of claims 10-12.

14. A method of modifying one or more target sequences of interest in a population of HSCs or T cells, comprising contacting the populations of cells ex vivo or in vitro with one or more RNP complexes comprising:
(a) a gRNA molecule complementary to the target sequence of interest; and
(b) a Cpf1 RNA-guided nuclease of any one of claims 10-12,
wherein the one or more RNP complexes modifies the one or more target sequences of interest in the population of cells.

15. The method of claim 14, wherein at least about 10%, at least about 20%, at least about 30%, at least about 40%, at least about 50%, at least about 60%, at least about 70%, at least about 80% or at least about 90% of the cells in the population of cells comprise a productive indel.

16. The method of any one of claims 14-15, wherein the target nucleic acid sequence is selected from the group consisting of: a portion of a B2M gene sequence, a portion of a TRAC gene sequence, a portion of a CIITA gene sequence, a portion of a TRBC gene sequence and a combination thereof.

17. A method of administering a population of cells to a subject, wherein the population of cells comprise a modification in an HBG gene sequence or a BCL11a gene sequence generated by the delivery of a complex comprising a CRISPR from Prevotella and Franciscella 1 (Cpf1) RNA-guided nuclease and a gRNA molecule that targets the HBG gene sequence or the BCL11a gene sequence.

18. The method of claim 17, wherein the subject suffers from a hemoglobinopathy.

19. The method of claim 17 or 18, wherein the population of cells comprise hematopoietic stem cells (HSCs) or a human umbilical cord blood-derived erythroid progenitor (HUDEP) cells.

20. A method of administering a population of T cells to a subject, wherein the population of cells comprise a modification in a gene selected from the group consisting of TRAC, TRBC, CIITA and B2M generated by the delivery of a complex comprising a CRISPR from Prevotella and Franciscella 1 (Cpf1) RNA-guided nuclease and a gRNA molecule that targets the gene.

21. The method of claim 20, wherein the subject suffers from cancer or an autoimmune disorder.

22. The method of any one of claims 17-21, wherein at least about 5%, at least about 10%, at least about 20%, at least about 30%, at least about 40%, at least about 50%, at least about 60%, at least about 70%, at least about 80% or at least about 90% of the cells in the population of cells comprise a productive indel.

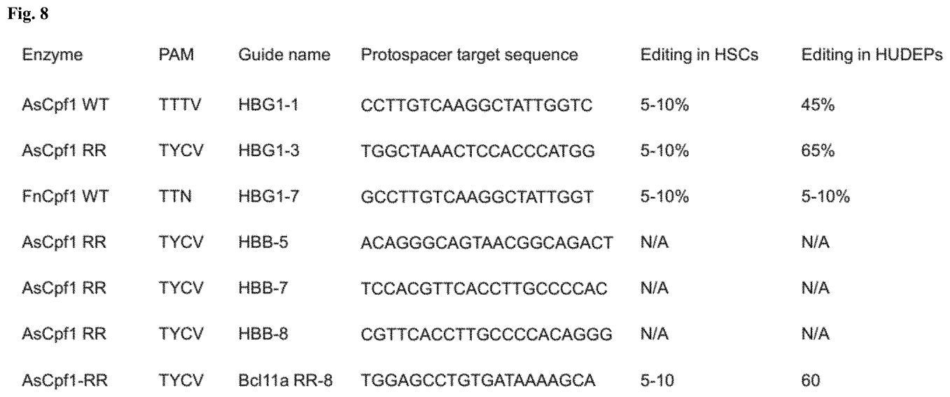

23. A gRNA molecule for a CRISPR from Prevotella and Franciscella 1 (Cpf1) RNA-guided nuclease comprising a first targeting domain that is complementary to a target sequence, wherein the target sequence is a HBG gene sequence or a BCL11a gene sequence, and wherein the gRNA molecule comprises a sequence that is the same, or differs by no more than 3 nucleotides from, than a sequence provided in FIG. 6, FIG. 7, FIG. 8, FIG. 46 and Table 19.

24. The gRNA molecule of claim 23, wherein (a) a CRISPR/Cpf1 system comprising the gRNA molecule is introduced into a cell, an indel is formed at or near the target sequence complementary to the first targeting domain of the gRNA molecule; and/or (b) when a CRISPR/Cpf1 system comprising the gRNA molecule is introduced into a cell, a deletion is created in a sequence complementary to the gRNA first targeting domain in the HBG1 promoter region or a HBG2 promoter region.

25. The gRNA molecule of claim 23, wherein when a CRISPR system comprising the gRNA molecule is introduced into a population of cells:
(a) expression of fetal hemoglobin is increased in the population of cells or its progeny relative to the level of expression of fetal hemoglobin in a population of cells to which the gRNA molecule was not introduced or a population of its progeny; and/or
(b) results in an increase in the expression of fetal hemoglobin in an amount suitable to partially or completely alleviate the symptoms of a hemoglobinopathy.

26. A composition comprising the gRNA molecule of any one of claims 23-25.

27. The composition of claim 26, further comprising a CRISPR from Prevotella and Franciscella 1 (Cpf1) RNA-guided nuclease.

28. A composition comprising a ribonucleoprotein (RNP) complex comprising the composition of claim 27.

29. The gRNA molecule of any one of claims 23-25, or the composition of any one of claims 26-28, for use in treating a subject suffering from a hemoglobinopathy.

30. A gRNA molecule for a CRISPR from Prevotella and Franciscella 1 (Cpf1) RNA-guided nuclease comprising a first targeting domain that is complementary to a target sequence, wherein the target sequence is selected from the group consisting of a portion of a B2M gene sequence, a portion of a TRAC gene sequence, a portion of a CIITA gene sequence, a portion of a TRBC gene sequence and a combination thereof, and wherein the gRNA molecule comprises a sequence that is the same, or differs by no more than 3 nucleotides from, than a sequence provided in Tables 2-9.

31. The gRNA molecule of claim 30, wherein (a) a CRISPR/Cpf1 system comprising the gRNA molecule is introduced into a cell, an indel is formed at or near the target sequence complementary to the first targeting domain of the gRNA molecule; and/or (b) when a CRISPR/Cpf1 system comprising the gRNA molecule is introduced into a cell, a deletion is created in a sequence complementary to the gRNA first targeting domain in the B2M gene sequence, the TRAC gene sequence, the CIITA gene sequence or the TRBC gene sequence.

32. A composition comprising the gRNA molecule of claim 30 or 31.

33. The composition of claim 32, further comprising a CRISPR from Prevotella and Franciscella 1 (Cpf1) RNA-guided nuclease.

34. The gRNA molecule of claim 30 or 31, or the composition of claim 32 or 34, for use in treating a subject suffering from cancer.

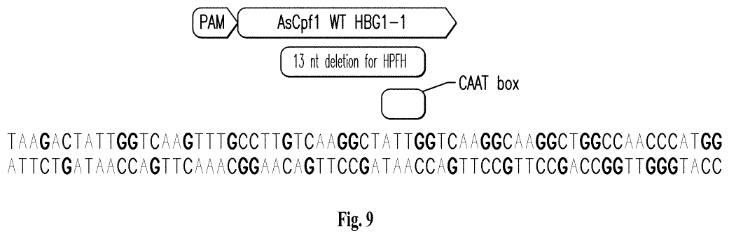

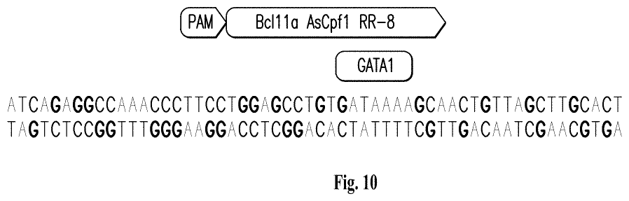

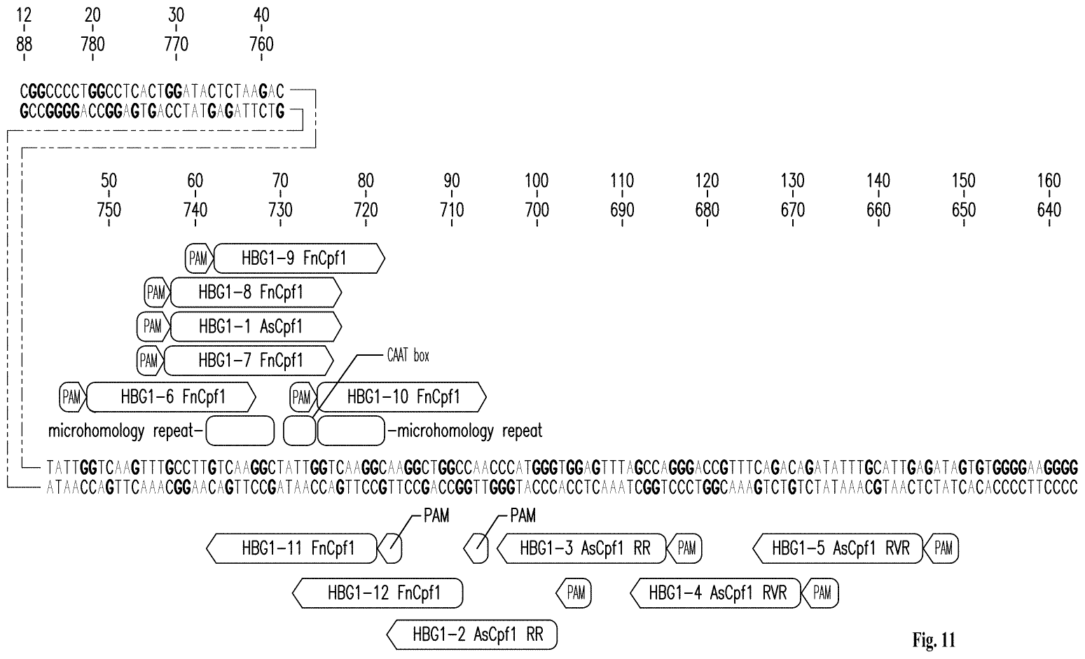

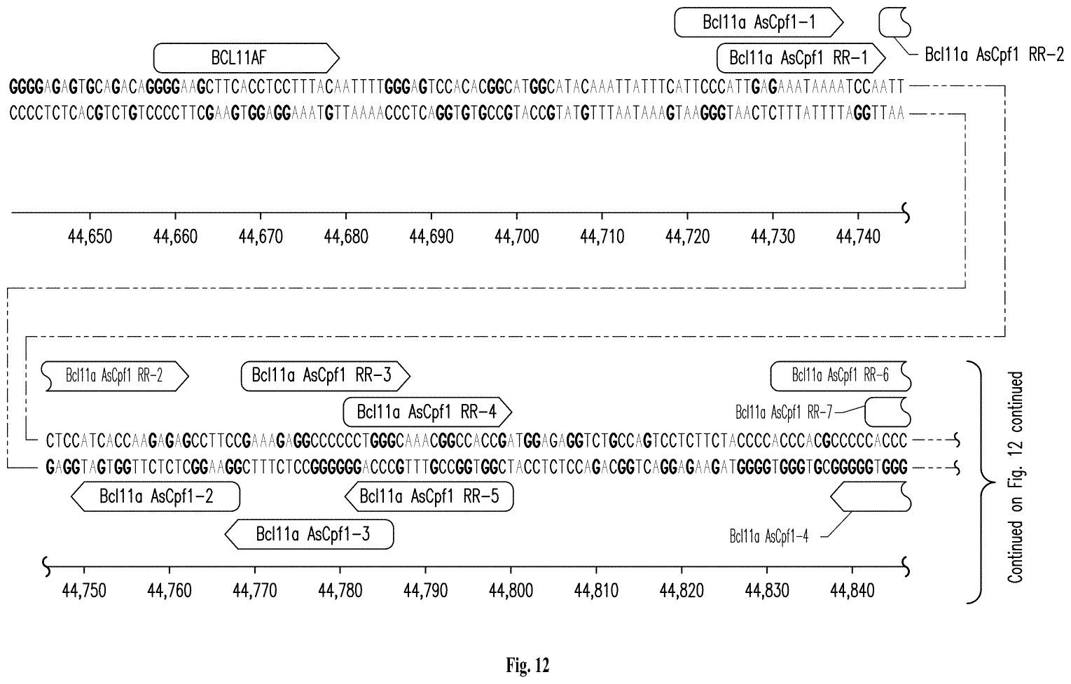

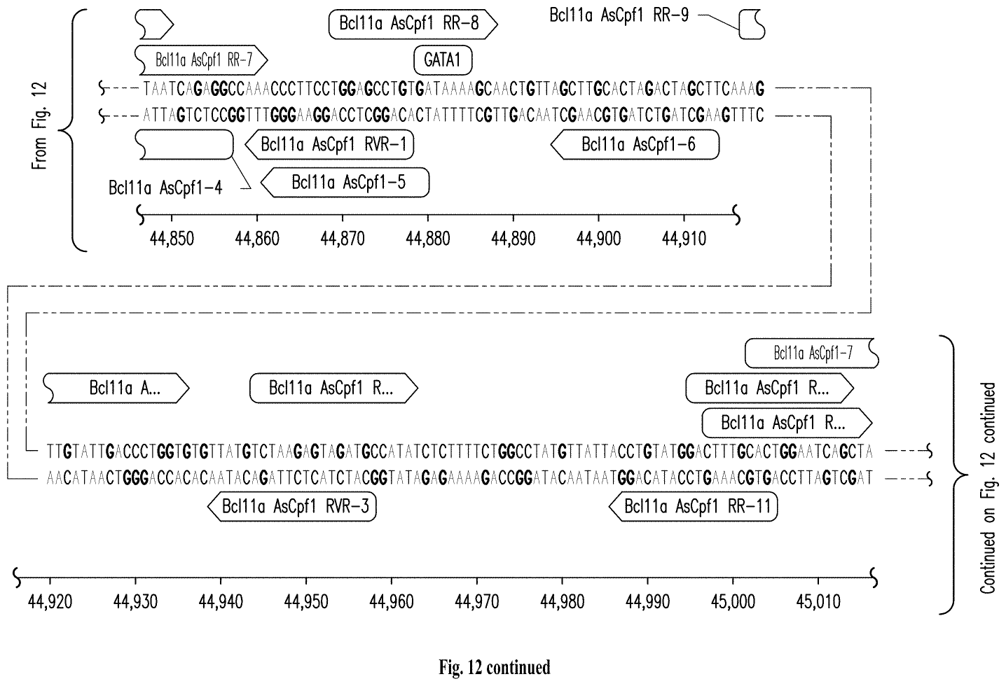

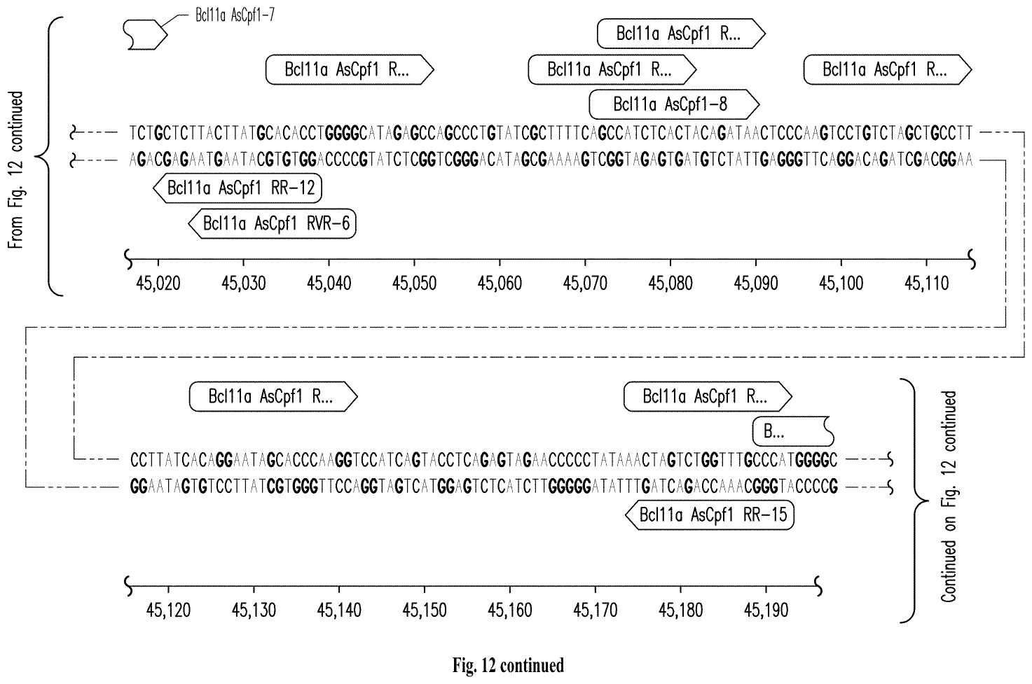

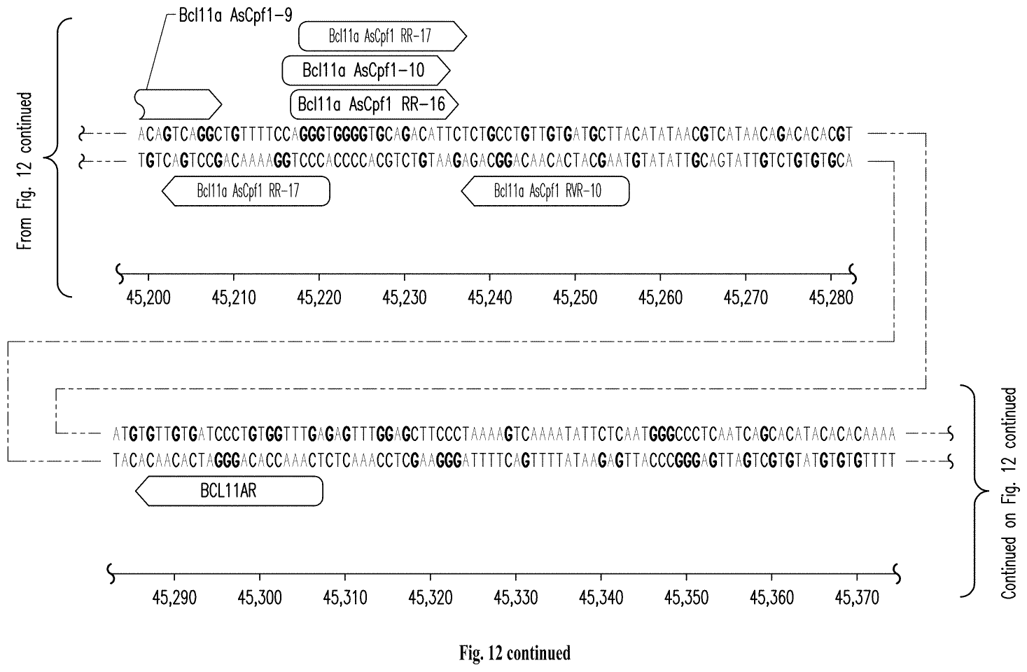

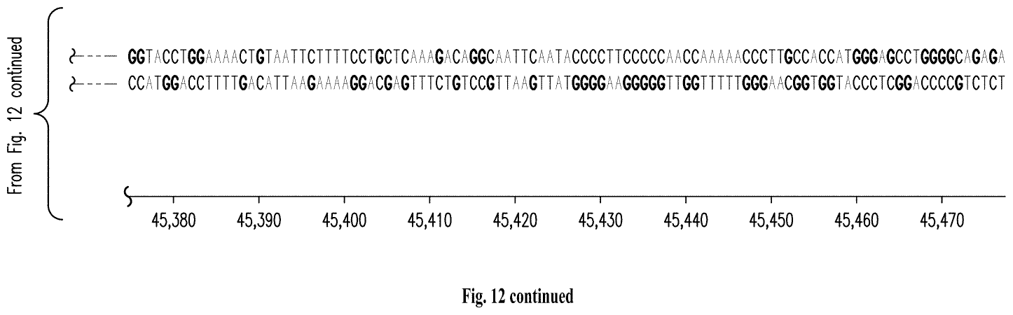

35. A genome editing system, the genome editing system comprising one or more RNP complexes comprising:
(a) a gRNA molecule, wherein the gRNA molecule comprises a sequence provided in FIG. 6, FIG. 7, FIG. 8, FIG. 9, FIG. 10, FIG. 11, FIG. 12, FIG. 46 and Tables 2-9 and 19; and
(b) a Cpf1 RNA-guided nuclease of any one of claims 10-12.

36. An assay for evaluating CRISPR/Cpf1-mediated editing of a target nucleic acid sequence and/or modulation of expression of a target nucleic acid sequence by a test Cpf1 RNA-guided nuclease comprising:
(a) determining the activity of the test Cpf1 RNA-guided nuclease with respect to the editing and/or modulation of expression of a target nucleic acid sequence comprising a matched site target nucleic acid sequence;
(b) comparing the activity of the test Cpf1 RNA-guided nuclease to the activity of a control RNA-guided nuclease with respect to the editing and/or modulation of expression of the target nucleic acid sequence comprising the matched site target nucleic acid sequence.


SEQUENCE LISTING (123 sequences)

SEQ ID NO: 1 - protein, 16 aa, Artificial Sequence
  the nucleoplasmin NLS (nNLS)
KRPAATKKAGQAKKKK

SEQ ID NO: 2 - protein, 7 aa, Artificial Sequence
  simian virus 40 “SV40” NLS
PKKKRKV

SEQ ID NO: 3 - protein, 1332 aa, Artificial Sequence
  Asp Cpf1 NLS v1
MKHHHHHHMTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNKRPAATKKAGQAKKKKG

SEQ ID NO: 4 - protein, 1324 aa, Artificial Sequence
  His-AsCpf1-sNLS
MKHHHHHHMTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGSPKKKRKV

SEQ ID NO: 5 - protein, 1333 aa, Artificial Sequence
  His-AsCpf1-sNLS-sNLS
MKHHHHHHMTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGSPKKKRKVGSPKKKRKV

SEQ ID NO: 6 - protein, 1326 aa, Artificial Sequence
  His-sNLS-AsCpf1
MKHHHHHHGSPKKKRKVGSMTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 7 - protein, 1333 aa, Artificial Sequence
  His-sNLS-sNLS-AsCpf1
MKHHHHHHGSPKKKRKVGSPKKKRKVMTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 8 - protein, 1327 aa, Artificial Sequence
  sNLS-sNLS-AsCpf1
MKGSPKKKRKVGSPKKKRKVMTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 9 - protein, 1332 aa, Artificial Sequence
  His-sNLS-AsCpf1-sNLS
MKHHHHHHMPKKKRKVMTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGPKKKRKVG

SEQ ID NO: 10 - protein, 1352 aa, Artificial Sequence
  His-sNLS-sNLS-AsCpf1-sNLS-sNLS
MGHHHHHHGSPKKKRKVGSPKKKRKVGSTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAARLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGSPKKKRKVGSPKKKRKV

SEQ ID NO: 11 - protein, 1332 aa, Artificial Sequence
  His-AsCpf1-nNLS Cys-less
MKHHHHHHMTQFEGFTNLYQVSKTLRFELIPQGKTLKHIQEQGFIEEDKARNDHYKELKPIIDRIYKTYADQSLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENSHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFSKYKTLLRNENVLETAEALFNELNSIDLTHIFISHKKLETISSALSDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKSSTQLKAVTAHFQTHTTPILLSNNFIEPLEITKEIYDLNNPEKEPKKFQTAYAKKTGDQKGYREALSKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNSLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVSFDSRFQNPEWPMDADANGAYHIALKGQLLLNHLKESKDLKLQNGISNQDWLAYIQELRNKRPAATKKAGQAKKKKG

SEQ ID NO: 12 - protein, 1332 aa, Artificial Sequence
  His-AsCpf1-nNLS Cys-low
MKHHHHHHMTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFSKYKTLLRNENVLETAEALFNELNSIDLTHIFISHKKLETISSALSDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALSKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNKRPAATKKAGQAKKKKG

SEQ ID NO: 13 - DNA, 20 bp, Artificial Sequence
  Matched Site 1
gattgaaggaaaagttacaa

SEQ ID NO: 14 - DNA, 20 bp, Artificial Sequence
  Matched Site 5
ggatgccactaaaagggaaa

SEQ ID NO: 15 - DNA, 20 bp, Artificial Sequence
  Matched Site 11
gctatcactgccatgtctgg

SEQ ID NO: 16 - DNA, 20 bp, Artificial Sequence
  Matched Site 18
ggggaggtgacaccactgaa

SEQ ID NO: 17 - protein, 1307 aa, Artificial Sequence
  Synthetic
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 18 - protein, 1228 aa, Artificial Sequence
  Synthetic
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 19 - protein, 1206 aa, Artificial Sequence
  Synthetic
MYYESLTKQYPVSKTIRNELIPIGKTLDNIRQNNILESDVKRKQNYEHVKGILDEYHKQLINEALDNCTLPSLKIAAEIYLKNQKEVSDREDFNKTQDLLRKEVVEKLKAHENFTKIGKKDILDLLEKLPSISEDDYNALESFRNFYTYFTSYNKVRENLYSDKEKSSTVAYRLINENFPKFLDNVKSYRFVKTAGILADGLGEEEQDSLFIVETFNKTLTQDGIDTYNSQVGKINSSINLYNQKNQKANGFRKIPKMKMLYKQILSDREESFIDEFQSDEVLIDNVESYGSVLIESLKSSKVSAFFDALRESKGKNVYVKNDLAKTAMSNIVFENWRTFDDLLNQEYDLANENKKKDDKYFEKRQKELKKNKSYSLEHLCNLSEDSCNLIENYIHQISDDIENIIINNETFLRIVINEHDRSRKLAKNRKAVKAIKDFLDSIKVLERELKLINSSGQELEKDLIVYSAHEELLVELKQVDSLYNMTRNYLTKKPFSTEKVKLNFNRSTLLNGWDRNKETDNLGVLLLKDGKYYLGIMNTSANKAFVNPPVAKTEKVFKKVDYKLLPVPNQMLPKVFFAKSNIDFYNPSSEIYSNYKKGTHKKGNMFSLEDCHNLIDFFKESISKHEDWSKFGFKFSDTASYNDISEFYREVEKQGYKLTYTDIDETYINDLIERNELYLFQIYNKDFSMYSKGKLNLHTLYFMMLFDQRNIDDVVYKLNGEAEVFYRPASISEDELIIHKAGEEIKNKNPNRARTKETSTFSYDIVKDKRYSKDKFTLHIPITMNFGVDEVKRFNDAVNSAIRIDENVNVIGIDRGERNLLYVVVIDSKGNILEQISLNSIINKEYDIETDYHALLDEREGGRDKARKDWNTVENIRDLKAGYLSQVVNVVAKLVLKYNAIICLEDLNFGFKRGRQKVEKQVYQKFEKMLIDKLNYLVIDKSREQTSPKELGGALNALQLTSKFKSFKELGKQSGVIYYVPAYLTSKIDPTTGFANLFYMKCENVEKSKRFFDGFDFIRFNALENVFEFGFDYRSFTQRACGINSKWTVCTNGERIIKYRNPDKNNMFDEKVVVVTDEMKNLFEQYKIPYEDGRNVKDMIISNEEAEFYRRLYRLLQQTLQMRNSTSDGTRDYIISPVKNKREAYFNSELSDGSVPKDADANGAYNIARKGLWVLEQIRQKSEGEKINLAMTNAEWLEYAQTHLL

SEQ ID NO: 20 - DNA, 4059 bp, Artificial Sequence
  Synthetic
atgacccagttcgagggcttcaccaacctgtaccaggtgagcaagaccctgcggttcgagctgatcccccagggcaagaccctgaagcacatccaggagcagggcttcatcgaggaggacaaggcccggaacgaccactacaaggagctgaagcccatcatcgaccggatctacaagacctacgccgaccagtgcctgcagctggtgcagctggactgggagaacctgagcgccgccatcgacagctaccggaaggagaagaccgaggagacccggaacgccctgatcgaggagcaggccacctaccggaacgccatccacgactacttcatcggccggaccgacaacctgaccgacgccatcaacaagcggcacgccgagatctacaagggcctgttcaaggccgagctgttcaacggcaaggtgctgaagcagctgggcaccgtgaccaccaccgagcacgagaacgccctgctgcggagcttcgacaagttcaccacctacttcagcggcttctacgagaaccggaagaacgtgttcagcgccgaggacatcagcaccgccatcccccaccggatcgtgcaggacaacttccccaagttcaaggagaactgccacatcttcacccggctgatcaccgccgtgcccagcctgcgggagcacttcgagaacgtgaagaaggccatcggcatcttcgtgagcaccagcatcgaggaggtgttcagcttccccttctacaaccagctgctgacccagacccagatcgacctgtacaaccagctgctgggcggcatcagccgggaggccggcaccgagaagatcaagggcctgaacgaggtgctgaacctggccatccagaagaacgacgagaccgcccacatcatcgccagcctgccccaccggttcatccccctgttcaagcagatcctgagcgaccggaacaccctgagcttcatcctggaggagttcaagagcgacgaggaggtgatccagagcttctgcaagtacaagaccctgctgcggaacgagaacgtgctggagaccgccgaggccctgttcaacgagctgaacagcatcgacctgacccacatcttcatcagccacaagaagctggagaccatcagcagcgccctgtgcgaccactgggacaccctgcggaacgccctgtacgagcggcggatcagcgagctgaccggcaagatcaccaagagcgccaaggagaaggtgcagcggagcctgaagcacgaggacatcaacctgcaggagatcatcagcgccgccggcaaggagctgagcgaggccttcaagcagaagaccagcgagatcctgagccacgcccacgccgccctggaccagcccctgcccaccaccctgaagaagcaggaggagaaggagatcctgaaaagccagctggacagcctgctgggcctgtaccacctgctggactggttcgccgtggacgagagcaacgaggtggaccccgagttcagcgcccggctgaccggcatcaagctggagatggagcccagcctgagcttctacaacaaggcccggaactacgccaccaagaagccctacagcgtggagaagttcaagctgaacttccagatgcccaccctggccagcggctgggacgtgaacaaggagaagaacaacggcgccatcctgttcgtgaagaacggcctgtactacctgggcatcatgcccaagcagaagggccggtacaaggccctgagcttcgagcccaccgagaagaccagcgagggcttcgacaagatgtactacgactacttccccgacgccgccaagatgatccccaagtgcagcacccagctgaaggccgtgaccgcccacttccagacccacaccacccccatcctgctgagcaacaacttcatcgagcccctggagatcaccaaggagatctacgacctgaacaaccccgagaaggagcccaagaagttccagaccgcctacgccaagaagaccggcgaccagaagggctaccgggaggccctgtgcaagtggatcgacttcacccgggacttcctgagcaagtacaccaagaccaccagcatcgacctgagcagcctgcggcccagcagccagtacaaggacctgggcgagtactacgccgagctgaaccccctgctgtaccacatcagcttccagcggatcgccgagaaggagatcatggacgccgtggagaccggcaagctgtacctgttccagatctacaacaaggacttcgccaagggccaccacggcaagcccaacctgcacaccctgtactggaccggcctgttcagccccgagaacctggccaagaccagcatcaagctgaacggccaggccgagctgttctaccggcccaagagccggatgaagcggatggcccaccggctgggcgagaagatgctgaacaagaagctgaaggaccagaagacccccatccccgacaccctgtaccaggagctgtacgactacgtgaaccaccggctgagccacgacctgagcgacgaggcccgggccctgctgcccaacgtgatcaccaaggaggtgagccacgagatcatcaaggaccggcggttcaccagcgacaagttcttcttccacgtgcccatcaccctgaactaccaggccgccaacagccccagcaagttcaaccagcgggtgaacgcctacctgaaggagcaccccgagacccccatcatcggcatcgaccggggcgagcggaacctgatctacatcaccgtgatcgacagcaccggcaagatcctggagcagcggagcctgaacaccatccagcagttcgactaccagaagaagctggacaaccgggagaaggagcgggtggccgcccggcaggcctggagcgtggtgggcaccatcaaggacctgaagcagggctacctgagccaggtgatccacgagatcgtggacctgatgatccactaccaggccgtggtggtgctggagaacctgaacttcggcttcaagagcaagcggaccggcatcgccgagaaggccgtgtaccagcagttcgagaagatgctgatcgacaagctgaactgcctggtgctgaaggactaccccgccgagaaggtgggcggcgtgctgaacccctaccagctgaccgaccagttcaccagcttcgccaagatgggcacccagagcggcttcctgttctacgtgcccgccccctacaccagcaagatcgaccccctgaccggcttcgtggaccccttcgtgtggaagaccatcaagaaccacgagagccggaagcacttcctggagggcttcgacttcctgcactacgacgtgaagaccggcgacttcatcctgcacttcaagatgaaccggaacctgagcttccagcggggcctgcccggcttcatgcccgcctgggacatcgtgttcgagaagaacgagacccagttcgacgccaagggcacccccttcatcgccggcaagcggatcgtgcccgtgatcgagaaccaccggttcaccggccggtaccgggacctgtaccccgccaacgagctgatcgccctgctggaggagaagggcatcgtgttccgggacggcagcaacatcctgcccaagctgctggagaacgacgacagccacgccatcgacaccatggtggccctgatccggagcgtgctgcagatgcggaacagcaacgccgccaccggcgaggactacatcaacagccccgtgcgggacctgaacggcgtgtgcttcgacagccggttccagaaccccgagtggcccatggacgccgacgccaacggcgcctaccacatcgccctgaagggccagctgctgctgaaccacctgaaggagagcaaggacctgaagctgcagaacggcatcagcaaccaggactggctggcctacatccaggagctgcggaacaagcggcccgccgccaccaagaaggccggccaggccaagaagaagaagggcagctacccctacgacgtgcccgactacgcctacccctacgacgtgcccgactacgcctacccctacgacgtgcccgactacgcctga

SEQ ID NO: 21 - DNA, 3822 bp, Artificial Sequence
  Synthetic
atgagcaagctggagaagttcaccaactgctacagcctgagcaagaccctgcggttcaaggccatccccgtgggcaagacccaggagaacatcgacaacaagcggctgctggtggaggacgagaagcgggccgaggactacaagggcgtgaagaagctgctggaccggtactacctgagcttcatcaacgacgtgctgcacagcatcaagctgaagaacctgaacaactacatcagcctgttccggaagaagacccggaccgagaaggagaacaaggagctggagaacctggagatcaacctgcggaaggagatcgccaaggccttcaagggcaacgagggctacaagagcctgttcaagaaggacatcatcgagaccatcctgcccgagttcctggacgacaaggacgagatcgccctggtgaacagcttcaacggcttcaccaccgccttcaccggcttcttcgacaaccgggagaacatgttcagcgaggaggccaagagcaccagcatcgccttccggtgcatcaacgagaacctgacccggtacatcagcaacatggacatcttcgagaaggtggacgccatcttcgacaagcacgaggtgcaggagatcaaggagaagatcctgaacagcgactacgacgtggaggacttcttcgagggcgagttcttcaacttcgtgctgacccaggagggcatcgacgtgtacaacgccatcatcggcggcttcgtgaccgagagcggcgagaagatcaagggcctgaacgagtacatcaacctgtacaaccagaagaccaagcagaagctgcccaagttcaagcccctgtacaagcaggtgctgagcgaccgggagagcctgagcttctacggcgagggctacaccagcgacgaggaggtgctggaggtgttccggaacaccctgaacaagaacagcgagatcttcagcagcatcaagaagctggagaagctgttcaagaacttcgacgagtacagcagcgccggcatcttcgtgaagaacggccccgccatcagcaccatcagcaaggacatcttcggcgagtggaacgtgatccgggacaagtggaacgccgagtacgacgacatccacctgaagaagaaggccgtggtgaccgagaagtacgaggacgaccggcggaaaagcttcaagaagatcggcagcttcagcctggagcagctgcaggagtacgccgacgccgacctgagcgtggtggagaagctgaaggagatcatcatccagaaggtggacgagatctacaaggtgtacggcagcagcgagaagctgttcgacgccgacttcgtgctggagaaaagcctgaagaagaacgacgccgtggtggccatcatgaaggacctgctggacagcgtgaaaagcttcgagaactacatcaaggccttcttcggcgagggcaaggagaccaaccgggacgagagcttctacggcgacttcgtgctggcctacgacatcctgctgaaggtggaccacatctacgacgccatccggaactacgtgacccagaagccctacagcaaggacaagttcaagctgtacttccagaacccccagttcatgggcggctgggacaaggacaaggagaccgactaccgggccaccatcctgcggtacggcagcaagtactacctggccatcatggacaagaagtacgccaagtgcctgcagaagatcgacaaggacgacgtgaacggcaactacgagaagatcaactacaagctgctgcccggccccaacaagatgctgcccaaggtgttcttcagcaagaagtggatggcctactacaaccccagcgaggacatccagaagatctacaagaacggcaccttcaagaagggcgacatgttcaacctgaacgactgccacaagctgatcgacttcttcaaggacagcatcagccggtaccccaagtggagcaacgcctacgacttcaacttcagcgagaccgagaagtacaaggacatcgccggcttctaccgggaggtggaggagcagggctacaaggtgagcttcgagagcgccagcaagaaggaggtggacaagctggtggaggagggcaagctgtacatgttccagatctacaacaaggacttcagcgacaagagccacggcacccccaacctgcacaccatgtacttcaagctgctgttcgacgagaacaaccacggccagatccggctgagcggcggcgccgagctgttcatgcggcgggccagcctgaagaaggaggagctggtggtgcaccccgccaacagccccatcgccaacaagaaccccgacaaccccaagaagaccaccaccctgagctacgacgtgtacaaggacaagcggttcagcgaggaccagtacgagctgcacatccccatcgccatcaacaagtgccccaagaacatcttcaagatcaacaccgaggtgcgggtgctgctgaagcacgacgacaacccctacgtgatcggcatcgaccggggcgagcggaacctgctgtacatcgtggtggtggacggcaagggcaacatcgtggagcagtacagcctgaacgagatcatcaacaacttcaacggcatccggatcaagaccgactaccacagcctgctggacaagaaggagaaggagcggttcgaggcccggcagaactggaccagcatcgagaacatcaaggagctgaaggccggctacatcagccaggtggtgcacaagatctgcgagctggtggagaagtacgacgccgtgatcgccctggaggacctgaacagcggcttcaagaacagccgggtgaaggtggagaagcaggtgtaccagaagttcgagaagatgctgatcgacaagctgaactacatggtggacaagaaaagcaacccctgcgccaccggcggcgccctgaagggctaccagatcaccaacaagttcgagagcttcaagagcatgagcacccagaacggcttcatcttctacatccccgcctggctgaccagcaagatcgaccccagcaccggcttcgtgaacctgctgaagaccaagtacaccagcatcgccgacagcaagaagttcatcagcagcttcgaccggatcatgtacgtgcccgaggaggacctgttcgagttcgccctggactacaagaacttcagccggaccgacgccgactacatcaagaagtggaagctgtacagctacggcaaccggatccggatcttccggaaccccaagaagaacaacgtgttcgactgggaggaggtgtgcctgaccagcgcctacaaggagctgttcaacaagtacggcatcaactaccagcagggcgacatccgggccctgctgtgcgagcagagcgacaaggccttctacagcagcttcatggccctgatgagcctgatgctgcagatgcggaacagcatcaccggccggaccgacgtggacttcctgatcagccccgtgaagaacagcgacggcatcttctacgacagccggaactacgaggcccaggagaacgccatcctgcccaagaacgccgacgccaacggcgcctacaacatcgcccggaaggtgctgtgggccatcggccagttcaagaaggccgaggacgagaagctggacaaggtgaagatcgccatcagcaacaaggagtggctggagtacgcccagaccagcgtgaagcacaagcggcccgccgccaccaagaaggccggccaggccaagaagaagaagggcagctacccctacgacgtgcccgactacgcctacccctacgacgtgcccgactacgcctacccctacgacgtgcccgactacgcctga

SEQ ID NO: 22 - DNA, 3756 bp, Artificial Sequence
  Synthetic
atgtactacgagagcctgaccaagcagtaccccgtgagcaagaccatccggaacgagctgatccccatcggcaagaccctggacaacatccggcagaacaacatcctggagagcgacgtgaagcggaagcagaactacgagcacgtgaagggcatcctggacgagtaccacaagcagctgatcaacgaggccctggacaactgcaccctgcccagcctgaagatcgccgccgagatctacctgaagaaccagaaggaggtgagcgaccgggaggacttcaacaagacccaggacctgctgcggaaggaggtggtggagaagctgaaggcccacgagaacttcaccaagatcggcaagaaggacatcctggacctgctggagaagctgcccagcatcagcgaggacgactacaacgccctggagagcttccggaacttctacacctacttcaccagctacaacaaggtgcgggagaacctgtacagcgacaaggagaaaagcagcaccgtggcctaccggctgatcaacgagaacttccccaagttcctggacaacgtgaaaagctaccggttcgtgaagaccgccggcatcctggccgacggcctgggcgaggaggagcaggacagcctgttcatcgtggagaccttcaacaagaccctgacccaggacggcatcgacacctacaacagccaggtgggcaagatcaacagcagcatcaacctgtacaaccagaagaaccagaaggccaacggcttccggaagatccccaagatgaagatgctgtacaagcagatcctgagcgaccgggaggagagcttcatcgacgagttccagagcgacgaggtgctgatcgacaacgtggagagctacggcagcgtgctgatcgagagcctgaaaagcagcaaggtgagcgccttcttcgacgccctgcgggagagcaagggcaagaacgtgtacgtgaagaacgacctggccaagaccgccatgagcaacatcgtgttcgagaactggcggaccttcgacgacctgctgaaccaggagtacgacctggccaacgagaacaagaagaaggacgacaagtacttcgagaagcggcagaaggagctgaagaagaacaagagctacagcctggagcacctgtgcaacctgagcgaggacagctgcaacctgatcgagaactacatccaccagatcagcgacgacatcgagaacatcatcatcaacaacgagaccttcctgcggatcgtgatcaacgagcacgaccggagccggaagctggccaagaaccggaaggccgtgaaggccatcaaggacttcctggacagcatcaaggtgctggagcgggagctgaagctgatcaacagcagcggccaggagctggagaaggacctgatcgtgtacagcgcccacgaggagctgctggtggagctgaagcaggtggacagcctgtacaacatgacccggaactacctgaccaagaagcccttcagcaccgagaaggtgaagctgaacttcaaccggagcaccctgctgaacggctgggaccggaacaaggagaccgacaacctgggcgtgctgctgctgaaggacggcaagtactacctgggcatcatgaacaccagcgccaacaaggccttcgtgaacccccccgtggccaagaccgagaaggtgttcaagaaggtggactacaagctgctgcccgtgcccaaccagatgctgcccaaggtgttcttcgccaagagcaacatcgacttctacaaccccagcagcgagatctacagcaactacaagaagggcacccacaagaagggcaacatgttcagcctggaggactgccacaacctgatcgacttcttcaaggagagcatcagcaagcacgaggactggagcaagttcggcttcaagttcagcgacaccgccagctacaacgacatcagcgagttctaccgggaggtggagaagcagggctacaagctgacctacaccgacatcgacgagacctacatcaacgacctgatcgagcggaacgagctgtacctgttccagatctacaacaaggacttcagcatgtacagcaagggcaagctgaacctgcacaccctgtacttcatgatgctgttcgaccagcggaacatcgacgacgtggtgtacaagctgaacggcgaggccgaggtgttctaccggcccgccagcatcagcgaggacgagctgatcatccacaaggccggcgaggagatcaagaacaagaaccccaaccgggcccggaccaaggagaccagcaccttcagctacgacatcgtgaaggacaagcggtacagcaaggacaagttcaccctgcacatccccatcaccatgaacttcggcgtggacgaggtgaagcggttcaacgacgccgtgaacagcgccatccggatcgacgagaacgtgaacgtgatcggcatcgaccggggcgagcggaacctgctgtacgtggtggtgatcgacagcaagggcaacatcctggagcagatcagcctgaacagcatcatcaacaaggagtacgacatcgagaccgactaccacgccctgctggacgagcgggagggcggccgggacaaggcccggaaggactggaacaccgtggagaacatccgggacctgaaggccggctacctgagccaggtggtgaacgtggtggccaagctggtgctgaagtacaacgccatcatctgcctggaggacctgaacttcggcttcaagcggggccggcagaaggtggagaagcaggtgtaccagaagttcgagaagatgctgatcgacaagctgaactacctggtgatcgacaagagccgggagcagaccagccccaaggagctgggcggcgccctgaacgccctgcagctgaccagcaagttcaagagcttcaaggagctgggcaagcagagcggcgtgatctactacgtgcccgcctacctgaccagcaagatcgaccccaccaccggcttcgccaacctgttctacatgaagtgcgagaacgtggagaaaagcaagcggttcttcgacggcttcgacttcatccggttcaacgccctggagaacgtgttcgagttcggcttcgactaccggagcttcacccagcgggcctgcggcatcaacagcaagtggaccgtgtgcaccaacggcgagcggatcatcaagtaccggaaccccgacaagaacaacatgttcgacgagaaggtggtggtggtgaccgacgagatgaagaacctgttcgagcagtacaagatcccctacgaggacggccggaacgtgaaggacatgatcatcagcaacgaggaggccgagttctaccggcggctgtaccggctgctgcagcagaccctgcagatgcggaacagcaccagcgacggcacccgggactacatcatcagccccgtgaagaacaagcgggaggcctacttcaacagcgagctgagcgacggcagcgtgcccaaggacgccgacgccaacggcgcctacaacatcgcccggaagggcctgtgggtgctggagcagatccggcagaaaagcgagggcgagaagatcaacctggccatgaccaacgccgagtggctggagtacgcccagacccacctgctgaagcggcccgccgccaccaagaaggccggccaggccaagaagaagaagggcagctacccctacgacgtgcccgactacgcctacccctacgacgtgcccgactacgcctacccctacgacgtgcccgactacgcctga

SEQ ID NO: 23 - DNA, 2000 bp, Artificial Sequence
  Synthetic
tcaatagcccagtcggttttgttagatacattttatcgaatctgtaaagatattttataataagataatatcagcgcctagctgcggaattccactcagagaatacctctcctgaatatcagccttagtggcgttatacgatatttcacactctcaaaatcccgagtcagactatacccgcgcatgtttagtaaaggttgattctgagatctcgagtccaaaaaagatacccactactttaaagatttgcattcagttgttccatcggcctgggtagtaaagggggtatgctcgctccgagtcgatggaactgtaaatgttagccctgatacgcggaacatatcagtaacaatctttacctaatatggagtgggattaagcttcatagaggatatgaaacgctcgtagtatggcttcctacataagtagaattattagcaactaagatattaccactgcccaataaaagagattccacttagattcataggtagtcccaacaatcatgtctgaatactaaattgatcaattggactatgtcaaaattattttgaagaagtaatcatcaacttaggcgctttttagtgttaagagcgcgttattgccaaccgggctaaacctgtgtaactcttcaatattgtatataattataggcagaataagctatgagtgcattatgagataaacatagatttttgtccactcgaaatatttgaatttcttgatcctgggctagttcagccataagttttcactaatagttaggactaccaattacactacattcagttgctgaaattcacatcactgccgcaatatttatgaagctattattgcattaagacttaggagataaatacgaagttgatatatttttcagaatcagcgaaaagaccccctattgacattacgaattcgagtttaacgagcacataaatcaaacactacgaggttaccaagattgtatcttacattaatgctatccagccagccgtcatgtttaactggatagtcataattaatatccaatgatcgtttcacgtagctgcatatcgaggaagttgtataattgaaaacccacacattagaatgcatggtgcatcgctagggtttatcttatcttgctcgtgccaagagtgtagaaagccacatattgatacggaagctgcctaggaggttggtatatgttgattgtgctcaccatctcccttcctaatctcctagtgttaagtccaatcagtgggctggctctggttaaaagtaatatacacgctagatctctctactataatacaggctaagcctacgcgctttcaatgcactgattaccaacttagctacggccagccccatttaatgaattatctcagatgaattcagacattattctctacaaggacactttagagtgtcctgcggaggcataattattatctaagatggggtaagtccgatggaagacacagatacatcggactattcctattagccgagagtcaaccgttagaactcggaaaaagacatcgaagccggtaacctacgcactataaatttccgcagagacatatgtaaagttttattagaactggtatcttgattacgattcttaactctcatacgccggtccggaatttgtgactcgagaaaatgtaatgacatgctccaattgatttcaaaattagatttaaggtcagcgaactatgtttattcaaccgtttacaacgctattatgcgcgatggatggggccttgtatctagaaaccgaataataacatacctgttaaatggcaaacttagattattgcgattaattctcacttcagagggttatcgtgccgaattcctgactttggaataataaagttgatattgaggtgcaatatcaactacactggtttaacctttaaacacatggagtcaagttttcgctatgccagccggttatgcagctaggattaatattagagctcttttctaattcgtcctaataatctcttcac

SEQ ID NO: 24 - DNA, 2000 bp, Artificial Sequence
  Synthetic
aaaacgtactacgtccactaatatagtgctcagggcctttaaagttatgaacaggaatacggcgatgacgatagagatgtacaactcagtgcgaaccccagtgtatgtacaaaaagttactaattcactttactgttttgaggatgtacctgccaaaaagattcagattatcaaagtcagatctttatatgacggaacgcgcaaaggatcctattaggatgcgcctcaaaaagccatctaaaaagttcatgtattgagcttattagtaaaggtatcaacaaaaatgattccaccttatataaataagcttgatcccattaattgaataataaagaccgagtaatcacttttatgcatgtaacaaaaatcccgtttgcggctatgctacaacggtcatcccatagaatattatcatcgtacaagcccaagacccgatgctcaacattagagccaaataacgtgcacactcctaatatgagatgactgccgcttttaacaccagatctgttagttaggccacgcacttccaagtttatctagagtgcatgtctttatatatgttggtcccctgtaatgacttataatatttccttcgactgtgttgaacatctgtaacaataaagactaaagctctgggtatataaggttgcagtggtaccttattaggtccattatcgcagaatactgcggatggacaatcttgccaatttaattgactatctattagtttgcacaatataacgattcgtcttggacaaatttggcgagtgagccccttactcgctcaaaatgttacaattgccgagctcggagttgaatgattagttacatattatagaacacaatgcagatgtagttagacaagatgtgttgatgaatgtcaagtctgactggagtaaaggaacaagagcacccacctacgtatattgcgcattttaaatgtagcctcgactctaacacgtgcgacgtgagtcataattgtgcatgttattagatctatggaatgttgtttttttaattatcaaacgtacgtcaaaccgccaaactccgtgtgccatagagtatactcctgaagttcgaaattaggccataaagtctttcttgctggttgtgaaatgaaggggtgtttcataatttaactttgactgcttctgttgggacgacgtacccgttcgtttgtttgtcctactatttagtatcttaaaacagtccatttaccgttaatgttcttaacccttaaagatacaaacttagctctgtaatcaacttcaagacgtctttgacagaacgtctaagacccagatctgtgttagccaactcgtattcaatttcgtaccggtggacttcggcccctcacactgccattagttgatgctgaactttgtatttgctgggtaggatatataacgattttgcagatgtgtgtgctaagtatattgtcttagtgacggtccagcatataaaacacctacacaagaaggttattcttaatggttgattgaatattattaaattgttgcttttactttttcctcctacaaattgtcatgagctcaaatttgttgacctaaggtattaatattgtatcctacacggattgtgaacggtagggtcgtaacaatcgtactttacggcttaaaaattgtaagcaccttgccaggtagatgaaaacttaaaggatagaagtatagtaactcacatgcttgcggcagcatcgtagggcagaggtgtgatcttggtgattgaaattaaggggtaggatgatcggccgcatatatcggctactaggattagatagatgcaacgctttactttaatcaagtgacgtccgtataagtaagacatctaatggctgtatttttgtatacaagtataaggaaccggggagtctttatagcgacgcgtaattatatattccaaatcagttaagtggcgtcggttacgaaactaaagagagtgttcaagacgcaatgaagaatcgtgagcgtaattgttcgcgc

SEQ ID NO: 25 - DNA, 2000 bp, Artificial Sequence
  Synthetic
aaccctcgtgtccggtaaaacacgcttcgaatacaaaagattatataggtacggaaggctgggaatctttcttcgatggaactgagattatattccactgtaaccttattatgactatagatttccaacatacggatagattaataccgactgtagattccatacttgaactatgaagccgtacgagtacccatactataactaagactatgacacgtgtgaattcgtgtttatcatagtgcaaactcttgctattccacatgggagtttagaactcagctgttcctatacaattagcactacaaacccactaatatggatagcatgataccatctgaggaggatttggtgttaccatgttgtaatctaagaagtttcacaaaatcaacgttagataaacggcaatatacgcgcactaataatgaaccccaagatatcagttgaaaaattttcgatctcctctttaaattaacaaatattgcagagtaagtaccgaaattgtgacacaagtgccgtttgcccgtctttttcacagcctataaagttcagatctatatgggctcccacttaaccttcagatagataacaagttactggaagtgattctatcataatacaatcaactataacacatccaatgatatatctcgagaaagtcgtagtctagagctccttctattatccggtcttacctaaatagttatatttagttgcccatttaaaattggataggaggaggggtgctcatgatttaaaaaccaactgtgcatgcggttctttgatgtggatccaccttgcaaagcgctaaagataaaagtagtcactacaggaattcaacttccgtcgttgtcagctggcgcgggaacccatcttgtgtaaaaaactgtataaccagacacgtggactcgaccgagaaacagtcagaacctgtcacaagaaataatcttgattaaaggctttcacggcaaacggacctcttccctgctgaagtgtacgattgaatatccacatcgaaggtcaattaccctcatcttttacatggtcataagacaataatctcctatttggattaaaatccgcgcacgaaagataagagtggaatcgattgcattatcgagtttttaagccccatacccgacagatgtgtaaaaagtgtagtggtaatggcgtcaccaagacctatgcttctcataataataggacgtatgccctagctactgctaacggtcgctcttacaatactagctaaaagaaacaaatttgaaaagttatgtaggaagtcattggcggtgaaaaagtgagaaaaaaggtccccggagactgtgctttcatgttatcaaagtacatgccgagtgaagagtttgttttgatcaacttttattatctggagtcattatacgatattgccatggttccttggctgtccaaccaggggtcttttacaccagataatcttctactacactacacctcaggtacgattctttcgttatcaatcgactacaagattatagtgtctctaaggcgtgatgtaggttttccctcaatgacaaagactttacagcaatccggttcaatacgagaattaagtgtgcgagtaacagcaaagtaaaatctaacagaaaggagactcagaaaacaacctattgaggactgtaatatcaactcagcattattgtttactttaaaatctaataatcgtttcgaggatatgagcacggtatcctaacatcaagacaaataccacatcatctaaatacaactggttgcaatgagtcgaatcgcgaacaaataaagcaactataagcacgataaaccactgttatgggaatgataaacagtcttatgacgtggtctatctgtcgtaggtggtaaagccttctgaagatcactatccagttctggcctcaagaaccatttagacagccttttctaaacatgatcgttgctataaggaccggggacacctagacaaactcacggaagggataacttacatc

SEQ ID NO: 26 - DNA, 2000 bp, Artificial Sequence
  Synthetic
actgcttatataggaggtacaaacagatacaatccttagttaactagagagaatgctttttttcgaccgacacgcttataacttcactgggcatggtcaccatatttaggtaaaacaaactgctgcgctatatgtcgtacacatcctgagtgtaccaatatgtaggtggaaggcaagttcaatgagacgtcagttaccaagcaaatttacattctagcagttataaatgtattatgacgcagttcttgtggtgagcgatcatttacattaaaactttattcaagagcgtatattagcatatattttccggagagtgcactacgggccgaaatttaggctggaactccgcaaattggttacgaccctgtatacatagttcttattattaagtaaaatgtgtgaataaaacctacacgacgcgtcatatacgtaaaagtttatctcttgtagtaatcaactaaattaacttactactatctggtcgtccgtatgaccctgtgagcagattattttcgactcgacatctatgaattctacggcacgaaaagttggtaacttgtactgggttaaacaatgtgtattcgggagtctgcggaagaacgtttttaatgtaacttcctttgcaaaccaaaatttggtctattcaaactgacactagcgtaatctataccgcatgagatcctgacatgatcctatatctatgcgcataggtactcgcaccaataagtgggtcgtagaatttcacgtaactcaatgttgtctcctttcattttttgttaattcgagaaaactacaaaaatagttagtaaaatgctcaaggagtcaggtgctacctgtggaatacatctatgtccaatggaacttgctccctcggatgtgcgatttcgttgttcagttgggcctttaaggaatacagcaactccaactctttgattttaggtaagtatttgattcgcggaaagtacagtgtataatctgttatttgccaagacgtcatcgaaatcgagtgtatcgagatcagaccatcgcgctatcgcaagatatgaagagcatagacagatcacgatgccaatcagtgtcgatggtgcgaagacgcagcccctgtgatcaaatcgtccgtttctcgatttactagcggaaaacaaaaacgaagcggtgaataccctgcgagctaatgtctttacccggttatacgagctgataactcggaaaatgctaatatcgaggctgcgcacttaaaaaaatactttaataatattaataagcatagctgtatcataacttaaaattctactgtatgatttagaatctaacagtgttaacgatctacagaccgcactaagatgaagacggactaatctcctccctaattttccttgttgattagcaaagggagatccttttgttatttgaggtttacgagaaagatgtaagagtcgaaataattacgtaaacctcatagtcgtcacctagagcaactataacatgaaccactcgccttggttaaatataaaataacttcttctctgtaacattgttgcacacaagcgagcgacaaaatttcacaacatttgttgcgtagataatattactgcatcatttttgcgtcagagtgaatgtcacttatataactaggaaaaattagtaggatagctcttgcggttgagagtaatgtcgactgaatcgaccgccatagatggtagagggagtgattcaaatagattaatgtatgcgctccatctataaggacggacaaggatcaatgttcccttatacttagctaacaggaccctctccgaaggtctgataatgcactcatataagcatcgatgcgtcctgagtagaaaaatctttacaaacttttaatagataagttatcttggaggtgctatctattcaaatctctgaacagatctgcggcatgataatgtctttgtaccggtgtgaataatgtgagtcagacgtctgtgcgaagtgggaaccgaaatcttttaatcatt

SEQ ID NO: 27 - DNA, 2000 bp, Artificial Sequence
  Synthetic
gattcggtcgcgttccataatcgaacccttaagcccatcttccagctgttaacgttatgtaccatcttacctcaatgtcagcgatctatgaggttcatgtttttggtggattaaaaaacttctttatagtggtttagacagaacgtttagcgctgcgctcgaagtgtcttatctaacggaggactaaaattacctggtcactccttagacttttcgtagtacttaattgccggacatccgttgggctacaccagcaagaacacaaagtggtatgtgtgaagctagactgacctcatgattcgtactacattataagaatcaagcttcccggatttgtgttctgagatattaccacgtacatttttaagggggttcttgacatcgtaacgctaaggctgattaaagaggagggtgctatgcagagtttattggtgtttcatcaatgtatcacacaaaattagctactataggaagtagctttggtgcgagcagggggcggtatggttaagaaagctatggtaagaaaggcccaggtgatactacgtgtaaggttgtgaagagccacaagagccaagttttgatattcgacttcctccgaatctacagcttatcgagggttaaacgttacgcatattacgagattacatgatagcttctcagttctagcacatttatgagaccctttgaatggtgtcaataaataggaggtccccatatgacaagtagaatactaactataagagatttgtaacgctggataccatttgcagaggattggcccaaagaatgattgcccaacgcttatattgtcagaccttgcattagaagaataacgcagaatacgactgcagtttgatataattttggctctgggttgccttagtatcattactaatagacttgtggtctatatccatttgtttaatggaatagactgggtaaaacacacctcttccaggctgtagttcttcatgttgtaaggatccgtcatggcgtgcaaactaggggaggtattttttgctaattgcggtaacggctccagttgggatatcgtcaatatgtgccactcggccctttctctgagacgctaagatttccgtaaggtatagcgataagagtctctaatgccagaggaattgttaccgcgagcaagattcatgtctatatataaaatatcatccactttgaattactggttggaatcatcgttcgcgttataacaaaaaaccttttaattatgttaccacagatctcgaagtcccttttgaggcagaagtttaaatataagctctaattgtcgcatctaacgggtatatcgtctcaacggtaggtcaaaaacatttgttaacttcagactgtacattcgcatttaactcgccatgtaaaccgcaatacatctcgtgcctatctctcctagtaacgtattatcgctgggtgaaagcgcaactaagtaataagtgaatgtcattcacaatacctaactctatccgacgcgtaagagcgacccagcagtttaatgacatgataaatcaaattctatgcaaggcagtacttgctttgtggacgatagcgattttccaccgtattgcgaagtcagttatgctgaaattttattccattcgcataacaccaaggcttactcttaggaaaaaatgtaataccgattttggtatgaagtatgttacagtacagaatgaaatgcccggcggcgtggtcaaactgtttcctgaggttcatatagggaaaggtcatccctcagaattggccccgtaatcgcaaagcctacgggagctttcttaagtccaaccggtaaagccaaatctcaattcatatgaggaaatgtttgaccgataaagaatagattgtcgaactaacagtcacagagaaaatacgagtagcatcacctaaacaaagcaggtaataaaatagactaatggagatcatcgtatcggcttatgacctgcgtccatttaaaggcaatgaatacattaccgactaga

SEQ ID NO: 28 - DNA, 2000 bp, Artificial Sequence
  Synthetic
agttatgaggttcacttctcatataacactatcaacaatgatcatctcttgcgaaacaagcgccctacacagcttcaatggaaccaagagccataatgaggtaagggacggctagttactaataaaggaatcgattttacaaacactaaatgaaaaacttgcgctggttgcaatgctataaaaaaatgaaatgcaaaccagtgaagatcccgatcaaccgttcgctgatttttattgatgctgtacgttgtgttagtttaatgatatataggccatctccaggttacttaggacgccaaaattactattttgaagctcaaccgtggtataatagctacaataattaattgatgcctgcaggtcgtatctcgaacgattgtacgcattacctatgatatgaacagaatctgtatcccatacttaaaatcttgaccttgtaaagatttcgcatacgcattaagaaatttcgttctacccgcacggattgtccaagtatatctggccattcacagaagttactaatcttcatctctaagtttaaggccgacaaagggtccaaaacctgcgtaggttacaacgcagcttacactcagtgactaaccaacgctcagtagggtaactggacttgttctcgctattcagctggtactgtaatgatcaacttagaacggccctatggctaagcaaggagtacgcaatgttttagaatacgtgtttgctcacacaggtagtagtttaatataccccctgacaagatatgttaacatagatgaagtttggtattacttatagccagactattcttcaacatatacactgggttttaggagtgtgcaatttataaggacagttatattcctacaatcgttgtatgatccttttgggtttggtagaactacgtttgggccgcgcctttggtcaaccacggactttctgtctagatgccaattcctacaagcttagtcctatcaatttagtagagaacaaattttgtcatcactgaattgtcgtcttactatcggatcattctccgctaattataggattattagtaacgcgtatataggagcgattaatgactcatcaatgaatagcatcactaggtgtattatatgaacctctctctattctattaactgcccactgtgggtaatttgagttatacctgaccggtccctcggatccttaatcctttgatgtcgataggtaactgaagtgtaagatcctgatatatgaagccggtaaggagacggagattttatattagtgttcttggatactgtgctagaaggttctactctaactcaaacaggttataaagtaggaaggaaaaagttgatagtggtaaactaattatgagttggcttgcttattccaagttagcgaggttttcatgacgtaagtctgataaggtttgctggaagctgaaaagttttacaaaaacgttgttttagaatggtttgtccccgaaaatcgaacctggcatagccctcaggagacgaacaagcccaggcaaaccgggggtttctcgcttattgctataatcacctctagtgttgtagaagcaattacggtggggaggcgtcaatgtggcctgagttccgttgaggacttttcacgtgtaggacccattaatagaggagatatatgtctttcagctgcggaattcataatagtggaaagaagaaaagggattactagattaatattactcatcccagacttaagttgaaagctacatcttcacacccaggaaaccggaccgcctttgttcaggtctaagtagtctggaacagaaccgtatcaactgccccaattcataggtgttagcgtgacagcgatcgcggatttttagtccagactggctgggccatccgcttcaataagttagaggactacatacaacgatggacccaattggcaatagtcgtggtaaacttcgaaggggcggtgtaagattcaagctgtagtcgtgatgaaggagatcatcgtataaacag

SEQ ID NO: 29 - DNA, 2000 bp, Artificial Sequence
  Synthetic
atacatctagactactaagagggattatcccagcgcagtcccacccaaacatcaatctgtccctttgttctaatatatctctggtcgcgaatgagtaaacggggctaaaggtccattatttttatgtaggagcatgttgcttattatggcatagcagtcgccatccccctgtcactcgatctagatacatctcacattgattggaaacttctacaaaacgttagtacttaagatgagtgatttagtgcatttctcgttttcacaaactttgctaaacaaacgtattgagtggcgcgttttttgatttgtcgcataaccgtttactccctgttcgaaggaaatcgatctccttataaataatgagtacattatacagctagcataatctgcgtgtggcaaaagtgaacgtttaatctacaattgatggaaaaatagcccgttagtccttttaaagacgtcttggaaaaatattgagacaaccttcgtccaaaatatgtcaaagcttcgtcacatcttttcacctattactaactccgtagttcaactgactttagagggcaagttttgagacaatatcttagggctgactaataagacggttatatttcaagaaggaaagatcttaagagtcaaaaaaacgtcagggctatcgttacgatattggtatgaacagtaatgatatattttgcagatcttaatataacgacattcgaacacaatagcgtcagacaaaggttaccactcctctataattactgcagcttcaattgatgagcgtcatttaattttggccggacatttacatcgtgagctggcagcacgctcagctttattgttcttgccagaacattacgaatagccgttcaatgccaattagtatgataaaagtagtgagtgtaaaacatggcctgggtttaaagaatgagtaactattattttgtaggaataactgattcccttgagttctatcttaagttgtacagaatcacactcctacagcgaataagcaacgacatagaatccgttatttcgtatgtctcggcgggacatgtataagtagcatacgttatatcggttgtcgcacgaaccgccttcattccaaaggcgcttacaaatctgcagtaaaaagcttagcatttactatagagtatcggcgttgaccgttaagcccgtcccgtccattcaatcactcaattgatcatcttttggcaatagtcgtcatatgagaaaatagctctgtcgttgttattattggctagagtataagctgttaaactacagaatgacgttttgtggaaagtggacgtaagatccttgttcgcgaagactcgcacggtggggaacaattcctgggaatatttgatctacgtacggttattctgcatgtgattacaatatttccaacgcagtccttttgacattatatgaaaccagacccgatgcatatgttttctgactggtggtttgagtcagagtcaacaaaagtatcagtctttcgttactaaatcttcctaagtaaatggtgggcgaccattccttgtaacctgttctgttataggtactattccagcctggaaatcgtggaacacatcgatctagttgtctatctataagagaacactcggttccaaatatgtaatccgcacgtaagagaggagtctcgtacatgatatataacgttgggtacatttcttagacattccggtgatacataatgtacaagtcacatgattacaccagctggtagatagaatacctgagactgggtcctagatgattataacaagtgttacatggacgctctcgttttgttgttggcttaacaccagggcttgctccatgttctcatgtcgttattactgaattatcttccattatgatcctggacggatgaacgaagcagaagataacaaagatgactgaatgccggaaaaggaattaggccctgatatatcgcgcttctttatgcatgtttacgctgtaccaataaacgcaagagg

SEQ ID NO: 30 - DNA, 2000 bp, Artificial Sequence
  Synthetic
gtacccgtatatcgtcacttcatttgaagctattattaatgtaaaatccttccgtcacacactcttttcaaaaagggaagtctaaattaacattcagatgaaaagcgctgacccacatgggaatatcctttctacgctatcagccgaaaagctccagcgattagctaaatatctaagcctccagaacagagttattatatattggttcgaatatgctaatattacagtagaaagtaaggtaccggcacttttaacgccgaagtcgaccggtgtagctgtgaaaatatatttagtacacgtaatattaattggaaattgatgagatcgaatcttcaggagaatctgacgagcattactaatcgcgcgtgacgggaacgttaatatacaagcgtctattctaggttataataaactcctatctggcaagttgaatggttttttcaaaactttaacgttctggctatacaaagctagttgctttaacttatcgcatactatgatccttcccatcaatcaatctcagtgactataaacgcaagtgacacaattgtctgcgttccacatttctaaatctcttatcgctcattccctctacacaaagttcgattaccaaacgcgggtctacacacaagcttacaaggattacaatatccaattttttgttatcaaaggcgaactcaacgaatttaatcgttggtcattggtatggaatggcgattataagaaaactcttttagtcatagtagctcgagatgaagtgaaccgggccagtcggtagtttcactatcgcgcagtagtcacgatcagttcttagaatctatctcctaatcaagtccaacaagcaatccgaaatgttgctttctataaagggtatgtgtacctgccaatattaaacttgattcactcaatagtgattttaaatatgtccatatttatgcaagaatcattgacattagtaaattcagccgtgcatttgacacaataaaggtagatttagactgcatatttcccgcatatttattattgtcaacgcacaaagttgatggaccgaccacgatcgcatcgaagaccgtctaaacgacgatattcttcggagatccatatttgttttcaattaccgaccattgttcatcaagtgtagttcagtcggaaatttttcgtgtgctttttaaaataccaaatctgaggaaaaagctcgctagatgttgagtcaatccgtaagaatatgccccaggagacatatgtaagtcacagccgtagactctcggttaccccacgatatgttccatatgcaacgtttgttgagtaatatgcagttcagtcgggcgtattatcaacagacagactggcacagtaaattttatcatcgggtttaaaatatctagatacctcagtttcaagggggagttgaactttaacacgagatcaaactacatacacaagattatcagtgggtacgctgagacttatccttagcctggagagagtccagctacaggaactgctagtacttagcgtgcgacctcaaatcgagagaactaattaccctgatcgacagatcgggcaagttaagcaaacgcggctcgcgtgtagaaccataacaattggagatgctcctgcttaagagattatagaaccgcaacccatcaatcgtcagttacccgagggctcacgcacgcggtgatggaagttagttcctttgtacgcacgagctgcaatacgtggtgattataatcggcgcacactaaaggggtggatacaatagtagaagcatatacgtcgcataggcgtacgcgggcgaaaattttaatcgttaacgtggcactaacagcgttttgtctccccactcgtgggttgcggtgcatcgcacatattcccacaacacctcttaatgctttattatttgtattaatggcgcgaatctgcctgatattagtattcgcactagtgggtaacgaaatcttagtcgctggctactgcagaactaattgcgttgcgat

SEQ ID NO: 31 - DNA, 2000 bp, Artificial Sequence
  Synthetic
actagctacagatctgtaatagaaaaatgcagatgcttgttctgcgtcgactcgctcatcaacatcctgtctcacaagttatgcatcctgtgcattttattgaagctttgatggggattagatcgtgtatggaaatgtttattcgcctggataagatctgtcggcttattcgtggccaataataggtcaatttgcggaaacataaagactcgcataccaatactcgcttatcctgaggttaaatttagtgtatgtagacgaacaacagtatttagtagtatgacgttcccccgtattgccagaactcctgaatatttggatatgaggtatgactacgaaaaaaatactacgttgctcataaccattggtgcagggataccgaactcattgttaagggacgccacagtccagtctcttttcgttcagagcgtgtttttcaaagtgcttgtattagtgtggacagagtttactgatctctccgcacttggactgattgtgatcccgatcatctcttttcataattgtaacacgctttcatagtacacttctgtacattgaagagtgcttgcagccggacagtcctatagaatttggcgtttgttcggccaatgtgtgcattttaactttaggcgccatctcttgagattactcctttgaaaaattttggcggaggttaactctggtctttaacataggcgtgcttaacacgagctttacggtcaggtacaggtaacaaaacaggtctaaatttatttaagcagcttctgatactttccaagggtcacagttggggagccttccgaggtatgacaatcagttttcaaaaggtgtagaatatcatatattctatctaggccagagcattctaagctgttaaaagagtgctatgctcagaagttgactgttctaatcgaaaatcggacatagataacccgcataccacaagtcccgttgtaacgtacccatcgtttttgattctatgtctttgctaatgattggcgattgagacatcctacttctgtagcttggctgttatgcgatccaaaatggtatccagtggtggatgtccgccgcaaactgaaactccctatcagttctttgaaattaatttgcgggctatccgactcattctttaggaattaacagaagaacacgcgtctgtaccaaggttcttctttgttatatcacataacaatgaatcacgttctatgatgaatccaggtatagaagttgtaggtaagcacttgtataagggggcgctcctctcagattgattcattatttactaaaaaaggagcgtgttattacttctaacaactcctcgccattatatattatttaactaccattcccactagaaatggatatcgtgttctaagaccctaattgtgctcattaaactaactaccgcaccaaccgccttgaatcaccggaccacactagttaagctgccgatacccaatatggtattttagtgtataccggatatgaccttatttacgaatggattgagctcaccccatagatcagtaccagcgttattatgaaaatcttgttattttaacagagagacatgcttggtcattactacgaatttgagtttacgttatacaaggcgatccaaacggacaatagcgcgatacgagattatagtaccaatagcacgaatcagttttagcgatctcgtccgatctgtcaagccgaatgactctgaaacgttagtatctgaaacgtttcattcagcctaagatatgtatagtatcattataccgtgtgggtagaacaatcaaatgcagataaagctatttaatgcacttcacataacctctccgttggaaatccatgtattctctaatcaattgaattgtaccttagaaagcacagggggacacctgaagacctcccatctcttaaggttaccggcacgtgaaacttcaaaagtcagacaatcaaacggcaacgtgaatgtcttcggaagtggtggtatgcacatcgcgtca

SEQ ID NO: 32 - DNA, 2000 bp, Artificial Sequence
  Synthetic
ttaatagaagtaataagtgctattggactaaaatcgcgtcaattagctatagaacagctctgtgacgaactatcaatggggcattcgttcactagtggataccgtacaagctcgccgtgatcgtgcgtcaaggatagtgccagagcgccgcgctatatgtgtaacgacgcataagtagatgtttatgttattgggcaaagtcattcttatccataataagcgctgccgataaagattcatcagagatattgagattctccatacttgactaatctctgagtaattaaaatatatttctaatcggataagttagggatcaccgaacccaatgaacttagtttaatgtgttctcgcgaatatccccatgatataaagatccgaatacctcagctccgtgcgtgctcgtgcagtcgtgcgttttctatgaatcaaccatcagtaacgagtagcggtaactacttctcgagtttaaccaaagcctatgtatactagcgtgcaatcacgtgcggaaggtccgacctacagcagcattttcgttcgaaaaacgaaaactaatgtgcactatgttgaatgggcattcaggccttaacttctaacgttaaactagatttgcgattattaggtatgagatcgaccaggtcgccacagataattaaagatagccctagcaaagtgataaggtccggatgttagaacttgcaagagtgtgtaagattatttactctcggtgcgtcgacaggcgaaacccataacttttatcggtcaagattacgaccttcagctagtatcttgagatttgaaagggcctaaaagcaatttagtgtacttgtgtaacataaccttaattattgatggttctatcgactcccagcggtaataatcttgtaatattgtcggatttagttgaagggcaggttgacataccgaacaatagctagtatcaatgtataactagcaggcatctaatttcgtaaacactcctgacacttgtcgtgtctaagcatgttaggacaaaagaccagtttttttaaacctgactgtaccggcaacgccacagattttatgtctcgcatacgtacgaactgaatttgagggggctcaggtttggacttacaccgcacgtgactatactgagatcgaggctccattaacggcaacataagactagcactgtatgatctgaagccaggctctggtgaaattgcgggtagttaacgacatttatcgacgaacccttgataaaaagtgattatgttgtatctgcgtgatatattcttttcgtgttcagtctctagaacttcgtgcgtaataaagattatagaggaacggttaacctcattacaagacggagaccgttcatagacgccgatggattacagggtctactatagctacctagaacactggtgaacatagggataacatacaattaacaatattccgagccaaattatgtcttgagtcttggttgttatctatatcgttattatgttagaaactaataaatgcgataagaactagattttacagtagatccaaataccggaatctatcgggacgattgattaagacttactcaaacctaactttagcccgattttgcaattagagatacgtcgatttcgagacaagagtagcgtccccatggcaaatatccacggacagataatgacacgtgagggatggcaagagtagttgctcaggatgtaggcgttgatggtctggcgctaatgtcgtggctacctgttgagtctcgcgtaatgactagtagtgttcgaacgtatgaccaagttccttcctagtgttaccactttgacacatacccaggggtttgccgcatgtcgctactatagtataggtgctgctatgaagcttctgaatcagcggctaacaagtacctaagaaaattggacatcttttggatgacagtgcacaggagcctatactgaattatcggtgatcgatgcttcatgtaatcaaaaccagcgcgtacacacttt

SEQ ID NO: 33 - DNA, 2000 bp, Artificial Sequence
  Synthetic
tactcttaattcattacatattgtgcggtcgaattcagggagccgataatgcggttacaataattcctatacttaaatatacaaagatttaaaatttcaaaaaatggttaccagcatcgttagtgcgtatacatcaagaggcacgtgccccggagacagcaagtaagctctttaaacatgctttgacatacgatttttaataaaacatgagcatttgaataaaaacgacttcctcatactgtaaacatcacgcatgcacattagacaataatccagtaacgaaacggcttcagtcgtaatcgcccatatagttggctacagaatgttggatagagaacttaagtacgctaaggcggcgtattttcttaatatttaggggtattgccgcagtcattacagataaccgcctatgcggccatgccaggattatagataactttttaacattagccgcagaggtgggactagcacgtaatatcagcacataacgtgtcagtcagcatattacggaataatcctatcgttatcagatctcccctgtcatatcacaacatgtttcgatgttccaaaaccgggaacattttggatcggttaaatgattgtacatcatttgttgcagaccttaggaacatccatcatccgccgcccttcatctctcaaagttatcgcttgtaaatgtatcacaactagtatggtgtaaaatatagtacccgatagactcgatttaggctgtgaggttagtaactctaacttgtgctttcgacacagatcctcgtttcatgcaaatttaattttgctggctagatatatcaatcgttcgattattcagagttttggtgaggagccccctcagatgggagcattttcactactttaaagaataacgtatttttcgccctgtcccttagtgacttaaaaagaatgggggctagtgcttagagctggtagggctttttggttctatctgttaagcgaataagctgtcacctaagcaaattaatgctttcattgtaccccggaactttaaatctatgaacaatcgcaacaaattgtccaaaggcaacaatacgacacagttagaggccatcggcgcaggtacactctatccacgcctatcagaatgtcacctggttaatggtcaatttaggtggctggaggcacatgtgaagcaatatggtctagggaaagatatcggtttacttagattttatagttccggatccaacttaaataatataggtattaaagagcagtatcaagagggtttcttcccaaggaatcttgcgattttcatacacagctttaacaaatttcactagacgcaccttcattttgtcgtctcgttgtatatgagtccggggtaagaattttttaccgtatttaacatgatcaacgggtactaaagcaatgtcatttctaaacacagtaggtaaaggacacgtcatcttattttaaagaatgtcagaaatcagggagactagatcgatattacgtgttttttgagtcaaagacggccgtaaaataatcaagcagtctttctacctgtacttgtcgctacctagaatctttaatttatccatgtcaaggaggatgcccatctgaaacaatacctgttgctagatcgtctaacaacggcatcttgtcgtccatgcggggttgttcttgtacgtatcagcgtcggttatatgtaaaaataatgttttactactatgccatctgtcccgtattcttaagcatgactaatattaaaagccgcctatatatcgagaacgactaccattggaatttaaaattgcttccaagctatgatgatgtgacctctcacattgtggtagtataaactatggttagccacgactcgttcggacaagtagtaatatctgttggtaatagtcgggttaccgcgaaatatttgaaattgatattaagaagcaatgatttgtacataagtatacctgtaatgaattcctgcgttagcagcttagtatccattattagag

SEQ ID NO: 34 - DNA, 2000 bp, Artificial Sequence
  Synthetic
ggccctatagattttaacctaagctctagcttgtgtgtgctcagagtactgctcataaatatgctcgataaaggaggtaaggcatatcgtaatttggaagataataccacacttattggtaacacgttggaatcacatattaattatgagccagccttggcattcgagcagggatatgtgggagtatcagttgagtttggctccttgctactgccctctgatgctctgcttgctctagcttaggtcattaatgataaaaaagagccagagtgtgggctaaacaggcaacggtaccgttgtagagcgaggtattgctatcgggagacgtcgggtcaaagtgggattcatgcagtaagtttgccaaagggtctgcttaaagagaccgattccggaaggctatatgccatagcaaggtatgcactgcattgagctgaaaactcttgagcatagtatttactaaataaagaatctgatatcttctagcgtgttcactggactattatttagatggtcgccaacaacaagcgtgcgaatcatatagacccaacccagggtggtattgaattctatattaaaatgtctcgcccttataactctctaggtttccatagtacaaacctaggtgtcgtcaactgcatgcactgctttttgtatcggtaatgttgatcgacccgatgggctttttttaataaaggtcttgtttagttgatcatactaccaattttggtggtcgatggctcaatgaccaatggaatctttatagtaaaagagcccttggcaccaacgaatcatggaatttaggacgatgtctcatttaccatattttgcattcagactatgactttcaataatagaatatcatcgtcaaacaccgtggatatggcatcgacaagtgttgggatgcccactgaataacgtctcttcgtcatctttagggcggctatccattaaggaggattttatttttatagcagtcttagtccgaggcattggcgccaaacatcggctcaacactagacacgtctttaatggaaagtatctagtgttactgcggtacggaaagcaagttcagtacttttatccaatctaagtatcacccagcttatatttaaaagctaggtaatagggaagttactaataactcatgcgcgtgtagtgtagtcttgctgtcgcttaaagcaactgaatgaatgtacggctgacaaaggcttacccaagaaaactctcttgtacgctacaagaaacctgtaacaagagaaaaatattttagcccacgtatagtgaggccaaacttgatgcccgtaaaagcaaacaagtaatattcagcagaatttgcggtcattcaagtgtttaggtacgtaacttttacagaattagctgttgattaggtaatactaaatcaaaatgtcgtaataccgaagcagaagtatatgatctaatttgtcgcctcgcttcatgctacgaatgttacttcgtttattacagctgcaaacttgcagtgacttgcatttgataggattcttcctagggaaccatactgggccgcggacagggagtcaggaactcataacggatgaagatgtaatctctataggggtgaataacaggattgaagatagtaatctaagtactctcatctcgtggacgactttaagcgcactgacagcgactcgcgattcgacgaacacccgtgatcgatttacacgttcattctgaaagatatacaggtaataattctaaaagataattgagtaccaatatataggttttatgatcttaggcgcatgtcactgacgagagaaaagatagtcttgccgcctctaagtgttctatttctggacgtgcctgggcattaagggcgacgttgacttttatacacatttcatgtccactaacaattttatatcacgtagcaggacataaagggaggactctataaaaagtttcgctatatacgtacagtacgttcaaaatctccagaggaaagcttgtaaaaaaag

SEQ ID NO: 35 - DNA, 2000 bp, Artificial Sequence
  Synthetic
cgctcgacacgagtataacaaatatcgatagatgctatagtgataaggtataagtaaaatagtactgcgaatacaaatagcttggagaaatacgttcatcctttaacttcaaaaatttttggacctcaggcacgttgtcattattactggcaggtgataccacccaaaaatcgtacccgcaatatatcttcggtaattcttgccaagttgggattttacatacttagtattaatagtgggatcagcttcgatcgaagaccataactcagtatgtgtattcctcatacaagatttctgaaggacgaaggctcatcaatgctgaggtgttatcaggtcaataacaagccgcattaacgccgtaaccctaatgccataattctttgacgaaatgccaaatagtttcatcaggaatcacattatttggataaggaagcacaacaaacgctttaatctatacccctagaattaagaggacagcatgataggctttgcaatgaaccagtctcctaagcgtaccaccactccggagccttatggcgcgccggtattatggcgatgcactgcctgggcgaaactcgagtgaatcatttttcccgatatacacagcagtacgccgacggtctggtaaaaaaaacgttataggctttgaccgcatggtgatcgtggttaagtgcctttacctagagtgctgctagatgtaacacaattgatctgacagtttacgaccttgtaatccaagaaccatatagatgacccgctgagttagtaagataatgcacgctccggggctaaatctagtgcggttcatgaataccgaatcaactacggttattggctgcggtagaatatttagttgtgttaaatatactctaagatgaacatgtatcactataatcactcaccccctctgcgttcataagtaagtggctagtgtgatagtaacttgtatcagcgaccactactatatgtggaagcttttgaatgagaatctccgcacatgatgatgtattgatacaattcttttgttcgaaaaagcttcggtgttttttaggacaggagattaacgctttagagtcatacatatatgtcaagaaaccggggaaaaaatgccagcccagagtgttctaaacgataggttgttcagtttttaataacccgcgacgcgtcaagtaacgtcacgggtcagctacgattaccaatttgctataaactttcccccgacgagccaaatccctcaaagctgccagataaaaggatagcaacctgtactccccgtcaaatctaatgcattcttgttttttaagtctcgtgtaacatgcgttggctaatcttctctaccgggtccagtgccctttcagcttatgcctcacctttgattagtaatggacatcagcttttagtcacatcggagtgccaattataccgttatatctttctctgatgcagaccgacctgtcgtgtaccgattcatcctagggtaactagccgtggcaaaatatctttatcgtgttgtcaggacttggttgttatatactctagcccgtagatttaaaataaattaagtgtagatcgtccaaatatctaaagcaatcgcagtttttatcacatcatgtgttaaaatgcgatcaaaagaaaaatactgttatttcgagagtcaaggctgtgaggaaatatgatgaagactgccatcctggtggactggcggccccaacgttgaagtttctatttgatcggttattaaaggatactcgagaacaacatcgaaggaataaacttttatagaaagtctccgaaatgaataacttaagatataaatttatcgcgcgatagttctggtggatgatagctttattcctcttaatgcagtatagctattgcacctattaatttgtataataacgtatcatgttagacggtcagcatgatattccggatagtggaagcaaattacgacatctaaatatgtcgctagtatttgagtcattatagcttcgaggctt

SEQ ID NO: 36 - DNA, 2000 bp, Artificial Sequence
  Synthetic
ctctaacgtgcatttcttcgtcgcctttgtaagaccccacaaaaacatgacgctttagggatatggtccaagactccgaattgaaagtatgctggtatgatatgggacgtttttgaaacccccctctcacgcgggtaattgggtttttagttagtgtatcatagtaggtatatctacgaactacgtctgactgagagagactttgtgcctctcaaccgctatggtgtcagcgactgatattggagttatttacccgtcgttatacgtgggtaatctttactacggttcaaggtaactaatctagtgtaggtagaatgctgaagaattacccgttggacccggtagtccgtccgctccacgcatggaatgcatgagtaacgtctaggtgaatatccggagtgcataactttttggtatctagtccgctactggatgcagaatgacatatttttttcgagtgcttactattactcttctcaaacagaacgatcattatgttgcttaaattcacgctatgttctcgatgtaaaacaattttcgtagagaaagatgcgtaaaacgcagagttagcatataaaaagtacaatcaagcccgaagcactcacaagaaacataggggctaaatgttaccgtccaagtgagtaggatttaatatcaagccgggcttattgggtacagtacgtggacggactacgacgcatgtgtgttatagaatgaagtgcctacaactgaagcacaattactaaaggaatgtacctgggtttacactaagcatcccatcctcttcgcggttcagcctgatgtaaacgtaaatctcgtcttcccattattaagacgcctcgatctacgataggtgatacgtgtacatcggtggaccatgtgttttgatattcaacgatgtaagtatggttccctgcagtgaacccctcttcaagtcgtcgatgtacctgcaagtgtacaatcggaagaccatgggtccatatgtaaaaataagttaggggtcttttggtctgtgttggttataatcgatattgccaaaatattatggacagttagttcgaattttgtgtatggtagccgtcgaaaagggtggacgttaagtatatccatcccagcggctgggagatatgtagaccgacgagtgttaagttattccacttactttaggacgaaatcaatacgattattttacatcggaggacatgacaacaaaaaactactcggtttcgacaggtggaagatgtcgctgcgcaccagtagagcttaggagagcgacggtactcatttgcagcatgggtacgtaatcacgttagtaaataagtaagtatgccttctcttatgtcattttataagctataatggtgttgtgccaacttaaagattgacacatgatatgctaccagataagcctcgagtcgcctatattttgctactaaacctgattaactagagaataggtataatccctggtaaccagtaattttaatactatgttgccacttgatgtagacctggctgtggttactaaggtgctttgaaaccattgaccacccgtttctgctcgggttgtgcatctaacgtaaatattcagagataacgtggctctgctattatttttatattgcctgctgacatatcatcatccttgaatggccagcaacagttcttgatcggcagaggccccatgaactagggtaatatagcagattaactatcggttaactgtattaaacttgtgtaatacttatattgactaattgggattgcctttgtcgttatctcgtttatcttgaaaacggtgatgtttttagaggcgatagtattgaatagctcgaatgatcaccagccatcaagaatgtagctaactccgaaactccttgacgagagctcaagcgaatactaggtcggcgctgctatccgcagagttcagggttctacccggggtataaaatcccattgatcattcagatattatggacttggcgtttatgcgacgagtc

SEQ ID NO: 37 - DNA, 2000 bp, Artificial Sequence
  Synthetic
aagaagcagctagtgctacttcggaatagttgtcgtttaagtccgttcaaacatgacgctctagtcattttgaaacctaaaccagtaataatagactgactcagaatgattatactgctatctctagtttaaggagatccagcgaaataacttggtgaactatgccgagatactataaaaagatcaaggacgggtcgctcacggttttggtttattttactacttcttcgtggctgtattagtcgatgcaagttctaataaatagcaaacgttttaagtgggattagtacatattgatggacgtccaccacgtcaaatctcgcagcgtcatagaaggagctataaccattcactgcgactacgacatgtgtttgggtagtgccaactacccgcttccgcgtccctgccgttctgtacacttataaaattgatattttaatcagtggatgtgctgatacggggcactgagatgatgaatagtattaggctgtagtaccttatgtacgcaagaaattttagagtaaagattagtctgtgggtaaggaaaaagctaagttatgattatccatggccatggcatctacaagctgatgaacgtaccaacattatctaatttaagaacttaacttgtcttatcctctcttaaagtcttaatttgcactattaagcttagggaagtcgcaaccaaactcgtgtagtattgagataaattattaaactttcttagtatctactgatatccgtatcaagtatgcttataaattcttgttctgcctgacaggctagtgaatcctgcacccgggacgattgcaggtgtatacaggccctcacgctagcaatcaataccaatacgaaataagggctaacatttttcgtaacagattagaagcagtcccgttcagaacttaccactgcaccaacggaggtactgaattcggactcatagaatcctcgagtagtaagaccgtagaagagacagtgcatattaatgtcatagatcaatttatattttatatggttgcccatttcatgatacccctttaaatttataacttagaaaaggagccgcactaataatgagcggcatgctgtaaaaaagtaggccaaaacgcaagataaggtacctttgttgtccaatcaaattaattgatttattcttcgatcgatcgaccgtcatagttgaagtaactatttagttacggcagatacagcgtatcaattcattcggtgactttgcttagataactgctcgataatccggaattatcatcgttcaaagtccttcccttactaaggctcttggattcagatgatcggtcatccctaacaaacagcccactgccatgctgctatggtgacattcgttactacattgatttctgcagaccttcatccataatacgatggtaacgtctcgcttactatgcacggtgtgcccctgcctatatcttcacgatataccaagtggagaaccgtaggcatgtagtcattcaggtggccactctccttcacattatgtttagaggtcatgaataaccctaatcgtgtgacctcaaacagcatcgtattccgaataagtaacaagtaggggtgtttcaagttgcatgacacaataggatatgattctcaaccaaacttggcaataaacgcataggtttagcagtactaacaagccattatgtttaatatagagcatggcttactctgtcatgttcaaggtggctaaacccaacgcgttaatacactcatcggttacagtgtttttagaagagcaattgatatctcttcaggtgatacctggttcattatcctaattcagttggttcaggaagccttataactaccaattcgatatttttaagcatatagattaggtgataccacaccgtaggaaattgtgcagaatttggtgtctagaaatttaacattaagtgatcagaaaattctctgtgttaaacgactgttgcgaatctgtgtctttcaacctcaagtacgatctc

SEQ ID NO: 38 - DNA, 2000 bp, Artificial Sequence
  Synthetic
taacaacctgtaactgtcaactaatgacctccttaccaaaattgagggtagttggttcaaagagaatgcagcatgacgcagagcttgtagtcacatcgttcttctagtacgcagagtgtagagttaagattattaaactcagagcacgttgtggacaaaccaataccagtccattcaattacatggtatctaacagtatcgtacaactttaatatggtctagggctagtgaagtgtaccaactacttgatacgcagtaaataatttcatcctatctttacgtcgccatcgaaaagcaaagttatggcgcgtggaaattcagatgaaccataaccaaacagataaattggcagcagttttttgtagacatttatataagaagagctcgaggcgtaggttaattctatacaacgctatgatagtcaagttctacttgaccaactacgctgggaatgtttattaaattcaactgggggcaaactagcatatactgtctgagtgtccttcgatggttctatacaaacggggtgtcgaggtactagtggaatggagaaactaccgacaaacgcatatcttatcttctactcgggatttatgaaattttttgcgtatactattcctgtgagcaatgttcaacagcgtagtgagcctcataacgtcacatcaattgtttcacgtctgtggctatcgagtattccttaacttaactagagtatagacattagagtctaattctatgcaagttagataactactactactgtcgtacttcattcagttcctgctcgtactcggcgacgctataaccggcctagtttgtgcgtcgccagataactgttccttttaaacgtataaaaagtacgaaagattaacccagcggaagttgggccccataaatgtcatatagggactcagactactgttaaaaactcctagtatacattgtagataatcaactaaagttggactatcaagaatcaaactgtaatcaggtcacagaacaaatggactaatagagctatctaatcatcatacagatttatacccagtggaaacaaaactttaccccttgaggatttactggagttgtgtcaagttagaaatcggtcaacataaattagaaaatgccttggaacgctgtataactgatcacatatagctgtgcctaatgcttcaatcgtcaatgctgaccacaatctacctgacttggaaatccgctacacccatatccatatacttaaagaatccgtactttatatcctattcaccgatgtccgatgtggcgctatgtgtgtctagtagtatatcagttcaaggcgagaatgaagaagaatacagggtctctttagagcactgtgtcactgtttcttaggccagttaattctagaaatcaaataaatgaataactcgcgacggctcaaaagaaatctatggtttacgcataagctgtaggtacttctaagcttgatttgcttccgggggatcctaatctaaatgtgaaggggcagatttagatctctgctcattgagtgggaggttggacattgaacatagaactaccttccctgcgtgctgtaagattatgagaatctatgctcggtcgttgtctaaaaatcagactacaagggtaagaataataacagaccgaaatagatgtctccttcaagatagtcagtttgcgcaagtctggcaggaacgttaagtaatcctgagttataatagcgcccttttaagctttcctggcgaaaaccgaaccaagcccccgtaacacaatgtcactatccgtacgaaagttagtgtaataacgactgtacctattataagcacatttggttggctatcttctccctagattcctggcggaaaagaagcatgtctacgttcgataggactcatttttgaggaaaactattataacggctataacgcgcgattaatccctgtcggtccatcattcacgtgagtgtaaaattgtgattagtacttaaacgggttcgtgga

SEQ ID NO: 39 - DNA, 2000 bp, Artificial Sequence
  Synthetic
ttgacgatttatatagctactacttagccttactacatattccggcgtgccggtagatatgactaagttaatacttacagacattcaatattaggatttcggtgacctcgatctctcttgattgaataaaaaatggatattaatgcgtcgatagttgtgataagttatgtatgatgtcctgagggacatatgataatcttctaatagttaccttaaaccgaattgtgtttatgatgaaaaatataggtgaagttagcacctatcaccagactttgggatagttagtccgtaccaagcagcagttcaactgacaggaacgtcaattctgtctctcattactttggccatggattgaaaatcgacttcagtctgactcacaacagttatagaaggattttggctcaccactcttcgaaataggtcatttaatgcgtactgctttttttgacggccctttattcattctattgagggaatccctaactttagccacacgcaaactggtttatatggatactctcaagattgtttacatatccagaagcttatacttcctcaatgtgatgcacacaaggtgggatcatcttgtttctacaatgcagaatgaattaaaaatcgcccttcctggcacatcttgctgtacggctacagagtaaaattagctcgttatttatgagtgtttacacaacccaaatctaagtcgaatgtactttaaacttggcgtggattcatagacatgcaatcagtgttaaattgtcactcaaacacgtgcctgacttcagacaaattcatggattcaagctgctaatattcacaatagacgagataggggcgtagctttttctgtacgatgggggaatatacgagcatttctatgaaccaaaacaggcaaaatgagcaaataccttgtgcatcatatagtttccatcaactggagaaagcctcttgatcggctacaacttttcaagtccttgcggcgttggccctgaagtactatagccttttgttctcactaatctagccaatcacttgttgactattcttgcctcacccatagagtggtaatggaattccaaaaacctattcccgagtttaacccgtattgtttgagaggagttcctagtgtcttcattaaattgcacatggactctacggaaattactttttattaaatcatagaatctctgtcatcagtccatgcgtcctcagtcaataacggtcgccgtgtctacggaaaggttcattctatgcctgtaaagtacatctaacacaatttagtgtgggtcttctactacagttcacccgggaaacgttttatgtacgagtgttggtaaagcgtcctcatcaagtcgatccattgtaaggaatcgactatatactccagcttaactaggaccccgttacatcttaatggtaggtctaagaggtgataagactggaacctacatcatgagttgagtgagcaatgagagccagcaaatggtgggaagactagaccaacacaggatctcatgcttcctgtagcagtgcaactcagttcgctgcgaaaataattaacatatcccctattggcaaaaccctgcatacgtatttagcaaatatctgtaggggtcgtccaatagcagtgccgttttataaattgggttgatacataacactgaatcaagtgaaatcgaacggtggtaaaatggcttgaaaggggaagttgtttaacattcgctagcgacacatgttgcatggttagggttgctatttcgcctcattctcgttacgacattctcaaccagtagcccaccaacccaattaaggtcacgcacgaacctatcatccacttacctcttacaacataaaatagtcaatacaccttcctcaattagccttaatcaaataaagctagttatttttgtctcctggggatcagggcgcttacttcgtactcgcttcccccgctaggaaggccactggttcccgaagaaacgtgaataattgcacatgcttta

SEQ ID NO: 40 - DNA, 2000 bp, Artificial Sequence
  Synthetic
agtatggaaggtgcctcggtaattacggaaagagcttatctgccggaaacttttattttgtttcatcaaaaggttatacgataataccgcatctaccttttcgtatcaaaattggtccacaaatccaacttattgtcatcttgaatcacacattcatctttccgtctaatgaaggagcgtcattacttgttgtatgaaacgcaaattctctacactagtaagtgagacattaactacagcctattaaataattcaggtagactgatgagtaatatttcttctatatatatgtgatactcactctctactgagttgactagtggactctttgttcttgtacacacacaacagagaaatgcctagaacaaagtcaaagaaagcgcctagatgactttgtaaattgcaccagatctgaagtcgagtcgtgaatagaactttgcataagactctaggacttccgatggcgtattatacttaggaaaccaagccggtagtaagaatcgaggataatactctgggaagtcttccgtatttgcgtcaacaaccagcttctggatcaagcatttcttaactagattaagcttcctctttcgttttaaagcgttttacttcagcaattgtaatccctacatttgtattagccgaatagaacgatgctcctacaacaccaggccgacctcatgttacgatggccgagaccataactcttcgatgaatcattagtggaagagttatctactgacggcatgatcctgggacatgaaattggaaagcatttgcacacgttaattcgccttttacttcaacgctcggacccggtataagataaaattagaccgttatcttcgtagatcgtaatacgtatcatctcgtatatgccgcttgtattcaacggtttcctttttagactggagcgatctacgctggcttggtttaaggactatgctagggtttgtacgtaatccctttaataattaacgaccgagctgacaaactgaataagtacagcatcaacaggacggttcgattgacagctggaaacctattaggcatcttggcccttagcataagtcccagtattatttgttcctccagtaaaaatctccccggaattagagcagcggtgaaatttatggacttgacctttttggtttagtcgtagagggacaaatatcatctcatctgaacgctcatcaccagttagttcatccaaattcaattaggaggcgtcatattgtcgggcgtctgtaacggagccagatctagaagttcattgctataaagaattagtgtgcttggcacatcacctaatcaaattttgggaagcagcatagctattcaggtgttggtcaaccagataaagtctatgaagaaaaaaacctgtgttagttctgcgtattagtattgtagtataatgtacgacatcccgaaagttaaattcaggtcgcagagtccctagtccaccgttctaactcacaaatcgatgttcggacatagctatttaacagtccatatttaccttaagtgtttcgacttatgtatgctagttaggtgtgtggctcgccttcccactgttagaccacatctagacggacatcgttaataatatctgatatacacaaaaacgtttaccatagaaaacactatattcatggacactttatcatattcctcgcccatcctcacgacccagataatagggagttgtagtttttctaaacggttttaatatgcaggtccataaagcatgcagtacattactgtttaaaactttaattcagatatatcctggagaagaaaatctcgattggttaatcacttcattgttaaattcgatttcgctatacgtttctgtactaggaaatttttcatattaggcacgcggtgttggttccgtaacactattaatttcctcccggttcgatcatggcttgcggtaagtcctcaatttaacataattgagataccgaaatcaacccagcgtcgcagtattttgagtt

SEQ ID NO: 41 - DNA, 2000 bp, Artificial Sequence
  Synthetic
ggttaaatgcatgctcacgcctcgccaggttgttaaaccatgtacttactcaatttgacaactgatgtccactctccacctcgcgcgatgctactttcttaatactaacgccaccttgtcaaacacctagatcgttctaagtgtagcaccagacagagtagacaccgtaaaaggtgaaaaggggattaatttctcctccttttgcacaaaaaagttaaggggtaggccggaggaaggttaacgcgaagcacctgcgtaatcggtttcgtgctatatcggagatataccgtaatgactcgtcgacgaaagtcgaaggctttaagctccatgccccatgttggtgcgttaggactttggtaaagtggtaaaatttagatctctttgtgtcctttatatcaagttagtgtgaatgctgagttttctcattttttaatgtaagtgattaatatgaagatgtgtagtctaatttggagaaccaacttaaaacggaataggatcggtgtatcaatgcatatgaaaccggtaaatttagtcctgttgacctgaaactgatgggaacaaaccctcaaacgctatcgcaacaccgtcctaggttccatgcactattaacctgttattgcccgtgcggagatctggtttttattgttttatactctagatatattagcggttatgtttttctgttaatttaagatgcatagtctactttgacctccggcaacgtgatttgtagaaaatatttcccacacacactatatgtgctactcaggttacccatagtttatgtaataagtatcactttaaaccctccacccgcccatacaatagaagcccttcaattatacgaggaggtattgacctgactagtttaccaaagccaaagatacctggacaagttggacaaatactaaaggactactgtagcatagtgtttgcgggccagtatacgcttatttaaacgatactactgataagaaacactggggtcaacgtgctttcatcacctgtccattactccaacagtcccaattttttaaagaaggaattttcgggacagtgaacgcggaatcgctaataatattcagatagatagctcgacacaatataactaatcagacaaaaactattcaaaactttctcctaggtagtgcgcggctcttttacgtggggtttattcacctgcgaattatcctgatgcccaggagcaaactcattataataccaccaggtgacagcctacaagtttcatggcatggctgcaacctgcacacgaacgcttatgcagcatgtgctcttgagttataccagctacttgattcgatatatggtttttgtgaagaatttgataccattgacacgggatgttgcaaatatttaataagtccatgcatactaataccaacgccagagatagattgtcagtagaactcttgaagtcaatatggaccgagtgacttgggtggtttatcccactgttagaaagttatcgtaaaataaattcttggtcaaatctaatccttataaacactctgttattactctgcttcgaatatgttgttattgaccatgctgataactacatcctttatgttaattcaaggcattctctgaaagtcaacaattaacttcatatcagacatttgacctattcctcacttttctataacatgacaatcacggtgattaaaaacatgacgcgtatcggcagcaaaccactgtactgatatgtaagagcgcccgtcgcatagatattttagactctgtccaaatcactctacgccaacttgaggtcagaatgcataccgtggtaagctgaatagttcttatacactttctaatttacccagatgacgattttttgttatatgaatgacgatcttggcattatactgccaagactgcaatcaaatcctaaattcataatttagtaagtcaatagcagatctgaatcccataaatgaattctatcgaagtacctacactatgtcacgtagaacaag

SEQ ID NO: 42 - DNA, 2000 bp, Artificial Sequence
  Synthetic
ctaggtaaattcttaggtagccgagttgaactattaataagtctcgtctgtgagtatgtcttccgttaggtattttcatatagcttcatgtgcctgtaaagacagaagtataattggatacatcagactttttatcccttttacagtctagaaagacctacttgaaacatgtttcttaatgggtaacgtagtgaattatgctcgtttttcctttggtagaatgatatttatctccatatgctctgagttggataatttgtaaagaattatacacgttaattcaacctctttatcaatgaactacgcgggcttgatcagagtaaactcacaatagtatcttgatcttcacaatctgatggatattgatgcgagttatacgacctgtggcatatcaacaatgaagtgaagtgtctgtccttatgattcgaaacaaaataagtgtccttgctagctacacccacaccgcggtgtgcatcccataaaggctcaggtatagtcttgtcataagcgctacactgccattcgtttagaatcattgtttagcaatctcaaaagtaataacatccgactttcgaataggttcagtttcctgatctactggagcctatatatatgcacagacgaatctcgtacatggcataagcaagtcatgagaagaggctgtaccacgtaaatataagcctctgattacgctgaagcttaataatcatcacccatctacgaatccgattgagggcataggctttcatgtctttttcgctgtaggtctatgcgattgtgagactattgagttttccacaatatggtggtaggtactgagtagggtacatttcactgtcctattgcgctgtcgtatgtctatccgccgttgccgtcgtcgatgttataccatttgactaacagtgttatgagtcactcccttggatgcgatgtaccttctgttgtgagggatgtaagttgcagttaagcactattagcgaataacgctaggattctggaagaagaaaacacagggtcgcttcaggtctcgagaatcttacggttagaaaatttggatctgaataaagagatgtctagccagtgtgggggttgaataagctaaatgtctgcaatgtgtatgcttctgcacagatattaacaaatccgccatatttaggcacatttggtaatggctgacaatcggatctcaagaattctatactgagttatcggactacaactaaaaagatgctatataaaattgtcataattcatgaaaagccagtaggccgaccatcatcgctctaagttgagttgtttgacgcgaggcaacattacgtgcatggacgatatacacgttactagttgtatggtatttcggctaagtttcctagctaatttcattaaaagctgcgcattggtgtttttcagcctatatactgacgtagtaaacttacatacttaattatactaggtaatgatatagaaaatggctgtacatcctttctgaaatgcttccatgcaatggtgctacaagtcttagatttacattataatcggaaaaacatcaacagtatgattacctaggaggagctagcatatccagaaagtagaatagcagaagccaccaacagactgggtgagagtgacgttatgacggatggatcataccccatcttaggagggtcaggtcatttctcaatcatatgtttccagatgcgatgcaaagacaaggcccagaaatttcaattgtaggccaatcgtccggtcgtattaatctcaaccaagtaaataaaaagcatgtgggctgggcgcagtcaaagtcgcttttcttggtccttactaatctgaagaatatacagtaaacagaggatagtggggctagttcagagtaataggcaacaaaccctttcatgcattactgtagaatttgatactattgcgtgtatcgcttttaactttataaagagtcgatacagcgcaggctcataatgtttggagtctgtctaataaacatctaa

SEQ ID NO: 43 - DNA, 2000 bp, Artificial Sequence
  Synthetic
tttatctatttcatatattgctagataaagttgactgacttattacgattattgtcccagacagccgagctgggccgtgcgtcaatgcacgggctcagcctcctaatgttagcatttgttactcttggaacatttggatatagttgattttttgatagtgcaaaggttctcggtcatccggtataacgatactccctaccctagacattcaatcggtgcgatggtaagtccgtttcgcactgaaagcctgtagagtctatttgatgtttacttaatgcgatttgactcaaatgtaggttaggagtccgttcgcatcctatgcagtgataaactatctagtgtgtttaaaaagacgcaaccactaccaatcagaccagcaaatttacatcaatttatgtcaaaacgcccttacttcgtctaaatatagatatatcaccacatcaagcctgcacttctcacactatgttctatgtcatgtcgttgtaccgaacaattgatatttaaccggagttgaagatcagctaaagagagaagttatataaccaacaaatacagcccacccatcaatgatcgtgaaaacaaactgtacttaacagttcaagaacagtcaccatttctcgacgtacaaaagattcttccattatggttcgatacaaattgttcaaacgcctgtctatagcagggctccgccatatttcgagcatactaaatcattgggtggtcaaacagtctcacaaacaggtctgttgcgattcatacgagacgaccatacttaggcgttgaaatgtcgttgcatttaagtaacaaatactatagaccgctggtagtcgccatataactctggctccagattatacatgacctgtttagaaaggcaatgggaagagggcaaaaccccaagattgttcctaatagttgtagataaatggatgatatctgcatcatcactgtttagagaatcccgctttcctttattcggttatactcaccgtttctcggcgggttgagacatgcataacttctatctatcgttgagaattatcaacttcaattcccgagactgtcattatctatagttgaggaaccttcgtcgctgctattgaatagtaagaacccctctagtccagctgatgcttgtggtaactgcactagtaattcatctgccatccgtgcttaattgggcatgctttgttgcatcccactcccgaacttgaaggttggaactctcgttttgccagcacagttaacagggagtaagacctattggtgtgacataacagttaggtaaatccatctaaacacgtgtgtttactaatattcagtcggtggactaacagacaggagcttacccatccgtggatgttttcttaagggtgtcgttagaatgaatagtacatgtatagtactgtccgaggtgtagatagaataaatgtgaccgtgatctcagatttatggttcaaacgttctaattttccgaggagtagtacatgttggtaccttttcacattatggtgctaattaggcatgtataatatcatatcatagctttgcccatactgactatactaaaattgctattttggaaagtttataaggccgtttctcattgtatctaagacctaagcttcgcgtcaagaaatacccttacaatcggcctatttaaaattattcatttgtctagggcgcgatgatcctttccgaatattttatcgattactacttatggatacccgttagacgcttatcctcctactacaccgtactaattacgtacttttttcgaagtacgatctgattagtgtcgaccaccttgcccttaaatctgatcgctcccaccagtacgcaggacacacgtaacggtttcgatacccagcgagatcagccttaccagtgcttgtgtggtataaccacactatttcaatgcacaatgacaagagtactatgttaattcacatgcctatctagttcaattacgttcagactcataaaatgccattgct

SEQ ID NO: 44 - DNA, 2000 bp, Artificial Sequence
  Synthetic
tcgaaattggatcgacggagctaatacgcaaattatttgtttgtgatttctatcgcgcttcaaaacctacaaaaaataacagcctttggtgtaattcgtcgtggccataaatatggcttattctatatatccgaggcccaggccataacaaattctccaagatttactaaattagtacggccttcattccgacgggaagtttaaactcaagccatggagtccggtagtctttcaactttgtcgtatgacggtatgctacatgcccccaatccgctattgaacaatggcaaacactacagcagttagccagagaattacgctctttcactttcctagaagtacacaagtcctgaacctaccaactgactgtacacaccctctatggtacttttgctgtttagttgccgaatgatgcatcatgtctgatttttcgggctagccttagctgagtgtcagcttcaccctgataagacaggagtcagaaacggaatttcattaataccgcctaaggcgaaagagaggctgtcatgtaagccggcaggtttcccccttacggggcccacactctcccctcgctatgaaatgacacttcacaaacagtcgctactcaggatttattccaagttccaacgatgttgagtacattgagaatgtattatattaagctaataggcagttttctccaactatcgattattcggctgatatagcccccatcctgagacgttattacgtcactgaggatgatctattcacacaacacttgggttaccatagttcggaatgcgatctaacgtctcacaatggtttttggtggaagtatagtcttattccccgggctatcgcaagcacccaggagtagtttcgttggtgtcatgcttatccctacgacccaccagagtgtccaatcaatttacacctaaactggaacctaatatattaatcaaactttaaatctctatatattcagactactttactcactttgatgttagatgcgtaacaagcatataaacccgtttgtgatcgtactcaatcgcacccttctcgttattgattgatccttgcgcgaggtaacctgggtaatctctaagttatcgatgcaccgtatcaacattcatgatcgaaaaaagtttagtgagaaggagttaatggatcgttccgactaaactaatggaattatgtatgggatgtatttcgtttgagccaattaactaggaactaactcatacatcttgcaatagtggtagcgtaaaatggttgaacgtagttgaaatagtagggatacgacatgtcccctaagcctcacccttggtagttctcgtaagcggacaacgcgttatcatcacgctttggagtgtactagtttatgtctactgcgttcgctgacaataagaacagcaatatcccaattctcagtactgacgtaggaccattagcgctataaaaaaagtagcgtgaactgtcatttattaagcattccattttatccagtgtccgctaggcggctaaattatacaaacagaacggtgttcttatactgttactacctccacaagtgggatttacgaacgcagaaagagataagctcactctcgctatgtgcaccgatgagtcatacagaggtcatcagtaaaggaactcaatctagagttacagtccagcaatccaatccggatgccaacaggcgtaacgattatattcaaccactaagccgcataaagtatcgatgattagcgggggaatacctcctaaacagtttgaccggaacgtctacaatactttgccggttatcaatgaaatatgcggggacgaaccatgcatcgttactcagcctttggtgtacgccagtaggagtactacttgttcttcttacacgacacgtagctacttctatgtatagtaatgtagttgactatagaatgacgaatagagaagggaaccagagctcacttattccgtcaactcgatttatcatgttgttaaaaaagataaaatgt

SEQ ID NO: 45 - DNA, 2000 bp, Artificial Sequence
  Synthetic
gctactattttagatatgcatcaaagaaaaacaaggacatctcctgtatacgtataggtaataagaagaggatccaacggaaaaagccaccggtggagataataactattgttagcaagtccagttttctgtcaggggcaacgttaagatagaggccagggtaattatttaactactagctgcacttcgacttcattttctgagctctgtaaataccaatggagcgagtagctacggttaaacagatatcggctggatgtcggtggtaggaaaatgtgcctgttgcggctgataagcattaacttacctaaacatagattgttggttttcctaaggttttataagaacgtatataaagatttcttaaatgacaagcttagcctgcataggctacatgtgagtgtggatggcttcgacagtgatcccgcagtggaccagattccattacctgaatgaaaacgttcaattaaaccacttaccgtatcactctgtccttgtagccctgtaaaatgagacttgcggataccaaattagccaaattattcatctaactataatacttcttccatgaaacattaatacggccaccgggaagccaccgattctgtcgccttatattttttgctctatgtctttcttttagtccgacaactaatgtgaacaaatttcgacctaacaaaatagagacaaataaccctatattaatacaacgctacgaagatcttcaataggattggtccgattatagaccaattatacttttacataatatgtacaaaacatctcggcattcgatggcattggcgtggatattcgattgtaaaagcaatggatttttcttgcgctgaaaatgatgatcgccctcgatcatctgtatagcacgggtcgaagtttcagaaatgatagttgctcaatttggttcacttcgaatttacgctgatgtcccaagcgacatgtccccgatcaacatggttgttggatatcaaaaagctgataaaaaatgtgaaaggacacgcctccaacgcgtaactgtttcacctacttccatttcgaggaactgggtcgatttaacgacatcaaagttgtttgctcagacagtcttcctatgaaaatgaaaagtgatctaggagtagaacccgatggctattaataaacacactcttactaaataatttggcgagcatcagagcgtaggtactcggaacctgattgccgttccgctttctatacactgtgaataacaaagtcattgaggtgacaaccttgccgcgtgcacggtctaaagcatgaaattttaaagcaacaatcaaatctctaacggcctatctcaagttacgcagctggcggtaggtgggttttcgcactgactctttaaccaagctgctgctaaaatactcttacctcactgttgatataatggtcgcgattacagataatcccgcacatctgtcaaatagaagatccagtaaagagtccaaatcagagagacccaataaagtaaccaaggcattaccgtttcacgaggtggactttcatgaaagcataagtatggcgtataatataatgttatttggaaaaaagatctccacaacctgttttaccgctgaaaaacctaaataccgtaccagacgaaccacttgatagtcgaatgcgccattgaaggaaacattctccgttaatctgattttaagctcatcaggcttttatctttgcgttatctacatttgacgattaccaaggatcaattacgtgattggactatacttaatatcaatgtacgaaatcgtctacgatactacaaggtaaccactgataattcctcattgctctatgttcacactgaccttgctaatcgacgtggacttgcgtccttgtctagcttataatagtgagatttaatgacaatgctggtataataccgtgcaactacacgcatagaaattactcagcgctcgagaaaagtagattacttcgctccttcggagttttgcgtattttca

SEQ ID NO: 46 - DNA, 2000 bp, Artificial Sequence
  Synthetic
cctcatttgcccttttatatttacccgagttagttcacgaatgtgccataattctggtcgcagcaaactgcggtgtttagaaataatcttccgttattcgtttatcaagacctcgttgtttagtagttctagctgaatgcggtctattaagttggagaagatctgggttcattacattagaacccaaactaattattaagttctgctcattagcattaggtagaatctattcttgtccggcgctgttgctactgggtttagtctaagtagtactttaactgttcctaagggatgctgcaaaatgagatatactcctccgataatgatcaatttggattttgggcagcggtaaatgttttatagtgtgaattgtgttactaaatttcatgacgtaagctgaccttctaaccgtcgtgcttggaggatttacgcggcgccaaaaagaaatatactagtcccaatcgcactaggatttgtttaaaaaaagacggaaaacctgcaaccaaaggtgtcttgtactgactctatctgcaaaatttggatgttctagctccgtttatggtcgctacatggaaacgctattggttaaagattcactataggccagttcaagtttcccgaaaaatcgtgacggacgttatactctaacattgataagaaccatgtatcaagcgatccgcaatatagggaaacacggcgaagatcaaatttatagatgggaggaagcacacacaatatgagtattagtgtgctgaaatcagcagcgtaaagtgcttctgttccacctatacttttacgagtctcgtaatagcgtattaccatgtaagatgcattaaagctataactttatggcaaaaaaggtaatttattcgctcattactattatttgtcgttttgcataaataaagtgttgttacttcaggaagctttaattctctgtctgccttaacccgaattctacgcgatctccgtatagcagatgagaaccggtgacacgagacccgcactcgcaagtcgtttcttgaggctaacgacaaaatgaagccatcagcgaaatctcatccgttaggctacccaaagttaagactttccctgtatcccgctaatgcgtcaattggtagacgtatcgggattagatattcaagaccaagtcaggtagagttggcgctagttgaacatggacctggccttacaaacaagaagaccacgagagccctagtacaggaatttatcggaaaaaataagaaaattaaaatccccgatctgtgtggtgctcaaataaggcaagggcgcttagcctcacagtcgttactaagtcaaggttctaaaagcacgtgttttagcttgatggatcatgacttcgctacggtcactactccaccgtgtttctggaggtatgcaagggaaaatcgagggatgtgctcaaatctgtggcaaccggagcaccattctaggtaacttccattaacttttgatttagagtatatggttaagctattaaacgtttcctaaggacaagtgggatagtgatatacttttttcggcgacatcaatccaggattatccgctaacagatcgcctagcgctaccgcatatgatgatatccttaggaagagatccaccccggccaagaaactccacactcaataggcggtgacctatttgtgagttatgcagatgtgtttcaagactcaacgccgacaaagttcaccaccagagagtgtaaggcttatcaaatttctgattttatcgacttataaatttgacacgtctaacagattcggcctttgattgtaaacatcgccgctatgatattttcgtgatcctttgggatacgagatgcatcagtactggccccgaatatttccattttaattactgtgtaatgcttaggttcacaatcaacaagtagttcgtgaaaatgttactataatatccacacaaagatttacgcactctaatggtggacgttggacctctgttaacccgctttcgttatt

SEQ ID NO: 47 - DNA, 2000 bp, Artificial Sequence
  Synthetic
actaaagtcctggagagtatgcttggcctcgtgcggtaacatttgaacagcatgctaggtgctagtagaccctttcttgacagcggaatttgctgttattcaaaccacctgtcaggccaattctggagcgcaacccacagtgatagaagatagtcgttacaatcaaatcccacaacttgagactagccctcagactgcaacagtacacagttatgctgtggagacaaataaaatacgttatgtattggtcattagatttggctttcttatacgtcgtgtagtaatgcttgtgatcggttgccgacatggttacgaatagctgtttattaatttaaaattcaattctgtcgatttagaggatggataatatccgctatgtagacatgagtgagttccttatccttcaattcccttttttctgttattttggatctacgaatgaggtattaagttcgtagcactcgtccgtttcgtggaatgacttattcgagatggcttgataaggaattgtacctcaaaggtttcattgttaagaagatgaattttcacgcccatggcataagcatatgattacgtccactaggtcatagacacatgataactcgtcgctcaaaataatcgaaagaacgtctatcggccaaattattactttgatcccaaaggagaaatcatattggggcgcgggacttcatgtgtattaccatccagcaagcatttgataaaagtaactcctatattattatgaatagcggtaagtttctttgaccaacctgacaataacaccaagtgactcactgagcccgttatctactaggtattcgcgaataccgtaaaagcttgatgcaggtgacaatgagaattatcattagcgtactgtatgctcaacctagcctccttgcaagatttcgttctatctattttgtattcatttctttccgcgacatgcattcttttgctagatcctgggtcctgcaatcatttataagcacgcaacttagcttaaaagtgtggagacgagacgtacaatcactacttcccatcacttcttctcttataagcgtaccgaaagacctcgtattttattaaacaataacgtgcagttggcctaacataattcgatgtctttcagtgttctaggaaaggtgcggtgtgtctagcaagcatgtcagccctacagattcttaacatacctatgtgtctaaatcgagtatactataatgatgtaccataagcccttgccaaaggatcatattcggactagttattgccttctggatggggtacttagactaacattttaaacctcttgcgatacgacctggtgctaatacactattccttcttttctcacgcgaactttcagtatcgtacaaaagtatgggatttaaaccttttgaagtttggtcgtgattatttgtttttagggcctcctcgacgcctcaaatagggatttcttcagcactacatattttgagccgtatgcgaacccttcttaggaccgcggtagtttgttcacgagcacgttggccacaccccaattatccagaaagccggacttaagacatattgagtttgttagtgcataaatagggtcgcatattgatctgcgactcgagtaaatgtcgtactggtgatatattctcccgttttcgaaggccccaatcaattactaattaccctatttacgaatgtcgagagatgttcaaacgaaacatgagggcgcatcccaacgcccattttgaaacttgattgttgtataattcttaatttttgtagattcagcgttcttgacacattttaaagacgtcagttcaccgtacctaccccttcggttacgcgaaaaagattaggttaacgatttctatcgttcgttggttgttatttctgcagtacattaattttataacttgatatatcaaatctgtttttgattaatgtttgaaagcaaatcgtaacaccaaggaatgcaaataatcatacgtggcggaccagctactata

SEQ ID NO: 48 - DNA, 2000 bp, Artificial Sequence
  Synthetic
tacatccccatcagtcaagacgattcgttaacaaatatcgctgactgggagaatcccagcatgtcttggctggctaaatagaagctactatgttacgcacttccattttgaattacaggcgacaacattaccagacttagttaattattaaacaagatcactttgcgacagtcctctgaggatcagttagagtgcaatcacttaagtaatacaaaaatacagaaggattctctggcgaacaggtttattagcgcatggccaaatttctaatcaacccctttagttagtacccatttctagccaatatcaaatgtactccaagccggcgtatagttgtcagtgtgtgatttaacgaataggatccccccccataacaaatactaataagagtggagcaattatagtttagatcgtaaaggtttaaataaataaacgtcaagcacaattatggactcgtatggggacaaattgagcctactagcagttctagcgaaataagttgacctaaccagtccatggactgccggttcgttgaagtcggtccaacggattgcagatcattgctaggcagttggtagataaatttctagtacttatagtcacgtaattgtcaaaagtcctacgagcgtggtcaccgtattactacgacctccatagttttctaccgtgcattctgaaagaaatatggctggagtgtcctagctcatgatagaaaacgcctacacttagccaatcagacattaatgcggtaacggatcaagcattacagggcggattggtcgcatatcattgcacggaaagcgttgccttaagttcggtacattccactttcaacttcatattgactcaaatagtgggacagtgatttacgcggagttttaatctaaaaattcttgagtttatgatagaacagatctaaattacggtttttatatgtagtggtattaataatgttcataaccctagatatttccgagattagcactcgttcggcgcattgccggtatagaacaatatgtgaagaaatttgcacctaagaagttgatattctcctctacatgcgtataatatatagtaccataagtggatcattattaaaataaatctgagtgggtggacttatcttctgtcaccctaactggatcagcagtgggctagtagccattaaggaacaaccacttggcccgaaactatttgaaaagtgataaatacatacacgatttactacataaccactcctcttgttgataggcatgcccaaggattcgtatgggcgattttccataaacctacagggtgattcgcgcatataaataacaccaaagcagtcaggctttttgtatgaagtgtagcttccctaacagtatgatagttgtgtagagtcgcttctgaactggctgaccctagttataattagttcggcggaggatgggccgcgagacaaagtatactcgaaccttagggccgcattccaaaggttatttagataaaagtacgcaaacccgcacatgagttgaaataatgaagtacaatgttatttattgtgcgtggtaatagtctcgtgactgaaaatttttacctttagggttctctatccggaggagcgtcatgagctcaaatacaaaatcggagcattgactcaattactactttatgacaaattctacgtctaagcgatttttctaaatcgccgtgatcaacaaactagatctacaccagtgatgcatgctcacggcgaatgtcctgaagtcagatctaattcttaagggttggattagctggctatagcaagccatattaatatgattagtcgtgtatggtttacgctacctctccatagatatttctaacttacatttgtaaatgtttccaagcataccgtcagtataaatacccaatgatgtggctctccttcaagtgtttagataatagctatttccataaggtgcctcccctatccgctcatcctcgggtttcatatgttgtaagtggcacttaga

SEQ ID NO: 49 - DNA, 2000 bp, Artificial Sequence
  Synthetic
ttgtttcttggagggttacttacgattattcaatgtcaagctggtaccaaataatatgttaacatcgacaaccttgctgattctttaactgtacgatttactcaatccttacaacagtctttccccccgatgcttccgataatccggatggaatgtaaaagctttaatttagccataatggagctactctgcaacagtaaggcaaaattttcttaaatggaggccaggcaagatttgtccccgccagaatagcctactccacaatattctctttaaatattcgccatgctatctcacgcatccatgaacaggttatgaaagcgtagagtcaaacgtacactttaggttaggtgccttgtggggatttcacgccacaaagtagagtagaagcagtgtatcaaactatgtgtaaaagtaatttcatatagtaatagccaccaagaatgcgaacataggtgtcggcctgaagatctaaaattatacttattaacaatcatgtgagtaggttggattttaacacgttcataagtatcgatcgcttcgcttaaatagaataaagtacacatcatgtgacgacgcgcttcgattattgtgctgcgttaagagtagtaggataatttttgatagacctgtctataacacggtatttaatccgaagttcactatacaatcataataggatatcgtgttctgtctcgatgatctattcgtcgcttcgggtgcaatataggattcctatatgaaactcacttccctgagcattgggatttcttgatagctagatcgcgttagagtcgggcggtgtatagtctcggatacaagaacataagagtaattatgtggaaccttttcatgtgattgtgctaactgtgtgatattcgcaataattcctacatcttagtttttagactggacttttttttcccaagctctaagcatacattattcgctgcgtatgtcactgacctagaggaataagtgttctgctgtcaaaactaactctctctagcagcctttttgaccatattatcaattacgcgccatcccataataacttcaaaatttgcaaccatcggaattagaaatcccgacgtaatcaagacgaatcttcgccgattatcgagcttacataatcgaaggtgcatttctgaaccttggctacgctaaccctctagtcggggcaagatgacttggttatctggttaactaggaactcctagcctcatattgtatcaatctgatctaatacagcgtctaccaattatttgattaggtttgcttgccctcatagcatcgcagcgagtatctcacaatgtgtatgggtattcttctagttacgagtttagacggagaataagccgcttgtggttaacctctgtaaatacctctagttgaataagtgtgcaacccaattcacattcgtcatgttaacaaatcggcaatctttccactaatgagaaaaaacaaatcattaatatatgtgaaagtaattattgtgtcctcataacggtaaagacttacgagtaggtaacaatctcaacttcaccaattaccacctagattccagcaccgccaacgtaatcagtgttccgtgcgtcttacacaagagaactccttaagcggctagcgtatacttttaagagcagtgggtatgtggcccggggcatctattgtttaccgtaatataagcgcactagtctatttttacactaaatatcattccatatccggttctttcagtaacaaaagtaaacacagtgttttggaagcagtgtatcaagaattgtgaacttctttcaccggcgcagggatccactgtctagagagaatcttaattctatcaaccgaccctccatgtcttatagattgtgtcaacggagcacctaaccgtatccttaaaaatttagaggaaatagaactctcattcttcagcctgttaagccaattaaatcgaaaccgttgctattaggtgtaacggtagatgtgataaaagggtcaca

SEQ ID NO: 50 - DNA, 2000 bp, Artificial Sequence
  Synthetic
aggacgagctctaggggtgcccctgctgttgttggttatttaaaagccgcgatgaagagaacgctagggggaaaaaacgatttgcctagaatagtggatcggcgttttgatgtaagtgtaattgggtagaaggacttgttttacatttgcgaaatcttgctcggggacgttataatatggccttgaaatggatgatgacaatatagttttaatgttattataattagagtatcgtattattaaaaaggatgtccactgtggatccaagttaagcattaggcgcgttgaagagattgtaccgcccgaaccaatgcaattgacatgcctaactagcaagacaaacgtgttaagactaaagtccctcctatcaacgtacacctcatacgcttgactaggtagaatactaaaatactctcgtaatgaatacctattatctaagtgactgctgcgttcttttaggtggtgaactggctccggaaagtgtgctaatagtctatatgtccgcgcctgccacgtaaccacgaggcggatcagctagaaacataaagccgtttgagcaataagtgactatacttaacggtctgtaaattcgcgcttcaatacctcttactctctgcgttctatcccgtctttttataaattcaactatacgctccattggttatcgccatatgagtccttatctacttaaactggctaccaattccttgctctaagctaatgaaagtccattcgcaggattacaacatcaatgctaactttctcttgcatacagtatatcgtctaataaatgtataggctcccggaggtcggaacagcagtactcccggccacgtatcccgaatacaaaccttattagtaaaggaaacactagtgagagcgtacggggattactcgaaatatcgcaggaaggtggttaatatgccaaggaaatacgaataattctctccgcattccgaaactgttagcacatagacaagacaaagagtttactgacacatcttttgacaacccgcactctacaacgacctactctttatacaagtacggattattgtaacgctccagcctagagagagtaacccggagttatatggagtcgcttgaggagaaatattaaagctgaattctgttacgactagtaacattaccagccgaggtctgaataacgtgcctatggcgatcaggacaatacgagagaatttcttctaccacactatgtgcagcagctcactcaagagtcctatgtagactgtttaaccagtaaggattgttgtgcggaagtgtaatatggtcgagaaataccgctaatatggataagttaattgaacttcggacgtcacattctcctataatgaggatctattcaaatcgttttgaagtaacctcctcatttgagtaaactaggcttgcctggagatggggcccccaactgtaatgtgttatgtttagtttgaactcagttggctcaaagtatcccgcagtactaatattaaatcttgttattgtacagctggcgaagaaagttaagaaatgtgactcctatactattactggatttacaaagtaagcgtctttgacattaattatggtattgacaaatcaaatgagagacagtaagatgatgacattcgctcatattgtatggctcgttgactgatgcaaatagtaccaaaccctttttttagaattccagatgaggaattagatttttcagtcaatagttacttgttatgccacgtaggcttatgtcccctaaatcgcatataataagatagagtgcgaatgcgtgcacgtgtacactaatcagggcaaactaaacatttaacctttggagaaattccgtggcgctgaacttagtgatgatatatgattaagggatccgttttgttttcgataatctaagaactgacgaaggcactaatatcggagttacacaggaaatagaatgtcgcaagatgtgccttaggagtcagaaatcaacgagtgttgatcccaca

SEQ ID NO: 51 - DNA, 2000 bp, Artificial Sequence
  Synthetic
acaacgactttcgaaggtggctgaagaaaaccacatgataaaatcgcgagtatggtaaaattagctacctgagtatatttaatcgaggttatatcttttgtgagtcggacacaaattctatatttgacggagcatagggcagacggacatataaaattataaacagtctgtacggcggggcctccaattggattcccgcgatcatatcagtcagttgggaaccataaattgcgaaactcagtactatgcttcaatgcccctttctaacacgtttatcgcttcaacctaacggtatttgcactccgactatcgtcttatgcctcacaatcagatgtaataatgcgggatttataaagattttgaaccattggacaactgacggcttctcatctcaccttgacgagagtatttcctattaacctgaatttcgctaaatacttatctttatcgccaataattcctttatgatacacagggcttctccaattcatccacgcagaaactgcccaaatgaggagaataaaaaactttataattaaatgaattttatagcctatgcgtatccccctacttcaaatctgtgcagtgatgataaactattgtaatgaagatcatttaattcgcgagattaaacagattcatgttctaatgcgattattctggtgtgatatcgtgcatggataatagaaagctgatccatttagaaaccaagcttatgcctatccgcacctttaacacacgcatagattagcgctctgcgcgaatcctgcgcgttgcaactgtactgatacaatgcgcaccaaaacaacttatactctagcaatgtacacacatattgcgagccaatctgttcagtttccctttgatatttcaggataatcagatggacgccaaatagattactcttatactgaggaaaatatgaagttcaggttcagcgttacacgcaaatcagcgattaggtctgcctaatatgatttacgtaaataaatctaccaactagaaatccggatattttacaataatcatggcaacgggtatgaccactgggttcgatccatatacctgatgggctcggcaaaagtctgtaagaattctctacatcccgatcgatgcttctttatttattttacttcataaactcgtatttaagctatgcattgccaacagggcttaaataagaaaaagtgttgcacacagaagttgctatgccgcaatggaaagagtactttcatgaaaatacgtagatatttaggagctttcatttagtaggtcatctggttgaccatatactaatcggatacttgcgaattattgtcctttcagcagtgaatcctgagactgataagccagcaggcgggaatcgtattagtaaaatttaaggacatctgagtacgggcgaaatctacaacacgacgaaatcatcaatctattatgacataagtattggacagtacgtctgactgggaaacatagctttatgttggatatgtacattagtgcaaatctgtgttacgtgttaaatcatcgcgttctagaactcttaatcacatagcgagctaccttggcgaacactcgttactgttctcgttttgctatcatgtcctaaaagcggcaaaagttattactgcaggaccgaaaaatatgaaaaacttattttttcatgggactacacaaatcgagttgagcctttaagcggttctatgttacttgagtatcttgaacttggaggggggttataatgataatagcaatacataggttatgataaactgtcctgttttagatacacgggagccttagtaggcttattttaatagtgtagttgttgatatgaataatatagaaaggccatggaggagaagtgctatgttaagagggcagtcgcggtcacgtgtgccattgacgctcacttatatgctgcgttttcgcagtgtctcaaagattaaattagccatatggtgtctattgttttcgtaaacgcctagcatgcgttcgtc

SEQ ID NO: 52 - DNA, 2000 bp, Artificial Sequence
  Synthetic
cttgtgcgtcgaaatcgaaactcaaatagtatgtacgctgaaaataataaagcctagctaacaatccatccgcgtttagatcgtaattcacattttaccgataaaaagttaagtacaacattggaattgttattacttagccagccaataacgcgtcctaattaccaaaaaaaacagactctgaatcatggtagattaattgggtatcgataacattatccaaattcagggggccattcgcttaagaaaagagatgttaacgtactccagcgatctgcggtgttctgactgtaaaaatacgcatacatttcacccatagcagaagacgtaggacgtcttttctaccaggtgtctgtattacataccccatgcatatctaaaaggattctggacgtattttgatttttaccagttgagatagtgtcaaattctgactttcaaatgacaatcgcaaaaatgtatgcgaaggctgatgatcttgtaatcaatactggtgctagtcacatactgttgtagatacgccagatttacactatacacagtgaacaaggtcatgtcaataacaactatttttgtttataatcactaaccctgcatatgagggtcttgatccaagttcgaatggttgagaattccgagtttattggtaagggaagatgtatcaaatataatccttgcttacttcccaacagtcacaagaagcagagttaacgactgattacggctggaccaataaatattgaaacatcgcaataaaacttgaagaaatttgactacaaagtttaagtgtatacagtagatcggttagggtatactcaattagggcggaacccgcattcctgtcgataagctagtagtaggtggttttcaggttggtatcaaccatcaatattcgacatacattaatccagtgaataggggcgtccggattttgtaaagcattaaccttctgtataaatactgccaatcatatggcttgagtaaccgtttttgtcagtggaatcgtcccctcgctagaagcatctgtacgatatctaatggctgtagttgccttaaatcggaaaggtaagtcggaacctgggctctcattcgaataagaccaatcctaaacggcgaattcctttatcttgttaactgctgtgtcaagtcctcttatcgaaaattcttacatgtttactcttgcgattaactatggtgaactaatcccaacaatgactgttcgtaatagatgtgtttgtaaaattagtattttggtgacatctctagtcatttcatgccttcatagatcatcggtatttcgcaataatctgctcatactatgtacagaaataccactaccttctgacacccttgctagcactctggaactaaataactcatagacgaaaatacaatgcaaagctcatcttcttttgaatattgagcgaagtagattgttgacgttaagaaatgagtagtttcattcgagaacatccgtaatcaactacaattataatctcacaagatcggtctattaaatcgctcatactcctaggactagaaccaacgatcgaatttgtgctttgggcttaggtaaagacgtataatcctacctagaagttatccatttatccacttgataacatatgtctattccccaatcataataagacgtagaagaaaacgactctcacaacgacagtatgccctaatatgcgatggcgactgaaaatcttacggcgcccgcctcaatcacgttcacgtgacccagcacattagatccaggactgactcaagatcattactcggcgatcaacgcactatcctcaattggctatgtgcgaactcctcgtataggataaggatattccggtctccgtatacgctaggctcagtaacgcgtcttactctgggtcaagggtttaaagatcatagcggtatcatacaaaaaatcatatggcctactttgtcgttttaagcgaagatcaacgacgtaatagctaacttaatgagcaagattt

SEQ ID NO: 53 - DNA, 2000 bp, Artificial Sequence
  Synthetic
tcgataggacagataagtgaccgcttgttgagtcttatatgtattggacttaacatcgagcaacagtctgtaacatatgtcactacgtgattgaaggccgtcgtcagtaattaaggataaggcggtaagacataagataccgtacaaggatatttatcgttatctcaaggtcaaatctaactataggtaacaattaccttctactagtaggggaattccgttggatagctagtaaaagattgcttcaactaatccaacaaagtattacatcaaaacagattggttatcaagattggagcttcagaactagagtggtgagcaaagcactctcatgccttttgtaagaaccgggaatgaaccgcaagaatcacttgacaaaggtattgggtggttatgttgccgggaagctacgattatatccaataggctacggtcgttgtacaaccggttgtctatctggtacttggttgatgacctaggtgcgagccattctgccaaatttatatggagattaagagtggtctttgcctgatgaaagggccaactgccgaagtactttggagcagtgttgactgcagctccaaacatcttgtattttaatatttcggaatagacatctatcgttagtgaggaaagaatttgatcccgcgctattttcccgacattctcaacacttggattacttaactcatagaattttctacctattatattataacaaaaaggtcagtattggtcctgacgtatctgattcacgtattacggggcggggtggaaaaacttggtttcctagagccttagacgagcgttaatatacaacaaactagtttcacataatattacgtatggagtagactcaaacaatggatcgcggcgacgtggatggtattatcgcatgatgcaattctaacgatgaatttgtgtccgcgctgttgtcgttttaacaacgattttgaggttatgatagttataatcattagaacatgtccgaaattcaagtggttcaccttagctttgtcaattttgtcacacttcagggagggtccaggaggaactgcaatcgtcagtctgaatcgttcgagcagtagaaatgacctaatttgctcgtgacgtactgacgataccaaatcaatgattgagttcgaggatctgatgtttggagcttgcgttggacgatctgatactcaaaagtcgacactcaacattttttgccacgacagatattctccagacttaagaaatccttgctgaatatcaaacatgcagcttagattagttattatgtaaattgtgagatactatgctaactcgatagtgaggtgttggtctgacaccgtgaattaataggtcgtccttaacaagtaccacttagattcctcgcttttgagtctttgacgcctttggccggatgcatgtataaatccttttcaaaaggctgttcattcccatccaagttctgtaataggtctatctttacttctggtaacaagagggagttgggttacgacgagtaattgttgtagcaaggataaactgctatttttgattaacagcctcacatataatacgggcagccaagtcagcctgccggcaaatttagcagtgtttctgctcgccaatgtctcgagactcctagctctctcgtccattgctgactagaactagccaattcggcgagcattagagtgctaaaaaaatcggtacaggagcctaagggtatccgggcagaagcaagtggtgccaaagacagttagtttatgagcttacgtccaatgatagaatttgcaaacggtatggttaccttcttttctgtatcttctcaatgtaatatgttaatgaacacattgttaatgtggtttcatatagtaaagtagaaaactagccgacaaccaaagtaagaggagcagttttagaatcaaatacaccaacttaaaaatttgcatctatgtttttgacaattgacatacgacataataaaagtaggatagttgtagatcgtc

SEQ ID NO: 54 - DNA, 2000 bp, Artificial Sequence
  Synthetic
acaacaatccagaattaaagagtcaatgattaaagtctctataattcttggtggttaaggtgcaacttttgtcaagccaatgcttctctagcttacgaaaggaactagtattacaatttgttaccgcatatactaatgatcaaacattgtacaggtacggttaataggcgcactagtaacaccgtcaattattatcctcgtccgacctgagaaaggatgatagatcgtgcatagagggacttgtggaacgaagaacatttcctacgcagctacaaaagatatattgcaccagggacgtcacactaaagatgtatactacagcattgtttctcataacctctaggtaggtctgtagattcagcgtatatcgactacctacatctcgtctgatattcatctatcgccttaaaattgtgtaaaataatctgaggtcatcaatggttttgtttttacattatgtaaggtccgtaatggtaacttgtgaaccgacatagttccccgtcgcttaggtgtgcagataattagatccaatggatcaattctcggagatagtcttctacggcattctatctgtacacgtattggtacgggggtcgtaggcagggagacatctacaaaagttagcggttgctgaattattaatatacagctttacgcttatacggttgactacaaaaaaattacaagattcttcatgagattgtacctgtcaacttaattcgtatcaaaaattctaaagtgcgcatctaacttcatacaacggagaaaagtacatataagtagggtgtgaacgcagataacgttcaaaatgatttaaactatgattgagatgtccaagttaaggacggtagggttgctaccgtggactataaaccctaatgcctaaatctttatattcgggaattgtttcgggttagggggaatacgcacgaggctaacacaatatgcatagtgcgtatcattagcgtatggaggacgaaaagagatatacccaattatagcctgaatgtcttaatcagacccttatcgtcatctcatttttgactacaatcggtaataactactcgggtttactagatcctaacgggatgactcataatagaacgaatagtgtaaaagcaacctacgcgtaagaccttcccggtcatgaggatgtcatcctatgcaagcgttcctcccgcgaacgccacgtgatctctcgattccattctataggattcattaaagctctactattaccccaattgctgggtgttctaagatctataatgttattgtccagattaagttctcctgcactactcgcgattgtgtctttcgcccgcttgtccccccgtaattggatcgggccttcgcgttctgctaatatttgttacgtcacgtcggataacccctacttgtgcaacatcctgacgaatgttgtaaaaagtttttctttggaaatttgtacagttaaaagacaagataatatgattggatggcaagtgactgtaaagttctatccagtgtttcgtatacgattaatgaaactaaacgagaaactttgctgacctccacccaagatagccttcactctttcactaactccacggtgaattttttttagtaattttcataaaggcaaagactaagtttacctagtaacgccaatccccccaccatagtacactgtgattcgaaaaaaggatatttttgagcttctatgctttagggatatttagtttaacggaaagcaccgtcagcttggaatattaaacacgcacatgatttatggacccatagttgacatcaaggtctttgataccgacggttttcgtattttccagtgaaagccgaagctttacaaaggagagagtaattgagcaaatttctcactgcatgtcacagggactgataaattagtccaaaaactttattacgtttgaccttagaggtaccctaatgcggcttattatttggaggccagactattgcgcgtaacaggctgtttgagcatcggt

SEQ ID NO: 55 - DNA, 2000 bp, Artificial Sequence
  Synthetic
ctcctcgagcttatagaaaagtcaacgaatgtgtagaaccaagaaagtgaccagctatcaaataaataacaagtgagaggtacagcgtatctaataggcgaaagtctagctccaggtatcggtgaagtctaactatgaattaaacgcattgcgtagctacatggttttacacgcaccattaacaggcgcataactactgcctgaatcgctctgatattaaagtcaaaggaagctaaagacttgctatatcgttgcatggtgttaagtaaatacgactcgagtattttaaaaaatcctctgaatcgaccaactatttattcgttcattctctgtcattgagtagcgctaatcaatgtagtatttggatcaataaccctctgggttaggcgactacatgagtacccttggaaaaactctggtcgagcaaaacaagacacatggggttaaataaagtctatacagtttataattatgcaaatttgacgaattttgtacagaattttatctataatcttacgggggtatacatatgacagctttccggtgttacaatactccttgtgctttgtacacttggcggaaaattcaccacaatgtatggggttccgcgcaagctctctttttcggtaatctgggattccttttttgtgcccttttacataacaagacgaattggtctcctttttactcagaaagaattataatacttttcttacttgtccgtttcccctcatctttttttacctccaaatccgattcatcgccttaagtccagtgtcttccaatgtagtggtttaacgcgagctacataaccatcccggatgtatacgattctacagcgtcttgaaaatattatgtttaggtttcgggtgaaacgcacctagaaattatagcaataataatcttaaatctcctcatcataatagataggttattgataggcgacatgaaacccagcggattcacctatcaccaatcaaaccacagttccttttgatgcagtcattcctacaggcatcctattaacaaacaagcgtgtgccgatgaagaattcgtatctgttaagcatccgacggcacatgtgcaagagtcgatctcctgataccaattttagtacttctcctctgattaaaacaacttccaaagttccaacagatggagtatagataatcaagtttccagaattaatcagtaatttgacaagtggaagcgctagaggactattcccggtaatactataacaagtaatagtgaccttgtgtataaatagacgttgatagatatatatacacttcttgatagctgaggtagacgttgatacaacccgcaagtgagtccattaccttaggccctacgaacatgctcaaacccttttatgctttcccagactcaaaatcaatacgtagatatattgtaaccgtatagaaaagagcttctgttggatacagtggtataacagctcatgttcaaggtttatacggtatgacaaatgtgattttcttttatgtgagataaccgaaccaatttcgaaagattactactagttgaaataccaattttaaaggtatcctttccattagaccccttatattattctactgtattagcaaattttagaaagttcgtgtggtactcaaatccgatgaaactattcaccgtgaccattaaataagtttgatgatcaccgagaattcacacctcgtaaataacacctatcttaatagaattcgtgcgcagctctaagagagagcatcttccaaaacgaagagctgtttacaattgctgccacgtctttgatatacactcttttattgtccaatccgatgtttcacaataggatccatggttccggttacttcctagctaaaagggtttgcccacgcggtgagggaagtctgtcggtatattagacgtagtgttcacgaataagtaagatttttaatttggaatggtttgcaacaattacataaggataagtaaacgcgccgtataatgctcta

SEQ ID NO: 56 - DNA, 2000 bp, Artificial Sequence
  Synthetic
attcttaaagtcgattcggtgtcataatagggttatctaacatatgtacaaacgccctataaagttattatcggactggtgcataagtaacagttcgctataaagttaaatgctatcaagagaaataaggcatactgtgatgaaaacgaggtcgtacagaaacacctgcaggaattaatctgccgtatcatacaaggaatatcgttggagtcaagatgactgcccatttgcagttgtcatcttaactgatgatggtttcttgcttgatagcacccgcctcagtaaaaacagatggaacactccaatgctagccaactgaaatttaacgttagtaccaaaggcatccaagcagtcccctggctaagttggagtgtggcatcgatataaaatagttaaaaaaacggtctgatgtttcatgcagtcgcaaccacgcatacggttccggttcgcaacgattgatgtggcggtctcagtattttacaagttttaacatgtcggcagccgctaggtagatacctgcaccctgtggtttcgtatatagggaatttcggtgctttaagataaggattactcataggggatattactcgattgcctcgaaaaatgcgatgagtctctatattcaacggtctattacaggctttctattttctcgggacgcctaggagttgaatgatgcacatcattaagctacttatgcggtcttccataccattccaatgtcgtcgaaagaggatgcagtgacaactcaggatactaataattccttgagaactgtctatttcaagcctattctaacataattagttgctagccatataagaaaatatcatcaaacagatagggttgataacagagggtgctgcccgtatagtgaacatcgtaaccgggtttcacatcctagattggtggcctcctactatgtaagatgtagttatactgaatgtggtgttgtgatcaagacgtaggaaaatttatcagatatgccaactagtatcatcctgagttataaagggggtaatttcggacaaaggtgttgtttcaaaaggttcaagccgacgtacccgcacatcaacttatcttgtaatgattcaaggtttatgtagcttgatcaccaagcaacccaagcgagctgtaccagatacgattatgttaataaaggtttggcgtactagacttaacgctaaggtttcgtaatgtaacgcctgcattcacgtcaataatagctcagtatgtgagaagtccgatgctgttaattctaataacgctcccacttgaaggagaaagcgggagtaggtgcgtttgttcagaaaccacttaagcggtttgtttgtacgtacaaaatttgcttttagatgtatagttgtatacataaccatcgtccgaaagtaaccttcatatgaaactcaaaggcattagttgggaagcagtatgtggcgtttgtgacacatcgggattataaaattccaatatatattctaagtagcagttaaatgaactccactatggttaaatacttgtacctatcgttattcgcaattgtgccacttttacatagattgtgaaccggtatatcgcgtggtcaagaccaggcttcaaagctgtagagaactgtttattctttgagtgacatagtatcgagacttgtataaacatggatggtacacaacgttggaaaagccgaaagccaataagatatttaagcattatgcttttatgtcaacactgactttctaaaccacacaccttaaatcagtagaacagcattttgaaggagtggctaaaccatgttgcgtgcaattctccgggctcgtaaaaacgtgtcgtgctaaaggctctaaatctcgcagtaaaggaggccctccaaactaacttaactcattttgacgaactcaagtagcttctattaaattcgtccgaataccatgaagaacgggattcgcatactgcgttcgccgtagtggagctcgttacaaatcaaatggatcgataaacaaacg

SEQ ID NO: 57 - DNA, 2000 bp, Artificial Sequence
  Synthetic
ttagtatagttaagataatgcgtcgctaaacaacataaagattctttaccgatgagttctcgctggtattcgcttttttagtcttactcgctcaagttatcttgagagatgtggaactgaaccacttgaggtagccccatcaattataaggaaattgaaataggatcgaaatattctgaactatttccatctagtctactgaaattaacattgacacctttcacaaacgaatggcaaaaaaggacggatccatccccacagacaacttcgtttatttcagcacatttgtccctggacaacagccgtatgtggttcgacatactacctgatagtgagcggttatcgaaatgtccttgactagctactaagaggctttatacaatattcctacacacatagacccagtagatatgagttctagttggagatttttcaacacaattacgccacgaggtccgacaacgtatcctccacagttaggaacatttattacaaggaggttagctccgtgctacagcaacacgaattactccaccgtgttgagcaggtaaacgagggcaaaatacaccccaaagcgtaactgcatacgactttccgctcgaagattgttaaaacaagactgcaatttctgtggcaaaagacactaaagatgacagtacagcacccatggagagtttgtacccggttcgacctaagtatctgttgtccagaatcgtgaaatttgaagtggcctaaaagctgagacgagtatagtagggtggaggtttcctatatgttggtcggtcagtaaatatttaaaccacgggagttaaacttatcttaaatgtatctatacattagtatataggctgagattcgatatatatagacgccaccccgagaaatagaaagatagtgattcaaattcctaacagttcggagtggtatacgcatttctgagtaatttggcgtacaaagtttgagtagagcacagagttgataactagagcaatgtctgagagtggattaacttggtgtgctctgctagaaatccccagtgatgatctctcataaaaagtgactgcaagactaggatacaatttattatcgaagtatcaagatcgtgggttccttttttcctggtcaaagatgaatctgtcttacttaacgaaacacaggaacttttcttgcataggcaccgatcttgctatgtattgaagctacttcaaaggacctatcagcgggtgtacacaatgtcggaacatgcataaatggcagaaggcgatgagtcatttcgcacaccaacaggccgacgagcgtaggagcgactcagaacactaccaactatagcataacgataaacggagaacgtccatgccgttatgtgaccattcggttcggagtcgtgggttaccgaccacgatagaacatggcacactgctttctcacttccccaataagaaacaccctggacgtatacctcgattggatctggagacagtactcggatccacacctaagtagtacctcactgtgggcgatggccaagacgcgaggttgactatctgcgtggtggaaaaggccgacagatctttatcaattgtagtgagctgatgagtcctttatccgttataagctacttttattgggtaatagatggtgctcttactccttcgagttaatatatagaaatcaccgcaaagttaaacgcaacatgagtggtttggattaacaacttctggaatcattataaccttaggagcgttctagtgatgctgaaattgagacagtaaaaagtgcccatgatgtaggaaagtcactataaagtgaatctcttgtccttaaacataaagcgcggtaaacactcacgttaagatggttgtggccacaacatgactcttgtggttcttgacgtgttaacgcggtggcactagcagggatgatacaagttgatgcttacccatatgattattgttccccggagccaccactaagccactaaatgaagatttttgcggcga

SEQ ID NO: 58 - DNA, 2000 bp, Artificial Sequence
  Synthetic
gatgttctgaagttccttagcgtacaaacacaaaacgtgcattggaaaatggagagggaaccctctatgtctgatgattttttcggttgagctaattccagtgcaatcgacaataagggcatgtccgaaattcgctttttaatggtagtaggtccggcatcattatgttgtcggcctaaataccataatcattgctcaaccttcaactctttgctggaacaattagtacttttcgtttgcgcttaaccatgcgtataatgtaataaaagcaccagtttatagatatcggaaaatttagagttcatgccatagtttgaaccgacggtaggtacctataacgtcttttgatttccgcaacctatgtattgtaagcagttgtcctaaggagtattttcactgtctaagtggtaaccagcggcgagaacatagtcggcggaacggttctgatttcgactagcatcggcgacattgccttgtcaatctccataatgatataaacatggtcttttaactctcacaacctaaattattaacaggtcgatacttctctggcgaggttgttttaaaacttccactccggataggaatttcattgaaaatataaaaggttgatgtgtcaatcgaagtctaaaaagaatgaagattagtgtcgcctaggacatctatttgttttaaagtgcaaggaacgtgttcacgtagaattgtgaaattggatacatgtttagtgtcatgcattgtttatgggattgactataacttagatagagaactagttacccttattactttgcagtatatgaacgactgattgtcaagactgagcctaaattaaagtaatcagcacattttggatatggataggagctcagtttctggtttcactctcatcgacttctttgtccaaatacggcaatcacgtaatgcataaaaattcaaacataatgtgatgaaagaacatatcacccgtctaaaaaattaaatatatactatagtgctgcaatacatccttaaattgtcctatattggtaagtcaaacgatacaacctgcattcttgggggataactgatgtttactggacggcggaaatactttaatttataggctactccagtgcatagtaagaatcataatttggtagcgcctagtaaaaagaaatcctcaaaaactaaacgctattctgatcgctatcatcaagaaatgaattgtaagtgagggctgtattctaactcatcctagcaggatttattgcctgcatcatcgacattctgttcgaagcggtgatccccatttggacaaattcaaggtttggattatctagcgcccttggagtctctttacgtgtttaggtgttcctgtaggaaaatcatcttattgtcgcgaatagaaggtacaaaaagacctcaaagttaccatatgcaccatggagatgaaacggtaaaagtaactgggaccaaagctgtccttccgggattcattattaccataatcattaggcatcaataatattctgtgcgatatgttgctcggcttattaacctcaatgaaacaatatgaccgcatatcgctacagtaaatctacgacgtttttactgattgattgaatcgcactttttaataattgtatgccccgatacataaaatgtcataatcgagaagcatatagtagtattgtagtatcctcaggatcggttggtagctttaatacgtgtaaatttttctcgtaattatcgagagtgtggagacgtccgtgtactggattcgtaagaattcaataccctgatgtccgtccgagtagatcgataaagtaagtagggatattcagatatttaatgtatttcctgtacactgtgacatctctgcaacgagattgttatactggcggcgcgtaggaaaaattcaaccagtctgtttgcagggatagttaaaattcattagagaccagagcaaataatgagcatccgaaatgtatccaaagcgatatacgcgcttacaaactctg

SEQ ID NO: 59 - DNA, 2000 bp, Artificial Sequence
  Synthetic
ttgatgtgcgaatataacattgatcatcagaggcaaggtgataggtattaaaacgttagcgtccacgctcctggttctataaaacttctttagatgctgctaagtccattgatttactgttttatagatacgagagtaaatatagtttaaattttttaagtttgaaatacgtgtagctatcgttgcgctaaggagagttgtctatgtactagtgatttcagtcggaaatagcagaaacatgaacctatcacatgactgtcgaatggaaaatttggagtctggaacattcagtatgagatatacattaatccatgactcagaggaattgacccactaatgttattcttagttgcaattccaggtatgtctagaatttgcaatcggttagccgttgtgtacttcgtatcaattttcaaacagaatacaaaaccacgctagttagccgaaattactcctaattgtcgtcactatgtaagagatttagaaaaaatagtatttggtactactaagataatcgctgtccactataaacttgtaggtagttagtcgagtgttctgcaagggtacattcatggaattcgcgagcaacgttcgcttctccccaaatattgatataaagacgatccattctatgtattttcgcactagtaaaatacctatctactcgacttacgctatagctcagggatctatttgtaggcatccacagctcagacgaaataatagatttacgaactgatagcggccctccatgcctgctaatcatgttcatacatccaaacaaatcgttttgttggtagacaacaacatagcgataatttcaactggttgaaatggttgtatagctgaatataaacgatcccaaaaaattcaagatggtggctgcaccggaacgacgttaatagcgtgaggaggtgttaaaagcaacaaaatcacacccgccgtcttctagggtaagcgggtgccagccgggtctactggataagtagatatttagcaaagaacctcagttatccattttctggttacgtgcacaattagttttgcatctgccggcttttgtctctggcacttgacaaacctagcaaaactcaactgaggggttaacacgctctaagattcctcttactagatgaggtattcatctgcgtatctgattctacgttataggctttttctctcgaatactaatgtctggactgatcaataagaattggctaattgcggaagtcaaaatagaaccaattatattcatacttctattattagttctaggatgattttcccgaccatcggtagtaggaggaggtgatgtaactcagtagtattatgctgagtgattgcacctctgattctattaatatggggggatgctgcttgcctcgtgggttagtgtccggatgaaaacccccctaacctattcacgtatagtatcccagtcaattgagtcagtgaccttaatcctaacaaaaaatacagaatgctgtgaatgacctcgttcttcttattgtgcacgatctgattcgaaaatgaacggtatagagtctgagcatcacgatataagagattcattctgtattatttacgaaaggcgtagcaccattcgatcagcgagcagaaccacggggcagtattgaatttccgtttttccgatttcaaaacggctagaaatggctgctggatgatagatgcccaactcacacggttgaacttgcttatcaattgtgcggttcatatcagacatagcagtctgcttggaagatattgagtaacttcagcattcaaacgcgcaaagctattgagttgcccctgatgctgtctatcgtgtattaagtgatcgtgggaattagacatacaactttacctcttctagcttgtttatagagcctcaccgaggtataaatcattaattacccaggagaccggttttgctattaccttgtaatgttcaaaaaagaagtggaacacagtgaaagcctcatttctcaagcaagtgagta

SEQ ID NO: 60 - DNA, 2000 bp, Artificial Sequence
  Synthetic
tgtagacatttgtcttcaatctaacctctttctcacgaaataagggcttgtattgttccttcgtttgtttaccgcacagaaacagcttcacttaacatacattgtaagtgtgtatttctcggggtacgtaacataacgaaacttaaagcaatcagacatacagtgccattccctacggtactgtctcagtatgttaatactactcatttgcaaaaggatgtacgcacttcatactacagctgctgacggtgtatatcaaacaattatattaacgctcgtaggatagttcacgtccgccatatctttgatttaggcttcaaaattcagaataatacgaaatagtctgtctactaggccaaagtcacttaagggctaagagtgtaatgagtaatcaaaataataatcgttgagtcgtcaattggagcatcagttatggcattaaaacatctagtgggtcgaaaggatcaggaaattatgtatgggtgagagtcgctgctacggtatcgcttttggattgagggctactacactcagtacccacagtgtgtgtattaataagaatcgcaatatgcgtccttttaagttttaaggtaccctacctttcatatctagtggaaatcatttacgcctatgcgacaaattagagacttttatttgtaaaacattggatgttggaatgaccctagatgcatgttaaatagcacgttcattagtggtacacgcctatcactaacgctatggaaaaatagaagaagccagaacaagtaaacctatggtgacaaataattacataaggaaatccctcataattagaataccataaaacgttagttgtactatccgtaatctaccttctagcgtggaatagttgagtgtattctagtcacgccccgttccataacgatacatgtaaaatttacagcgacgtttaggaaccctacaaggggagcagcagcgaggatagctgactagccttacaataagcacccatacttatgattgacatgatggtcatgcggcgttaccactccgctagcgttacttctttcgtcttgtaccggtttggcaatgcgatgcagcccaggtaccgtagagaaagtagcgatgtgtgaggtcgagtactttgtcagaaagcaagtcggattgcggtcccatttaccgcgacgtgcatttgtacagtatgaccgttttttaccacttactgatgaggccagactaataaacgatatttggtcacaggacaatattacggccaattatgaaataactgactggcctattgaatgactaggaatgtcaagtccagactctagctatttgggaggtttatatgtttggaccgacttgtgggagtttgacactacgagtaacaagattatccctttttatgctgcgctagttgacatggattgacgaggttattaatatccatgactaactcatcacagcttcccgagccgagacggattattttaatctcgttgatcgatatattaggtgacgtgagaagaagatgtgtcgtaatcagtaatagttaggatcaagaggttaaaagaagcgccttcttcacagattctcagtatctaccagcacagagttctcagtttctaacgtgttccgtatggatttgcgccactttctgaataagtcttatgagatatacttacctggtccagatgtagcagcgagttaagattataactgcggtttagcacgcagcgtttaaatacaaatactcttgactgttataacgttcaggattaggaacaggttcctcacggatatagaacccaattcacgtgcatgaggtattctatcttagggggaggaactgcgctggagcttgaaactgaccctctaggcgcttgctttcactgagatctattcaaactgacgtttagtaagaaatcataagacttatctacgccgccttataatttatgttattaaaacatgatcatgcgatcaattaggtaaatttctttgtgccttgcaatatg

SEQ ID NO: 61 - DNA, 2000 bp, Artificial Sequence
  Synthetic
cgaatatttatttttcctacgcacctacactatcgtgaagttcatggtatcaattatatgtcactagagccacaaatacgtacttaaatcatttacctcgactgaaggttgtaggcttggacatactcttgccaccattgtaacaaaggtagatcggttggacccgaaatttggtacttttaatctagaatcagcaatatcctacggaaaggcccaagagatgtctcaatggatgagagtgtaattacctaatttcagaaaagagagtttaacacaaataagaacagacgaatatcaataaagtgcacgtcgggcctaaatgagcccacagcctggatagattaagtgcgatacgtcgctaccaacgaacaaaagtatttggtattatgacatcggctccgacggtataggataggaataactcccaaacaatataatcttggatacgattaagtttgagtttgattgatcccatcaaacatttgttggtataaagttaatgtgtgatccagttagaattatatgaacatagtgttgtcacgattttgagacgaccgttaaacattatactgcggtggcatagcaagttcatctcctgacattagtcagcatttaatagtaagcaggagtactattaacacgctcctataatcggttgcctgttggggataatcagaacatgaaaaactccatattagaaaattacataatatagatcacgtgtatgaaacctaataccgcgaatataattacattatgattgcaatacatagggtagactcctagttaacgtaaaccaaataaccgactcgagaaacacaggactaacaattataatttataaactaagagtgctatactagttactgcctgatacctatgtttatttgcaagtcaaaagtttcaaatagcccttggcaagctacatgatgggtgattggaggtgggactaggagttccgtccttagtctgaataaagaacatgatgtgcaccgatttgtcgtctactcggacgttgtggcaagaataaaagtgaggtatagtaccgctagccgcagagatactgccttcatatgcgccgatactctattgttcataaacagcaatgaggcagagcacataatcttaattattaatttagttaacggcttcccaatttagcaatgaataaattttttgaggtgcatctgtgattaattcacccagaaacgctttcgcgaattacctgtcactatagatccttaatgaattatcttcgtcgtcggaacaattatcggactttattttgcctgttttatgtatcgagttaaataacgggaatcataattttatattacatctgttttgtatagcggatctcagtaggttacatcactgtcgtcggattcaacagcaacaacaccgttaatgaatatagctacactgcatgagtcccaacagcactggtccactagaaatatataattatacgaatactttgctatgttcatgacctgtcaaaggagaaatctagtaaagacccacggatatcgaagaacattgtagttctgactcggtttgaatgtccggtaactgcaggttcccgttatactgagcggtccgaaaatggcagtctaagtccccctacatgacgattgctatttattaggtctcagaatataacattagacacaagagcacaatagtcggagtatgcgttatcgagaccgtatatgagtcaatcgaacgtagatcgatcatagctaactaggtggtgtatcactgacgacttgacgatgttttatcgctgattagtttatgatcttgtaaagattggatgctacatattatggtaattttgctacttcccccaactataccaaatgactcactgtttatcaaaggtgactggataggcgctaggtatatcccggtgcgcaattattgccctggcgagccgaacatctcgaatatgtaaagacgaatactccctaattaccttttcgaggtaacaatgaata

SEQ ID NO: 62 - DNA, 2000 bp, Artificial Sequence
  Synthetic
atcgagttggtttctacgagtagctggcaagcgcacatagaacacacattgcatgtgagtggagcgattgcgagacgaaacaaccttccaaaagcccaacgattacagtgctagttatctatgggaacttattcccttagggccaaagtccctaggttattctatacgactcacaccgaagaggctgtaaattaacccgaatatagatgattagtcctttgtttgtcttagggatggcaccataataaaattgtcaaattagggtacaggactagttcgatttcttctatccgtcgtcctaggtttatatgtggccgtcaccactgtatcacatgccagctagcaacagtatgatgtatagcggcaaatcattcgtcgggggcatgcagaacgtcagttaactttaaagatgagactacgttttggtcacaatacaatgacttagactcatctcttaactcagacaatcacttttatacttagtgcaatgtgtcacagccacttaatggcctagctaatccttatagtcggtagctagcgagttatagaatcttgttgtggataatcctgctcaaccttgcctggaagtctaagaccagtactagaagttaggcgtcggagtctgtgatgctaaagttgttcggccaactaattaggggtgtacctccttgtctaatcctcttagaaattattcgagaagggtacagtacccctcacaaagagaatctaagttaccgtctgaagtctgagtgatccgttttgaggtaaacagctgttatacatacttacagcttagtctacatgacctactaagcgcttcgtgctccttaccgtcccagaatacccatggctcgcgtctcctgccgtacaatacgtagatttaatactcgtaatgtttacaaaaaatggctcagcgaatatgaatacgatatacagtaccatatttatggatacaaaatttgtggcatccgcctaatagggctttcctcagggcttactccacatactgttcaaccttctaggttcagtaaaagtggagaccacgatgcagtgtccttcttaatctggccttatttgtcgatcccttatctcgctaagattagtcacacgacaaagaggtcgttaatgacgtatctagccacaatcgacagtcttctggcgaagatatctacaagagtcgttgattcgtcacttttagccttgtaaaattgccctttgaataggtgacacccgaatggattggtactttcgtaattaaccgagactttggagaattgtctccggcgtttcatgtggcgaagaatagaggtgactttgatggcaccagaatctcactgacaattgctatagacctaatatcggatatttctgcaacttcctaatcgaaaaaatttctacaaaccagtcgcagccttgagtattcgcccttgacatagattcacaagattgagtcgcaaatggtcctatgataatggatgtgttattgctggaactttatcatgatgcaaagaggttataatattttgtgttagtagcacacttaatgcacgcagaatccttaatcaatcattagctgctaatgagaatcaaccgaccgtgttggtgttactggaattatattcagtatcgctctgatcttaaggccctcagcacctgaggtctaacgaaaatttttttaagcccattctcgcaaggccacaaccatcagtctctcgagaacgacattggacctcatatccaagcctccggttattcaccgatgtatttcttcgagtatctaaaatctgccaatacgattcaagagaagttagtatgcgggatcatgtagcgtacctttatatgaataaaacatacctggtagatggaaacttggtgacccgggagtacgtcattctggtactgatacttgagggtgaacatggtgcgtgattccagtatagcggtgaacctacgacaatatgtgcatggcattgcttatttggtgtatcgttttttgagaa

SEQ ID NO: 63 - DNA, 2000 bp, Artificial Sequence
  Synthetic
taactatatggtgtctgtttactacgattgcattaagatttctagcaatcttctccagtaactgcacttccccatattgtagaagcgacttatggagctaatctttcacttggtttaatgctaactgggatttgagcacgtaaaacttaactcggaccactttgttgacataattccgctgcttatatacccatattcatgtctacgattataaagttcttcgtatttggctaagcgtctctacctaggctcaagcctttttagccaatctgaacgctaaacgggtgctagcctagtgattatttaatgacgatttgagttcatggacgaaattacattattactgtctaaccggacaacgggcacgtcacaataagaagggtacagttgggatcgcagtttattcatgctgtatgccaattctactacctctcgtcatcttaattcatatatagctgaagggctagcaagtagtggatgactataatcgggatttagaagagttttttcctcgaacattagccttatgtgtctattttgttaaaattgacatgctaaacgatagctattagctggaggaataacataatgttgtaaaaggtaaccagctcatcacttcaggaatcttacttcctacgatggctgtcttttagtcgacgtaaagaaacccaaccaaggaatacttagacagacaggagatcatcctacaaagatagtcgatcttttatttagtccaacgcttaccaatgaatagggctgtctgagactcaaaatattggaccatgggtttcgcaaagcgcaaacggagaactatgatttcttgttgtggcagcgtatggtccccacgggtgactgtacaatcacggagacttttatcatataacgatagtacatttatctggataccggatccttcatttctcggaactctatacttactttaatttaatggcccgaaatctattatccttaaattacaccgccgtggactcggaatgaagatgagtccgcaaggcatactgttagatcggctgagatattgcctagtgcaatcgatcttttgatggtatttgtgtacattctaattcgaggcgaaactgtcaataaactaatgggaaaagcaagcatatcacgagaaatattctaggggataacattacgttttcggaacacaacaggttcgacataaatcttttatcatattatttgcttacaattatttagggcttccgcccatactcagtagttcaaatgatgcaaaggatgtggtgtctagtagatctcttaaatttctatcgaatggcgtagttacattgcagttatttttacatggcaaaatgatcaaatttgtacgcaatagcagtaacatattctctgtagtctatatctttatgattggagactgttaaaagctgatatgactaatcaagaaaatatcgaaatttgatctacgacttaacattttaactaagcagacatcataacgtttattcttcaacgggccgttactgctaaacattaatctaacgtaaatcggaactctgcagagtgcccgtctcttattttgtctgaattttagaatttacaaggagatgctcaagccgagttagaagaagagaaatataatgaatccaccgagtgtatgtttatacataaagaactatctttaggcgacgtgctagatcccactatgttcatgtgtaacgcatttattggtggaactctcgcaaaatcttacattatttcgccattacgtctatacaaaagctagatccgtgaagggtcataacctcctttaaaggcatgaaagaggttatctaacttatgattctataacatcgtcactggtggagtaaaaacatctgtgataaatacttgtgatactctctaacatccctgtaatatgatgatcataacgcttgcacctattaacttaaaagaaagttgtcttatggtgattcttaaataaaagtgcctgagccaccttgtgtaatttttaa

SEQ ID NO: 64 - DNA, 2000 bp, Artificial Sequence
  Synthetic
cacaatagtatagggacgtctattattgaaaattataccatgtggacatattctggatttgaatttattttttacgaacttactcgtctctttgtcgaactgatcgaaccatgataggcggtccatacgtgtagtgtgtgctagaagcatctgtacttgtattgaaaggaacaaagtcaaccatgctgttcaccaatttgatacgaaggaatgtcctatctaaccgggcttattttacaggctaagtaggtgaataatgacaggaaaaattcgaataaatcagaagagttttaagtaaggctcactggtcgaacggtgataatactggcggcaagttctatgtagcttattagataactcttcgggtgagagaaagagcttataaatgtggcgctgaaatccgatgccagctgtagccgagtcgcgtcatctcctaacggatcagttaacattatgcttactggacgtaaagtggcttgtctagctctcatgcgccttgtaaagctttttctcactgtgttcgattatagtgctctcagcctaccgttgcaaacaatgactagcgactgagatgacaacacgccacacatatcgagtggtaccgtattgggagggtagtggagagaccacccgatatggataacacgtacaagatgtggttaaagagccaatcacaaattgagcggcgatcgtgtcgacaatttttcattgtgtaagcatgcatgtatactagaaatagagtaatacttagcatatacgattaactcttggtgagatgagattctagctttaaaagaggggataccgatagagtaatacatgttcttttgagcaaatgggttgttcgccctgatccatgataacgactatttcatagctctaatttagatgcttgacccagtgtaaagatccgttttaactaacttagatgataatgagaaataaagtaattgactacttagtacactttaaatcctccagtcgatgtgtattgtcgctatatcgcaacccgatgttcacatacagggtcctgactttgggtataccttagtacgtaacaatctcactcacaatcaatccaagcgcggttactatgttacgacggggaagcaatacacagctaggcgtgcagtactgctcttagctctccgaaatctgatctagatgcccaaataattttgtttccaaagctagcgaggttttacgaccagtcatgacagattctgcagttgaagcatgtcacaggtaagcaaaagcgtggaacggatggagcgagtaatcaatagaacttactttacgagcggtgttacaaaattgggtataatgcactagccgacatcgatggtgtagtgaattggactggcaccctcaaggcctcgcccaactcagtctcgctagtttgctacctgcatcctatgaagctgtttttaaaaatatcgatttctagcggtagttaaactattaggaagggctaaaacaaagttaattatacttatgtgaacttacaatttatatattagaaagtgagtaagcatatctgaacaagcatcatcgtaatgaggtcggttcgaagtataaacttaagttaacgacatcttccaataccatcgaagtctactaagtaagttaggtgcttaatgatcattcatagtgtagcaagtccccgcaactagataaagtcaacgacttaggagtttagatagaattgtgtaccactagctcgctacaattggtttgtctagacttaatcccttacctgttgagaccgactctatttcggtaaaaatcggcaaaatacggtaacattgtctgcagtctgaacacagactagcttatatacatggatcaaccatcaggtgtgactatgttttattatatgaactgttaccatggcgcctacgacaatagtatatttccatttcggttaccagtttttgtctactttatccattaagtgatatatatacatgtgtccaacgttatatggacagcgttgtgca

SEQ ID NO: 65 - DNA, 2000 bp, Artificial Sequence
  Synthetic
taaaagaacggacatggcgcacaaaatgactatgaggcggttacttctgatgatcacaccctagttcttactcaggctattgtacaccctgccctctcaatatacccggaaatatgcatttatacggcaatcgatcttgaatcccagttcgagtctttacaaattccatcgtttactacgcaacgtcatgctaaataacaccttcccatatatgtagcgtgggcgggactattagagtcactttgtgctaaacagccggtaagtataatagtttactccggaaggtgtcaatatgtttagcgactgtattttggtactttatccctaaacttagctaatttacacatatagcagctggaggagcaaggtatcatttaatcttgcttaagaccctagtttgtacccctgtcgcacactaaacccaaaattgcgacattgagccacttaggccacattcgttaatctggtagttacagcacaatggctataatatacagatacgtctagaaaaaagttatttaatgcatagcttgcataatcgattctttaaaacagggtggggagctacgtatctaggattttattctacgtcatgataacgaatcttcctgaacgtactagatggcgactatcggagaatgatttagaacgccgggtgtgtcttgatgatataacaataagtaccacgaaaagaatgtaaataacttgatatcgactgtcacaatttgtttgtatcattgttcgtatcattatgctcctgctcgtgtcgcaattcccctttcaccttttggttctttatacacaatcatattatagacttatacggaatattggttgtaacttagagtaataccgattgaacccacatgtcgctgactgcgacgctacggcatcttaagccgatatatcgtcgtgacgtaactaggagtccgtaagcgaagagtagcatagcgatgatcgtttcagactcggagtattagagttaccatgctagccacatagaacggccttccgtaaccggtggcactcgttcgcagtgggaagcccaagttagaataaattgctaaatctgattctcccgtctggacttcgatcttcgagctagagtgccactacgggcactaacacattcaacgagtttcgtcgggtggctcgactatcggcacgagtgttgctctacgagaatacctgccttccttactgcgatttctctttacgctcttccactggtgccaagtggctgtatattactggtcgagtagggctcgctgattgtcgtgattcaaaaacgcaactctaaaatccatacctttgttgaatacctttattctcgttatcatagaggtgttcgggccctcactatcgatggcagatatagcttctccgctcgtactttcatatagatgttccccaacagctttaaagttagaatgatccactttcagggcatccagtaactcgagcaattatgtatgtaaccgatctttcgatgataggggatagtacaccttaacccttgtccccggtgaattgcggcgacaccatgcggtaggcgtatgtacggtgtgcccttaattaacatcgctactgtactacacggttaggtcgtttgaaaaggcagccatgaatgttaagatcttattttaaaattgatcatttacatttagctgctttgggggtaaatctactgatccaggtattaatctcttttgtataatgtaccaattgtagtaggttctctatgttcttaagtttcattgtcgataataaactaatcggcaaaggaagaaaactcaataacttgtattgtaccaaaaaagcgggggctatagttagatcggtgactcactttcttcgatataagggaaacccaccgtataacgacggtgatcttaagccttctcccaggttaacgtatagcctacaaatgaatgcattcaaaatgtcgtaagccttttacctggaaagcacaaacgatagcgcatttccttaaagtacct

SEQ ID NO: 66 - DNA, 2000 bp, Artificial Sequence
  Synthetic
acttgcacagaaatgacaaagacgtcgattcacgataaggcattccaataagtataacataatcgtgtttcggggcgcacaaaatagatacccaaaagagtgtcctttccactcgacagtagagctcatagttccgtgagattcttgcctcgtaactagtagactgtctatcgcaagaatatcacacccaatatttaacaacgctctgacgtagtagtggctacttgtgcgaataatctagtttctcatatttgcgattcaacttacggctaaacggcctcatagtttttccctattttgaacataagtcgctgttaagcagagtgatacttcccttatttaagtgtaagatgttaaacactaagctagaacacagtaagcccccgtatcttagacgtaatagccctgttagattaaaggattgcgatcgacataccaacagatgacattaaagcaagtatagcttcaattcccgccacggtaaacacctatcacgatacaaaggatagacttaccgagtaccgtagttagtaacctctaagctagtaaatcaaagttttcgctagttattcataagaacaaaattacaaaatgcgtatttacaactcatttacagtgatgagaccgattctaatccaatcggtgttagttttgcttatctgaaaatactgttagaaatgacgtggctgttaatcaatgtataacgtgcatgcgctgaatatcaatcatcagtatcgaggagttggcatacgcgggggctgttgttaaaaattgatccgaatcatctggtttactccactaatggattaagcctcctcaaggcagctgatgtgaaacccaaagatgtcaatttgatttcggtaattaattgaaatccctgtcctgagcagactataaacagataaccgtatggaaatctgattccttagacgttttcaaatctattcaagtaaatttttacgggaatcttaaacgatatcgttccgtgaagtaattcaaaaaacggtcttgatcttataattcacgtttgatactaatttagtcctccgctccctaatgattttttacgaaatggtccagtttattgtttttaaaactctttggaaaattcgtgtatgaggatgataaattgttcgatcaacgtttgtatacttagatctcaagcaagaactgtcagcgacctgtcgttaggtagtttgttgcctgccacctcgcgaccttaggaaaggaaggtaatctattccttaatacgtactatgtacaagagatgcaagaaaagggcaacatgagaacggttagtctctttgaccctcttactggttagtgaatatttttaccagctgctacgatgcaggatatctggccctttgactgttccatggacacgagcccgaaggatatttatttaatcgagagctgtatttagtatcttcataggacttgaaatcggataccgctgtaattgtggaacctcatgagacctcctaacaaaacaagtatcgacctgccctatctccgacatttactcaactctacccccaggttgacaatttaggatggtgtctatgggaaatatgattcgtaacgtgctgcctcaagaataggttatgaaaatatatatataaaattctatgatagttccttcgtctcactcaatactaagtcgttaagccaactagctcgggcgggctattagttgccatatgaggatccatgaatcaaacaaataatgcaattctgctaaaaagtgtgtatatagagcgtacacacaagaaacaaaactgaccgatccgacttaaccatttcaatataatgctgcacccttgtcctcaatagcttgcagggggcaattacgtttggagtctggttgtggtaatactcgactgtcctcggcgatatagaataattatagagtgtattatagcacaaattattaatagattccatagcctggcgttacatgaatattctcagttaaagcatttgaacgatcaagtggt

SEQ ID NO: 67 - DNA, 2000 bp, Artificial Sequence
  Synthetic
aggaacaatgttaatatcaagtcgggtccaaaaagatgtgtaaagtttgcgaaccgttgcgatctgtttctgtatcgtcttacactgtcagggcactaggactcactacgactcatatgtacattgtttagctcactccgagacgcttagtgaatcgttaataggttgatttgttattgaagctgtctgacttattatcttcttaaacgactttttacgtattgggagtcataggcgttttacagatatccgcgtcagtccacgacgtggtgctctatcggataggtacaatcaacaagaatgattattgctcatcttaatttactatgtgcgccgtttcaccccaaattcgctcaagctcagaccattgagggcggaataggattgaggggtagtgaggcgctgctgtattaggcaaccccggtggttcatttgaaaaaacaatcgcggaaacaactctaggcctaaggggaacaatcgctttgactatgagcttctatacctttgaatatacactttgcgtggagcttggcgcgactccttttgaggtaatgcgatcctacccattttgggttccctcttaattatattatcggcttttgtcaccatgatctcataatactgataagttacccctgatgttacgaccccgcagccgttagatattttatttaggaggacctacccaaggcctatgatcctttctctatatcacgaggattacagacaagagatgtgtaatccgcccaagttactctactcaaggttgcgcatattaggggagggcgtttgacagttgcagtatgccatcttggaaggcaacaataaacggtacacaactttacaaatattccataattgtttctacttttcattcattcattatgtatccctctatacttataaaacatgtacgacatgtcctgtagagcgggacctgttcccgctcatgacagacgagttatttgtctccgacgtatcatccatctttaaatattgaatagcagcagcatcaagtgtggataagtgcaagcactattaaatccgcgtgaactttcatatgacatgagaatcggactgtctgttatcgtaaataaacccgagataatgttaaaactattctaatgacttcatgaagcaggatcatctaaagttatcacaagaggtggtcttgagtcttgcaaacttcagaaaacatttacaaacgattcaaattagcctaaaccacttacttaaccactcatattccacaagttacggttctttagaatattaaggtgtaatgacccatcgagccttatagctcgaatcaagattaaaagaatattctaaatgaccataccggttacatgtgtgggcggagtcaaaagtttttctgactattaggtgcacaaaggtgttcagaacttaaccaaactcttagcacatttgattagctagtcagattaaggtctccactttcttttctgtggtagttcggtaaattgatgggcattaacaaacttaaggttgattacaatggggggttatcggatggttattgtaattgacccgtccatagatttgcttaaaaatcgcattttgaatacatatcctaacttccaagcattacacagcgctgcactatagagctaggatgactgtacaacctcggattatagcttctacgtaaggcgtggccgtggctggtataatagtggggtggagggagaattgacaaaaaaagtttatcatttaaatattagtaatggggttgtcgttctaggaccgtatttcgcgtactaagtcacatacccttatatattttccacagcaagtctatcattgcaagctgttaacttcattccggcggctgctgaaccagtatcagttggtccacagaagctaaagttagcaaagtaatacacgccaacctacttatatatgtatatcgtatagcttaattgagatgtcgtagccattacatgctgagccttatttttgaccgagaccaggtacac

SEQ ID NO: 68 - DNA, 2000 bp, Artificial Sequence
  Synthetic
ttggacgtcgaaattatttttgatatacgtgtaatgatagactaaaggcaaaaagaaggagtataagtctaagttcgaagaggcggatttggttatacgtcctgcacctcttgccagacattcttttaattcttgtgacctggacttgaagttcctttttgcgaccatttgtgggtttagtacgaaacccccataagcagttagcattaaaccatcaggtttgactcgccacattcgctatcgcaaatgctactaattcatcttaatctgacccccccgggaaggaagccatttaatagataatctgagtcgttccagagatgtacttctcagataaaccgtgaacactattacgacatatgctgaataaccagtatgtatggctgttgtcgactctcattcctatagtggagagaactgatacatacatattccctacacggatgttaaagagtcgcaggacctggtgaggcactggatcaacaagttgccaaactgagtgccagtggagctaatcacaccttcggctctgcgttacatgcgttagtgaaggtccttgaggtgtgccagcaaagattgttaacatataatctaagggattatatggtgtatatgggactgaaaacctagaggtctgtggggaaagaccgtacagtccctgaccatcacaataaaaaatagccaaaatagcgtgccattctaaaattttaatttttaatcaatcgcgactcctttggtttcatgctagttgattctatttaagaatccaagtgagttttaatcttaaccctaatgatttaaggttccagtaagcaaataaacgactcgccgtaaagcgaaattgatcgatacgtttcttgctttatttttgggtacagcaatccttcgaaatgttggcttcgtaattccctccagtaacttaaatcagttaatttgcattgtaagaaaacagcaagtgaatcatgtcgccgcttcagtaacttactgcaaaatgaaagcctaataaatagttacccatctatctaagtataaacgacttttgcttatgtccacccatgctaggctgtgaatcctcttacgtataacgtgctttgcgtgtactttcgaactttctaagtatcaatcgcaaatcgaagtaacttaccaccgctcgtaggaattgcatgttaaaaagggttaactcccttcgctttgtcgtttcccaacctgatgaaggaaggtgaaatacaacatatggaatgatatatatcacaaatacacacgactctggaccagtgcaaagtagttataaactcaaaacgcccccgacatacattaattctacttcgaaaaatatgttgccctaacgaaatggtttgcctaacagcggcaaaagatatgtcgactcgattgtatttaaatcgattattaagattgggatgagggccacgtagccgaaactgcaacataccgaaatgggcgttacaatgcattaattataatttattggcgctcagccttaattaacaatctaggcgtgctcatactgtgtactttaaagcaccatttacatgtcataacagattattgatgttacgtaaaattcatagtatacagtatcacctcgatcaaattcatatgtttttattttaaacaagagtactcctgtgtcgttctgaattactattagtcaggtgcgttaagctctgcagaacgataccgactatctgtgcatctacctgattcgaaaatgaaggcgattgggactctccactagttctgagttgtcctcctcgatttacaaaagataacttcagctggatgtttatcgaacgcacaaatcttaacaatggtttaagtagccgaatcagattcgccattcaaatctttgctctagtttcatcagtccgagttactctcaaaataacaacctaactcgtcttgcctacactggttctgggttttatatttagagacataatcacgaaacttcatgcactatagaaggcaccatgctgttc

SEQ ID NO: 69 - DNA, 2000 bp, Artificial Sequence
  Synthetic
tgagcttcgctttttccagagtcgctgactaaagtgaagtgtctagtcgttgtccatgcgatatcggggtccatcaactagaattcatttacggtacgcgttgtcatgccttatatttagcaataagactaacggaagctcctctggagggaaagtaagaacgtccccccgggaacatacctaaaataaaggtgcatgaaccatcacggagtggagacgcaaaagatcaattagtacaaatcagcaggagacatgcaaagaccgcgcccctttctttttataccatcttaatagcctttactgatcgtgtatgttttcatcgtgcacctaattatggaaattctatgaagcttttgctcctaatcgtttagtaatgctctcggatgccacgttatcttactgagaagcccgtgaccaaagcatggtgacaatagaaccaatatatatgaaaataccgggttcgtctgaagactgtgtagtaacaaaggtattcttgtgaattcacgtttttaatctcatctactatcggatatgacaacaaactctgattagggtaatataaaatttaccgttcggcctaattaaaggacaaccggtatgtaaaacagcaacatcacctagcacgaaatttacctatgagtgtggaattcgttagcgctgtcgacgtgcataacctacgggttgttgcatacgggtcagtgggataatgttgactcggtccttagtaaagactagctcttcttattcttgcgcttgtaactgacaagtcgagttcacgtgggcgcagtaaagtcgggaagacggtaatcgcaaaagttcggtaaaactaacagtttttaacgagtccgtaagttcaagggcctaaatagctggaggattttaacgtctaaacattcgggacacagtgtatgacccgcataaaaggttcaaagaaataatacttagagccgtcgttcggatcttatatgtttgaatgaacccttaatcaccctataacatgaagctacgacacattaatcagatcaaaacctacttagagctcgtccgatactacaacttgaaatcttccaccaaaactaaagggtccattatgtcaaaataccatttctatttatattttaaccatcaattcgcctatacccctaatcagcattaatctcgcttaaagatggtagagttaaatacaacgcagagcttttatactaccagtgatggatcacaggattgcgtttcaaaaggtgatagcaattaccaatgacctttgacagtaatgttacatcctaaccggattatttggaataccctctatttgctttctgtttagccgacgcctgtaattgtctacctgcgtgcgttgtgatgccggtccgctcgatttaagcactccgatatctcatgtaggtgtggactttggacaaggggaaataactctcaatgacaatcgtactgcttatgttaggcaatgctggcatatgcaactctgaggctaactaagttagtcttgtccgtgatctcagaacagtaactatttagttgcttgcgagtatatttcggtagagacgtatcttctactaaacacggttaaatattttttggttatctctcgcccggtctagtagtgccataacgtttacgaggtcatataactgtcatacattgcaaggcgctttatctcaattgtgaacaagtaattatagccatgatacaatttttggacggaacttgttttatctaaatcgaaagaacctacattgcctcggcatagacctcggaagcagctagttcactagctgcttcatgatggtccaagcttgtgaaagattcacataaaatcaacctccgtgggagtctccgatggacgaagctgtgtgactggatattatctcatgattgcgtcacccttaacatgtgtgaggtagagctaactatagaaataccagtcgagttagcgacataatgcgaattgatccgcctgtcaattcctccttatacgcgccgtt

SEQ ID NO: 70 - DNA, 2000 bp, Artificial Sequence
  Synthetic
attgtccattcttgtatttgaatcactccctaatgaaccaaactctctaagcccattcttgtagtatttaacacacatgacaacggtccaattttcatgtatagtcggagtaacgcgatatactgaatcttctgacttatcagacatataagatgtaaaaacagcggatcaaaagtgttctctgctgggtgaaaaatgacaattaagcgtggtattatctctgtaaataacacagggatttatatgtaaggatcgcgccctcatacattcattaattctcactcagacttccctccttcgggctacgttagattgaaatgaaaataacatgttgtaatcattaaatagtacatactgagtttttaaagtcgaatactacaaaaaatatcatactttttttaccagttcagtattggagtcgacacatgatctaacataacagaagacatagcgatggggattatcgacctttttatgggtagtaacaggtggttgccggatgcactagcatgatcaggtctcctactcacacagtccttctgactgttaggttgtctttgcttataaaaatactcggattattgcgccacaattatttgatcaacgagcttcttggagagaataaaaatattacacttcggatagataatacaggttaggttctcctatgaatttgaagatcccatgttcgttaccgtccaagagccacggcttgcttgctcgaaattaaagtgggcattcgcgcgggatgggaagtaccctcagtcttgacaattcccatcgtcaatattagaacggtggattcgccatcaccaggaaacgtattgctgatgatgatttcaatactgaagtcgtacacttctcacccggaaacgttaaaaggacgataatgacttaattgagatcatcgaggtacgagcccatgccttaggtcgcttcgtaggggtcctccttaaaggagactgtttcttacatgatttgttacttcgttgaaaataaatcatggatcgacgtcaccaattactggggtacctgagtatatagcgtagaacgtgaaagtgattacacctgtataggaaatgatgagctcggggaaccataatgaattatagtgtaaagataaaaaacttgccccgtgccacgagaaggaatgtagcagacaatcatggggacattgtaacttacccagactttaatttcgttttcactataccactcaattatgatgtgacattctggaattgatagcgtatgttgcagccttctaaactcaacactgagctccttaagggttattatggttatatttgagactataatataatccgagttcggtcgaagtgagtaatctttggagggtttaggggggcagaattcactataagcagcagagattttcttagaaagagccgggtcccgttccaataagccctaccggacgtttataatcattggtgcatcagtgaggccttctgttcatcttctattctgctgtacccttcttgcaccaacgcgttggatccttgtatcgagtcactgccaggtttgtggattttttgcagcccaccctacgttatatcttaacaatcggataattaaaccaagctatcgaatgctatgagctaccacagattatcatcgattgttttccctatcattacgatccctgacggactacttagtatgtccttttcttaatattcgttaagaactggagtacaggctgattacacaaccagtaggattaggattaaatagagaaatgtatccggaaaagcggagttactgtttgggtctttaaccgcgaatcgcggttttttttctaatatgcagtgatcctttatttggttactgtacatctgctgaacacgctatgtggatctcccacagttgcaagtgcaaaatattaataaattaatcacaatacagtacagctagatttcatactaaatgctgatttttgaccgcaccctcgagagtaattcaatgacggccatgta

SEQ ID NO: 71 - DNA, 2000 bp, Artificial Sequence
  Synthetic
aatcagaatgagcagatgtaaaacatatttatgtaagcaggttatcccgtatggcactcgttgctctaagtagatgtttttgtctcgggtaacttatgtccccatcctcagagtgtatttacttttatttaacccgacggtgagaacatacaacgggtcaacaagacaatacgaccattatactgctaaactctcttcctcaggtgctatatgagttacgacacaatttttgatgttaaagtcgaccctagctgctaactgaacttctgggacttaaaactaccagaaaggatgaagaattagtttggtcaataactatatacgaaacgccctgaaggaagtcgtattaaatttggagtgcataagacatggtgagcgaaaactaacacctacctcttagatacagattacttttagttatcttctggtctatcgttgatcattctaagtttattcagcactagagacttttggaatacgactgccaaagctagtataggattatctaaagatcattattattaacggataatgcgaaatttgctagatcgtatatactattaatgcagcaacttaactaaagatatatttacagtggggcttatgcaaccggtgagccctcggttctttatgattcgtcaagtaaagttgcacaacgttcacgatttaatcttattctttgatcttgggctgatgtatcctcattatttatgatagaaaattgattggtgcatttgattcgcccgatactagacccacagctgttgttcgatcccgtatacaatgagagcatgttcagatcaacagtaggtgtaacatcttatgttccgagccttctagtaaccaacgaacacctggcaaatgaatttgccatctttccgctgtacgaataggggtaatgtgcccttgatttaaaatgttatcgataggggaactacagatactgagaactcctgaaacgacgttaacaaacctcctgcaaaacttgcactctttgaacgaggttgcctagtttccagaagtaggttcttgtcacttgaatttcgatggaattctccttatctatccagtgacgaggaagaagaaatgggtttttacaaggactaagtgtttagacagaaaaactaatctttcagtaaaggtgagaagtgattttgcagagggagattgtgttacgaggatagtactgacgtttatatgagaaatagttatcgataatgtgcgtgtctttaccaagggactgaccaactgatgtggaaatttaactcttcatgatcacataatttcaatacgttaacagttagaagcggtgatctttacaaagtagacaatgagttattgtcccatagcaatgcctaatgtcgagcgtgcttcaaacaattgaatggcgttattttttgatccttaggaaacaaaaaccagcaacgtaacttattcttgtatcttcatgtaatcacattaccggtatagagatggttttacatatacgcacgttactttgagatagcgaagcatacgaatatacacgatacaatgtcagaaggataaaatcactatggcctcactcggtgcatttgatttcaaaggcttaatgtagctctgttcgcactcgtggatatagttggagccagatagactaggaagatgtttgtttagatagtatcctcgttcgtgcataatatccttgagatagtataggtcgaatctccacagcagcaagattctccgtgagcattgccactctttcagtagtaagcctaagtaattcattaagcgtaattagagacttattttccatatctgcgcgtcgagtttcttctgcagccctagttaggagacatacgggacgcttgcgtttttatcgtagattcacttagtacagggaagataaacatgagaggaaatccgacacctaacaatactttcaaactgaggggctggattgtacttaccttcacatcatcgaagtcaattcttcaccttcacaagctctttcttcg

SEQ ID NO: 72 - DNA, 2000 bp, Artificial Sequence
  Synthetic
atttacacccatgccgaacataaataaacaaacacaaaaggatgagaggaataatgggttaactaaggggagtcgaatcgtattgatacttatgaatggctatgttacactcaggttgtactggatttcgtttgcgctacagcttagacctttcgctaaagatacacgccgcagtgtctgaaacagacgcacatttaaaccgctgggctgttaacgctcattctcgctgaactagtctgtcatttatcagtgacatcagcttatctccaatcctcataagaccgtcgacaggaaccctcaattccactcgtaacagtcccacgctgggttgcgtagtctgttgtaagaattcattcatggttgaaatggggctgatgactatgaggcggcatctattggtatggtttagtagacgatcagaggaagtctgtatagtcagggctcaatatgtatccacgtagtaatgttgcctgctaccgacacgatttagacaacgtcagcgtaattacgaacacgacctcggttccacgtgtcatcgtctagatggtccctttgttcgtaggcctccaagacctcagtaatatctaattcgagcttcaagtttgctagacgttgacttgacgtagcagataaatcgcactgtaatggaatgatacctgaatcccgttaacttccagcatggcacatacgatttttaaattacgcttaagataaagaagcagtgcggtctaatccaaagtgcacaagcatatcaaaactcaggtctggtttgtacgattatttggagcagattttcaagatagttatgccaatctctccataaccatatacagtgacggggaccctctatgatacgtcatctccgggacctactttgacgctggagtcttacagatggtgggaccatttgtgcttaagctacttttagtgcggtaggagccctccacaatatgattcaaacctaaagaagctaggagccctctcgaccctggtacttggcattggcttaaatttcacgtatacgccatagcagattagtttaatctccgattttcaaaatactagatagggagagttctataccacattaactcgccccgatgggagaacgcacaagagttagttttcgacgccgcgtaaaacaattcaacatggccctcgagtctgctactgtagtgcatgaaagctttcctagttgggctagtagcccaagattctggaaaaattcaagttagtcgacagatgtttccgccttacgagtaatttaaagaggttaccccgagaccgcaaagagtttagtgcatcttatgtgcattgtgttgttcgtcagggggctttgcacctaaacggtcttacgtacaagctcagttcgtggatacatgaaagtcttggagtcaagacctacaaatcgacgcgattctaagtctaatgtatccttacttcgggcgtattgtgatagtatcataacggttaagacagtttaggataaaccgcagagacaaaaaatctcgttcgtgtaactgagtatatagtgtacacttgtgcccgcaaatgcatattattgatcgagtaatttaacgtgtgcctccttggtagagggtttccctaacatactccttttcctgattacctcagtctcctgcttcaaccggtctccataagtgagaggttgtgtgtaccgcactttagaagagtagaggtttggcaaattttgggagcattagactagtcgaatttcatacttcttagtcgtctgggagaacgtaagacctgattaaacgcatgatacacgaagtcattcagttcttcagttaagaggttgcatcaaatagcactagcttaaatgtaaatcgtcttaagtccaactattatgcggcacttgatcaccatttcactcacctcatcactacgcttgatagtatgatctcatcgtgatggtacccagttgagatcagcgaggatctcctcataaatttacacattgttaaaaggtcccgcgc

SEQ ID NO: 73 - DNA, 2000 bp, Artificial Sequence
  Synthetic
tagatctgctttgtgaatgccgaatttcagattgactgtccgcgcgctagctcattatgacccggcagttgaaatcgtatagggttggacccaactactaacggaactcaaccactcgccctgtacgagatcacagggaacgtcggctaaggaggttatggtggccttaccttagcactatataaagtgcgttcgaaacctcagtgattccccgatagtatgatttttaagttctaagattaaatttgatacatcagttggtcctagagttagtgctactaagcttaaatcaaccaaaattttacccgttctattcagaaggaaactatagtggtagcaagtgtgacagtaggtatagacttaaatagttacggcgaaatagaaagattacgacgttcagccttgtgtatcgaatttgtgactttagaggcacacagagtaatggacctatcatctacgtcctgtcagagtatcatgtgcatgattcgacagaaatctcaataataacccaaatcgggctctcttgcattgaataattcatcatcaacatgaggtaatagcaaaatgcctttacttcagttgattagggtgatggccgatcacctatgtatttgaacatatattgtatatccggtcggaatatggcatccttagccgtcgtgcgccggctttcggaatttgatctgtctctgtttagacgcgtaacctcaattcgccgcaaactagatcactattctaataatctcactaggaatctattcgacatgcgatctttgattataggattcagaatctaagaaattgctacgatggggtgtcatagcgatgtctatttgagtttctatagtgaattggccatttgttttggcatcatagatcgctgacacaatcattgtgtctttcatcgatctggagtacagttagaagagaagcgagggctggtaacatgcttatagattcttatacttactaccttagggtacactaacaatatttgacattataggtcgaccaaaaagatttctctatcaggtttagagacaaagtcgtcgacatatttctgtttgaactcttgaggatgcacgaaagtgtctatcggggtatcagtgagaaggcgtggcaagcattctctaggtgaattccaccctttttagtcctcgttagtaccccgtagaccgcggaacatcgagaagttattcgtaaacgtgtctatctgttctatgttaggagtaggtcattgaacaaattgagctttcaaatagattctagaatgtagcgcgtaagtatgtcccgatagcggttttcagtgtattagttgcatctaatgtaattgagatgaagaaaaccttggtcgaagagacatgcctaaagaagaaggctaagtgaaggcctttatatcacgtggttcatagcccattatataaaaatttatattggagatgtcccattggtattgatagatggttggtagctgtcagcagtgcgccctaggtaaaccagaagactccttaacagatcggtataattattcgaggtttccggctctagcattcagacatggaaggttctttctaagcggatatattgctcgaagcccgtgaacctttagaatcaacctttattatctctaaccatcttttttacgtttcacctttaacttacgcgaatcgattcacgactgccgaagtacaaacgatgactcagtgttggttttcgctacaacattgagctcagctctatagcgcggactacaagttctgcgtagattttgccaaaaaaagttgcgggtagccttattcatttaacgtatgactgggaggcgctcaaatctctcactgcacctattcgcagacgcaaattatggcgtcgaccccaaactttcaggtaaatagctcacaagattgaccattggcaagtttgaactagtgtcgtaacgtcctgaacaaatgtttttctagccgctcctgctaaccttatggacattttcctcttcacccctg

SEQ ID NO: 74 - DNA, 2000 bp, Artificial Sequence
  Synthetic
aaactacagaagaacccaaaggctactcactccctttgctgtgttcagctcgctggctcgtcaagataacggactcatgtctgtgggcaaagcaatttattacagctatacctttgtggaaaagtctccttgtaaaattgttagcaatattgtttcgagttatatcgaatttaaggtttattgttattcgtgaccataaggagctaacatgatgcggtttaatgcgtatggaaaagcgatagtgtttttagtgagggaatgtagaagacctcgtttcaacccttaccatacccgagggtgtcttaatctgttattaaataaagagcagcaaaataaaaaaaaaatgcagtgtctatcaaattcccaaatttggctacgtcgttcactaccaattttcaaaataataagaagaagtatatggatccagtctgattgtctttccgatcagcaatataaagcaccaacgtcttataagagctaaatagtgatgattccatgcagtataattcaattcccctaaagctactgtcgataaacttcatataacatatgtacttggaccgtttggtttggacttgacaggctttaagcagtctgcatcatgagcctccttctagatgtgcaagcattccccagaggcggttcgcttcagcgtggtaaggaatgatctctgggtcggaggtagtgcagaatgaccacttatcctatctagtggtttactttatctaaaacaacaggggactagatcttattatacggccaaaactgaaatgaagatcatctcatgaatattctcttaacatgagaaatttccgttgtcaatttttaaatggattaatgtcataaaatctgggatatggcgagcttaacacaatgcccctagtttacgttaagaaacatttgatacatcaacaaaacgtaggatccgccccggttttttggaatccacttctagaagcaggagcgggtcgctgtatttaagtcataaaggacgtcgttttacgaacaagaccgtgtatgaatctggactgttacaacggcccatccccaccactagttatactagtcaccgaataatctgaactattttactagaaagtctagaaattcatcctttgacataaatggattggaattaaaaaaagaatttcaaatataatcatataaaagtggatgcaccagagctcatgcgacgtcattctacgagcgatttatagcttataccaataaaccccgcgtgtattaacggtccagtcaaaaatactatgataccgaacaaggtttatcgacttgtcccgttgaaatcctagatgaagtttataaccaaatggcgcccctttagtgacgctgtaaacgcagatttatcaaacaggaaacatttctgattaaccagaagtatgcgtagtgaaggtatatcgcgcagtaacattcaggtgcttcggggattcaaaaacgtgttgctggtatagctcgcctgttttatcgaatgtagtctcaaaatctagccgagtttatcaactggtcgacgctggaagtctgcacttgaacatcgttcacatgtaagccagagataatggcctcagcatcgtcttattgctaatctcacgctgctttgtcgcgacgtactctctgcattaccaaatgggattagtttaatttcgttctctgggtgaccttgtgcacgctatgtgggtttgtattagttgattaaagagtccctttgaagatggcttcactcaccacatgactacacttcctatcgaggtaaggaaacgttttcttgtgcaaacaccccagacttaccaagtttaaagttttgtataatattaagaatttatctaacactgagacaccatacacagcttccgtaccctattggtccacaatataagacgttagatattgccaataaatgcttcattcggttttttgttagacaattggaaaatcttatacataacatataaacgtttcgcatccctggttccttccgataggtc

SEQ ID NO: 75 - DNA, 2000 bp, Artificial Sequence
  Synthetic
tcgttttatcacgttttaacattgaatctttagtgcaaccaagagccacttctcctgggttataatcatcatctatttagcataccaacgcgtttggctgcctcggtttgtatatagtcgtaaaagcctccggtttatgaggtgatggaaattagttggatacttgaatagataatatcccatgcggtattcacccactgaatcacatcgcctgatgatccttgctgtttgcgggagagctcttctaatgatttttgcaaatgctgtgcatccctaatagtcttttacagggcaaagtacagggattgacagcccccgaatgtctacagccgacaaaccgaaagtcttctaccccgaggtagctgaaggtgcatagacgtagacatgttgactaatctcatcttgtctactatcttgtacacaaaatcaaaattacaattatatggaaggcatgggatgagtgatcgttaattagacaggggcgtctttggcaatgcattctcttatgataaaaggttgaccagattactgctcatgacttagtgtccaccggcccaacaattaataattaagagactcaaccgacatacgttaatacccaataatgccccaatacccagacttttacagggttattcgtgaacatgagtccctcgacatcttcccagattttaatccccatattactagtttgtaacagattggttatgggactgattagaacagggaatttcagctggaaatcactactaacttattgctagtttgccgatctaagaagagtctttgctaattgattttaaagagatattctgaacacgtcaatatccaaattttatccgcaccattctgacgtaatgacgcctagagaacgagttggtggcagtctatcgcttctgtttattttaaccttcaaaatatgataaggccccagttataaactattttttacggcaacttcggattaagtgttctatacgccaaaactattgatttacttaacatttcatcccgagaagctccgtcttatcaagtacgagatgatcccctattagaaaaaccacggctagtatcaacgacatgcgttacacacacgcctcagtgggggccgtcacacatagttcaaatattgatactgctcgtctcgatatgtgttcaatgtcggcaatcaagcagtgtcggaactgaacccgcactacgggctcgtaaacgacccaaaatcccctaatcaatcattgtagtaatggtagcaacttgtatgtcctgtcaacgcaacaccctcctggtgaattattctattagaactactaaaaaataaacccgaggtccagctctatcgtacacgacacgaaaacgtatcaaggtacagttcgatagccgtacttattatggtgactagcgccatatacaaggtcataagggaccttgttagcggtgtgttcacttcatcgtcagcgactcgttcgactgtcatttcaatgaaatctttaatgagtttaatagagtaggaagggacagtaagatattttatgaataatgtcgtacgtaggatttttttcaaatgatgactatcacagtacggcatacggaaaattcagtagggaattagatcaagtgtaaaattactggtatactagcgtatacctagtacgatgataattaacaatcacccccagcatgatgtgagaatagtaaagtatccatatttacaactaaaaagctcggaagctgaaatcccaaaccgcttgaacagctctcgaataataccggtgtttatcatcggaaggacagcgcctcaggattttcggcaaatcatagctcttatcttcgatctaagcgtttgatgaatattagaatcggactgagatataaagaatagtgatatatgtcggaaaacgacgatgtcattttagactatgatcttaagacggagaaagctaccatcataacaccgacttgtcctgccattgtattactggctttccatcgtgagggatagc

SEQ ID NO: 76 - DNA, 2000 bp, Artificial Sequence
  Synthetic
attatgatcccaggcttcgttgagtctaatagctatccgactaatcaacttctcaggcatgtctcgactccgatcctggtggccttaaatttcttaggtgcacggaattgtgtgtacctggtatgtagagactataacgactcacttcttgccaattaggattcaaaactccctacttgagcaacgtgttcccccgcattatccatatcacaacagttgaatttttctaacgtcttctcctcaaaccggagggaagtgtgaatgtactgttgtccggccatgcctgaggtattttgattctagttagtaattacattaggaactcacttcgtcaactcaaacacgttgacaaatgtgcagttgggtaatacatgccgtgcaaagcatgtatgaccgtggtctactagatggcttcgcgatttactgttttgcgatataggcgtcggaataaacttcagcaggtgcggatgctgatctggcgccgtcatttataaagatatggctacgacttagctcgtgagatcgagacaaaatcaagatcttatcgtcttccacaaaaagtaccctcaatcggatattcggaccgtaaaaaagagcatggcgcttgattatcgtagctagcgcccaaggaacaattgtattattcagattaaaccccggattggacctattttcatcctagtagaaacggtgacgacgcgacttccgaaaactccaggaacagtgcggtctacccaggttgtagtagatgcccgttttctcagggcaaccagggcatcatacgttaacttaatcggttttaaccgcgaagttcgatacggactgatttaataataaacgcgaacaacctagtaatatcataaattgcggcgtgtacttcagaaatggtaactaaatgtcagacttcttgaaaaggaacaagcgcgctttctcaagtttgttgagtctcatcataatgggggaactccgtacatggtccgatggactcgatatccgaaggcgataataattatccccgtgttctacgctatttacgaactattaataatgatcggtcatgtcggtggtttattccattcctttatctccgataagtacgttaccatgggattacgcaacagctagattttcaaatgatcgggtcgaatccggcctaaacgaaacgtcgctagcgattgagaacggatgtacagatctctcgaatacatgagatgcgcgtaatcatagtgtacgatagaacctcatgttatcaacaggtgctatcttagtaaaatacatagtcatattctttacacgcgtaaagattctttgagccagcgaacatggaaatgggcgttggtgtgtttctccccggctttcgtaatagtcgccaccatccgcttgggtgctgattcgatcagttctaaccaaggagcctgacagtcttcgatttttgtgtattcctgtagaatatggcaccataattcagcgggaaaaaattgtcaactcagcagtgtctattaagagattactctcgcttttggactggtacagcctttacctagtaatatagacggacaaaaattttgtgagtcagacggcatatcctgaaaacaaatacaagtgtagtctacgttttagaatagactgagtggcgtcggtagaagttactgctcgagttattgtaaaattcttgccaagaacgaagttactccatatggaaaagatgactcaatcgagtcttactagattatttccgaagtcttaaacgtttagacctaacttagtcgaaagttgagctccagaagtcatctctcccagtttatcaatagtgggtggaacaaattcatcggctgttgaccttattgcatccacctcgttggagttatcttgccatgtatcctcaagtgttccgacctggaagtatgtagaaacccctttgaaatatctatcacaaagcaatatcttatattatcttcgtagtttttagaattatatctatttaagggcacaaagtctag

SEQ ID NO: 77 - DNA, 2000 bp, Artificial Sequence
  Synthetic
ttaacaataaatgattaggttgtgcttgcctcctaattttgtttaaaaagttgttcttctgctgactagtttgattctactcatttctgtagtaccggttcggcgtactttttttagaggaaaatactaatgtgcggaggagggcttaagaaaactgcagatcactggatgagcaggaaaaccgaaggacgtgcacgaaaatcggacttgctgttgtgactatacgcaggctagaatcaataccgtcggtgctcgtgcctcagccgtatcagatatgattcttgagcgatgttatcgttggatcaaatagttcttttcgtggaaaggtatggttagatatccggggcctcttaatattggtttcgactagatctgacagagtcgggtcaaagctaacgctgtcgctaatgatgacagtgtcaatctggttaagtatactctggagttattagtcgatctctctcagtgtttcttaaggtgttctcagctggccgggttgtgcgcttgtgagggagcgatagcagtttgtgctcggtctacgcagtagatcgttcacaacttagtcagaccaatttatattcctatgcctaagaaatagtagatcatctaaatgtagttgccgatcaactcaaaaatcatgagcagtgataaacgctagtacggagctagcatatgcgcctgccgatagattgcatagaaccacagaatctctaaatttctggcactgactttaccttacttgtctactgatcatttagttctaaggcgggtcccagcatatactgagtaaaggaaattgcaacggtccaacaaagaatcaataagtaaatagaactcatcaatctccatggttttttaccctgtggtatgagagcttcgagacagtacaaatacattctacgagtgcatttattaaacacacggaccctatacaaattaatagcatcactagctcgaaacctattacagcctgaacgtttcgaacgcacttcggtatacagtgtactcgcgcgcgtgttgaaccgaaggtgctagccgaattagttggattcgtatatatgtgggatcccgatttccaagtccttgctggtttaacacacggatattagttgctattattagcgtgtttgaaaaccatgtcagagttaacgaccggctaaaaagccgacttataaaaagccgagtggtttggcaaccttctactggtcttggaattaacttctgaataaatacaaacatgaaaagagtgaactgctagactgcacctgtggaatgatccataacagttaaattactccgccgagtccattttgctgacggtggattatcctaactgaagagcgtacagcgattctgtccaaccgttgaaatcagtaattttctatacctactatcgtttgaccaaactcagggaagcatacctaaatatcatcaaggcgagaaacttttagacccatagttgtattatagtctaatttcaatgcacattctgttcaggcacagactgatattgaaagaggcccgcgactttgaaggtgggctaaatttatgcaataatggcacaccaatcaacacagtctagaacttaccaaaccaagcctagattcacctatctatttttgatccgactgtataacgtattgtaatacctcaagacataagacactcataacaatttaactttctcttattaggaggctcctctatgggattcgtcgtcgagttaaatgatttgaggttttatgtggactccgagcacgcccggtaagaatttctaggacttaggatacaatgcaactcagtggagtatgttcccccgtgtgatctatatgatagctgagtacgacaataggcatgcgattcagactatccgcttttaattaccaatgaatgtcacgacggagaacgttatgaaaggttttctctagcacgccctatcgctcttatatgcgaaatacattcctgcttgtgaatggccgggattgcttacacattagcct

SEQ ID NO: 78 - DNA, 2000 bp, Artificial Sequence
  Synthetic
cttaagatttcagctagaatggttctggcgcgcctaagaaactaggttaagtcttcttttgcgcgttaaataaaaattttgtcggtagttcttaaatggtgcacgaagttgactgcatatatatatgaagcacctaagagctctatccccccttaaatgtcaagattggctaatataccaccccatacacatgattaacccggttaccttcgacaggtttggatctttaaatacaattagttgatcttcgctctggcagagctcgggttcgttcgtagtgtataaaatatctctacttgcaattatcgtttaacccctgcaagagcgtctattggtcttgctgttttcttacagttgtatgctcgccatgtataggcaggtaaacagactttgacaagggtgggcgagtcgcgtagaacctttccatgaaggcatttatttttgattatctctgatacctgggtgtgtataattggatgcaacgtcgcttgctaagacattcgagctcgaaattctaggattttgtctataccctttagaatcttcacttctataaatgactaaaaacatgggaaatgacaaattagcaagcggcgcttttttgaatcaatcactagatatatttctaaaacttagcaatgctttcatgaaaaccactaattttaattacatatttgtaaataacccgcatcaaacgcaagttgatgtcgcatcatatatatctccatagtcatttctattcaactggcatgttcggttaatcaaacaaacctgacaacattattggtctcatcaaaatttgctctattggcatccagaagattgaattttgagtgaccagtaatattaccctctgggactacttgtatcttttgtaaaagacgtataattgtagggaaaatttgaagttgtaaactagaacaatgaaataaatcacaagcctcttaaatttccgagtgtgtttaatagctgtccgaagaataaatatccagggaggatctgatctctaaaaaggaaactttcctaggtgcaattcatgggacaatagtctttaccatcatttggatcggaatctttaaagatttaacgtaaaactgtagatgggtgaagcaaccactggtgtcaggattgttgtaataacctacaatacgaaaacacatggaaatatttttttcacgagctatacacgtagttatacgtatgaaaacaaacaggactcaaataatctatagaggaatttataggttcttcgtgaacgtttcgagagcatagacatgattacaggctgcagatgattgctctagggacactggatacgtctgtctcagtatattaagaggcattaacttatagagctggtttgagttcctcatgagagagaatatatatttgcacaatgatactcaaaaacttaccgctctgcacaatccgcacatcgcgatcatacgcgccgttaaagttatcatccaatatactcataaatggtgtaacctagctcctaccacaaactgagtaccgggatcgctatccacatcgctgaaacaatgggaaaagaaaggtttccttcgagtcacgcactgactagatctacaatacttatgctctagaacgcgtgatatttctatgtaaagtaaagcatgctactaaggtacatctaattttacgaaaccgtatactactactcgccattggtatactttagactttgtaagtaaaaaacgagtagggcctcaaggacatagtcactgcttatacagcgaaacgaagctgctaacaaagctcagaccggtattgctgttagtatattcttgttagaagcgtacatcggttgggccgtatggtccgattaccttaagaatagttgactaggatcgtctctaaggtcgtacttacccacctagcagctgatatcttcgatgcctatatctgtataggtagagattcattctcagcgcattgccgcggtagatcctatgtagattatttagcatagttaatta

SEQ ID NO: 79 - DNA, 2000 bp, Artificial Sequence
  Synthetic
gaaccttgggtccttatcctgaaataaaaagaaagtgcacgtctccgtaatatatggatgtctcagtgatatccacgattacatcaagctgagttatttttaatgatagttgactgtattgcctaaaacgtatctgtagtaatgaatacataaaggtactggtgattgagaagttctcattaaacgttaaaatccgcatcatctgtaaaaggtgggtaattgcactatagagggtagaccacgcctgtagcccgcttagaacaattcttgtactatcatttttaagtccttcaatgtctatcataagtattggacattgcacgagaaaacacgggacaaaatgctcgtcgtttgagactatggatcgctattcgggtcgagcaatctgaaacagatattgtcatgtttggaaggtgagcccattagtagtaagcgctttataccactattcaggagtaataatttaaggagtgtaacagtatgatgtctaccggtacacgggagattgtaatacagtagtagctccttatggcttgggaataaattacaaactgaacgctttctttagagctctagtgtcctgatttatgggtaaggcgtattatctgcaagtctcagttcgggataggtattccgtcatctaatattacctctagggtgtatactaccatcctttgcagactataaatactatctatcgtcggcactgatagatggaggattccttgcaagacctgatatctccgtctccatgtctagtttatagatttgccttacaagttcatttatgcatgtgtaatagaatgatttatatgaaccgtcatagttccattttagcatccgagcgtgtgtcctctctcgtaattaggcgtacgtcgaatcattttgctttcactgtaaataggcaaagcaaaatgtagcaaaggaaggaatgaaatgatcattctcatgctacatgtgtccttatacataaaaatatatatacttgattaattgcacatgaatcacttacattcgattatcataatacatcccccactcggattgctccacgaccagatggttaaaaagttgaatctgtgctttgatttttaagtgagcactcacgtagtatgaaaccgctagctcaggttttttttggggatcgttcagtattcacgaaagaagaatgcggcggggtggttccacaccatatcaactagtgtttatagttgcttatataacggcaaccggctagtaaatggtaacttaacagtaaaatgtctaggattagtaaacatatattatggaggcgttaaggctgtacgccttgatagtacacacctttttacaatcacaatcctaggttgatctaaaaccgttgacgtcaagtccattataaaatcttaatcgcctgatttccctgtcctaaaatgaagagattaaagaagtgaaatatatccctaagccagaagtgggagaataccatttggatatatgcgagcttctgccaaatcttagagatttctggacttttcaattatccaatatgaggcttgaggattaccaactctggactacatgacagttccacagaaactatttagttagacgcagagccaattagaacctcgacaattaggtaaagtaaagtttacaatactgttaagtcgcgtaaaaaaggttgattcaactatgacgggtatagaggaggaaatagaggctctcgttagctgtgtcgttggacatagtaactttttacaaagaatgttagagctgttgaatatttacgcttatacaaagtatctgctgtatcacgacggattttatccatgcagggcagtaatccatcaggcttttggagaggacagccttgggaaggatatcgtcacgaggcgtttcgcactcagacacccgaaaaaattacgaggaaatgataatcgtaacgtggcgcctagcgctggataattaccataatttaacagaggccacaacaggttttcacccttcaatgagtgtaa

SEQ ID NO: 80 - DNA, 2000 bp, Artificial Sequence
  Synthetic
gattctgtacaattgtttcaaaatatagcttaacacatttgatggaataataagggttccaactagatatagttagttaggagttacgggagtggtgctcgggtacaccgaagcgtttatgtctaagctctcttctgagggggctcagacagctggtacaataattcatccgagccgcggtgaatgcggcatcaggccccttctatacttataaaagagcatatctaatttattggcatattcctgcaggctacataaagtcactcggtcgaggcatccctattcgggctaaatttcaacacgtctggtttgaatagcgactgttttttacagatggcttggataaccaatcaaccttcaagaagcacagttcttatgttaggaaccgtatgcaaccgtagactcctattttcacttgcgtgagcattcaacgaaattgggaagacagatggacttacattaacgtatcggactacgatcgtaatatccgtgatgtgagtattatagtatacaagagtgaggagatggaaatcatgacggttatcccacgtagcagcacacgcagatgcagaccagacagatacgaataaacttttttgtacggttgcccggtaaactagcctgggatcccgcgaacaaatgttagaataaaaacgcgagagacttgctttagtagcttttcatcaggattccttgcaaaaagttaacacaaagtaagcgtgttgttagtaatgtaatgtttgtgaggtaacactgtgggttaagtagtactaatgatctttctttgctgtttgactttcaaaatgcgtggagttcagtggtggcaaagattgtttaagtcttacgtattggtagtactcgttaagcttgaaagtttcgattatctctttttattccgatctgaaatgagcttgttctatccgaagctgaggtagtccacttagaccgatctatcgctaacgagaataatacttattatttaaatcctttctcatgccaatagaggagactgtcatggtaaccggtatgcttgtgttcatattaattctaagatttgctacaggattaagtctagttcaagtcctattccaaataccacaatctctaaggcctcacacgccttaacagaaaggggattatacgcgtcggttgttcgttatgccttatagtactcaacccataaatagatcgcacataagagtatgaatcggttgatgaaaaagtacataactcactacagtgccggatgagagattcccgtgaattaactagtggctacaaaacgtaacgtgcgaagagcaaaggtggccgcgatattacctttactttcggtgccttagtaaaagaggataatggcaaaatgaacgtcctgggcaatcagaccagagggaatatgcttagctattggctttgtaattgttgtagtttttaatggttctaaatatcaacaaataccatcatgatagttaccgatcagatgagcttgagccgttgaaaagaatgcaaatacaaaatcttgttcattaatccgatgcaacgtgccggcttgaaattcattttcgaagtagtgcgtccccgcgtatagacgctacagtagctccgaaggtctattgttagaacaacattttagaaacgggcctaataggagttcctcgggaaaaagaggaagggacaagttgattgtctattaagatagatgatcctattatagcgatgtcaatactacgcccagtgacaccatcaaaatagactggaaatgatggtacgattggatgagaagatcattagctgcctttaccttcgacgacttcgtcgtagtgagggttctgaccaatgtccatagcagttgaaagcgcgacattactcgaacaacgctgtggtcactctttaatgattcgtataatgaatcttcctctgcaacagttggacagaaaagtggcttcttgcttaggacctagctagactttgttgcctttctatgtaatacgtacgcaaattcc

SEQ ID NO: 81 - DNA, 2000 bp, Artificial Sequence
  Synthetic
cagtagatgaggataagcccaagtatcgattccaggaagccgccatatggagatatagaggtatctctggcttcgcgaactcacaaaggagtgtctcgatggacctccataggtaacaaagatcaaggccccttaccaactcatgttctataaactgacatctatgcaataaagttaacaccagaaggtgggtcagaccacaaaccacaaccccgctcaattttagaacaaagtctactaagaggtgcgaatcaagccgaaaacgggagtttattgtccatatgatgctggatcggattattgtattataatagcctaagatcgtgtctccgatccaaatgcgtgtacgcatcaatcctgagagatccgggatggttgctggggttaataacttctcctttatatccggatgactgctaattcctcaaatgcaatcattctggaattatgaggcctattaaacgaatttaacagtacctagtcggtagaaacaattctaccccgcatccttaagtctactttcagagctactggcgcctttgacgcataggtaaaaccggcgactagaggaatgtcgtatcaagataagccctaatttacttatgctagcctgtgttcgataaataagatgtctgaattgaattcgcgcagaaaccagtgctgccacggtgaagagtgatcggggcggctatcaactacgcggtgaactaccccaaaacatttaggacatgcgaatatatcaaagagaaatcaattccattagttcgaagatgagcacgatcgttactaactgcagacaaagaaggcactattgatagaaccgattgacaacccgaacgtgtaccggagtttggatcagatcttgagactgcgcttaaaagcaagaacccatcacaaaaaggcaatagcattaggaggaatcgcgcacaagtacaataactttttccgtattttaataatattaattgtccttctcaccacgaggccgtttccttcgtggaaccagtcgtcctactttctctccgtaatttcattttatttagaataaaggtatatacggacgactatcgttcggaacaactaataacagtgcttggaggtgaatagaagtaagttgaactgagctaaagtgaacaactacaattcgtagccctgatttcattgtcattttttttctgactcaacaccccaaagatcgcgcaaagaataaggccatagctcaaacccgaaaaaatcttctaaggcctgataacttagttattatatgaacaccggtaatccctgcatgcagcatatatgaaataaaatgccgtcgttttcattgtttcgtataagtagggaacgaggtccatgtgctattttgctcttttatgtgtgcccaaggggtactggaatgtcgagtaatactcagtccttcaatgctcatcttgtgaccaaattcattggggaactccattgggaaaggaatctgtgagagtgaatccagactaggatctacccacattgtagtctgaattttaccttctagaaagtaccgctcaagttgactatattttacacaatgtgggctgatggctggtctccggttgaggaaggatcaatcatactcatcatgcatacatgaagatatactagtatgattaacaataggttttcaaaacagacactcgacttattgagcaccctattggctaagcaactgcatctgcactagcaatggatcttaaggcatcatataaccggttaggtactttcttgttaggtagaacaacacggttgatcaggccaatcgctactgaagtaatgaaatcaataaacactgagtcttatgaagtactattacaatctcctagggtcgtatcagacctttgttatgttttaaggacaatgcgggatctctcatccaaaaagcgaaattgataccaggcattggtagtcaagattaccgaattattttacgtaggtcattatatgcctgcaattttggcgctttacgctca

SEQ ID NO: 82 - DNA, 2000 bp, Artificial Sequence
  Synthetic
gtttaatctccttgactaacaggagtctcttgccaacggatgtacgtaaccgtatgttaagacattatgaagagttaatattacatgcaaccattcgatttgccataaatgtaccgaacgccgttatatttacttactggatgaaagattcaagaatcaatataagttaaaatcttaaaaagatcaatcatacgtataaagtctatttgctattagagacgactgtctgatttgatgatgcagcgcgttgttataaacctcataaataagaggcggtggctttcttactattagcacaagtctcactgagtagtagaataactcttactctatatgtttcatcaggtacgaccccacgtggcaaaattacattttgcacacgaggcacattaagaccgaagagaacatttggccgagaggtatgtcaaagccggcttaatgatatcgacacaactcataaatggtgaaagttataaccaggtaatcttatgggattctgtggagtaaagcccattggacttcggaataaataagcaagctaatcagttataatagcatatatgttaataccaagcgtggaatgagcacattttggcagtttaacactaagcttgataaaactcgtagagtagcgattggacactacaagacgcgtgtttcgctagagacgaaccaccttgtgccaacagattactctgaagctcgcctatttgtggaagtaaatattacgtaacggttatagcattgttaacgatgattttgtcgagtaacggtatgaatttatgaaaaacgtcaaacaagcgtgatcagtttcgcatgatcgaattgagtttttgcccgcgcagggttcgcgtcaaaacaccttagagtaaatacttaagaggaatcgctacgtctatttgtaaaagtccgagtacccaccttggaatccccatttttttttttccagtcagctcaacggttgaatccacgtgtccgaagaagctctgagcaaactatggtgtcgccgttctaagcccatttcaaacgttatggagcgttgtgcctctttgttggcacttgttattcaccgcggcgaagtaacgcgctcgtcaagcgaatcattttatgcctactcgggctatagttaacggagttaaaatgcttcaagtgtaggtcgacaaaagatcaggaattcgagataaactctccatgtgaaatagcaagtttacgtcctcgtttttgattatagactaagattacgaattctttagcgctggctcatttgaatccaaaaccgtagaataagaaccccagacttatgtcctcgaaattatcaggtaagagaacaaataattcacgagtactgacagtataagcgcttatgtgagacgaccacgtaactacaatttataaacttgaccgttattatgtagtatttagtggctcataaaaccagcttagcttagatctgtgagactgaccagctgacccacaagacttttacattgaagttgcagctatatggaaacgtactttataatttcttaatgtaagaataaatttgctgtatcgctttgttcgtttgaactcttttctatgtaaaaggctgactaacccaggaagaggggagcatattttacaaattagtaagcgctctctcattcatttaatgatcaccttataccgacttcagcctatggaagatcttgcgctgttgcgtacctacagcgggtaaacggatgtgttaaacacgatagtaatagtaagtttccgttaggctgtagtttataacagtaacataagtgctaacgagatcaacacaattcaagttgcgaaagcaagaaaatcttgctacatatatcttagataagtatgaaaacatagattgcgtttttacaaaaagtacgaaaacattatattctcaagctcacgctccatgaacatgccatggatgcgagagctacttaatattatccggtaattattaaagtaactaccggttgcgcacaacggcttaa

SEQ ID NO: 83 - DNA, 2000 bp, Artificial Sequence
  Synthetic
gactcttcttctcagtccacgtttgaaaatcagacaactacatattcaatggaagcgctgagtcggagtggctttccgattgactgcaggtgtctggcgatagattattaaaataaccgaggacctcatctgtgattacttatgttaacacgtcgttacaagcaaaatgtacagatcgtgtgtgggttaggggttcactagaatcggtggggcaaatttgccgcaaccgatatcgtatctgtcgccatttagtgggagctgggcgtgctatcagaatttatttaaacggtttggggacaaaagaggaccttatactggtagtataccttctttagtctttgctccgattgaatacaccggaacctaatttgtaaagaggcccagatgttggacagagtggttatgagtgcaggtttatagttcaagcatcagaatagtattaagataaaactgagggctttcaggccttgatttaaatgtgagagtattgtcaggccatttggaaatatcataaaatcctttgtgccagatagttatgaagctgcttagatccacttgccttcatttgagtctgctgactgccaattagagtcctcctcggtacgtatgaatagaaaacttcaaatacgattctccccaatttgctctgtgcagccttgccgatagtcctttatgtcatacactaggtgtgagctccaagggtcttggttccagccccgcaattcagataaacataagccccagtagcggaggagattttgaataccaaactaactttataacccgcgcatggccagtgccatagcgaatgcgcggggagaagtcattttagaagcctatcaggcgatcccggatcattaccctcgtataataaatagccttagctgcaagttcgtgtcgccgccaacgtattcggtatcagactctgatgtcctttaatagtgattatgacgactgtcataaactttgtagtagtgtatattatcgattgcgttttattcatcttgatgatgggatacatctgcacttttgagctaatctaagatcaaatatctattttcacgatcccgctactacggctcgagaaagttactttaccggaccgggcttaacacaagacttacgacgtcctggatagaattttaggggtttctaaattgatccggtttgagaacttcttacttatattccagtttcgaggactaggcatttcttcattaagaccgaggcatgggttatttttatattgtgatgcaaatcggtttgccccgccggagagactacatgccagttggtaacgtgacaaggcatgtgcaacgttctttagtgtcgctacgggattctgaagtctactgcttacctgattataccacggttcaacttcggttacaaaggatattcgctattgcacgggatggaaatttattcatgtcccaaaaaacaaactcgacaaaggtgcccacatgcggcctcattttacagtgcacttatgagctattgcgagctccctccaaatattggtgggacagttaataaaaacgatctgataaaaatagtaggtatcgagacctaagattggaatgatcacattcgcgtgttataagattggagatgttctaacttggatgaaaatgttagttacaataaccatatcctggttcgaagagtattgagatggactttcgacattataatatgatttcagaaaggtcgcacatgactgatcctttcctctgcaggtggtcctgtcatcgggtatgtttttttcctctagataaatggatattgtaagcaaatagtaattcctgcatgctggataccatacatgatgtgaccgccataagctaaccagcttctaaaaaaatacactccttgctagtatggtgattagttacggtgcatgaaaatagtaggaacgctgattctcgttcattttgtgtgcgttccacgacgaatttctgttcaaagtcctgcagatcttattgagacctttacagcac

SEQ ID NO: 84 - DNA, 2000 bp, Artificial Sequence
  Synthetic
tcgtaggctaatagaaacagaattatcaattccttatttaatacatcactggactgagtcattctctcagagcaaaaggtaatcgcttcattaaggtattgtctatcctgtaagaacacccacgccgtggatatatctcaacatgtaattagggggtacatgcagtgtcgcaaaattcaagcgcgaactggggcatttctagttatgctagctaatctactcttgtaaaggagctttcgactaaaaactgccactataatctgattcaatggtggtaataagcggtaatctttaaccgtgtttttgctgtccgacttagtgaattgatacgtttatagggaaaaaataggtcgctcaatataccttaaagataatatcaccggcatgcgcctatgaggtatcgatcctgtgtctatgaggtaaaaaacgagactaaagtttgactgtattaataattatgaaagggaaccttgtagtcaaaagattaagagcaaacccgtctttcaatgacaagacatacattggatgcctcgaaattgattattaagtaaccagaaccaatgattatactaagagcttattcctttctccgcagactcttaagaaacaaggacaactgcccctgagcaaccagcctgctgatacgtccaaacaacccgttatcattagcctgtattgagctaaaagcacgtttattacttacatggcaagtattatttattatgtggctcgtataggtcgggtatagaaatgttgcacattacaagaaagttcaatcataaagcgaatcgtttatgttagcagactttatctacagttaacacgaggctagcgagatgtgctacttttcaagtgtttggaatgcatccgaggtcactataggcaattctttaccgcgatcaattcgtatttgaaacgcccggctagcctcccatagattcccagtcaaaggaatcaaggctgcgccattctgtgatttactccctctttggacaaccaacgtactagcctgcaggatacgatgccaacattaatttttataaccgtgagatcaacgcggtcaaggaaaaagttaggcataatatcgcggacaccctggcgtgaacgattaacatctgcgggatatgaacatttctcgatttactttaatgatacttggcttcataataaacataatacatccccctgaggttgataaacgttagaaacttaggcgagtccataagcgctttaaaggatcttttatcacacacgcgaaacattaccattcgataaaactcttatcactcatcccgaaatgccagtttcgcacatgcaaaaataagccttcgagattggtcacgcccgatcagtcgtctttcgctacctaacctatgataaaatagttcttaggagtcaggcaattgacttgcctgtgtctctttggaggcttccaagttcggatttaagggtatatgcctgttgtagtcggacaaatagataggataagcgctttccaggcggactacactattagtaactatcagcgaatataaatgtactcggcagcttaagcgtagacttagtactcgcaggacctcttgctcgttctagcatatatcctggtcgtttttaacattttaagctcgaaaaagttgtcggaagatgactccattagatggacgattaacgaacaaaggtctgtgaatgacatacacatctgatcagtattggccgcattcgcaggatagtacatcgcggggcagacgtattaaatcaacctctccacacccgggtttcgttttgccattgttgccctcgacagcagcgtttcattaataggaggctttataatacgtccagaaggtgtcagaggcctacgagctcacgaacgtatcctcataaacttattgtgtcaccagtcaagtcgtattttatctcctaaaacgacttacccacaccttatggaggcttagcgatcgtgtatatatgcttcttattatagtgcaccctgggttcta

SEQ ID NO: 85 - DNA, 2000 bp, Artificial Sequence
  Synthetic
attgggcatttcgtcggacactaaatgaacattaaaggattgatcttagagtgctatattgaatcactcagcccagtccttcggacttccttgtatttcactgggcgtatactacattctcaaaataattttgcgagtcaattaaactagataccacctatggggggtttcgtcttggtttcaaattagatggtagtaagtttacgtgaacaccgttgagacgtagacggcttttatgggttgtctgtgttagactcattgagctgctcatccgaattattcattcagtactatttagcacttggacatccctgctagagctctgcgaaatgcggtattaggtctggggtgacctccagctcaattaatttacaccggtagtaaccaaaggttagttaaactcacgaaaatgatactcactgttttgtgtatccttagttatatgtcggcggattcaaccttcggataataagtaaatggtctcagatcgtagctgcaaaaaatcgtaaagcaactgttgttaagattggctactcctaacaaattccgcctccctcaagcaggacacttcggaatacaatccggaaatatggcgtgaaccctctatgatcgactgattccaatcacggttcagtccactctatctaattaacttatcgggtagatactagaaactcactcaaaccgtattcgtgaaataattattcggagtcagtaagcaaagcccagtgtgtattttacacttaattggctctctgtcaacttcttgcaaattaatccattacttgataataatatatcgcgttcaatggcaagaaatccaccgcagaatcgcaaatggactccctctcatctaggttaaagcaaaaatgttgagattccacctaaaagtggatatagaagacaaaattatttgtaccaacagtaaacagggacggaaggtgcctctcaggtagttactgaatacctgttagacgggttctgcccggcttctatgacttgagattatgtggttctacagtatatcatccgtctaggagtgaacctaatgaaaaatactctaggttggtacgtattcattcacataaacggatgcgatgagttggcgggttggaagttctgttaatgtcgtaagtacttataggctgacaagaggtaactgtcatacgaaaggattcggtctcgacggccgaactctaaaaggtctccttttccggagaacacaagactcttctgcttctgaccgtatttggatagatccatcggcggtacctttgtttgttggatcgtaacatctcttttgatcctactatgtgccaactcagttagttcgcgctgaattaagattcaagatcctgttcatatcttttataaaacatgtggatgtcttaaaactcatctcttcaaacgccattgctcgtttctggagtgttacgggttcggagtagagtggtattggatgtcaatatgtgaatttatccactctgacatacacaacgagtccgagaattttagatcgtgcctccaaacagcgctcaaatcttacaaatattaatgtagagccatggccccatgcagagatgttacattcgcatggatcaatctaagtttgtacaaaagaaaggcacttcttaatctgaacttcatatcgtgtttccctagcgattactatgattctagtgtagcgttagttgcttatgctctttatacactcgaggtatcatgtaccaacaacctagcgaaactgatactgagaggttgcagatagtcttcgacgatttagctactgtcatttaacattcctgcctaaaatagcttccgtccactcacgtactggatctcattctccgcgagccttatagagactggattacgtatattcaataataatctactctagaccaccgacctcatcccttgtttattgatagtggtgtccctagctgaccagtcttgttgggaagaagcatgtaacattcctattagcgccaacaacgcgt

SEQ ID NO: 86 - DNA, 2000 bp, Artificial Sequence
  Synthetic
agagaacgtgtcacgtactaagtgcaaaagaggctgggttttttttgttagcttaaaacaccaatagacacaaatccatggagatttaaatgcaattattaatcttgatcgaattgtcttttagccgacaacctgttggtcccgacaataaatttaacgattgtttttatcctaagatcaaccgttgacgaacaaattaggcgaaagttatattagtagccagacgcgtttggaaacaggcaaaaactgctagaatacccgtagaaacctactggaataaatgaaccgatacgttaccgtctcaggaactacttaggtttgatagacagtggaatgccatatgtcttttagcgtaacaaccctaaaaccttattattggaaatttaccaggtaggatgtcatgtaacacgccaatccaattcatgtcacaaagtgattaggtatactagcatttataacttgggtaagtgcatctcatgtaagtaccgatgggcgtacctcttcgatgtattaaccagcacccacttcatacaagttcatcggtaagtggtttacaagaaacatcataaatagaaataacacctcttcagtgataagcggaaccccgtgccacttgaaacaatctctcgcagatgacccttggaacagggctgacagtttgaagtgacagggtgaagtcattcctttacaatttaagccgggaaatttatcaacactaaacgtaaaataaaattggcgtactgcctggacattggtcgcaatgtaatcttctttgttctcgtaaaccaaacaataatattttgaatcgtattatattgcacaggtaagccactgcaattaaattagagcccatcacttcccgggctaattgagactaagtcaaattatcctttcagacttctttaacctaaacatgaagagggttttggaattgttaaagacattccatggggtactgacgtagtaccagccagagttcgattcttacaattcacacgtataggtagagggtcccacagctacatatcctatcctgagccgaattctcgccattgttagctttaaatatttcgagccagacctgtggaatttagtgagttgaagactatgggagccataccgaagttgctaataaaattgtttctaattactcttcgtacatcagaggcacgccatgtgtgtgattaattcatcttgtttcccgtacaagcaatagcaatattgctcgcatcacgtccaccaagtaattattgtatagttactttgaactatatctctgtagcatttcgagtggtgctcagaggcgcggatcttgcctgtcggggattgtgaaagttggtcagaaagttacaacggtatggtattttagaaatcgcgaacctgattgcgtcctaacgcgatgttattagtattcaacggttggtcagagttatatacccctagagaggcctatggagatagacagtctcgcgtatctcatcataactcttgatcaatctagtcaagtagttcacgggactagccgtacacaataaggaacctaagtgcaaaaccactctttagataaggatcctgcgccatgctttgagccgcagcattctctcgatgagtccagcgtggtttgcaacacttagtacataagatagttaaatacagagcggtcctattttgaaaaagaaatcctatggaccgcaccagccggaggttacctaagacttcggacgaacatccttgtttaaatgtatgactggatgactgattttcaacagagcgaggtccaagaaaaactacaagccacttattaaagacatgagtaaggacgagttattgaaactaagacatacgtgggatagctaggtggcataatacaagcagataaccccgtacgattcaaacgatcttaacaagtattttattacaaacgggcctggttttaagagaaaaacgtgcagtaccctcaatatgagtaataagggaagtgacagggagcactcggcgat

SEQ ID NO: 87 - DNA, 2000 bp, Artificial Sequence
  Synthetic
agggcttgcatatccacaaaaatgaatttatctaggttcaattacgtgttatccactccagcgaaaacttgacactaggattattgtcttttgtcgacacgttaatacagcaacgtccaagagatctcttgctttggcttgaacttgcaatattcacgggttgtttccattcttacctcgactggctagctgaatgacctttcacctgggttacgatgtacgcggggcactgtggcattaaacgaagtcattatctgcaccaacccttgataacaaaataaatatggtctgcgacaccttgtgctgggagacaaaaatcttctgtaattggttctgtacgacaggattagttcctctttatttcttaccatgtttcctcttccagcattaagatggtaaattgaatgtatagtgcgcgatacggagcacgtgtcagttgtcgctcggtcgtcgcgattattgcttggaggatcctaataaagctaaatgagtggagtagtagtatgcgtgtgtgccggccgtaatatctcattcacgtgcatcatagcgcatatattcgacacttgtaatcccgtctttcgaagaatctaggttaaatggatactactttttacacacgcatcctgcctctcggcgggaaatatgttattagaaacttctgaagttgtctggattaaagtactcatcatggctaaaacactctatttttggtgtgaatatagctctatttacttctatcgaggcctcgttctagaggttattagtgacagtccgtccgtaaattttcctgtatactcgtcttccttattagggttgaggtgtactgcatgtcttatgctatacaatcagcgtacgatcaagactgtaatatgtgtatacgaccacattatgaatgagggtaaggtgcgatagtcagtagctgcttgctattatccttaaatcgaataatgcagcgcttcaacaatagatcatatgtatttcaagcaacaattaggggattcaactagagatgctaatgtaggtttgtgaatattttggtcgtacattggtagggcatctgattgcatgtatacagtcataattcagagcgacgctctttttaaccttgggaaaggccgtgaacgaatgcgattaggccaatctagcgcatatagttaattattttactctttatctcttgagcaacagcggcaaggaaacctgggagttgctagacaccgagtagaaatcccttacttcgccagcggatcgatctgtactacatgcatcttctactaatggttgaaagtgaagctagtacttatttgcatggtgcacccattcttacaaccaggttgttctaatgtcttttcatcaattcttagcggagtgggcataatgaaagtataagaatggaagtgttctattttgcaaccggagaccacatgaaaggatcgacacagagatgcaaacagtgcatacattcgatgtggcatagaccaactcttgtacgatttaatgtgatctctgtcacaattcgtttaggtgtctatggtaaaacctcagccacaacatgtatagtcttacaggcatggctatcgtgatttaaccgtgaataacttgtcggtaacagaaactctggcacaggtgagcgtaatcaaatcaacttcagtaatgaggacttctaagatagttccgaatctgttcacagtattagcacggtgattgagttctcttctaatattcctatctttacattgcgtactgtcacagaatgctgttgcctctatgattttacaacggcaatctaaatcgtcgtatcatatgttcagaatattaaatagctcaactccgtgttgagtcctaagataaagatagaaacattgactataaaatctatccattgtaaaccagactaatcatgcaagcacaaattagagggcagaccgcggccattggaatcatttatatctttatcgtttaattcacaagaatggctaaatgccggattttgaccgggc

SEQ ID NO: 88 - DNA, 2000 bp, Artificial Sequence
  Synthetic
ttacataacgactccgtcgaagccgtcccggacatcgagtctgacacttacaaccctgagagccgcttccctatatgtctatagattgcgagtgtatgccactgtcattgcagatttagggtcaccccaaaaacacgagtattattagagactacgaatcatttagcaaacaatttcgcgaagccctaattgaaaaggcaaccgattcacccctggatagataagctaaaatagtgttatgcggagcaatgttctcatttggacccatacactctattccttctgaatgaccttcgaaatacgaataagaacatggcgttcccaatcatccatatacccgttcaggctgagtagccaacatttcgtattcaaagatacagttgacaagctgacattcattgatgacttaggggctaacatatcaggccttttcttaatgtttaaatacttgcctattatgtggccatgaggagtgcgatgataccaatgttattggagtatcgttaaaaaaattcggtagtgttataattacgaactatagcttacgggtcatctattttaacatagtgagggcttcttcacacttccagtcgtcggtctgcatgaaacaaaaatgagttacatttagaggaatgcggggtaggcacaactaaacacaaggattaaattcgtcgcgacaggagtacactaaacgtaattaaaaagctaccaggcgaaacttctatttacgggcaattacgaatcctatgacacttcaaggacctctcattctaaaatagagacagcctccactcgagctccgattgagctctgctctcttccaaacaagaacctccgtgcgagcagcatatagcgagcattcttcggaaggacctatatagatcggtcagttgggaaatcttacaaaacgtcgagcatatattatttgccgtccgcaacctatgcacaggggcctttaaatcagtttatttaaaaaatctaatttcaaacagtcttgcaataggttaggtgggtatagagtatcaaaaatacgtgactaaaaacaacagaagttgataaacaacagtgattttcgggatttatgctacaccttagcgagaaacttctgttaacattgtctatgctttgaaactatgtaaaggaattcgtgatatggtatacctaataggcccataccattaaactgaatcatagtggacgagaagctttatcgccctctaatgcgtagtgacgaatgaaaatcagacaaccattatagaagtccgagtcagccacggatgttcggaattgctatatatacgcatgacttgccaaagttgtggtttactgtatatttcgtattccacaattacatatagctaaatctacgatcgcggcgcggtataagatttcaaactcggtaaacttgaatgatttaaatcatccaattgttttatggatcgtggcctggagtttggcaattaattaaaggatatttagctgaatgtgtaaaataatttttaacccaaatgtgtctataatatgtgctcggataaagctcaggcataaccacagatctacgcgaccttgtgatcgtccttgtatgtgtatatagagcaactaccaacagttgttcagacgcaatcaaacgatagctttacgataggatgttcatttattaccaagtactattattcactctatagggttattatatcctctactactccggggtgcgcaactttccttacgccattattaacggaatgagcggtaagcggcaccttctatatcatcgtcataagagtgagatgtaatgttactatgccttatgcttgccatggtaagccgaaaataagaagatcacaaaatagcaccatcttttccatagattctcataaacattgatgtttgagcaaaataacagctattacaatgatgtaaattattataaatgtctaatcataagccagtaatttcgttaagcaatctagagaagtatcttaagagcgttaagaacc

SEQ ID NO: 89 - DNA, 2000 bp, Artificial Sequence
  Synthetic
cctcactgagaccaattatgacttttctcttgcaattacacaatagtgcgttaagtactgaaaaccatcctcaaggctaaatgttataagatttttcatacgagtggcgaaaaccaagtcaaactggttaaatgatgtctactacaagtttgggcttggctgacaaatttttctatgagctactgtaataatgcgtcttcatacgaacgcactctgcccataaataggcgatggacctaatacgtcaagcccatcttcaaatagtttttcttgtaaatttttgtcttgacagacatgatacgttaacgttgtctttgaccattatatcttcgcgatagggtcgagttcgtatttattaaattgatgaaattgcgacacatatcacgtgacttaatcccgaaaaattagagttcttgcgcttgtcataggcatgaaaagctcccctcataatacgtttgacctttaacgtatgtctttaacatatgttcctggtaaccaggatttaaagtcatggtcagccttcgaaaaatgtgagaagatcgcgaatacatcacgaactctctcaggcaaacatctcatccaccatttatatagtagatgcgctacccactgttaacctgtttgagatgtcgatttaaacgttagaaggtggttccatcgctggattgcaacctttacttaaggtcgatgatacgtacaatcgctttactttaagctaagttattggcatactactgaaattcacttcctggcagacttgcgttgctctcgcaatcccgcagtcctttatgatgtctaggcgttttacaaatcgacagtcattgtattaaagtcattggattgtacggtgtaagtcgacagggaacgtgttgagttaatagtaaaaggttcagattcttgcaagcgcgcttttctatcgcctggtttatcaaactcatggtgattatatattttgcaattcatcagccctcatatgttggtaagactcggattgggtcgacgccagactaacgtcataaatgttagaattattaaagacgcaattgtttatgatactcactaatgggtcgttagatacttattgttttaaggcaccagcctccatttgtccgagtccaggcccgagcttgggcgcaaaacttttagtatctaactgtgagtgacaacctttagagttctctcgtatagaaggtccgacgtcagagtatcataacctactggaattggccgggttcgcgtgcactctcacttcctgccagaacgcaattaagcatgctggtagtctcgacccggtacctcactctatcaaatgaaactatagtatacctatcgatcttaagatgtgggttctagctgtgactgcccgaagaaatagtatttcaacgacccgatcgtctaggagcgttgtgggagggttcaatgctctcgtatcgattcccaagacgttgtggacatactagctggcgaataatactatgtgtagtgaagtttgcggtaatctgcgtagtggctaattaagaaacaccgagccgtgtcttttgcaaactcatcgaggcgttgactaaaatgtctaacggttagggcgatattttatttttacccgcggtttattatctatgagtactccccattcccatatagcgtgcatagtttacttttccatatgttattagcaggctgtccgcccaaacgttgcgctagccaccgttagatcacagtcatattatcataacgattaccaggttatagtttcactgactaaggagcccataaatgttcattttcactagacatgctatgggtttggcccgaccaagattgataaactgcggtaatggcgatatgattaaacgattaaacttttaactaccatggggagacaagacttcttaactagtcggtatggattgctgcttgtaaagctaaacaagctgaatgtaagaacaggctggccggttcataacactatcacgagtggctgacagagttttacttatagt

SEQ ID NO: 90 - DNA, 2000 bp, Artificial Sequence
  Synthetic
attcgcattgtttgagtagccgagcactagtgggatcatttaccttctcgcggaagagttacaaaagtactgaggaaatatgtgaattgttatagcttttaggaaagtaaacatgaaacaaggtagaacagatgacgacgtgatacaattatttacacaactggaaaattccgtcaaagttttaaagtatattccttgagtcctattattgaatattcgaaaggtagtcacctgagttgtcccgtaataattacataagtatccgtatggcaacaaatatctcctagatccgggccgcggatagttttcgctaaagtatctaaatcgaacttcttagcatacgattactagactatcaccttgagtagtctatatctctgcgagtgtaaaatgcacacgccgttaaatcgcctaaatgcctttccgtggccattatatgccccacttgctttcaattcattccataaactatgatcatggacccggttgcgagatgttacagataaagtcgaaactttcaagagcagctgacgacaggtaaaattacgatgcactgcggtgtaaggaaataatctccaggttgcaatagacatttaaattgtagaggaatagagttacgcaaaccaagcccaaggatctaccgaacccctctaccttatacaaactcgtcagccgaaatataccaaatagcacgttgcctagaggtttacattaatcattttacacgatccctttactattaatatatcgattccgatctaaaaggcgtttcaaggatagcaatagtcctatcaaaatcattcagttactggcaatccaaccaattcgctgtacacgacggggtgaggtcgtaaaatattatatgtcatagatgcactgtttgcgaccatgtctagcatttttcaatagctccacccacgcgttggcgacccattgttattcaaaaatgggccgcatgaagagttaattcgtcttgttctgacataagtgttgaccatcagacaatagacgtataccgctggttacctctaatcgaagatccagagctccttatgcaacgtatagtaaacctggctcggaaaggggttactcttatttttagcacctacattcgggatcaaatcatatgcactttcaagatggtgctcactataacacaataacttgggtttccagttaggatgaggaatccgccaggttactctatgaagtcaagctcttccgtagtttaggcgacgcttgacccgcgttcctcacaagtaacgcgacagattggagcaatagcgactgcttcaccatatagggacttacatacagatcgaatgatttgcagctttaacaacccataacgatctgcactagatgcgatgagatctctgtaaaacgaaacttggaattacccagagcagttctaattaagctttttcgataatattacacagcaactaaatgagcacgtatgctcaagtgtcgcaaaatccttattgtataggaataggtcgttgtcacaacataggtctgtcaccaaactcagacattatagtactttacggagcatgtttagacataatctgcacaatgctgattagtctcagtgtggtcaaattctttaacgtctctgttccaatcaaagtgagcagactgattgcatcacaactccatcacttaaccaattattaatagtccacacaattcattcactcttcactgttcagcactcagtcatgctctggatattccatatttccccgccacatatactgagtttggtcactcatatgttcgctaaaatcgatttttaagccattcttgcctattaacgacggtcctaatcgtttcccttcaccatggatatacggtacgggccctattatctgcgttacgcaatgtcaataaaagatattctaagaagaaaaaaagataagttgcgtaagcgtgctgcaagagacactctctcttcgcagtaaactaatttttcctttaagaatacaaagcgaaca

SEQ ID NO: 91 - DNA, 2000 bp, Artificial Sequence
  Synthetic
ggattagattgtgccataacgcaacaggtaaaattattagaccagcaaaagaatcctaacgtatacaattttatcgtacataacccgtgaatcttattaaacccagccaggccgccttactttgctccaagtaggagcataatgcatagaagtttcagtatcctgtctaaagctattaagtcgaaatgagacaaaagtgacgagttattaacgatcagaaactagtctaaagggaaccctcctgcggccatttcttgaggacttacgtgcaccatatcatgaggtcctactgtgggaaaggaaatcctcagtttacatgatttgaaatactgtagtgacctgtcaatttactgatttctatgcataaaatgacaatctcaccgagtacgcataaatcagcgcagatctcatatattcataataatctccgggacgttattaaattaatttttttctagacagatattcagaagtccgacgttatacaagtgcccagtaacatgttctgagcaaatagattgtcgacagccccaattaaccacctactagtctttaggcactgtgtgaatgaagctattaagtactagacataatgtcattgctggctctagctgaagagtatacctagctttttttccagatttttgagtacgggatctgttcttgttgaacaaataatctggatggcgccatacaggcgtcgcctggagcgtcaagctcacataccctatcgtcaaagtatgttccgtcaaaggtgtctcagcacttaaatacttaaacaatccgagtttcgagttctaaatggttgcacaatatgcctggtagattgatataatcttgaagcaacgatggatgaacaaaaattattgatacttacttttacccacacaaaccgtctgagtgtctttttaagagggttacgaatatataaaagcggatcacgatattccaccgggaatagcgcaattagtcatatggaacatggtgtgaaaccacaactatgaaatctatccgtacaccaaccaagagacctaaaagttttacataatccgtttgctttcgtattgccctctatctaatgaaaacccattgacaattataaagaacaaaggttatcacacgctgcgtatttagagaagagaggacatgtgggatcaatgtggtcgcaaaaattatcactttaatcaacaccgattctaagaagaaataaacgtcgtattcaagggtactgtataggtacgttaagcgttgtcgtacactcagcgatttaactaacagccgggagaatgcataattatgataaagtgaatccacttagcgtctcgaatagaggctatttcgcttgcaatcaaatgcttaagagtatcctaaccaattttagacaaatatcagtatgtttatcgattaagctggacaattcctctacacagatgtttaagcgaactagcattttcatcctcccgactcataggagtccttcgttgcacagtagatagtcagcgtgtgttctcttctccaattgatatgctgaaaaactataggttacccgtttcggtcggataaagaatttgacttaattttcttgccgatagtaggtatactgtaaggcagccaatataaccgttagagcttgattagtatgatattcgctccttttaatgtatctacatctagctctggaaaacccggtgtagaagtaatgtattaagtctgcgaagcgggaatctgcttgtgacaaagattctgtcgcccgcaaacgtcaagtaataaatcgcagatacggtcagaaattccttctgcatttcaagattagtaatctattcgattccaaacatcctgctcctaacagaatgcgcacgggacctaatgaacttttcatatacgtttcatcaagcagtagtgttcggaaacgagacataacagggtacatgtgcatcaacctttaaaaaccaatctctatttggtatagtcgtattcgaaatccagtagtgaggtgaaaa

SEQ ID NO: 92 - DNA, 2000 bp, Artificial Sequence
  Synthetic
aattggagccaaccataaattggatggtagttccaaaattttataacctattctagtgtctgcaagtatttaggagataggtgaattacacgtcgtacacataaatatgataatgcgatcaagagtgaatggggtctatagtaatatgatgtaaaacttaaggatattgtggactgatttaacgttacgtagtcctgacaagagtttagatgccaggtcgtagaagttgtgtatccccctattctcccaatggtagataccgtgataaaagataaattcctgttaaggaagtcgaggatgttctgtggagtgcagagttctacatgtgatgagataacctaagagaaaaagtaatttatagattgcccccgttaggagctacacccgactatttgtttcgttaagatatttgttcgtaccatgctgttataacgacactccctcgaatcttattttatggcaattaaagatgttacaggtggcgttggcaattctggtaaactccgcactttacaaattgttgtttgcaactctctcatattgtatgcaatcgaccccaaaccctcatcctcgaccctatgaatgaaggttttctgtgccaaaagccattttactcaaaaattagcttttaatttggggagcttaatagcgaattccagaatcgtttcatggggattaggagatatattataggagtccaccaatagtctattgacttagtggttttggctcatgcacggtggacaaaacttcaggcgtgttatctaattacaacccgtattcatacatatcaggggtgttgatttcagagaatagattaggaaactacgagcaataccaattttgaagatatggtctactagtagctcacttactcaacattgctactttattcgaaggcccatattgaggaatactgtcttgttgagtaaaacgatacccgtaactttaaactataaaggcataccagaaaaagtgtcaccgcaggaaaatataagaacgtccatcaatatatgatgcaaactagagaaagagcttgataaattatcaaactagcacttctgggaatactccgtggttgcaaggttacagggttcagtcaaagagttattaaatcgattgatatacttattcaagtgattgattctatatagctacgcatatctgctgactttttcgaaacgttgcctggttgtccagagcatgttttggacgagaaatttcgcgcagatatcatgattacgattggcaactaaggatgactagcgtaatgagaacctggctaattttgtgtttcttattcaaattgtataactaggtaaggaacgactcgttcagaatgagttctaatcataatcttctaaaatactgacagaaataataatatatattatgactattcagaaaacctataaaaagcactccgtagaagctcttcaatcttagaatcctcacctaggaacctgaagattattgtattgacttattttgtagttattaaagaaatccaacgacggggacgactgcttgtatgtaatatttccgttccacaagccgggagtaataataagcaaccgtagaggagcaatgggtttttatctcacgcacaggatgtcggagtagcgagccgtctgagtatgttatcaccaaagatatatgtaatatggttaatcagctgatttaaagagaacttcatcccaacctcgaccgacgatccgattactgtttatcgtcataccttacgagatgtcaggtcctcgcacaaaccgccacaaattccttgtcactgcaagaataagtttgtccgcaaactgtctacgcgctaggtcgttgtatgtattgatgagccctatccttatgacactcggactgctagccttctgagatttacgacaggcagtctagtattaaacccttactactttttgctgtatattgcattgcaagttccaacaagttaatgaaacacaaaccgtgatcgcctcaccccacaaaaggct

SEQ ID NO: 93 - DNA, 2000 bp, Artificial Sequence
  Synthetic
gtaagggtcgaacctctgatcatattcgattactaataactccagatatatagaattgagaaaggcaaatgtattttaaacagcaagaaactgtttcaattcggcttatctgatgtacatttaataaatagaatgaagatcgagtattagaactgatatgaaagttcgtaacatcaggacgattagagtttatgcatgctaacaggaactgacctgctgacattatatcatacaatttcctgcgtcccgcttatggatggcgtcaataggctagtaacctaattgcagcttagaataaggagaaccaagtaacgacaacaaaatgaaaagcaatagatggcggactgcgctttaattgcattgaaatactctgggcttcaagtgttagttcattaaagctgtctcgcgatacacaaacgctgcgaagtggttccggagtaaatgtgaccaatgttagacagtgggcccgccatgaatgtgaagttagttactaggaagagtattctcagtttggtgtttactagaggtgtgcttggcgtttatctgggataataattgtaactcaattctattctttttcgttttttctgctcatatcgaagttttgctcgcctcaatcaacgttgtttgtatagcacttaggatcactctgcgcatagggaatgcttaaatcagggagttcatcggtgtccatcctgcagggacatgaaagctgtcatacacggactcgtaccggtctgacaatccgctttgcctcatagcaactattgagccgcattcgcgtggagctgaactatcagaatggctagaaaggataaacctgtggtgggtccacgagattggtcttcttatgttaatattagctcacaaagtccagagttagtatccatctcttccagtcacatggaattttactaattattgtggtatcattattataaaaatgacattatctagcatgactccctaccactagtgcagagctactatgtacataactcgctgtttatgcgatactccaacaagtagatacggtaatttcgatataggatgaaaaaaccttcataacagcttaagtttaacttcgagggtccgtgtaatcggacaacgcacatacgaagtggcacgacctttcatttgggctcccttttgcaggctagtaaacctagtatacatgaaagccgtcttgcttgtgcctacggcttatttcgttgaacgtacgtctaatagtgccaaggaacgaacacacggctagatcataatattactccaggtgatggtttcggtatttgcaaagtaaagataagttatctgattcacaacaatcgagaatttgtcctgtttgaacgccgaaatattatcttactattgctttactcagatacctccaataaattataaaatggcttgtttgaatgtgtatcgaaaccgaaagctatatcttttgaccgaattaaccaaatgctacgcgtttgctgtttattatgtccatcatcgctttaggttaagcttaataggttagggaaaactaccagcattcacataatatcctatctaggaagttaaattcacccatgtatactatactacttagtctacaatatttctgctttattctttatttccattatcaaagtatttcggctcttaaatggggcaattacgaaagatatgattctagctcatgctcaattgagatgaatttatgactttaatggggtgtaccatttaataatgcagcgctaacataacgtgcgacgctaatatcatttactaatagattttcattcactataataattaataatcttctggccccatggcacaggcaattttaaatccgtacccgtcagccctaaaatgccaagattagtgaatctggtgtcatacaggactaacaggtgcaaaaaccggttgcgtcatcaaaacgcaggatttactcaggatcttaagaaatctaaattttcgcagaatcgctcatcgccaaaattttaggcgtc

SEQ ID NO: 94 - DNA, 2000 bp, Artificial Sequence
  Synthetic
cacgtggttttcagcggttaacgcaatctgcattattggtagaattttacacttaacaaaatatcaccacgcggacaactgatttagcaaatgccgtccgtgacgcgggacccgcagcacattattagacatagtacatcagcctgtaaccgatcagtcatcacatatcccggaaagatttcaatccagttgtaatcaacgcgtaaagttatataatcacttcaatcaccttactaacttcagaatggcagcctaaaaatctgatgctacgaaccgcatggtgttgaataaattcaatagaatggagctcctggatatttcacgacgccgggacagaaatagtgttatagagaagaaggcatgccgttttactcgattcgtaagtagtttgacgaagcaaaaacttggggaagaacttatgagttagccacgacaactaccgggaggatttgcttttcttcctccatgccaatcttggagggagtacctcaatcacacgatgaatcagccttaatgggcgcccaaaacattcttggtgccagaaaagcggatgcttcctcgaatgtgtaatcagaaaagtggtagatgaatctccggctccatcatggatagagctgcaggtattggtgcagcaggaacgaaggttctaccagtaagtaaagtttgacgttagttacgagtctagaaggcccaaagggcaaccaaaacgtcggcaccataacatctacaggtggtaggctaatgtaaaagtggttataattgctaggcagaaataaggccgttcattgggcatgtgtacactccattgatggagcttaattcctctcaaaataattacattctgttaacaagaaataacttattggtcgatctacgagctagcaataaataatcatgaccaaagagctgtgctgtgatcagaagttatgacgcttatacagagagcattgtaaagggcaggccgaagcaaattcacagagtacctgaagcgaacaaaggaagagacttctttataatttacatcgcttggcaattaaagaagcgaaacacagttgctcgaatcacatccttacgtgtcgtcgacaatatcataagcattactagtttagagaggtgagatatcggtagtaggtattagaacattctaatacctaaagctcattactattagcacctttcctcaccttatttggatttcccgcacgccgttcgcaccgagctaagtgcaataagccatggcgatgacttagatgtcacattgccccatgaattcaccccagtgagttgagacgatttgaagtttaatacgtcgttcgtggacagcttgaatgtttcacacgtggtaagttgcatatgaacatataggaggggccacaaagcttatgcgtgaagcaaatatgattcctccctcgatccgttaattagagttgctgaagggcataaactttagcgagtttgtattaacatagtcatatgaagtaacagagacccgtcataacgcttgaaaacctgaactcagaatgcgctttgtgtaccataggcatataccccacattacggagatgataatcgacaaatgctccaagaagtagacctctagccatcatcacgtgtctctactgtattctccgaagttccggaggccagttcttaagtaggcacagaacacacgatggatttcctagggacgtacgtatgttcgacttctcgtcagtaatcgcgacagaaatgggaaggtgagcttaacctaacccacatttttgtcatgggactctgtgaatggtgtttcttatgaagctatcacggtgtaaagatatctagacacgctatgtgctactccgataaccctacgtttaggtttacgagattggagaaatatactttattaattcttccctggaatcgtaccaacaagttccaaaatggctctgcggtctgtcaaaatatgaagggctcaacttgacaggacgactgaccggaaatgatttaagtgaacctcc

SEQ ID NO: 95 - DNA, 2000 bp, Artificial Sequence
  Synthetic
ataattatcgacatagatgtgcttcactcgatttgacagctggatagtaagaattagtgtataacccaatacgtatgctaatacaaaccctggactgatttgaatgtaatcctattcataatattttagctaccgtaaatgtattctgcaattgaatttcgtgtgaatgtaaaaggtttagaagtttcctaagttatcgggtgacgtttttaatgggtcttaccgtagattcagacaatcttttggaaaccaactgaagaaggaaatcacacgacctggcggataagggtttgtaattcgcgttaaaaaactgacgtttgctataagagacgttaatgtaaatgtaacgctttaaattctctgtgcgagagttttttaaatgagatcaaggattgttaatttcaggaagctccgttattggattttgccttctcattcgtcactatccctctccgatcaatccgattgagtcctagtgtagaaagttcacatagaaagcagttttccgattagtctagcggggtactaagtgaacactagtcagttggtgatatactatagctaggctgtgataatgttaatcggtttgtgcctactggaatgcttaatttcatcttgaggacttgcgctaggaatcggtatgtcttcgttaagtccaaagtgccttttcgacagatgttggattgatgcactcctccgaaaaggaatcaaattgggtttataaattttgtctttgtgacacctgccgaatttagatctcaccattatccacaataaccctattatctttacctacttccgtcggagcttgattatgaatattggcagaattatgtaatagtcattaatatgttgaataaagatatcaatacattcagacaattgaattaatcctgcgtaaaaacctacttaggacgagttgctggtatttgtttttataatggtagacatgagggacatattacgaacctctgtaagcctgttctgatgtggccggcgatcacgttacctgatgagatttatagatctcaagtcggatgtcctctttaataaactgaaaaattgacgactaagtgggctaattatgccatcagaaataagctaaccaaacctctaaagtcgaccctgtagtataactggcagtgctagatatcacagggtgtttgtctactgaaatttcggcattctggtcacacttattgccgataggttctagtagctagtttatctagactccaattgaaagcttacttcggcctatcaggttgaatgatagacggtctgtcttaagaaactacaggacatatactgcatcgaatgcgtttaaatcctaacgcagaagggttgttatctgatcatcagtaagcaccaatctgcatgattacagacgtaccaacaactgaatacatcctgcctcctgagaactagaacctattgtattgcggatgagggtaagataggtagaaacctgctgccaacttatcgataataattatgaaccatgcgtgggtgttgatatagacttaatatgacctcctgtctggttcatataccagttttcaatgcttaagagaactagcttgtacggagtttttttaatacaagtgctaaattaacaattgttcaaaaacagtttatagtagtaaggtattgtaccaatcgtatagcaataaatcatacctgtgtttactccatactttcttgattatcgggcacgagaagaggacaactcccaaacatcaatgtagccatagtgaatgaaaaaagtcggttatgaatcgttagctaaatcgtttgctccaattaacaaaactataacctaaactggtgaacacatagataaatgccaactcgttatcgtgttatgctatagatccgaatttggtggttctccgagtctgtatcgtttttaatcgagatcttaccttattcctaaccacatttcgtaagcctattgaaacgggtattgccggttcgcccatctggtagtacgtaaacga

SEQ ID NO: 96 - DNA, 2000 bp, Artificial Sequence
  Synthetic
gaggttagtgatcaagcgcattagctttttactgcggaacgcatacaggatatttacgcttaaaaaggtggatttcgtatttattaagtattctctttactgaattattgtccatcagtaatcgctggctttatgaactatcaacattcggtgttgtgttaagttattaatgacacatgctcgacgttccccaattcccgtgcgtgatatattatcatatgaccattaaatgattaaaggggcataatattttgaaataacactattaatttgaaacttttgtccttttcgcactacatgttggtaacatcgcacgcactaaatactgacatatcgtgcaccatgctttctaatagcactccgttccagtccatagctgagactgtcttttcggacaacacaatagataagagtctatctctcatcaaaactgtaagaaaagctctaccataattggggccgaaacgtaatacgattattatgatatcgctcctgccgaggtcaaacaccatagcactcaaaaatggtatccaatttagaggggctatgagtagttaaaaaataggaattaaggtggcaacaggacagaagtcaataggttcccttgaaggctagattaacagaactgtaatgtgactgcctgtaagcgcactggagacatcaagtattgtacgagtataattgcactttggaggtacaacatcgcactcgactctttcatcgatattttttcgtgggtgaacttgagttaaagttgatggtcccattcacaacgagcggttttcgcgatgtaaacgccggccaaagacaacctaacgccgaattattctacttcatatgcctaagtaagcccgttctttggagaagtctcatcctctattattatacatagttatcatattagtctagtcgccaaagtgtggtttctaattgataaatataataagttaaaaaatgagagctcaaagtttttccttaccgtgccgcacaagtaagtagtctcaaaaggaccgcgtagggagggaaaatttaatgagttctaatataatatgcaggcttgtgaaagctgacattgactactctggactggtcggatagttgctagacatacctattgtgacaaactgacccattatcgagtctagtagaaccggtccgtacaattacacattcttcgtaaactagttctataaagactaaaaaaatctatatcacttggagaattatggaagatgagtcaactccgaagtgtggtcaaaaatattacagattgtatcaaatcgaataggccgtaaacaaggggtatacgttcacagtacaaaataaatcaaagccttcaattatatcgagagattattacactaccgctgctcttgactagtcaaacgtacctctcattgacaacattcagcatgattattgctccatgtcaaagactccgtgttcccattagttttaaaggcataatttatctcttttcctcttggataacgagagataattagacaatgctagtttcaccaagcccgactcgataagtggcggttttagcctacccaatcgcctaaatatatcaaaaatgacttgtacgcgataatactgctcgggtagttaacggccaagtacacgctcacagaacaacggttgtaccgcttatctaattagggaatgtacggctctctcactaatatgcgattaatctattttgatttttatgcagagcatcctaagtgaaactctagatgccgccaatttttgtttatcatttcgccaaccgtgaattccaagatggcccgccaaagggcgtataaatcgagtatttacgaagtaataagttaattctaaaattctttaaatatgaagacaaacaatgaattgattatgatttccagatatttactttggtaccggattaaacccatttgaacgtcattcgatatcaaagtccgctaataagggtttcaattacaattcttcaggagaacacatcggtaaccttc

SEQ ID NO: 97 - DNA, 2000 bp, Artificial Sequence
  Synthetic
gcgcaaaccagcaaattaggtttgaccttcaacaactgtaactcgatctgcagacgagtgagtaacaacagctactggtacaatttttttgtaccgcagcattcaggtattaccccttcacgctcagtacagaggtatcgggcatccgtataaaaaattgacttctttttacgatagtccaatagaccgttagcttctacttcatagtactaataataacctaatgcaatagtctggataacattcacgggacactgatactagaatcaactacgctgatgagcatgtccagactgacaatcggtcgacatgagaaggaatagaaaaaatcctaccctgttaattctggtcatgtttgctggtctctttcctactcggtgcttctcaaatgccacatattcgagcataatacctagttataggcataaacttattgttgctgcccatgttgagcattttttatatttaggccttttacgaatttctgtttctattactaaagatgtcagagtaataccaccttcagacagaatcacatgattaaaactatagaatcggcggtacaaagatgtatctcacctatagagtatgctgataaaatcatagaccctagacatactattcttatcgccccttagaaattattgtaggggttgcgattacaacgcatacggtatttgctatatgagcactcatggcttatgtgtacaatttattgatatatatatttagagctccggatcgggttacagaatcacttcacgacccagcaaatgctaatgatttaagcgtagtatattggctttgtgtccagttttcactacgggttcctttctatgtcctgataatctgtacaaccgacataccctgaattcatgccgcatatgtcgtgttaacagtgatctagggtccagtgatagggtcattttcgtatcgtcgcatctgtatcgattggaaaagaattatacagtccgattatcacttagaactacacgaggggacctcttatctgccctacctattggagttaaagttctaactgctcaatctcaagacggccgaagatggttttaaaatgacggtccacacatttacagacaaattggaatgcttagatatatcctactgttgatttttgtccaaaattagaggcgatgtaaccccactgaaagattgagcagtacagtaattctaacttgaaaaaataaatttttgggtatgctcaatctttaaggtgacctactaacaatatcctagatcccatacggtagttcgacagagatccaatacattctaatcgaacattagtaagttaaataatatagagctacatttctaagtaaatcgatgcttgaagatattggtagttcgcagaatttgcatccatcacaaacactagtctttacgtttgccaattgctaggtagagtagattacgagtcaatcagaagaccaaattttttgacccataggatacaacacgtagtcatgacaatcgcatatcgctagtatgttagatctaagaaaatagtctacttaaccgggtcatacatctcagctattaacgatattatgttgccttatgttagacacgtcaataagtagagcatgcatttctgcctcaaataacaaatttgttaatatgcaatgaatacctgagttgaatgaacccaaactaaactcagggtccttccatagcgagagcgctaggctaacatgagattctgacgtcttcgtgagttgacaggatcttgccaacaaattacatatttgaataggcatgtacgatccattatactatgagtgccagagaaaactctgctggccgaccgttttacggggggaaagtcaaatatgtagtaagtacgaattttcctgggagactatagttgctgaacgttcttattctcattttcttgaagttaaggatggtaaaacatactatacctatgtagatattctttggtagtataactattatagtagcgtagacgttatgtg

SEQ ID NO: 98 - DNA, 2000 bp, Artificial Sequence
  Synthetic
gcctaaagacctctatattttaagctagcataaaggcaggagacgttctaacatcgcaccgagttcgactatgaagagaggtattatcaaccctgtctcccagttcacaccggttgcattatcatgacgtttttgatttgttttttttgagtaacgggttcattgtacgttcgatagagtactcgataaacgactcattccacgcaagcctattttgtaacttataactagacattagtctatggctactttcacacccgaacttacgaacaacgagtattttttttttggcaaaaacgtaacgttcgtatgtggcctaagtcattaaaagacaaatattgaagaaaaacccatgatttaataccgataggacattacaagggtcattagagataacaaataaattaggcttcttccaagagttatccgactagttgtgctccagatctgcgatactgatcgaatttatacctcattagacattcgtagtcattggtgttggacttgaagttctgtacaatcctcggtgatcactcttggacaacctgctgataaaacatgtctatcgtcagtccagtttgtataataaactaatgagacaatatacaaaacaatccgtggcactacatgttgtataccaacataaattctgaagacctatgattcttgtggccgaatagtcaacagattttacgatcactaataaccatatatctgttacttgtcttctcagataggagcggactagaaatactcacttatgttattcttacgttactgtgccagacgagaggtttttgcagactctatggtttgccggatcttgctaggaaaagggtaactggtgcctgattgcatgaactatgtggtatgactatagatgaagcatccgtcactgagctcttcgaagtcttttatgagacaagaatattctttgatagaatcatctatgtctcaatttaatcaagggaacggttgggtactaaatcgagttatcatgaggtcctatcggaatgcattgtatttgagcaatatctataactgtaggtactatggcggatatttattttccttgctgcgacttcatgtagcaagtcggcaattccccgcggttttacattttctgcttcgaggtattaaggccctaaagttgtatatattataaattaaagatctggattattaactcagtgcagagggcgtaatctgacgtggcgacatgtagatgaagcttgcccaaaagatatgagatcttaatatctataagaagtatgcctactgttaattttggggagaaatgctaccccggacaattatgcgattgtcaagcgaatatcttgattttatccttggaataggtatattacttcggttacaccagatatgaacctatctattacttcatattttactcaggcttggtcgggacctgtgttactttaaaggcattaaaacatacagcgtcgacaatcctcctaatcaatatcctcagaaggaatttactcgcaatagcgaactgagttttttgcctgtacaacggtcgtgcctactcaatcattgccgcatactaatctctatcatattgcctttacggggcgaccaaggaggaatcctatctaatcccagggcacctggaacacctgcggaacatgcttcaataataacatcgtataagtctatgtctgcgcttgtgacgtcatagtacttcttctagtgatatattacgccgttggattgggatcacgtttagaacgacactgtgaacttctatatgtactcttttctcacgatatgccgtcgagttttttatcgataataggcagtgttggagcgggacgtgtcattagtaataagtttttcctatcaatttcctgcgatacttgactcctttggggcaaacatagacgacggttggagtcaaggtgaaccaaaatagaagtacctgggtaaatgcttcataggcacttggacaagacattaagtcgacacactatgccttt

SEQ ID NO: 99 - DNA, 2000 bp, Artificial Sequence
  Synthetic
aatgttcggtcccgggtaagctatcattctataaaagtcccaccccgcttatttaagattcacagcgccgcaatgacgcggaacagggttgtctatgatgacctaactacggcactttaggtatcatatattgagttgagcgaatggatctgctaggcttcccgtctatcggatgctttaatgcaggttaatggcccgattgaagtttatagtatatatatacactgtgatggtgtaactacgttacttcgttactgatcaattttcaaattatctcatttgttaggctacaactaggactaaagctcaagtaaccgatgcgaagaggccgagatggtataatcaacgggggtgtaatctaatatacgaatcatgctaggagagcagcttatcgtcaaaactctgttggccagattctaattactctttattgtatcttttttcatgtagattaaccgtgaagacagtagttcatgtacgttagtcaattattgagaacattagcttgaatggacgcgtgctcaaataataccccagtaatctaaaccatattgttaatcttttacaagacccaccaatgacctaatgagttcacctccacatacctgtcattaggtgaccttatttccacatttgtattaaatactaataactgaccatattgtgctgtggttctgtacacttgtatacctgttcggctaatactagtcagtgatttcatagcgaatataacatttgacaagactgtagcaacaagtttttggtatagggtttgttaaagcataccgcgcaggacgaccgtctcttacattaatttactcgttttaatctataattatccatataatcaactagtcctgagccaaatcttcaatttcccccgcgtttgagattgcttgatgaggcgaaataagaggcgaacggaactccaaaaaagagcgatcttttatcacgtccctccataacgctttataagtcattagtcggcatcgttacaaattaatgatagaccagaaagtacacagacgtgtcttttatcctgtaacgaccctaattcggcaccgtctactaaatgctttgccgtacgctctgatgattctatccagcgattacgtatatgttccggggtaactacctaaatctaatgcggccataggcccatactgatccgccgatttcgcgcactgctttacttatatacatcagtactactcgggcaaccggtaaataatttacaatagaagtttaagtgcagttacatgcttaagatatcgagagaacttgtgaaatacgtacactaggattttctcaaattcgtgacattacaaggtctggtttcgcgattctcttggactgatataatatgattgaaaaatgtagtagatatgatcctggataacatttttaaacaagtcttgggtgagctcggtaccttaaatccgatcatagaatacaacatggcacctacattcatattaaatagtctattacatgataagactccttcatgtctgaaacattggttagacaattcgcggtttcagtgggtagcgtgttctattgacttcgaaatgagaaagtgtttcggcgcgtacggtatatcttcccccatgattatacataacatccttctaaaaatcgcgccactgcagggtcctcttttcttatatattattgaggatttggaccgatcaaacttaatattaaatatgattctacatacaaaggtaatgatggcaatctacttgcgggctcgactcgtagtctgttcaatgaaaaatacatttctcaagaaataatcttcgagctatttcactctgtagttaaagtttcaatcttgttacatactgcttatacaaatttaatttaaaagcatgtgtcaatttaaggctaaatgctcagtgtaaattgtattggtaaactccctaagactaatgaataacttgataatgtggatagattaaatccgtgcaagcctatcctaaaatcaatttgaagtg

SEQ ID NO: 100 - DNA, 2000 bp, Artificial Sequence
  Synthetic
tacaaattgtccacgggcgtgaaaacaagcccattcttcttcaattgcaagatttgcgatacttaaaccttactgatttaataatcgattcaaaacgcaagagtcatgaacagaacgagaccccgccatatttaaatgcacattcgtgcagcgatgggtatattgaggctgtgagaggctcaattaaacattttaccaggagatgggcaaaataatgcgtggggatcgcgggactataatctaatcagtcatactctaaagtgagcttcgtgatatcttgaggataaaaaagggcctaagcgcacagggttattgagttccagctaatgatgctcgataataatcggccgtaacttcaatgcgaagagaatatacgattctgaacagttacagataaggcctattaggcgcgaaaatagtcgtctaaaagaggagaactgctggtcgagaatgagtgggggttattctaacaaaggtagctaggtgtggttataaacgagaaggactacacccaattgatctcgataatagggcgggattgtttattgacagtagtgaggtgttctaataacagaaatttagttaaggtgcgtattcttggagtagagcacaaaacccgctaatgagcattgtatgaatccgcgacaaaagagcaaagatcacagcaacgaaagtctaattgaaatagtcctcgattatgccggtgagttgaaaaaagttgtacgttcgtttatgccgttctagataatttacacatcacattcctcacgtaactacatgatttacctactatcacttccaatcaccaactcggatttaggaatactgtaacttatttccgattatccgattgagacctaagcagaaaaacataagatgcccatccgaattgtgatgtggataccagttgtgataattcgtcggattgaactcagcctgcttaccgcttttgatcgcagtcgccgcgggtagatgtagttagcctcaccggctggatacatatctccaggaaatcgcggagtatcaatctctagagtaaatcccctgccttccgttgatcgtcttgctcacctaaatgtctgaactaggctgagaacacaaccatactccggccacgtagacgatgctgaatattacgcagctatactcaaagttaaactcttctcagtgatttatgatgtagcttagtgatctttacagatttggtatcgattgggaatccagtttaaaactgaaacgacatatagaaatatgtaccaatctaccagcgcaaaccgagtcgaagtcatattatacggtaaatcaccatcgtgtgatatattgcaatttgaactgatttttaatccctagcttaaatacttcattgatttctcgcctttaattctctgaacgttacaatttttctgcccaacggtcctcctctagaatacctcgagagccgacacaaatacagttagagaatttttggtgatttgtgcgacttattagaaccacggggtcatgaccttagcccgaataggtagtatccggatatctgaaactccaggcagtaataatacattgccggaacgacaatcggatctagtgaatgcgacatagacggtaatatgttaagcacctcatagatgattactatcaggaaatatcaatttaaagctgcgatgaaagggtcaggacccagccctttcaagtctacgtaactccactagccacattgtctaagggtgccaatcatagatcatgcatcaacaccggcgatacgcttgttcaggcattcatatcttatagttataaaatttgtttatcgtgtgcaggggtcgatttttctcactttcggcaaccaggaaaagtagtaattactatataaaatgaaggcgaatttcggattactctgcaaaaaatcattagaatacacatctaggatccggaggtatctgcctccatgaagttaactccattgtggatatgatgcgagtaacatatttaggtccgaagaaagg

SEQ ID NO: 101 - DNA, 2000 bp, Artificial Sequence
  Synthetic
atcatctacctaagacagagctgaccgtatccattgtcaatagaacagcaacgattttttccatcgctggaagagtgatgcgcactagttcatttcggacaagtaacttggacgcgatacaagatacaatcgatgtcacagcctctttagtacataccatggaattatgaatcgactaaaaacgcagacgtataattcagctgatcgaatgatttcgattatataccgaagtcagtgacgagaaccttcactttgcgggataccgaactctgtcacaagaaataagtataggttagaatccagagaaaacattgaatattatgttttttcgcaccaaaataatccaacgatgttacgcttagttagtggatatcatgacttcactaaacacttggattgttatctaaagtttttatcttcctggctgcgacattgtttatttaagacgtagttaaaaaagtcgaccacggaggaggaattacatcgtcgctgatgagcccattttcgctaaatgcagtcgactacgaagagtttttcgcgtatcgtcaacataagttgatctttttagataacaaacaaaactcttcgcatcgacgtaaaacatttttcataggcgctttttacaccgaagaatctcagcttcagaattgtacgatgtcttgtcacagatatcctttaaacaaataactaatagcgttgattgtttgacatctactccttattgttatgaatgtataccatattgttatatgctattaaatcccacatattgcggttcgcactaaaatgaacatctatataacttgactgttacttgaattagttatggtccagctaatttttcattctaggcatttaatcctttatgttccatagtttccttcgacgccttgaacgatgggtgcgagtccgacggactaacatttataaacacatttgtgggtttgggtttgctacagatatctggacgcaggatgtttagagtaacatctgttgtcatttggctagcaaaatttgagttacctgatagaccttcctcattcccttaatattaaactgtctttctcgaataccgttcgcacagggtccaggaaatgtgatgttatgacggcgtgcaatggttagtccttatgcaggagtttctccgcacccatcaatgccattattttacagtcaaaaaaacataaacttgtatgacgaatgcagacctttgaacttttgttaacctacttttgtaaaaccagcgaaccctaacagttatgtaacgagatccgttaaccaaaagcggttatccgaggataagcttcctacgacgtcacatttgtcatcttccttaccggtatgaattgtatgcaggtccctattcgaaatgtggttataactgatgggtatcagcaggttatttataacgcgtactttatccttgtaggttagttgctcagtacgcccaaatcaaagaggaggccgaggtgcaggaaggacctgactgacaatcgtaactaaattatccaacaggattgttaattgacaatgtttacactgactatggcaaaaattgtctcccaaacggctgcggacagcgttctttttatcgatctgaggtagcacttgcatatggatatagcaataagaaatagggagataccagcgaagaacggagtagatgcctgtgacgtgtgccgacctgacattgattatcgagcatgcggattaaaattcaacaactattcccgtgaagagtgccagcctgtagtcaattattgtggatattatctaagttcagatcatacctctcgtcggtgaaaacagatagaggccaaagggcaaatctattgaatgattgacaatttgatcatatacgtgtctaagaattaattgtaacggatgcgaattcgttaatcttcctggggtactcttctccacgtcacgagagataacaacaacatcaggcttctgataaatagcgtaacaacgtattatcaaatgcatcctgtctgtat

SEQ ID NO: 102 - DNA, 2000 bp, Artificial Sequence
  Synthetic
ttaatgacccctgccttactgcataaatctcctaattgtgtaatcactcctcactcagataacgctttacgtatggattaccaagtaagtgaaatcactatacaagagattgcctaattttgctaagttagcgttgttcgtgttttataattttattgtgagtctttcaccgaagtagaaggaagtaaactcgcagtttcttataaccacttctaggcgatgtagacgacatagaaaatggggtaaggaactcataatttttaagtcaatgatacagccttaaaagataaaaattagattaccgtttaatgagggtacgtgaccattaacagtaagaaagcctgcaagcatgggacaggtgctattgcagagctcataaacgaaatgtcgcttgggcgtcctgcaccagatacttagtggcggatgtcaatagcgaggacgaatcattggatgaatattagctagtggatacggaaaaacgtgactacgattgcggcatcgagttcttaaccctctcatggaggcatctctcgaccttacacagtgagagtgcattttgttcgccagtctactatgacacattaaggctcaaacacgctctgcttattcatttggccttggggttctagatcacactacaattgccctttgcaagaaaaacaaatgtcattgaaaaattaactgctgtcttataaacctaaactaccagatactgtaattggttttaggtttgagcatccaccaacaccaatagccaagattgttaaactctaataactgtctaatacacgtgcatattcatagtgaatcagtgcggttcattttctgaagagctccaatctgaacgatacaaggcgtcctgcgcgtggattaaaaacaacttaagcgttacgcagagcagtattccattttataatataccgtttgccgcaggaggttatattgtagaagattagttcattttgtgggggatttacaggccaatatttaccaaattttacgaggtagttgaacctagtgttacttcgtgaggctcgaacggtcttcccgctccaactgtacctttagatgggggcttctttggatgtaacgaagtaccggcttaatatgagacgtttgtacgcgaggcattcttatttaacccatacttaatcaattcaaaatttatcttggtgagtagcactggagaatttggtatccatagcggaccgatagaaagattgttataccaaaattcatgaatgacgcttagtattttctagtttgataacatggttaagactacattctatccgaattcttattaaaattgaaatgacgcattgcatgctgtgattccaaaaccatgccgacaggaggtcttcttaaaaattcagcgtgaggttactacaccttcaaaagtgcataattggtggacaactaaaggataattgggtaagatctttctacattccattaaaaaattctaacaaaccctatctcatgttaagtacttatgttgcctcttactacattgaccctacactcagatatgataaattgatgtttaacctaactatttaaaagctcaataccttcctttttacgcgcaataaaaggttaggcacttttaatgtgaaatttcagcgaaatattcgatcttgatataactaagtttacagttcctattactactcattataatagaatgtatgggctatgaataataaatggacccttagaaggataaatgcattgattcgatgctagagtaaactgatggctcagacagaatcatgcccatggggaaacataacacctaatcagcatcaactaaaagtcacatgtacgagagcagaatcaaatacaaatcaattatataacgtgaacgtagaatccggaccagggacgtttctactctgactatattaccgccagctgctatagtaatcgcgtatggagcatgtatttgctgactaatgctaaagtacaacattactgtgtaatttaaaatgctacct

SEQ ID NO: 103 - DNA, 2000 bp, Artificial Sequence
  Synthetic
tgtacttgtcttcttgtttgtcacatacggaccctaaatgaccttgtctagttatccgatacaccttgcttaagtagcctcccctagggggaacttattacggaataacagttttacagtattaatcaaactcttatccacgttttcctgtgatcacaacgtattgtttcccttgatttgttgagaatctctattgagccttttatctattagagtctccgtcgcacataatcccggtgcgttgaacagatactggctagactccttacttttctatcagttgaacggaggatacgagcttcaaaataatgatttgtttgtagatgtcagagcatcgtcgtgagaggaacccggatagggggaataacaggtagcgttgcggttgcctgactaaaacccaggactcaagtttcattattaacattatttgcatgaatgacagtgtcgcagatctggtataatgaccaacgatcgtttagtagataaattccaatctaacaaacactaaccagtatctcagcccacattgcatcttgttttagcaatcctgcagatatcagaaccctcctgcagtgaattgactagtgcacgacggtaacatatctctttaatagcgcaccgtcctcaacgtagatgttacgtctggggttatattgggccggaatgtcctgggcttggactaatgaaggcaaaggctataaatgtgcttattatttacttctgcgtacttatttggagaatgtcatattaaagatgtcgcggtggtcggattaattgaataatgtgcgacttggatgcacctcaatcttcattgttttgaaaagtctggagacgtgcaattacactctatatgtctttgtattaatcgttataagctctaaaggagatagcaagctcgggcaaatggtagattaatgcttcaagaaaatacaagcctggggattcacattccgaatatacaactaatgacgctctcattctcttgcaagtatagtaatcggcccgctactctatggggagtatggcatcaggagagagtatcattgacattcgaagtttgcatactgagcaataagcgggtaatgcttcaaaacaaagtgcactcacttaatgtcggacattgtttataagtgttagcgctcaattttccgcaatcacgctcgagcactaatagttggagttcgctttagtttgataataacaaatatgactttgtcgcgagattgcctatttgcatccaggactatcgaacgcaacaaactcgtgaagaggccgcattttaactgcaggatagtaagatctaattatgaaatacatagtccagaaaatcattcgagactacttaacaaatagtttcagaggttctagactttctcaaatgtatgtagttcgtgaatatgtagttatactcaattacgactttgatttttatttaccgcctaagaaacttgattgaaataatctagaagcctcaatcctgctccatcacaaacataatatactgaaagctagagggcgttaccacagtggtacgtctagattccaaagcgtgctaggagattagtggtcgaaacgcaggttccgcgagcagtatcaccctacaaagtagctggttacagtcaacacctagcagcaatttcttcacttttgttacgatacgtccgtggcatgatcgtcgttgcctaattctacgacttaaagataccgaaaaaagcaaaatctagaaccatgatagagctacaaaatccctctacccgttcgtacgtgcttcctaatcagatcaactatgtgagcgacatagttttagctagtacttgagcgggagttttgttctcgtctctgaatatataaagtgtttaatgaagtgctatgagggccactcatctttagcatactaaatcatcagacataaaggtcacccgaaataatcaagcagaagactaacagaacatgctaagagaggtctttcaactacgcacttgatagataaccgttagctc

SEQ ID NO: 104 - DNA, 2000 bp, Artificial Sequence
  Synthetic
tcacgacgagtgaggtctgagaccgtcatcaaagatcgtaacactttttaccgggctgccataacgtaagatgcatgactgcaagaaagttcacggtggtaatttcaatgagtcattgtcattccctgaaggacgtataatactatgttacgtagattattagggatccttatgcgttgaggagatatcttgccttgagtgaaagaaactcatctgtttagaaacataccaaatatgtcagacacggtcggctttgataagagtccctaactaattggctgcacattacgattcgccgaaaatatatgttgggagtagtgtacacgattttagacaaattcccgagatgatgaccgtgacatgtacaatcgcactaaaaatccccggtattagactttgaagtggttttggtatgtgatcttaagcatattcactatactagcataacaatggtggttgcttttggacgcaagttctgagtatatgactatgaagcggaatcgattaattatgtcttccaataaagcttagaagtatggttcgtgaacagcttccagtataatttagagaggccgacaatatatatagggttttatttactattggccaagaacatcctcagtcgatctaaacttcttccaaagcactaattctatcgcaaaatggtattataacaacactaatcttggagtcaactcatatacgcgcgtgtagagtcatgtaatactcagcggctaactacatgtattatgtcaagtcttccttgctatgaatactggtattcctttgtggattaaaacggtaccgtcatgtaattttgagataaagatctaggacggggaagaaaatagtaatacggtatgtatgcgttgagttgggtctggatattcagtcaactatgggtaactgaggactttgacgctgcatcccctgctggtgcgtagtcctaaaaaaaattctctgggacaatatgtcttcacaagatccttgtgagaatcccgcttccggtccggctgggccatatagactcctattactttcaaacttcgcacagaatcttaaatatgagattgtaaggaaactatcagatctgctctagacaccgacggaggagctcccggaacgttccaaagcttttttttctaagtgttgcacttggccggtcgtacacgcagagcggtagataacccaaatacagttcttctctatgtctacgcccattatgggacgcgtggagtctctgtgacgttgacggtttataggttaagtatgcttacggatgaatattaatgaatcgtcgtagttattgaagacggccgatgtagtatgcaccgtcagccgattccaaactagtatcttgctcctgagttactctgttagattcctgtcagtttatccattttagtgtagaaatatccttgaatggttgtaccatggctcctagaactagacaagataaaatgttataccgtctggtgaacatttaacctcgtacttatccggactaatggtaattgtcgaccgcctcctgaaaactcgcattggtgtcgaaaaaagcaatgagcgcgtatttttatggagataggtgcatgtattagtctgtattcttagatgctctgtcgataacatgatgtaatgcgaattgattagaacaatctgagaggctgaaattgattgcctgcccaaacacgatacggttcgatagctagctgccgatgcgcttcgatattaaacgtaggcaaagacttccattctgttggtggtaatcctatcgattccttaatgaacccacgacattggatattgatatcgtgcttagatatttgccaccatatgatgtatataattaaaatacatatgcttaaggcgatagtatttactccctgtacgcgcagttaccgttggcatgtaacaatttaatggcccaatgaagcgactacgaaccatataatttgctacaatagtactattaacatgctatgaatttatgcaaaaaaaaa

SEQ ID NO: 105 - DNA, 2000 bp, Artificial Sequence
  Synthetic
gagttgattttccgcatttcatggaaatataatagggtaacgtttagttacggaacgtattcttttgaaaactctacttagtgtcgcaactaaacttctctgttttagtacagtcaggattagagactactaagaaattcctgatctgctcgctactgccacactttacgcaggaggcttgttttcgcagtaaccggtgagttaaggtccaacagggtcagatgtcccttttgtcaccacgaatcactggctcattagaaattgatagatttgttaaaacgaacctctatgtcaacaaatgcttggaacgtcattatgacagtgttttgatgtcagtttatccagaagggcgagagggtcatggcgcggtcaattagaggttcgcatattagtacttaggtattgtcagatcaccggagtttggaaaccctgcttgtgtgatacctacaacttaacttggcccaacatgagaacgttccatgcttctggtatccgtgtttaagctctcagtggagaaattcttaaaatgatattcgtaactaaaggcatgaaacaaaatgtgaggatcggttataatggacacagtcctgaccccttcgattgacctaaaatattgaaactacattcaagtagcgagaattttttaattgttcctaaagttttattattagataagtggtcgatgtgtaggaaataagagatgataagaaaaccagacgttatttaaagggaaatgtccaccagtgccccagcgttataacatgatagccaagaatttggttatacgcaaagttcgattgcgtgctcggttactggagatcaaattaatggagcttcaataatagtactaaatcatgttttcaatttcttagcacatccccactaatagtttgtctcagatattatatgatatagttgatcgaccctgttatacgcctaaaaccaattctctttcgctacccgagagtgaaaacatattcaaagttgtcagcctcgacgtttaatcttcgtaataatttgtcggtaacagattaaatacggaagacaaatattattatcttcaactgtccaaattctccgtctccatttgagacttactcatacttcagtgaccttggcactatagctgatgtttggagagaattaaaccgagatacttataataatgagagctaatgaaatggtagttcgtatatgcggttatagactgtaagaactatccaacagactctgccgcactctcagatttcatcttaggctaggttataatgtatgggacggctcggatattctattgaatttaacaatttcgtccaacaacccttggtaactgagtttcccgattacatgacgatccagcttaccgtaaccatagaacttggcaatcctctccttaaggcgcatgactagatcatcaatcgcacttcttcaatcaagttctctatctggcgcggacatactgttttacgtctcgtttcattgtaaaaacccttctgtgtaataagaacacgcgactttgatggttgcgatccctacgtaacgtgcacttaactacatatacttggtgagattgtgctccatattgaaagtcgatgttaatcaagacggagttgtgattaataaaatggcataatacacctgtgtttttcctatataatccagagaggaaaataactgttttccgaccaagtttgtactagatttatgattttccgaatatgcatctgcgtgagtgtgtacgtctgtgtgcatacgtcattcagaaagatcttccgtatgtgagaccttttggatcagttgttcatttttgtacctgcctactttagaccaggttctaaaaggctcatttaacacatgattattatagatcatataaccattactcctaatcaaatttgtgccatcgttgcaaccgaaatcgtctagcaagatgatcatcgagcaataccgaccctttatataggctcaaccctatattcagaggaaaatcacggtttgtc

SEQ ID NO: 106 - DNA, 2000 bp, Artificial Sequence
  Synthetic
gtccatcattgactctgttttctcgaggaactctgcaaaccagataagagattattagcatatatgtacctagaaggacatattatcgtggacatcccgggtgtttgctatttgagatttattgattgttttttggtaaaagatctgatttacatggcattatagccgaggctcatgtttacattagcatagtaggctggactagttgcgagagattttgttacccgggatcaattgccattacatcaaatcacgtgaaacgcttttccaatacatgcatatcccagccgatacttagtacgagatgatagttgtacgacggatatataattacgtctatacgttataaattgtcacctgtcaccactttctgaattaaaagctgagggacgagccgtattaatactaagagcgtaagagcctcctagggttatataacttccgcactcagctattattattgaacctgcgtacaagtatctacttattcaagttactacgtatgaattagtaagcatcttgttttacttatgaccgcaatttcatacgttgcatgataagacaagttcaagcacaataactacggcagtaggaattgtggctcgacaagagagagctgttttcgccgttctggggatgagcatatttaaagttgtttaacacatcctttaacgataacaaaagacatacacaggatgaggtatttctgtcaagagaattggtagtttgtgttaagaagatccctgaccgtccttagatggaagaattaacgtccatagctggaggtgttgtctttattcacggaagcataagagactcgtagtacagaataagacggtctcagggtatccaccaggatcaacgccagaaagtgggcaacagatcggaagtggaattcggaacaaacttcatatgtgaaagaaaagctttgatacgacttccatgccttggtgataggtcaaatttagctattagaaactgcaatgggagatgttcgtgcatgggaagtaaatgtatcgaccataatcgctctgcgggctagagcttgcggacagttagcggttctttagacgggctgaaccctatcgagaaccgatacagcaatgtagtccattacgacatatgtgcttcctcgactttactggagaaccttaagacgcgatggattatttaactaaatttccagttatctgaactggcataatttacaacaaacctaaacattttccatagaaactcgttatgagcatttcatgcagtgcgtccactgtgatatctgtaatggtaatcggtcctcatgcgatacggctcggtagtttgtcttgcgacttaaggcaatgatgtgtggcatgctgtccagaagcagatagatcagggtcaagtattgcccgcccatttaattactaaagagaataatgcacataataatctctattgttaatgatataattattctagtgatttatatctttataaggtaagcgatttcaacaaattaaattaaacgccataaatttctagcaatttagatactgtatgggactattagggactccataattaacgtatgacatactacactaataactaaactctatttgacagttgcattgcttaaacacccttgtgtgttaaaccatacaaccttatgtctggctatatttgtacttcaggaccgggattcatgataagtgcttaggaacctagacgatgaatcaagatcaacgtcttatttataaaacgttgacacaatattaatcctacaagatctaactttaccattaaacagaacttgctaatccctaatgaccaacagacttctggcaacgagaaaaaaataatcataatttgtgcggtacactttagcattaatttctaggattcagctagctgggcctagggaacacgagctttacgtggcgtcgtccgaatcgttagagaaacattgtgagatactcgatatttttatcggtagaatcctccctcattcttacaatgta

SEQ ID NO: 107 - DNA, 2000 bp, Artificial Sequence
  Synthetic
ctcaacagcattctatagccactaatcttatctcacaggcgcattgctgccataccgttagagggtttatgagtgtggtgccaaatttaatttccagctattgctgagaagtcatataagtttaagtgcctctattcatgaatctacgaagactacgccgtctgcgcactggctttgccgtcccacttaatttaacgttaatatgcaggtccgggttaattcatgaaatttatacgagggggtagattgtcgcattatacgctcacctacaaatctgcctatcagcacagccattatgactagatttaccggggaattttcatatacacaaaccacactcattttcccacttataggattgagtctcagatcacacttgtgctgcttgctgcaaatccttttatcattgttcatggttacttgtttaactaatatcattcatttaagatagggtatctttataccttgaggccaagttttttcacagaatactgaacatcgaaacctttacttcaaatagatcaggtaagattgtttttcatttaaagcgattcgctcatacagctttctgttaatagtgatatggattggaaactaaattaccgagatatatcgtcatcgtcggcaagcagctgctttatactaggatacagaagacggccgtttccagtaaaaaaaccgccgattcgatcttcgattattacctttttacttgcggcaccaaatgtagctgaattatgttatgagctatgcgtagtataccccctttgtcctagtgctaggctctatcattttatgaaatttaactcttgctccaggatacgtcggatgtacttttaacaaaatctactgagaggacaggattgaccacgtaatagtagaactgataggcgggatgataggatcatgggcagtattgctgattttagaccttggagatagctgcttaatgagctcctcgacctcacacttactgcaaggtcaagataagaaaatctcctaaagatcaaaccattccaaattcgtgtttacataaattttactattatacatcgtaatgttaagtgatttagctactgtgtgtctaggatccaggatagtcgtctaagaagccgaccaacgtgctaaataggatttgaacagcgttatagtttagtttataaggttgtctattttatcagttactgcacgacacatatactctcagagaatagggtatcacggtatacatcgctatcatattgactaacgattgttcacggcttatattttcacgagcattccaatgtggtaaccattcgcaatcatctgggctctcagttgttaatgtagaatttaaccaggttccgtattagtcgaaatcgatgctctatgacctcaaccttcctcttgtcatgatagggtgactaaagaagtttccgatacgcgacgtgaagtccgattattatccagatggtaaagtgaagcttaaaacataagagatcattctctctgatgagacataatgatatcatttcaaagttctgttaataatacaactgctagtcaacggaatcctttccatctaaaggcgaacactaactaatttgaatgagaaagataacactaaaaccgccaacctagtagttacttgagctaacacatatattacttaagtagctttatctctggtctaagtcggaggtcacaatgacttggacttcttttagtttttcgagtacaactagacaatgacctcccgacgtagcatatagaaagttagaacataggattaccgagtggtaatagcccaatcaaattatggtgcgaaaagatagtactgtactcattacttccggtatgggacaaagccgatctatttgtcggagcacgttaattttatgaccggctaccctacgtttactgagtctaaaaatttgtaaatacaaaaatttttcccgcgctaagttaaccataactctcaagttatacggggtaatggatcttaagttcccggaaaa

SEQ ID NO: 108 - DNA, 2000 bp, Artificial Sequence
  Synthetic
gtaagactgattaagaaattacatagggacctggaaccggtatcagatttcaaattttggataataaaccgccaggtgttaacccatcaacatctagtattggcgtagtgagatctcttgcatttcagacatcctgggacggcaggagtttctatccattttccgcaagtgttatgctccaattgacagatatgtcgccgaggaacaccaatctggagaatatttagtcgagaggcacaactggtgttataatcttagtgttatcaagatgaccttttggagtcctttggatacatgaacccatacaaattatcagcgctctactcttctgtaacacctcggaaatacactgaaacagatgtcagagataaccatgagtggtgattgcaatcggtgaccatgttcgtagatcagtcctacgagcgtccatatggcgacgagggaactccacctttcgagcaatcatattggattgagcaaatggtcattcaaaaatatactgttcactctgccaatataaaaatagcactcgttttttctattaggacgatactaagtgggcactttatccctaaataactttcacaaacccgattatagatcccccgtatccaactggtagaaggcggctcggatctatcaagcatttgccgaattttgcgtgaaatttttccactgactgctaagcataaaccgatgaagccaatcttgaatgggttatcttgaaaatattttgctagatttcatagaaactttgattaactatatacgatatacttatgaataacgcgaattacatatatagacatgttctacgttccctgaccttgcgtcaacaaaaatcggttatgtcttaatcagaattgtattataatacatacgtagccgttttttaactactgcttataagagaatatttctatacttactacacagatgtttggactataaatagaatgacatgggggcaggggaatatgtataaatgcctgtgtgatctccaactgcgcattttgccgatgatatgtagataatactttgagtcttggacggccaacgcgcacagactacacactactatagacaatggatgatttcagacgcaataaaatgctaaaatcctaccgattgtcatatttttaagtctatacctcaccgtatattgaattcatgtcgtatccgagcgattttcgatttgccctgagaccatagataaaactcactgagctctaacgtaagattcaattcaatcaattataagagcaaaagtgtaacccgtcgaagttattaagctgaaatagtcgcaaaaactgtcaggtattgctgtccaagttagcggggcgccatgagaatgtgaatgacacggctccttgatatcacagcgtcaatgtttaggtggattagagcagagatataacgaatgctcatccgatatgacgtataaacaaatgagtaatgttaacacttttatactccggtacctcagtattccagatctgacgtccgtggacacagtcctcaattacgctgttattgtatggactacccatcgctgcttgacacgatcttgaatttatatagctacgaatgcagaggttttgcaccgcttggcactaccgagtataaggattatgtcagtcgaggcctgaagcggggactgtgaaaagcactccacacacaacagccaatgtagagccttcgtgtttgaaattctaggttttcaacatagttttttggctgctattctattaactactagctttacttgtaatcttcggctaaagtaggaatgtattaattcgctcaccgaatatcgcccatccttgaccacgatgtcccgtcaatttgtaaaaggcatctagtattcatcacggtatggtatcccttaagttgtgtatggctacaaaaaagtaatggaatctaactaattccatcatgcgcgattcatgagctcgtgtctgtatgaaagaatataccattcaatagacacaacaatgatt

SEQ ID NO: 109 - DNA, 2000 bp, Artificial Sequence
  Synthetic
caagctagtctaaactaacaacagcaggagggcgagaacgttggccacaagacattaggcgttctgtttatcaagcatcgacgtctaataattttaatactaaaattcgtcactatctagttgttcaccatggatttttatgtaggcgatatcaattcagtaaggtaaccctagttctctgggctcatgtatgaaatcgggaagaaagatatgaatgaaaagaacctaactactgaagggtagtcgacgagaggcagctaataggcaacctttgtcccttcggacggactggttgctgaaattaatttacataaattaatgaaacatccccaacgccaccttacccatagggcgtctcacgctatacggtctattttaatgcctaagaatttacgatgagcctataaataccttagttgtgaacgaaacgcagcacacgacaatcgtacaacctcacttttaatgttatatacgggcgcggcttggtaaatgccgtagctctagtaacataatgcatcctcaccataccagcaaagctaaaaatcttcaaatattcgtataaaactaaccagtttaacgtgtatgaggcggtctttttaccagtttgggagcatattgcacgtactatcttctttttagcagacctgggatctgagaacttcccctgggtagtcttacgattatagttagcctaatagattatttgttcgttaggaagaattcatatatactaggttatccttcaggttgaaaattaaggacgttacagatttttcacaattataccgactaccataagtgggagcgcgaatagcatttgagtatttggatcaagcatctgctgggttacacgtattaattagacccttgccgagatctagggaaacaaaatccagacccgcagtacgtgggtggtatgacgcttcttaggataggagcgcaagtccatagacctttatattactacgtttacctgatctaaataatctgatagaaaattaaccaggagtcccattaaggtattcaaccacggaacagagtataatctggttgataaagtcgttttgatctgttaaagatttgttaaactaaacgagacttctttgggtaacatcatacaagtctgataaaggatgatgcagggactagtctaaaatgagggagtctttgggtatccaccaaataatttcaggagttaagagcacttccaacgatgcagtcctttggccttctcgtgcgacaaggcaagaaaagtttataactctacagcttgtgtaactcgaaagctgacctactatataatgttattggaaatcaaactcagggttatcttcaaacagtttgttattggctagacagctattacctttaattggtccttaatcttgcctatggacatgctccacacattaaacatacttaatggcatgcaattatagattgtcccgttcattcactatagcttcataatggttggggtagtacacgcaaagtctacttatatgggcaacgcgccggcccgtctttcctgttaagttacgggaggtcgctaattactattttactgggaatgcgcaatcaaatcttgattgagaccaacgccaggcccgaactattcttattgttccagagtctttacttgaatgcatagtatcgggatggggtgatgccggccaccggatcaccatggatatacgtcagttggcccacgtgttaattaatgtcatattgttatgggctaatacattactgtattgtttaaatacaattcgtcatgcattatcagtactgtgtaatttatataagcgttcatcattgaacgtgtattttgttggtgcgtactgagttagatattggagaaattccctaaccaaggaacaatgactggacttgttagcgatgtaagagtaatgcaaaagttaatgagactgatattggaaacagtattgtttaggctagtctagaaataaactgctcataaagaatcttgcagttaatat

SEQ ID NO: 110 - DNA, 2000 bp, Artificial Sequence
  Synthetic
ttcactattaagtacacctagtcagacgtgaaagttagttcttttcacgtctcatatagtgctattttcgaccacgtcttgcaatcgtgatagacagagctgtcattaacaagatcaagttataaaattgtacgggttgtacctgcttatagttatatgttgaaattgcaaggccgcgttgtgaccggtttgacggaatctgaagggattagaggagtttatatttaatttctttcatgtagagatagaacccaataacctctcgctacatagaactaacgttttcgcagtgatttaccttgtgaagtgcacagtacacttcactgccttttactcgcatattgatacagtagccaaaagtatcattattagtgcataaccttcacctattccaacggttttacgcattctgcgtacgttcgattgaaatagaacaaatataactataattggtacccatgatgtaacattttacctcagtaatatgtcgaagataggctaagtccccagctagcgtaactagctaagccttgatgcgtattccttaatcttgtttaacgtctctgcttacgctagtttttagtagagcataagatagcaatttcaggatggaacgagttatagaacagaccactcctacagtgagtagggtcacatgtattgtccgacactgtttattcaattccaatcttttaagtgcgaatataataagaagcaccctttcaaacaattgttataatacgttttcatgacaccaacgatgtcgactatgatgtgcttctcttttggttagacatctttgcatttcgacgactccttttcattgagcaggttttagttagctaagtgtttcctacattgtagcgcattagtctaatagagagtgagcattagtcacaatatagtccaatggatctgagaagccttatgaggcgtgcttagggaacaattgcagtttaggcagaaagagttaccctttaagggtggtattcttatctcatatctatcttattggtgcaaagtttgtctttgaacgacagagtaactccattcgcagccttgctaaaagtggagagacgcaaaagtggaggcacaggtcgtttcttttagtcgtatatccagtttatgagcttcacatttaagatcaaatcccttctcgaaataaaaaggattcccactttaaataggcgattgattgtgcgcactatttattcgtaatctatacgtaaagaaactgaacgccacagcctaatacatgctagtatttcatacatgtgagccgaagacacgcacttcctttttgatgcgagaatttagggcgaccaagtctggtaacattctgtcctagttgccgagtaacatagatataagccttagcagggcgcggctataccttggtagtaagacgggtgtttgagtaatattagtagcttaattaacagcggtcaatcgccaaacggaattgtaactggaatgtcgtataatcccatttatatctcagcacataaatcaaaatggctgtgagatttaaagaggttagtaattgttcagaaatccgaaatcctcataaccaaataaaattcgcatatgcatacttgatcggcggagcgatgaaagaattacacttttagtatccaattataaacatcatttgcggcctacttttcccagtaaatcaatacgtggagaactggctcgtactctgctctacacttattgaatgagttagccaatgtagagctggatactaagctctagaagttactccagaacaattaccacgttaataacttctattattcagagtcgtaacagccctcaagtcctctcttgttcgcctgtcagcaatctcctacggacctaccctgccaggtagttgctgtctaagccactattagagttgctagatttgttaattataatgcttcgccatagtcatccacggtcagggcggtacctcgcagcttgtgtaagggatccctcgagtaactcttgatgat

SEQ ID NO: 111 - DNA, 2000 bp, Artificial Sequence
  Synthetic
cgtagtattttgtgagctagatggagtactccgattcaaggtattatgaacgatagataccgtggctatatcataggattgctacactgtaggttccagaccttagcgaagcggataccttccgttcggttatctgttaaaaactttacatcttcatgataaagtgtgcctacctttgtatcactgatgtacttccctacaatagatactctttaagacctgagtacgccgaaagaatctgttcgatctagcaacgacaaaacagttatcagcatatccgtatattgtggtgtagcgtcttcgtgtactaatttagatttctgcatctgtctagttacgtgtagggcctatgacggtcccttgcttttcccgggaaatatcaattgcagttgtgaaaattgtttataggaaaacacaaatctaaataaattactccaaggatcttctcccagatgactattcttagataatgagaaagggagactcgattaagtaatattgtcgagcaccacaatctgcctatattctaacttagtaataattaattaattatgagtcaaccaaagggtcgtttagctgattcatatacatactatatttgatcaccacctacgagcagttggcataatttccttgttgactagttttgacccacgtgattcccctaaattttttgtgctctatgaccgacaaccacagtgtaatgtctcaggtaaaaatgagtacatactacttttccagattgcataagttatagacttcggtattttccaaatattattgcattgtactacaaaactaacgggtatgagtagacacaaacgatcacgggtttcacttatgaataacgttgtaacgataagtgcgcctcgcctgcaccgcatcactaacgcctttttcgaggtaataccacgttccgaagaatctatttagttcctcgaataaaacattattgataagtagtgaatcaccagcctcccaaaaataccagaagagagaaacaggtctttcaattgctggtactatttgatatcctttacacgttttctattctccagtgtaagtctcgttatgcaagtttgtcaatatcagaacaatatgatatacaacacctcgcaagctgctagcagttagatgcgatccgatgatgatcgataaaaacttatgtactggacctgctggtttagcctttaagaataagttgattcttgacatacagctcgggcgataggattgaagagtaaaagcgatgtaaaccaggtctgtgttcgatgcagagcaagttcctgcatcggatttttcggatatgcagcttagatggttactcaaatccaattccgggctgttgtctgtacaatttgggaggttgacattgccacctgggcaaatgttgtccgagaattcgcccgatgagagaagggacttggtggagtcacaagaataggcgatttcgccccaaatttaatatccaaaagaaggcgttctactaaccgtaacgttagacatattcgtacagtgaagttcgcactatgtgtgcattactcaagtatctgttgtataggataccttagtggttcagtattaaacacgattcttttatcttgtatgttgtaatagcgatcgttacttatcaacagagttaaaccatggtacaagtgcacaagtcattaagcatctagactgcactacatcgcttctatattcaccatatgacgttacaatctcccaaagtaagtatgtgacaacttctccggccagctacatccggtagaattgtgttaactaacagtgtaattatactccatcatacgatttaaccggttgaatgactaaaacttaagtagttctcgcatgggtctccgcctcactggtaatatgtgaccgctctattgaattcgagaccaggatcaattacatcctcaccgggtaaagagtagatcaggatttttaagtgagtaacctggcgatgaatacaaggttgtactgcagttttaccctga

SEQ ID NO: 112 - DNA, 2000 bp, Artificial Sequence
  Synthetic
gatttaaatggtaattaaaatcgaaggttttaaaaggtgagaatttttttataaaatgcaatctgttacgcccctaatattcggtttcatgatttgcttaatattgtatcaacacaagcatattgttaaacagtctctgtactttcttgatgaccaataatgaacagatgaagtcttcatatattgaacttcaattgaatgcgtgcatgccattattcgtcatcgagaattaggaagaaaacaattgcagccttctagcgccaattgcgattagtaagcttcgccctcacgtactaaattatattagactgatcggagacattaacaagctgcttattccgtcttgaagaccgtatttcttactgttacggtgtccttaggcgtcatatatcaactaatataaaccggtactttattcataatagccgatattcagtgattgtttgccataggctactttctttcccaaatccccggtatcgctatcctatgatttctgcgtcaggggttaattacggcgacaccagcctaacccaagatcagactaggataatatttcactggcaatactcatcgattaattcaactagtatctattttttcacactccgcaaaaaagggcaaaacaaagtcgtcaagccgggaataagggttattcttgcagtcttcgtaataaaatttgaactcagttattgcgaatttactcgtataaagcttctattatcattctctgattactcaaaaacgctccatgagggtagtagcacataagtagaattgctcatagtggcttctttctctcaatccctttgatactcatttttatattacttacatgtaacgattgttgaaggccagcaaaccatataagtggacagaacagggaacaagagaaaataatacagaaagtagtaactagtcaagaaagtctagatgaatctataagttgtacctatcgaactatgatcgtagcattttcagtctacttgagggagaggctgtaaggaattttagcggccagatatatatcgctggaaccaagttatcgcatggaaacttgatcacgtacagaatgtgatgtacgcgcaaattagatctgaaatccctctgtcctcattttttaattaatacaattaatatcaaaggccttcttttctgaatgttattagacggaacacggaactgcgattcatcatcctaactacacaacacgaactgaccagatttgcgtgtaatcgtcacgtgccgttgcttactctagtaaaccccggcgcaagggcgaattgtgaaaaaatgagtcaattcgctacagtggcaaaaaacgagctcctggacgacacaacctcgtatagcaaggcgtagctcaatgcgccagatattcaggtattgtagcccatgacaacaagaaataaagctatagtaggcatcattatcgtttcgtccggcagcttttttctgacttccacctcattgcgtcttatgtcattactgcgtagggtcacctatatgagtcttcatccctgggacactgaagggagtacgccagtatttcatctatgaataaacctcgattactcctttatgagaacaatacttacactcgacggggtcttgtggtagtgatcttaagattatctaccatttgttcacccttgaaaaaagagacttacctctcgacttttttctatactgggccccgaccgctgacatgcagaatattgaggagatgcagattgatatttacaaaaattaaagcagatactcaacgcatattctatgaaaatcagggacacccagggtggtgctttaggatgatttacatgaaactttaaaaggaccgggataaactggccgccggtctttcactgccacagggatcttattcattcggatatattattgccactcaagataaattctgttagtaagtgttaaagtgtatcattattgcccattcttcagactcgagaacttcgaaggcaaatgctggacgtgtgtac

SEQ ID NO: 113 - DNA, 2000 bp, Artificial Sequence
  Synthetic
agatccacggccctgaaatcgccatcgctgttcttctttgatgaataatgcaagggctgagttcatcagtgtattcgaatgctactatatttcagtattgtgagtatcacagctgtaatcttcggaaatacaaggatgtttgtcgacctcgctaacactagattattttggcccgttactatttatatttttatgacttcaaaatgcgcttcaagattgtaactctggttgatataggatgcagggaccggctcagggccgctctgcactacattaatacctcagggatctctatttcgttagagcacacgacttagtgactagaatagctttaaatgtaaaacttcatcatatattcctcctggctaagccttaatttcattcttggggctgttgccaagactgctcaagagttagtttttctttctccttgtagtacccgttctcctaagtgcaaataatctatacacacttcatattgggtataccattcttggtttattgtcacctgttatgtattttgcatcaaaataatcatcgatgtatacgttaacccaggagacaatcgaccggctaattccgggaacgtagatgtatgtaaagtaacatgtatttcaatttcttctgaagtatgagatttcagttgcacaaaaggtactcagcatgtcttatcatccatagggccgcaattatagaggatcttgagtggagggtccatacgaggccttaggaagccggcttatctcagcgaaggttatcgagatgctaaatttacggataaagatccgttactcttctttagaactaccgttccaactcgaacatagaatcggctccgaattcttgggtaccttgcagaactgaaaaatagatatctcggtatctaaaggcagaaatagttttcgctctggattggtttctaaagtgaatctcaagttctaggtaagcattcaagtccattggggaccattaggggttaatacgcactgacgtcggtctttcgattgataaatacttaacctcgttagcagtgagggtcaacaatcattaatctccagctatagagcgggttagccagattttatatcggcgtcattccttttatctttgaaatttaggccaaaaagaagggaactggttctattcgcgaattgaaccgcatttatggtaatagatctgaccacgtgctactgctcacttacaatagctagttttcggctcaaactttgtataaggctcactaggcatataacgagttaaaacttttcacatgatacgtgactagcttcgcccgacatactatatataaggtctaccgttgcgggaaaagatgaagatgatattatcaagtctttgactaataaattaacttatgcttacaaatttccaaaatagatattccagtcgtctatccttctattacagagaaaggcagacttaatccgttcattatataatttatttagatgttagtctttctggtgggtcgattgttagtctttacatagaactcctttaatgttcataagtttccatcagtagaaagtgagcttatgggttattcacctttgatattaaaagatttactactgctataatctacctagctcagctgagaggcaagaggatcacatgttattgttataatgctttgattggtaaactatagtgtcaaggcaattcgagtgtcgccaagttacgtcgattagatcgatcattaaaatctaataatgtttagagtttgttagagtaatggtgttgatcggcacataagagtcagaacgcgggagtattgatattttgccgaattgcaaatttatcaacatcggttctacgtatcgttgatgtcctaaggccttagttacgtagcttacatttaatgcgcatagggttgaagcgtgtgttaatcgctctttcaaataagtgttaggaaatatacgaagtaacgaatatcagcctaattccagcgactaaaatgaaacaagagcatccggtggtag

SEQ ID NO: 114 - DNA, 2000 bp, Artificial Sequence
  Synthetic
ttgatagtgtgattaattagctggtcattatcggtatcgttgacaacagtaggatgatggcgattgtctgcagatttcgtccattaatataagtaatacttgttatgatgtccaacttagatatattggagttttattgctctatttcctgtacccttgtgacgagtaactgctccgtgatataggcaagttaagtgtgtcgcaatatggcagtaggctgaataccacacatactgtctttctaaataacactaggcgactacctttaacttcatctaaggacgttatttcacactaagcactccgtcccgagaacagggtctattgaggctactgattgcgtaaagtagttggacacgcatgggttctagatcctcatctctggtttctcaacatattgagttatactttctgttagttgttaagccgggcgatcaaagcatttctacttcagaaatggaggactgtagttatatactacattctgaagcggtaccattaatgctttccgcattgatgaatatctatatttacagtttggtgaacacaattaggagagtcggactgcgcaaacagaatatttagttacttatagttaatatacacctatacacggtagaaggtcagttcatatagacttctgggtgtgtacttcatcagaagtctcctgtctgtttagccaatcgccaccttctcagtcccgtgggagtaccactcgaatagatcgttgttttcgttgttgataaacggaccccgtcttattttcgttaccatttaatacgatatcatataattgaaatattaggaaacggcatttcaaatacgaacgatttgaacttcacctaccttttgacatttatattacaattttatagggcaaaacgtaatgcacctaaatttactgcacttcagatctaccaattgatttgtcacaccagctatttaacgaacaatatgactaaatattagctggtatgcaatctgaaaagtcaacatggtatttctgcttacaccggtagggttaatggaagttctgcgcccattcgaattttagaactgaacaataattcatgaaaatttacgttagcagtacctttttgtcttactagttgttgcagaaatttaaacattacttggtagcctgctgtgtatataaaagagcgatctccgataagttgttaatctgttgctacctaagcgcttactgtgtgccttggctcgcgtatatgcccaggtcaacatttatttgtcgctcgactcgaaataatctatatcataagatgggaacgagtatgctccatgagggagccggactaggcattcaattttgtttgagtctttagtaaccatacctattcatgcgtagttaacttcgtagtaaagcagcgtttatacataaacaccaaaaaatgtcctaggggcataccaagaatctaagaaacagcgcagtagttcgttcggtttggcaaccatacgaaagtatcattgcacacgacgcatacagcatcctaggagtttactatgtcttcgtttttttgtaggccccacacacattaaattcgatttattacactcagagtacctgtccgccaattcacgtgagtaccttcgcgcagcagataatacattgctatgcgttcagaccattgtaagaaaacagatcatgactctagaaaaagtggccttagatcaataaatgttaaatccggttctctctaacctcgccgtacacagttaaaatcaacgcgcatacataaacattgatcttatgggggctcacatagtgagacaatagtagtacccagtgttatacctaatctaatatataggctaaaaggtagattaattgtctgatcatagatctcaaccgatcatggatagctgggaatacgttataaaggtaggtctacgacccgcgaaatctcgaggaaccacaacagaaaccattgtctgtacgagcgacagcgtatgtactccgtggctggtctacctcggtaatg

SEQ ID NO: 115 - DNA, 2000 bp, Artificial Sequence
  Synthetic
gggtagttttttctccaaggatccccttaactagggtgaagattgggattaaacctaagataaagatataacggtcactggcgacaagcttacaaatttgcgctttacaacagaccaaggcgaaagtaatcttggccctactaaaccaagggaaatcagtagtagtgttctccaaataggcaaggctaatatctatactgtccctgcatgatgtgttaagccataggcgtgtaatgttattccttttcctaaccagcttttaatgtatccttgtgtaggaagaactgcgaagttatgttactccgaagccaaccaacatgtgtcctcttggcaccatgattcgaaggtgatattataagttattcgaccgtgaagattacatattactggatggtgtataaatagaccatacgttcattgaagcgtgactgaagccgacaacggcttacgtaatgattcaaaatcggtaataaggataacggttatatatagtagaattcgagatggaaaaaccaacttgctaatgacaatattaagggtatatcacactgtggtttgtaaagtagtcacctattcgtgatgccgtgtacttcaacttatagtaaaaagtattgttttctaaccagcggtaacctgttgcaaaaaaccacgtttaaccgattgatagcttgtggtaaagtggcatagagtatacttcctccatctgtagtacttaataggtgttccagttgcagtataaacctttcttcgagtatcatcactaagaccattagacataggatatatacaataagagctggaacttgaatcttctaatgacagactttactaattatagttcaagcgcagtttaactataaatacaattgtcaattcatcatatggtaggcaagattcctttagcctggcgtacagtggcccggaggccttgaccaaaacatggttctgttatatcacgagatggattgactatgctcgtgaatctggagaggcactaacttggtaacgcccgtactctaccgcagcgggacaggtgatagactgtctatgtaaatcgtcatcaatctatatttcaatacaactataaatccagacaagtatccttgagataatagttaatctatcctaactaataagaagaaaagagacgatacggtagtagattaagctttcgcggaaacaagaggaatctacagaaaacaccctaaataagctattccatgccgcctttgctatgaacgaagtacggaagcatgatgcttatcaacgtcaggaacctagctcaaatcaaggtcttaccagtgacgataacatgggtgcggatggttatttgtggagaggcgtaatacaatgtacttgttttcaggatatcaatttaatttcacttagaatacgagacggccgacaactttaacgaatacatttgcatcccacattaatacctgagtgccgctcatatcgtcctagcacaatttttaacagaagttttggtggtgagtagaacaacaacatgtagtcatcttaagcgtatgaaatctggctctcaaattcatgtttaatagtgtttaatcttttatgtataaatcgtttttatggtttagacgaagcactcaaaaatatagactgatgcctatgacctgtgctatctttattttccagggcaaagatgatctttccgagtccatatcttgaatgacttcccgcctgaaccaatacctggtcggaaggaggactcattaataaacatgcataaatggcagatctgaactggacggctgacttatctcacaatgtgttctaaagtccacaccgtttctgtaccaatgaaaggacgaattatacatgcattggtttggttaaaaccaatacttggtaacgatctggaccgggcggttagaatgatgaattaatgcgccgtatgtggaatgaagtcctgttaaaatgcaaaaggtggctcttcgagagttgttgggttgaatgagagaaacgccaccttcaca

SEQ ID NO: 116 - DNA, 2000 bp, Artificial Sequence
  Synthetic
tagtatctagtttcaggtgtgcacagaatagttatcctcctttgtctgtggctatttggagaacgtattagaggaagcatatggcaaaatggcctgtacacgatagatggtatcatgtttggaggacgctaggcatttcgccctaaacaccgcaacgatacctaaagagctcgtcaatgggcttgccgattaaatacgcaagttttagtcagtccagaccacatttaccggtaattatgcacagacaagatattatgctggtttatagcccatatttgtctccccctaaagtgagctctgatatttggttaggtcgagtagtacagtttgctatctatggatacgatgtaattgtgcttgagatacgtgcatcacgaacattgctaagcggattcgcaatgttcgtgatgcatggagtagtctaagcaatccaacaagcgcctgaatataattttgtcacaagtaaaccttcatattgtctaacatacagagctgttttaccccctcatgatctaaatctttcgcttcttcccaaactgcacgccctattcgcctgttagcgcattcaaccctaatacagctgttgtggggatactctgattgaaacaaagttctctatggaagcttcatcattaggccatacgaaatagaatcccctgttgtccaggtgcttctcgactgcgttgcggttcttattttggctttgctaataggaacttctctcttcgagctcggtcgaacgccagttcgtcaactataccgccttctttttgcgcaaggtcatcgaaactgaggtccatcctgggacaagagatcagttaagcctacacttgtgtgagactccgcagaaaatcgggaccaaagcgttagggcttcccaattatgaggatctatggtgtcattgaaattgataatccttatagggccatttttatccctgacctgaattctatttggtgaataaagtattggtcgcctttcgagggatactactatgttatggacctaatggatgaccatctggaacattagcaacagcaactctaatcttattttatcatcttcagtgtaatatatcgtacattttaggctttcctttatgttaaattgttattatgaaagaggtgtattataagctagttaagcgcgttaaaacacaagtggtctgctgtcattcatataccaaagaaggtcttgatggacaatgtcttcacaagaccatgcatagattctaaatcgatatgacacctaacaaatgcgggctaatattcgatttctgactcccacactgtgagcacgtttattgcggagacttttaagcgagatactcttactccccattgccatatatgtaaaatggacttccaattctgcatatttcagtacatccggactgcgttataagcattgtcgtggatgcatcaccatcccatagttccacttcttttttttagttcagatccaaactacactatagggtgacttattgtcgatcaaaattattatatgtaagtaatagatcatacatcaacaccgaggtctttgtccaatagaaatagtatgtcctggagttttatcaaatacctgccatgtgcaagttcacagaataggacgcttctacagaattcataaaatcccacatccttagcgtaagttgtcagatgaattaattatatttttgatacggccccagttattctcgaagtccactcttaaaaaaagttattgtacgaacttgcataaatcgataacctgttaccaacatgccccggcataaatcaacaacgtggttcggatacgacaatatcaatcaatccgaaattcaaaatagaatattcaacttgacttaatcgcagttcattcgtgaatagacacatattagctctcgcgcgctttcttatcttcacagcttcttctcgatacctgaataagtacgggaccatttatgttcataagcattcagtgaaactgcagtctaaatactattggcatatacttat

SEQ ID NO: 117 - DNA, 2000 bp, Artificial Sequence
  Synthetic
gatatgccatctatcgaggcctgttagcttaggacattacatgacagtgagacctagatatatagttgcatgagtagatgtaaccgaaggtactcagggacagaactgacggattgacgtttttcagtatcgtaaaagtttgagatccaacaatgaaagcttgatgcgccagatgatggaaatgcgcaaactgtcgtgtgataacacgggaattggtgctaagctggaatggtctaattcaagttccaatccatatccatctatgtgcgaggaatttgtaacggtaattatattgccttacaattattatcaaccaacacacttgaacgatgtaattgggggtatataccaataatagtactgccaactactgttttttgcaagaattaatcgtagtccgaattaaaagaaaagacggtgtacgcaacccaagtaattaaacgaataatcatacggtcgatatgctcattcgataaaacgcgagatctttaagttctctcaccggggtaatgcataattgccttaattggaaattgctttaggtgagagtcagtaaaccattggtgagatgtggttatactgcacctcacgcaaattaatattctaactttaacctgaattatgggttcccctcatcgggaagtatatctagtgccaacctatcacagttgcgcacatatgtttagaaatggttagtcggtcaggggaactcacgtaagcggtagtagtagaatttaatttatggtctcctaaagcatcgacatagtacactgcgaccattctaacacatactaaactttgaacttactgatatcttttatgtttgacttccttgctacgcaagtccaggcccagacagctgagttgtccttacacgagctatttgctgatcatatggtttaatcggcacgcgaattgcaagtttgatttaaggtgagcgcatacttgaatacagccagggagctccctactcagcgatcgtcttcagagatttcacgaaaatataagcattcccatcagaaattctaattaaaccttaccggaggtggggattactcgcagagttaaataatgagcccacattatgcgtttgcttctggagattatgggtggtttttcccgtaccgcctaatatagtatgcttcgactcagcaacttcactctaaaccctagagagcctctgtatgtacgcgcgtggatgaaatcaagaatggttggagtcaatgactggggcacaagtgtaatctggttcgattaatacatggcactaggtgctacgaggacgagtgaatgcaatatatgagtccttgctaataagcatcgaagatactctccggtactccttcatattcgactaatcggtgcactcaactttaggggggctccttattataaaatacatatagggtttgtttaaatgatttgttctattaatacgggcaaaattaatgcaatgttcacctaggcacgttggtactcgccgccaaacattggcattaatggggatacttagaaacaacataacatgaaaaatatctaggaacgccaacatatacgccgtgaccgtctgtcttaatagactctttttgtttaaagggtactgagtgattaactaatgctttccaatcctttccgttagaaggctattactacaagtgtttcccacgtgccgttaaaaatagaattatctttgtgggtttacgagcgcgtactgaaaacaggtttcttggatgggataatattatagatagcaataaagtaaactggaaaacagtattggatagcatgtgatggaccttgacccccttgtggcataagataatctcagcgtttcgttacacttacattcactgttaatgtctataggcaagttactatttggagtatttcaaagtgaacggaagaaatagaagtgctaacaaactccgtcatagtaggatcatatctccagagcgacctcatacatgctaaaaacctagtagacttcgtactatggattt

SEQ ID NO: 118 - DNA, 2000 bp, Artificial Sequence
  Synthetic
aagacactttaccacataagtaaaccgttgacattatcgtggcggagagatactgcttgtactgggacactcagtattttgtggaatattgtacctagcgcctcgttccgtgaaagtgtggcatggattttcataattttatgctgtcctcattgcctacaattaatccagtaagcactagagaaatatctgctcctatgctgagattagccttatgaggtctttatatctttctgtaaaggccattgttcttttgatcctggagtctctgaattttgatttgtccctcaaagccttatgtgtacccggtcccggagcatgaagacgtatatcttgaagtaatccgaaagtatttaggtgtcgttgtccagtagtaatcccggttatgggttataattaagtgttaacatccgagcttggtctgtataatagtgtgtttgaatagtaaatatcaggactctacagggacctattctacttcgggttgtgtatcttccttggaataacttttgctacgcaaaaaagctataacaaggtctggagacggatgtgatttagtagggcaaatagatttaggtcttcgatagtacagaatactatgctacaaccaatctcttcatggctttatcaatacaatgttcttccttaactcagacgggagcaattatagttagctgaaggttgcctcacaatatgtgtcagagctagcgaaaagctcctaccaatatacatcagataaggagttcatacatctgtggccgatcaagcaagcaaggccgtccggttcacgacctgggtagtctgagtttggaggagaagccatcgcctctcgcattctactagagaaagatttcacacttactgacagagctacactggtacgacgaatctacaaaactaagcaaagtcctagggtgagcaatgcatggtaactagtacgattgatcagtgcgtggtatactatccggatagtccagacgtcaagacctaatcatcgtacgtaattaaataataatgcattcaactcttcggatacgatatatacttatatgcattaactatactttctcatgcattgtatctaacaaaatctgtacggcagaattaattactaaagtcttaatgattcgaatattaatatcaattttattacgaaacaaccaaactgacaacgtagagaggcaactacccagagtcgccaagaatactgtttacgaattgtagaaaagatgtaagaatgttcggatgtcggattacttaattgcgaacgtttgtcaagtcgttgcaggataccctcatctcctcttcctagtgaattatctgaaagtactattatacaatctaaatcggatacattcgtttgtaacaccacatggttggctcagctgaccatttacgcgcgatattctgtgctatccgaaggcgtaaaaggaattcaagtcagtctcctcttcgttatgtagaaagggaggactcctccgccgtatattcagctggctttaactaggaacatagttgcagttcaaacagtagaaaatcctggaagacatttcttgatagtctatctcagaaaaaggggggtgacgttcatgtttactaagacttgaaatgtggctccgtatctgcacaaccaggtttgggcggatgccggccgccatgtaacactgaacctcgcaagaaatgcacaattgaacaaatgaatactcacatcttatcgcttaatgttaaattcaaggcgagactggctcgaattattggagcctatgaagatgtatattaatgccaaggcaccgcacatagtaaagactatactaaccaagtgtgatattcaatcgatcgttgtggggaatcaggtacagttagtggcgaacagctttgacatccgtttaactttggcagcaccacaaaccctttgcgtacgtttttgtgttataaccaagttatgttgcaacctactttgacctcttatttctttgccgcaagactgaatgtcgtattat

SEQ ID NO: 119 - DNA, 2000 bp, Artificial Sequence
  Synthetic
gagcaacctacggatatactatcgattctggacatggtaagtgtgttgcgtggttaataaaaagatttcgtggtcgggggtagatatacctgtaaggtttccaacagaccgctttgtagaaagagacttagtccctttgcaaaatgaggggaccgactaagaaagcgttgaattcaggtaatactttttgacgttaccatagttgttgcagtcccggagttaaacagagacacatcgtggcggagtccgtagtatcgcatgcgtggatttattgttgtaatcagatgttcaatatggcgtcaatatacaaataaacaggtcagatggagttagccttacttaaaaaacgaaaacaatgtatgccctaagcaaaaaaactagataaggacgatcaccacagttttaagagatctatatgcccctttgacatccttattctgacaatgggcagatccaactacaagatgtcgtaccgctaacacttgactaactaacgtcaagtaaaaagttcgttagtcatattatcaagtatggacttattcatcgacaggttgtaattagccctcccctagattagctgggctgaacccctattcctacgctcccttgtcacatgtattctctacctcaataggccggaaactcgcaagcccaagtatagcgtacggattaaattcgcgcaatcgctcttgaccatgttaaatgcttgcgcgtaacatcgaaaaggaggcaagacatttcagaagtaacatatcagttgacggcttacggtgctgaggtttaaaatccgactgattgctatcctatcgctgaggaatgactaaccttgcaaatccaagtctagaactgtcctagttctgtaccatgcccagcgttcggatgtcagtacgtgtatgcagcatttaggaggtgatgtctcccagtcggtcaataagctttgcttacctcacggataactaagttcatctccagtgtacgaagattctctagcactaactattcattgtaactaattggtatccgactttaagccatagtgtggcatgacgtaagttatgtcagttctttggaactttttgcgcagctgtgttgacgaaacacaggttgcaggttggtctaggtaagggatgcactcactgcgatgtgatcctttaatggccatttaaatctatctcgagtatagcgtgtatacttactatgaagcaaattagtatacatataacaatgaatatacacatagtgggaggttgccattcatccatgtaggcatgtaatatggcacctcctctttggatacagaggcccatgcctccgaatcacatatttacttaaacagttaacggaattcaggtatcccgtttcattattcgaaacgtctctggggttaccttacttacgttatctgcatgagaatagagtccatcggcgtttctaacaatcaatcatgcttgcaattcagcgagtgtagaggaattgtaagaacgccggatgctccctttaccttatccgcacaggcccctacgattgaactattgaaagttttattacaaatctcatatatgggggagcagttaaagttctgcataagaaggacctaggataatgccataaaaggttgatatggaaatactattggaataagaaagtatatggtgtctataatggatatatcagtaaacgaaggcatttcttacactttgatttcattaactgtaatctctatttgtgttggcgaatccggtaaacagaggtttataactggtttaccttagtcgagtgtcttagatatacatgtcgattcagatcaatcctactcatcccaaacgcacatgtcacgatacgtactttatacagtaagaggcacaatgtgggtgccctctctcgtccgacttattgcggacggagaaatagttagtacggactgtcacaagtctgtaaccactaaagatcgggcagctcagacattattgaaggtaggccaaagtatcattaatgctttg

SEQ ID NO: 120 - DNA, 2000 bp, Artificial Sequence
  Synthetic
attaataaatgtctaacggtctagaaatgcacctaatttgctactgctgaactcctgattactcctcctcgtttatacttgttcattaagaattttttccgtctagattaagtacacggtaatacacacgattaaatacaccgccacagatcttcgctatcaatattacattttgttcactcattacgataagcgtggcttggctgagttctagacttatcgtgttaacgtcaatgaaaacttatggatttgaagctacgatgctaatctaactttaccttaagcaagaaagaccttcgttaataggacccttaaagcctgtgatgtcggttaaacggttctagtttgatagtgacgttagggactcggtatacatcttagccgaactgtctaaattactttagagaaacttttccctgggggaggcacgttccgtttatggacctcatttgagactcaatatgtacaactaatagtgtgattagatcctgattcccatacgtatcggctcgcccttaatcaatacagatccgtgctatgtccatactgcgattccaaaggttgtctaacaagacaaacttgagagaggcttcacaaagcaacccagcacccttgtcctcttttttaggggtacgctgacatctggatgcattaagaaatacgtatctagaaggatcgcgataagtcgcacaagtttaccaccttatattctgcaggctgctattggaggtaatacgtgctcgcacacgcccaagtgaggcattcttacaagacttaccttacagcctattaataacgtcgaattttgcgcagcaaccaattccagggcaaactataagccttattgaggttaatagggcgcaatatatttacgatagaaggtaaatctataatactgtcacttgtcaatgatgatggtctaactaattgattcccatgcaagtggcgaaccaggcttactttagtttaatagcgatcaagtatactaagcacacactgaatgtatcacataagatacgtaaaataaatcaactcattaaatcaaagacagattcacaaatgtttcgtgttttaacagatctgaatataaactctgctgatgtgatcgtaggacgtaagaaggtatagttgaagaatagcgtgaatatctgatctctgttagcaaatacatcacgattatcaccaggtttaccacaacaataagattgtgactgacactactttctatatgaatgtattctcatgaggatgcgtaagacgtataggatcatactgaattataactccatattagggtctatatcacatacatctccaagttaaaaagtctattggcgattccacacaactcgcgctagtagtacattttaccggtaccggtacagtctaagttattgatctaggttcaacttctaaaatactgaagtctcaggtatatagaatttatactactcgcgggacgtaaagcccctctgtggttagcgtcgcagcgtcgagtaaattccttatagagcctaaaccttgataatttcgacgtaccgttataacgcaattaatagacttctcattttcctgccgagtcgggtctggtatagtctaggacgggggtagatatgatcgtcgtcttctctaatctaatttaatctataaccacagcgtacaagtaaggtatgtaagatacagagataaattagagatttgtgttactccgcatgttgaactaaacccaaaggttcacgccgtatgcctttcaagttcctccgctcaaaaggctccgggtgtcccctacccgatatggcggaaatcgttaattctcataacgaccaaccttaccttggacacacctaagcactaagtcggtaaatggagtacacaatgtgggagttgtgtttaacataatgaggctcgttcagactatgttcgaggcgtataacgatttgtgacagattcctcatcaactcgggtcagatttatagcaatggtaaattccctatatccta

SEQ ID NO: 121 - DNA, 2000 bp, Artificial Sequence
  Synthetic
tatggtgtggcacatatgaataaaacaaggagaagcagccgacaatacttagaacgtgtcagaacaatcaagatgtctgaaacgttcaacaatcgagttattccgggctaatttattcccatccttatatacagagccgcacaataccaagtaacgtgctttgggccacgaactcactctagtcttccggaccctccggtactactcggtatggtggatattcatgagaatggttttagtcttaaaaaaatgtgaacaagaaaacatttacgtccaagaaagcggtattttgtttgggtctaggaaacaatcagtcgtggacctgggcgagatcggctgttttcgaccgattttatgctaagcagaaggaagtgaccgaggttgtgtttagatccagtaaaagtcgtcatacccgaggagatttctgtggtgcctagtgactagcgatcccgtgcagcagttcaaatgcgctggatagttcgctcctgcaccactagttcacaccagaagtatgtcttttaagagactgtctaagaaatatagtctctaaacgtgactatcgttcactccctgtacaaatctaggactaacgggtatagattaaacgtattagaatttcggagcattagaattttgttgttctaagttaggatgatttcaagtgtccatgtaaattgaggtcaatataggacgatctacatccgagataggccaagtacgattctgtgttacattttgcgttcgcacaagctaggacgagggtatgagcattttgtgctaaccgaatgagatgcagcttattgtatccttacccgcaacatagggcatgaaggcgtggttcgagaatcgcgcgagataaatacatgtttcgatttatgtcaaccactgcaatggtttataaatgttattcaagcatcgattcaataacctctggatgtagtaatatctgcgggtgtgtaagtgcgatatcctaagtcgggagatttaacaataccttgggatgctccggacaattttcgacgtacgcaattatgaacatgcattgattgactaaacttaagaaacataatcagtgtatagtattgtaacaatggattctgagtgtctaatgttttctcgctccatgttataacacataattatacttataataccatcccatctttaagtacaaaaccttgttgcgctgctttatggagactattgagcccaacgggttgagtggttattactatttgaagtaaaagcagtatctactcagattcctagaggtaaatatgaacttgttttctatctggttatctatttttagttttatggatatggacgaagttaaaagttatagacctgacattcttctcccataggtatagaagtggagttaaacaagttcttagtgggggaaatgacgtacagactactatcttgatgatagcttttcgatcaaacaagagtttcaaccgctgtaaaggtttatatgcgatgtagtgtggtacgataacgtactttgccgatcattcactgattccattaggtacgacactctcagttacaaagcggtactaacctagcaaaaagtgaatatcgccctacaaactattactggagtgcggtggcagctttggcgaaaattggccgaactctttgctgtttatatggtaactattctcactatgctactgattggaaaaagatatttgccaactaatagtcgtaatgttagtattgatagggataataggcatttaaagttccctgaaacatacggtaaataagatctcttttaacaacaccaggggtggctcactggggtagcaaatacttaacgatccctttttcatcaagtgagttatctgctttggattcttacaactagatgttataaagaaagaagctgcgcagtttgcatgactaaaatttatatgaagtagtagttattagtactatctcttagtaggctagaatgtaaacctgcagacatcatggaatgcacatacccg

SEQ ID NO: 122 - DNA, 2000 bp, Artificial Sequence; identical to SEQ ID NO: 23
  Synthetic

SEQ ID NO: 123 - DNA, 2000 bp, Artificial Sequence
  Synthetic
atctcatagataactctatgaggagttaacgcctagaaattttggtctgcatggtacagttacatatcgtatgaattcgtctaacatttgaacggaccacaccatctgatccgcactcaatggacagtaggcattcggttacactttcgtctggaagaacagtccgaatatgaaaatatgcttagatgattccaagttaatttcgtctataaataagtagcttttgctctataaagataacctcctacagtcgtaacagagctcatatacgataagaagagtatacttttagtttttcgcacatttagccattcaatcgagaacatagacgcctcgagccgaattgcttagcacattttcctaataaatgtattcgaatatccaaaatgaacttgcatgactccgtagcacgcactagatttagtgtgcctaaagattaatatcccaaggttgggctagaactaaaaacgctgttgccaataggttagattgtaaactggcccttaacaagctgattatcaggtgctttggatacttagcacatacttaacacatcggcgtgaataagtgggaaaatgtgcacaaactcattagaaattctgtgattgggtctttacgttatgttaaagttggtattgcttataataacttattctcgcagcgtactcgagaacgtttgaattcgtgagagcccttaaatcaacgacccccggcgtttagaaacggcaatccatatacctgtcataaattatcttagaattattattataccctagccttagccattttgtttaccagaacacggatggatctagttacgattcatataaagtgagagaggctagtgttgtaagggagtgagagagcttgcatcttacgagctcttagctcctcttatcaaaatatcatttgggcccaacaacgcgtaagtcagatgatctattagcagtttggatatgttcaagaagtcctccagcgggtttgcgagattctctgtatcgttgacttgtgacatatgatttgtattccaagacggtcagttgcaatcttgcctgaactagttggattatcagccaccccaggctgttgcatctaattaagttttcctatctgtaaaacctttcacttagcaatggcttaatgctcttaccgatcagctggaagccggtagtactgtcacttggttttcttaacctatcaaaacggaaacaagccgtatttttgatggtagcacttcaaatggtgggcaaccgactaaagaacgtcactctttaaattctcataagttaaaatcggatgtcgagtcaatattttgtcgggccatgggaaagagagcagtatgctaccttcttaatctctaccttactttagacaagcatacgtcaacaactgtgactcttcaaggacgggtattccctgactcaatgctttggaagaacatttaactgggttccattatagtggtcggactctttatgcttatgtcgcaccaggtccatctatcgaattcctgtattctataaacaccggctgcactctaagaaagatcgagcttctgattccaaaagtctataaatgatcagttagcctagcgccgacacattgctccgttagaagcttgacgtttgttattatgagggatcacagattaccgtgtgtcgattggtggctcacttatctatgagccagtttcgttatggtcatacctttaattaagggaacatcgtgctaaaatttttagaatggggtactgtctagactgtctcgaggattcatgccgatgaagacctgaaatttgaatcggaacttttgtggcaccgccgtatcgcaaaatgagaaaaagatatcgttaaccccttataaaccgcaactaactaagtcaaaataagtcgacgtgacttaagatactgattaagaaatggtatcacggctcttttgcaataccattaccaaaattgccaatgaaactgttttggcctatcttaagccacgaataatat